(19) United States
(12) Patent Application Publication
(10) Pub. No.: US 2005/0221285 A1
(43) Pub. Date: Oct. 6, 2005

(54) Method for identifying or screening anti-viral agents

(75) Inventors: Craig James Morton, Glen Waverley, Victoria (AU); Michael William Parker, Victoria (AU); Jane Ryan, Victoria (AU)

Correspondence Address: ROTHWELL, FIGG, ERNST & MANBECK, P.C., 1425 K STREET, N.W., SUITE 800, WASHINGTON, DC, 20005, US

(73) Assignee: Biota Holdings Ltd., Melbourne, Victoria (AU)

(21) Appl. No.: 10/492,187

(22) PCT Filed: Nov. 8, 2002
(86) PCT No.: PCT/AU02/01522

§ 371 Date: Apr. 9, 2004

(30) Foreign Application Priority Data
Nov. 9, 2001 (AU) PR 8784

(51) Int. Cl. C12Q 1/70; G06F 19/00; G01N 33/48; G01N 33/50

(52) U.S. Cl. 435/5; 702/19

(57) ABSTRACT

Anti-viral agents which may be effective for treating, for example, respiratory infections by Respiratory Syncytial Virus (RSV). A three-dimensional structure model of the RSV-F protein has been generated and described which can be used to identify, screen, and/or develop anti-viral agents, including RSV neutralising antibodies. The structure model may also be used to develop RSV-binding antibodies useful for diagnostic assays.

### FIELD OF THE INVENTION

The invention relates to anti-viral agents which may be effective for treating, for example, respiratory infections by Respiratory Syncytial Virus (RSV). More particularly, the invention relates to the generation of a three-dimensional model of the RSV-F protein and the use of that model to identify, screen, and/or develop anti-viral agents.

### BACKGROUND TO THE INVENTION

RSV is the predominant cause of acute lower respiratory tract infection in children, with symptoms ranging from severe pneumonia and bronchiolitis to much milder infections similar to the common cold (reviewed in Cane, P. A., Rev. Med. Virol., 11:103-116, 2001). While primary infection does generate a long term immune response this is not fully protective and recurrent infection can occur throughout life with annual re-infection rates of 3-6% in adults (Monto, A. S. and Kim, S. K., Am. J. Epidemiol., 94:290-301, 1971). Subsequently the virus is also a major cause of morbidity and mortality in institutionalised elderly and immunocompromised adults.

RSV is a member of the Pneumovirus genus of the family Paramyxoviridae, which includes other human pathogens such as mumps, measles and the parainfluenza viruses. The RSV genome is a single strand of negative-sense RNA encoding ten viral proteins, three of which are present on the surface of the infected cells as well as on the virion itself. These three proteins are the G, F and SH proteins; G and F are responsible for binding of the virus to target cells and fusion of the viral membrane with the target cell membrane, respectively. The F protein is apparently necessary and sufficient for viral infection to occur as mutant RSV lacking G and SH protein are still able to infect cells in vitro, albeit at a reduced level (Techaarpornkul, S. et al., J. Virology, 75:6825-6834, 2001). F is also expressed on the surface of infected cells and syncytia formation is a result of fusion of neighbouring cells mediated by the F protein.

RSV-F is expressed as a single precursor of 574 amino acids with several sites of N-linked glycosylation. This precursor molecule F₀ oligomerises in the endoplasmic reticulum and is proteolytically processed at two sites in each monomer, resulting in a trimer of two disulphide-linked fragments: F₂ (the smaller N-terminal fragment) and F₁. The protein is anchored to the virion membrane through a hydrophobic peptide in the C-terminal region of F₁, and is believed to adopt a metastable prefusogenic conformation until triggered in the presence of a target membrane and/or receptor. Triggering the protein is thought to result in a conformational change that exposes a very hydrophobic stretch of residues at the extreme N-terminus of F₁, known as the fusion peptide. It is generally believed that the fusion peptide associates with the target cell membrane upon exposure. The present inventors hypothesise that the change from the prefusogenic structure to the final fusogenic conformation of the protein brings the fusion peptide into close proximity to the anchor sequence, promoting the fusion of the virus (and/or infected cell) membrane with the target cell membrane.

The structure of a fragment of the fusion protein of Newcastle Disease Virus (NDV) was recently reported at a resolution of 3.3 Å (Chen, L. et al., M.C. Structure, 9:255-266, 2001). This protein has some similarity in both sequence and mode of action to the RSV fusion protein. Only 355 of the 553 residues in NDV fusion protein were present in the final crystal form (residues 33-105 and 17144), such that important regions of the protein structure were absent. Another structure was subsequently reported for the core fragment of RSV-F itself (Zhao, X. et al., Proc. Nat Acad. Sci. USA, 97:14172-14177, 2000). This structure included a large section of the protein absent from the NDV-F structure. In the examples provided below, the present inventors describe the structure of the majority of the RSV-F protein produced by molecular modelling on the basis of a chimeric model devised by combining these two crystal structures. It has been found that the surface of the protein contains a region (“Site A”) vital for the activity of the protein and into which compounds known to inhibit viral fusion probably bind. The present inventors suggest that the region is important in the sequestration of parts of the RSV-F protein in its prefusogenic form and that it plays a key role in the process of cellular invasion by the virus.

The process of rationalised drug design requires no explanation or teaching for the person skilled in the art, but a brief description is given here of computational design for the lay reader. A person skilled in the art may use one of several methods to screen chemical entities or fragments for their ability to associate with a target molecule. For example, the screening process may begin by visual inspection of the target molecule, or a portion thereof, on a computer screen, generated from a machine-readable storage medium. Selected fragments or chemical entities may then be positioned in a variety of orientations, or docked, within identified or possible binding pockets (ie target sites). Docking may be accomplished using software such as Quanta (Accelrys, Inc., Burlington, Mass., USA. COPYRGT. 1994) and Sybyl, (Tripos Associates, St. Louis, Mo., USA) followed by energy minimization and molecular dynamics with standard molecular mechanics force fields, such as CHARMM (Accelrys, Inc., Burlington, Mass., USA. COPYRGT. 1994) and AMBER (Weiner, S. J. et al., J. Am. Chem. Soc., 106:765-784, 1984; Kollman, P. A., University of California at San Francisco, Calif., USA. COPYRGT. 1994).

Specialised computer programs may also assist in the process of selecting fragments or chemical entities. These include:
1. GRID (Goodford, P. J., J. Med. Chem., 28:849-857, 1985). GRID is available from Oxford University, Oxford, UK.
2. MCSS (Miranker, A. et al., Proteins: Structure, Function and Genetics, 11:29-34, 1991). MCSS is available from Accelrys, Inc., Burlington, Mass., USA.
3. AUTODOCK (Goodsell D. S. et al., Proteins: Structure, Function, and Genetics, 8:195-202 1990). AUTODOCK is available from Scripps Research Institute, La Jolla, Calif., USA.
4. DOCK (Kuntz I. D. et al., J. Mol. Biol., 161:269-288, 1982). DOCK is available from University of California, San Francisco, Calif., USA.

Once suitable chemical entities or fragments have been selected, they are assembled into a single compound or complex. Assembly may be preceded by visual inspection of the relationship of the fragments to each other on the three-dimensional image displayed on a computer screen in relation to the structure coordinates of RSV-F. This is generally followed by manual model building using software such as Quanta or Sybyl.

Useful programs to aid the person skilled in the art in connecting the individual chemical entities or fragments include:
1. CAVEAT (Bartlett, P. A. et al, “CAVEAT: A Program to Facilitate the Structure-Derived Design of Biologically Active Molecules”. In Molecular Recognition in Chemical and Biological Problems, Special Pub., Royal Chem. Soc., 78, pp. 182-196,1989). CAVEAT is available from the University of California, Berkeley, Calif., USA.
2. 3D Database systems such as MACCS-3D (MDL Information Systems, San Leandro, Calif.). This area is reviewed in Martin, Y. C., J. Med. Chem., 35:2145-2154, 1992.
3. HOOK (available from Accelrys, Inc., Burlington, Mass., USA).

As is well known to the person skilled in the art, instead of proceeding to build a single compound or complex for the target site in a step-wise fashion, one fragment or chemical entity at a time as described above, inhibitory or other target-binding compounds may be designed as a whole or de novo. Methods for achieving such include:
1. LUDI (Bohm, H.-J., J. Comp. Aid. Molec. Design, 6:61-78, 1992). LUDI is available from Accelrys, Inc. Burlington, Mass., USA.
2. LEGEND (Nishibata, Y. et al., Tetrahedron, 47:8985, 1991). LEGEND is available from Accelrys, Inc. Burlington, Mass. USA.
3. LeapFrog (Tripos Associates, St. Louis, Mo., USA).

Other molecular modelling techniques may also be employed. See, eg, Cohen, N. C. et al., J. Med. Chem., 33: 883-894, 1990). See also, Navia, M. A. et al., Current Opinion in Structural Biology, 2:202-210, 1992).

Once a single compound or chemical complex has been designed or selected by the above methods, the efficiency with which that entity may bind to a target site may be tested and optimized by computational evaluation. For example, an effective entity will preferably demonstrate a relatively small difference in energy between its bound and free states (ie a small deformation energy of binding). Thus, the most efficient entities should preferably be designed with a deformation energy of binding of not greater than about 10 kcal/mole, and preferably, not greater than 7 kcal/mole. Further, some entities may interact with the target site in more than one conformation that is similar in overall binding energy. In those cases, the deformation energy of binding is taken to be the difference between the energy of the free entity and the average energy of the conformations observed when the entity binds to the target site.

A compound or chemical complex designed or selected so as to bind to a target site may be further computationally optimized so that in its bound state it would preferably lack repulsive electrostatic interaction with the target enzyme. Such non-complementary (eg electrostatic) interactions include repulsive charge-charge, dipole-dipole and charge-dipole interactions. Specifically, the sum of all electrostatic interactions between the entity or other entity and the target site, when the entity is bound to the target site, preferably make a neutral or favourable contribution to the enthalpy of binding.

Specific computer software is available in the art to evaluate compound deformation energy and electrostatic interaction. Examples of programs designed for such uses include: Gaussian 92, revision C (Frisch, M. J., Gaussian, Inc., Pittsburgh, Pa. USA. COPYRGT. 1992); AMBER, version 4.0 (Kollman, P. A., University of California at San Francisco, Calif., USA. COPYRGT. 1994); QUANTA/CHARMM; and Insight 11/Discover (Accelrys, Inc. Burlington, Mass. USA. COPYRGT. 1994). These programs may be implemented, for instance, using a Silicon Graphics O2 workstation or Intel CPU based Linux duster. Other hardware systems and software packages will be known to the person skilled in the art.

Once a compound or chemical complex has been optimally designed or selected, as described above, modifications may be made to, for example, improve or modify its binding properties. Thus, for a compound, substitutions may be made in some of its atoms or side groups. Generally, initial substitutions of this kind will be conservative, that is the replacement group will have approximately the same size, shape, hydrophobicity and charge as the original group. It should, of course, be understood that components known in the art to alter conformation should be avoided. Such substituted chemical compounds may then be analyzed for efficiency of fit to a specific RSV-F target site by the same computer methods described in detail above.

Another approach is the computational screening of small molecule databases for compounds or chemical complexes that can interact in whole, or in part, to a target site. In this screening, the quality of fit of such entities to the target site may be judged either by shape complementarity or by estimated interaction energy (see for example: Meng, E. C. et al., J. Comp. Chem., 13: 505-524, 1992).

### SUMMARY OF THE INVENTION

In a first aspect, the present invention provides a method for identifying a candidate anti-viral agent, said method comprising the steps of:
(i) generating a three-dimensional structure model of RSV-F protein or a portion thereof, wherein said structure model comprises the three-dimensional structure of a target site to which an anti-viral agent may interact and thereby inhibit the activity of said RSV-F protein; and
(ii) identifying a candidate anti-viral agent by designing or selecting a compound or chemical complex with a three-dimensional structure enabling interaction with said target site.

In a second aspect, the present invention provides a method for screening compounds and/or chemical complexes for a candidate anti-viral agent, said method comprising the steps of:
(i) generating a three-dimensional structure model of RSV-F protein or a portion thereof, wherein said structure model comprises the three-dimensional structure of a target site to which an anti-viral agent may interact and thereby inhibit the activity of said RSV-F protein; and
(ii) screening said compounds and/or chemical complexes to identify any compound(s) or chemical complex(es) having a three-dimensional structure which enables interaction with said target site.

In a third aspect, the present invention provides a method for modifying a candidate anti-viral agent to provide an agent with improved anti-viral activity, said method comprising the steps of:
(i) generating a three-dimensional structure model of RSV-F protein or a portion thereof, wherein said structure model comprises the three-dimensional structure of a target site to which the candidate anti-viral agent interacts and thereby inhibits the activity of said RSV-F protein; and
(ii) modifying the candidate anti-viral agent to provide an agent with a three-dimensional structure more favourable to interaction with said target site or an agent which may otherwise interact more strongly with the target site than the candidate anti-viral agent.

The methods of the first to third aspects of the invention permit the identification of candidate anti-viral agents and their possible development into anti-viral agents suitable for therapeutic use. Such candidate anti-viral agents and/or developed anti-viral agents preferably show inhibitory activity against at least RSV, but may also show inhibitory activity against other viruses with fusion proteins homologous to RSV-F protein such as all other known members of the Paramyxoviridae family of viruses. These viruses may be pathogenic to species other than humans such as Bovine RSV infecting cattle and NDV infecting poultry. Viral-inhibitory activity shown by candidate anti-viral agents and/or developed anti-viral agents is caused, preferably, by an ability to bind to the said target site and thereby inhibit fusion activity of RSV-F protein to block cellular invasion and/or cell to cell transfer of the virus.

The three-dimensional structure model generated in step (i) of each of the above methods comprises, at least, the three-dimensional structure of a target site to which a candidate anti-viral or developed anti-viral agent may interact (eg bind) to inhibit the activity of RSV-F protein. Preferably, the target site comprises all or part of each of the following amino acids of RSV-F protein; Tyr33, Cys37, Ser38, Ala39, Val40, Ser41, Lys42, Gly43, Leu48, Arg49, Thr50, Lys315, Leu316, His317, Thr318, Ser319, Pro320, Leu321, Cys322, Thr323, Ser330, Asn331, Ile332, Cys333, Leu334, Thr335, Arg336, Thr337, Asp338, Arg339, Phe352, Pro353, Gln354, Ala355, Glu356, Thr357, Cys358, Phe366, Cys367, Asp368, Thr369, Met370, Asn371, Ser372, Leu373, Lys394, Ile395, Met396, Thr397, Ser398, Lys399, Thr400, Asp401, Val402, Ser403, Ser404, Ser405, Val406, Ile407, Thr408, Ser409, Leu410, Gly411, Ala412, Ile4113, Val414, Ser415, Lys419, Lys421 and Asp440 (as derived from the amino acid sequence of F protein from RSV strain A2 provided SEQ ID NO:1), or the corresponding amino acids of F protein from variant RSV strains (eg a known RSV mutant which includes Leu in place of Ser at position 398; Andries, K. et al., AntiviralRes., 50: A29, 2001). The atomic coordinate data for those amino acids within the three-dimensional structure model of RSV-F protein is provided in Table 1 hereinafter. Thus, the three-dimensional structure model generated in the methods of the first to third aspects is preferably generated using at least the atomic coordinate data of Table 1. However, more preferably, the three-dimensional structure model is generated from the atomic coordinate data of Table 2.

The methods of the first to third aspects of the invention are preferably in silico methods.

In the method of the first aspect, the step of identifying a candidate anti-viral agent (ie step (ii)) may be achieved by methods described above for designing and selecting compounds or chemical complexes with three-dimensional structures that fit and interact with a target site.

The method of the first aspect may further comprise a step of assessing the deformation of energy of the candidate anti-viral agent when brought from the free state to the target site-interacting state (eg bound state). Preferably, the deformation of energy is not greater than 10 kcal/mole and, more preferably, not greater than 7 kcal/mole. Additionally or alternatively to the step of assessing the deformation of the candidate anti-viral agent, the method of the first aspect may comprise a step assessing the enthalpy of the interaction (eg binding) of the candidate anti-viral agent with the target site. Preferably, the candidate anti-viral agent shall make a neutral or favourable contribution to the enthalpy of the interaction.

In the method of the second aspect, the step of screening compounds and/or chemical complexes to identify any compound(s) or chemical complex(es) with a three-dimensional structure enabling interaction with the target site (ie step (ii)) may be achieved by methods described above. The screened compounds and/or chemical complexes may belong to a library or database of suitable compounds and/or chemical complexes (eg ACD-SC (Available Chemicals Directory Screening Compounds), MDL Inc., San Leandro, Calif. USA.).

In the method of the third aspect, the step of modifying a candidate anti-viral agent (ie Step (ii)) may be achieved by methods described above such as substituting one or more groups (eg functional groups) on compounds.

In a fourth aspect, the present invention provides a computer for producing a three-dimensional structure model of RSV-F protein or a portion thereof, said structure model comprising the three-dimensional structure of a target site to which an anti-viral agent may interact and thereby inhibit the activity of said RSV-P protein, wherein said computer comprises:
(i) a machine-readable data storage medium comprising the atomic coordinate data of the following amino acids of RSV-F protein; Tyr33, Cys37, Ser38, Ala39, Val40, Ser41, Lys42, Gly43, Leu48, Arg49, Thr50, Lys315, Leu316, His317, Thr318, Ser319, Pro320, Leu321, Cys322, Thr323, Ser330, Asn331, Ile332, Cys333, Leu334, Thr335, Arg336, Thr337, Asp338, Arg339, Phe352, Pro353, Gln354, Ala355, Glu356, Thr357, Cys358, Phe366, Cys367, Asp368, Thr369, Met370, Asn371, Ser372, Leu373, Lys394, Ile395, Met396, Thr397, Ser398, Lys399, Thr400, Asp401, Val402, Ser403, Ser404, Ser405, Val406, Ile407, Thr408, Ser409, Leu410, Gly411, Ala412, Ile413, Val414, Ser415, Lys419, Lys421 and Asp440 (as derived from the amino acid sequence shown as SEQ ID NO:1), or the corresponding amino acids of F protein from variant RSV strains;
(ii) a working memory for storing instructions for processing said atomic coordinate data contained on the machine-readable data storage medium;
(iii) a central processing unit coupled to said working memory and to said machine-readable data storage medium for processing said atomic coordinate data to generate said three-dimensional structure model; and
(iv) a display coupled to said central processing unit for displaying a representation of said three-dimensional structure model.

Preferably, the machine-readable data storage medium (eg a magnetic or optical storage medium such as a hard drive, floppy disc or a CD-ROM) comprises the atomic coordinate data of Table 1 or Table 2.

The computer may further comprise:
(v) means for receiving and storing atomic coordinate data for a range of chemical components and substituents, wherein the central processing unit is capable of interacting with said receiving and storing means and selects from said range of chemical components and substituents suitable chemical components and substituents to assemble a compound or chemical complex which, based upon a three-dimensional structure generated by said central processing unit, a representation of which may be provided on said display simultaneously with the representation of said three-dimensional structural model of RSV-F protein or a portion thereof, is capable of interaction with said target site; and/or
(vi) means for receiving and storing atomic coordinate data for a range of compounds and/or chemical complexes, wherein the central processing unit is capable of interacting with said receiving and storing means to generate a three-dimensional structure for a compound or chemical complex selected from the range of compounds and/or chemical complexes, provide a representation of said three-dimensional structure on said display simultaneously with the representation of said three-dimensional structural model of RSV-F protein or a portion thereof, and thereby enable an assessment of whether said selected compound or chemical complex is capable of interaction with said target site.

The atomic coordinate data for the range of chemical components and substituents and the atomic coordinate data for the range of compounds and/or chemical complexes, can be obtained from suitable databases.

In a fifth aspect, the present invention provides a machine-readable data storage medium comprising the atomic coordinate data of the following amino acids of RSV-F protein; Tyr33, Cys37, Ser38, Ala39, Val40, Ser41, Lys42, Gly43, Leu48, Arg49, Thr50, Lys315, Leu316, His317, Thr318, Ser319, Pro320, Leu321, Cys322, Thr323, Ser330, Asn331, Ile332, Cys333, Leu334, Thr335, Arg336, Thr337, Asp338, Arg339, Phe352, Pro353, Gln354, Ala355, Glu356, Thr357, Cys358, Phe366, Cys367, Asp368, Thr369, Met370, Asn371, Ser372, Leu373, Lys394, Ile395, Met396, Thr397, Ser398, Lys399, Thr400, Asp401, Val402, Ser403, Ser404, Ser405, Val406, Ile407, Thr408, Ser409, Leu410, Gly411, Ala412, Ile413, Val414, Ser415, Lys419, Lys421 and Asp440 (as derived from the amino acid sequence shown as SEQ ID NO:1), or the corresponding amino acids of F protein from variant RSV strains.

Preferably, the machine-readable data storage medium comprises the atomic coordinate data of Table 1 or Table 2.

In a sixth aspect, the present invention provides a candidate anti-viral agent identified in accordance with the method of the first or second aspect, or an anti-viral agent produced in accordance with the third aspect.

The agent of the sixth aspect may be used to prepare a medicament for treating a viral infection, especially an infection by RSV or another virus with a fusion protein homologous to RSV-F protein such as all other known members of the Paramyxoviridae family of viruses which may infect species other than humans.

Thus, in a seventh aspect, the present invention provides the use of the agent of the sixth aspect in the preparation of a medicament for use in treating a viral infection.

And, in an eighth aspect, the present invention provides a method of treating a viral infection in a subject, said method comprising administering a medicament comprising an agent of the sixth aspect.

Preferably, the viral infection treated by the medicament of the seventh aspect or the method of the eighth aspect, is an infection by RSV or another virus with a fusion protein homologous to RSV-F protein such as all other known members of the Paramyxoviridae family of viruses. Most preferably, the viral infection is a respiratory infection by RSV.

### DETAILED DISCLOSURE OF THE INVENTION

Studies of the mechanism of infection by RSV both in vitro and in animal models show that the RSV fusion protein (RSV-F) is of critical importance in both initial cellular invasion and the formation of syncytia. In order to develop an understanding of the mode of action of RSV-F at the molecular level, a homology model of the post-fusogenic RSV-F structure has been built, including residues 29 to 98 and 160 to 515 of the intact protein. The atomic coordinate data for this structure is provided in Table 2.

According to the homology model, the structure of the RSV-F trimer can be divided into three regions; an extended stalk (commonly referred to as the “core” region of the protein) consisting of a 6-membered helical coiled-coil, a relatively globular head region, characterised by an unusual central cavity connected to the exterior of the protein by symmetrically located radial channels and a single axial opening and the neck region, an interface between the head and core. While not wishing to be bound by theory, it is hereby proposed that the RSV-F protein sequesters very hydrophobic fusion peptides in the internal cavity of the head domain in the pre-fusogenic form of the protein, and that within the cavity are three, symmetrically positioned grooves that link the top of the central channel with each of the radial channels. It is considered that these grooves are ideal sites of interaction for the three fusion peptides in the pre-fusogenic form and it is shown hereinafter that, surprisingly, they are also the likely primary sites of binding for three known RSV-F fusion inhibitors of quite different molecular structure. The mechanism of action for these inhibitors, which to the best of the present inventors' knowledge has not been previously determined at the molecular level, is attributed to their ability to interfere with the fusion peptide function through binding within the groove. Again, while not wishing to be bound by theory, it is considered likely that the inhibitors either prematurely displace the fusion peptides from sequestration in the head region of the protein prior to attachment of the F protein to a cell, or prevent egress of the fusion peptides once the F protein has bound to a cell. This, at a minimum, reduces the ability of the virus to infect cells or form syncytia and may also result in the aggregation of viral particles due to the premature exposure of extensive hydrophobic regions of the fusion protein.

This information provides a rational basis for the design and development of compounds and/or chemical complexes that strongly interact (eg bind with high affinity) to one or more of the abovementioned grooves (Site As) in RSV-F and thereby disrupt RSV-F function. Accordingly, these compounds are likely to have anti-viral activity and thereby therapeutic value in the treatment or prevention of viral infections such as a respiratory infection by RSV.

Each of the three Site As of RSV-F protein are defined by the following amino acids: Tyr33, Cys37, Ser38, Ala39, Val40, Ser41, Lys42, Gly43, Leu48, Arg49, Thr50, Lys315, Leu316, His317, Thr318, Ser319, Pro320, Leu321, Cys322, Thr323, Ser330, Asn331, Ile332, Cys333, Leu334, Thr335, Arg336, Thr337, Asp338, Arg339, Phe352, Pro353, Gln354, Ala355, Glu356, Thr357, Cys358, Phe366, Cys367, Asp368, Thr369, Met370, Asn371, Ser372, Leu373, Lys394, Ile395, Met396, Thr397, Ser398, Lys399, Thr400, Asp401, Val402, Ser403, Ser404, Ser405, Val406, Ile407, Thr408, Ser409, Leu410, Gly411, Ala412, Ile413, Val414, Ser415, Lys419, Lys421 and Asp440 of the monomers of RSV-F protein. It is envisaged that homologous fusion proteins from other viruses (eg other viruses from the family Paramyxoviridae) may include a site similar to Site A which may also be useful for identifying or screening anti-vital agents. Thus, it is to be understood that the present invention also extends to the generation of three-dimensional structure models of homologous fusion proteins (especially those showing at least 20%, preferably at least 30%, more preferably at least 50%, and most preferably at least 80% (as calculated using the CLUSTAL algorithm; Thompson et al., Nucleic Adds Research, 24:4876-4882,1997), amino acid similarity to the amino acid sequence of RSV-F protein from RSV strain A2, and/or which have an amino acid backbone with a root mean square deviation from the amino acid backbone of RSV-F protein of not greater than 1.5 Å), and the use of such three dimensional structure models to identify and screen for candidate anti-viral agents.

Agents resulting from the use of a method of the first to third aspects of the present invention, may be assayed for anti-viral activity by a number of in vitro assays. For example, the agents can be assayed for their effect on viral activity by culturing virus in the presence of said agents. The efficacy of an agent will be reflected in the reduction of viral infectivity in the presence of increasing levels of said agent. Analysis of in vitro anti-viral activity of the agents can also be carried out using a recombinant RSV-F fusion assay in which suitable mammalian cells (eg 293 cells) are transfected with plasmids or vectors carrying a polynucleotide sequence for expressing RSV-F protein. Expression of the RSV-F protein results in cellular fusion and the formation of syncytia, which effect may be suppressed by an agent which is an inhibitor of RSV-F protein activity. The relative levels of syncytia formation in the presence of increasing levels of an inhibitor provides an assessment of the efficacy of the agent.

Medicaments comprising anti-viral agents may be prepared in accordance with methods and materials (eg excipients and carriers) well known to a person skilled in the art As such, the medicament may be formulated for various routes of administration including oral, nasal, vaginal, rectal, topical, intramuscular and intravenous routes.

Oral formulations may be in the form of tablets, capsules or emulsions, and may include one or more carriers such as lactose, saccharose, starch, calcium stearate and methylcellulose. Alternatively, oral formulations (and nasal formulations) may be in the form of a gas or aerosol for inhalation (particularly suitable for treatment of respiratory infections). Vaginal and rectal formulations may be in the form of pessaries and suppositories respectively. Topical formulations may be in the form of ointments, creams or lotions. Injectable formulations for administration by intramuscular or intravenous routes may include well known carriers and/or excipients.

It is routine for a person skilled in the art to determine an effective dosage of an anti-viral agent and he/she will understand that the amount may vary according to, for example, the type of virus to be treated and the severity of the infection. However, generally a medicament comprising an anti-viral will be administered to a subject to provide a dose of the anti-viral agent in the range of 0.1 to 10 mg/kg/day.

The homology model of the post-fusogenic RSV-F structure described herein may also be used for the rational selection of regions within the RSV-F protein which, when appropriately presented, may be antigenic and capable of eliciting RSV-specific antibodies which may be useful for, for example, RSV diagnostic assays and/or methods of prevention or treatment of RSV infection.

Thus, the present invention also relates to a method of identifying a region within the RSV-F protein comprising an antigenic determinant which may be used to elicit antibodies capable of binding to RSV, said method comprising the steps of:
(i) generating a three-dimensional structure model of RSV-F protein; and
(ii) identifying from said structure model a surface-located region of the RSV-F protein which may include an antigenic determinant.

The three-dimensional structure model used to identify a region within the RSV-F protein comprising an antigenic determinant capable of eliciting antibodies capable of binding to RSV, is preferably generated from the atomic coordinate data of Table 2.

The identification of a surface-located region of the RSV-F protein which may include an antigenic determinant may simply be made by the eye of a person skilled in the art or, in a preferred in silico method, the identification may be made a computer. The surface-located region will typically comprise 5 to 100 amino acids, more preferably 10 to 50 amino acids. The amino acids will preferably be contiguous, however the present invention also contemplates non-linear antigenic determinants comprising non-contiguous amino acids. A “surface-located” region of the RSV-F protein is to be regarded as consisting of any region of the RSV-F protein which is accessible and may be bound to by an anti-RSV-F protein.

The antibodies elicited by the identified region preferably binds to RSV in a specific manner (ie the antibodies show negligible, if any, binding to other agents). The elicited antibodies may be RSV-neutralising antibodies (ie antibodies able to inhibit the infectivity of RSV).

An identified surface-located region of the RSV-F protein may be tested for its ability to elicit antibodies capable of binding to RSV by any of the standard methods known to a person skilled in the art For example, the surface-located region may be isolated or synthesised, conjugated to a suitable carrier protein (eg bovine serum albumin), and used to raise monoclonal antibodies by routine methods. The elicited monoclonal antibodies may then be tested using, for example, an appropriate ELISA assay to determine whether the monoclonal antibodies bind to RSV.

The capacity of raised monoclonal antibodies to neutralise RSV may be assessed by conducting standard virus neutralisation tests in vivo using susceptible animals, or otherwise, in tissue cultures.

Thus, the method of identifying a region within the RSV-F protein comprising an antigenic determinant which may be used to elicit antibodies capable of binding to RSV, may additionally comprise the steps of:
(iii) producing and isolating antibodies elicited by said surface-located region of RSV-F protein; and
(iv) assessing said antibodies for RSV neutralising capacity.

The present invention further relates to RSV specific antibodies or fragments thereof (eg Fab, Fc or scFv fragments), which bind to a region of the RSV-F protein comprising a novel antigenic determinant identified by the abovementioned method. Where such antibodies or fragments thereof are capable of neutralising RSV, the antibodies or fragments thereof may be used as an anti-viral agent, especially against an infection by RSV or another virus with a fusion protein homologous to RSV-F protein such as all other known members of the Paramyxoviridae family of viruses. Therefore, the present invention also relates to a medicament comprising such antibodies and its use in a method of treating or preventing a viral infection in a subject. Antibodies in accordance with the present invention may also be used in methods to detect RSV or another virus with a fusion protein homologous to RSV-F protein (eg all other known members of the Paramyxoviridae family of viruses), such as diagnostic assays for subjects suspected to be suffering from RSV infection.

Moreover, the present invention relates to the use of novel antigenic determinants identified from the three-dimensional structure model of RSV-F protein, as vaccine agents (optionally in combination with a suitable adjuvant or carrier) which may be administered with a suitable adjuvant or carrier to a subject to treat or prevent a viral infection. Alternatively, suitable recombinant viral vectors may be engineered, by routine methods, to express an antigenic determinant identified from the three-dimensional structure model of RSV-F protein. Such viral vectors may be administered with a suitable adjuvant or carrier to a subject to treat or prevent a viral infection. Suitable viral vectors are well known to a person skilled in the art, and include vaccinia and adenovirus vectors.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

The present invention is further described below with reference to the accompanying figures and following, non-limiting examples.

### BRIEF DESCRIPTION OF THE ACCOMPANYING FIGURES

FIG. 1: Sequence alignment of RSV-F, the RSV-F core and NDV-F used for model building. Regions shaded grey in NDV-F were not present in the crystal structure. Regions of the RSV-F sequence that are shaded black were modelled on the structure of the RSV-F core rather than on the structure of NDV-F, where it was present. Grey shading of the RSV-F sequence indicates regions that are absent from the modelled structure.

FIG. 2: Structures of the three known RSV-F inhibitors used as probes to identify Site A in the RSV-F head domain. (A) COMPOUND 1, (B) COMPOUND 2, (C) COMPOUND 3.

FIG. 3: Structure of the final RSV-F model. (A) Full heavy-atom representation and (B) secondary structure schematic cartoon of the protein.

FIG. 4: (A) Structure of RSV-F with the best two COMPOUND 1 docking results for each of the sub-divisions of the protein used in defining Site A. (B) The top 1% of all the docking results for COMPOUND 1. These dearly duster in the head region of the protein.

FIG. 5: (A) Structure of RSV-F with the best two COMPOUND 2 docking results for each of the subdivisions of the protein used in defining Site A. (B) The top 1% of all the docking results for COMPOUND 2. These dearly duster in the head region of the protein.

FIG. 6: (A) Structure of RSV-F with the best two COMPOUND 3 docking results for each of the subdivisions of the protein used in defining Site A. (B) The top 1% of all the docking results for COMPOUND 3. These dearly duster in the head region of the protein.

FIG. 7: Stereo diagrams of the best docked results for COMPOUND 1 (top) COMPOUND 2 (centre) and COMPOUND 3 (bottom). Each best structure is found within the Site A groove.

FIG. 8: Diagrammatic representation of the position of the three symmetrically related Site As in the structure of RSV-F. The closest atoms of any two ligands docked in these sites are approximately 15 Å apart. (A) Side-on view of RSV-F showing approximate position of one Site A. (B) Top-down view showing relative positions of the three symmetrical Site As.

FIG. 9: Details of one of the Site A structures of RSV-F. (A) Stereo view of residues of Site A are shown in ball and stick representation with the rest of RSV-F shown as a backbone tube in grey. (B) Stereo figure showing the solvent accessible surface of Site A shaded by polarity of the residue (white, hydrophobic; grey polar; dark grey charged). The shape of the Site—a deep hydrophobic groove with flanking polar and charged residues—is very clear in this view.

FIG. 10: Diagrammatic representation of the RSV-F structure model with the known antigenic sites (ie sites I, II, IV, V and VI) on the RSV-F protein shown. Residues that give rise to antibody resistant mutants, and hence define each site, are shown as space-filling models. Site I, residue 398; Site II, residues 262, 268, 272 and 275; Sites IV, V and VI overlap and include residues 429, 432, 433, 436 and 437. The sites of two overlapping peptide regions of RSV-F, ie Peptides I and II, which react with neutralising sera from RSV infected animals and induce neutralising antibodies to RSV are also shown.

### EXAMPLE 1

### Materials and Methods

Homology Modelling of RSV-F

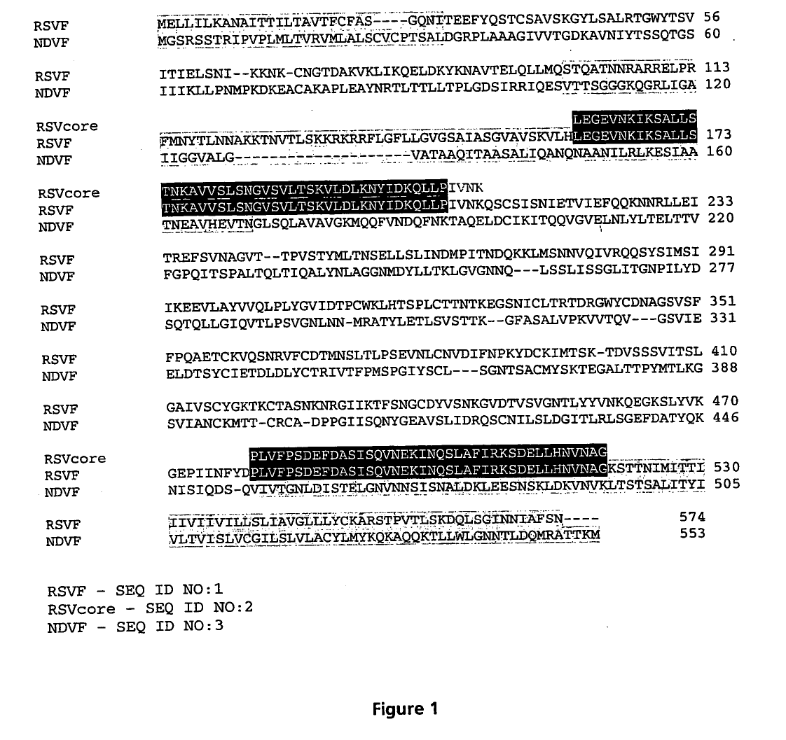

As a preliminary step in modelling the RSV-F protein, a chimeric construct of the NDV-F structure and the RSV-F core structure was produced. This chimera was built using SwissPDB Viewer (SPDBV) (Guex, N. and Peitsch, M. C., Electrophoresis, 18:2714-2723, 1997) by superimposing the coordinates of G184-Q202 of chain A from the RSV-F core onto G171-Q189 of chain A in the NDV-F structure. These residues were selected after conducting alignment of the amino acid sequence of the two proteins from which it was determined that they were structurally equivalent regions (FIG. 1). The two structures showed an extremely good “fit”. Due to this excellent correspondence in structure, completion of the chimera by manual editing of the combined PDB files was straightforward. The chimeric structure was subject to energy minimisation of the 4 residue stretches either side of the six joining points to remove any minor errors that the manual combination of structures had introduced. This was relatively unimportant for the HR-A helix, as this region had matched so well. The distance from the last residue in the NDV-F to the first of the RSV core for HR-B was, however, somewhat too long and required minimisation to remove the problem.

The chimera was then used as the basis for building a model of RSV-F. The primary alignment used for model building was created using Clustal (Thompson et al., Nucleic Adds Research, 24: 4876-4882, 1997) and was adhered to except for one loop region in F2 (FIG. 1). Note that the sequence used for RSV-F corresponds to the RSV strain A2 clone in use in the Biota Virology Laboratory and differs in 5 sites (E66K, P101Q, P102A, 1379V and M447V) from the GenBank entry AAB59858.1. A single loop (residues A276 to S289 in RSV) was not able to be built in a satisfactory manner using the alignment and was manually adjusted on the basis of PHD secondary structure prediction and the maintenance of a conserved disulfide bond, with the final alignment shown in FIG. 1. The two chains of each F1-F2 pair were modelled as separate entities in SPDBV, with manual inspection of the candidate loops for each mismatch used to select the optimal structure. A total of 10 loop structures were rebuilt (1 in F2 and 9 in F1).

After insertion of the required loops, the separate RSV-F1 and RSV-F2 structures were combined and the complete trimeric RSV-F structure formed by superimposing the initial dimer onto the coordinates of the other dimers in the chimera. Minimisation of the complete RSV-F model was then carried out with Discover in the InsightII package (Accelrys) under the cvff forcefield to remove undesirable interactions that had been generated by the modelling process. Firstly, the residues surrounding each inserted loop were minimised with the rest of the structure held fixed to correct any minor mismatches at insertion points. Then the backbone atoms of the whole protein were held fixed and the sidechain atoms minimised using Steepest Descent minimisation (see the InsightII Manual distributed with the software and references therein, all of which are to be regarded as incorporated herein by reference) until the maximum gradient value dropped to below 10. The backbone constraints were then removed and the whole structure allowed to minimise for a further 100 steps of Steepest Descent minimisation.

Ligand Docking

Consideration of the intricate surface and three-fold symmetry of the RSV-F structure suggested that there would be two classes of potential target sites for small molecules: 1) sites where ligand binding can occur simultaneously in three non-overlapping symmetrical places on the protein; 2) sites at points where symmetrically equivalent interactions overlap, meaning each trio of sites could only be occupied by a single ligand at any time, but in one of three potential orientations. This complication, and the very extensive surface area of the protein (more than 40,000 Å²), indicated that locating the appropriate binding site for RSV-F inhibitors would not be trivial and would require care to ensure that the site is defined as objectively as possible. Subsequently, a two-stage docking analysis, described in detail below, was used with three different RSV-F inhibitors to try and identify sites of action for different classes of RSV-F ligands.

Docking analysis of RSV-F was carried out using the program AutoDock 3.0.3. The algorithm employed by AutoDock is described in detail in the AutoDock User Guide distributed with the software and references therein (Morris, G. M. et al., J. Comp. Chem., 19: 1639-1662, 1998), all of which are to be regarded as incorporated herein by reference. In brief, the region of space around the protein target selected for analysis is divided into a grid of points spaced a user-definable distance apart. At each of these points, the potential energy of every type of atom in the ligand being docked is calculated and stored in a potential map. During the actual docking process, the ligand is inserted into the assigned region of space and translated, rotated and allowed to alter user-selected torsion angles and the energy of the ligand rapidly calculated by reference to the stored maps of potentials, rather than direct calculation during the docking itself. After a number of steps of optimisation determined by the user, the best conformation is saved and the process repeated. After the desired number of conformations are generated they are grouped into “clusters” of similar structures, defined by a user selected RMS (Root Mean Square deviation in atomic position) tolerance.

For docking with RSV-F, two different levels of docking analysis were used. A relatively coarse docking analysis over the entire protein was carried out first to aid in identifying potential binding sites on the protein without the intention of defining details of the interaction at such sites. This analysis, was partly automated by a simple set of in-house scripts that take, as input, a coordinate file for a protein in PDB (protein data bank) format then define a set of overlapping cubic regions that completely encompass the protein surface. These regions are then used for separate docking analyses using AutoDock. Each region was defined as a cube of 121×121×121 grid points separated by a grid step of 0.5 Å, making a cube of 60.5 Å each side. The cubes overlapped by 10 Å, to ensure adequate coverage of every point on the protein surface. Typically, a total of 16 cubes was needed for complete coverage of the RSV-F surface. A total of 50 docked conformations was calculated for each cube, with the conformations clustered using an RMS tolerance of 1.8 Å. Thus for each ligand used for full-protein docking runs, 800 docked conformations were calculated.

The top 1% of docked conformations (as ranked by the docking algorithm's calculated energy for the best structure in each cluster) were subjected to detailed visual inspection. Of these eight conformations, 4 were selected as candidate physiological binding sites on the basis of the interactions observed and the position of the site on the protein surface. These candidate sites were then used as the centre of detailed docking analyses using a higher-resolution grid (61×61×61 points, 0.375 Å grid step size for a grid 22.9 Å each side, 100 structures calculated and clustered with a tolerance of 1.2 Å). Visual analysis of the best structures in each site was then used to discriminate the most probable site of physiological ligand binding.

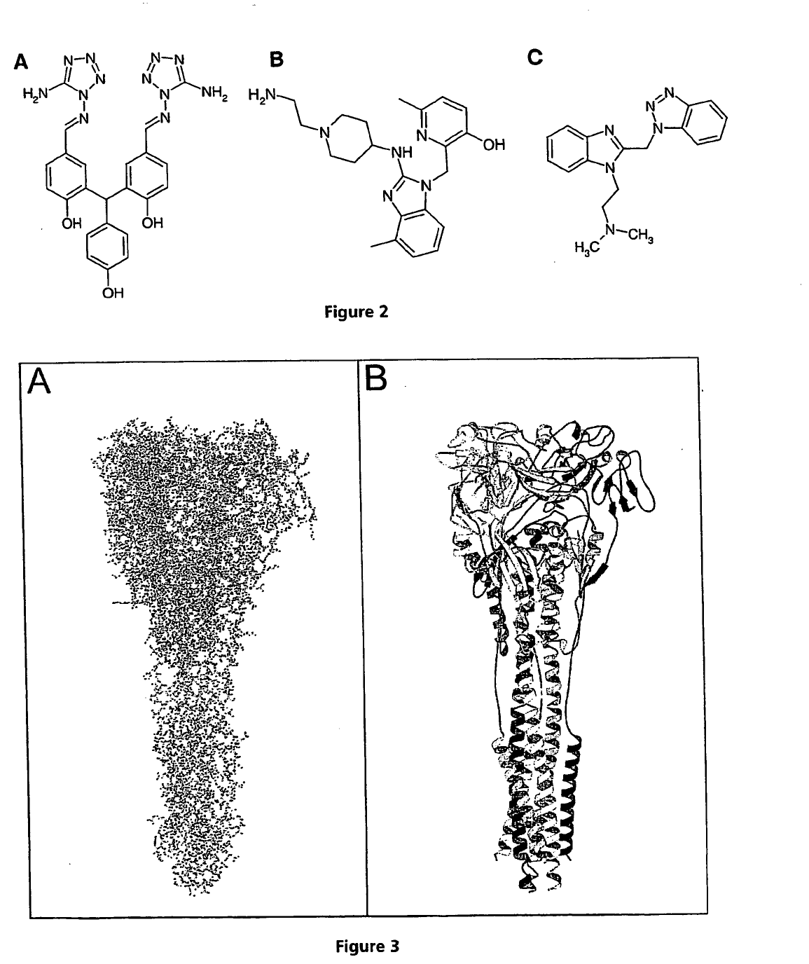

Three published RSV-F inhibitors with quite different molecular structures were submitted to the docking regime described above. The first is a compound in development by the company Viropharma, described in International patent specification No. WO 99/38508 (FIG. 2A) and referred to herein as COMPOUND 1. The second compound is being developed by Jannsen Pharmaceuticals and described in International patent specification No. WO 01/00612 (FIG. 2B) and referred to herein as COMPOUND 2. The last compound used as a probe to identify potential sites of inhibitor action is described by Bristol-Myers Squibb in U.S. Pat. No. 4,619,942 (FIG. 2C) and referred to herein as COMPOUND 3.

Binding Site Characterisation

The conformations determined by AutoDock do not take flexibility of the protein into account. Subsequently, to optimise the accuracy of the docked structures, the ligand and all residues within 15 Å of the ligand centroid were subject to energy minimisation using Discover (Accelrys, USA). Details of the contacts made between the ligand and protein were measured using a simple in-house script that measures the distance between every atom in the ligand and the atoms in the neighbouring residues of the protein then reports those atoms separated by less than a user-defined cutoff value. This data was then interpreted to derive two pieces of information; 1) delineation of the residues that made up the binding site for the particular docked conformation of the ligand; 2) a list of potential sites for mutagenesis studies useful for the selection of the actual physiological binding site.

Viral Assays: Assay for Compounds Active Against RSV

Graded concentrations of test compounds were prepared by serial dilution in 96 well tissue culture plates containing 100 μL per well of assay media (Eagles Minimal Essential Medium (MEM) supplemented with 2% foetal bovine serum and Glutamine alanyl-L-glutamine). Human laryngeal carcinoma (HEp2) cells were trypsinised and diluted in assay media to a concentration of 1.5×10⁵ viable cells/mL according to standard methods. 1.5×10⁴ of the diluted cells were added to several individual wells of each 96 well plate which were designated uninfected control wells. The remaining cell suspension was exposed to infectious RSV by addition of sufficient virus to the suspension to yield a concentration of 0.4 plaque forming units per cell. The cell-virus suspension was mixed well by inversion and 100 μL immediately dispensed into the remaining wells of the 96 well plates. Assay plates were incubated in a 37° C. incubator for 5 days. Viral replication was quantified by determination of cell viability substantially as described by Watanabe and colleagues (Watanabe, W. et al., J. Virol. Methods, 48: 257-265, 1994). Briefly, 100 μL of 3 mg/mL MTT (3(4,5-dimethylthiazol-2-yl)-2,5-diphenyltetrazolium bromide) was added to each well and plates incubated at 37° C. in a 5% CO₂ incubator for 2 hours. Wells were aspirated to dryness and the crystalline product of MIT metabolism dissolved by addition of 200 μL of 100% isopropanol. The extent of metabolism was measured via determination of absorbance using a computer-controlled microplate reader at 540 nm with a reference wavelength of 690 nm. The metabolism of MTT is proportional to the number of viable HEp2 cells (Watanabe et al., supra). The 50% cytotoxic concentration (CC₅₀) and the 50% effective concentration (EC₅₀) were determined using computer curve fit software (eg XLFit® (ID Business Solutions, Guildford, UK) and Ascent® (MTX Lab Systems, VA, USA)).

Viral Assays: Selection of Drug Resistant Virus and Cross-Resistance Testing

RSV was grown in the presence of graded concentrations of COMPOUND 1 or COMPOUND 2 diluted in assay medium. Putative resistant virus (evident by the presence of viral cytopathology in these cultures) was recovered, RNA isolated and the F gene sequence determined via RT-PCR according to standard methods. Changes in nucleotides resulting in amino acid sequence changes were noted and the respective amino acids were mapped on to the known F protein sequence. In addition, resistant viruses were grown, titred, F gene re-sequenced and assayed for drug resistance using the Assay described above.

Syncytia Formation Assays

293 cells (Graham, F. L. et al., J. Gen. Virol. 36: 59-72, 1977) were transfected with plasmid pCICO F.FL.opt which contains a gene encoding RSV-F. Cells transfected with this plasmid were incubated at 37° C. in a 5% CO₂ incubator and observed 24-48 hours post transfection. The F-transfected cells are identical in appearance to RSV-F infected cells, being characterised by the presence of many large syncytia and dying cells due to the fusion of cellular membranes by the expressed F protein. Control cells were confluent at the same time point. Fusion inhibitors were assayed by transfecting the plasmid into 293 cells as above in the presence of varying amounts of inhibitors. Syncytia were scored by microscopic inspection and cell viability determined using MTS (3(4,5-dimethylthiazol-2-yl)-5-(3-carboxymethoxyphenyl)-2-(4-sulfophenyl)-2H-tetrazolium, Promega) via standard methods. The reduction in scorable syncytia formation and cell viability was used as a direct measurement of the efficacy of the inhibitor used. Site specific mutants of RSV-F were produced (see below) and assayed in an identical manner.

Mutagenesis

Mutagenesis was used to introduce specific amino acid changes in recombinant RSV-F using the pCICO F.FL.opt plasmid carrying the optimised wild-type RSV-F gene and the synthetic oligonucleotide PCR method. Where necessary, particularly with mutant constructs that showed poor expression in the syncytia formation assay, the entire modified RSV-F gene was sequenced to ensure that only the desired mutations had been introduced into the sequence.

Results

Molecular Modelling

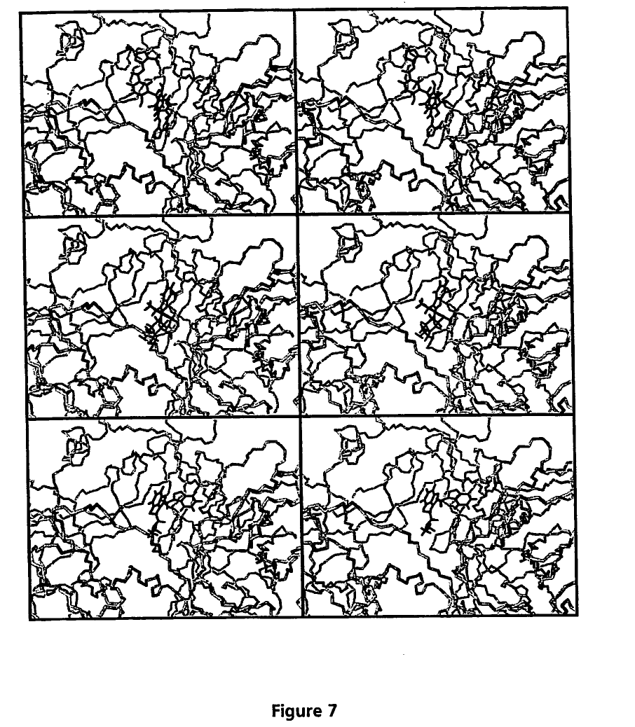

The final modelled structure of RSV-F residues 29 to 98 and 160 to 515 is shown in FIG. 3. The protein consists of a trimer of disulphide-linked F2-F1 pairs arranged with three-fold symmetry about a central axis. As described in the published work detailing the structure of the related viral fusion protein NDV-F (Chen, L. et al., supra) the protein can be divided on inspection into three regions. The base is an elongated stalk, consisting primarily of a 6-membered helical coiled-coil and containing the two helical-repeat regions identified in the RSV-F sequence. The head region, approximately the shape of an inverted triangular prism, has a large central cavity accessible to the exterior of the protein through a channel down the axis of the protein and three symmetrically positioned radial channels. Between these two sections of the protein is an interface described as the neck region. Chen and colleagues (Chen, L. et al., supra) noted that this structure had obvious implications for a possible mode of action of RSV-F (see FIG. 7, Chen, L. et al., supra), wherein the fusion peptides at the end of HR-1 are sequestered in some undefined manner within the head of the protein, only to be released when contact with a target cell triggers the exposure of the fusion peptides which then bury within the target cell membrane. According to their proposal, a rearrangement of the conformation of the F protein then brings the fusion peptides and viral anchor peptides into dose proximity, simultaneously resulting in the fusion of the two membranes. They were unable, however, to speculate on any of the details of the interactions between the sequestered fusion peptide and the inner surface of the cavity as they lacked data that could aid in pin-pointing sites of contact between the two. The invention described herein provides a detailed description of the site of interaction.

Docking of RSV-F Inhibitors

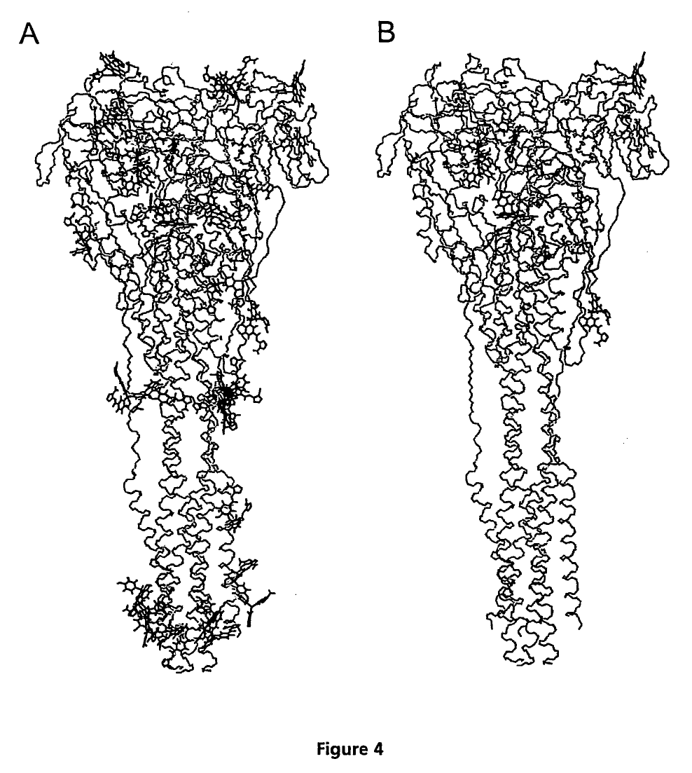

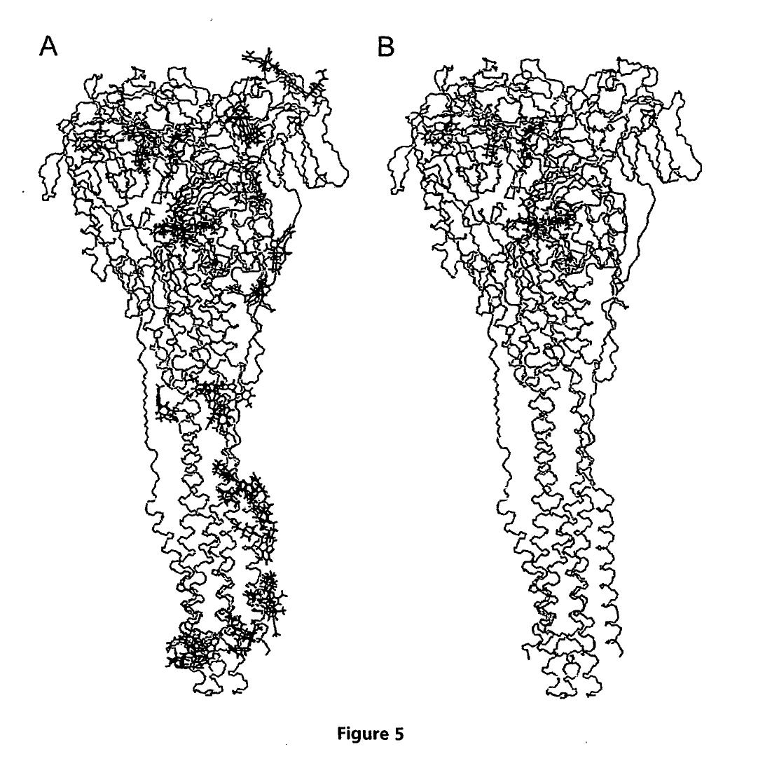

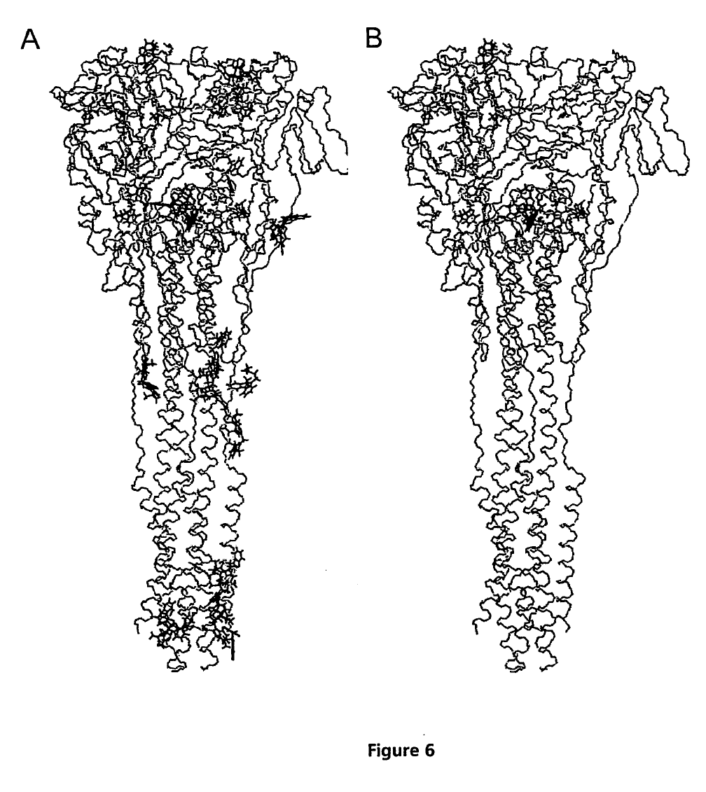

The docking results for the three different inhibitors are summarised diagrammatically in FIGS. 4, 5 and 6. These show (A) the best two clusters for each cube in the coarse dock and (B) the best 8 clusters in the high-resolution dock for molecules COMPOUND 1, COMPOUND 2 and COMPOUND 3, respectively. It is immediately obvious that the best docking results are restricted to sites in and around the head domain of the protein. This is perhaps unsurprising as the coiled-coil structure of the stalk is relatively free of conformational features that might allow extensive contacts between ligands and the protein, while the head region has many convolutions and cavities that would promote such interactions.

While the docking analysis described above was carried out in a unbiased manner, there are some published data concerning the residues that may be involved in interaction with two of the known RSV-F inhibitors. Viral escape mutants resistant to inactivation by COMPOUND 2 were reported by Andries and colleagues (Andries, K. et al., supra) and have been isolated by us using COMPOUND 1 and COMPOUND 2 in separate experiments. Andries reported that two residues in RSV-F were mutated in COMPOUND 2-resistant virus: S398 (to Leucine) and D486 (to Asparagine). In COMPOUND 1-resistant viral cultures F488 to Valine or Isoleucine were commonly isolated.

The results of the docking studies were correlated with this known resistance mutant data and it was with some surprise that all three compounds were found to dock best in a site immediately adjacent to the position of one of these escape-mutant residues, S398.

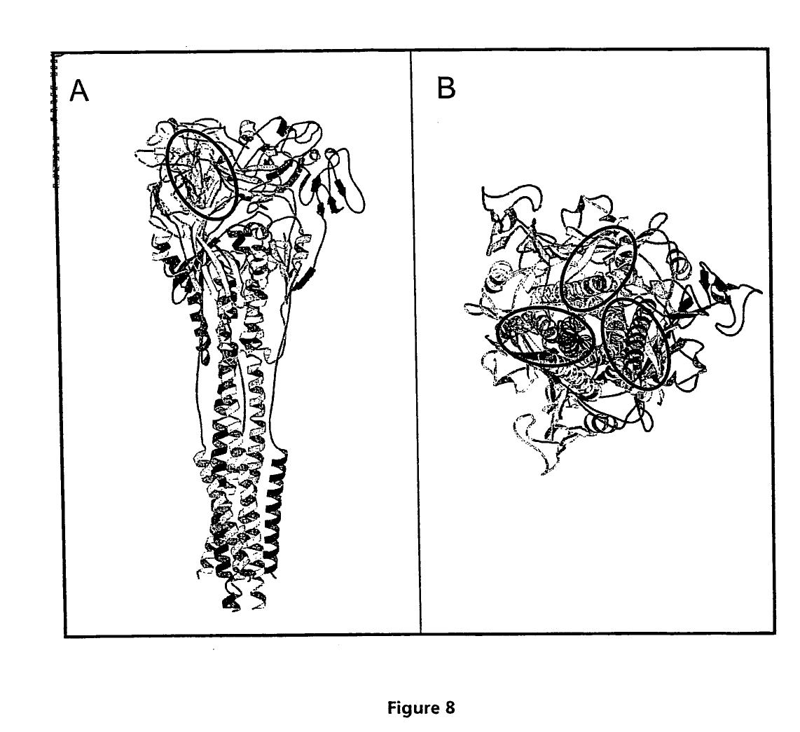

The best docked conformations for each of the three ligands are shown in FIG. 7. The details of the interactions are different, as the molecules are substantially different in their chemical structure, but it is clear that each is associated with a site that lies in a groove beneath S398. This region of the protein is inside the central cavity, with S398 itself at the very top edge of the cavity. The groove that appears to act as the primary binding site for all three anti-fusion compounds curves down from the edge of the axial channel to one of the three radial channels. There are three symmetrically related sites in each of the three sections of the trimer, separated from each other by approximately 15 Å (FIG. 8). Three ligands could therefore bind simultaneously to the protein at these sites. It should be noted that there is no published information regarding the stoichiometry of binding of any of the ligands to RSV-F so the actual ratio of binding required for inhibition of fusion is unclear.

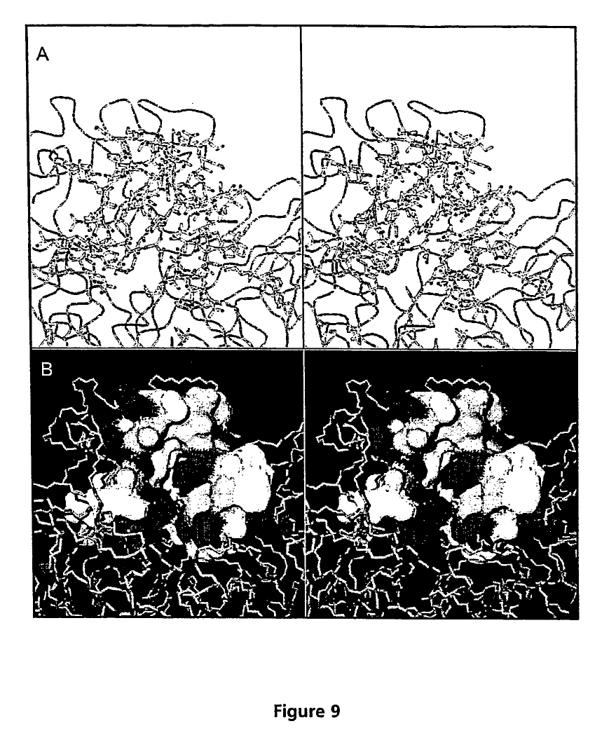

This groove was assigned the label Site A, such that there are three Site As in each RSV-F molecule. Visual inspection of Site A shows that it consists of a hydrophobic pocket and groove, with flanking polar residues including a series of potential salt-bridge pairs (FIG. 9). While the exact contacts vary considerably between the three ligands, the following residues from the three monomers in the RSV-F trimer can be included in a definition of each Site A: Tyr33, Cys37, Ser38, Ala39, Val40, Ser41, Lys42, Gly43, Leu48, Arg49, Thr50, Lys315, Leu316, His317, Thr318, Ser319, Pro320, Leu321, Cys322, Thr323, Ser330, Asn331, Ile332, Cys333, Leu334, Thr335, Arg336, Thr337, Asp338, Arg339, Phe352, Pro353, Gln354, Ala355, Glu356, Thr357, Cys358, Phe366, Cys367, Asp368, Thr369, Met370, Asn371, Ser372, Leu373, Lys394, Ile395, Met396, Thr397, Ser398, Lys399, Thr400, Asp401, Val402, Ser403, Ser404, Ser405, Val406, Ile407, Thr408, Ser409, Leu410, Gly411, Ala412, Ile413, Val414, Ser415, Lys419, Lys421 and Asp440. This list includes residues that make direct contact with any of the ligands as well as those residues which are immediately adjacent and which have a strong influence on the orientation and flexibility of the contact residues.

Confirmation of Site A

Having identified a single site that appears to be responsible for binding three very different types of fusion inhibitor, two separate approaches were employed to try and confirm the relevance of the site and thus the accuracy of the study as a whole. Firstly, a series of specific mutations were inserted into recombinant RSV-F and their effect on fusion activity and susceptibility to fusion inhibitors examined in a syncytia formation assay. The second approach was to screen RSV cultures for resistance to COMPOUND 1 and COMPOUND 2 to try and identify new escape mutants which could then be correlated with the proposed site.

A total of 14 mutations were suggested on the basis of the contacts observed in the docking experiments (summarised in Table 3). Nine of these suggested mutants were in Site A, the other five were at other points in the protein to act as site-definition controls. Some of the mutations were minor changes, such as serine to threonine, specifically intended to cause slight alterations in susceptibility of RSV-F to inhibitors without disrupting function. More drastic mutations, such as serine to leucine, were also proposed as changes guaranteed to have a substantial effect on inhibition, though probably also on activity. Of the 14 mutants, 8 were inactive in the assay apparently due to misfolding and degradation of the protein before it reaches the cell surface. Two of the active mutant proteins showed clear resistance to inhibitors. These were S319L (a Site A mutant) and T326L. The S319L mutant is fully active in the syncytia formation assay and almost completely resistant to inhibition by either COMPOUND 1 or COMPOUND 2, showing fusion activity at concentrations more than two orders of magnitude more than needed to cause complete inhibition in wild-type RSV-F. The resistance seen in T326L is weaker than that for S319L (about 10 fold only) and restricted to COMPOUND 1. T326L, however, is not part of the defined Site A, being more on the outside face of the head region. It is, however, in close proximity to some Site A residues (the closest, Ile395 which interacts only with COMPOUND 1, being just 3.8 Å away) and a substantial change in local conformation at T326 could be expected to influence these residues.

Isolation of escape mutants through viral assay has also produced supporting evidence for the relevance of Site A. Three mutants were isolated by culturing virus in the presence of COMPOUND 1. These are F488V, T400A and T337A, the latter two of which are in RSV-F Site A. The F488V mutant is a variation on the F488L/I mutants that have been identified elsewhere. The other two mutations are at the top of Site A, flanking the position occupied by the three ligands in their best docking sites. A double mutant D489Y, F488I has also been identified. This F488 mutation has been seen previously but it is the first identification of a mutant at D489. Interestingly, there is a known D486 resistance mutant for COMPOUND 2, suggesting that the aspartic add residues flanking F488 may have a role in stabilisation of the pre-fusogenic form of RSV-F where it is hypothesised that F488 interacts with Site A. A possible role for these aspartates is interaction with some of the positively charged residues that flank the Site A groove.

Two new COMPOUND 2 escape mutants have also been identified. One contains the same D489Y mutation mentioned above for COMPOUND 1 and another new mutation, L1381. Unfortunately L138 is outside the RSV-F model in F2, so it is difficult to comment on its potential role in RSV-F activity. The other COMPOUND 2 escape mutant is a triple mutant, containing the L138I substitution, H516N (which has been shown to have no effect on activity or resistance when recreated alone by mutagenesis of F and expression via the fusion assay—data not shown) and, most intriguingly, S319F. Thus, a major substitution at S319 correlates with COMPOUND 2 resistance in a viral background. The correlation between the escape mutations identified for COMPOUND 2 and COMPOUND 1 and cross-resistance assays provide strong support for their acting at the same site and in a very similar manner. Some of the resistance mutant isolation and cross-resistance data is summarised in Table 4.

These results provide dear evidence of cross-resistance to COMPOUNDS 1 and 2 via fusion peptide and stalk mutations. This strongly suggests an identical site and mechanism of action for both compounds.

The escape mutant data, therefore, supports the importance of Site A in the function of RSV-F and the action of RSV-F inhibitors. Identified mutants are located either within Site A itself, or in a duster well removed from the Site and associated with the coiled coil. This second region appears to be conserved amongst widely divergent viral fusion-protein sequences. Its precise role in inhibitor action is unclear; while several escape mutations have been identified in this region, they are always restricted to the external HR-B helix of the coiled-coil. No mutations have been observed that alter the pocket within the internal HR-A helices where the HR-B residues found to mutate in escape mutants bind. Although not wishing to be bound by theory, it is hypothesised that mutations in this site are actually altering the inhibitor susceptibility of the RSV-F protein in the pre-fusogenic structure, rather than disrupting coiled-coil formation as is the currently accepted mode of action. According to the hypothesis, the HR-B residues shown to give rise to escape mutations must interact with parts of Site A in the pre-fusogenic structure. This is a simpler explanation of the widely separated sites of inhibitor binding identified by escape mutations than the only other possibility; that there are two separate sites (Site A and this region in the coiled-coil), both of which must be bound by inhibitor to cause inhibition.

Characterisation of Site A

The region of the RSV-F structure identified by this docking procedure is highlighted in FIG. 9. The majority of the protein is shown as a backbone worm, with one of the three symmetric Site A regions shown as a stick model. The topology of the site—a deep pocket beneath the top lip of the head region, above a groove that leads down to the adjacent radial channel—is dear. The site is also shown as a solvent excluded surface coloured by residue hydrophobicity (FIG. 9B), such that surfaces corresponding to hydrophobic residues are white, polar residues light grey and basic and acidic residues dark grey. This highlights the hydrophobic nature of the base of the Site A groove and the flanking charged residues that may form salt bridges across the groove. The residues that form Site A were specified by measuring the distance between docked ligands and the adjacent residues. All residues within 8 Å of any of the docked ligands were included in the definition of Site A.

TABLE 1
List of the PDB format atomic coordinates for one of the three Site A
regions in the RSV-F model.

| | | | | | | | | | | | |
|---|---|---|---|---|---|---|---|---|---|---|---|
| ATOM | 1 | N | TYR | A | 33 | −22.366 | 56.446 | −4.142 | 1.00 | 50.00 | N |
| ATOM | 2 | CA | TYR | A | 33 | −20.962 | 56.802 | −3.942 | 1.00 | 50.00 | C |
| ATOM | 3 | C | TYR | A | 33 | −19.843 | 56.187 | −4.779 | 1.00 | 50.00 | C |
| ATOM | 4 | O | TYR | A | 33 | −18.725 | 56.700 | −4.776 | 1.00 | 50.00 | O |
| ATOM | 5 | CB | TYR | A | 33 | −20.762 | 56.706 | −2.427 | 1.00 | 50.00 | C |
| ATOM | 6 | CG | TYR | A | 33 | −19.468 | 57.300 | −1.919 | 1.00 | 50.00 | C |
| ATOM | 7 | CD1 | TYR | A | 33 | −19.050 | 58.459 | −2.466 | 1.00 | 50.00 | C |
| ATOM | 8 | CD2 | TYR | A | 33 | −18.643 | 56.624 | −1.094 | 1.00 | 50.00 | C |
| ATOM | 9 | CE1 | TYR | A | 33 | −17.752 | 58.809 | −2.389 | 1.00 | 50.00 | C |
| ATOM | 10 | CE2 | TYR | A | 33 | −17.424 | 57.127 | −0.811 | 1.00 | 50.00 | C |
| ATOM | 11 | CZ | TYR | A | 33 | −16.941 | 58.159 | −1.533 | 1.00 | 50.00 | C |
| ATOM | 12 | OH | TYR | A | 33 | −15.696 | 58.695 | −1.327 | 1.00 | 50.00 | O |
| ATOM | 13 | N | CYS | A | 37 | −16.543 | 58.156 | −6.529 | 1.00 | 50.00 | N |
| ATOM | 14 | CA | CYS | A | 37 | −15.183 | 57.631 | −6.670 | 1.00 | 50.00 | C |
| ATOM | 15 | C | CYS | A | 37 | −14.567 | 56.857 | −5.510 | 1.00 | 50.00 | C |
| ATOM | 16 | O | CYS | A | 37 | −13.353 | 56.661 | −5.463 | 1.00 | 50.00 | O |
| ATOM | 17 | CB | CYS | A | 37 | −14.192 | 58.604 | −7.304 | 1.00 | 50.00 | C |
| ATOM | 18 | SG | CYS | A | 37 | −14.982 | 59.610 | −8.573 | 1.00 | 50.00 | S |
| ATOM | 19 | N | SER | A | 38 | −15.395 | 56.317 | −4.632 | 1.00 | 50.00 | N |
| ATOM | 20 | CA | SER | A | 38 | −15.007 | 55.240 | −3.723 | 1.00 | 50.00 | C |
| ATOM | 21 | C | SER | A | 38 | −15.810 | 53.972 | −3.971 | 1.00 | 50.00 | C |
| ATOM | 22 | O | SER | A | 38 | −17.011 | 53.917 | −4.233 | 1.00 | 50.00 | O |
| ATOM | 23 | CB | SER | A | 38 | −15.290 | 55.565 | −2.261 | 1.00 | 50.00 | C |
| ATOM | 24 | OG | SER | A | 38 | −14.390 | 56.532 | −1.743 | 1.00 | 50.00 | O |
| ATOM | 25 | N | ALA | A | 39 | −15.019 | 52.961 | −3.666 | 1.00 | 50.00 | N |
| ATOM | 26 | CA | ALA | A | 39 | −15.383 | 51.551 | −3.731 | 1.00 | 50.00 | C |
| ATOM | 27 | C | ALA | A | 39 | −15.577 | 51.048 | −2.308 | 1.00 | 50.00 | C |
| ATOM | 28 | O | ALA | A | 39 | −14.678 | 51.151 | −1.473 | 1.00 | 50.00 | O |
| ATOM | 29 | CB | ALA | A | 39 | −14.256 | 50.968 | −4.581 | 1.00 | 50.00 | C |
| ATOM | 30 | N | VAL | A | 40 | −16.739 | 50.476 | −2.038 | 1.00 | 50.00 | N |
| ATOM | 31 | CA | VAL | A | 40 | −16.959 | 49.650 | −0.850 | 1.00 | 50.00 | C |
| ATOM | 32 | C | VAL | A | 40 | −16.279 | 48.325 | −1.172 | 1.00 | 50.00 | C |
| ATOM | 33 | O | VAL | A | 40 | −16.786 | 47.519 | −1.952 | 1.00 | 50.00 | O |
| ATOM | 34 | CB | VAL | A | 40 | −18.431 | 49.521 | −0.447 | 1.00 | 50.00 | C |
| ATOM | 35 | CG1 | VAL | A | 40 | −18.731 | 48.435 | 0.586 | 1.00 | 50.00 | C |
| ATOM | 36 | CG2 | VAL | A | 40 | −19.013 | 50.832 | 0.081 | 1.00 | 50.00 | C |
| ATOM | 37 | N | SER | A | 41 | −15.135 | 48.109 | −0.543 | 1.00 | 50.00 | N |
| ATOM | 38 | CA | SER | A | 41 | −14.484 | 46.800 | −0.522 | 1.00 | 50.00 | C |
| ATOM | 39 | C | SER | A | 41 | −15.320 | 45.766 | 0.223 | 1.00 | 50.00 | C |
| ATOM | 40 | O | SER | A | 41 | −15.592 | 44.724 | −0.372 | 1.00 | 50.00 | O |
| ATOM | 41 | CB | SER | A | 41 | −13.061 | 46.905 | 0.025 | 1.00 | 50.00 | C |
| ATOM | 42 | OG | SER | A | 41 | −12.263 | 47.802 | −0.735 | 1.00 | 50.00 | O |
| ATOM | 43 | N | LYS | A | 42 | −15.715 | 46.008 | 1.464 | 1.00 | 50.00 | N |
| ATOM | 44 | CA | LYS | A | 42 | −16.327 | 44.992 | 2.321 | 1.00 | 50.00 | C |
| ATOM | 45 | C | LYS | A | 42 | −17.301 | 45.617 | 3.310 | 1.00 | 50.00 | C |
| ATOM | 46 | O | LYS | A | 42 | −17.044 | 46.671 | 3.891 | 1.00 | 50.00 | O |
| ATOM | 47 | CB | LYS | A | 42 | −15.272 | 44.162 | 3.055 | 1.00 | 50.00 | C |
| ATOM | 48 | CG | LYS | A | 42 | −14.479 | 43.268 | 2.103 | 1.00 | 50.00 | C |
| ATOM | 49 | CD | LYS | A | 42 | −13.490 | 42.291 | 2.732 | 1.00 | 50.00 | C |
| ATOM | 50 | CE | LYS | A | 42 | −12.762 | 41.546 | 1.615 | 1.00 | 50.00 | C |
| ATOM | 51 | NZ | LYS | A | 42 | −11.797 | 40.590 | 2.175 | 1.00 | 50.00 | N |
| ATOM | 52 | N | GLY | A | 43 | −18.402 | 44.922 | 3.540 | 1.00 | 50.00 | N |
| ATOM | 53 | CA | GLY | A | 43 | −19.300 | 45.185 | 4.663 | 1.00 | 50.00 | C |
| ATOM | 54 | C | GLY | A | 43 | −19.280 | 43.986 | 5.599 | 1.00 | 50.00 | C |
| ATOM | 55 | O | GLY | A | 43 | −19.303 | 42.820 | 5.207 | 1.00 | 50.00 | O |
| ATOM | 56 | N | LEU | A | 316 | −17.964 | 48.976 | 7.780 | 1.00 | 50.00 | N |
| ATOM | 57 | CA | LEU | B | 316 | −18.037 | 49.084 | 6.321 | 1.00 | 50.00 | C |
| ATOM | 58 | C | LEU | B | 316 | −16.727 | 49.672 | 5.807 | 1.00 | 50.00 | C |
| ATOM | 59 | O | LEU | B | 316 | −16.481 | 50.869 | 5.943 | 1.00 | 50.00 | O |
| ATOM | 60 | CB | LEU | B | 316 | −19.286 | 49.851 | 5.879 | 1.00 | 50.00 | C |
| ATOM | 61 | CG | LEU | B | 316 | −20.649 | 49.158 | 5.957 | 1.00 | 50.00 | C |
| ATOM | 62 | CD1 | LEU | B | 316 | −21.238 | 49.028 | 7.359 | 1.00 | 50.00 | C |
| ATOM | 63 | CD2 | LEU | B | 316 | −21.748 | 49.844 | 5.148 | 1.00 | 50.00 | C |
| ATOM | 64 | N | HIS | B | 317 | −15.893 | 48.849 | 5.190 | 1.00 | 50.00 | N |
| ATOM | 65 | CA | HIS | B | 317 | −14.608 | 49.224 | 4.598 | 1.00 | 50.00 | C |
| ATOM | 66 | C | HIS | B | 317 | −14.780 | 50.035 | 3.318 | 1.00 | 50.00 | C |
| ATOM | 67 | O | HIS | B | 317 | −15.501 | 49.645 | 2.400 | 1.00 | 50.00 | O |
| ATOM | 68 | CB | HIS | B | 317 | −13.748 | 47.982 | 4.354 | 1.00 | 50.00 | C |
| ATOM | 69 | CG | HIS | B | 317 | −12.276 | 48.210 | 4.085 | 1.00 | 50.00 | C |
| ATOM | 70 | ND1 | HIS | B | 317 | −11.290 | 48.042 | 5.039 | 1.00 | 50.00 | N |
| ATOM | 71 | CD2 | HIS | B | 317 | −11.714 | 48.615 | 2.877 | 1.00 | 50.00 | C |
| ATOM | 72 | CE1 | HIS | B | 317 | −10.184 | 48.386 | 4.291 | 1.00 | 50.00 | C |
| ATOM | 73 | NE2 | HIS | B | 317 | −10.339 | 48.700 | 2.958 | 1.00 | 50.00 | N |
| ATOM | 74 | N | THR | B | 318 | −14.064 | 51.146 | 3.271 | 1.00 | 50.00 | N |
| ATOM | 75 | CA | THR | B | 318 | −13.993 | 52.067 | 2.136 | 1.00 | 50.00 | C |
| ATOM | 76 | C | THR | B | 318 | −12.571 | 52.067 | 1.585 | 1.00 | 50.00 | C |
| ATOM | 77 | O | THR | B | 318 | −11.592 | 52.169 | 2.323 | 1.00 | 50.00 | O |
| ATOM | 78 | CB | THR | B | 318 | −14.447 | 53.443 | 2.627 | 1.00 | 50.00 | C |
| ATOM | 79 | OG1 | THR | B | 318 | −15.832 | 53.417 | 2.940 | 1.00 | 50.00 | O |
| ATOM | 80 | CG2 | THR | B | 318 | −14.235 | 54.620 | 1.676 | 1.00 | 50.00 | C |
| ATOM | 81 | N | SER | B | 319 | −12.474 | 52.049 | 0.265 | 1.00 | 50.00 | N |
| ATOM | 82 | CA | SER | B | 319 | −11.264 | 52.386 | −0.486 | 1.00 | 50.00 | C |
| ATOM | 83 | C | SER | B | 319 | −11.569 | 53.424 | −1.562 | 1.00 | 50.00 | C |
| ATOM | 84 | O | SER | B | 319 | −12.517 | 53.185 | −2.309 | 1.00 | 50.00 | O |
| ATOM | 85 | CB | SER | B | 319 | −10.666 | 51.153 | −1.161 | 1.00 | 50.00 | C |
| ATOM | 86 | OG | SER | B | 319 | −10.190 | 50.230 | −0.192 | 1.00 | 50.00 | O |
| ATOM | 87 | N | PRO | B | 320 | −10.829 | 54.505 | −1.768 | 1.00 | 50.00 | N |
| ATOM | 88 | CA | PRO | B | 320 | −11.053 | 55.437 | −2.873 | 1.00 | 50.00 | C |
| ATOM | 89 | C | PRO | B | 320 | −10.114 | 55.276 | −4.063 | 1.00 | 50.00 | C |
| ATOM | 90 | O | PRO | B | 320 | −8.892 | 55.320 | −3.923 | 1.00 | 50.00 | O |
| ATOM | 91 | CB | PRO | B | 320 | −10.939 | 56.796 | −2.186 | 1.00 | 50.00 | C |
| ATOM | 92 | CG | PRO | B | 320 | −9.904 | 56.585 | −1.084 | 1.00 | 50.00 | C |
| ATOM | 93 | CD | PRO | B | 320 | −9.944 | 55.093 | −0.763 | 1.00 | 50.00 | C |
| ATOM | 94 | N | LEU | B | 321 | −10.708 | 55.177 | −5.241 | 1.00 | 50.00 | N |
| ATOM | 95 | CA | LEU | B | 321 | −10.008 | 55.343 | −6.514 | 1.00 | 50.00 | C |
| ATOM | 96 | C | LEU | B | 321 | −9.840 | 56.814 | −6.870 | 1.00 | 50.00 | C |
| ATOM | 97 | O | LEU | B | 321 | −10.571 | 57.723 | −6.476 | 1.00 | 50.00 | O |
| ATOM | 98 | CB | LEU | B | 321 | −10.732 | 54.644 | −7.667 | 1.00 | 50.00 | C |
| ATOM | 99 | CG | LEU | B | 321 | −10.494 | 53.148 | −7.870 | 1.00 | 50.00 | C |
| ATOM | 100 | CD1 | LEU | B | 321 | −9.040 | 52.855 | −8.231 | 1.00 | 50.00 | C |
| ATOM | 101 | CD2 | LEU | B | 321 | −10.971 | 52.299 | −6.694 | 1.00 | 50.00 | C |
| ATOM | 102 | N | CYS | B | 322 | −8.862 | 56.966 | −7.744 | 1.00 | 50.00 | N |
| ATOM | 103 | CA | CYS | B | 322 | −8.587 | 58.200 | −8.474 | 1.00 | 50.00 | C |
| ATOM | 104 | C | CYS | B | 322 | −9.389 | 58.353 | −9.765 | 1.00 | 50.00 | C |
| ATOM | 105 | O | CYS | B | 322 | −8.928 | 57.973 | −10.840 | 1.00 | 50.00 | O |
| ATOM | 106 | CB | CYS | B | 322 | −7.061 | 58.117 | −8.497 | 1.00 | 50.00 | C |
| ATOM | 107 | SG | CYS | B | 322 | −6.438 | 58.685 | −6.902 | 1.00 | 50.00 | S |
| ATOM | 108 | N | THR | B | 323 | −10.530 | 59.022 | −9.676 | 1.00 | 50.00 | N |
| ATOM | 109 | CA | THR | B | 323 | −11.310 | 59.605 | −10.773 | 1.00 | 50.00 | C |
| ATOM | 110 | C | THR | B | 323 | −10.988 | 61.026 | −11.226 | 1.00 | 50.00 | C |
| ATOM | 111 | O | THR | B | 323 | −10.769 | 61.909 | −10.400 | 1.00 | 50.00 | O |
| ATOM | 112 | CB | THR | B | 323 | −12.758 | 59.654 | −10.280 | 1.00 | 50.00 | C |
| ATOM | 113 | OG1 | THR | B | 323 | −13.247 | 58.322 | −10.216 | 1.00 | 50.00 | O |
| ATOM | 114 | CG2 | THR | B | 323 | −13.805 | 60.491 | −11.020 | 1.00 | 50.00 | C |
| ATOM | 115 | N | SER | B | 330 | −2.630 | 64.514 | −11.521 | 1.00 | 50.00 | N |
| ATOM | 116 | CA | SER | B | 330 | −3.010 | 63.148 | −11.151 | 1.00 | 50.00 | C |
| ATOM | 117 | C | SER | B | 330 | −3.685 | 62.965 | −9.794 | 1.00 | 50.00 | C |
| ATOM | 118 | O | SER | B | 330 | −3.796 | 61.834 | −9.321 | 1.00 | 50.00 | O |
| ATOM | 119 | CB | SER | B | 330 | −1.797 | 62.223 | −11.246 | 1.00 | 50.00 | C |
| ATOM | 120 | OG | SER | B | 330 | −1.339 | 62.109 | −12.586 | 1.00 | 50.00 | O |
| ATOM | 121 | N | ASN | B | 331 | −4.188 | 64.017 | −9.168 | 1.00 | 50.00 | N |
| ATOM | 122 | CA | ASN | B | 331 | −4.797 | 63.972 | −7.840 | 1.00 | 50.00 | C |
| ATOM | 123 | C | ASN | B | 331 | −6.303 | 64.162 | −7.947 | 1.00 | 50.00 | C |
| ATOM | 124 | O | ASN | B | 331 | −6.803 | 65.127 | −8.524 | 1.00 | 50.00 | O |
| ATOM | 125 | CB | ASN | B | 331 | −4.139 | 64.960 | −6.878 | 1.00 | 50.00 | C |
| ATOM | 126 | CG | ASN | B | 331 | −4.300 | 66.473 | −6.888 | 1.00 | 50.00 | C |
| ATOM | 127 | OD1 | ASN | B | 331 | −3.317 | 67.211 | −6.927 | 1.00 | 50.00 | O |
| ATOM | 128 | ND2 | ASN | B | 331 | −5.514 | 66.987 | −6.812 | 1.00 | 50.00 | N |
| ATOM | 129 | N | ILE | B | 332 | −7.023 | 63.239 | −7.335 | 1.00 | 50.00 | N |
| ATOM | 130 | CA | ILE | B | 332 | −8.482 | 63.181 | −7.414 | 1.00 | 50.00 | C |
| ATOM | 131 | C | ILE | B | 332 | −8.732 | 63.706 | −6.011 | 1.00 | 50.00 | C |
| ATOM | 132 | O | ILE | B | 332 | −8.566 | 64.917 | −5.869 | 1.00 | 50.00 | O |
| ATOM | 133 | CB | ILE | B | 332 | −9.068 | 61.859 | −7.910 | 1.00 | 50.00 | C |
| ATOM | 134 | CG1 | ILE | B | 332 | −8.380 | 61.536 | −9.239 | 1.00 | 50.00 | C |
| ATOM | 135 | CG2 | ILE | B | 332 | −10.589 | 61.838 | −7.727 | 1.00 | 50.00 | C |
| ATOM | 136 | CD1 | ILE | B | 332 | −6.909 | 61.135 | −9.343 | 1.00 | 50.00 | C |
| ATOM | 137 | N | CYS | B | 333 | −9.057 | 63.041 | −4.916 | 1.00 | 50.00 | N |
| ATOM | 138 | CA | CYS | B | 333 | −9.218 | 61.605 | −4.684 | 1.00 | 50.00 | C |
| ATOM | 139 | C | CYS | B | 333 | −9.701 | 61.686 | −3.240 | 1.00 | 50.00 | C |
| ATOM | 140 | O | CYS | B | 333 | −9.074 | 62.264 | −2.352 | 1.00 | 50.00 | O |
| ATOM | 141 | CB | CYS | B | 333 | −8.024 | 60.654 | −4.748 | 1.00 | 50.00 | C |
| ATOM | 142 | SG | CYS | B | 333 | −6.952 | 60.491 | −6.191 | 1.00 | 50.00 | S |
| ATOM | 143 | N | LEU | B | 334 | −10.866 | 61.111 | −2.999 | 1.00 | 50.00 | N |
| ATOM | 144 | CA | LEU | B | 334 | −11.755 | 61.572 | −1.934 | 1.00 | 50.00 | C |
| ATOM | 145 | C | LEU | B | 334 | −11.265 | 61.320 | −0.515 | 1.00 | 50.00 | C |
| ATOM | 146 | O | LEU | B | 334 | −10.562 | 60.348 | −0.240 | 1.00 | 50.00 | O |
| ATOM | 147 | CB | LEU | B | 334 | −13.125 | 60.942 | −2.178 | 1.00 | 50.00 | C |
| ATOM | 148 | CG | LEU | B | 334 | −13.904 | 61.688 | −3.259 | 1.00 | 50.00 | C |
| ATOM | 149 | CD1 | LEU | B | 334 | −13.424 | 61.487 | −4.696 | 1.00 | 50.00 | C |
| ATOM | 150 | CD2 | LEU | B | 334 | −15.362 | 61.260 | −3.168 | 1.00 | 50.00 | C |
| ATOM | 151 | N | THR | B | 335 | −11.651 | 62.237 | 0.358 | 1.00 | 50.00 | N |
| ATOM | 152 | CA | THR | B | 335 | −11.246 | 62.219 | 1.763 | 1.00 | 50.00 | C |
| ATOM | 153 | C | THR | B | 335 | −12.381 | 61.570 | 2.543 | 1.00 | 50.00 | C |
| ATOM | 154 | O | THR | B | 335 | −13.383 | 62.199 | 2.882 | 1.00 | 50.00 | O |
| ATOM | 155 | CB | THR | B | 335 | −10.885 | 63.606 | 2.292 | 1.00 | 50.00 | C |
| ATOM | 156 | OG1 | THR | B | 335 | −9.780 | 64.112 | 1.557 | 1.00 | 50.00 | O |
| ATOM | 157 | CG2 | THR | B | 335 | −10.504 | 63.601 | 3.771 | 1.00 | 50.00 | C |
| ATOM | 158 | N | ARG | B | 336 | −12.154 | 60.299 | 2.826 | 1.00 | 50.00 | N |
| ATOM | 159 | CA | ARG | B | 336 | −12.990 | 59.499 | 3.717 | 1.00 | 50.00 | C |
| ATOM | 160 | C | ARG | B | 336 | −12.070 | 58.510 | 4.419 | 1.00 | 50.00 | C |
| ATOM | 161 | O | ARG | B | 336 | −10.905 | 58.283 | 4.093 | 1.00 | 50.00 | O |
| ATOM | 162 | CB | ARG | B | 336 | −14.131 | 58.789 | 2.988 | 1.00 | 50.00 | C |
| ATOM | 163 | CG | ARG | B | 336 | −15.044 | 59.766 | 2.253 | 1.00 | 50.00 | C |
| ATOM | 164 | CD | ARG | B | 336 | −16.297 | 59.101 | 1.698 | 1.00 | 50.00 | C |
| ATOM | 165 | NE | ARG | B | 336 | −17.039 | 60.147 | 0.996 | 1.00 | 50.00 | N |
| ATOM | 166 | CZ | ARG | B | 336 | −18.273 | 59.998 | 0.547 | 1.00 | 50.00 | C |
| ATOM | 167 | NH1 | ARG | B | 336 | −19.095 | 59.042 | 0.927 | 1.00 | 50.00 | N |
| ATOM | 168 | NH2 | ARG | B | 336 | −18.732 | 60.726 | −0.450 | 1.00 | 50.00 | N |
| ATOM | 169 | N | THR | B | 337 | −12.654 | 57.888 | 5.428 | 1.00 | 50.00 | N |
| ATOM | 170 | CA | THR | B | 337 | −11.947 | 56.932 | 6.277 | 1.00 | 50.00 | C |
| ATOM | 171 | C | THR | B | 337 | −11.813 | 55.554 | 5.640 | 1.00 | 50.00 | C |
| ATOM | 172 | O | THR | B | 337 | −12.525 | 55.204 | 4.699 | 1.00 | 50.00 | O |
| ATOM | 173 | CB | THR | B | 337 | −12.646 | 56.938 | 7.635 | 1.00 | 50.00 | C |
| ATOM | 174 | OG1 | THR | B | 337 | −13.984 | 56.462 | 7.594 | 1.00 | 50.00 | O |
| ATOM | 175 | CG2 | THR | B | 337 | −12.550 | 58.254 | 8.405 | 1.00 | 50.00 | C |
| ATOM | 176 | N | ASP | B | 338 | −10.888 | 54.768 | 6.164 | 1.00 | 50.00 | N |
| ATOM | 177 | CA | ASP | B | 338 | −10.687 | 53.374 | 5.763 | 1.00 | 50.00 | C |
| ATOM | 178 | C | ASP | B | 338 | −11.796 | 52.405 | 6.157 | 1.00 | 50.00 | C |
| ATOM | 179 | O | ASP | B | 338 | −12.080 | 51.501 | 5.372 | 1.00 | 50.00 | O |
| ATOM | 180 | CB | ASP | B | 338 | −9.331 | 52.869 | 6.257 | 1.00 | 50.00 | C |
| ATOM | 181 | CG | ASP | B | 338 | −8.108 | 53.522 | 5.628 | 1.00 | 50.00 | C |
| ATOM | 182 | OD1 | ASP | B | 338 | −7.956 | 53.458 | 4.389 | 1.00 | 50.00 | O |
| ATOM | 183 | OD2 | ASP | B | 338 | −7.289 | 54.109 | 6.367 | 1.00 | 50.00 | O |
| ATOM | 184 | N | ARG | B | 339 | −12.385 | 52.531 | 7.335 | 1.00 | 50.00 | N |
| ATOM | 185 | CA | ARG | B | 339 | −13.447 | 51.661 | 7.842 | 1.00 | 50.00 | C |
| ATOM | 186 | C | ARG | B | 339 | −14.473 | 52.543 | 8.546 | 1.00 | 50.00 | C |
| ATOM | 187 | O | ARG | B | 339 | −14.081 | 53.466 | 9.258 | 1.00 | 50.00 | O |
| ATOM | 188 | CB | ARG | B | 339 | −12.881 | 50.560 | 8.741 | 1.00 | 50.00 | C |
| ATOM | 189 | CG | ARG | B | 339 | −11.853 | 49.648 | 8.072 | 1.00 | 50.00 | C |
| ATOM | 190 | CD | ARG | B | 339 | −11.315 | 48.476 | 8.890 | 1.00 | 50.00 | C |
| ATOM | 191 | NE | ARG | B | 339 | −10.193 | 47.853 | 8.186 | 1.00 | 50.00 | N |
| ATOM | 192 | CZ | ARG | B | 339 | −9.443 | 46.869 | 8.654 | 1.00 | 50.00 | C |
| ATOM | 193 | NH1 | ARG | B | 339 | −9.652 | 46.264 | 9.809 | 1.00 | 50.00 | N |
| ATOM | 194 | NH2 | ARG | B | 339 | −8.420 | 46.463 | 7.926 | 1.00 | 50.00 | N |
| ATOM | 195 | N | PHE | B | 352 | −15.976 | 56.606 | 12.767 | 1.00 | 50.00 | N |
| ATOM | 196 | CA | PHE | B | 352 | −14.758 | 55.914 | 12.339 | 1.00 | 50.00 | C |
| ATOM | 197 | C | PHE | B | 352 | −14.373 | 54.842 | 13.353 | 1.00 | 50.00 | C |
| ATOM | 198 | O | PHE | B | 352 | −13.743 | 55.222 | 14.338 | 1.00 | 50.00 | O |
| ATOM | 199 | CB | PHE | B | 352 | −13.515 | 56.768 | 12.070 | 1.00 | 50.00 | C |
| ATOM | 200 | CG | PHE | B | 352 | −12.223 | 56.071 | 11.696 | 1.00 | 50.00 | C |
| ATOM | 201 | CD1 | PHE | B | 352 | −12.211 | 55.388 | 10.534 | 1.00 | 50.00 | C |
| ATOM | 202 | CD2 | PHE | B | 352 | −11.115 | 56.066 | 12.467 | 1.00 | 50.00 | C |
| ATOM | 203 | CE1 | PHE | B | 352 | −11.107 | 54.734 | 10.120 | 1.00 | 50.00 | C |
| ATOM | 204 | CE2 | PHE | B | 352 | −10.006 | 55.416 | 12.061 | 1.00 | 50.00 | C |
| ATOM | 205 | CZ | PHE | B | 352 | −9.999 | 54.755 | 10.886 | 1.00 | 50.00 | C |
| ATOM | 206 | N | LYS | B | 394 | −15.007 | 65.977 | −0.301 | 1.00 | 50.00 | N |
| ATOM | 207 | CA | LYS | B | 394 | −13.816 | 66.819 | −0.356 | 1.00 | 50.00 | C |
| ATOM | 208 | C | LYS | B | 394 | −12.689 | 65.940 | −0.874 | 1.00 | 50.00 | C |
| ATOM | 209 | O | LYS | B | 394 | −11.930 | 65.260 | −0.185 | 1.00 | 50.00 | O |
| ATOM | 210 | CB | LYS | B | 394 | −13.517 | 67.496 | 0.978 | 1.00 | 50.00 | C |
| ATOM | 211 | CG | LYS | B | 394 | −14.630 | 68.490 | 1.302 | 1.00 | 50.00 | C |
| ATOM | 212 | CD | LYS | B | 394 | −14.238 | 69.349 | 2.499 | 1.00 | 50.00 | C |
| ATOM | 213 | CE | LYS | B | 394 | −15.393 | 70.297 | 2.805 | 1.00 | 50.00 | C |
| ATOM | 214 | NZ | LYS | B | 394 | −15.079 | 71.028 | 4.041 | 1.00 | 50.00 | N |
| ATOM | 215 | N | ILE | B | 395 | −12.642 | 66.080 | −2.185 | 1.00 | 50.00 | N |
| ATOM | 216 | CA | ILE | B | 395 | −11.576 | 65.644 | −3.082 | 1.00 | 50.00 | C |
| ATOM | 217 | C | ILE | B | 395 | −10.278 | 66.376 | −2.746 | 1.00 | 50.00 | C |
| ATOM | 218 | O | ILE | B | 395 | −10.299 | 67.574 | −2.467 | 1.00 | 50.00 | O |
| ATOM | 219 | CB | ILE | B | 395 | −12.245 | 65.901 | −4.438 | 1.00 | 50.00 | C |
| ATOM | 220 | CG1 | ILE | B | 395 | −13.435 | 65.003 | −4.790 | 1.00 | 50.00 | C |
| ATOM | 221 | CG2 | ILE | B | 395 | −11.196 | 65.720 | −5.529 | 1.00 | 50.00 | C |
| ATOM | 222 | CD1 | ILE | B | 395 | −13.851 | 64.752 | −6.239 | 1.00 | 50.00 | C |
| ATOM | 223 | N | MET | B | 396 | −9.150 | 65.685 | −2.789 | 1.00 | 50.00 | N |
| ATOM | 224 | CA | MET | B | 396 | −7.821 | 66.285 | −2.654 | 1.00 | 50.00 | C |
| ATOM | 225 | C | MET | B | 396 | −6.735 | 65.579 | −3.456 | 1.00 | 50.00 | C |
| ATOM | 226 | O | MET | B | 396 | −6.062 | 66.244 | −4.241 | 1.00 | 50.00 | O |
| ATOM | 227 | CB | MET | B | 396 | −7.316 | 66.435 | −1.217 | 1.00 | 50.00 | C |
| ATOM | 228 | CG | MET | B | 396 | −7.702 | 67.763 | −0.573 | 1.00 | 50.00 | C |
| ATOM | 229 | SD | MET | B | 396 | −7.042 | 67.843 | 1.099 | 1.00 | 50.00 | S |
| ATOM | 230 | CE | MET | B | 396 | −7.813 | 69.411 | 1.531 | 1.00 | 50.00 | C |
| ATOM | 231 | N | THR | B | 397 | −6.456 | 64.319 | −3.173 | 1.00 | 50.00 | N |
| ATOM | 232 | CA | THR | B | 397 | −5.330 | 63.556 | −3.711 | 1.00 | 50.00 | C |
| ATOM | 233 | C | THR | B | 397 | −5.433 | 62.207 | −3.018 | 1.00 | 50.00 | C |
| ATOM | 234 | O | THR | B | 397 | −5.714 | 62.096 | −1.824 | 1.00 | 50.00 | O |
| ATOM | 235 | CB | THR | B | 397 | −3.905 | 64.103 | −3.595 | 1.00 | 50.00 | C |
| ATOM | 236 | OG1 | THR | B | 397 | −3.017 | 63.291 | −4.351 | 1.00 | 50.00 | O |
| ATOM | 237 | CG2 | THR | B | 397 | −3.310 | 64.273 | −2.199 | 1.00 | 50.00 | C |
| ATOM | 238 | N | SER | B | 398 | −5.115 | 61.171 | −3.777 | 1.00 | 50.00 | N |
| ATOM | 239 | CA | SER | B | 398 | −4.997 | 59.839 | −3.189 | 1.00 | 50.00 | C |
| ATOM | 240 | C | SER | B | 398 | −3.678 | 59.865 | −2.431 | 1.00 | 50.00 | C |
| ATOM | 241 | O | SER | B | 398 | −2.796 | 60.708 | −2.596 | 1.00 | 50.00 | O |
| ATOM | 242 | CB | SER | B | 398 | −5.145 | 58.706 | −4.203 | 1.00 | 50.00 | C |
| ATOM | 243 | OG | SER | B | 398 | −4.192 | 58.795 | −5.253 | 1.00 | 50.00 | O |
| ATOM | 244 | N | LYS | B | 399 | −3.541 | 58.871 | −1.575 | 1.00 | 50.00 | N |
| ATOM | 245 | CA | LYS | B | 399 | −2.256 | 58.632 | −0.918 | 1.00 | 50.00 | C |
| ATOM | 246 | C | LYS | B | 399 | −1.177 | 58.107 | −1.862 | 1.00 | 50.00 | C |
| ATOM | 247 | O | LYS | B | 399 | −0.361 | 57.317 | −1.393 | 1.00 | 50.00 | O |
| ATOM | 248 | CB | LYS | B | 399 | −2.644 | 57.735 | 0.259 | 1.00 | 50.00 | C |
| ATOM | 249 | CG | LYS | B | 399 | −3.376 | 58.510 | 1.353 | 1.00 | 50.00 | C |
| ATOM | 250 | CD | LYS | B | 399 | −3.720 | 57.676 | 2.584 | 1.00 | 50.00 | C |
| ATOM | 251 | CE | LYS | B | 399 | −4.451 | 58.580 | 3.573 | 1.00 | 50.00 | C |
| ATOM | 252 | NZ | LYS | B | 399 | −4.692 | 57.868 | 4.836 | 1.00 | 50.00 | N |
| ATOM | 253 | N | THR | B | 400 | −1.249 | 58.273 | −3.177 | 1.00 | 50.00 | N |
| ATOM | 254 | CA | THR | B | 400 | −1.078 | 57.152 | −4.103 | 1.00 | 50.00 | C |
| ATOM | 255 | C | THR | B | 400 | −0.579 | 57.562 | −5.486 | 1.00 | 50.00 | C |
| ATOM | 256 | O | THR | B | 400 | −1.305 | 58.281 | −6.172 | 1.00 | 50.00 | O |
| ATOM | 257 | CB | THR | B | 400 | −2.159 | 56.070 | −4.045 | 1.00 | 50.00 | C |
| ATOM | 258 | OG1 | THR | B | 400 | −3.403 | 56.427 | −4.625 | 1.00 | 50.00 | O |
| ATOM | 259 | CG2 | THR | B | 400 | −2.490 | 55.577 | −2.637 | 1.00 | 50.00 | C |
| ATOM | 260 | N | ASP | B | 401 | 0.608 | 57.159 | −5.917 | 1.00 | 50.00 | N |
| ATOM | 261 | CA | ASP | B | 401 | 1.266 | 57.673 | −7.122 | 1.00 | 50.00 | C |
| ATOM | 262 | C | ASP | B | 401 | 0.548 | 57.226 | −8.390 | 1.00 | 50.00 | C |
| ATOM | 263 | O | ASP | B | 401 | 0.079 | 58.079 | −9.142 | 1.00 | 50.00 | O |
| ATOM | 264 | CB | ASP | B | 401 | 2.774 | 57.439 | −7.246 | 1.00 | 50.00 | C |
| ATOM | 265 | CG | ASP | B | 401 | 3.473 | 58.253 | −8.326 | 1.00 | 50.00 | C |
| ATOM | 266 | OD1 | ASP | B | 401 | 3.593 | 59.487 | −8.164 | 1.00 | 50.00 | O |
| ATOM | 267 | OD2 | ASP | B | 401 | 3.904 | 57.677 | −9.348 | 1.00 | 50.00 | O |
| ATOM | 268 | N | VAL | B | 402 | 0.507 | 55.926 | −8.631 | 1.00 | 50.00 | N |
| ATOM | 269 | CA | VAL | B | 402 | −0.129 | 55.327 | −9.803 | 1.00 | 50.00 | C |
| ATOM | 270 | C | VAL | B | 402 | −0.668 | 53.997 | −9.294 | 1.00 | 50.00 | C |
| ATOM | 271 | O | VAL | B | 402 | 0.049 | 53.173 | −8.727 | 1.00 | 50.00 | O |
| ATOM | 272 | CB | VAL | B | 402 | 0.797 | 55.126 | −11.006 | 1.00 | 50.00 | C |
| ATOM | 273 | CG1 | VAL | B | 402 | 0.179 | 54.320 | −12.149 | 1.00 | 50.00 | C |
| ATOM | 274 | CG2 | VAL | B | 402 | 1.298 | 56.444 | −11.595 | 1.00 | 50.00 | C |
| ATOM | 275 | N | SER | B | 403 | −1.929 | 53.806 | −9.637 | 1.00 | 50.00 | N |
| ATOM | 276 | CA | SER | B | 403 | −2.673 | 52.548 | −9.614 | 1.00 | 50.00 | C |
| ATOM | 277 | C | SER | B | 403 | −4.094 | 53.045 | −9.849 | 1.00 | 50.00 | C |
| ATOM | 278 | O | SER | B | 403 | −4.880 | 53.032 | −8.902 | 1.00 | 50.00 | O |
| ATOM | 279 | CB | SER | B | 403 | −2.547 | 51.738 | −8.322 | 1.00 | 50.00 | C |
| ATOM | 280 | OG | SER | B | 403 | −3.082 | 50.430 | −8.460 | 1.00 | 50.00 | O |
| ATOM | 281 | N | SER | B | 404 | −4.467 | 53.530 | −11.025 | 1.00 | 50.00 | N |
| ATOM | 282 | CA | SER | B | 404 | −5.812 | 54.065 | −11.247 | 1.00 | 50.00 | C |
| ATOM | 283 | C | SER | B | 404 | −6.769 | 52.935 | −11.609 | 1.00 | 50.00 | C |
| ATOM | 284 | O | SER | B | 404 | −7.211 | 52.776 | −12.746 | 1.00 | 50.00 | O |
| ATOM | 285 | CB | SER | B | 404 | −5.782 | 55.208 | −12.260 | 1.00 | 50.00 | C |
| ATOM | 286 | OG | SER | B | 404 | −5.081 | 56.339 | −11.763 | 1.00 | 50.00 | O |
| ATOM | 287 | N | SER | B | 405 | −7.007 | 52.101 | −10.610 | 1.00 | 50.00 | N |
| ATOM | 288 | CA | SER | B | 405 | −7.066 | 50.643 | −10.707 | 1.00 | 50.00 | C |
| ATOM | 289 | C | SER | B | 405 | −6.690 | 50.019 | −9.368 | 1.00 | 50.00 | C |
| ATOM | 290 | O | SER | B | 405 | −5.666 | 50.339 | −8.768 | 1.00 | 50.00 | O |
| ATOM | 291 | CB | SER | B | 405 | −6.401 | 49.926 | −11.882 | 1.00 | 50.00 | C |
| ATOM | 292 | OG | SER | B | 405 | −4.997 | 49.775 | −11.744 | 1.00 | 50.00 | O |
| ATOM | 293 | N | VAL | B | 406 | −7.530 | 49.118 | −8.889 | 1.00 | 50.00 | N |
| ATOM | 294 | CA | VAL | B | 406 | −7.406 | 48.428 | −7.604 | 1.00 | 50.00 | C |
| ATOM | 295 | C | VAL | B | 406 | −8.025 | 47.054 | −7.829 | 1.00 | 50.00 | C |
| ATOM | 296 | O | VAL | B | 406 | −9.044 | 46.907 | −8.504 | 1.00 | 50.00 | O |
| ATOM | 297 | CB | VAL | B | 406 | −8.104 | 49.167 | −6.457 | 1.00 | 50.00 | C |
| ATOM | 298 | CG1 | VAL | B | 406 | −8.290 | 48.379 | −5.160 | 1.00 | 50.00 | C |
| ATOM | 299 | CG2 | VAL | B | 406 | −7.404 | 50.473 | −6.084 | 1.00 | 50.00 | C |
| ATOM | 300 | N | ILE | B | 407 | −7.494 | 46.067 | −7.127 | 1.00 | 50.00 | N |
| ATOM | 301 | CA | ILE | B | 407 | −8.162 | 44.778 | −6.950 | 1.00 | 50.00 | C |
| ATOM | 302 | C | ILE | B | 407 | −9.156 | 44.711 | −5.795 | 1.00 | 50.00 | C |
| ATOM | 303 | O | ILE | B | 407 | −8.941 | 45.277 | −4.724 | 1.00 | 50.00 | O |
| ATOM | 304 | CB | ILE | B | 407 | −7.119 | 43.658 | −6.964 | 1.00 | 50.00 | C |
| ATOM | 305 | CG1 | ILE | B | 407 | −7.670 | 42.461 | −7.735 | 1.00 | 50.00 | C |
| ATOM | 306 | CG2 | ILE | B | 407 | −6.615 | 43.261 | −5.578 | 1.00 | 50.00 | C |
| ATOM | 307 | CD1 | ILE | B | 407 | −7.685 | 42.714 | −9.242 | 1.00 | 50.00 | C |
| ATOM | 308 | N | THR | B | 408 | −10.187 | 43.906 | −5.986 | 1.00 | 50.00 | N |
| ATOM | 309 | CA | THR | B | 408 | −11.226 | 43.617 | −4.997 | 1.00 | 50.00 | C |
| ATOM | 310 | C | THR | B | 408 | −11.881 | 42.312 | −5.431 | 1.00 | 50.00 | C |
| ATOM | 311 | O | THR | B | 408 | −12.440 | 42.270 | −6.527 | 1.00 | 50.00 | O |
| ATOM | 312 | CB | THR | B | 408 | −12.267 | 44.737 | −4.957 | 1.00 | 50.00 | C |
| ATOM | 313 | OG1 | THR | B | 408 | −11.682 | 45.918 | −4.428 | 1.00 | 50.00 | O |
| ATOM | 314 | CG2 | THR | B | 408 | −13.540 | 44.453 | −4.162 | 1.00 | 50.00 | C |
| ATOM | 315 | N | SER | B | 409 | −11.927 | 41.331 | −4.543 | 1.00 | 50.00 | N |
| ATOM | 316 | CA | SER | B | 409 | −12.766 | 40.135 | −4.649 | 1.00 | 50.00 | C |
| ATOM | 317 | C | SER | B | 409 | −12.836 | 39.543 | −6.050 | 1.00 | 50.00 | C |
| ATOM | 318 | O | SER | B | 409 | −13.831 | 39.767 | −6.739 | 1.00 | 50.00 | O |
| ATOM | 319 | CB | SER | B | 409 | −14.190 | 40.364 | −4.140 | 1.00 | 50.00 | C |
| ATOM | 320 | OG | SER | B | 409 | −14.246 | 40.663 | −2.753 | 1.00 | 50.00 | O |
| ATOM | 321 | N | LEU | B | 410 | −11.771 | 38.935 | −6.544 | 1.00 | 50.00 | N |
| ATOM | 322 | CA | LEU | B | 410 | −11.731 | 38.460 | −7.927 | 1.00 | 50.00 | C |
| ATOM | 323 | C | LEU | B | 410 | −11.809 | 39.485 | −9.056 | 1.00 | 50.00 | C |
| ATOM | 324 | O | LEU | B | 410 | −12.321 | 39.182 | −10.132 | 1.00 | 50.00 | O |
| ATOM | 325 | CB | LEU | B | 410 | −12.605 | 37.211 | −8.060 | 1.00 | 50.00 | C |
| ATOM | 326 | CG | LEU | B | 410 | −14.127 | 37.198 | −8.214 | 1.00 | 50.00 | C |
| ATOM | 327 | CD1 | LEU | B | 410 | −14.823 | 38.221 | −9.113 | 1.00 | 50.00 | C |
| ATOM | 328 | CD2 | LEU | B | 410 | −14.508 | 35.826 | −8.765 | 1.00 | 50.00 | C |
| ATOM | 329 | N | GLY | B | 411 | −11.380 | 40.730 | −8.934 | 1.00 | 50.00 | N |
| ATOM | 330 | CA | GLY | B | 411 | −11.866 | 41.764 | −9.847 | 1.00 | 50.00 | C |
| ATOM | 331 | C | GLY | B | 411 | −11.120 | 43.086 | −9.794 | 1.00 | 50.00 | C |
| ATOM | 332 | O | GLY | B | 411 | −10.611 | 43.456 | −8.738 | 1.00 | 50.00 | O |
| ATOM | 333 | N | ALA | B | 412 | −11.086 | 43.794 | −10.910 | 1.00 | 50.00 | N |
| ATOM | 334 | CA | ALA | B | 412 | −10.262 | 44.994 | −11.045 | 1.00 | 50.00 | C |
| ATOM | 335 | C | ALA | B | 412 | −11.148 | 46.208 | −11.291 | 1.00 | 50.00 | C |
| ATOM | 336 | O | ALA | B | 412 | −11.487 | 46.491 | −12.438 | 1.00 | 50.00 | O |
| ATOM | 337 | CB | ALA | B | 412 | −9.214 | 44.743 | −12.129 | 1.00 | 50.00 | C |
| ATOM | 338 | N | ILE | B | 413 | −11.489 | 46.951 | −10.250 | 1.00 | 50.00 | N |
| ATOM | 339 | CA | ILE | B | 413 | −12.125 | 48.267 | −10.344 | 1.00 | 50.00 | C |
| ATOM | 340 | C | ILE | B | 413 | −11.091 | 49.268 | −10.844 | 1.00 | 50.00 | C |
| ATOM | 341 | O | ILE | B | 413 | −10.055 | 49.407 | −10.196 | 1.00 | 50.00 | O |
| ATOM | 342 | CB | ILE | B | 413 | −12.724 | 48.673 | −8.994 | 1.00 | 50.00 | C |
| ATOM | 343 | CG1 | ILE | B | 413 | −14.007 | 47.900 | −8.679 | 1.00 | 50.00 | C |
| ATOM | 344 | CG2 | ILE | B | 413 | −12.985 | 50.164 | −8.780 | 1.00 | 50.00 | C |
| ATOM | 345 | CD1 | ILE | B | 413 | −14.006 | 47.271 | −7.288 | 1.00 | 50.00 | C |
| ATOM | 346 | N | VAL | B | 414 | −11.363 | 49.954 | −11.941 | 1.00 | 50.00 | N |
| ATOM | 347 | CA | VAL | B | 414 | −10.365 | 50.767 | −12.637 | 1.00 | 50.00 | C |
| ATOM | 348 | C | VAL | B | 414 | −10.991 | 52.078 | −13.103 | 1.00 | 50.00 | C |
| ATOM | 349 | O | VAL | B | 414 | −12.097 | 52.068 | −13.644 | 1.00 | 50.00 | O |
| ATOM | 350 | CB | VAL | B | 414 | −9.569 | 49.978 | −13.681 | 1.00 | 50.00 | C |
| ATOM | 351 | CG1 | VAL | B | 414 | −9.171 | 48.530 | −13.394 | 1.00 | 50.00 | C |
| ATOM | 352 | CG2 | VAL | B | 414 | −10.223 | 49.913 | −15.059 | 1.00 | 50.00 | C |
| ATOM | 353 | N | SER | B | 415 | −10.277 | 53.188 | −13.004 | 1.00 | 50.00 | N |
| ATOM | 354 | CA | SER | B | 415 | −10.632 | 54.437 | −13.679 | 1.00 | 50.00 | C |
| ATOM | 355 | C | SER | B | 415 | −10.236 | 54.371 | −15.150 | 1.00 | 50.00 | C |
| ATOM | 356 | O | SER | B | 415 | −9.154 | 54.810 | −15.540 | 1.00 | 50.00 | O |
| ATOM | 357 | CB | SER | B | 415 | −9.996 | 55.634 | −12.971 | 1.00 | 50.00 | C |
| ATOM | 358 | OG | SER | B | 415 | −10.441 | 55.767 | −11.629 | 1.00 | 50.00 | O |
| ATOM | 359 | N | LYS | B | 419 | −7.797 | 57.692 | −16.076 | 1.00 | 50.00 | N |
| ATOM | 360 | CA | LYS | B | 419 | −6.438 | 57.414 | −16.541 | 1.00 | 50.00 | C |
| ATOM | 361 | C | LYS | B | 419 | −6.247 | 57.337 | −18.053 | 1.00 | 50.00 | C |
| ATOM | 362 | O | LYS | B | 419 | −5.372 | 58.062 | −18.523 | 1.00 | 50.00 | O |
| ATOM | 363 | CB | LYS | B | 419 | −5.923 | 56.142 | −15.867 | 1.00 | 50.00 | C |
| ATOM | 364 | CG | LYS | B | 419 | −4.423 | 55.885 | −16.008 | 1.00 | 50.00 | C |
| ATOM | 365 | CD | LYS | B | 419 | −3.611 | 56.622 | −14.945 | 1.00 | 50.00 | C |
| ATOM | 366 | CE | LYS | B | 419 | −2.097 | 56.476 | −15.069 | 1.00 | 50.00 | C |
| ATOM | 367 | NZ | LYS | B | 419 | −1.473 | 57.143 | −13.917 | 1.00 | 50.00 | N |
| ATOM | 368 | N | LYS | B | 421 | −6.707 | 53.630 | −18.981 | 1.00 | 50.00 | N |
| ATOM | 369 | CA | LYS | B | 421 | −6.352 | 52.218 | −18.826 | 1.00 | 50.00 | C |
| ATOM | 370 | C | LYS | B | 421 | −6.604 | 51.212 | −19.945 | 1.00 | 50.00 | C |
| ATOM | 371 | O | LYS | B | 421 | −5.649 | 50.623 | −20.449 | 1.00 | 50.00 | O |
| ATOM | 372 | CB | LYS | B | 421 | −6.902 | 51.714 | −17.489 | 1.00 | 50.00 | C |
| ATOM | 373 | CG | LYS | B | 421 | −6.289 | 52.213 | −16.181 | 1.00 | 50.00 | C |
| ATOM | 374 | CD | LYS | B | 421 | −4.904 | 51.658 | −15.861 | 1.00 | 50.00 | C |
| ATOM | 375 | CE | LYS | B | 421 | −4.339 | 52.235 | −14.564 | 1.00 | 50.00 | C |
| ATOM | 376 | NZ | LYS | B | 421 | −2.892 | 51.996 | −14.463 | 1.00 | 50.00 | N |
| ATOM | 377 | N | ASP | B | 440 | −15.743 | 55.173 | −12.585 | 1.00 | 50.00 | N |
| ATOM | 378 | CA | ASP | B | 440 | −14.930 | 53.991 | −12.305 | 1.00 | 50.00 | C |
| ATOM | 379 | C | ASP | B | 440 | −15.709 | 52.816 | −12.883 | 1.00 | 50.00 | C |
| ATOM | 380 | O | ASP | B | 440 | −16.880 | 52.592 | −12.578 | 1.00 | 50.00 | O |
| ATOM | 381 | CB | ASP | B | 440 | −14.734 | 53.742 | −10.809 | 1.00 | 50.00 | C |
| ATOM | 382 | CG | ASP | B | 440 | −13.776 | 54.672 | −10.080 | 1.00 | 50.00 | C |
| ATOM | 383 | OD1 | ASP | B | 440 | −12.589 | 54.731 | −10.468 | 1.00 | 50.00 | O |
| ATOM | 384 | OD2 | ASP | B | 440 | −14.187 | 55.334 | −9.103 | 1.00 | 50.00 | O |
| ATOM | 385 | N | LEU | E | 48 | 0.984 | 43.058 | −9.181 | 1.00 | 50.00 | N |
| ATOM | 386 | CA | LEU | E | 48 | −0.436 | 43.190 | −8.851 | 1.00 | 50.00 | C |
| ATOM | 387 | C | LEU | E | 48 | −1.158 | 41.865 | −9.057 | 1.00 | 50.00 | C |
| ATOM | 388 | O | LEU | E | 48 | −0.856 | 41.101 | −9.973 | 1.00 | 50.00 | O |
| ATOM | 389 | CB | LEU | E | 48 | −1.146 | 44.305 | −9.622 | 1.00 | 50.00 | C |
| ATOM | 390 | CG | LEU | E | 48 | −0.834 | 45.752 | −9.235 | 1.00 | 50.00 | C |
| ATOM | 391 | CD1 | LEU | E | 48 | 0.655 | 46.082 | −9.171 | 1.00 | 50.00 | C |
| ATOM | 392 | CD2 | LEU | E | 48 | −1.488 | 46.811 | −10.120 | 1.00 | 50.00 | C |
| ATOM | 393 | N | ARG | E | 49 | −2.113 | 41.588 | −8.184 | 1.00 | 50.00 | N |
| ATOM | 394 | CA | ARG | E | 49 | −2.693 | 40.253 | −8.036 | 1.00 | 50.00 | C |
| ATOM | 395 | C | ARG | E | 49 | −4.172 | 40.303 | −7.694 | 1.00 | 50.00 | C |
| ATOM | 396 | O | ARG | E | 49 | −4.716 | 41.333 | −7.299 | 1.00 | 50.00 | O |
| ATOM | 397 | CB | ARG | E | 49 | −2.066 | 39.442 | −6.903 | 1.00 | 50.00 | C |
| ATOM | 398 | CG | ARG | E | 49 | −0.568 | 39.286 | −7.129 | 1.00 | 50.00 | C |
| ATOM | 399 | CD | ARG | E | 49 | −0.020 | 38.123 | −6.315 | 1.00 | 50.00 | C |
| ATOM | 400 | NE | ARG | E | 49 | 1.427 | 38.322 | −6.318 | 1.00 | 50.00 | N |
| ATOM | 401 | CZ | ARG | E | 49 | 2.324 | 37.544 | −5.744 | 1.00 | 50.00 | C |
| ATOM | 402 | NH1 | ARG | E | 49 | 1.915 | 36.441 | −5.157 | 1.00 | 50.00 | N |
| ATOM | 403 | NH2 | ARG | E | 49 | 3.601 | 37.869 | −5.677 | 1.00 | 50.00 | N |
| ATOM | 404 | N | THR | E | 50 | −4.772 | 39.130 | −7.813 | 1.00 | 50.00 | N |
| ATOM | 405 | CA | THR | E | 50 | −6.188 | 38.931 | −7.521 | 1.00 | 50.00 | C |
| ATOM | 406 | C | THR | E | 50 | −6.406 | 37.460 | −7.195 | 1.00 | 50.00 | C |
| ATOM | 407 | O | THR | E | 50 | −6.041 | 36.618 | −8.014 | 1.00 | 50.00 | O |
| ATOM | 408 | CB | THR | E | 50 | −6.940 | 39.240 | −8.814 | 1.00 | 50.00 | C |
| ATOM | 409 | OG1 | THR | E | 50 | −6.445 | 40.357 | −9.538 | 1.00 | 50.00 | O |
| ATOM | 410 | CG2 | THR | E | 50 | −8.449 | 39.345 | −8.632 | 1.00 | 50.00 | C |
| ATOM | 411 | N | LYS | F | 315 | 10.770 | 47.943 | −5.438 | 1.00 | 50.00 | N |
| ATOM | 412 | CA | LYS | F | 315 | 10.670 | 48.524 | −4.095 | 1.00 | 50.00 | C |
| ATOM | 413 | C | LYS | F | 315 | 11.916 | 48.320 | −3.240 | 1.00 | 50.00 | C |
| ATOM | 414 | O | LYS | F | 315 | 12.419 | 47.200 | −3.158 | 1.00 | 50.00 | O |
| ATOM | 415 | CB | LYS | F | 315 | 9.545 | 47.906 | −3.262 | 1.00 | 50.00 | C |
| ATOM | 416 | CG | LYS | F | 315 | 8.139 | 48.248 | −3.746 | 1.00 | 50.00 | C |
| ATOM | 417 | CD | LYS | F | 315 | 6.981 | 47.656 | −2.946 | 1.00 | 50.00 | C |
| ATOM | 418 | CE | LYS | F | 315 | 5.658 | 48.058 | −3.593 | 1.00 | 50.00 | C |
| ATOM | 419 | NZ | LYS | F | 315 | 4.484 | 47.653 | −2.809 | 1.00 | 50.00 | N |
| ATOM | 420 | N | PHE | F | 352 | 7.473 | 57.258 | −2.585 | 1.00 | 50.00 | N |
| ATOM | 421 | CA | PHE | F | 352 | 7.268 | 56.495 | −1.351 | 1.00 | 50.00 | C |
| ATOM | 422 | C | PHE | F | 352 | 6.140 | 55.484 | −1.523 | 1.00 | 50.00 | C |
| ATOM | 423 | O | PHE | F | 352 | 4.997 | 55.904 | −1.350 | 1.00 | 50.00 | O |
| ATOM | 424 | CB | PHE | F | 352 | 6.991 | 57.287 | −0.070 | 1.00 | 50.00 | C |
| ATOM | 425 | CG | PHE | F | 352 | 6.696 | 56.524 | 1.206 | 1.00 | 50.00 | C |
| ATOM | 426 | CD1 | PHE | F | 352 | 7.686 | 55.752 | 1.695 | 1.00 | 50.00 | C |
| ATOM | 427 | CD2 | PHE | F | 352 | 5.505 | 56.537 | 1.839 | 1.00 | 50.00 | C |
| ATOM | 428 | CE1 | PHE | F | 352 | 7.516 | 55.030 | 2.820 | 1.00 | 50.00 | C |
| ATOM | 429 | CE2 | PHE | F | 352 | 5.323 | 55.813 | 2.963 | 1.00 | 50.00 | C |
| ATOM | 430 | CZ | PHE | F | 352 | 6.329 | 55.065 | 3.458 | 1.00 | 50.00 | C |
| ATOM | 431 | N | PRO | F | 353 | 6.335 | 54.184 | −1.682 | 1.00 | 50.00 | N |
| ATOM | 432 | CA | PRO | F | 353 | 5.296 | 53.160 | −1.600 | 1.00 | 50.00 | C |
| ATOM | 433 | C | PRO | F | 353 | 4.779 | 52.996 | −0.176 | 1.00 | 50.00 | C |
| ATOM | 434 | O | PRO | F | 353 | 5.503 | 52.752 | 0.789 | 1.00 | 50.00 | O |
| ATOM | 435 | CB | PRO | F | 353 | 5.999 | 51.904 | −2.114 | 1.00 | 50.00 | C |
| ATOM | 436 | CG | PRO | F | 353 | 7.456 | 52.091 | −1.698 | 1.00 | 50.00 | C |
| ATOM | 437 | CD | PRO | F | 353 | 7.617 | 53.574 | −2.015 | 1.00 | 50.00 | C |
| ATOM | 438 | N | GLN | F | 354 | 3.465 | 53.103 | −0.107 | 1.00 | 50.00 | N |
| ATOM | 439 | CA | GLN | F | 354 | 2.629 | 52.622 | 0.990 | 1.00 | 50.00 | C |
| ATOM | 440 | C | GLN | F | 354 | 2.413 | 51.126 | 0.792 | 1.00 | 50.00 | C |
| ATOM | 441 | O | GLN | F | 354 | 1.453 | 50.683 | 0.163 | 1.00 | 50.00 | O |
| ATOM | 442 | CB | GLN | F | 354 | 1.325 | 53.424 | 1.002 | 1.00 | 50.00 | C |
| ATOM | 443 | CG | GLN | F | 354 | 1.447 | 54.920 | 1.291 | 1.00 | 50.00 | C |
| ATOM | 444 | CD | GLN | F | 354 | 1.743 | 55.287 | 2.738 | 1.00 | 50.00 | C |
| ATOM | 445 | OE1 | GLN | F | 354 | 1.911 | 54.448 | 3.622 | 1.00 | 50.00 | O |
| ATOM | 446 | NE2 | GLN | F | 354 | 1.843 | 56.570 | 3.034 | 1.00 | 50.00 | N |
| ATOM | 447 | N | ALA | F | 355 | 3.331 | 50.354 | 1.351 | 1.00 | 50.00 | N |
| ATOM | 448 | CA | ALA | F | 355 | 3.244 | 48.895 | 1.427 | 1.00 | 50.00 | C |
| ATOM | 449 | C | ALA | F | 355 | 2.143 | 48.258 | 2.269 | 1.00 | 50.00 | C |
| ATOM | 450 | O | ALA | F | 355 | 2.025 | 47.033 | 2.247 | 1.00 | 50.00 | O |
| ATOM | 451 | CB | ALA | F | 355 | 4.599 | 48.410 | 1.936 | 1.00 | 50.00 | C |
| ATOM | 452 | N | GLU | F | 356 | 1.312 | 49.029 | 2.951 | 1.00 | 50.00 | N |
| ATOM | 453 | CA | GLU | F | 356 | 0.052 | 48.597 | 3.560 | 1.00 | 50.00 | C |
| ATOM | 454 | C | GLU | F | 356 | −0.887 | 47.724 | 2.735 | 1.00 | 50.00 | C |
| ATOM | 455 | O | GLU | F | 356 | −1.527 | 46.841 | 3.305 | 1.00 | 50.00 | O |
| ATOM | 456 | CB | GLU | F | 356 | −0.689 | 49.831 | 4.079 | 1.00 | 50.00 | C |
| ATOM | 457 | CG | GLU | F | 356 | −0.035 | 50.621 | 5.212 | 1.00 | 50.00 | C |
| ATOM | 458 | CD | GLU | F | 356 | 0.053 | 49.865 | 6.529 | 1.00 | 50.00 | C |
| ATOM | 459 | OE1 | GLU | F | 356 | 1.062 | 49.163 | 6.756 | 1.00 | 50.00 | O |
| ATOM | 460 | OE2 | GLU | F | 356 | −0.891 | 49.956 | 7.343 | 1.00 | 50.00 | O |
| ATOM | 461 | N | THR | F | 357 | −0.950 | 47.915 | 1.427 | 1.00 | 50.00 | N |
| ATOM | 462 | CA | THR | F | 357 | −1.678 | 47.042 | 0.505 | 1.00 | 50.00 | C |
| ATOM | 463 | C | THR | F | 357 | −0.867 | 45.862 | −0.026 | 1.00 | 50.00 | C |
| ATOM | 464 | O | THR | F | 357 | −1.098 | 45.441 | −1.159 | 1.00 | 50.00 | O |
| ATOM | 465 | CB | THR | F | 357 | −2.273 | 47.986 | −0.543 | 1.00 | 50.00 | C |
| ATOM | 466 | OG1 | THR | F | 357 | −3.046 | 49.019 | 0.052 | 1.00 | 50.00 | O |
| ATOM | 467 | CG2 | THR | F | 357 | −3.241 | 47.312 | −1.512 | 1.00 | 50.00 | C |
| ATOM | 468 | N | CYS | F | 358 | 0.042 | 45.249 | 0.718 | 1.00 | 50.00 | N |
| ATOM | 469 | CA | CYS | F | 358 | 0.888 | 44.170 | 0.209 | 1.00 | 50.00 | C |
| ATOM | 470 | C | CYS | F | 358 | 1.270 | 43.147 | 1.273 | 1.00 | 50.00 | C |
| ATOM | 471 | O | CYS | F | 358 | 1.814 | 43.501 | 2.318 | 1.00 | 50.00 | O |
| ATOM | 472 | CB | CYS | F | 358 | 2.133 | 44.739 | −0.473 | 1.00 | 50.00 | C |
| ATOM | 473 | SG | CYS | F | 358 | 1.848 | 46.141 | −1.568 | 1.00 | 50.00 | S |
| ATOM | 474 | N | PHE | F | 366 | 3.532 | 40.808 | −3.222 | 1.00 | 50.00 | N |
| ATOM | 475 | CA | PHE | F | 366 | 2.175 | 40.783 | −3.763 | 1.00 | 50.00 | C |
| ATOM | 476 | C | PHE | F | 366 | 1.500 | 42.102 | −3.409 | 1.00 | 50.00 | C |
| ATOM | 477 | O | PHE | F | 366 | 1.368 | 42.398 | −2.224 | 1.00 | 50.00 | O |
| ATOM | 478 | CB | PHE | F | 366 | 1.537 | 39.473 | −3.289 | 1.00 | 50.00 | C |
| ATOM | 479 | CG | PHE | F | 366 | 0.295 | 39.189 | −2.471 | 1.00 | 50.00 | C |
| ATOM | 480 | CD1 | PHE | F | 366 | −0.008 | 40.103 | −1.531 | 1.00 | 50.00 | C |
| ATOM | 481 | CD2 | PHE | F | 366 | −0.675 | 38.328 | −2.844 | 1.00 | 50.00 | C |
| ATOM | 482 | CE1 | PHE | F | 366 | −1.290 | 40.395 | −1.236 | 1.00 | 50.00 | C |
| ATOM | 483 | CE2 | PHE | F | 366 | −1.964 | 38.568 | −2.529 | 1.00 | 50.00 | C |
| ATOM | 484 | CZ | PHE | F | 366 | −2.271 | 39.632 | −1.759 | 1.00 | 50.00 | C |
| ATOM | 485 | N | CYS | F | 367 | 1.126 | 42.955 | −4.346 | 1.00 | 50.00 | N |
| ATOM | 486 | CA | CYS | F | 367 | 0.252 | 44.089 | −4.048 | 1.00 | 50.00 | C |
| ATOM | 487 | C | CYS | F | 367 | −1.130 | 43.863 | −4.644 | 1.00 | 50.00 | C |
| ATOM | 488 | O | CYS | F | 367 | −1.316 | 43.324 | −5.733 | 1.00 | 50.00 | O |
| ATOM | 489 | CB | CYS | F | 367 | 0.874 | 45.419 | −4.465 | 1.00 | 50.00 | C |
| ATOM | 490 | SG | CYS | F | 367 | 2.336 | 45.751 | −3.470 | 1.00 | 50.00 | S |
| ATOM | 491 | N | ASP | F | 368 | −2.131 | 44.322 | −3.913 | 1.00 | 50.00 | N |
| ATOM | 492 | CA | ASP | F | 368 | −3.466 | 44.600 | −4.444 | 1.00 | 50.00 | C |
| ATOM | 493 | C | ASP | F | 368 | −3.591 | 45.918 | −5.206 | 1.00 | 50.00 | C |
| ATOM | 494 | O | ASP | F | 368 | −4.398 | 46.007 | −6.131 | 1.00 | 50.00 | O |
| ATOM | 495 | CB | ASP | F | 368 | −4.409 | 44.445 | −3.249 | 1.00 | 50.00 | C |
| ATOM | 496 | CG | ASP | F | 368 | −4.552 | 43.063 | −2.623 | 1.00 | 50.00 | C |
| ATOM | 497 | OD1 | ASP | F | 368 | −4.839 | 42.078 | −3.337 | 1.00 | 50.00 | O |
| ATOM | 498 | OD2 | ASP | F | 368 | −4.347 | 42.944 | −1.396 | 1.00 | 50.00 | O |
| ATOM | 499 | N | THR | F | 369 | −2.817 | 46.934 | −4.858 | 1.00 | 50.00 | N |
| ATOM | 500 | CA | THR | F | 369 | −2.805 | 48.290 | −5.408 | 1.00 | 50.00 | C |
| ATOM | 501 | C | THR | F | 369 | −1.393 | 48.840 | −5.232 | 1.00 | 50.00 | C |
| ATOM | 502 | O | THR | F | 369 | −0.616 | 48.393 | −4.388 | 1.00 | 50.00 | O |
| ATOM | 503 | CB | THR | F | 369 | −3.810 | 49.168 | −4.659 | 1.00 | 50.00 | C |
| ATOM | 504 | OG1 | THR | F | 369 | −5.103 | 48.581 | −4.674 | 1.00 | 50.00 | O |
| ATOM | 505 | CG2 | THR | F | 369 | −3.973 | 50.617 | −5.112 | 1.00 | 50.00 | C |
| ATOM | 506 | N | MET | F | 370 | −1.047 | 49.886 | −5.964 | 1.00 | 50.00 | N |
| ATOM | 507 | CA | MET | F | 370 | 0.242 | 50.567 | −5.827 | 1.00 | 50.00 | C |
| ATOM | 508 | C | MET | F | 370 | 0.087 | 51.971 | −5.263 | 1.00 | 50.00 | C |
| ATOM | 509 | O | MET | F | 370 | 0.114 | 52.994 | −5.946 | 1.00 | 50.00 | O |
| ATOM | 510 | CB | MET | F | 370 | 1.007 | 50.511 | −7.147 | 1.00 | 50.00 | C |
| ATOM | 511 | CG | MET | F | 370 | 1.513 | 49.093 | −7.391 | 1.00 | 50.00 | C |
| ATOM | 512 | SD | MET | F | 370 | 2.636 | 48.508 | −6.111 | 1.00 | 50.00 | S |
| ATOM | 513 | CE | MET | F | 370 | 4.042 | 49.575 | −6.462 | 1.00 | 50.00 | C |
| ATOM | 514 | N | ASN | F | 371 | −0.027 | 51.967 | −3.946 | 1.00 | 50.00 | N |
| ATOM | 515 | CA | ASN | F | 371 | −0.281 | 53.160 | −3.143 | 1.00 | 50.00 | C |
| ATOM | 516 | C | ASN | F | 371 | 1.040 | 53.882 | −2.873 | 1.00 | 50.00 | C |
| ATOM | 517 | O | ASN | F | 371 | 1.843 | 53.257 | −2.184 | 1.00 | 50.00 | O |
| ATOM | 518 | CB | ASN | F | 371 | −1.206 | 52.638 | −2.036 | 1.00 | 50.00 | C |
| ATOM | 519 | CG | ASN | F | 371 | −2.677 | 52.398 | −2.365 | 1.00 | 50.00 | C |
| ATOM | 520 | OD1 | ASN | F | 371 | −3.188 | 52.820 | −3.402 | 1.00 | 50.00 | O |
| ATOM | 521 | ND2 | ASN | F | 371 | −3.491 | 51.809 | −1.509 | 1.00 | 50.00 | N |
| ATOM | 522 | N | SER | F | 372 | 1.393 | 55.034 | −3.428 | 1.00 | 50.00 | N |
| ATOM | 523 | CA | SER | F | 372 | 2.632 | 55.798 | −3.233 | 1.00 | 50.00 | C |
| ATOM | 524 | C | SER | F | 372 | 2.626 | 57.328 | −3.126 | 1.00 | 50.00 | C |
| ATOM | 525 | O | SER | F | 372 | 1.630 | 57.970 | −3.452 | 1.00 | 50.00 | O |
| ATOM | 526 | CB | SER | F | 372 | 3.567 | 55.353 | −4.358 | 1.00 | 50.00 | C |
| ATOM | 527 | OG | SER | F | 372 | 4.880 | 55.882 | −4.242 | 1.00 | 50.00 | O |
| ATOM | 528 | N | LEU | F | 373 | 3.635 | 58.051 | −2.655 | 1.00 | 50.00 | N |
| ATOM | 529 | CA | LEU | F | 373 | 3.615 | 59.485 | −2.338 | 1.00 | 50.00 | C |
| ATOM | 530 | C | LEU | F | 373 | 4.777 | 60.270 | −2.945 | 1.00 | 50.00 | C |
| ATOM | 531 | O | LEU | F | 373 | 5.860 | 59.705 | −3.093 | 1.00 | 50.00 | O |
| ATOM | 532 | CB | LEU | F | 373 | 3.592 | 59.734 | −0.826 | 1.00 | 50.00 | C |
| ATOM | 533 | CG | LEU | F | 373 | 2.297 | 59.582 | −0.027 | 1.00 | 50.00 | C |
| ATOM | 534 | CD1 | LEU | F | 373 | 1.890 | 58.122 | 0.145 | 1.00 | 50.00 | C |
| ATOM | 535 | CD2 | LEU | F | 373 | 2.361 | 60.106 | 1.408 | 1.00 | 50.00 | C |
| END | | | | | | | | | | | |

TABLE 2
List of the PDB format atomic coordinates for all the atoms in the RSV-
F model.

| | | | | | | | | | | | |
|---|---|---|---|---|---|---|---|---|---|---|---|
| ATOM | 1 | N | THR | A | 29 | −24.629 | 51.461 | 3.288 | 1.00 | 50.00 | N |
| ATOM | 2 | CA | THR | A | 29 | −24.243 | 52.668 | 2.538 | 1.00 | 50.00 | C |
| ATOM | 3 | C | THR | A | 29 | −24.836 | 52.713 | 1.135 | 1.00 | 50.00 | C |
| ATOM | 4 | O | THR | A | 29 | −25.220 | 53.811 | 0.733 | 1.00 | 50.00 | O |
| ATOM | 5 | CB | THR | A | 29 | −22.733 | 52.908 | 2.568 | 1.00 | 50.00 | C |
| ATOM | 6 | OG1 | THR | A | 29 | −22.321 | 53.201 | 3.896 | 1.00 | 50.00 | O |
| ATOM | 7 | CG2 | THR | A | 29 | −22.212 | 54.054 | 1.702 | 1.00 | 50.00 | C |
| ATOM | 8 | N | GLU | A | 30 | −24.829 | 51.632 | 0.370 | 1.00 | 50.00 | N |
| ATOM | 9 | CA | GLU | A | 30 | −25.116 | 51.626 | −1.065 | 1.00 | 50.00 | C |
| ATOM | 10 | C | GLU | A | 30 | −23.892 | 52.029 | −1.881 | 1.00 | 50.00 | C |
| ATOM | 11 | O | GLU | A | 30 | −22.780 | 52.208 | −1.385 | 1.00 | 50.00 | O |
| ATOM | 12 | CB | GLU | A | 30 | −26.446 | 52.273 | −1.464 | 1.00 | 50.00 | C |
| ATOM | 13 | CG | GLU | A | 30 | −27.710 | 51.684 | −0.839 | 1.00 | 50.00 | C |
| ATOM | 14 | CD | GLU | A | 30 | −28.147 | 50.398 | −1.525 | 1.00 | 50.00 | C |
| ATOM | 15 | OE1 | GLU | A | 30 | −27.705 | 49.307 | −1.106 | 1.00 | 50.00 | O |
| ATOM | 16 | OE2 | GLU | A | 30 | −28.916 | 50.474 | −2.508 | 1.00 | 50.00 | O |
| ATOM | 17 | N | GLU | A | 31 | −24.104 | 52.086 | −3.184 | 1.00 | 50.00 | N |
| ATOM | 18 | CA | GLU | A | 31 | −23.081 | 52.352 | −4.196 | 1.00 | 50.00 | C |
| ATOM | 19 | C | GLU | A | 31 | −23.480 | 53.436 | −5.189 | 1.00 | 50.00 | C |
| ATOM | 20 | O | GLU | A | 31 | −22.958 | 53.451 | −6.304 | 1.00 | 50.00 | O |
| ATOM | 21 | CB | GLU | A | 31 | −22.842 | 51.028 | −4.922 | 1.00 | 50.00 | C |
| ATOM | 22 | CG | GLU | A | 31 | −22.331 | 49.826 | −4.128 | 1.00 | 50.00 | C |
| ATOM | 23 | CD | GLU | A | 31 | −20.982 | 49.925 | −3.432 | 1.00 | 50.00 | C |
| ATOM | 24 | OE1 | GLU | A | 31 | −20.659 | 50.956 | −2.802 | 1.00 | 50.00 | O |
| ATOM | 25 | OE2 | GLU | A | 31 | −20.218 | 48.939 | −3.513 | 1.00 | 50.00 | O |
| ATOM | 26 | N | PHE | A | 32 | −24.278 | 54.422 | −4.809 | 1.00 | 50.00 | N |
| ATOM | 27 | CA | PHE | A | 32 | −24.277 | 55.704 | −5.511 | 1.00 | 50.00 | C |
| ATOM | 28 | C | PHE | A | 32 | −22.961 | 56.454 | −5.323 | 1.00 | 50.00 | C |
| ATOM | 29 | O | PHE | A | 32 | −22.430 | 56.980 | −6.301 | 1.00 | 50.00 | O |
| ATOM | 30 | CB | PHE | A | 32 | −25.570 | 56.492 | −5.287 | 1.00 | 50.00 | C |
| ATOM | 31 | CG | PHE | A | 32 | −25.882 | 57.395 | −6.462 | 1.00 | 50.00 | C |
| ATOM | 32 | CD1 | PHE | A | 32 | −26.914 | 57.244 | −7.318 | 1.00 | 50.00 | C |
| ATOM | 33 | CD2 | PHE | A | 32 | −24.977 | 58.361 | −6.714 | 1.00 | 50.00 | C |
| ATOM | 34 | CE1 | PHE | A | 32 | −27.083 | 58.106 | −8.343 | 1.00 | 50.00 | C |
| ATOM | 35 | CE2 | PHE | A | 32 | −25.089 | 59.177 | −7.780 | 1.00 | 50.00 | C |
| ATOM | 36 | CZ | PHE | A | 32 | −26.188 | 59.092 | −8.553 | 1.00 | 50.00 | C |
| ATOM | 37 | N | TYR | A | 33 | −22.366 | 56.446 | −4.142 | 1.00 | 50.00 | N |
| ATOM | 38 | CA | TYR | A | 33 | −20.962 | 56.802 | −3.942 | 1.00 | 50.00 | C |
| ATOM | 39 | C | TYR | A | 33 | −19.843 | 56.187 | −4.779 | 1.00 | 50.00 | C |
| ATOM | 40 | O | TYR | A | 33 | −18.725 | 56.700 | −4.776 | 1.00 | 50.00 | O |
| ATOM | 41 | CB | TYR | A | 33 | −20.762 | 56.706 | −2.427 | 1.00 | 50.00 | C |
| ATOM | 42 | CG | TYR | A | 33 | −19.468 | 57.300 | −1.919 | 1.00 | 50.00 | C |
| ATOM | 43 | CD1 | TYR | A | 33 | −19.050 | 58.459 | −2.466 | 1.00 | 50.00 | C |
| ATOM | 44 | CD2 | TYR | A | 33 | −18.643 | 56.624 | −1.094 | 1.00 | 50.00 | C |
| ATOM | 45 | CE1 | TYR | A | 33 | −17.752 | 58.809 | −2.389 | 1.00 | 50.00 | C |
| ATOM | 46 | CE2 | TYR | A | 33 | −17.424 | 57.127 | −0.811 | 1.00 | 50.00 | C |
| ATOM | 47 | CZ | TYR | A | 33 | −16.941 | 58.159 | −1.533 | 1.00 | 50.00 | C |
| ATOM | 48 | OH | TYR | A | 33 | −15.696 | 58.695 | −1.327 | 1.00 | 50.00 | O |
| ATOM | 49 | N | GLN | A | 34 | −20.106 | 55.166 | −5.576 | 1.00 | 50.00 | N |
| ATOM | 50 | CA | GLN | A | 34 | −19.241 | 54.749 | −6.681 | 1.00 | 50.00 | C |
| ATOM | 51 | C | GLN | A | 34 | −19.004 | 55.767 | −7.791 | 1.00 | 50.00 | C |
| ATOM | 52 | O | GLN | A | 34 | −17.853 | 55.950 | −8.185 | 1.00 | 50.00 | O |
| ATOM | 53 | CB | GLN | A | 34 | −19.797 | 53.460 | −7.285 | 1.00 | 50.00 | C |
| ATOM | 54 | CG | GLN | A | 34 | −19.799 | 52.244 | −6.361 | 1.00 | 50.00 | C |
| ATOM | 55 | CD | GLN | A | 34 | −18.437 | 51.663 | −6.015 | 1.00 | 50.00 | C |
| ATOM | 56 | OE1 | GLN | A | 34 | −17.392 | 51.971 | −6.587 | 1.00 | 50.00 | O |
| ATOM | 57 | NE2 | GLN | A | 34 | −18.454 | 50.783 | −5.032 | 1.00 | 50.00 | N |
| ATOM | 58 | N | SER | A | 35 | −20.051 | 56.424 | −8.264 | 1.00 | 50.00 | N |
| ATOM | 59 | CA | SER | A | 35 | −19.978 | 57.622 | −9.102 | 1.00 | 50.00 | C |
| ATOM | 60 | C | SER | A | 35 | −19.321 | 58.879 | −8.545 | 1.00 | 50.00 | C |
| ATOM | 61 | O | SER | A | 35 | −18.995 | 59.783 | −9.314 | 1.00 | 50.00 | O |
| ATOM | 62 | CB | SER | A | 35 | −21.396 | 58.071 | −9.450 | 1.00 | 50.00 | C |
| ATOM | 63 | OG | SER | A | 35 | −22.106 | 57.095 | −10.198 | 1.00 | 50.00 | O |
| ATOM | 64 | N | THR | A | 36 | −19.172 | 58.980 | −7.236 | 1.00 | 50.00 | N |
| ATOM | 65 | CA | THR | A | 36 | −18.287 | 59.954 | −6.599 | 1.00 | 50.00 | C |
| ATOM | 66 | C | THR | A | 36 | −16.887 | 59.417 | −6.313 | 1.00 | 50.00 | C |
| ATOM | 67 | O | THR | A | 36 | −16.045 | 60.248 | −5.976 | 1.00 | 50.00 | O |
| ATOM | 68 | CB | THR | A | 36 | −19.097 | 60.481 | −5.414 | 1.00 | 50.00 | C |
| ATOM | 69 | OG1 | THR | A | 36 | −20.313 | 61.113 | −5.785 | 1.00 | 50.00 | O |
| ATOM | 70 | CG2 | THR | A | 36 | −18.372 | 61.524 | −4.570 | 1.00 | 50.00 | C |
| ATOM | 71 | N | CYS | A | 37 | −16.543 | 58.156 | −6.529 | 1.00 | 50.00 | N |
| ATOM | 72 | CA | CYS | A | 37 | −15.183 | 57.631 | −6.670 | 1.00 | 50.00 | C |
| ATOM | 73 | C | CYS | A | 37 | −14.567 | 56.857 | −5.510 | 1.00 | 50.00 | C |
| ATOM | 74 | O | CYS | A | 37 | −13.353 | 56.661 | −5.463 | 1.00 | 50.00 | O |
| ATOM | 75 | CB | CYS | A | 37 | −14.192 | 58.604 | −7.304 | 1.00 | 50.00 | C |
| ATOM | 76 | SG | CYS | A | 37 | −14.982 | 59.610 | −8.573 | 1.00 | 50.00 | S |
| ATOM | 77 | N | SER | A | 38 | −15.395 | 56.317 | −4.632 | 1.00 | 50.00 | N |
| ATOM | 78 | CA | SER | A | 38 | −15.007 | 55.240 | −3.723 | 1.00 | 50.00 | C |
| ATOM | 79 | C | SER | A | 38 | −15.810 | 53.972 | −3.971 | 1.00 | 50.00 | C |
| ATOM | 80 | O | SER | A | 38 | −17.011 | 53.917 | −4.233 | 1.00 | 50.00 | O |
| ATOM | 81 | CB | SER | A | 38 | −15.290 | 55.565 | −2.261 | 1.00 | 50.00 | C |
| ATOM | 82 | OG | SER | A | 38 | −14.390 | 56.532 | −1.743 | 1.00 | 50.00 | O |
| ATOM | 83 | N | ALA | A | 39 | −15.019 | 52.961 | −3.666 | 1.00 | 50.00 | N |
| ATOM | 84 | CA | ALA | A | 39 | −15.383 | 51.551 | −3.731 | 1.00 | 50.00 | C |
| ATOM | 85 | C | ALA | A | 39 | −15.577 | 51.048 | −2.308 | 1.00 | 50.00 | C |
| ATOM | 86 | O | ALA | A | 39 | −14.678 | 51.151 | −1.473 | 1.00 | 50.00 | O |
| ATOM | 87 | CB | ALA | A | 39 | −14.256 | 50.968 | −4.581 | 1.00 | 50.00 | C |
| ATOM | 88 | N | VAL | A | 40 | −16.739 | 50.476 | −2.038 | 1.00 | 50.00 | N |
| ATOM | 89 | CA | VAL | A | 40 | −16.959 | 49.650 | −0.850 | 1.00 | 50.00 | C |
| ATOM | 90 | C | VAL | A | 40 | −16.279 | 48.325 | −1.172 | 1.00 | 50.00 | C |
| ATOM | 91 | O | VAL | A | 40 | −16.786 | 47.519 | −1.952 | 1.00 | 50.00 | O |
| ATOM | 92 | CB | VAL | A | 40 | −18.431 | 49.521 | −0.447 | 1.00 | 50.00 | C |
| ATOM | 93 | CG1 | VAL | A | 40 | −18.731 | 48.435 | 0.586 | 1.00 | 50.00 | C |
| ATOM | 94 | CG2 | VAL | A | 40 | −19.013 | 50.832 | 0.081 | 1.00 | 50.00 | C |
| ATOM | 95 | N | SER | A | 41 | −15.135 | 48.109 | −0.543 | 1.00 | 50.00 | N |
| ATOM | 96 | CA | SER | A | 41 | −14.484 | 46.800 | −0.522 | 1.00 | 50.00 | C |
| ATOM | 97 | C | SER | A | 41 | −15.320 | 45.766 | 0.223 | 1.00 | 50.00 | C |
| ATOM | 98 | O | SER | A | 41 | −15.592 | 44.724 | −0.372 | 1.00 | 50.00 | O |
| ATOM | 99 | CB | SER | A | 41 | −13.061 | 46.905 | 0.025 | 1.00 | 50.00 | C |
| ATOM | 100 | OG | SER | A | 41 | −12.263 | 47.802 | −0.735 | 1.00 | 50.00 | O |
| ATOM | 101 | N | LYS | A | 42 | −15.715 | 46.008 | 1.464 | 1.00 | 50.00 | N |
| ATOM | 102 | CA | LYS | A | 42 | −16.327 | 44.992 | 2.321 | 1.00 | 50.00 | C |
| ATOM | 103 | C | LYS | A | 42 | −17.301 | 45.617 | 3.310 | 1.00 | 50.00 | C |
| ATOM | 104 | O | LYS | A | 42 | −17.044 | 46.671 | 3.891 | 1.00 | 50.00 | O |
| ATOM | 105 | CB | LYS | A | 42 | −15.272 | 44.162 | 3.055 | 1.00 | 50.00 | C |
| ATOM | 106 | CG | LYS | A | 42 | −14.479 | 43.268 | 2.103 | 1.00 | 50.00 | C |
| ATOM | 107 | CD | LYS | A | 42 | −13.490 | 42.291 | 2.732 | 1.00 | 50.00 | C |
| ATOM | 108 | CE | LYS | A | 42 | −12.762 | 41.546 | 1.615 | 1.00 | 50.00 | C |
| ATOM | 109 | NZ | LYS | A | 42 | −11.797 | 40.590 | 2.175 | 1.00 | 50.00 | N |
| ATOM | 110 | N | GLY | A | 43 | −18.402 | 44.922 | 3.540 | 1.00 | 50.00 | N |
| ATOM | 111 | CA | GLY | A | 43 | −19.300 | 45.185 | 4.663 | 1.00 | 50.00 | C |
| ATOM | 112 | C | GLY | A | 43 | −19.280 | 43.986 | 5.599 | 1.00 | 50.00 | C |
| ATOM | 113 | O | GLY | A | 43 | −19.303 | 42.820 | 5.207 | 1.00 | 50.00 | O |
| ATOM | 114 | N | TYR | A | 44 | −19.221 | 44.333 | 6.872 | 1.00 | 50.00 | N |
| ATOM | 115 | CA | TYR | A | 44 | −19.060 | 43.387 | 7.972 | 1.00 | 50.00 | C |
| ATOM | 116 | C | TYR | A | 44 | −19.972 | 43.812 | 9.118 | 1.00 | 50.00 | C |
| ATOM | 117 | O | TYR | A | 44 | −20.299 | 44.982 | 9.291 | 1.00 | 50.00 | O |
| ATOM | 118 | CB | TYR | A | 44 | −17.572 | 43.269 | 8.313 | 1.00 | 50.00 | C |
| ATOM | 119 | CG | TYR | A | 44 | −17.327 | 42.317 | 9.463 | 1.00 | 50.00 | C |
| ATOM | 120 | CD1 | TYR | A | 44 | −17.853 | 41.084 | 9.316 | 1.00 | 50.00 | C |
| ATOM | 121 | CD2 | TYR | A | 44 | −17.259 | 42.872 | 10.691 | 1.00 | 50.00 | C |
| ATOM | 122 | CE1 | TYR | A | 44 | −18.540 | 40.548 | 10.345 | 1.00 | 50.00 | C |
| ATOM | 123 | CE2 | TYR | A | 44 | −17.719 | 42.192 | 11.760 | 1.00 | 50.00 | C |
| ATOM | 124 | CZ | TYR | A | 44 | −18.384 | 41.035 | 11.592 | 1.00 | 50.00 | C |
| ATOM | 125 | OH | TYR | A | 44 | −18.760 | 40.323 | 12.701 | 1.00 | 50.00 | O |
| ATOM | 126 | N | LEU | A | 45 | −20.342 | 42.863 | 9.959 | 1.00 | 50.00 | N |
| ATOM | 127 | CA | LEU | A | 45 | −21.007 | 42.992 | 11.255 | 1.00 | 50.00 | C |
| ATOM | 128 | C | LEU | A | 45 | −20.257 | 43.625 | 12.426 | 1.00 | 50.00 | C |
| ATOM | 129 | O | LEU | A | 45 | −19.450 | 44.501 | 12.118 | 1.00 | 50.00 | O |
| ATOM | 130 | CB | LEU | A | 45 | −21.706 | 41.634 | 11.338 | 1.00 | 50.00 | C |
| ATOM | 131 | CG | LEU | A | 45 | −22.865 | 41.301 | 10.399 | 1.00 | 50.00 | C |
| ATOM | 132 | CD1 | LEU | A | 45 | −22.613 | 41.196 | 8.894 | 1.00 | 50.00 | C |
| ATOM | 133 | CD2 | LEU | A | 45 | −23.852 | 40.208 | 10.790 | 1.00 | 50.00 | C |
| ATOM | 134 | N | SER | A | 46 | −20.430 | 43.306 | 13.701 | 1.00 | 50.00 | N |
| ATOM | 135 | CA | SER | A | 46 | −19.534 | 43.693 | 14.797 | 1.00 | 50.00 | C |
| ATOM | 136 | C | SER | A | 46 | −19.945 | 43.096 | 16.136 | 1.00 | 50.00 | C |
| ATOM | 137 | O | SER | A | 46 | −21.138 | 42.950 | 16.405 | 1.00 | 50.00 | O |
| ATOM | 138 | CB | SER | A | 46 | −19.432 | 45.178 | 15.145 | 1.00 | 50.00 | C |
| ATOM | 139 | OG | SER | A | 46 | −18.767 | 45.918 | 14.135 | 1.00 | 50.00 | O |
| ATOM | 140 | N | ALA | A | 47 | −18.982 | 42.808 | 17.000 | 1.00 | 50.00 | N |
| ATOM | 141 | CA | ALA | A | 47 | −19.235 | 42.104 | 18.258 | 1.00 | 50.00 | C |
| ATOM | 142 | C | ALA | A | 47 | −18.051 | 41.862 | 19.188 | 1.00 | 50.00 | C |
| ATOM | 143 | O | ALA | A | 47 | −16.889 | 41.927 | 18.788 | 1.00 | 50.00 | O |
| ATOM | 144 | CB | ALA | A | 47 | −19.732 | 40.735 | 17.810 | 1.00 | 50.00 | C |
| ATOM | 145 | N | LEU | A | 48 | −18.362 | 41.602 | 20.448 | 1.00 | 50.00 | N |
| ATOM | 146 | CA | LEU | A | 48 | −17.446 | 41.727 | 21.584 | 1.00 | 50.00 | C |
| ATOM | 147 | C | LEU | A | 48 | −17.209 | 40.373 | 22.239 | 1.00 | 50.00 | C |
| ATOM | 148 | O | LEU | A | 48 | −18.122 | 39.555 | 22.342 | 1.00 | 50.00 | O |
| ATOM | 149 | CB | LEU | A | 48 | −17.957 | 42.719 | 22.630 | 1.00 | 50.00 | C |
| ATOM | 150 | CG | LEU | A | 48 | −17.826 | 44.212 | 22.327 | 1.00 | 50.00 | C |
| ATOM | 151 | CD1 | LEU | A | 48 | −18.481 | 44.627 | 21.013 | 1.00 | 50.00 | C |
| ATOM | 152 | CD2 | LEU | A | 48 | −18.467 | 45.099 | 23.393 | 1.00 | 50.00 | C |
| ATOM | 153 | N | ARG | A | 49 | −15.984 | 40.135 | 22.680 | 1.00 | 50.00 | N |
| ATOM | 154 | CA | ARG | A | 49 | −15.522 | 38.795 | 23.046 | 1.00 | 50.00 | C |
| ATOM | 155 | C | ARG | A | 49 | −14.573 | 38.799 | 24.235 | 1.00 | 50.00 | C |
| ATOM | 156 | O | ARG | A | 49 | −13.971 | 39.808 | 24.603 | 1.00 | 50.00 | O |
| ATOM | 157 | CB | ARG | A | 49 | −14.786 | 38.075 | 21.916 | 1.00 | 50.00 | C |
| ATOM | 158 | CG | ARG | A | 49 | −15.658 | 37.981 | 20.671 | 1.00 | 50.00 | C |
| ATOM | 159 | CD | ARG | A | 49 | −15.166 | 36.915 | 19.703 | 1.00 | 50.00 | C |
| ATOM | 160 | NE | ARG | A | 49 | −15.842 | 37.186 | 18.436 | 1.00 | 50.00 | N |
| ATOM | 161 | CZ | ARG | A | 49 | −15.715 | 36.495 | 17.319 | 1.00 | 50.00 | C |
| ATOM | 162 | NH1 | ARG | A | 49 | −14.965 | 35.416 | 17.324 | 1.00 | 50.00 | N |
| ATOM | 163 | NH2 | ARG | A | 49 | −16.254 | 36.887 | 16.179 | 1.00 | 50.00 | N |
| ATOM | 164 | N | THR | A | 50 | −14.426 | 37.602 | 24.777 | 1.00 | 50.00 | N |
| ATOM | 165 | CA | THR | A | 50 | −13.496 | 37.333 | 25.871 | 1.00 | 50.00 | C |
| ATOM | 166 | C | THR | A | 50 | −13.054 | 35.878 | 25.783 | 1.00 | 50.00 | C |
| ATOM | 167 | O | THR | A | 50 | −13.905 | 34.991 | 25.823 | 1.00 | 50.00 | O |
| ATOM | 168 | CB | THR | A | 50 | −14.247 | 37.553 | 27.183 | 1.00 | 50.00 | C |
| ATOM | 169 | OG1 | THR | A | 50 | −14.969 | 38.776 | 27.206 | 1.00 | 50.00 | O |
| ATOM | 170 | CG2 | THR | A | 50 | −13.341 | 37.426 | 28.404 | 1.00 | 50.00 | C |
| ATOM | 171 | N | GLY | A | 51 | −11.754 | 35.644 | 25.853 | 1.00 | 50.00 | N |
| ATOM | 172 | CA | GLY | A | 51 | −11.189 | 34.306 | 26.025 | 1.00 | 50.00 | C |
| ATOM | 173 | C | GLY | A | 51 | −11.142 | 33.814 | 27.466 | 1.00 | 50.00 | C |
| ATOM | 174 | O | GLY | A | 51 | −10.108 | 33.854 | 28.131 | 1.00 | 50.00 | O |
| ATOM | 175 | N | TRP | A | 52 | −12.296 | 33.351 | 27.913 | 1.00 | 50.00 | N |
| ATOM | 176 | CA | TRP | A | 52 | −12.609 | 33.085 | 29.317 | 1.00 | 50.00 | C |
| ATOM | 177 | C | TRP | A | 52 | −13.147 | 31.675 | 29.539 | 1.00 | 50.00 | C |
| ATOM | 178 | O | TRP | A | 52 | −12.739 | 31.073 | 30.533 | 1.00 | 50.00 | O |
| ATOM | 179 | CB | TRP | A | 52 | −13.547 | 34.229 | 29.701 | 1.00 | 50.00 | C |
| ATOM | 180 | CG | TRP | A | 52 | −14.293 | 34.145 | 31.011 | 1.00 | 50.00 | C |
| ATOM | 181 | CD1 | TRP | A | 52 | −13.789 | 34.362 | 32.288 | 1.00 | 50.00 | C |
| ATOM | 182 | CD2 | TRP | A | 52 | −15.329 | 33.245 | 31.185 | 1.00 | 50.00 | C |
| ATOM | 183 | NE1 | TRP | A | 52 | −14.307 | 33.479 | 33.214 | 1.00 | 50.00 | N |
| ATOM | 184 | CE2 | TRP | A | 52 | −15.170 | 32.713 | 32.461 | 1.00 | 50.00 | C |
| ATOM | 185 | CE3 | TRP | A | 52 | −16.227 | 32.712 | 30.328 | 1.00 | 50.00 | C |
| ATOM | 186 | CZ2 | TRP | A | 52 | −15.823 | 31.577 | 32.772 | 1.00 | 50.00 | C |
| ATOM | 187 | CZ3 | TRP | A | 52 | −17.027 | 31.713 | 30.745 | 1.00 | 50.00 | C |
| ATOM | 188 | CH2 | TRP | A | 52 | −16.838 | 31.196 | 31.972 | 1.00 | 50.00 | C |
| ATOM | 189 | N | TYR | A | 53 | −14.040 | 31.137 | 28.721 | 1.00 | 50.00 | N |
| ATOM | 190 | CA | TYR | A | 53 | −14.452 | 29.732 | 28.751 | 1.00 | 50.00 | C |
| ATOM | 191 | C | TYR | A | 53 | −13.216 | 28.871 | 28.517 | 1.00 | 50.00 | C |
| ATOM | 192 | O | TYR | A | 53 | −12.203 | 29.250 | 27.929 | 1.00 | 50.00 | O |
| ATOM | 193 | CB | TYR | A | 53 | −15.614 | 29.349 | 27.828 | 1.00 | 50.00 | C |
| ATOM | 194 | CG | TYR | A | 53 | −16.165 | 27.937 | 27.852 | 1.00 | 50.00 | C |
| ATOM | 195 | CD1 | TYR | A | 53 | −17.204 | 27.614 | 28.650 | 1.00 | 50.00 | C |
| ATOM | 196 | CD2 | TYR | A | 53 | −15.564 | 26.956 | 27.148 | 1.00 | 50.00 | C |
| ATOM | 197 | CE1 | TYR | A | 53 | −17.597 | 26.332 | 28.793 | 1.00 | 50.00 | C |
| ATOM | 198 | CE2 | TYR | A | 53 | −15.975 | 25.676 | 27.250 | 1.00 | 50.00 | C |
| ATOM | 199 | CZ | TYR | A | 53 | −16.981 | 25.360 | 28.090 | 1.00 | 50.00 | C |
| ATOM | 200 | OH | TYR | A | 53 | −17.328 | 24.044 | 28.242 | 1.00 | 50.00 | O |
| ATOM | 201 | N | THR | A | 54 | −13.306 | 27.704 | 29.125 | 1.00 | 50.00 | N |
| ATOM | 202 | CA | THR | A | 54 | −12.188 | 26.781 | 29.300 | 1.00 | 50.00 | C |
| ATOM | 203 | C | THR | A | 54 | −12.793 | 25.383 | 29.327 | 1.00 | 50.00 | C |
| ATOM | 204 | O | THR | A | 54 | −13.877 | 25.122 | 29.847 | 1.00 | 50.00 | O |
| ATOM | 205 | CB | THR | A | 54 | −11.350 | 27.169 | 30.520 | 1.00 | 50.00 | C |
| ATOM | 206 | OG1 | THR | A | 54 | −10.446 | 26.120 | 30.834 | 1.00 | 50.00 | O |
| ATOM | 207 | CG2 | THR | A | 54 | −12.103 | 27.548 | 31.795 | 1.00 | 50.00 | C |
| ATOM | 208 | N | SER | A | 55 | −12.025 | 24.475 | 28.751 | 1.00 | 50.00 | N |
| ATOM | 209 | CA | SER | A | 55 | −12.307 | 23.041 | 28.768 | 1.00 | 50.00 | C |
| ATOM | 210 | C | SER | A | 55 | −11.020 | 22.227 | 28.693 | 1.00 | 50.00 | C |
| ATOM | 211 | O | SER | A | 55 | −9.919 | 22.747 | 28.511 | 1.00 | 50.00 | O |
| ATOM | 212 | CB | SER | A | 55 | −13.316 | 22.691 | 27.675 | 1.00 | 50.00 | C |
| ATOM | 213 | OG | SER | A | 55 | −14.637 | 23.060 | 28.038 | 1.00 | 50.00 | O |
| ATOM | 214 | N | VAL | A | 56 | −11.166 | 20.922 | 28.858 | 1.00 | 50.00 | N |
| ATOM | 215 | CA | VAL | A | 56 | −10.075 | 19.947 | 28.826 | 1.00 | 50.00 | C |
| ATOM | 216 | C | VAL | A | 56 | −10.550 | 18.736 | 28.027 | 1.00 | 50.00 | C |
| ATOM | 217 | O | VAL | A | 56 | −11.654 | 18.222 | 28.202 | 1.00 | 50.00 | O |
| ATOM | 218 | CB | VAL | A | 56 | −9.565 | 19.645 | 30.238 | 1.00 | 50.00 | C |
| ATOM | 219 | CG1 | VAL | A | 56 | −8.556 | 18.500 | 30.325 | 1.00 | 50.00 | C |
| ATOM | 220 | CG2 | VAL | A | 56 | −8.889 | 20.855 | 30.885 | 1.00 | 50.00 | C |
| ATOM | 221 | N | ILE | A | 57 | −9.663 | 18.252 | 27.173 | 1.00 | 50.00 | N |
| ATOM | 222 | CA | ILE | A | 57 | −9.725 | 16.978 | 26.454 | 1.00 | 50.00 | C |
| ATOM | 223 | C | ILE | A | 57 | −8.539 | 16.198 | 27.014 | 1.00 | 50.00 | C |
| ATOM | 224 | O | ILE | A | 57 | −7.392 | 16.635 | 26.926 | 1.00 | 50.00 | O |
| ATOM | 225 | CB | ILE | A | 57 | −9.676 | 17.123 | 24.928 | 1.00 | 50.00 | C |
| ATOM | 226 | CG1 | ILE | A | 57 | −10.907 | 17.779 | 24.299 | 1.00 | 50.00 | C |
| ATOM | 227 | CG2 | ILE | A | 57 | −9.307 | 15.853 | 24.160 | 1.00 | 50.00 | C |
| ATOM | 228 | CD1 | ILE | A | 57 | −10.843 | 17.986 | 22.785 | 1.00 | 50.00 | C |
| ATOM | 229 | N | THR | A | 58 | −8.805 | 14.998 | 27.500 | 1.00 | 50.00 | N |
| ATOM | 230 | CA | THR | A | 58 | −7.800 | 14.060 | 28.000 | 1.00 | 50.00 | C |
| ATOM | 231 | C | THR | A | 58 | −7.706 | 12.885 | 27.033 | 1.00 | 50.00 | C |
| ATOM | 232 | O | THR | A | 58 | −8.531 | 11.975 | 27.080 | 1.00 | 50.00 | O |
| ATOM | 233 | CB | THR | A | 58 | −8.156 | 13.629 | 29.424 | 1.00 | 50.00 | C |
| ATOM | 234 | OG1 | THR | A | 58 | −8.007 | 14.735 | 30.303 | 1.00 | 50.00 | O |
| ATOM | 235 | CG2 | THR | A | 58 | −7.329 | 12.472 | 29.983 | 1.00 | 50.00 | C |
| ATOM | 236 | N | ILE | A | 59 | −6.651 | 12.819 | 26.239 | 1.00 | 50.00 | N |
| ATOM | 237 | CA | ILE | A | 59 | −6.382 | 11.712 | 25.319 | 1.00 | 50.00 | C |
| ATOM | 238 | C | ILE | A | 59 | −5.752 | 10.535 | 26.053 | 1.00 | 50.00 | C |
| ATOM | 239 | O | ILE | A | 59 | −4.939 | 10.661 | 26.968 | 1.00 | 50.00 | O |
| ATOM | 240 | CB | ILE | A | 59 | −5.640 | 12.187 | 24.068 | 1.00 | 50.00 | C |
| ATOM | 241 | CG1 | ILE | A | 59 | −6.465 | 13.198 | 23.270 | 1.00 | 50.00 | C |
| ATOM | 242 | CG2 | ILE | A | 59 | −5.195 | 11.110 | 23.079 | 1.00 | 50.00 | C |
| ATOM | 243 | CD1 | ILE | A | 59 | −5.584 | 14.203 | 22.540 | 1.00 | 50.00 | C |
| ATOM | 244 | N | GLU | A | 60 | −6.176 | 9.370 | 25.596 | 1.00 | 50.00 | N |
| ATOM | 245 | CA | GLU | A | 60 | −5.668 | 8.065 | 26.012 | 1.00 | 50.00 | C |
| ATOM | 246 | C | GLU | A | 60 | −5.115 | 7.454 | 24.730 | 1.00 | 50.00 | C |
| ATOM | 247 | O | GLU | A | 60 | −5.809 | 7.145 | 23.762 | 1.00 | 50.00 | O |
| ATOM | 248 | CB | GLU | A | 60 | −6.792 | 7.229 | 26.622 | 1.00 | 50.00 | C |
| ATOM | 249 | CG | GLU | A | 60 | −7.427 | 7.699 | 27.929 | 1.00 | 50.00 | C |
| ATOM | 250 | CD | GLU | A | 60 | −6.603 | 7.604 | 29.202 | 1.00 | 50.00 | C |
| ATOM | 251 | OE1 | GLU | A | 60 | −6.238 | 8.669 | 29.746 | 1.00 | 50.00 | O |
| ATOM | 252 | OE2 | GLU | A | 60 | −6.365 | 6.479 | 29.692 | 1.00 | 50.00 | O |
| ATOM | 253 | N | LEU | A | 61 | −3.800 | 7.337 | 24.746 | 1.00 | 50.00 | N |
| ATOM | 254 | CA | LEU | A | 61 | −2.981 | 6.903 | 23.614 | 1.00 | 50.00 | C |
| ATOM | 255 | C | LEU | A | 61 | −2.904 | 5.409 | 23.328 | 1.00 | 50.00 | C |
| ATOM | 256 | O | LEU | A | 61 | −2.849 | 5.026 | 22.160 | 1.00 | 50.00 | O |
| ATOM | 257 | CB | LEU | A | 61 | −1.573 | 7.421 | 23.910 | 1.00 | 50.00 | C |
| ATOM | 258 | CG | LEU | A | 61 | −1.376 | 8.923 | 24.113 | 1.00 | 50.00 | C |
| ATOM | 259 | CD1 | LEU | A | 61 | −0.001 | 9.152 | 24.735 | 1.00 | 50.00 | C |
| ATOM | 260 | CD2 | LEU | A | 61 | −1.737 | 9.550 | 22.768 | 1.00 | 50.00 | C |
| ATOM | 261 | N | SER | A | 62 | −2.860 | 4.595 | 24.369 | 1.00 | 50.00 | N |
| ATOM | 262 | CA | SER | A | 62 | −2.811 | 3.138 | 24.259 | 1.00 | 50.00 | C |
| ATOM | 263 | C | SER | A | 62 | −4.250 | 2.639 | 24.210 | 1.00 | 50.00 | C |
| ATOM | 264 | O | SER | A | 62 | −4.827 | 2.666 | 23.123 | 1.00 | 50.00 | O |
| ATOM | 265 | CB | SER | A | 62 | −1.901 | 2.602 | 25.364 | 1.00 | 50.00 | C |
| ATOM | 266 | OG | SER | A | 62 | −0.553 | 3.003 | 25.168 | 1.00 | 50.00 | O |
| ATOM | 267 | N | ASN | A | 63 | −4.824 | 2.231 | 25.331 | 1.00 | 50.00 | N |
| ATOM | 268 | CA | ASN | A | 63 | −6.180 | 1.692 | 25.450 | 1.00 | 50.00 | C |
| ATOM | 269 | C | ASN | A | 63 | −6.378 | 0.226 | 25.089 | 1.00 | 50.00 | C |
| ATOM | 270 | O | ASN | A | 63 | −7.157 | −0.465 | 25.744 | 1.00 | 50.00 | O |
| ATOM | 271 | CB | ASN | A | 63 | −7.331 | 2.448 | 24.786 | 1.00 | 50.00 | C |
| ATOM | 272 | CG | ASN | A | 63 | −7.614 | 3.810 | 25.399 | 1.00 | 50.00 | C |
| ATOM | 273 | OD1 | ASN | A | 63 | −8.078 | 3.939 | 26.531 | 1.00 | 50.00 | O |
| ATOM | 274 | ND2 | ASN | A | 63 | −7.338 | 4.843 | 24.627 | 1.00 | 50.00 | N |
| ATOM | 275 | N | ILE | A | 64 | −5.774 | −0.204 | 23.994 | 1.00 | 50.00 | N |
| ATOM | 276 | CA | ILE | A | 64 | −6.077 | −1.472 | 23.334 | 1.00 | 50.00 | C |
| ATOM | 277 | C | ILE | A | 64 | −5.425 | −2.560 | 24.184 | 1.00 | 50.00 | C |
| ATOM | 278 | O | ILE | A | 64 | −4.216 | −2.570 | 24.410 | 1.00 | 50.00 | O |
| ATOM | 279 | CB | ILE | A | 64 | −5.672 | −1.342 | 21.862 | 1.00 | 50.00 | C |
| ATOM | 280 | CG1 | ILE | A | 64 | −6.450 | −0.336 | 21.009 | 1.00 | 50.00 | C |
| ATOM | 281 | CG2 | ILE | A | 64 | −5.591 | −2.731 | 21.229 | 1.00 | 50.00 | C |
| ATOM | 282 | CD1 | ILE | A | 64 | −5.874 | 0.006 | 19.636 | 1.00 | 50.00 | C |
| ATOM | 283 | N | LYS | A | 65 | −6.271 | −3.459 | 24.659 | 1.00 | 50.00 | N |
| ATOM | 284 | CA | LYS | A | 65 | −5.989 | −4.484 | 25.665 | 1.00 | 50.00 | C |
| ATOM | 285 | C | LYS | A | 65 | −4.815 | −5.440 | 25.489 | 1.00 | 50.00 | C |
| ATOM | 286 | O | LYS | A | 65 | −4.302 | −5.926 | 26.496 | 1.00 | 50.00 | O |
| ATOM | 287 | CB | LYS | A | 65 | −7.233 | −5.347 | 25.882 | 1.00 | 50.00 | C |
| ATOM | 288 | CG | LYS | A | 65 | −8.509 | −4.594 | 26.253 | 1.00 | 50.00 | C |
| ATOM | 289 | CD | LYS | A | 65 | −9.742 | −5.483 | 26.399 | 1.00 | 50.00 | C |
| ATOM | 290 | CE | LYS | A | 65 | −10.926 | −4.647 | 26.880 | 1.00 | 50.00 | C |
| ATOM | 291 | NZ | LYS | A | 65 | −12.106 | −5.481 | 27.152 | 1.00 | 50.00 | N |
| ATOM | 292 | N | LYS | A | 66 | −4.417 | −5.760 | 24.269 | 1.00 | 50.00 | N |
| ATOM | 293 | CA | LYS | A | 66 | −3.314 | −6.678 | 23.980 | 1.00 | 50.00 | C |
| ATOM | 294 | C | LYS | A | 66 | −3.462 | −8.164 | 24.290 | 1.00 | 50.00 | C |
| ATOM | 295 | O | LYS | A | 66 | −2.485 | −8.900 | 24.427 | 1.00 | 50.00 | O |
| ATOM | 296 | CB | LYS | A | 66 | −1.951 | −6.234 | 24.507 | 1.00 | 50.00 | C |
| ATOM | 297 | CG | LYS | A | 66 | −1.440 | −4.851 | 24.115 | 1.00 | 50.00 | C |
| ATOM | 298 | CD | LYS | A | 66 | −0.203 | −4.598 | 24.973 | 1.00 | 50.00 | C |
| ATOM | 299 | CE | LYS | A | 66 | 0.339 | −3.182 | 24.809 | 1.00 | 50.00 | C |
| ATOM | 300 | NZ | LYS | A | 66 | 1.736 | −3.106 | 25.256 | 1.00 | 50.00 | N |
| ATOM | 301 | N | ASN | A | 67 | −4.681 | −8.668 | 24.362 | 1.00 | 50.00 | N |
| ATOM | 302 | CA | ASN | A | 67 | −4.964 | −10.027 | 24.820 | 1.00 | 50.00 | C |
| ATOM | 303 | C | ASN | A | 67 | −4.666 | −11.037 | 23.717 | 1.00 | 50.00 | C |
| ATOM | 304 | O | ASN | A | 67 | −5.138 | −10.861 | 22.594 | 1.00 | 50.00 | O |
| ATOM | 305 | CB | ASN | A | 67 | −6.380 | −9.964 | 25.394 | 1.00 | 50.00 | C |
| ATOM | 306 | CG | ASN | A | 67 | −7.539 | −9.893 | 24.411 | 1.00 | 50.00 | C |
| ATOM | 307 | OD1 | ASN | A | 67 | −8.180 | −10.918 | 24.184 | 1.00 | 50.00 | O |
| ATOM | 308 | ND2 | ASN | A | 67 | −7.797 | −8.756 | 23.789 | 1.00 | 50.00 | N |
| ATOM | 309 | N | LYS | A | 68 | −3.842 | −12.032 | 24.008 | 1.00 | 50.00 | N |
| ATOM | 310 | CA | LYS | A | 68 | −3.178 | −12.902 | 23.034 | 1.00 | 50.00 | C |
| ATOM | 311 | C | LYS | A | 68 | −1.847 | −12.306 | 22.585 | 1.00 | 50.00 | C |
| ATOM | 312 | O | LYS | A | 68 | −1.451 | −11.220 | 23.009 | 1.00 | 50.00 | O |
| ATOM | 313 | CB | LYS | A | 68 | −4.078 | −13.489 | 21.942 | 1.00 | 50.00 | C |
| ATOM | 314 | CG | LYS | A | 68 | −5.317 | −14.213 | 22.466 | 1.00 | 50.00 | C |
| ATOM | 315 | CD | LYS | A | 68 | −6.307 | −14.730 | 21.424 | 1.00 | 50.00 | C |
| ATOM | 316 | CE | LYS | A | 68 | −7.469 | −15.432 | 22.123 | 1.00 | 50.00 | C |
| ATOM | 317 | NZ | LYS | A | 68 | −8.369 | −16.119 | 21.186 | 1.00 | 50.00 | N |
| ATOM | 318 | N | CYS | A | 69 | −1.055 | −12.966 | 21.756 | 1.00 | 50.00 | N |
| ATOM | 319 | CA | CYS | A | 69 | −0.700 | −12.485 | 20.419 | 1.00 | 50.00 | C |
| ATOM | 320 | C | CYS | A | 69 | −0.296 | −11.022 | 20.275 | 1.00 | 50.00 | C |
| ATOM | 321 | O | CYS | A | 69 | 0.879 | −10.734 | 20.050 | 1.00 | 50.00 | O |
| ATOM | 322 | CB | CYS | A | 69 | −1.725 | −12.990 | 19.403 | 1.00 | 50.00 | C |
| ATOM | 323 | SG | CYS | A | 69 | −1.735 | −14.786 | 19.523 | 1.00 | 50.00 | S |
| ATOM | 324 | N | ASN | A | 70 | −1.278 | −10.137 | 20.351 | 1.00 | 50.00 | N |
| ATOM | 325 | CA | ASN | A | 70 | −1.166 | −8.680 | 20.268 | 1.00 | 50.00 | C |
| ATOM | 326 | C | ASN | A | 70 | 0.080 | −7.963 | 20.776 | 1.00 | 50.00 | C |
| ATOM | 327 | O | ASN | A | 70 | 0.481 | −6.984 | 20.147 | 1.00 | 50.00 | O |
| ATOM | 328 | CB | ASN | A | 70 | −2.308 | −8.118 | 21.115 | 1.00 | 50.00 | C |
| ATOM | 329 | CG | ASN | A | 70 | −3.731 | −7.970 | 20.603 | 1.00 | 50.00 | C |
| ATOM | 330 | OD1 | ASN | A | 70 | −4.536 | −8.900 | 20.582 | 1.00 | 50.00 | O |
| ATOM | 331 | ND2 | ASN | A | 70 | −4.081 | −6.731 | 20.312 | 1.00 | 50.00 | N |
| ATOM | 332 | N | GLY | A | 71 | 0.572 | −8.367 | 21.937 | 1.00 | 50.00 | N |
| ATOM | 333 | CA | GLY | A | 71 | 1.712 | −7.771 | 22.633 | 1.00 | 50.00 | C |
| ATOM | 334 | C | GLY | A | 71 | 2.842 | −7.208 | 21.785 | 1.00 | 50.00 | C |
| ATOM | 335 | O | GLY | A | 71 | 3.143 | −6.017 | 21.839 | 1.00 | 50.00 | O |
| ATOM | 336 | N | THR | A | 72 | 3.321 | −8.080 | 20.917 | 1.00 | 50.00 | N |
| ATOM | 337 | CA | THR | A | 72 | 4.154 | −7.804 | 19.749 | 1.00 | 50.00 | C |
| ATOM | 338 | C | THR | A | 72 | 4.090 | −6.426 | 19.093 | 1.00 | 50.00 | C |
| ATOM | 339 | O | THR | A | 72 | 5.072 | −5.686 | 19.124 | 1.00 | 50.00 | O |
| ATOM | 340 | CB | THR | A | 72 | 3.784 | −9.025 | 18.902 | 1.00 | 50.00 | C |
| ATOM | 341 | OG1 | THR | A | 72 | 3.996 | −10.295 | 19.502 | 1.00 | 50.00 | O |
| ATOM | 342 | CG2 | THR | A | 72 | 4.652 | −9.068 | 17.653 | 1.00 | 50.00 | C |
| ATOM | 343 | N | ASP | A | 73 | 2.984 | −6.065 | 18.466 | 1.00 | 50.00 | N |
| ATOM | 344 | CA | ASP | A | 73 | 2.882 | −4.862 | 17.639 | 1.00 | 50.00 | C |
| ATOM | 345 | C | ASP | A | 73 | 2.529 | −3.600 | 18.408 | 1.00 | 50.00 | C |
| ATOM | 346 | O | ASP | A | 73 | 3.258 | −2.617 | 18.303 | 1.00 | 50.00 | O |
| ATOM | 347 | CB | ASP | A | 73 | 1.825 | −5.091 | 16.564 | 1.00 | 50.00 | C |
| ATOM | 348 | CG | ASP | A | 73 | 2.416 | −6.073 | 15.567 | 1.00 | 50.00 | C |
| ATOM | 349 | OD1 | ASP | A | 73 | 3.163 | −5.633 | 14.668 | 1.00 | 50.00 | O |
| ATOM | 350 | OD2 | ASP | A | 73 | 2.122 | −7.284 | 15.673 | 1.00 | 50.00 | O |
| ATOM | 351 | N | ALA | A | 74 | 1.532 | −3.651 | 19.272 | 1.00 | 50.00 | N |
| ATOM | 352 | CA | ALA | A | 74 | 1.481 | −2.812 | 20.467 | 1.00 | 50.00 | C |
| ATOM | 353 | C | ALA | A | 74 | 2.785 | −2.404 | 21.145 | 1.00 | 50.00 | C |
| ATOM | 354 | O | ALA | A | 74 | 2.969 | −1.206 | 21.354 | 1.00 | 50.00 | O |
| ATOM | 355 | CB | ALA | A | 74 | 0.539 | −3.603 | 21.370 | 1.00 | 50.00 | C |
| ATOM | 356 | N | LYS | A | 75 | 3.763 | −3.273 | 21.347 | 1.00 | 50.00 | N |
| ATOM | 357 | CA | LYS | A | 75 | 5.133 | −2.889 | 21.693 | 1.00 | 50.00 | C |
| ATOM | 358 | C | LYS | A | 75 | 5.743 | −1.870 | 20.740 | 1.00 | 50.00 | C |
| ATOM | 359 | O | LYS | A | 75 | 5.884 | −0.733 | 21.188 | 1.00 | 50.00 | O |
| ATOM | 360 | CB | LYS | A | 75 | 6.114 | −4.047 | 21.875 | 1.00 | 50.00 | C |
| ATOM | 361 | CG | LYS | A | 75 | 5.854 | −4.903 | 23.111 | 1.00 | 50.00 | C |
| ATOM | 362 | CD | LYS | A | 75 | 6.731 | −6.153 | 23.112 | 1.00 | 50.00 | C |
| ATOM | 363 | CE | LYS | A | 75 | 6.255 | −7.148 | 24.168 | 1.00 | 50.00 | C |
| ATOM | 364 | NZ | LYS | A | 75 | 7.222 | −8.249 | 24.289 | 1.00 | 50.00 | N |
| ATOM | 365 | N | VAL | A | 76 | 5.966 | −2.179 | 19.473 | 1.00 | 50.00 | N |
| ATOM | 366 | CA | VAL | A | 76 | 6.242 | −1.214 | 18.406 | 1.00 | 50.00 | C |
| ATOM | 367 | C | VAL | A | 76 | 5.578 | 0.157 | 18.501 | 1.00 | 50.00 | C |
| ATOM | 368 | O | VAL | A | 76 | 6.211 | 1.191 | 18.293 | 1.00 | 50.00 | O |
| ATOM | 369 | CB | VAL | A | 76 | 6.040 | −1.945 | 17.077 | 1.00 | 50.00 | C |
| ATOM | 370 | CG1 | VAL | A | 76 | 6.203 | −0.998 | 15.893 | 1.00 | 50.00 | C |
| ATOM | 371 | CG2 | VAL | A | 76 | 6.945 | −3.149 | 16.816 | 1.00 | 50.00 | C |
| ATOM | 372 | N | LYS | A | 77 | 4.297 | 0.174 | 18.821 | 1.00 | 50.00 | N |
| ATOM | 373 | CA | LYS | A | 77 | 3.435 | 1.354 | 18.882 | 1.00 | 50.00 | C |
| ATOM | 374 | C | LYS | A | 77 | 3.708 | 2.233 | 20.091 | 1.00 | 50.00 | C |
| ATOM | 375 | O | LYS | A | 77 | 4.216 | 3.340 | 19.927 | 1.00 | 50.00 | O |
| ATOM | 376 | CB | LYS | A | 77 | 1.985 | 0.873 | 18.876 | 1.00 | 50.00 | C |
| ATOM | 377 | CG | LYS | A | 77 | 1.783 | 0.090 | 17.585 | 1.00 | 50.00 | C |
| ATOM | 378 | CD | LYS | A | 77 | 0.345 | −0.335 | 17.341 | 1.00 | 50.00 | C |
| ATOM | 379 | CE | LYS | A | 77 | 0.439 | −1.046 | 15.996 | 1.00 | 50.00 | C |
| ATOM | 380 | NZ | LYS | A | 77 | −0.813 | −1.191 | 15.254 | 1.00 | 50.00 | N |
| ATOM | 381 | N | LEU | A | 78 | 3.450 | 1.687 | 21.266 | 1.00 | 50.00 | N |
| ATOM | 382 | CA | LEU | A | 78 | 3.853 | 2.222 | 22.565 | 1.00 | 50.00 | C |
| ATOM | 383 | C | LEU | A | 78 | 5.335 | 2.524 | 22.770 | 1.00 | 50.00 | C |
| ATOM | 384 | O | LEU | A | 78 | 5.653 | 3.345 | 23.630 | 1.00 | 50.00 | O |
| ATOM | 385 | CB | LEU | A | 78 | 3.352 | 1.196 | 23.581 | 1.00 | 50.00 | C |
| ATOM | 386 | CG | LEU | A | 78 | 1.849 | 1.049 | 23.831 | 1.00 | 50.00 | C |
| ATOM | 387 | CD1 | LEU | A | 78 | 0.942 | 0.479 | 22.740 | 1.00 | 50.00 | C |
| ATOM | 388 | CD2 | LEU | A | 78 | 1.705 | 0.156 | 25.060 | 1.00 | 50.00 | C |
| ATOM | 389 | N | ILE | A | 79 | 6.225 | 1.968 | 21.962 | 1.00 | 50.00 | N |
| ATOM | 390 | CA | ILE | A | 79 | 7.565 | 2.487 | 21.689 | 1.00 | 50.00 | C |
| ATOM | 391 | C | ILE | A | 79 | 7.357 | 3.856 | 21.050 | 1.00 | 50.00 | C |
| ATOM | 392 | O | ILE | A | 79 | 7.469 | 4.850 | 21.764 | 1.00 | 50.00 | O |
| ATOM | 393 | CB | ILE | A | 79 | 8.448 | 1.485 | 20.937 | 1.00 | 50.00 | C |
| ATOM | 394 | CG1 | ILE | A | 79 | 8.730 | 0.192 | 21.705 | 1.00 | 50.00 | C |
| ATOM | 395 | CG2 | ILE | A | 79 | 9.796 | 2.048 | 20.485 | 1.00 | 50.00 | C |
| ATOM | 396 | CD1 | ILE | A | 79 | 9.223 | −0.994 | 20.877 | 1.00 | 50.00 | C |
| ATOM | 397 | N | LYS | A | 80 | 7.011 | 3.954 | 19.778 | 1.00 | 50.00 | N |
| ATOM | 398 | CA | LYS | A | 80 | 6.947 | 5.190 | 18.995 | 1.00 | 50.00 | C |
| ATOM | 399 | C | LYS | A | 80 | 6.088 | 6.362 | 19.449 | 1.00 | 50.00 | C |
| ATOM | 400 | O | LYS | A | 80 | 6.478 | 7.512 | 19.258 | 1.00 | 50.00 | O |
| ATOM | 401 | CB | LYS | A | 80 | 6.444 | 4.818 | 17.604 | 1.00 | 50.00 | C |
| ATOM | 402 | CG | LYS | A | 80 | 7.390 | 3.807 | 16.960 | 1.00 | 50.00 | C |
| ATOM | 403 | CD | LYS | A | 80 | 6.705 | 3.344 | 15.683 | 1.00 | 50.00 | C |
| ATOM | 404 | CE | LYS | A | 80 | 7.479 | 2.247 | 14.964 | 1.00 | 50.00 | C |
| ATOM | 405 | NZ | LYS | A | 80 | 7.353 | 2.335 | 13.504 | 1.00 | 50.00 | N |
| ATOM | 406 | N | GLN | A | 81 | 4.944 | 6.073 | 20.045 | 1.00 | 50.00 | N |
| ATOM | 407 | CA | GLN | A | 81 | 4.220 | 6.944 | 20.970 | 1.00 | 50.00 | C |
| ATOM | 408 | C | GLN | A | 81 | 5.078 | 7.727 | 21.958 | 1.00 | 50.00 | C |
| ATOM | 409 | O | GLN | A | 81 | 4.952 | 8.948 | 22.040 | 1.00 | 50.00 | O |
| ATOM | 410 | CB | GLN | A | 81 | 3.209 | 6.054 | 21.696 | 1.00 | 50.00 | C |
| ATOM | 411 | CG | GLN | A | 81 | 2.081 | 5.442 | 20.866 | 1.00 | 50.00 | C |
| ATOM | 412 | CD | GLN | A | 81 | 1.029 | 6.400 | 20.329 | 1.00 | 50.00 | C |
| ATOM | 413 | OE1 | GLN | A | 81 | 1.043 | 7.612 | 20.541 | 1.00 | 50.00 | O |
| ATOM | 414 | NE2 | GLN | A | 81 | 0.101 | 5.855 | 19.564 | 1.00 | 50.00 | N |
| ATOM | 415 | N | GLU | A | 82 | 5.982 | 7.046 | 22.642 | 1.00 | 50.00 | N |
| ATOM | 416 | CA | GLU | A | 82 | 7.070 | 7.633 | 23.424 | 1.00 | 50.00 | C |
| ATOM | 417 | C | GLU | A | 82 | 8.244 | 8.290 | 22.711 | 1.00 | 50.00 | C |
| ATOM | 418 | O | GLU | A | 82 | 8.765 | 9.278 | 23.228 | 1.00 | 50.00 | O |
| ATOM | 419 | CB | GLU | A | 82 | 7.554 | 6.587 | 24.431 | 1.00 | 50.00 | C |
| ATOM | 420 | CG | GLU | A | 82 | 6.565 | 6.326 | 25.566 | 1.00 | 50.00 | C |
| ATOM | 421 | CD | GLU | A | 82 | 6.363 | 7.640 | 26.308 | 1.00 | 50.00 | C |
| ATOM | 422 | OE1 | GLU | A | 82 | 5.263 | 8.228 | 26.231 | 1.00 | 50.00 | O |
| ATOM | 423 | OE2 | GLU | A | 82 | 7.364 | 8.199 | 26.805 | 1.00 | 50.00 | O |
| ATOM | 424 | N | LEU | A | 83 | 8.659 | 7.812 | 21.551 | 1.00 | 50.00 | N |
| ATOM | 425 | CA | LEU | A | 83 | 9.630 | 8.493 | 20.691 | 1.00 | 50.00 | C |
| ATOM | 426 | C | LEU | A | 83 | 9.118 | 9.814 | 20.123 | 1.00 | 50.00 | C |
| ATOM | 427 | O | LEU | A | 83 | 9.879 | 10.779 | 20.072 | 1.00 | 50.00 | O |
| ATOM | 428 | CB | LEU | A | 83 | 10.122 | 7.523 | 19.615 | 1.00 | 50.00 | C |
| ATOM | 429 | CG | LEU | A | 83 | 10.985 | 6.314 | 19.987 | 1.00 | 50.00 | C |
| ATOM | 430 | CD1 | LEU | A | 83 | 10.425 | 5.388 | 21.064 | 1.00 | 50.00 | C |
| ATOM | 431 | CD2 | LEU | A | 83 | 11.216 | 5.422 | 18.768 | 1.00 | 50.00 | C |
| ATOM | 432 | N | ASP | A | 84 | 7.843 | 9.900 | 19.773 | 1.00 | 50.00 | N |
| ATOM | 433 | CA | ASP | A | 84 | 7.086 | 11.129 | 19.538 | 1.00 | 50.00 | C |
| ATOM | 434 | C | ASP | A | 84 | 7.017 | 12.030 | 20.766 | 1.00 | 50.00 | C |
| ATOM | 435 | O | ASP | A | 84 | 7.655 | 13.082 | 20.804 | 1.00 | 50.00 | O |
| ATOM | 436 | CB | ASP | A | 84 | 5.671 | 10.805 | 19.050 | 1.00 | 50.00 | C |
| ATOM | 437 | CG | ASP | A | 84 | 5.451 | 10.265 | 17.647 | 1.00 | 50.00 | C |
| ATOM | 438 | OD1 | ASP | A | 84 | 5.816 | 10.964 | 16.678 | 1.00 | 50.00 | O |
| ATOM | 439 | OD2 | ASP | A | 84 | 4.870 | 9.166 | 17.508 | 1.00 | 50.00 | O |
| ATOM | 440 | N | LYS | A | 85 | 6.248 | 11.638 | 21.768 | 1.00 | 50.00 | N |
| ATOM | 441 | CA | LYS | A | 85 | 5.797 | 12.519 | 22.844 | 1.00 | 50.00 | C |
| ATOM | 442 | C | LYS | A | 85 | 6.767 | 12.866 | 23.962 | 1.00 | 50.00 | C |
| ATOM | 443 | O | LYS | A | 85 | 6.479 | 13.852 | 24.639 | 1.00 | 50.00 | O |
| ATOM | 444 | CB | LYS | A | 85 | 4.594 | 11.889 | 23.543 | 1.00 | 50.00 | C |
| ATOM | 445 | CG | LYS | A | 85 | 3.370 | 11.954 | 22.636 | 1.00 | 50.00 | C |
| ATOM | 446 | CD | LYS | A | 85 | 2.143 | 11.549 | 23.443 | 1.00 | 50.00 | C |
| ATOM | 447 | CE | LYS | A | 85 | 0.934 | 11.787 | 22.544 | 1.00 | 50.00 | C |
| ATOM | 448 | NZ | LYS | A | 85 | −0.178 | 12.005 | 23.477 | 1.00 | 50.00 | N |
| ATOM | 449 | N | TYR | A | 86 | 7.838 | 12.120 | 24.177 | 1.00 | 50.00 | N |
| ATOM | 450 | CA | TYR | A | 86 | 8.871 | 12.453 | 25.158 | 1.00 | 50.00 | C |
| ATOM | 451 | C | TYR | A | 86 | 9.248 | 13.926 | 25.272 | 1.00 | 50.00 | C |
| ATOM | 452 | O | TYR | A | 86 | 9.328 | 14.456 | 26.378 | 1.00 | 50.00 | O |
| ATOM | 453 | CB | TYR | A | 86 | 10.090 | 11.560 | 24.919 | 1.00 | 50.00 | C |
| ATOM | 454 | CG | TYR | A | 86 | 11.212 | 11.756 | 25.917 | 1.00 | 50.00 | C |
| ATOM | 455 | CD1 | TYR | A | 86 | 11.064 | 11.293 | 27.175 | 1.00 | 50.00 | C |
| ATOM | 456 | CD2 | TYR | A | 86 | 12.214 | 12.613 | 25.631 | 1.00 | 50.00 | C |
| ATOM | 457 | CE1 | TYR | A | 86 | 11.910 | 11.687 | 28.148 | 1.00 | 50.00 | C |
| ATOM | 458 | CE2 | TYR | A | 86 | 13.046 | 13.022 | 26.609 | 1.00 | 50.00 | C |
| ATOM | 459 | CZ | TYR | A | 86 | 12.907 | 12.550 | 27.863 | 1.00 | 50.00 | C |
| ATOM | 460 | OH | TYR | A | 86 | 13.725 | 13.031 | 28.851 | 1.00 | 50.00 | O |
| ATOM | 461 | N | LYS | A | 87 | 9.484 | 14.577 | 24.147 | 1.00 | 50.00 | N |
| ATOM | 462 | CA | LYS | A | 87 | 9.923 | 15.970 | 24.064 | 1.00 | 50.00 | C |
| ATOM | 463 | C | LYS | A | 87 | 8.841 | 17.014 | 24.288 | 1.00 | 50.00 | C |
| ATOM | 464 | O | LYS | A | 87 | 9.094 | 17.992 | 24.988 | 1.00 | 50.00 | O |
| ATOM | 465 | CB | LYS | A | 87 | 10.422 | 16.213 | 22.643 | 1.00 | 50.00 | C |
| ATOM | 466 | CG | LYS | A | 87 | 11.551 | 15.244 | 22.309 | 1.00 | 50.00 | C |
| ATOM | 467 | CD | LYS | A | 87 | 11.734 | 15.327 | 20.802 | 1.00 | 50.00 | C |
| ATOM | 468 | CE | LYS | A | 87 | 12.887 | 14.436 | 20.353 | 1.00 | 50.00 | C |
| ATOM | 469 | NZ | LYS | A | 87 | 13.611 | 15.274 | 19.393 | 1.00 | 50.00 | N |
| ATOM | 470 | N | ASN | A | 88 | 7.665 | 16.809 | 23.719 | 1.00 | 50.00 | N |
| ATOM | 471 | CA | ASN | A | 88 | 6.434 | 17.471 | 24.151 | 1.00 | 50.00 | C |
| ATOM | 472 | C | ASN | A | 88 | 6.181 | 17.373 | 25.647 | 1.00 | 50.00 | C |
| ATOM | 473 | O | ASN | A | 88 | 6.076 | 18.437 | 26.254 | 1.00 | 50.00 | O |
| ATOM | 474 | CB | ASN | A | 88 | 5.216 | 16.959 | 23.384 | 1.00 | 50.00 | C |
| ATOM | 475 | CG | ASN | A | 88 | 5.324 | 17.250 | 21.897 | 1.00 | 50.00 | C |
| ATOM | 476 | OD1 | ASN | A | 88 | 5.540 | 16.324 | 21.117 | 1.00 | 50.00 | O |
| ATOM | 477 | ND2 | ASN | A | 88 | 5.309 | 18.525 | 21.551 | 1.00 | 50.00 | N |
| ATOM | 478 | N | ALA | A | 89 | 6.273 | 16.196 | 26.245 | 1.00 | 50.00 | N |
| ATOM | 479 | CA | ALA | A | 89 | 6.455 | 16.034 | 27.686 | 1.00 | 50.00 | C |
| ATOM | 480 | C | ALA | A | 89 | 7.568 | 16.917 | 28.239 | 1.00 | 50.00 | C |
| ATOM | 481 | O | ALA | A | 89 | 7.191 | 17.964 | 28.762 | 1.00 | 50.00 | O |
| ATOM | 482 | CB | ALA | A | 89 | 6.520 | 14.549 | 28.042 | 1.00 | 50.00 | C |
| ATOM | 483 | N | VAL | A | 90 | 8.857 | 16.693 | 28.034 | 1.00 | 50.00 | N |
| ATOM | 484 | CA | VAL | A | 90 | 9.956 | 17.554 | 28.480 | 1.00 | 50.00 | C |
| ATOM | 485 | C | VAL | A | 90 | 9.754 | 19.063 | 28.389 | 1.00 | 50.00 | C |
| ATOM | 486 | O | VAL | A | 90 | 9.923 | 19.729 | 29.410 | 1.00 | 50.00 | O |
| ATOM | 487 | CB | VAL | A | 90 | 11.301 | 17.169 | 27.856 | 1.00 | 50.00 | C |
| ATOM | 488 | CG1 | VAL | A | 90 | 12.485 | 18.084 | 28.172 | 1.00 | 50.00 | C |
| ATOM | 489 | CG2 | VAL | A | 90 | 11.762 | 15.759 | 28.221 | 1.00 | 50.00 | C |
| ATOM | 490 | N | THR | A | 91 | 9.431 | 19.603 | 27.224 | 1.00 | 50.00 | N |
| ATOM | 491 | CA | THR | A | 91 | 9.048 | 21.000 | 27.014 | 1.00 | 50.00 | C |
| ATOM | 492 | C | THR | A | 91 | 8.023 | 21.443 | 28.050 | 1.00 | 50.00 | C |
| ATOM | 493 | O | THR | A | 91 | 8.418 | 22.206 | 28.931 | 1.00 | 50.00 | O |
| ATOM | 494 | CB | THR | A | 91 | 8.636 | 21.269 | 25.565 | 1.00 | 50.00 | C |
| ATOM | 495 | OG1 | THR | A | 91 | 9.744 | 21.077 | 24.696 | 1.00 | 50.00 | O |
| ATOM | 496 | CG2 | THR | A | 91 | 8.113 | 22.676 | 25.279 | 1.00 | 50.00 | C |
| ATOM | 497 | N | GLU | A | 92 | 6.821 | 20.889 | 28.053 | 1.00 | 50.00 | N |
| ATOM | 498 | CA | GLU | A | 92 | 5.832 | 21.027 | 29.123 | 1.00 | 50.00 | C |
| ATOM | 499 | C | GLU | A | 92 | 6.374 | 21.232 | 30.533 | 1.00 | 50.00 | C |
| ATOM | 500 | O | GLU | A | 92 | 6.237 | 22.330 | 31.070 | 1.00 | 50.00 | O |
| ATOM | 501 | CB | GLU | A | 92 | 4.787 | 19.913 | 29.038 | 1.00 | 50.00 | C |
| ATOM | 502 | CG | GLU | A | 92 | 3.968 | 19.880 | 27.748 | 1.00 | 50.00 | C |
| ATOM | 503 | CD | GLU | A | 92 | 3.045 | 21.085 | 27.697 | 1.00 | 50.00 | C |
| ATOM | 504 | OE1 | GLU | A | 92 | 3.080 | 21.868 | 26.723 | 1.00 | 50.00 | O |
| ATOM | 505 | OE2 | GLU | A | 92 | 2.340 | 21.296 | 28.707 | 1.00 | 50.00 | O |
| ATOM | 506 | N | LEU | A | 93 | 7.172 | 20.306 | 31.038 | 1.00 | 50.00 | N |
| ATOM | 507 | CA | LEU | A | 93 | 7.884 | 20.415 | 32.312 | 1.00 | 50.00 | C |
| ATOM | 508 | C | LEU | A | 93 | 8.679 | 21.708 | 32.454 | 1.00 | 50.00 | C |
| ATOM | 509 | O | LEU | A | 93 | 8.244 | 22.544 | 33.245 | 1.00 | 50.00 | O |
| ATOM | 510 | CB | LEU | A | 93 | 8.695 | 19.143 | 32.565 | 1.00 | 50.00 | C |
| ATOM | 511 | CG | LEU | A | 93 | 7.985 | 17.809 | 32.809 | 1.00 | 50.00 | C |
| ATOM | 512 | CD1 | LEU | A | 93 | 7.068 | 17.315 | 31.692 | 1.00 | 50.00 | C |
| ATOM | 513 | CD2 | LEU | A | 93 | 9.023 | 16.704 | 32.997 | 1.00 | 50.00 | C |
| ATOM | 514 | N | GLN | A | 94 | 9.691 | 21.970 | 31.641 | 1.00 | 50.00 | N |
| ATOM | 515 | CA | GLN | A | 94 | 10.408 | 23.245 | 31.546 | 1.00 | 50.00 | C |
| ATOM | 516 | C | GLN | A | 94 | 9.640 | 24.563 | 31.590 | 1.00 | 50.00 | C |
| ATOM | 517 | O | GLN | A | 94 | 10.071 | 25.502 | 32.259 | 1.00 | 50.00 | O |
| ATOM | 518 | CB | GLN | A | 94 | 11.304 | 23.217 | 30.306 | 1.00 | 50.00 | C |
| ATOM | 519 | CG | GLN | A | 94 | 12.447 | 22.201 | 30.287 | 1.00 | 50.00 | C |
| ATOM | 520 | CD | GLN | A | 94 | 13.567 | 22.453 | 31.287 | 1.00 | 50.00 | C |
| ATOM | 521 | OE1 | GLN | A | 94 | 13.649 | 23.480 | 31.959 | 1.00 | 50.00 | O |
| ATOM | 522 | NE2 | GLN | A | 94 | 14.481 | 21.509 | 31.418 | 1.00 | 50.00 | N |
| ATOM | 523 | N | LEU | A | 95 | 8.514 | 24.642 | 30.902 | 1.00 | 50.00 | N |
| ATOM | 524 | CA | LEU | A | 95 | 7.598 | 25.784 | 30.893 | 1.00 | 50.00 | C |
| ATOM | 525 | C | LEU | A | 95 | 6.805 | 25.952 | 32.186 | 1.00 | 50.00 | C |
| ATOM | 526 | O | LEU | A | 95 | 6.754 | 27.052 | 32.734 | 1.00 | 50.00 | O |
| ATOM | 527 | CB | LEU | A | 95 | 6.663 | 25.616 | 29.694 | 1.00 | 50.00 | C |
| ATOM | 528 | CG | LEU | A | 95 | 7.183 | 25.817 | 28.270 | 1.00 | 50.00 | C |
| ATOM | 529 | CD1 | LEU | A | 95 | 8.446 | 25.089 | 27.816 | 1.00 | 50.00 | C |
| ATOM | 530 | CD2 | LEU | A | 95 | 6.082 | 25.429 | 27.283 | 1.00 | 50.00 | C |
| ATOM | 531 | N | LEU | A | 96 | 6.196 | 24.883 | 32.673 | 1.00 | 50.00 | N |
| ATOM | 532 | CA | LEU | A | 96 | 5.479 | 24.796 | 33.947 | 1.00 | 50.00 | C |
| ATOM | 533 | C | LEU | A | 96 | 6.304 | 25.026 | 35.210 | 1.00 | 50.00 | C |
| ATOM | 534 | O | LEU | A | 96 | 5.762 | 25.576 | 36.169 | 1.00 | 50.00 | O |
| ATOM | 535 | CB | LEU | A | 96 | 4.781 | 23.435 | 33.985 | 1.00 | 50.00 | C |
| ATOM | 536 | CG | LEU | A | 96 | 3.503 | 23.197 | 33.176 | 1.00 | 50.00 | C |
| ATOM | 537 | CD1 | LEU | A | 96 | 3.532 | 23.395 | 31.661 | 1.00 | 50.00 | C |
| ATOM | 538 | CD2 | LEU | A | 96 | 3.001 | 21.777 | 33.437 | 1.00 | 50.00 | C |
| ATOM | 539 | N | MET | A | 97 | 7.576 | 24.661 | 35.241 | 1.00 | 50.00 | N |
| ATOM | 540 | CA | MET | A | 97 | 8.551 | 25.060 | 36.258 | 1.00 | 50.00 | C |
| ATOM | 541 | C | MET | A | 97 | 8.661 | 26.547 | 36.578 | 1.00 | 50.00 | C |
| ATOM | 542 | O | MET | A | 97 | 8.809 | 26.926 | 37.739 | 1.00 | 50.00 | O |
| ATOM | 543 | CB | MET | A | 97 | 9.926 | 24.516 | 35.866 | 1.00 | 50.00 | C |
| ATOM | 544 | CG | MET | A | 97 | 10.094 | 22.998 | 35.894 | 1.00 | 50.00 | C |
| ATOM | 545 | SD | MET | A | 97 | 9.680 | 22.307 | 37.504 | 1.00 | 50.00 | S |
| ATOM | 546 | CE | MET | A | 97 | 11.165 | 22.741 | 38.425 | 1.00 | 50.00 | C |
| ATOM | 547 | N | GLN | A | 98 | 8.577 | 27.388 | 35.562 | 1.00 | 50.00 | N |
| ATOM | 548 | CA | GLN | A | 98 | 8.565 | 28.845 | 35.671 | 1.00 | 50.00 | C |
| ATOM | 549 | C | GLN | A | 98 | 7.186 | 29.426 | 35.955 | 1.00 | 50.00 | C |
| ATOM | 550 | O | GLN | A | 98 | 6.140 | 28.781 | 36.023 | 1.00 | 50.00 | O |
| ATOM | 551 | CB | GLN | A | 98 | 9.018 | 29.328 | 34.295 | 1.00 | 50.00 | C |
| ATOM | 552 | CG | GLN | A | 98 | 10.460 | 29.091 | 33.844 | 1.00 | 50.00 | C |
| ATOM | 553 | CD | GLN | A | 98 | 11.508 | 29.943 | 34.544 | 1.00 | 50.00 | C |
| ATOM | 554 | OE1 | GLN | A | 98 | 11.223 | 30.852 | 35.322 | 1.00 | 50.00 | O |
| ATOM | 555 | NE2 | GLN | A | 98 | 12.770 | 29.675 | 34.266 | 1.00 | 50.00 | N |
| ATOM | 556 | N | LEU | B | 160 | 5.047 | −92.812 | 3.477 | 1.00 | 50.00 | N |
| ATOM | 557 | CA | LEU | B | 160 | 3.680 | −92.660 | 3.997 | 1.00 | 50.00 | C |
| ATOM | 558 | C | LEU | B | 160 | 3.483 | −92.182 | 5.428 | 1.00 | 50.00 | C |
| ATOM | 559 | O | LEU | B | 160 | 2.770 | −91.213 | 5.682 | 1.00 | 50.00 | O |
| ATOM | 560 | CB | LEU | B | 160 | 2.616 | −93.650 | 3.530 | 1.00 | 50.00 | C |
| ATOM | 561 | CG | LEU | B | 160 | 2.761 | −95.169 | 3.454 | 1.00 | 50.00 | C |
| ATOM | 562 | CD1 | LEU | B | 160 | 3.973 | −95.770 | 4.163 | 1.00 | 50.00 | C |
| ATOM | 563 | CD2 | LEU | B | 160 | 1.509 | −95.789 | 4.065 | 1.00 | 50.00 | C |
| ATOM | 564 | N | GLU | B | 161 | 4.040 | −92.892 | 6.392 | 1.00 | 50.00 | N |
| ATOM | 565 | CA | GLU | B | 161 | 4.382 | −92.332 | 7.700 | 1.00 | 50.00 | C |
| ATOM | 566 | C | GLU | B | 161 | 5.506 | −91.302 | 7.650 | 1.00 | 50.00 | C |
| ATOM | 567 | O | GLU | B | 161 | 5.396 | −90.250 | 8.279 | 1.00 | 50.00 | O |
| ATOM | 568 | CB | GLU | B | 161 | 4.724 | −93.510 | 8.613 | 1.00 | 50.00 | C |
| ATOM | 569 | CG | GLU | B | 161 | 5.296 | −93.187 | 9.993 | 1.00 | 50.00 | C |
| ATOM | 570 | CD | GLU | B | 161 | 4.405 | −92.337 | 10.887 | 1.00 | 50.00 | C |
| ATOM | 571 | OE1 | GLU | B | 161 | 3.461 | −92.882 | 11.496 | 1.00 | 50.00 | O |
| ATOM | 572 | OE2 | GLU | B | 161 | 4.642 | −91.113 | 10.986 | 1.00 | 50.00 | O |
| ATOM | 573 | N | GLY | B | 162 | 6.596 | −91.609 | 6.965 | 1.00 | 50.00 | N |
| ATOM | 574 | CA | GLY | B | 162 | 7.701 | −90.681 | 6.727 | 1.00 | 50.00 | C |
| ATOM | 575 | C | GLY | B | 162 | 7.440 | −89.363 | 6.012 | 1.00 | 50.00 | C |
| ATOM | 576 | O | GLY | B | 162 | 8.189 | −88.410 | 6.217 | 1.00 | 50.00 | O |
| ATOM | 577 | N | GLU | B | 163 | 6.379 | −89.292 | 5.226 | 1.00 | 50.00 | N |
| ATOM | 578 | CA | GLU | B | 163 | 5.779 | −88.024 | 4.818 | 1.00 | 50.00 | C |
| ATOM | 579 | C | GLU | B | 163 | 4.957 | −87.404 | 5.948 | 1.00 | 50.00 | C |
| ATOM | 580 | O | GLU | B | 163 | 5.029 | −86.178 | 5.961 | 1.00 | 50.00 | O |
| ATOM | 581 | CB | GLU | B | 163 | 5.115 | −88.239 | 3.457 | 1.00 | 50.00 | C |
| ATOM | 582 | CG | GLU | B | 163 | 5.734 | −88.955 | 2.257 | 1.00 | 50.00 | C |
| ATOM | 583 | CD | GLU | B | 163 | 6.820 | −88.182 | 1.530 | 1.00 | 50.00 | C |
| ATOM | 584 | OE1 | GLU | B | 163 | 7.994 | −88.382 | 1.907 | 1.00 | 50.00 | O |
| ATOM | 585 | OE2 | GLU | B | 163 | 6.532 | −87.418 | 0.583 | 1.00 | 50.00 | O |
| ATOM | 586 | N | VAL | B | 164 | 4.230 | −88.034 | 6.863 | 1.00 | 50.00 | N |
| ATOM | 587 | CA | VAL | B | 164 | 3.231 | −87.439 | 7.762 | 1.00 | 50.00 | C |
| ATOM | 588 | C | VAL | B | 164 | 3.684 | −86.438 | 8.820 | 1.00 | 50.00 | C |
| ATOM | 589 | O | VAL | B | 164 | 2.968 | −85.532 | 9.244 | 1.00 | 50.00 | O |
| ATOM | 590 | CB | VAL | B | 164 | 2.112 | −88.403 | 8.170 | 1.00 | 50.00 | C |
| ATOM | 591 | CG1 | VAL | B | 164 | 2.373 | −89.263 | 9.405 | 1.00 | 50.00 | C |
| ATOM | 592 | CG2 | VAL | B | 164 | 0.719 | −87.774 | 8.231 | 1.00 | 50.00 | C |
| ATOM | 593 | N | ASN | B | 165 | 4.946 | −86.629 | 9.154 | 1.00 | 50.00 | N |
| ATOM | 594 | CA | ASN | B | 165 | 5.892 | −85.660 | 9.701 | 1.00 | 50.00 | C |
| ATOM | 595 | C | ASN | B | 165 | 6.454 | −84.558 | 8.803 | 1.00 | 50.00 | C |
| ATOM | 596 | O | ASN | B | 165 | 6.780 | −83.510 | 9.359 | 1.00 | 50.00 | O |
| ATOM | 597 | CB | ASN | B | 165 | 6.915 | −86.611 | 10.326 | 1.00 | 50.00 | C |
| ATOM | 598 | CG | ASN | B | 165 | 7.907 | −87.329 | 9.417 | 1.00 | 50.00 | C |
| ATOM | 599 | OD1 | ASN | B | 165 | 8.535 | −86.741 | 8.538 | 1.00 | 50.00 | O |
| ATOM | 600 | ND2 | ASN | B | 165 | 8.100 | −88.624 | 9.584 | 1.00 | 50.00 | N |
| ATOM | 601 | N | LYS | B | 166 | 6.635 | −84.706 | 7.498 | 1.00 | 50.00 | N |
| ATOM | 602 | CA | LYS | B | 166 | 7.224 | −83.732 | 6.573 | 1.00 | 50.00 | C |
| ATOM | 603 | C | LYS | B | 166 | 6.512 | −82.384 | 6.539 | 1.00 | 50.00 | C |
| ATOM | 604 | O | LYS | B | 166 | 7.027 | −81.354 | 6.967 | 1.00 | 50.00 | O |
| ATOM | 605 | CB | LYS | B | 166 | 7.393 | −84.232 | 5.135 | 1.00 | 50.00 | C |
| ATOM | 606 | CG | LYS | B | 166 | 8.519 | −85.229 | 4.875 | 1.00 | 50.00 | C |
| ATOM | 607 | CD | LYS | B | 166 | 8.852 | −85.450 | 3.400 | 1.00 | 50.00 | C |
| ATOM | 608 | CE | LYS | B | 166 | 9.978 | −86.471 | 3.252 | 1.00 | 50.00 | C |
| ATOM | 609 | NZ | LYS | B | 166 | 10.301 | −86.766 | 1.848 | 1.00 | 50.00 | N |
| ATOM | 610 | N | ILE | B | 167 | 5.322 | −82.385 | 5.968 | 1.00 | 50.00 | N |
| ATOM | 611 | CA | ILE | B | 167 | 4.166 | −81.556 | 6.308 | 1.00 | 50.00 | C |
| ATOM | 612 | C | ILE | B | 167 | 3.755 | −81.059 | 7.684 | 1.00 | 50.00 | C |
| ATOM | 613 | O | ILE | B | 167 | 3.099 | −80.024 | 7.785 | 1.00 | 50.00 | O |
| ATOM | 614 | CB | ILE | B | 167 | 2.938 | −82.381 | 5.923 | 1.00 | 50.00 | C |
| ATOM | 615 | CG1 | ILE | B | 167 | 2.945 | −83.812 | 6.468 | 1.00 | 50.00 | C |
| ATOM | 616 | CG2 | ILE | B | 167 | 2.825 | −82.148 | 4.420 | 1.00 | 50.00 | C |
| ATOM | 617 | CD1 | ILE | B | 167 | 2.858 | −84.789 | 5.301 | 1.00 | 50.00 | C |
| ATOM | 618 | N | LYS | B | 168 | 3.921 | −81.863 | 8.716 | 1.00 | 50.00 | N |
| ATOM | 619 | CA | LYS | B | 168 | 3.784 | −81.385 | 10.090 | 1.00 | 50.00 | C |
| ATOM | 620 | C | LYS | B | 168 | 4.895 | −80.393 | 10.426 | 1.00 | 50.00 | C |
| ATOM | 621 | O | LYS | B | 168 | 4.588 | −79.285 | 10.862 | 1.00 | 50.00 | O |
| ATOM | 622 | CB | LYS | B | 168 | 3.686 | −82.695 | 10.868 | 1.00 | 50.00 | C |
| ATOM | 623 | CG | LYS | B | 168 | 3.419 | −82.471 | 12.352 | 1.00 | 50.00 | C |
| ATOM | 624 | CD | LYS | B | 168 | 3.561 | −83.738 | 13.193 | 1.00 | 50.00 | C |
| ATOM | 625 | CE | LYS | B | 168 | 3.686 | −83.437 | 14.685 | 1.00 | 50.00 | C |
| ATOM | 626 | NZ | LYS | B | 168 | 4.904 | −82.674 | 15.001 | 1.00 | 50.00 | N |
| ATOM | 627 | N | SER | B | 169 | 6.140 | −80.727 | 10.122 | 1.00 | 50.00 | N |
| ATOM | 628 | CA | SER | B | 169 | 7.282 | −79.815 | 10.048 | 1.00 | 50.00 | C |
| ATOM | 629 | C | SER | B | 169 | 7.196 | −78.599 | 9.134 | 1.00 | 50.00 | C |
| ATOM | 630 | O | SER | B | 169 | 7.613 | −77.516 | 9.541 | 1.00 | 50.00 | O |
| ATOM | 631 | CB | SER | B | 169 | 8.534 | −80.613 | 9.686 | 1.00 | 50.00 | C |
| ATOM | 632 | OG | SER | B | 169 | 8.889 | −81.488 | 10.747 | 1.00 | 50.00 | O |
| ATOM | 633 | N | ALA | B | 170 | 6.681 | −78.735 | 7.923 | 1.00 | 50.00 | N |
| ATOM | 634 | CA | ALA | B | 170 | 6.380 | −77.599 | 7.053 | 1.00 | 50.00 | C |
| ATOM | 635 | C | ALA | B | 170 | 5.227 | −76.744 | 7.577 | 1.00 | 50.00 | C |
| ATOM | 636 | O | ALA | B | 170 | 5.352 | −75.526 | 7.457 | 1.00 | 50.00 | O |
| ATOM | 637 | CB | ALA | B | 170 | 6.461 | −78.178 | 5.640 | 1.00 | 50.00 | C |
| ATOM | 638 | N | LEU | B | 171 | 4.178 | −77.261 | 8.204 | 1.00 | 50.00 | N |
| ATOM | 639 | CA | LEU | B | 171 | 3.028 | −76.538 | 8.755 | 1.00 | 50.00 | C |
| ATOM | 640 | C | LEU | B | 171 | 3.342 | −75.629 | 9.931 | 1.00 | 50.00 | C |
| ATOM | 641 | O | LEU | B | 171 | 2.952 | −74.463 | 9.956 | 1.00 | 50.00 | O |
| ATOM | 642 | CB | LEU | B | 171 | 1.827 | −77.420 | 9.101 | 1.00 | 50.00 | C |
| ATOM | 643 | CG | LEU | B | 171 | 0.413 | −76.935 | 8.779 | 1.00 | 50.00 | C |
| ATOM | 644 | CD1 | LEU | B | 171 | −0.514 | −77.821 | 9.605 | 1.00 | 50.00 | C |
| ATOM | 645 | CD2 | LEU | B | 171 | 0.044 | −75.461 | 8.934 | 1.00 | 50.00 | C |
| ATOM | 646 | N | LEU | B | 172 | 4.125 | −76.175 | 10.843 | 1.00 | 50.00 | N |
| ATOM | 647 | CA | LEU | B | 172 | 5.022 | −75.398 | 11.692 | 1.00 | 50.00 | C |
| ATOM | 648 | C | LEU | B | 172 | 5.838 | −74.275 | 11.061 | 1.00 | 50.00 | C |
| ATOM | 649 | O | LEU | B | 172 | 5.643 | −73.145 | 11.504 | 1.00 | 50.00 | O |
| ATOM | 650 | CB | LEU | B | 172 | 5.839 | −76.452 | 12.439 | 1.00 | 50.00 | C |
| ATOM | 651 | CG | LEU | B | 172 | 6.364 | −76.011 | 13.803 | 1.00 | 50.00 | C |
| ATOM | 652 | CD1 | LEU | B | 172 | 7.632 | −75.177 | 13.648 | 1.00 | 50.00 | C |
| ATOM | 653 | CD2 | LEU | B | 172 | 5.359 | −75.229 | 14.649 | 1.00 | 50.00 | C |
| ATOM | 654 | N | SER | B | 173 | 6.683 | −74.503 | 10.066 | 1.00 | 50.00 | N |
| ATOM | 655 | CA | SER | B | 173 | 7.358 | −73.434 | 9.324 | 1.00 | 50.00 | C |
| ATOM | 656 | C | SER | B | 173 | 6.510 | −72.280 | 8.801 | 1.00 | 50.00 | C |
| ATOM | 657 | O | SER | B | 173 | 6.934 | −71.126 | 8.794 | 1.00 | 50.00 | O |
| ATOM | 658 | CB | SER | B | 173 | 8.194 | −74.001 | 8.176 | 1.00 | 50.00 | C |
| ATOM | 659 | OG | SER | B | 173 | 9.356 | −74.637 | 8.687 | 1.00 | 50.00 | O |
| ATOM | 660 | N | THR | B | 174 | 5.294 | −72.610 | 8.405 | 1.00 | 50.00 | N |
| ATOM | 661 | CA | THR | B | 174 | 4.260 | −71.677 | 7.965 | 1.00 | 50.00 | C |
| ATOM | 662 | C | THR | B | 174 | 3.466 | −70.953 | 9.044 | 1.00 | 50.00 | C |
| ATOM | 663 | O | THR | B | 174 | 3.201 | −69.762 | 8.889 | 1.00 | 50.00 | O |
| ATOM | 664 | CB | THR | B | 174 | 3.291 | −72.527 | 7.147 | 1.00 | 50.00 | C |
| ATOM | 665 | OG1 | THR | B | 174 | 3.974 | −73.364 | 6.229 | 1.00 | 50.00 | O |
| ATOM | 666 | CG2 | THR | B | 174 | 2.469 | −71.561 | 6.305 | 1.00 | 50.00 | C |
| ATOM | 667 | N | ASN | B | 175 | 3.010 | −71.638 | 10.079 | 1.00 | 50.00 | N |
| ATOM | 668 | CA | ASN | B | 175 | 2.543 | −71.037 | 11.329 | 1.00 | 50.00 | C |
| ATOM | 669 | C | ASN | B | 175 | 3.486 | −69.973 | 11.881 | 1.00 | 50.00 | C |
| ATOM | 670 | O | ASN | B | 175 | 3.035 | −68.844 | 12.069 | 1.00 | 50.00 | O |
| ATOM | 671 | CB | ASN | B | 175 | 2.260 | −72.179 | 12.307 | 1.00 | 50.00 | C |
| ATOM | 672 | CG | ASN | B | 175 | 0.953 | −72.935 | 12.113 | 1.00 | 50.00 | C |
| ATOM | 673 | OD1 | ASN | B | 175 | −0.111 | −72.366 | 11.873 | 1.00 | 50.00 | O |
| ATOM | 674 | ND2 | ASN | B | 175 | 0.990 | −74.249 | 12.240 | 1.00 | 50.00 | N |
| ATOM | 675 | N | LYS | B | 176 | 4.774 | −70.258 | 11.990 | 1.00 | 50.00 | N |
| ATOM | 676 | CA | LYS | B | 176 | 5.841 | −69.267 | 12.147 | 1.00 | 50.00 | C |
| ATOM | 677 | C | LYS | B | 176 | 5.933 | −68.141 | 11.126 | 1.00 | 50.00 | C |
| ATOM | 678 | O | LYS | B | 176 | 6.159 | −66.998 | 11.521 | 1.00 | 50.00 | O |
| ATOM | 679 | CB | LYS | B | 176 | 7.212 | −69.941 | 12.144 | 1.00 | 50.00 | C |
| ATOM | 680 | CG | LYS | B | 176 | 7.479 | −70.864 | 13.331 | 1.00 | 50.00 | C |
| ATOM | 681 | CD | LYS | B | 176 | 8.930 | −71.338 | 13.317 | 1.00 | 50.00 | C |
| ATOM | 682 | CE | LYS | B | 176 | 9.216 | −72.261 | 14.499 | 1.00 | 50.00 | C |
| ATOM | 683 | NZ | LYS | B | 176 | 10.403 | −73.090 | 14.244 | 1.00 | 50.00 | N |
| ATOM | 684 | N | ALA | B | 177 | 5.772 | −68.428 | 9.844 | 1.00 | 50.00 | N |
| ATOM | 685 | CA | ALA | B | 177 | 5.553 | −67.418 | 8.811 | 1.00 | 50.00 | C |
| ATOM | 686 | C | ALA | B | 177 | 4.360 | −66.508 | 9.101 | 1.00 | 50.00 | C |
| ATOM | 687 | O | ALA | B | 177 | 4.579 | −65.298 | 9.083 | 1.00 | 50.00 | O |
| ATOM | 688 | CB | ALA | B | 177 | 5.636 | −68.167 | 7.480 | 1.00 | 50.00 | C |
| ATOM | 689 | N | VAL | B | 178 | 3.160 | −66.986 | 9.399 | 1.00 | 50.00 | N |
| ATOM | 690 | CA | VAL | B | 178 | 2.018 | −66.185 | 9.853 | 1.00 | 50.00 | C |
| ATOM | 691 | C | VAL | B | 178 | 2.330 | −65.324 | 11.075 | 1.00 | 50.00 | C |
| ATOM | 692 | O | VAL | B | 178 | 1.980 | −64.145 | 11.036 | 1.00 | 50.00 | O |
| ATOM | 693 | CB | VAL | B | 178 | 0.691 | −66.926 | 10.052 | 1.00 | 50.00 | C |
| ATOM | 694 | CG1 | VAL | B | 178 | −0.488 | −65.987 | 10.312 | 1.00 | 50.00 | C |
| ATOM | 695 | CG2 | VAL | B | 178 | 0.164 | −67.867 | 8.968 | 1.00 | 50.00 | C |
| ATOM | 696 | N | VAL | B | 179 | 2.957 | −65.833 | 12.124 | 1.00 | 50.00 | N |
| ATOM | 697 | CA | VAL | B | 179 | 3.440 | −65.073 | 13.280 | 1.00 | 50.00 | C |
| ATOM | 698 | C | VAL | B | 179 | 4.375 | −63.923 | 12.926 | 1.00 | 50.00 | C |
| ATOM | 699 | O | VAL | B | 179 | 4.065 | −62.790 | 13.292 | 1.00 | 50.00 | O |
| ATOM | 700 | CB | VAL | B | 179 | 4.150 | −65.956 | 14.310 | 1.00 | 50.00 | C |
| ATOM | 701 | CG1 | VAL | B | 179 | 4.710 | −65.260 | 15.550 | 1.00 | 50.00 | C |
| ATOM | 702 | CG2 | VAL | B | 179 | 3.296 | −67.091 | 14.865 | 1.00 | 50.00 | C |
| ATOM | 703 | N | SER | B | 180 | 5.501 | −64.207 | 12.291 | 1.00 | 50.00 | N |
| ATOM | 704 | CA | SER | B | 180 | 6.459 | −63.216 | 11.796 | 1.00 | 50.00 | C |
| ATOM | 705 | C | SER | B | 180 | 5.841 | −62.032 | 11.066 | 1.00 | 50.00 | C |
| ATOM | 706 | O | SER | B | 180 | 6.120 | −60.876 | 11.381 | 1.00 | 50.00 | O |
| ATOM | 707 | CB | SER | B | 180 | 7.476 | −63.899 | 10.882 | 1.00 | 50.00 | C |
| ATOM | 708 | OG | SER | B | 180 | 8.320 | −64.764 | 11.629 | 1.00 | 50.00 | O |
| ATOM | 709 | N | LEU | B | 181 | 4.928 | −62.351 | 10.165 | 1.00 | 50.00 | N |
| ATOM | 710 | CA | LEU | B | 181 | 4.040 | −61.369 | 9.554 | 1.00 | 50.00 | C |
| ATOM | 711 | C | LEU | B | 181 | 3.024 | −60.642 | 10.425 | 1.00 | 50.00 | C |
| ATOM | 712 | O | LEU | B | 181 | 2.992 | −59.417 | 10.331 | 1.00 | 50.00 | O |
| ATOM | 713 | CB | LEU | B | 181 | 3.376 | −62.108 | 8.395 | 1.00 | 50.00 | C |
| ATOM | 714 | CG | LEU | B | 181 | 2.688 | −61.102 | 7.478 | 1.00 | 50.00 | C |
| ATOM | 715 | CD1 | LEU | B | 181 | 3.636 | −60.010 | 6.988 | 1.00 | 50.00 | C |
| ATOM | 716 | CD2 | LEU | B | 181 | 1.934 | −61.957 | 6.467 | 1.00 | 50.00 | C |
| ATOM | 717 | N | SER | B | 182 | 2.198 | −61.319 | 11.207 | 1.00 | 50.00 | N |
| ATOM | 718 | CA | SER | B | 182 | 1.350 | −60.704 | 12.230 | 1.00 | 50.00 | C |
| ATOM | 719 | C | SER | B | 182 | 2.032 | −59.674 | 13.125 | 1.00 | 50.00 | C |
| ATOM | 720 | O | SER | B | 182 | 1.475 | −58.598 | 13.332 | 1.00 | 50.00 | O |
| ATOM | 721 | CB | SER | B | 182 | 0.663 | −61.757 | 13.101 | 1.00 | 50.00 | C |
| ATOM | 722 | OG | SER | B | 182 | −0.178 | −62.655 | 12.392 | 1.00 | 50.00 | O |
| ATOM | 723 | N | ASN | B | 183 | 3.242 | −59.967 | 13.574 | 1.00 | 50.00 | N |
| ATOM | 724 | CA | ASN | B | 183 | 4.174 | −59.027 | 14.199 | 1.00 | 50.00 | C |
| ATOM | 725 | C | ASN | B | 183 | 4.640 | −57.864 | 13.329 | 1.00 | 50.00 | C |
| ATOM | 726 | O | ASN | B | 183 | 4.623 | −56.735 | 13.818 | 1.00 | 50.00 | O |
| ATOM | 727 | CB | ASN | B | 183 | 5.371 | −59.796 | 14.759 | 1.00 | 50.00 | C |
| ATOM | 728 | CG | ASN | B | 183 | 5.111 | −60.882 | 15.796 | 1.00 | 50.00 | C |
| ATOM | 729 | OD1 | ASN | B | 183 | 4.149 | −60.846 | 16.563 | 1.00 | 50.00 | O |
| ATOM | 730 | ND2 | ASN | B | 183 | 5.963 | −61.889 | 15.845 | 1.00 | 50.00 | N |
| ATOM | 731 | N | GLY | B | 184 | 5.019 | −58.065 | 12.076 | 1.00 | 50.00 | N |
| ATOM | 732 | CA | GLY | B | 184 | 5.175 | −56.996 | 11.089 | 1.00 | 50.00 | C |
| ATOM | 733 | C | GLY | B | 184 | 4.003 | −56.049 | 10.852 | 1.00 | 50.00 | C |
| ATOM | 734 | O | GLY | B | 184 | 4.227 | −54.839 | 10.826 | 1.00 | 50.00 | O |
| ATOM | 735 | N | VAL | B | 185 | 2.787 | −56.558 | 10.733 | 1.00 | 50.00 | N |
| ATOM | 736 | CA | VAL | B | 185 | 1.537 | −55.794 | 10.779 | 1.00 | 50.00 | C |
| ATOM | 737 | C | VAL | B | 185 | 1.312 | −55.045 | 12.087 | 1.00 | 50.00 | C |
| ATOM | 738 | O | VAL | B | 185 | 0.875 | −53.898 | 12.028 | 1.00 | 50.00 | O |
| ATOM | 739 | CB | VAL | B | 185 | 0.260 | −56.587 | 10.481 | 1.00 | 50.00 | C |
| ATOM | 740 | CG1 | VAL | B | 185 | −0.998 | −55.753 | 10.234 | 1.00 | 50.00 | C |
| ATOM | 741 | CG2 | VAL | B | 185 | 0.396 | −57.399 | 9.198 | 1.00 | 50.00 | C |
| ATOM | 742 | N | SER | B | 186 | 1.552 | −55.659 | 13.234 | 1.00 | 50.00 | N |
| ATOM | 743 | CA | SER | B | 186 | 1.497 | −55.019 | 14.548 | 1.00 | 50.00 | C |
| ATOM | 744 | C | SER | B | 186 | 2.304 | −53.736 | 14.711 | 1.00 | 50.00 | C |
| ATOM | 745 | O | SER | B | 186 | 1.742 | −52.718 | 15.112 | 1.00 | 50.00 | O |
| ATOM | 746 | CB | SER | B | 186 | 1.940 | −56.031 | 15.605 | 1.00 | 50.00 | C |
| ATOM | 747 | OG | SER | B | 186 | 1.404 | −55.662 | 16.867 | 1.00 | 50.00 | O |
| ATOM | 748 | N | VAL | B | 187 | 3.582 | −53.791 | 14.374 | 1.00 | 50.00 | N |
| ATOM | 749 | CA | VAL | B | 187 | 4.480 | −52.638 | 14.299 | 1.00 | 50.00 | C |
| ATOM | 750 | C | VAL | B | 187 | 4.040 | −51.622 | 13.250 | 1.00 | 50.00 | C |
| ATOM | 751 | O | VAL | B | 187 | 4.055 | −50.424 | 13.523 | 1.00 | 50.00 | O |
| ATOM | 752 | CB | VAL | B | 187 | 5.936 | −53.074 | 14.119 | 1.00 | 50.00 | C |
| ATOM | 753 | CG1 | VAL | B | 187 | 6.919 | −51.905 | 14.049 | 1.00 | 50.00 | C |
| ATOM | 754 | CG2 | VAL | B | 187 | 6.426 | −54.029 | 15.208 | 1.00 | 50.00 | C |
| ATOM | 755 | N | LEU | B | 188 | 3.649 | −52.055 | 12.064 | 1.00 | 50.00 | N |
| ATOM | 756 | CA | LEU | B | 188 | 3.015 | −51.192 | 11.070 | 1.00 | 50.00 | C |
| ATOM | 757 | C | LEU | B | 188 | 1.733 | −50.460 | 11.450 | 1.00 | 50.00 | C |
| ATOM | 758 | O | LEU | B | 188 | 1.610 | −49.271 | 11.161 | 1.00 | 50.00 | O |
| ATOM | 759 | CB | LEU | B | 188 | 2.811 | −52.095 | 9.854 | 1.00 | 50.00 | C |
| ATOM | 760 | CG | LEU | B | 188 | 2.318 | −51.367 | 8.607 | 1.00 | 50.00 | C |
| ATOM | 761 | CD1 | LEU | B | 188 | 3.250 | −50.209 | 8.265 | 1.00 | 50.00 | C |
| ATOM | 762 | CD2 | LEU | B | 188 | 2.186 | −52.424 | 7.516 | 1.00 | 50.00 | C |
| ATOM | 763 | N | THR | B | 189 | 0.801 | −51.140 | 12.097 | 1.00 | 50.00 | N |
| ATOM | 764 | CA | THR | B | 189 | −0.293 | −50.536 | 12.857 | 1.00 | 50.00 | C |
| ATOM | 765 | C | THR | B | 189 | 0.234 | −49.542 | 13.884 | 1.00 | 50.00 | C |
| ATOM | 766 | O | THR | B | 189 | −0.140 | −48.381 | 13.727 | 1.00 | 50.00 | O |
| ATOM | 767 | CB | THR | B | 189 | −1.254 | −51.563 | 13.460 | 1.00 | 50.00 | C |
| ATOM | 768 | OG1 | THR | B | 189 | −1.612 | −52.577 | 12.533 | 1.00 | 50.00 | O |
| ATOM | 769 | CG2 | THR | B | 189 | −2.561 | −50.950 | 13.958 | 1.00 | 50.00 | C |
| ATOM | 770 | N | SER | B | 190 | 1.107 | −49.896 | 14.816 | 1.00 | 50.00 | N |
| ATOM | 771 | CA | SER | B | 190 | 1.863 | −48.974 | 15.667 | 1.00 | 50.00 | C |
| ATOM | 772 | C | SER | B | 190 | 2.415 | −47.690 | 15.058 | 1.00 | 50.00 | C |
| ATOM | 773 | O | SER | B | 190 | 2.386 | −46.630 | 15.683 | 1.00 | 50.00 | O |
| ATOM | 774 | CB | SER | B | 190 | 2.811 | −49.736 | 16.593 | 1.00 | 50.00 | C |
| ATOM | 775 | OG | SER | B | 190 | 4.135 | −49.827 | 16.087 | 1.00 | 50.00 | O |
| ATOM | 776 | N | LYS | B | 191 | 2.864 | −47.770 | 13.817 | 1.00 | 50.00 | N |
| ATOM | 777 | CA | LYS | B | 191 | 3.247 | −46.631 | 12.985 | 1.00 | 50.00 | C |
| ATOM | 778 | C | LYS | B | 191 | 2.083 | −45.808 | 12.443 | 1.00 | 50.00 | C |
| ATOM | 779 | O | LYS | B | 191 | 2.115 | −44.593 | 12.634 | 1.00 | 50.00 | O |
| ATOM | 780 | CB | LYS | B | 191 | 4.271 | −47.172 | 11.987 | 1.00 | 50.00 | C |
| ATOM | 781 | CG | LYS | B | 191 | 5.645 | −47.600 | 12.507 | 1.00 | 50.00 | C |
| ATOM | 782 | CD | LYS | B | 191 | 6.338 | −46.715 | 13.544 | 1.00 | 50.00 | C |
| ATOM | 783 | CE | LYS | B | 191 | 6.066 | −47.055 | 15.008 | 1.00 | 50.00 | C |
| ATOM | 784 | NZ | LYS | B | 191 | 6.691 | −46.091 | 15.924 | 1.00 | 50.00 | N |
| ATOM | 785 | N | VAL | B | 192 | 1.019 | −46.380 | 11.900 | 1.00 | 50.00 | N |
| ATOM | 786 | CA | VAL | B | 192 | −0.326 | −45.798 | 11.866 | 1.00 | 50.00 | C |
| ATOM | 787 | C | VAL | B | 192 | −0.819 | −45.134 | 13.151 | 1.00 | 50.00 | C |
| ATOM | 788 | O | VAL | B | 192 | −1.300 | −44.009 | 13.025 | 1.00 | 50.00 | O |
| ATOM | 789 | CB | VAL | B | 192 | −1.298 | −46.748 | 11.155 | 1.00 | 50.00 | C |
| ATOM | 790 | CG1 | VAL | B | 192 | −2.728 | −46.859 | 11.686 | 1.00 | 50.00 | C |
| ATOM | 791 | CG2 | VAL | B | 192 | −1.392 | −46.516 | 9.646 | 1.00 | 50.00 | C |
| ATOM | 792 | N | LEU | B | 193 | −0.713 | −45.684 | 14.352 | 1.00 | 50.00 | N |
| ATOM | 793 | CA | LEU | B | 193 | −1.010 | −45.014 | 15.621 | 1.00 | 50.00 | C |
| ATOM | 794 | C | LEU | B | 193 | −0.290 | −43.681 | 15.810 | 1.00 | 50.00 | C |
| ATOM | 795 | O | LEU | B | 193 | −0.946 | −42.662 | 16.024 | 1.00 | 50.00 | O |
| ATOM | 796 | CB | LEU | B | 193 | −0.714 | −45.837 | 16.880 | 1.00 | 50.00 | C |
| ATOM | 797 | CG | LEU | B | 193 | −1.533 | −47.047 | 17.325 | 1.00 | 50.00 | C |
| ATOM | 798 | CD1 | LEU | B | 193 | −1.577 | −48.182 | 16.305 | 1.00 | 50.00 | C |
| ATOM | 799 | CD2 | LEU | B | 193 | −1.024 | −47.664 | 18.626 | 1.00 | 50.00 | C |
| ATOM | 800 | N | ASP | B | 194 | 1.033 | −43.701 | 15.768 | 1.00 | 50.00 | N |
| ATOM | 801 | CA | ASP | B | 194 | 1.899 | −42.562 | 16.075 | 1.00 | 50.00 | C |
| ATOM | 802 | C | ASP | B | 194 | 1.877 | −41.473 | 15.014 | 1.00 | 50.00 | C |
| ATOM | 803 | O | ASP | B | 194 | 1.640 | −40.327 | 15.392 | 1.00 | 50.00 | O |
| ATOM | 804 | CB | ASP | B | 194 | 3.361 | −42.964 | 16.264 | 1.00 | 50.00 | C |
| ATOM | 805 | CG | ASP | B | 194 | 3.670 | −43.685 | 17.566 | 1.00 | 50.00 | C |
| ATOM | 806 | OD1 | ASP | B | 194 | 3.412 | −43.115 | 18.648 | 1.00 | 50.00 | O |
| ATOM | 807 | OD2 | ASP | B | 194 | 4.184 | −44.824 | 17.514 | 1.00 | 50.00 | O |
| ATOM | 808 | N | LEU | B | 195 | 2.067 | −41.806 | 13.747 | 1.00 | 50.00 | N |
| ATOM | 809 | CA | LEU | B | 195 | 1.757 | −40.919 | 12.627 | 1.00 | 50.00 | C |
| ATOM | 810 | C | LEU | B | 195 | 0.433 | −40.193 | 12.821 | 1.00 | 50.00 | C |
| ATOM | 811 | O | LEU | B | 195 | 0.456 | −38.974 | 12.984 | 1.00 | 50.00 | O |
| ATOM | 812 | CB | LEU | B | 195 | 1.848 | −41.654 | 11.288 | 1.00 | 50.00 | C |
| ATOM | 813 | CG | LEU | B | 195 | 1.715 | −40.785 | 10.037 | 1.00 | 50.00 | C |
| ATOM | 814 | CD1 | LEU | B | 195 | 0.306 | −40.295 | 9.709 | 1.00 | 50.00 | C |
| ATOM | 815 | CD2 | LEU | B | 195 | 2.745 | −39.662 | 9.973 | 1.00 | 50.00 | C |
| ATOM | 816 | N | LYS | B | 196 | −0.676 | −40.912 | 12.862 | 1.00 | 50.00 | N |
| ATOM | 817 | CA | LYS | B | 196 | −2.014 | −40.336 | 12.984 | 1.00 | 50.00 | C |
| ATOM | 818 | C | LYS | B | 196 | −2.193 | −39.229 | 14.016 | 1.00 | 50.00 | C |
| ATOM | 819 | O | LYS | B | 196 | −2.460 | −38.082 | 13.658 | 1.00 | 50.00 | O |
| ATOM | 820 | CB | LYS | B | 196 | −2.973 | −41.508 | 13.201 | 1.00 | 50.00 | C |
| ATOM | 821 | CG | LYS | B | 196 | −4.438 | −41.160 | 12.956 | 1.00 | 50.00 | C |
| ATOM | 822 | CD | LYS | B | 196 | −4.696 | −41.226 | 11.453 | 1.00 | 50.00 | C |
| ATOM | 823 | CE | LYS | B | 196 | −6.068 | −40.645 | 11.129 | 1.00 | 50.00 | C |
| ATOM | 824 | NZ | LYS | B | 196 | −6.608 | −40.373 | 9.788 | 1.00 | 50.00 | N |
| ATOM | 825 | N | ASN | B | 197 | −1.942 | −39.584 | 15.265 | 1.00 | 50.00 | N |
| ATOM | 826 | CA | ASN | B | 197 | −1.875 | −38.663 | 16.399 | 1.00 | 50.00 | C |
| ATOM | 827 | C | ASN | B | 197 | −0.860 | −37.532 | 16.285 | 1.00 | 50.00 | C |
| ATOM | 828 | O | ASN | B | 197 | −1.224 | −36.410 | 16.632 | 1.00 | 50.00 | O |
| ATOM | 829 | CB | ASN | B | 197 | −1.671 | −39.491 | 17.669 | 1.00 | 50.00 | C |
| ATOM | 830 | CG | ASN | B | 197 | −2.769 | −40.458 | 18.093 | 1.00 | 50.00 | C |
| ATOM | 831 | OD1 | ASN | B | 197 | −2.498 | −41.607 | 18.440 | 1.00 | 50.00 | O |
| ATOM | 832 | ND2 | ASN | B | 197 | −4.024 | −40.048 | 18.094 | 1.00 | 50.00 | N |
| ATOM | 833 | N | TYR | B | 198 | 0.336 | −37.753 | 15.761 | 1.00 | 50.00 | N |
| ATOM | 834 | CA | TYR | B | 198 | 1.268 | −36.696 | 15.367 | 1.00 | 50.00 | C |
| ATOM | 835 | C | TYR | B | 198 | 0.678 | −35.601 | 14.487 | 1.00 | 50.00 | C |
| ATOM | 836 | O | TYR | B | 198 | 0.804 | −34.440 | 14.870 | 1.00 | 50.00 | O |
| ATOM | 837 | CB | TYR | B | 198 | 2.542 | −37.259 | 14.734 | 1.00 | 50.00 | C |
| ATOM | 838 | CG | TYR | B | 198 | 3.606 | −36.211 | 14.495 | 1.00 | 50.00 | C |
| ATOM | 839 | CD1 | TYR | B | 198 | 4.474 | −35.892 | 15.477 | 1.00 | 50.00 | C |
| ATOM | 840 | CD2 | TYR | B | 198 | 3.649 | −35.539 | 13.325 | 1.00 | 50.00 | C |
| ATOM | 841 | CE1 | TYR | B | 198 | 5.442 | −34.980 | 15.258 | 1.00 | 50.00 | C |
| ATOM | 842 | CE2 | TYR | B | 198 | 4.610 | −34.620 | 13.105 | 1.00 | 50.00 | C |
| ATOM | 843 | CZ | TYR | B | 198 | 5.548 | −34.397 | 14.047 | 1.00 | 50.00 | C |
| ATOM | 844 | OH | TYR | B | 198 | 6.696 | −33.726 | 13.718 | 1.00 | 50.00 | O |
| ATOM | 845 | N | ILE | B | 199 | 0.110 | −35.920 | 13.336 | 1.00 | 50.00 | N |
| ATOM | 846 | CA | ILE | B | 199 | −0.529 | −34.969 | 12.424 | 1.00 | 50.00 | C |
| ATOM | 847 | C | ILE | B | 199 | −1.586 | −34.102 | 13.094 | 1.00 | 50.00 | C |
| ATOM | 848 | O | ILE | B | 199 | −1.648 | −32.902 | 12.838 | 1.00 | 50.00 | O |
| ATOM | 849 | CB | ILE | B | 199 | −1.127 | −35.707 | 11.224 | 1.00 | 50.00 | C |
| ATOM | 850 | CG1 | ILE | B | 199 | −0.062 | −36.065 | 10.193 | 1.00 | 50.00 | C |
| ATOM | 851 | CG2 | ILE | B | 199 | −2.163 | −34.961 | 10.387 | 1.00 | 50.00 | C |
| ATOM | 852 | CD1 | ILE | B | 199 | 0.841 | −37.204 | 10.650 | 1.00 | 50.00 | C |
| ATOM | 853 | N | ASP | B | 200 | −2.436 | −34.705 | 13.904 | 1.00 | 50.00 | N |
| ATOM | 854 | CA | ASP | B | 200 | −3.438 | −34.030 | 14.727 | 1.00 | 50.00 | C |
| ATOM | 855 | C | ASP | B | 200 | −2.770 | −33.009 | 15.640 | 1.00 | 50.00 | C |
| ATOM | 856 | O | ASP | B | 200 | −2.885 | −31.815 | 15.371 | 1.00 | 50.00 | O |
| ATOM | 857 | CB | ASP | B | 200 | −4.267 | −35.124 | 15.402 | 1.00 | 50.00 | C |
| ATOM | 858 | CG | ASP | B | 200 | −4.830 | −36.202 | 14.485 | 1.00 | 50.00 | C |
| ATOM | 859 | OD1 | ASP | B | 200 | −4.966 | −35.944 | 13.269 | 1.00 | 50.00 | O |
| ATOM | 860 | OD2 | ASP | B | 200 | −5.063 | −37.339 | 14.947 | 1.00 | 50.00 | O |
| ATOM | 861 | N | LYS | B | 201 | −1.903 | −33.435 | 16.543 | 1.00 | 50.00 | N |
| ATOM | 862 | CA | LYS | B | 201 | −0.946 | −32.574 | 17.240 | 1.00 | 50.00 | C |
| ATOM | 863 | C | LYS | B | 201 | −0.030 | −31.628 | 16.473 | 1.00 | 50.00 | C |
| ATOM | 864 | O | LYS | B | 201 | 0.580 | −30.793 | 17.140 | 1.00 | 50.00 | O |
| ATOM | 865 | CB | LYS | B | 201 | 0.056 | −33.420 | 18.025 | 1.00 | 50.00 | C |
| ATOM | 866 | CG | LYS | B | 201 | −0.551 | −34.345 | 19.075 | 1.00 | 50.00 | C |
| ATOM | 867 | CD | LYS | B | 201 | 0.592 | −35.067 | 19.783 | 1.00 | 50.00 | C |
| ATOM | 868 | CE | LYS | B | 201 | 0.015 | −36.169 | 20.667 | 1.00 | 50.00 | C |
| ATOM | 869 | NZ | LYS | B | 201 | 1.103 | −36.733 | 21.477 | 1.00 | 50.00 | N |
| ATOM | 870 | N | GLN | B | 202 | 0.138 | −31.770 | 15.169 | 1.00 | 50.00 | N |
| ATOM | 871 | CA | GLN | B | 202 | 0.887 | −30.829 | 14.339 | 1.00 | 50.00 | C |
| ATOM | 872 | C | GLN | B | 202 | 0.129 | −29.879 | 13.424 | 1.00 | 50.00 | C |
| ATOM | 873 | O | GLN | B | 202 | 0.605 | −28.775 | 13.166 | 1.00 | 50.00 | O |
| ATOM | 874 | CB | GLN | B | 202 | 1.874 | −31.524 | 13.406 | 1.00 | 50.00 | C |
| ATOM | 875 | CG | GLN | B | 202 | 3.116 | −30.634 | 13.386 | 1.00 | 50.00 | C |
| ATOM | 876 | CD | GLN | B | 202 | 4.205 | −31.287 | 14.218 | 1.00 | 50.00 | C |
| ATOM | 877 | OE1 | GLN | B | 202 | 4.009 | −31.766 | 15.334 | 1.00 | 50.00 | O |
| ATOM | 878 | NE2 | GLN | B | 202 | 5.383 | −31.290 | 13.626 | 1.00 | 50.00 | N |
| ATOM | 879 | N | LEU | B | 203 | −1.040 | −30.280 | 12.965 | 1.00 | 50.00 | N |
| ATOM | 880 | CA | LEU | B | 203 | −2.029 | −29.398 | 12.353 | 1.00 | 50.00 | C |
| ATOM | 881 | C | LEU | B | 203 | −2.737 | −28.543 | 13.396 | 1.00 | 50.00 | C |
| ATOM | 882 | O | LEU | B | 203 | −2.837 | −27.360 | 13.085 | 1.00 | 50.00 | O |
| ATOM | 883 | CB | LEU | B | 203 | −2.937 | −30.306 | 11.529 | 1.00 | 50.00 | C |
| ATOM | 884 | CG | LEU | B | 203 | −2.380 | −30.798 | 10.191 | 1.00 | 50.00 | C |
| ATOM | 885 | CD1 | LEU | B | 203 | −0.955 | −31.334 | 10.072 | 1.00 | 50.00 | C |
| ATOM | 886 | CD2 | LEU | B | 203 | −3.316 | −31.934 | 9.795 | 1.00 | 50.00 | C |
| ATOM | 887 | N | LEU | B | 204 | −3.101 | −28.997 | 14.585 | 1.00 | 50.00 | N |
| ATOM | 888 | CA | LEU | B | 204 | −3.522 | −28.191 | 15.733 | 1.00 | 50.00 | C |
| ATOM | 889 | C | LEU | B | 204 | −2.642 | −27.076 | 16.302 | 1.00 | 50.00 | C |
| ATOM | 890 | O | LEU | B | 204 | −3.223 | −26.042 | 16.628 | 1.00 | 50.00 | O |
| ATOM | 891 | CB | LEU | B | 204 | −3.911 | −29.183 | 16.831 | 1.00 | 50.00 | C |
| ATOM | 892 | CG | LEU | B | 204 | −5.261 | −29.901 | 16.768 | 1.00 | 50.00 | C |
| ATOM | 893 | CD1 | LEU | B | 204 | −5.704 | −30.587 | 15.476 | 1.00 | 50.00 | C |
| ATOM | 894 | CD2 | LEU | B | 204 | −5.324 | −30.902 | 17.921 | 1.00 | 50.00 | C |
| ATOM | 895 | N | PRO | B | 205 | −1.332 | −27.129 | 16.500 | 1.00 | 50.00 | N |
| ATOM | 896 | CA | PRO | B | 205 | −0.435 | −25.977 | 16.599 | 1.00 | 50.00 | C |
| ATOM | 897 | C | PRO | B | 205 | −0.531 | −25.086 | 15.367 | 1.00 | 50.00 | C |
| ATOM | 898 | O | PRO | B | 205 | −1.018 | −23.979 | 15.584 | 1.00 | 50.00 | O |
| ATOM | 899 | CB | PRO | B | 205 | 0.944 | −26.564 | 16.894 | 1.00 | 50.00 | C |
| ATOM | 900 | CG | PRO | B | 205 | 0.976 | −27.919 | 16.198 | 1.00 | 50.00 | C |
| ATOM | 901 | CD | PRO | B | 205 | −0.508 | −28.249 | 16.063 | 1.00 | 50.00 | C |
| ATOM | 902 | N | ILE | B | 206 | −0.293 | −25.521 | 14.139 | 1.00 | 50.00 | N |
| ATOM | 903 | CA | ILE | B | 206 | −0.679 | −24.823 | 12.910 | 1.00 | 50.00 | C |
| ATOM | 904 | C | ILE | B | 206 | −2.046 | −24.141 | 12.927 | 1.00 | 50.00 | C |
| ATOM | 905 | O | ILE | B | 206 | −2.103 | −22.986 | 12.507 | 1.00 | 50.00 | O |
| ATOM | 906 | CB | ILE | B | 206 | −0.440 | −25.648 | 11.641 | 1.00 | 50.00 | C |
| ATOM | 907 | CG1 | ILE | B | 206 | 0.959 | −26.253 | 11.496 | 1.00 | 50.00 | C |
| ATOM | 908 | CG2 | ILE | B | 206 | −0.818 | −24.832 | 10.403 | 1.00 | 50.00 | C |
| ATOM | 909 | CD1 | ILE | B | 206 | 1.131 | −27.452 | 10.563 | 1.00 | 50.00 | C |
| ATOM | 910 | N | VAL | B | 207 | −3.121 | −24.773 | 13.369 | 1.00 | 50.00 | N |
| ATOM | 911 | CA | VAL | B | 207 | −4.469 | −24.222 | 13.527 | 1.00 | 50.00 | C |
| ATOM | 912 | C | VAL | B | 207 | −4.431 | −22.893 | 14.277 | 1.00 | 50.00 | C |
| ATOM | 913 | O | VAL | B | 207 | −4.766 | −21.865 | 13.690 | 1.00 | 50.00 | O |
| ATOM | 914 | CB | VAL | B | 207 | −5.416 | −25.273 | 14.119 | 1.00 | 50.00 | C |
| ATOM | 915 | CG1 | VAL | B | 207 | −6.742 | −24.735 | 14.653 | 1.00 | 50.00 | C |
| ATOM | 916 | CG2 | VAL | B | 207 | −5.841 | −26.436 | 13.224 | 1.00 | 50.00 | C |
| ATOM | 917 | N | ASN | B | 208 | −4.000 | −22.909 | 15.528 | 1.00 | 50.00 | N |
| ATOM | 918 | CA | ASN | B | 208 | −3.856 | −21.718 | 16.366 | 1.00 | 50.00 | C |
| ATOM | 919 | C | ASN | B | 208 | −2.625 | −20.841 | 16.166 | 1.00 | 50.00 | C |
| ATOM | 920 | O | ASN | B | 208 | −2.692 | −19.654 | 16.482 | 1.00 | 50.00 | O |
| ATOM | 921 | CB | ASN | B | 208 | −3.983 | −22.116 | 17.837 | 1.00 | 50.00 | C |
| ATOM | 922 | CG | ASN | B | 208 | −5.341 | −22.593 | 18.334 | 1.00 | 50.00 | C |
| ATOM | 923 | OD1 | ASN | B | 208 | −5.448 | −23.619 | 19.004 | 1.00 | 50.00 | O |
| ATOM | 924 | ND2 | ASN | B | 208 | −6.407 | −21.866 | 18.052 | 1.00 | 50.00 | N |
| ATOM | 925 | N | LYS | B | 209 | −1.525 | −21.338 | 15.625 | 1.00 | 50.00 | N |
| ATOM | 926 | CA | LYS | B | 209 | −0.465 | −20.539 | 15.009 | 1.00 | 50.00 | C |
| ATOM | 927 | C | LYS | B | 209 | −0.980 | −19.631 | 13.896 | 1.00 | 50.00 | C |
| ATOM | 928 | O | LYS | B | 209 | −0.756 | −18.424 | 13.982 | 1.00 | 50.00 | O |
| ATOM | 929 | CB | LYS | B | 209 | 0.693 | −21.423 | 14.546 | 1.00 | 50.00 | C |
| ATOM | 930 | CG | LYS | B | 209 | 1.520 | −22.154 | 15.602 | 1.00 | 50.00 | C |
| ATOM | 931 | CD | LYS | B | 209 | 2.557 | −23.118 | 15.028 | 1.00 | 50.00 | C |
| ATOM | 932 | CE | LYS | B | 209 | 3.339 | −23.814 | 16.140 | 1.00 | 50.00 | C |
| ATOM | 933 | NZ | LYS | B | 209 | 4.148 | −24.934 | 15.636 | 1.00 | 50.00 | N |
| ATOM | 934 | N | GLN | B | 210 | −1.754 | −20.139 | 12.949 | 1.00 | 50.00 | N |
| ATOM | 935 | CA | GLN | B | 210 | −2.599 | −19.346 | 12.056 | 1.00 | 50.00 | C |
| ATOM | 936 | C | GLN | B | 210 | −3.521 | −18.321 | 12.710 | 1.00 | 50.00 | C |
| ATOM | 937 | O | GLN | B | 210 | −3.602 | −17.215 | 12.178 | 1.00 | 50.00 | O |
| ATOM | 938 | CB | GLN | B | 210 | −3.349 | −20.266 | 11.089 | 1.00 | 50.00 | C |
| ATOM | 939 | CG | GLN | B | 210 | −3.281 | −19.892 | 9.610 | 1.00 | 50.00 | C |
| ATOM | 940 | CD | GLN | B | 210 | −3.997 | −18.596 | 9.262 | 1.00 | 50.00 | C |
| ATOM | 941 | OE1 | GLN | B | 210 | −5.126 | −18.338 | 9.675 | 1.00 | 50.00 | O |
| ATOM | 942 | NE2 | GLN | B | 210 | −3.335 | −17.744 | 8.503 | 1.00 | 50.00 | N |
| ATOM | 943 | N | SER | B | 211 | −4.163 | −18.611 | 13.832 | 1.00 | 50.00 | N |
| ATOM | 944 | CA | SER | B | 211 | −4.929 | −17.645 | 14.623 | 1.00 | 50.00 | C |
| ATOM | 945 | C | SER | B | 211 | −4.138 | −16.485 | 15.216 | 1.00 | 50.00 | C |
| ATOM | 946 | O | SER | B | 211 | −4.529 | −15.338 | 15.004 | 1.00 | 50.00 | O |
| ATOM | 947 | CB | SER | B | 211 | −5.718 | −18.326 | 15.741 | 1.00 | 50.00 | C |
| ATOM | 948 | OG | SER | B | 211 | −6.657 | −19.268 | 15.242 | 1.00 | 50.00 | O |
| ATOM | 949 | N | CYS | B | 212 | −3.044 | −16.722 | 15.922 | 1.00 | 50.00 | N |
| ATOM | 950 | CA | CYS | B | 212 | −2.107 | −15.694 | 16.381 | 1.00 | 50.00 | C |
| ATOM | 951 | C | CYS | B | 212 | −1.661 | −14.677 | 15.337 | 1.00 | 50.00 | C |
| ATOM | 952 | O | CYS | B | 212 | −1.788 | −13.477 | 15.574 | 1.00 | 50.00 | O |
| ATOM | 953 | CB | CYS | B | 212 | −0.923 | −16.407 | 17.035 | 1.00 | 50.00 | C |
| ATOM | 954 | SG | CYS | B | 212 | −0.227 | −15.461 | 18.398 | 1.00 | 50.00 | S |
| ATOM | 955 | N | SER | B | 213 | −1.266 | −15.145 | 14.163 | 1.00 | 50.00 | N |
| ATOM | 956 | CA | SER | B | 213 | −1.135 | −14.337 | 12.952 | 1.00 | 50.00 | C |
| ATOM | 957 | C | SER | B | 213 | −2.386 | −13.605 | 12.480 | 1.00 | 50.00 | C |
| ATOM | 958 | O | SER | B | 213 | −2.338 | −12.377 | 12.523 | 1.00 | 50.00 | O |
| ATOM | 959 | CB | SER | B | 213 | −0.464 | −15.172 | 11.862 | 1.00 | 50.00 | C |
| ATOM | 960 | OG | SER | B | 213 | 0.920 | −15.294 | 12.154 | 1.00 | 50.00 | O |
| ATOM | 961 | N | ILE | B | 214 | −3.481 | −14.255 | 12.114 | 1.00 | 50.00 | N |
| ATOM | 962 | CA | ILE | B | 214 | −4.786 | −13.646 | 11.837 | 1.00 | 50.00 | C |
| ATOM | 963 | C | ILE | B | 214 | −5.207 | −12.453 | 12.691 | 1.00 | 50.00 | C |
| ATOM | 964 | O | ILE | B | 214 | −5.633 | −11.415 | 12.184 | 1.00 | 50.00 | O |
| ATOM | 965 | CB | ILE | B | 214 | −5.885 | −14.700 | 11.649 | 1.00 | 50.00 | C |
| ATOM | 966 | CG1 | ILE | B | 214 | −6.588 | −14.580 | 10.295 | 1.00 | 50.00 | C |
| ATOM | 967 | CG2 | ILE | B | 214 | −6.939 | −14.825 | 12.752 | 1.00 | 50.00 | C |
| ATOM | 968 | CD1 | ILE | B | 214 | −7.476 | −15.750 | 9.878 | 1.00 | 50.00 | C |
| ATOM | 969 | N | SER | B | 215 | −5.085 | −12.637 | 13.995 | 1.00 | 50.00 | N |
| ATOM | 970 | CA | SER | B | 215 | −5.400 | −11.658 | 15.034 | 1.00 | 50.00 | C |
| ATOM | 971 | C | SER | B | 215 | −4.374 | −10.538 | 15.141 | 1.00 | 50.00 | C |
| ATOM | 972 | O | SER | B | 215 | −4.789 | −9.382 | 15.204 | 1.00 | 50.00 | O |
| ATOM | 973 | CB | SER | B | 215 | −5.521 | −12.402 | 16.363 | 1.00 | 50.00 | C |
| ATOM | 974 | OG | SER | B | 215 | −6.549 | −13.378 | 16.287 | 1.00 | 50.00 | O |
| ATOM | 975 | N | ASN | B | 216 | −3.081 | −10.816 | 15.102 | 1.00 | 50.00 | N |
| ATOM | 976 | CA | ASN | B | 216 | −2.048 | −9.794 | 14.939 | 1.00 | 50.00 | C |
| ATOM | 977 | C | ASN | B | 216 | −2.246 | −8.904 | 13.721 | 1.00 | 50.00 | C |
| ATOM | 978 | O | ASN | B | 216 | −2.595 | −7.752 | 13.973 | 1.00 | 50.00 | O |
| ATOM | 979 | CB | ASN | B | 216 | −0.632 | −10.353 | 15.078 | 1.00 | 50.00 | C |
| ATOM | 980 | CG | ASN | B | 216 | −0.216 | −10.943 | 16.416 | 1.00 | 50.00 | C |
| ATOM | 981 | OD1 | ASN | B | 216 | 0.542 | −11.903 | 16.544 | 1.00 | 50.00 | O |
| ATOM | 982 | ND2 | ASN | B | 216 | −0.751 | −10.324 | 17.449 | 1.00 | 50.00 | N |
| ATOM | 983 | N | ILE | B | 217 | −2.210 | −9.413 | 12.500 | 1.00 | 50.00 | N |
| ATOM | 984 | CA | ILE | B | 217 | −2.766 | −8.790 | 11.295 | 1.00 | 50.00 | C |
| ATOM | 985 | C | ILE | B | 217 | −3.954 | −7.844 | 11.454 | 1.00 | 50.00 | C |
| ATOM | 986 | O | ILE | B | 217 | −4.077 | −6.910 | 10.663 | 1.00 | 50.00 | O |
| ATOM | 987 | CB | ILE | B | 217 | −3.054 | −9.816 | 10.194 | 1.00 | 50.00 | C |
| ATOM | 988 | CG1 | ILE | B | 217 | −1.898 | −10.761 | 9.861 | 1.00 | 50.00 | C |
| ATOM | 989 | CG2 | ILE | B | 217 | −3.481 | −9.186 | 8.867 | 1.00 | 50.00 | C |
| ATOM | 990 | CD1 | ILE | B | 217 | −2.256 | −12.045 | 9.118 | 1.00 | 50.00 | C |
| ATOM | 991 | N | GLU | B | 218 | −4.813 | −8.035 | 12.441 | 1.00 | 50.00 | N |
| ATOM | 992 | CA | GLU | B | 218 | −5.817 | −7.064 | 12.874 | 1.00 | 50.00 | C |
| ATOM | 993 | C | GLU | B | 218 | −5.548 | −5.908 | 13.818 | 1.00 | 50.00 | C |
| ATOM | 994 | O | GLU | B | 218 | −5.779 | −4.736 | 13.521 | 1.00 | 50.00 | O |
| ATOM | 995 | CB | GLU | B | 218 | −7.042 | −7.837 | 13.371 | 1.00 | 50.00 | C |
| ATOM | 996 | CG | GLU | B | 218 | −7.833 | −8.549 | 12.278 | 1.00 | 50.00 | C |
| ATOM | 997 | CD | GLU | B | 218 | −8.051 | −7.437 | 11.269 | 1.00 | 50.00 | C |
| ATOM | 998 | OE1 | GLU | B | 218 | −7.183 | −7.477 | 10.371 | 1.00 | 50.00 | O |
| ATOM | 999 | OE2 | GLU | B | 218 | −8.861 | −6.496 | 11.412 | 1.00 | 50.00 | O |
| ATOM | 1000 | N | THR | B | 219 | −5.148 | −6.337 | 14.999 | 1.00 | 50.00 | N |
| ATOM | 1001 | CA | THR | B | 219 | −4.764 | −5.484 | 16.120 | 1.00 | 50.00 | C |
| ATOM | 1002 | C | THR | B | 219 | −3.773 | −4.421 | 15.664 | 1.00 | 50.00 | C |
| ATOM | 1003 | O | THR | B | 219 | −4.094 | −3.234 | 15.696 | 1.00 | 50.00 | O |
| ATOM | 1004 | CB | THR | B | 219 | −4.304 | −6.465 | 17.198 | 1.00 | 50.00 | C |
| ATOM | 1005 | OG1 | THR | B | 219 | −3.099 | −7.143 | 16.868 | 1.00 | 50.00 | O |
| ATOM | 1006 | CG2 | THR | B | 219 | −5.350 | −7.497 | 17.620 | 1.00 | 50.00 | C |
| ATOM | 1007 | N | VAL | B | 220 | −2.690 | −4.888 | 15.068 | 1.00 | 50.00 | N |
| ATOM | 1008 | CA | VAL | B | 220 | −1.809 | −4.156 | 14.164 | 1.00 | 50.00 | C |
| ATOM | 1009 | C | VAL | B | 220 | −2.280 | −3.097 | 13.176 | 1.00 | 50.00 | C |
| ATOM | 1010 | O | VAL | B | 220 | −1.483 | −2.230 | 12.825 | 1.00 | 50.00 | O |
| ATOM | 1011 | CB | VAL | B | 220 | −0.963 | −5.170 | 13.396 | 1.00 | 50.00 | C |
| ATOM | 1012 | CG1 | VAL | B | 220 | −0.148 | −6.202 | 14.165 | 1.00 | 50.00 | C |
| ATOM | 1013 | CG2 | VAL | B | 220 | −1.311 | −5.756 | 12.032 | 1.00 | 50.00 | C |
| ATOM | 1014 | N | ILE | B | 221 | −3.443 | −3.189 | 12.558 | 1.00 | 50.00 | N |
| ATOM | 1015 | CA | ILE | B | 221 | −4.003 | −2.093 | 11.765 | 1.00 | 50.00 | C |
| ATOM | 1016 | C | ILE | B | 221 | −4.759 | −1.126 | 12.667 | 1.00 | 50.00 | C |
| ATOM | 1017 | O | ILE | B | 221 | −4.620 | 0.092 | 12.575 | 1.00 | 50.00 | O |
| ATOM | 1018 | CB | ILE | B | 221 | −4.799 | −2.652 | 10.583 | 1.00 | 50.00 | C |
| ATOM | 1019 | CG1 | ILE | B | 221 | −3.915 | −3.142 | 9.436 | 1.00 | 50.00 | C |
| ATOM | 1020 | CG2 | ILE | B | 221 | −5.783 | −1.720 | 9.880 | 1.00 | 50.00 | C |
| ATOM | 1021 | CD1 | ILE | B | 221 | −2.818 | −4.123 | 9.833 | 1.00 | 50.00 | C |
| ATOM | 1022 | N | GLU | B | 222 | −5.580 | −1.716 | 13.518 | 1.00 | 50.00 | N |
| ATOM | 1023 | CA | GLU | B | 222 | −6.428 | −1.088 | 14.529 | 1.00 | 50.00 | C |
| ATOM | 1024 | C | GLU | B | 222 | −5.814 | −0.306 | 15.687 | 1.00 | 50.00 | C |
| ATOM | 1025 | O | GLU | B | 222 | −6.444 | 0.427 | 16.447 | 1.00 | 50.00 | O |
| ATOM | 1026 | CB | GLU | B | 222 | −7.310 | −2.265 | 14.950 | 1.00 | 50.00 | C |
| ATOM | 1027 | CG | GLU | B | 222 | −8.781 | −2.020 | 15.272 | 1.00 | 50.00 | C |
| ATOM | 1028 | CD | GLU | B | 222 | −9.015 | −0.729 | 16.030 | 1.00 | 50.00 | C |
| ATOM | 1029 | OE1 | GLU | B | 222 | −9.129 | 0.235 | 15.245 | 1.00 | 50.00 | O |
| ATOM | 1030 | OE2 | GLU | B | 222 | −8.919 | −0.629 | 17.272 | 1.00 | 50.00 | O |
| ATOM | 1031 | N | PHE | B | 223 | −4.496 | −0.304 | 15.708 | 1.00 | 50.00 | N |
| ATOM | 1032 | CA | PHE | B | 223 | −3.602 | 0.482 | 16.554 | 1.00 | 50.00 | C |
| ATOM | 1033 | C | PHE | B | 223 | −2.798 | 1.447 | 15.674 | 1.00 | 50.00 | C |
| ATOM | 1034 | O | PHE | B | 223 | −2.457 | 2.523 | 16.165 | 1.00 | 50.00 | O |
| ATOM | 1035 | CB | PHE | B | 223 | −2.858 | −0.680 | 17.221 | 1.00 | 50.00 | C |
| ATOM | 1036 | CG | PHE | B | 223 | −2.810 | −1.578 | 18.441 | 1.00 | 50.00 | C |
| ATOM | 1037 | CD1 | PHE | B | 223 | −2.740 | −1.107 | 19.703 | 1.00 | 50.00 | C |
| ATOM | 1038 | CD2 | PHE | B | 223 | −2.500 | −2.879 | 18.246 | 1.00 | 50.00 | C |
| ATOM | 1039 | CE1 | PHE | B | 223 | −2.585 | −1.949 | 20.746 | 1.00 | 50.00 | C |
| ATOM | 1040 | CE2 | PHE | B | 223 | −2.421 | −3.751 | 19.270 | 1.00 | 50.00 | C |
| ATOM | 1041 | CZ | PHE | B | 223 | −2.513 | −3.278 | 20.527 | 1.00 | 50.00 | C |
| ATOM | 1042 | N | GLN | B | 224 | −2.454 | 1.179 | 14.421 | 1.00 | 50.00 | N |
| ATOM | 1043 | CA | GLN | B | 224 | −1.772 | 2.030 | 13.442 | 1.00 | 50.00 | C |
| ATOM | 1044 | C | GLN | B | 224 | −2.486 | 3.241 | 12.867 | 1.00 | 50.00 | C |
| ATOM | 1045 | O | GLN | B | 224 | −1.988 | 4.366 | 12.920 | 1.00 | 50.00 | O |
| ATOM | 1046 | CB | GLN | B | 224 | −1.400 | 1.227 | 12.195 | 1.00 | 50.00 | C |
| ATOM | 1047 | CG | GLN | B | 224 | −0.191 | 0.305 | 12.164 | 1.00 | 50.00 | C |
| ATOM | 1048 | CD | GLN | B | 224 | 1.060 | 1.120 | 12.420 | 1.00 | 50.00 | C |
| ATOM | 1049 | OE1 | GLN | B | 224 | 1.166 | 2.343 | 12.337 | 1.00 | 50.00 | O |
| ATOM | 1050 | NE2 | GLN | B | 224 | 2.011 | 0.291 | 12.794 | 1.00 | 50.00 | N |
| ATOM | 1051 | N | GLN | B | 225 | −3.639 | 2.967 | 12.281 | 1.00 | 50.00 | N |
| ATOM | 1052 | CA | GLN | B | 225 | −4.607 | 4.012 | 11.972 | 1.00 | 50.00 | C |
| ATOM | 1053 | C | GLN | B | 225 | −5.034 | 4.843 | 13.181 | 1.00 | 50.00 | C |
| ATOM | 1054 | O | GLN | B | 225 | −5.613 | 5.910 | 12.993 | 1.00 | 50.00 | O |
| ATOM | 1055 | CB | GLN | B | 225 | −5.781 | 3.428 | 11.192 | 1.00 | 50.00 | C |
| ATOM | 1056 | CG | GLN | B | 225 | −5.926 | 2.564 | 9.946 | 1.00 | 50.00 | C |
| ATOM | 1057 | CD | GLN | B | 225 | −5.595 | 3.312 | 8.668 | 1.00 | 50.00 | C |
| ATOM | 1058 | OE1 | GLN | B | 225 | −5.848 | 4.501 | 8.482 | 1.00 | 50.00 | O |
| ATOM | 1059 | NE2 | GLN | B | 225 | −5.135 | 2.522 | 7.721 | 1.00 | 50.00 | N |
| ATOM | 1060 | N | LYS | B | 226 | −4.836 | 4.443 | 14.428 | 1.00 | 50.00 | N |
| ATOM | 1061 | CA | LYS | B | 226 | −5.122 | 5.234 | 15.623 | 1.00 | 50.00 | C |
| ATOM | 1062 | C | LYS | B | 226 | −4.074 | 6.331 | 15.720 | 1.00 | 50.00 | C |
| ATOM | 1063 | O | LYS | B | 226 | −4.489 | 7.486 | 15.787 | 1.00 | 50.00 | O |
| ATOM | 1064 | CB | LYS | B | 226 | −5.223 | 4.430 | 16.921 | 1.00 | 50.00 | C |
| ATOM | 1065 | CG | LYS | B | 226 | −5.576 | 5.245 | 18.167 | 1.00 | 50.00 | C |
| ATOM | 1066 | CD | LYS | B | 226 | −7.048 | 5.647 | 18.173 | 1.00 | 50.00 | C |
| ATOM | 1067 | CE | LYS | B | 226 | −7.540 | 6.707 | 19.154 | 1.00 | 50.00 | C |
| ATOM | 1068 | NZ | LYS | B | 226 | −9.004 | 6.819 | 19.119 | 1.00 | 50.00 | N |
| ATOM | 1069 | N | ASN | B | 227 | −2.792 | 6.009 | 15.673 | 1.00 | 50.00 | N |
| ATOM | 1070 | CA | ASN | B | 227 | −1.717 | 6.998 | 15.646 | 1.00 | 50.00 | C |
| ATOM | 1071 | C | ASN | B | 227 | −1.842 | 8.047 | 14.547 | 1.00 | 50.00 | C |
| ATOM | 1072 | O | ASN | B | 227 | −2.080 | 9.192 | 14.927 | 1.00 | 50.00 | O |
| ATOM | 1073 | CB | ASN | B | 227 | −0.397 | 6.232 | 15.722 | 1.00 | 50.00 | C |
| ATOM | 1074 | CG | ASN | B | 227 | 0.668 | 7.028 | 16.460 | 1.00 | 50.00 | C |
| ATOM | 1075 | OD1 | ASN | B | 227 | 0.443 | 7.744 | 17.435 | 1.00 | 50.00 | O |
| ATOM | 1076 | ND2 | ASN | B | 227 | 1.899 | 6.946 | 15.998 | 1.00 | 50.00 | N |
| ATOM | 1077 | N | ASN | B | 228 | −1.878 | 7.747 | 13.258 | 1.00 | 50.00 | N |
| ATOM | 1078 | CA | ASN | B | 228 | −2.326 | 8.701 | 12.241 | 1.00 | 50.00 | C |
| ATOM | 1079 | C | ASN | B | 228 | −3.594 | 9.509 | 12.506 | 1.00 | 50.00 | C |
| ATOM | 1080 | O | ASN | B | 228 | −3.597 | 10.700 | 12.199 | 1.00 | 50.00 | O |
| ATOM | 1081 | CB | ASN | B | 228 | −2.331 | 8.083 | 10.842 | 1.00 | 50.00 | C |
| ATOM | 1082 | CG | ASN | B | 228 | −3.241 | 6.909 | 10.514 | 1.00 | 50.00 | C |
| ATOM | 1083 | OD1 | ASN | B | 228 | −2.816 | 5.939 | 9.888 | 1.00 | 50.00 | O |
| ATOM | 1084 | ND2 | ASN | B | 228 | −4.519 | 7.017 | 10.824 | 1.00 | 50.00 | N |
| ATOM | 1085 | N | ARG | B | 229 | −4.627 | 8.928 | 13.097 | 1.00 | 50.00 | N |
| ATOM | 1086 | CA | ARG | B | 229 | −5.879 | 9.587 | 13.468 | 1.00 | 50.00 | C |
| ATOM | 1087 | C | ARG | B | 229 | −5.646 | 10.618 | 14.566 | 1.00 | 50.00 | C |
| ATOM | 1088 | O | ARG | B | 229 | −5.713 | 11.813 | 14.285 | 1.00 | 50.00 | O |
| ATOM | 1089 | CB | ARG | B | 229 | −6.923 | 8.539 | 13.856 | 1.00 | 50.00 | C |
| ATOM | 1090 | CG | ARG | B | 229 | −8.391 | 8.962 | 13.870 | 1.00 | 50.00 | C |
| ATOM | 1091 | CD | ARG | B | 229 | −9.389 | 8.202 | 14.739 | 1.00 | 50.00 | C |
| ATOM | 1092 | NE | ARG | B | 229 | −9.149 | 6.763 | 14.660 | 1.00 | 50.00 | N |
| ATOM | 1093 | CZ | ARG | B | 229 | −9.417 | 5.919 | 15.639 | 1.00 | 50.00 | C |
| ATOM | 1094 | NH1 | ARG | B | 229 | −10.314 | 6.097 | 16.588 | 1.00 | 50.00 | N |
| ATOM | 1095 | NH2 | ARG | B | 229 | −8.738 | 4.791 | 15.667 | 1.00 | 50.00 | N |
| ATOM | 1096 | N | LEU | B | 230 | −5.248 | 10.182 | 15.749 | 1.00 | 50.00 | N |
| ATOM | 1097 | CA | LEU | B | 230 | −4.625 | 10.978 | 16.805 | 1.00 | 50.00 | C |
| ATOM | 1098 | C | LEU | B | 230 | −3.721 | 12.146 | 16.432 | 1.00 | 50.00 | C |
| ATOM | 1099 | O | LEU | B | 230 | −3.915 | 13.263 | 16.907 | 1.00 | 50.00 | O |
| ATOM | 1100 | CB | LEU | B | 230 | −3.826 | 9.986 | 17.651 | 1.00 | 50.00 | C |
| ATOM | 1101 | CG | LEU | B | 230 | −4.002 | 10.121 | 19.160 | 1.00 | 50.00 | C |
| ATOM | 1102 | CD1 | LEU | B | 230 | −5.401 | 9.675 | 19.578 | 1.00 | 50.00 | C |
| ATOM | 1103 | CD2 | LEU | B | 230 | −2.965 | 9.183 | 19.772 | 1.00 | 50.00 | C |
| ATOM | 1104 | N | LEU | B | 231 | −2.756 | 11.871 | 15.570 | 1.00 | 50.00 | N |
| ATOM | 1105 | CA | LEU | B | 231 | −1.834 | 12.856 | 15.012 | 1.00 | 50.00 | C |
| ATOM | 1106 | C | LEU | B | 231 | −2.590 | 13.835 | 14.125 | 1.00 | 50.00 | C |
| ATOM | 1107 | O | LEU | B | 231 | −2.636 | 14.981 | 14.568 | 1.00 | 50.00 | O |
| ATOM | 1108 | CB | LEU | B | 231 | −0.674 | 12.115 | 14.350 | 1.00 | 50.00 | C |
| ATOM | 1109 | CG | LEU | B | 231 | 0.378 | 11.534 | 15.295 | 1.00 | 50.00 | C |
| ATOM | 1110 | CD1 | LEU | B | 231 | 0.069 | 10.617 | 16.476 | 1.00 | 50.00 | C |
| ATOM | 1111 | CD2 | LEU | B | 231 | 1.410 | 10.814 | 14.431 | 1.00 | 50.00 | C |
| ATOM | 1112 | N | GLU | B | 232 | −3.294 | 13.462 | 13.067 | 1.00 | 50.00 | N |
| ATOM | 1113 | CA | GLU | B | 232 | −4.275 | 14.307 | 12.382 | 1.00 | 50.00 | C |
| ATOM | 1114 | C | GLU | B | 232 | −5.114 | 15.259 | 13.224 | 1.00 | 50.00 | C |
| ATOM | 1115 | O | GLU | B | 232 | −5.109 | 16.469 | 13.002 | 1.00 | 50.00 | O |
| ATOM | 1116 | CB | GLU | B | 232 | −5.291 | 13.483 | 11.588 | 1.00 | 50.00 | C |
| ATOM | 1117 | CG | GLU | B | 232 | −5.898 | 14.162 | 10.362 | 1.00 | 50.00 | C |
| ATOM | 1118 | CD | GLU | B | 232 | −7.247 | 14.848 | 10.516 | 1.00 | 50.00 | C |
| ATOM | 1119 | OE1 | GLU | B | 232 | −7.540 | 15.473 | 11.558 | 1.00 | 50.00 | O |
| ATOM | 1120 | OE2 | GLU | B | 232 | −8.062 | 14.724 | 9.577 | 1.00 | 50.00 | O |
| ATOM | 1121 | N | ILE | B | 233 | −5.828 | 14.676 | 14.172 | 1.00 | 50.00 | N |
| ATOM | 1122 | CA | ILE | B | 233 | −6.760 | 15.335 | 15.083 | 1.00 | 50.00 | C |
| ATOM | 1123 | C | ILE | B | 233 | −5.947 | 16.371 | 15.849 | 1.00 | 50.00 | C |
| ATOM | 1124 | O | ILE | B | 233 | −5.971 | 17.564 | 15.551 | 1.00 | 50.00 | O |
| ATOM | 1125 | CB | ILE | B | 233 | −7.464 | 14.254 | 15.911 | 1.00 | 50.00 | C |
| ATOM | 1126 | CG1 | ILE | B | 233 | −8.329 | 13.307 | 15.077 | 1.00 | 50.00 | C |
| ATOM | 1127 | CG2 | ILE | B | 233 | −8.286 | 14.930 | 17.006 | 1.00 | 50.00 | C |
| ATOM | 1128 | CD1 | ILE | B | 233 | −9.168 | 12.271 | 15.822 | 1.00 | 50.00 | C |
| ATOM | 1129 | N | THR | B | 234 | −5.154 | 15.853 | 16.768 | 1.00 | 50.00 | N |
| ATOM | 1130 | CA | THR | B | 234 | −4.463 | 16.662 | 17.765 | 1.00 | 50.00 | C |
| ATOM | 1131 | C | THR | B | 234 | −3.004 | 16.876 | 17.367 | 1.00 | 50.00 | C |
| ATOM | 1132 | O | THR | B | 234 | −2.088 | 16.730 | 18.174 | 1.00 | 50.00 | O |
| ATOM | 1133 | CB | THR | B | 234 | −4.889 | 15.919 | 19.032 | 1.00 | 50.00 | C |
| ATOM | 1134 | OG1 | THR | B | 234 | −6.284 | 15.903 | 19.295 | 1.00 | 50.00 | O |
| ATOM | 1135 | CG2 | THR | B | 234 | −4.420 | 16.726 | 20.232 | 1.00 | 50.00 | C |
| ATOM | 1136 | N | ARG | B | 235 | −2.755 | 17.315 | 16.143 | 1.00 | 50.00 | N |
| ATOM | 1137 | CA | ARG | B | 235 | −1.438 | 17.677 | 15.616 | 1.00 | 50.00 | C |
| ATOM | 1138 | C | ARG | B | 235 | −0.821 | 18.898 | 16.293 | 1.00 | 50.00 | C |
| ATOM | 1139 | O | ARG | B | 235 | 0.307 | 18.742 | 16.758 | 1.00 | 50.00 | O |
| ATOM | 1140 | CB | ARG | B | 235 | −1.424 | 17.651 | 14.086 | 1.00 | 50.00 | C |
| ATOM | 1141 | CG | ARG | B | 235 | −2.490 | 18.472 | 13.364 | 1.00 | 50.00 | C |
| ATOM | 1142 | CD | ARG | B | 235 | −2.427 | 18.402 | 11.839 | 1.00 | 50.00 | C |
| ATOM | 1143 | NE | ARG | B | 235 | −3.212 | 19.494 | 11.264 | 1.00 | 50.00 | N |
| ATOM | 1144 | CZ | ARG | B | 235 | −2.726 | 20.424 | 10.461 | 1.00 | 50.00 | C |
| ATOM | 1145 | NH1 | ARG | B | 235 | −1.668 | 20.225 | 9.704 | 1.00 | 50.00 | N |
| ATOM | 1146 | NH2 | ARG | B | 235 | −3.238 | 21.638 | 10.420 | 1.00 | 50.00 | N |
| ATOM | 1147 | N | GLU | B | 236 | −1.500 | 20.025 | 16.455 | 1.00 | 50.00 | N |
| ATOM | 1148 | CA | GLU | B | 236 | −1.159 | 21.162 | 17.319 | 1.00 | 50.00 | C |
| ATOM | 1149 | C | GLU | B | 236 | −0.413 | 20.884 | 18.616 | 1.00 | 50.00 | C |
| ATOM | 1150 | O | GLU | B | 236 | 0.760 | 21.222 | 18.772 | 1.00 | 50.00 | O |
| ATOM | 1151 | CB | GLU | B | 236 | −2.243 | 22.157 | 17.754 | 1.00 | 50.00 | C |
| ATOM | 1152 | CG | GLU | B | 236 | −3.603 | 21.702 | 18.279 | 1.00 | 50.00 | C |
| ATOM | 1153 | CD | GLU | B | 236 | −4.259 | 20.960 | 17.133 | 1.00 | 50.00 | C |
| ATOM | 1154 | OE1 | GLU | B | 236 | −4.335 | 19.713 | 17.151 | 1.00 | 50.00 | O |
| ATOM | 1155 | OE2 | GLU | B | 236 | −4.608 | 21.643 | 16.148 | 1.00 | 50.00 | O |
| ATOM | 1156 | N | PHE | B | 237 | −1.147 | 20.312 | 19.556 | 1.00 | 50.00 | N |
| ATOM | 1157 | CA | PHE | B | 237 | −0.689 | 19.983 | 20.904 | 1.00 | 50.00 | C |
| ATOM | 1158 | C | PHE | B | 237 | 0.572 | 19.123 | 20.899 | 1.00 | 50.00 | C |
| ATOM | 1159 | O | PHE | B | 237 | 1.473 | 19.297 | 21.719 | 1.00 | 50.00 | O |
| ATOM | 1160 | CB | PHE | B | 237 | −1.936 | 19.473 | 21.634 | 1.00 | 50.00 | C |
| ATOM | 1161 | CG | PHE | B | 237 | −2.077 | 18.039 | 22.101 | 1.00 | 50.00 | C |
| ATOM | 1162 | CD1 | PHE | B | 237 | −1.478 | 17.003 | 21.479 | 1.00 | 50.00 | C |
| ATOM | 1163 | CD2 | PHE | B | 237 | −2.989 | 17.737 | 23.045 | 1.00 | 50.00 | C |
| ATOM | 1164 | CE1 | PHE | B | 237 | −1.655 | 15.718 | 21.833 | 1.00 | 50.00 | C |
| ATOM | 1165 | CE2 | PHE | B | 237 | −3.302 | 16.456 | 23.327 | 1.00 | 50.00 | C |
| ATOM | 1166 | CZ | PHE | B | 237 | −2.636 | 15.455 | 22.716 | 1.00 | 50.00 | C |
| ATOM | 1167 | N | SER | B | 238 | 0.589 | 18.191 | 19.959 | 1.00 | 50.00 | N |
| ATOM | 1168 | CA | SER | B | 238 | 1.523 | 17.075 | 19.829 | 1.00 | 50.00 | C |
| ATOM | 1169 | C | SER | B | 238 | 2.841 | 17.517 | 19.211 | 1.00 | 50.00 | C |
| ATOM | 1170 | O | SER | B | 238 | 3.790 | 16.734 | 19.233 | 1.00 | 50.00 | O |
| ATOM | 1171 | CB | SER | B | 238 | 0.912 | 15.940 | 19.004 | 1.00 | 50.00 | C |
| ATOM | 1172 | OG | SER | B | 238 | 0.929 | 16.157 | 17.600 | 1.00 | 50.00 | O |
| ATOM | 1173 | N | VAL | B | 239 | 2.854 | 18.704 | 18.627 | 1.00 | 50.00 | N |
| ATOM | 1174 | CA | VAL | B | 239 | 4.013 | 19.357 | 18.033 | 1.00 | 50.00 | C |
| ATOM | 1175 | C | VAL | B | 239 | 4.555 | 20.531 | 18.846 | 1.00 | 50.00 | C |
| ATOM | 1176 | O | VAL | B | 239 | 4.027 | 20.879 | 19.902 | 1.00 | 50.00 | O |
| ATOM | 1177 | CB | VAL | B | 239 | 3.719 | 19.597 | 16.554 | 1.00 | 50.00 | C |
| ATOM | 1178 | CG1 | VAL | B | 239 | 3.438 | 18.381 | 15.675 | 1.00 | 50.00 | C |
| ATOM | 1179 | CG2 | VAL | B | 239 | 2.778 | 20.786 | 16.381 | 1.00 | 50.00 | C |
| ATOM | 1180 | N | ASN | B | 240 | 5.535 | 21.229 | 18.294 | 1.00 | 50.00 | N |
| ATOM | 1181 | CA | ASN | B | 240 | 5.924 | 22.575 | 18.719 | 1.00 | 50.00 | C |
| ATOM | 1182 | C | ASN | B | 240 | 4.867 | 23.639 | 18.432 | 1.00 | 50.00 | C |
| ATOM | 1183 | O | ASN | B | 240 | 4.666 | 24.483 | 19.304 | 1.00 | 50.00 | O |
| ATOM | 1184 | CB | ASN | B | 240 | 7.280 | 23.045 | 18.188 | 1.00 | 50.00 | C |
| ATOM | 1185 | CG | ASN | B | 240 | 8.633 | 22.426 | 18.506 | 1.00 | 50.00 | C |
| ATOM | 1186 | OD1 | ASN | B | 240 | 9.498 | 22.330 | 17.636 | 1.00 | 50.00 | O |
| ATOM | 1187 | ND2 | ASN | B | 240 | 8.888 | 22.016 | 19.734 | 1.00 | 50.00 | N |
| ATOM | 1188 | N | ALA | B | 241 | 4.205 | 23.663 | 17.285 | 1.00 | 50.00 | N |
| ATOM | 1189 | CA | ALA | B | 241 | 3.222 | 24.684 | 16.917 | 1.00 | 50.00 | C |
| ATOM | 1190 | C | ALA | B | 241 | 1.780 | 24.347 | 17.270 | 1.00 | 50.00 | C |
| ATOM | 1191 | O | ALA | B | 241 | 1.318 | 23.225 | 17.070 | 1.00 | 50.00 | O |
| ATOM | 1192 | CB | ALA | B | 241 | 3.168 | 24.878 | 15.402 | 1.00 | 50.00 | C |
| ATOM | 1193 | N | GLY | B | 242 | 1.003 | 25.384 | 17.532 | 1.00 | 50.00 | N |
| ATOM | 1194 | CA | GLY | B | 242 | −0.430 | 25.359 | 17.245 | 1.00 | 50.00 | C |
| ATOM | 1195 | C | GLY | B | 242 | −0.856 | 25.274 | 15.783 | 1.00 | 50.00 | C |
| ATOM | 1196 | O | GLY | B | 242 | −0.106 | 25.436 | 14.821 | 1.00 | 50.00 | O |
| ATOM | 1197 | N | VAL | B | 243 | −2.128 | 24.930 | 15.689 | 1.00 | 50.00 | N |
| ATOM | 1198 | CA | VAL | B | 243 | −2.830 | 24.264 | 14.590 | 1.00 | 50.00 | C |
| ATOM | 1199 | C | VAL | B | 243 | −4.285 | 24.282 | 15.050 | 1.00 | 50.00 | C |
| ATOM | 1200 | O | VAL | B | 243 | −4.653 | 24.620 | 16.175 | 1.00 | 50.00 | O |
| ATOM | 1201 | CB | VAL | B | 243 | −2.388 | 22.820 | 14.334 | 1.00 | 50.00 | C |
| ATOM | 1202 | CG1 | VAL | B | 243 | −3.178 | 21.897 | 13.410 | 1.00 | 50.00 | C |
| ATOM | 1203 | CG2 | VAL | B | 243 | −0.922 | 22.643 | 13.964 | 1.00 | 50.00 | C |
| ATOM | 1204 | N | THR | B | 244 | −5.178 | 23.800 | 14.201 | 1.00 | 50.00 | N |
| ATOM | 1205 | CA | THR | B | 244 | −6.548 | 23.451 | 14.577 | 1.00 | 50.00 | C |
| ATOM | 1206 | C | THR | B | 244 | −6.919 | 22.054 | 14.065 | 1.00 | 50.00 | C |
| ATOM | 1207 | O | THR | B | 244 | −6.142 | 21.456 | 13.322 | 1.00 | 50.00 | O |
| ATOM | 1208 | CB | THR | B | 244 | −7.324 | 24.771 | 14.483 | 1.00 | 50.00 | C |
| ATOM | 1209 | OG1 | THR | B | 244 | −6.573 | 25.978 | 14.463 | 1.00 | 50.00 | O |
| ATOM | 1210 | CG2 | THR | B | 244 | −8.261 | 25.034 | 15.659 | 1.00 | 50.00 | C |
| ATOM | 1211 | N | THR | B | 245 | −7.966 | 21.340 | 14.460 | 1.00 | 50.00 | N |
| ATOM | 1212 | CA | THR | B | 245 | −7.883 | 20.031 | 15.119 | 1.00 | 50.00 | C |
| ATOM | 1213 | C | THR | B | 245 | −8.995 | 18.990 | 15.209 | 1.00 | 50.00 | C |
| ATOM | 1214 | O | THR | B | 245 | −8.784 | 17.971 | 15.865 | 1.00 | 50.00 | O |
| ATOM | 1215 | CB | THR | B | 245 | −7.364 | 20.260 | 16.539 | 1.00 | 50.00 | C |
| ATOM | 1216 | OG1 | THR | B | 245 | −7.163 | 19.146 | 17.397 | 1.00 | 50.00 | O |
| ATOM | 1217 | CG2 | THR | B | 245 | −7.823 | 21.442 | 17.391 | 1.00 | 50.00 | C |
| ATOM | 1218 | N | PRO | B | 246 | −9.905 | 18.748 | 14.283 | 1.00 | 50.00 | N |
| ATOM | 1219 | CA | PRO | B | 246 | −11.254 | 19.310 | 14.260 | 1.00 | 50.00 | C |
| ATOM | 1220 | C | PRO | B | 246 | −12.429 | 18.523 | 14.821 | 1.00 | 50.00 | C |
| ATOM | 1221 | O | PRO | B | 246 | −13.474 | 19.109 | 15.098 | 1.00 | 50.00 | O |
| ATOM | 1222 | CB | PRO | B | 246 | −11.553 | 19.431 | 12.766 | 1.00 | 50.00 | C |
| ATOM | 1223 | CG | PRO | B | 246 | −10.751 | 18.266 | 12.194 | 1.00 | 50.00 | C |
| ATOM | 1224 | CD | PRO | B | 246 | −9.462 | 18.200 | 13.008 | 1.00 | 50.00 | C |
| ATOM | 1225 | N | VAL | B | 247 | −12.416 | 17.204 | 14.760 | 1.00 | 50.00 | N |
| ATOM | 1226 | CA | VAL | B | 247 | −11.585 | 16.317 | 15.575 | 1.00 | 50.00 | C |
| ATOM | 1227 | C | VAL | B | 247 | −11.110 | 16.653 | 16.988 | 1.00 | 50.00 | C |
| ATOM | 1228 | O | VAL | B | 247 | −10.882 | 15.747 | 17.790 | 1.00 | 50.00 | O |
| ATOM | 1229 | CB | VAL | B | 247 | −11.330 | 14.969 | 14.894 | 1.00 | 50.00 | C |
| ATOM | 1230 | CG1 | VAL | B | 247 | −12.239 | 13.764 | 15.145 | 1.00 | 50.00 | C |
| ATOM | 1231 | CG2 | VAL | B | 247 | −11.048 | 14.996 | 13.393 | 1.00 | 50.00 | C |
| ATOM | 1232 | N | SER | B | 248 | −11.168 | 17.905 | 17.413 | 1.00 | 50.00 | N |
| ATOM | 1233 | CA | SER | B | 248 | −11.118 | 18.328 | 18.812 | 1.00 | 50.00 | C |
| ATOM | 1234 | C | SER | B | 248 | −12.315 | 19.175 | 19.227 | 1.00 | 50.00 | C |
| ATOM | 1235 | O | SER | B | 248 | −12.906 | 18.797 | 20.238 | 1.00 | 50.00 | O |
| ATOM | 1236 | CB | SER | B | 248 | −9.761 | 18.973 | 19.078 | 1.00 | 50.00 | C |
| ATOM | 1237 | OG | SER | B | 248 | −8.772 | 17.959 | 19.182 | 1.00 | 50.00 | O |
| ATOM | 1238 | N | THR | B | 249 | −12.732 | 20.228 | 18.539 | 1.00 | 50.00 | N |
| ATOM | 1239 | CA | THR | B | 249 | −13.921 | 21.009 | 18.890 | 1.00 | 50.00 | C |
| ATOM | 1240 | C | THR | B | 249 | −15.207 | 20.214 | 19.101 | 1.00 | 50.00 | C |
| ATOM | 1241 | O | THR | B | 249 | −15.817 | 20.396 | 20.153 | 1.00 | 50.00 | O |
| ATOM | 1242 | CB | THR | B | 249 | −14.114 | 22.237 | 17.996 | 1.00 | 50.00 | C |
| ATOM | 1243 | OG1 | THR | B | 249 | −13.017 | 23.128 | 18.135 | 1.00 | 50.00 | O |
| ATOM | 1244 | CG2 | THR | B | 249 | −15.362 | 23.072 | 18.281 | 1.00 | 50.00 | C |
| ATOM | 1245 | N | TYR | B | 250 | −15.651 | 19.366 | 18.187 | 1.00 | 50.00 | N |
| ATOM | 1246 | CA | TYR | B | 250 | −16.826 | 18.512 | 18.380 | 1.00 | 50.00 | C |
| ATOM | 1247 | C | TYR | B | 250 | −16.791 | 17.476 | 19.503 | 1.00 | 50.00 | C |
| ATOM | 1248 | O | TYR | B | 250 | −17.849 | 17.166 | 20.046 | 1.00 | 50.00 | O |
| ATOM | 1249 | CB | TYR | B | 250 | −17.302 | 18.062 | 16.997 | 1.00 | 50.00 | C |
| ATOM | 1250 | CG | TYR | B | 250 | −18.255 | 16.908 | 16.754 | 1.00 | 50.00 | C |
| ATOM | 1251 | CD1 | TYR | B | 250 | −19.199 | 17.097 | 15.808 | 1.00 | 50.00 | C |
| ATOM | 1252 | CD2 | TYR | B | 250 | −18.180 | 15.687 | 17.326 | 1.00 | 50.00 | C |
| ATOM | 1253 | CE1 | TYR | B | 250 | −20.222 | 16.228 | 15.695 | 1.00 | 50.00 | C |
| ATOM | 1254 | CE2 | TYR | B | 250 | −19.016 | 14.690 | 16.977 | 1.00 | 50.00 | C |
| ATOM | 1255 | CZ | TYR | B | 250 | −20.108 | 15.006 | 16.253 | 1.00 | 50.00 | C |
| ATOM | 1256 | OH | TYR | B | 250 | −21.163 | 14.137 | 16.174 | 1.00 | 50.00 | O |
| ATOM | 1257 | N | MET | B | 251 | −15.646 | 16.900 | 19.831 | 1.00 | 50.00 | N |
| ATOM | 1258 | CA | MET | B | 251 | −15.378 | 15.768 | 20.725 | 1.00 | 50.00 | C |
| ATOM | 1259 | C | MET | B | 251 | −15.409 | 16.288 | 22.156 | 1.00 | 50.00 | C |
| ATOM | 1260 | O | MET | B | 251 | −16.113 | 15.723 | 22.991 | 1.00 | 50.00 | O |
| ATOM | 1261 | CB | MET | B | 251 | −14.016 | 15.071 | 20.628 | 1.00 | 50.00 | C |
| ATOM | 1262 | CG | MET | B | 251 | −13.614 | 13.910 | 19.718 | 1.00 | 50.00 | C |
| ATOM | 1263 | SD | MET | B | 251 | −14.875 | 12.642 | 19.517 | 1.00 | 50.00 | S |
| ATOM | 1264 | CE | MET | B | 251 | −14.173 | 11.525 | 20.742 | 1.00 | 50.00 | C |
| ATOM | 1265 | N | LEU | B | 252 | −14.677 | 17.359 | 22.424 | 1.00 | 50.00 | N |
| ATOM | 1266 | CA | LEU | B | 252 | −15.078 | 18.377 | 23.392 | 1.00 | 50.00 | C |
| ATOM | 1267 | C | LEU | B | 252 | −16.575 | 18.617 | 23.554 | 1.00 | 50.00 | C |
| ATOM | 1268 | O | LEU | B | 252 | −17.108 | 18.340 | 24.627 | 1.00 | 50.00 | O |
| ATOM | 1269 | CB | LEU | B | 252 | −14.299 | 19.660 | 23.101 | 1.00 | 50.00 | C |
| ATOM | 1270 | CG | LEU | B | 252 | −14.647 | 20.851 | 23.992 | 1.00 | 50.00 | C |
| ATOM | 1271 | CD1 | LEU | B | 252 | −14.525 | 20.459 | 25.464 | 1.00 | 50.00 | C |
| ATOM | 1272 | CD2 | LEU | B | 252 | −13.756 | 22.035 | 23.631 | 1.00 | 50.00 | C |
| ATOM | 1273 | N | THR | B | 253 | −17.245 | 19.073 | 22.509 | 1.00 | 50.00 | N |
| ATOM | 1274 | CA | THR | B | 253 | −18.698 | 19.244 | 22.504 | 1.00 | 50.00 | C |
| ATOM | 1275 | C | THR | B | 253 | −19.556 | 17.985 | 22.599 | 1.00 | 50.00 | C |
| ATOM | 1276 | O | THR | B | 253 | −20.781 | 18.101 | 22.622 | 1.00 | 50.00 | O |
| ATOM | 1277 | CB | THR | B | 253 | −18.980 | 20.148 | 21.304 | 1.00 | 50.00 | C |
| ATOM | 1278 | OG1 | THR | B | 253 | −18.259 | 21.367 | 21.410 | 1.00 | 50.00 | O |
| ATOM | 1279 | CG2 | THR | B | 253 | −20.443 | 20.542 | 21.131 | 1.00 | 50.00 | C |
| ATOM | 1280 | N | ASN | B | 254 | −18.989 | 16.793 | 22.699 | 1.00 | 50.00 | N |
| ATOM | 1281 | CA | ASN | B | 254 | −19.685 | 15.506 | 22.741 | 1.00 | 50.00 | C |
| ATOM | 1282 | C | ASN | B | 254 | −20.740 | 15.285 | 21.665 | 1.00 | 50.00 | C |
| ATOM | 1283 | O | ASN | B | 254 | −21.901 | 14.992 | 21.952 | 1.00 | 50.00 | O |
| ATOM | 1284 | CB | ASN | B | 254 | −20.221 | 15.222 | 24.146 | 1.00 | 50.00 | C |
| ATOM | 1285 | CG | ASN | B | 254 | −19.228 | 14.918 | 25.258 | 1.00 | 50.00 | C |
| ATOM | 1286 | OD1 | ASN | B | 254 | −19.290 | 15.502 | 26.338 | 1.00 | 50.00 | O |
| ATOM | 1287 | ND2 | ASN | B | 254 | −18.330 | 13.971 | 25.050 | 1.00 | 50.00 | N |
| ATOM | 1288 | N | SER | B | 255 | −20.361 | 15.467 | 20.412 | 1.00 | 50.00 | N |
| ATOM | 1289 | CA | SER | B | 255 | −21.294 | 15.445 | 19.287 | 1.00 | 50.00 | C |
| ATOM | 1290 | C | SER | B | 255 | −22.315 | 16.549 | 19.043 | 1.00 | 50.00 | C |
| ATOM | 1291 | O | SER | B | 255 | −22.882 | 16.607 | 17.952 | 1.00 | 50.00 | O |
| ATOM | 1292 | CB | SER | B | 255 | −21.823 | 14.032 | 19.030 | 1.00 | 50.00 | C |
| ATOM | 1293 | OG | SER | B | 255 | −20.779 | 13.083 | 18.865 | 1.00 | 50.00 | O |
| ATOM | 1294 | N | GLU | B | 256 | −22.537 | 17.435 | 19.998 | 1.00 | 50.00 | N |
| ATOM | 1295 | CA | GLU | B | 256 | −23.741 | 18.263 | 20.063 | 1.00 | 50.00 | C |
| ATOM | 1296 | C | GLU | B | 256 | −23.419 | 19.733 | 19.822 | 1.00 | 50.00 | C |
| ATOM | 1297 | O | GLU | B | 256 | −23.359 | 20.523 | 20.763 | 1.00 | 50.00 | O |
| ATOM | 1298 | CB | GLU | B | 256 | −24.382 | 17.968 | 21.420 | 1.00 | 50.00 | C |
| ATOM | 1299 | CG | GLU | B | 256 | −25.097 | 16.624 | 21.558 | 1.00 | 50.00 | C |
| ATOM | 1300 | CD | GLU | B | 256 | −26.333 | 16.504 | 20.679 | 1.00 | 50.00 | C |
| ATOM | 1301 | OE1 | GLU | B | 256 | −26.219 | 15.990 | 19.545 | 1.00 | 50.00 | O |
| ATOM | 1302 | OE2 | GLU | B | 256 | −27.422 | 16.945 | 21.104 | 1.00 | 50.00 | O |
| ATOM | 1303 | N | LEU | B | 257 | −23.181 | 20.125 | 18.581 | 1.00 | 50.00 | N |
| ATOM | 1304 | CA | LEU | B | 257 | −22.585 | 21.419 | 18.235 | 1.00 | 50.00 | C |
| ATOM | 1305 | C | LEU | B | 257 | −23.420 | 22.664 | 18.489 | 1.00 | 50.00 | C |
| ATOM | 1306 | O | LEU | B | 257 | −22.932 | 23.675 | 18.992 | 1.00 | 50.00 | O |
| ATOM | 1307 | CB | LEU | B | 257 | −22.099 | 21.473 | 16.787 | 1.00 | 50.00 | C |
| ATOM | 1308 | CG | LEU | B | 257 | −20.703 | 20.889 | 16.582 | 1.00 | 50.00 | C |
| ATOM | 1309 | CD1 | LEU | B | 257 | −20.680 | 19.430 | 17.029 | 1.00 | 50.00 | C |
| ATOM | 1310 | CD2 | LEU | B | 257 | −20.304 | 21.069 | 15.120 | 1.00 | 50.00 | C |
| ATOM | 1311 | N | LEU | B | 258 | −24.688 | 22.521 | 18.148 | 1.00 | 50.00 | N |
| ATOM | 1312 | CA | LEU | B | 258 | −25.823 | 23.288 | 18.654 | 1.00 | 50.00 | C |
| ATOM | 1313 | C | LEU | B | 258 | −25.724 | 23.655 | 20.133 | 1.00 | 50.00 | C |
| ATOM | 1314 | O | LEU | B | 258 | −25.773 | 24.848 | 20.429 | 1.00 | 50.00 | O |
| ATOM | 1315 | CB | LEU | B | 258 | −27.056 | 22.473 | 18.259 | 1.00 | 50.00 | C |
| ATOM | 1316 | CG | LEU | B | 258 | −27.376 | 22.202 | 16.786 | 1.00 | 50.00 | C |
| ATOM | 1317 | CD1 | LEU | B | 258 | −26.463 | 21.232 | 16.036 | 1.00 | 50.00 | C |
| ATOM | 1318 | CD2 | LEU | B | 258 | −28.793 | 21.651 | 16.641 | 1.00 | 50.00 | C |
| ATOM | 1319 | N | SER | B | 259 | −25.440 | 22.721 | 21.028 | 1.00 | 50.00 | N |
| ATOM | 1320 | CA | SER | B | 259 | −25.041 | 22.988 | 22.411 | 1.00 | 50.00 | C |
| ATOM | 1321 | C | SER | B | 259 | −23.815 | 23.873 | 22.615 | 1.00 | 50.00 | C |
| ATOM | 1322 | O | SER | B | 259 | −24.006 | 24.933 | 23.210 | 1.00 | 50.00 | O |
| ATOM | 1323 | CB | SER | B | 259 | −24.931 | 21.709 | 23.241 | 1.00 | 50.00 | C |
| ATOM | 1324 | OG | SER | B | 259 | −26.111 | 20.921 | 23.175 | 1.00 | 50.00 | O |
| ATOM | 1325 | N | LEU | B | 260 | −22.617 | 23.534 | 22.161 | 1.00 | 50.00 | N |
| ATOM | 1326 | CA | LEU | B | 260 | −21.425 | 24.372 | 22.325 | 1.00 | 50.00 | C |
| ATOM | 1327 | C | LEU | B | 260 | −21.538 | 25.792 | 21.789 | 1.00 | 50.00 | C |
| ATOM | 1328 | O | LEU | B | 260 | −21.196 | 26.717 | 22.524 | 1.00 | 50.00 | O |
| ATOM | 1329 | CB | LEU | B | 260 | −20.124 | 23.794 | 21.766 | 1.00 | 50.00 | C |
| ATOM | 1330 | CG | LEU | B | 260 | −18.819 | 24.553 | 22.023 | 1.00 | 50.00 | C |
| ATOM | 1331 | CD1 | LEU | B | 260 | −18.429 | 24.395 | 23.492 | 1.00 | 50.00 | C |
| ATOM | 1332 | CD2 | LEU | B | 260 | −17.688 | 24.265 | 21.037 | 1.00 | 50.00 | C |
| ATOM | 1333 | N | ILE | B | 261 | −21.952 | 25.968 | 20.545 | 1.00 | 50.00 | N |
| ATOM | 1334 | CA | ILE | B | 261 | −22.195 | 27.277 | 19.941 | 1.00 | 50.00 | C |
| ATOM | 1335 | C | ILE | B | 261 | −23.135 | 28.102 | 20.814 | 1.00 | 50.00 | C |
| ATOM | 1336 | O | ILE | B | 261 | −22.665 | 29.117 | 21.322 | 1.00 | 50.00 | O |
| ATOM | 1337 | CB | ILE | B | 261 | −22.591 | 27.150 | 18.468 | 1.00 | 50.00 | C |
| ATOM | 1338 | CG1 | ILE | B | 261 | −21.590 | 26.427 | 17.564 | 1.00 | 50.00 | C |
| ATOM | 1339 | CG2 | ILE | B | 261 | −22.857 | 28.529 | 17.866 | 1.00 | 50.00 | C |
| ATOM | 1340 | CD1 | ILE | B | 261 | −22.204 | 25.958 | 16.247 | 1.00 | 50.00 | C |
| ATOM | 1341 | N | ASN | B | 262 | −24.350 | 27.688 | 21.134 | 1.00 | 50.00 | N |
| ATOM | 1342 | CA | ASN | B | 262 | −25.187 | 28.343 | 22.143 | 1.00 | 50.00 | C |
| ATOM | 1343 | C | ASN | B | 262 | −24.553 | 28.763 | 23.467 | 1.00 | 50.00 | C |
| ATOM | 1344 | O | ASN | B | 262 | −24.834 | 29.868 | 23.931 | 1.00 | 50.00 | O |
| ATOM | 1345 | CB | ASN | B | 262 | −26.478 | 27.543 | 22.324 | 1.00 | 50.00 | C |
| ATOM | 1346 | CG | ASN | B | 262 | −27.444 | 27.407 | 21.153 | 1.00 | 50.00 | C |
| ATOM | 1347 | OD1 | ASN | B | 262 | −28.026 | 26.345 | 20.938 | 1.00 | 50.00 | O |
| ATOM | 1348 | ND2 | ASN | B | 262 | −27.679 | 28.440 | 20.364 | 1.00 | 50.00 | N |
| ATOM | 1349 | N | ASP | B | 263 | −23.649 | 27.978 | 24.032 | 1.00 | 50.00 | N |
| ATOM | 1350 | CA | ASP | B | 263 | −22.771 | 28.361 | 25.140 | 1.00 | 50.00 | C |
| ATOM | 1351 | C | ASP | B | 263 | −21.718 | 29.426 | 24.844 | 1.00 | 50.00 | C |
| ATOM | 1352 | O | ASP | B | 263 | −21.632 | 30.402 | 25.588 | 1.00 | 50.00 | O |
| ATOM | 1353 | CB | ASP | B | 263 | −22.113 | 27.116 | 25.738 | 1.00 | 50.00 | C |
| ATOM | 1354 | CG | ASP | B | 263 | −23.036 | 26.075 | 26.355 | 1.00 | 50.00 | C |
| ATOM | 1355 | OD1 | ASP | B | 263 | −23.805 | 26.419 | 27.279 | 1.00 | 50.00 | O |
| ATOM | 1356 | OD2 | ASP | B | 263 | −23.004 | 24.904 | 25.920 | 1.00 | 50.00 | O |
| ATOM | 1357 | N | MET | B | 264 | −20.922 | 29.272 | 23.798 | 1.00 | 50.00 | N |
| ATOM | 1358 | CA | MET | B | 264 | −19.904 | 30.215 | 23.332 | 1.00 | 50.00 | C |
| ATOM | 1359 | C | MET | B | 264 | −20.595 | 31.486 | 22.837 | 1.00 | 50.00 | C |
| ATOM | 1360 | O | MET | B | 264 | −20.576 | 32.438 | 23.617 | 1.00 | 50.00 | O |
| ATOM | 1361 | CB | MET | B | 264 | −18.978 | 29.360 | 22.462 | 1.00 | 50.00 | C |
| ATOM | 1362 | CG | MET | B | 264 | −17.695 | 29.938 | 21.867 | 1.00 | 50.00 | C |
| ATOM | 1363 | SD | MET | B | 264 | −16.986 | 28.944 | 20.542 | 1.00 | 50.00 | S |
| ATOM | 1364 | CE | MET | B | 264 | −18.232 | 28.744 | 19.257 | 1.00 | 50.00 | C |
| ATOM | 1365 | N | PRO | B | 265 | −21.269 | 31.569 | 21.697 | 1.00 | 50.00 | N |
| ATOM | 1366 | CA | PRO | B | 265 | −22.221 | 32.641 | 21.382 | 1.00 | 50.00 | C |
| ATOM | 1367 | C | PRO | B | 265 | −23.719 | 32.343 | 21.278 | 1.00 | 50.00 | C |
| ATOM | 1368 | O | PRO | B | 265 | −24.106 | 31.235 | 20.911 | 1.00 | 50.00 | O |
| ATOM | 1369 | CB | PRO | B | 265 | −21.523 | 33.080 | 20.095 | 1.00 | 50.00 | C |
| ATOM | 1370 | CG | PRO | B | 265 | −21.157 | 31.821 | 19.319 | 1.00 | 50.00 | C |
| ATOM | 1371 | CD | PRO | B | 265 | −20.621 | 31.028 | 20.501 | 1.00 | 50.00 | C |
| ATOM | 1372 | N | ILE | B | 266 | −24.627 | 33.283 | 21.507 | 1.00 | 50.00 | N |
| ATOM | 1373 | CA | ILE | B | 266 | −26.052 | 33.281 | 21.147 | 1.00 | 50.00 | C |
| ATOM | 1374 | C | ILE | B | 266 | −26.355 | 33.136 | 19.651 | 1.00 | 50.00 | C |
| ATOM | 1375 | O | ILE | B | 266 | −25.464 | 32.994 | 18.814 | 1.00 | 50.00 | O |
| ATOM | 1376 | CB | ILE | B | 266 | −26.736 | 34.452 | 21.871 | 1.00 | 50.00 | C |
| ATOM | 1377 | CG1 | ILE | B | 266 | −26.534 | 34.610 | 23.382 | 1.00 | 50.00 | C |
| ATOM | 1378 | CG2 | ILE | B | 266 | −28.238 | 34.627 | 21.634 | 1.00 | 50.00 | C |
| ATOM | 1379 | CD1 | ILE | B | 266 | −26.848 | 35.962 | 24.022 | 1.00 | 50.00 | C |
| ATOM | 1380 | N | THR | B | 267 | −27.591 | 33.085 | 19.178 | 1.00 | 50.00 | N |
| ATOM | 1381 | CA | THR | B | 267 | −27.992 | 33.234 | 17.775 | 1.00 | 50.00 | C |
| ATOM | 1382 | C | THR | B | 267 | −27.780 | 34.603 | 17.136 | 1.00 | 50.00 | C |
| ATOM | 1383 | O | THR | B | 267 | −27.228 | 34.628 | 16.036 | 1.00 | 50.00 | O |
| ATOM | 1384 | CB | THR | B | 267 | −29.442 | 32.802 | 17.545 | 1.00 | 50.00 | C |
| ATOM | 1385 | OG1 | THR | B | 267 | −29.605 | 31.442 | 17.922 | 1.00 | 50.00 | O |
| ATOM | 1386 | CG2 | THR | B | 267 | −29.997 | 32.939 | 16.126 | 1.00 | 50.00 | C |
| ATOM | 1387 | N | ASN | B | 268 | −28.273 | 35.700 | 17.689 | 1.00 | 50.00 | N |
| ATOM | 1388 | CA | ASN | B | 268 | −27.998 | 37.062 | 17.222 | 1.00 | 50.00 | C |
| ATOM | 1389 | C | ASN | B | 268 | −26.571 | 37.418 | 16.818 | 1.00 | 50.00 | C |
| ATOM | 1390 | O | ASN | B | 268 | −26.347 | 38.177 | 15.876 | 1.00 | 50.00 | O |
| ATOM | 1391 | CB | ASN | B | 268 | −28.583 | 38.083 | 18.202 | 1.00 | 50.00 | C |
| ATOM | 1392 | CG | ASN | B | 268 | −28.125 | 38.239 | 19.647 | 1.00 | 50.00 | C |
| ATOM | 1393 | OD1 | ASN | B | 268 | −28.542 | 39.167 | 20.338 | 1.00 | 50.00 | O |
| ATOM | 1394 | ND2 | ASN | B | 268 | −27.268 | 37.393 | 20.188 | 1.00 | 50.00 | N |
| ATOM | 1395 | N | ASP | B | 269 | −25.617 | 36.840 | 17.528 | 1.00 | 50.00 | N |
| ATOM | 1396 | CA | ASP | B | 269 | −24.187 | 36.873 | 17.232 | 1.00 | 50.00 | C |
| ATOM | 1397 | C | ASP | B | 269 | −23.703 | 36.002 | 16.076 | 1.00 | 50.00 | C |
| ATOM | 1398 | O | ASP | B | 269 | −22.560 | 36.113 | 15.639 | 1.00 | 50.00 | O |
| ATOM | 1399 | CB | ASP | B | 269 | −23.462 | 36.800 | 18.579 | 1.00 | 50.00 | C |
| ATOM | 1400 | CG | ASP | B | 269 | −23.875 | 35.921 | 19.741 | 1.00 | 50.00 | C |
| ATOM | 1401 | OD1 | ASP | B | 269 | −24.178 | 36.338 | 20.880 | 1.00 | 50.00 | O |
| ATOM | 1402 | OD2 | ASP | B | 269 | −23.866 | 34.721 | 19.405 | 1.00 | 50.00 | O |
| ATOM | 1403 | N | GLN | B | 270 | −24.527 | 35.238 | 15.380 | 1.00 | 50.00 | N |
| ATOM | 1404 | CA | GLN | B | 270 | −24.167 | 34.583 | 14.122 | 1.00 | 50.00 | C |
| ATOM | 1405 | C | GLN | B | 270 | −24.291 | 35.487 | 12.896 | 1.00 | 50.00 | C |
| ATOM | 1406 | O | GLN | B | 270 | −24.810 | 36.598 | 13.001 | 1.00 | 50.00 | O |
| ATOM | 1407 | CB | GLN | B | 270 | −24.866 | 33.224 | 14.069 | 1.00 | 50.00 | C |
| ATOM | 1408 | CG | GLN | B | 270 | −24.811 | 32.302 | 15.287 | 1.00 | 50.00 | C |
| ATOM | 1409 | CD | GLN | B | 270 | −23.489 | 31.643 | 15.653 | 1.00 | 50.00 | C |
| ATOM | 1410 | OE1 | GLN | B | 270 | −22.639 | 31.272 | 14.845 | 1.00 | 50.00 | O |
| ATOM | 1411 | NE2 | GLN | B | 270 | −23.338 | 31.377 | 16.935 | 1.00 | 50.00 | N |
| ATOM | 1412 | N | LYS | B | 271 | −23.743 | 35.065 | 11.765 | 1.00 | 50.00 | N |
| ATOM | 1413 | CA | LYS | B | 271 | −23.549 | 35.727 | 10.469 | 1.00 | 50.00 | C |
| ATOM | 1414 | C | LYS | B | 271 | −23.454 | 35.107 | 9.051 | 1.00 | 50.00 | C |
| ATOM | 1415 | O | LYS | B | 271 | −24.136 | 35.796 | 8.293 | 1.00 | 50.00 | O |
| ATOM | 1416 | CB | LYS | B | 271 | −22.985 | 37.123 | 10.782 | 1.00 | 50.00 | C |
| ATOM | 1417 | CG | LYS | B | 271 | −21.888 | 37.680 | 11.702 | 1.00 | 50.00 | C |
| ATOM | 1418 | CD | LYS | B | 271 | −21.893 | 38.218 | 13.140 | 1.00 | 50.00 | C |
| ATOM | 1419 | CE | LYS | B | 271 | −21.739 | 39.573 | 13.840 | 1.00 | 50.00 | C |
| ATOM | 1420 | NZ | LYS | B | 271 | −22.768 | 40.556 | 14.211 | 1.00 | 50.00 | N |
| ATOM | 1421 | N | LYS | B | 272 | −23.087 | 34.159 | 8.178 | 1.00 | 50.00 | N |
| ATOM | 1422 | CA | LYS | B | 272 | −22.087 | 33.161 | 7.737 | 1.00 | 50.00 | C |
| ATOM | 1423 | C | LYS | B | 272 | −21.842 | 31.672 | 8.049 | 1.00 | 50.00 | C |
| ATOM | 1424 | O | LYS | B | 272 | −22.118 | 30.948 | 7.093 | 1.00 | 50.00 | O |
| ATOM | 1425 | CB | LYS | B | 272 | −20.970 | 33.564 | 6.760 | 1.00 | 50.00 | C |
| ATOM | 1426 | CG | LYS | B | 272 | −19.895 | 32.649 | 6.152 | 1.00 | 50.00 | C |
| ATOM | 1427 | CD | LYS | B | 272 | −19.574 | 32.602 | 4.657 | 1.00 | 50.00 | C |
| ATOM | 1428 | CE | LYS | B | 272 | −18.783 | 31.391 | 4.159 | 1.00 | 50.00 | C |
| ATOM | 1429 | NZ | LYS | B | 272 | −18.258 | 31.566 | 2.796 | 1.00 | 50.00 | N |
| ATOM | 1430 | N | LEU | B | 273 | −21.319 | 30.821 | 8.931 | 1.00 | 50.00 | N |
| ATOM | 1431 | CA | LEU | B | 273 | −20.415 | 30.903 | 10.088 | 1.00 | 50.00 | C |
| ATOM | 1432 | C | LEU | B | 273 | −20.699 | 30.055 | 11.330 | 1.00 | 50.00 | C |
| ATOM | 1433 | O | LEU | B | 273 | −21.280 | 30.488 | 12.324 | 1.00 | 50.00 | O |
| ATOM | 1434 | CB | LEU | B | 273 | −19.936 | 32.250 | 10.632 | 1.00 | 50.00 | C |
| ATOM | 1435 | CG | LEU | B | 273 | −21.021 | 33.321 | 10.681 | 1.00 | 50.00 | C |
| ATOM | 1436 | CD1 | LEU | B | 273 | −22.415 | 32.762 | 10.995 | 1.00 | 50.00 | C |
| ATOM | 1437 | CD2 | LEU | B | 273 | −20.536 | 34.721 | 10.303 | 1.00 | 50.00 | C |
| ATOM | 1438 | N | MET | B | 274 | −20.179 | 28.849 | 11.484 | 1.00 | 50.00 | N |
| ATOM | 1439 | CA | MET | B | 274 | −19.067 | 28.586 | 12.405 | 1.00 | 50.00 | C |
| ATOM | 1440 | C | MET | B | 274 | −17.678 | 28.941 | 11.875 | 1.00 | 50.00 | C |
| ATOM | 1441 | O | MET | B | 274 | −16.722 | 28.970 | 12.648 | 1.00 | 50.00 | O |
| ATOM | 1442 | CB | MET | B | 274 | −19.115 | 27.118 | 12.837 | 1.00 | 50.00 | C |
| ATOM | 1443 | CG | MET | B | 274 | −19.637 | 26.678 | 14.202 | 1.00 | 50.00 | C |
| ATOM | 1444 | SD | MET | B | 274 | −18.314 | 26.522 | 15.414 | 1.00 | 50.00 | S |
| ATOM | 1445 | CE | MET | B | 274 | −18.238 | 24.739 | 15.665 | 1.00 | 50.00 | C |
| ATOM | 1446 | N | SER | B | 275 | −17.542 | 29.199 | 10.584 | 1.00 | 50.00 | N |
| ATOM | 1447 | CA | SER | B | 275 | −16.326 | 29.387 | 9.792 | 1.00 | 50.00 | C |
| ATOM | 1448 | C | SER | B | 275 | −15.427 | 30.535 | 10.221 | 1.00 | 50.00 | C |
| ATOM | 1449 | O | SER | B | 275 | −14.217 | 30.368 | 10.368 | 1.00 | 50.00 | O |
| ATOM | 1450 | CB | SER | B | 275 | −16.659 | 29.622 | 8.317 | 1.00 | 50.00 | C |
| ATOM | 1451 | OG | SER | B | 275 | −17.416 | 30.796 | 8.054 | 1.00 | 50.00 | O |
| ATOM | 1452 | N | ASN | B | 276 | −16.057 | 31.680 | 10.409 | 1.00 | 50.00 | N |
| ATOM | 1453 | CA | ASN | B | 276 | −15.452 | 32.887 | 10.966 | 1.00 | 50.00 | C |
| ATOM | 1454 | C | ASN | B | 276 | −14.875 | 32.593 | 12.342 | 1.00 | 50.00 | C |
| ATOM | 1455 | O | ASN | B | 276 | −13.648 | 32.585 | 12.416 | 1.00 | 50.00 | O |
| ATOM | 1456 | CB | ASN | B | 276 | −16.495 | 34.000 | 10.890 | 1.00 | 50.00 | C |
| ATOM | 1457 | CG | ASN | B | 276 | −16.877 | 34.343 | 9.457 | 1.00 | 50.00 | C |
| ATOM | 1458 | OD1 | ASN | B | 276 | −18.019 | 34.171 | 9.040 | 1.00 | 50.00 | O |
| ATOM | 1459 | ND2 | ASN | B | 276 | −15.983 | 34.663 | 8.542 | 1.00 | 50.00 | N |
| ATOM | 1460 | N | ASN | B | 277 | −15.664 | 32.150 | 13.305 | 1.00 | 50.00 | N |
| ATOM | 1461 | CA | ASN | B | 277 | −15.188 | 31.553 | 14.554 | 1.00 | 50.00 | C |
| ATOM | 1462 | C | ASN | B | 277 | −13.957 | 30.657 | 14.475 | 1.00 | 50.00 | C |
| ATOM | 1463 | O | ASN | B | 277 | −12.929 | 31.087 | 14.995 | 1.00 | 50.00 | O |
| ATOM | 1464 | CB | ASN | B | 277 | −16.379 | 31.020 | 15.349 | 1.00 | 50.00 | C |
| ATOM | 1465 | CG | ASN | B | 277 | −17.500 | 31.989 | 15.704 | 1.00 | 50.00 | C |
| ATOM | 1466 | OD1 | ASN | B | 277 | −18.647 | 31.569 | 15.846 | 1.00 | 50.00 | O |
| ATOM | 1467 | ND2 | ASN | B | 277 | −17.220 | 33.262 | 15.929 | 1.00 | 50.00 | N |
| ATOM | 1468 | N | VAL | B | 278 | −13.979 | 29.568 | 13.722 | 1.00 | 50.00 | N |
| ATOM | 1469 | CA | VAL | B | 278 | −12.815 | 28.761 | 13.346 | 1.00 | 50.00 | C |
| ATOM | 1470 | C | VAL | B | 278 | −11.598 | 29.539 | 12.854 | 1.00 | 50.00 | C |
| ATOM | 1471 | O | VAL | B | 278 | −10.516 | 29.432 | 13.431 | 1.00 | 50.00 | O |
| ATOM | 1472 | CB | VAL | B | 278 | −13.237 | 27.631 | 12.402 | 1.00 | 50.00 | C |
| ATOM | 1473 | CG1 | VAL | B | 278 | −12.091 | 26.801 | 11.822 | 1.00 | 50.00 | C |
| ATOM | 1474 | CG2 | VAL | B | 278 | −14.231 | 26.646 | 13.018 | 1.00 | 50.00 | C |
| ATOM | 1475 | N | GLN | B | 279 | −11.741 | 30.300 | 11.782 | 1.00 | 50.00 | N |
| ATOM | 1476 | CA | GLN | B | 279 | −10.684 | 31.137 | 11.214 | 1.00 | 50.00 | C |
| ATOM | 1477 | C | GLN | B | 279 | −10.166 | 32.304 | 12.048 | 1.00 | 50.00 | C |
| ATOM | 1478 | O | GLN | B | 279 | −9.039 | 32.728 | 11.792 | 1.00 | 50.00 | O |
| ATOM | 1479 | CB | GLN | B | 279 | −11.151 | 31.600 | 9.834 | 1.00 | 50.00 | C |
| ATOM | 1480 | CG | GLN | B | 279 | −11.237 | 30.557 | 8.721 | 1.00 | 50.00 | C |
| ATOM | 1481 | CD | GLN | B | 279 | −9.906 | 29.898 | 8.390 | 1.00 | 50.00 | C |
| ATOM | 1482 | OE1 | GLN | B | 279 | −8.825 | 30.477 | 8.481 | 1.00 | 50.00 | O |
| ATOM | 1483 | NE2 | GLN | B | 279 | −9.965 | 28.637 | 8.007 | 1.00 | 50.00 | N |
| ATOM | 1484 | N | ILE | B | 280 | −10.902 | 32.818 | 13.020 | 1.00 | 50.00 | N |
| ATOM | 1485 | CA | ILE | B | 280 | −10.471 | 33.858 | 13.959 | 1.00 | 50.00 | C |
| ATOM | 1486 | C | ILE | B | 280 | −9.184 | 33.555 | 14.714 | 1.00 | 50.00 | C |
| ATOM | 1487 | O | ILE | B | 280 | −8.488 | 34.521 | 15.021 | 1.00 | 50.00 | O |
| ATOM | 1488 | CB | ILE | B | 280 | −11.593 | 34.291 | 14.907 | 1.00 | 50.00 | C |
| ATOM | 1489 | CG1 | ILE | B | 280 | −12.636 | 35.040 | 14.080 | 1.00 | 50.00 | C |
| ATOM | 1490 | CG2 | ILE | B | 280 | −11.238 | 35.231 | 16.059 | 1.00 | 50.00 | C |
| ATOM | 1491 | CD1 | ILE | B | 280 | −14.069 | 35.182 | 14.571 | 1.00 | 50.00 | C |
| ATOM | 1492 | N | VAL | B | 281 | −8.900 | 32.302 | 15.029 | 1.00 | 50.00 | N |
| ATOM | 1493 | CA | VAL | B | 281 | −7.777 | 31.903 | 15.881 | 1.00 | 50.00 | C |
| ATOM | 1494 | C | VAL | B | 281 | −8.046 | 32.102 | 17.372 | 1.00 | 50.00 | C |
| ATOM | 1495 | O | VAL | B | 281 | −7.135 | 32.003 | 18.193 | 1.00 | 50.00 | O |
| ATOM | 1496 | CB | VAL | B | 281 | −6.460 | 32.348 | 15.231 | 1.00 | 50.00 | C |
| ATOM | 1497 | CG1 | VAL | B | 281 | −5.168 | 32.264 | 16.041 | 1.00 | 50.00 | C |
| ATOM | 1498 | CG2 | VAL | B | 281 | −6.222 | 31.746 | 13.845 | 1.00 | 50.00 | C |
| ATOM | 1499 | N | ARG | B | 282 | −9.286 | 32.292 | 17.796 | 1.00 | 50.00 | N |
| ATOM | 1500 | CA | ARG | B | 282 | −9.736 | 32.412 | 19.183 | 1.00 | 50.00 | C |
| ATOM | 1501 | C | ARG | B | 282 | −9.778 | 31.126 | 20.004 | 1.00 | 50.00 | C |
| ATOM | 1502 | O | ARG | B | 282 | −9.354 | 31.129 | 21.159 | 1.00 | 50.00 | O |
| ATOM | 1503 | CB | ARG | B | 282 | −11.085 | 33.131 | 19.130 | 1.00 | 50.00 | C |
| ATOM | 1504 | CG | ARG | B | 282 | −12.220 | 32.363 | 18.451 | 1.00 | 50.00 | C |
| ATOM | 1505 | CD | ARG | B | 282 | −13.561 | 33.088 | 18.394 | 1.00 | 50.00 | C |
| ATOM | 1506 | NE | ARG | B | 282 | −14.709 | 32.184 | 18.325 | 1.00 | 50.00 | N |
| ATOM | 1507 | CZ | ARG | B | 282 | −15.924 | 32.416 | 18.795 | 1.00 | 50.00 | C |
| ATOM | 1508 | NH1 | ARG | B | 282 | −16.340 | 33.522 | 19.385 | 1.00 | 50.00 | N |
| ATOM | 1509 | NH2 | ARG | B | 282 | −16.821 | 31.457 | 18.680 | 1.00 | 50.00 | N |
| ATOM | 1510 | N | GLN | B | 283 | −10.294 | 30.049 | 19.434 | 1.00 | 50.00 | N |
| ATOM | 1511 | CA | GLN | B | 283 | −10.441 | 28.726 | 20.041 | 1.00 | 50.00 | C |
| ATOM | 1512 | C | GLN | B | 283 | −9.117 | 27.980 | 19.927 | 1.00 | 50.00 | C |
| ATOM | 1513 | O | GLN | B | 283 | −8.830 | 27.340 | 18.915 | 1.00 | 50.00 | O |
| ATOM | 1514 | CB | GLN | B | 283 | −11.623 | 27.961 | 19.440 | 1.00 | 50.00 | C |
| ATOM | 1515 | CG | GLN | B | 283 | −11.701 | 27.683 | 17.938 | 1.00 | 50.00 | C |
| ATOM | 1516 | CD | GLN | B | 283 | −12.018 | 28.917 | 17.107 | 1.00 | 50.00 | C |
| ATOM | 1517 | OE1 | GLN | B | 283 | −13.090 | 29.505 | 17.239 | 1.00 | 50.00 | O |
| ATOM | 1518 | NE2 | GLN | B | 283 | −11.078 | 29.358 | 16.291 | 1.00 | 50.00 | N |
| ATOM | 1519 | N | GLN | B | 284 | −8.284 | 28.111 | 20.945 | 1.00 | 50.00 | N |
| ATOM | 1520 | CA | GLN | B | 284 | −6.925 | 27.568 | 20.937 | 1.00 | 50.00 | C |
| ATOM | 1521 | C | GLN | B | 284 | −6.702 | 26.629 | 22.114 | 1.00 | 50.00 | C |
| ATOM | 1522 | O | GLN | B | 284 | −7.312 | 26.763 | 23.174 | 1.00 | 50.00 | O |
| ATOM | 1523 | CB | GLN | B | 284 | −5.875 | 28.679 | 20.891 | 1.00 | 50.00 | C |
| ATOM | 1524 | CG | GLN | B | 284 | −5.723 | 29.351 | 19.527 | 1.00 | 50.00 | C |
| ATOM | 1525 | CD | GLN | B | 284 | −5.234 | 28.461 | 18.392 | 1.00 | 50.00 | C |
| ATOM | 1526 | OE1 | GLN | B | 284 | −4.719 | 27.358 | 18.570 | 1.00 | 50.00 | O |
| ATOM | 1527 | NE2 | GLN | B | 284 | −5.390 | 28.917 | 17.163 | 1.00 | 50.00 | N |
| ATOM | 1528 | N | SER | B | 285 | −5.812 | 25.675 | 21.893 | 1.00 | 50.00 | N |
| ATOM | 1529 | CA | SER | B | 285 | −5.548 | 24.581 | 22.827 | 1.00 | 50.00 | C |
| ATOM | 1530 | C | SER | B | 285 | −4.057 | 24.291 | 22.934 | 1.00 | 50.00 | C |
| ATOM | 1531 | O | SER | B | 285 | −3.294 | 24.496 | 21.990 | 1.00 | 50.00 | O |
| ATOM | 1532 | CB | SER | B | 285 | −6.288 | 23.310 | 22.413 | 1.00 | 50.00 | C |
| ATOM | 1533 | OG | SER | B | 285 | −7.671 | 23.530 | 22.176 | 1.00 | 50.00 | O |
| ATOM | 1534 | N | TYR | B | 286 | −3.646 | 23.801 | 24.093 | 1.00 | 50.00 | N |
| ATOM | 1535 | CA | TYR | B | 286 | −2.239 | 23.540 | 24.397 | 1.00 | 50.00 | C |
| ATOM | 1536 | C | TYR | B | 286 | −2.135 | 22.323 | 25.304 | 1.00 | 50.00 | C |
| ATOM | 1537 | O | TYR | B | 286 | −2.819 | 22.243 | 26.324 | 1.00 | 50.00 | O |
| ATOM | 1538 | CB | TYR | B | 286 | −1.555 | 24.741 | 25.054 | 1.00 | 50.00 | C |
| ATOM | 1539 | CG | TYR | B | 286 | −1.293 | 25.887 | 24.101 | 1.00 | 50.00 | C |
| ATOM | 1540 | CD1 | TYR | B | 286 | −0.373 | 25.722 | 23.128 | 1.00 | 50.00 | C |
| ATOM | 1541 | CD2 | TYR | B | 286 | −2.088 | 26.978 | 24.090 | 1.00 | 50.00 | C |
| ATOM | 1542 | CE1 | TYR | B | 286 | −0.284 | 26.615 | 22.123 | 1.00 | 50.00 | C |
| ATOM | 1543 | CE2 | TYR | B | 286 | −1.977 | 27.889 | 23.102 | 1.00 | 50.00 | C |
| ATOM | 1544 | CZ | TYR | B | 286 | −1.071 | 27.710 | 22.119 | 1.00 | 50.00 | C |
| ATOM | 1545 | OH | TYR | B | 286 | −0.946 | 28.643 | 21.124 | 1.00 | 50.00 | O |
| ATOM | 1546 | N | SER | B | 287 | −1.176 | 21.456 | 25.024 | 1.00 | 50.00 | N |
| ATOM | 1547 | CA | SER | B | 287 | −0.734 | 20.421 | 25.960 | 1.00 | 50.00 | C |
| ATOM | 1548 | C | SER | B | 287 | −0.346 | 20.999 | 27.317 | 1.00 | 50.00 | C |
| ATOM | 1549 | O | SER | B | 287 | 0.214 | 22.091 | 27.404 | 1.00 | 50.00 | O |
| ATOM | 1550 | CB | SER | B | 287 | 0.406 | 19.620 | 25.331 | 1.00 | 50.00 | C |
| ATOM | 1551 | OG | SER | B | 287 | −0.039 | 18.885 | 24.200 | 1.00 | 50.00 | O |
| ATOM | 1552 | N | ILE | B | 288 | −0.740 | 20.303 | 28.369 | 1.00 | 50.00 | N |
| ATOM | 1553 | CA | ILE | B | 288 | −0.408 | 20.589 | 29.768 | 1.00 | 50.00 | C |
| ATOM | 1554 | C | ILE | B | 288 | 0.218 | 19.407 | 30.505 | 1.00 | 50.00 | C |
| ATOM | 1555 | O | ILE | B | 288 | 0.946 | 19.638 | 31.470 | 1.00 | 50.00 | O |
| ATOM | 1556 | CB | ILE | B | 288 | −1.561 | 21.200 | 30.567 | 1.00 | 50.00 | C |
| ATOM | 1557 | CG1 | ILE | B | 288 | −2.829 | 20.344 | 30.562 | 1.00 | 50.00 | C |
| ATOM | 1558 | CG2 | ILE | B | 288 | −1.820 | 22.656 | 30.179 | 1.00 | 50.00 | C |
| ATOM | 1559 | CD1 | ILE | B | 288 | −3.764 | 20.485 | 31.761 | 1.00 | 50.00 | C |
| ATOM | 1560 | N | MET | B | 289 | −0.093 | 18.170 | 30.151 | 1.00 | 50.00 | N |
| ATOM | 1561 | CA | MET | B | 289 | 0.232 | 16.966 | 30.915 | 1.00 | 50.00 | C |
| ATOM | 1562 | C | MET | B | 289 | 0.535 | 15.855 | 29.919 | 1.00 | 50.00 | C |
| ATOM | 1563 | O | MET | B | 289 | −0.195 | 15.672 | 28.946 | 1.00 | 50.00 | O |
| ATOM | 1564 | CB | MET | B | 289 | −0.923 | 16.482 | 31.792 | 1.00 | 50.00 | C |
| ATOM | 1565 | CG | MET | B | 289 | −1.360 | 17.407 | 32.925 | 1.00 | 50.00 | C |
| ATOM | 1566 | SD | MET | B | 289 | −0.044 | 17.553 | 34.145 | 1.00 | 50.00 | S |
| ATOM | 1567 | CE | MET | B | 289 | −0.118 | 15.937 | 34.937 | 1.00 | 50.00 | C |
| ATOM | 1568 | N | SER | B | 290 | 1.566 | 15.080 | 30.207 | 1.00 | 50.00 | N |
| ATOM | 1569 | CA | SER | B | 290 | 1.927 | 13.888 | 29.440 | 1.00 | 50.00 | C |
| ATOM | 1570 | C | SER | B | 290 | 2.332 | 12.834 | 30.463 | 1.00 | 50.00 | C |
| ATOM | 1571 | O | SER | B | 290 | 3.472 | 12.844 | 30.929 | 1.00 | 50.00 | O |
| ATOM | 1572 | CB | SER | B | 290 | 3.078 | 14.188 | 28.479 | 1.00 | 50.00 | C |
| ATOM | 1573 | OG | SER | B | 290 | 2.754 | 15.041 | 27.392 | 1.00 | 50.00 | O |
| ATOM | 1574 | N | ILE | B | 291 | 1.451 | 11.905 | 30.804 | 1.00 | 50.00 | N |
| ATOM | 1575 | CA | ILE | B | 291 | 1.788 | 10.738 | 31.621 | 1.00 | 50.00 | C |
| ATOM | 1576 | C | ILE | B | 291 | 2.474 | 9.756 | 30.677 | 1.00 | 50.00 | C |
| ATOM | 1577 | O | ILE | B | 291 | 1.817 | 9.100 | 29.869 | 1.00 | 50.00 | O |
| ATOM | 1578 | CB | ILE | B | 291 | 0.607 | 10.131 | 32.387 | 1.00 | 50.00 | C |
| ATOM | 1579 | CG1 | ILE | B | 291 | −0.178 | 11.099 | 33.274 | 1.00 | 50.00 | C |
| ATOM | 1580 | CG2 | ILE | B | 291 | 1.014 | 8.918 | 33.225 | 1.00 | 50.00 | C |
| ATOM | 1581 | CD1 | ILE | B | 291 | −1.577 | 10.657 | 33.703 | 1.00 | 50.00 | C |
| ATOM | 1582 | N | ILE | B | 292 | 3.788 | 9.644 | 30.788 | 1.00 | 50.00 | N |
| ATOM | 1583 | CA | ILE | B | 292 | 4.614 | 8.682 | 30.054 | 1.00 | 50.00 | C |
| ATOM | 1584 | C | ILE | B | 292 | 4.193 | 7.227 | 30.246 | 1.00 | 50.00 | C |
| ATOM | 1585 | O | ILE | B | 292 | 4.121 | 6.483 | 29.268 | 1.00 | 50.00 | O |
| ATOM | 1586 | CB | ILE | B | 292 | 6.098 | 8.978 | 30.306 | 1.00 | 50.00 | C |
| ATOM | 1587 | CG1 | ILE | B | 292 | 6.561 | 10.299 | 29.687 | 1.00 | 50.00 | C |
| ATOM | 1588 | CG2 | ILE | B | 292 | 7.028 | 7.840 | 29.883 | 1.00 | 50.00 | C |
| ATOM | 1589 | CD1 | ILE | B | 292 | 7.961 | 10.797 | 30.043 | 1.00 | 50.00 | C |
| ATOM | 1590 | N | LYS | B | 293 | 3.967 | 6.796 | 31.477 | 1.00 | 50.00 | N |
| ATOM | 1591 | CA | LYS | B | 293 | 3.700 | 5.405 | 31.844 | 1.00 | 50.00 | C |
| ATOM | 1592 | C | LYS | B | 293 | 2.331 | 4.881 | 31.423 | 1.00 | 50.00 | C |
| ATOM | 1593 | O | LYS | B | 293 | 2.261 | 3.903 | 30.680 | 1.00 | 50.00 | O |
| ATOM | 1594 | CB | LYS | B | 293 | 3.912 | 5.262 | 33.351 | 1.00 | 50.00 | C |
| ATOM | 1595 | CG | LYS | B | 293 | 5.354 | 5.446 | 33.821 | 1.00 | 50.00 | C |
| ATOM | 1596 | CD | LYS | B | 293 | 5.485 | 5.250 | 35.329 | 1.00 | 50.00 | C |
| ATOM | 1597 | CE | LYS | B | 293 | 6.918 | 5.487 | 35.801 | 1.00 | 50.00 | C |
| ATOM | 1598 | NZ | LYS | B | 293 | 7.031 | 5.217 | 37.242 | 1.00 | 50.00 | N |
| ATOM | 1599 | N | GLU | B | 294 | 1.262 | 5.526 | 31.862 | 1.00 | 50.00 | N |
| ATOM | 1600 | CA | GLU | B | 294 | −0.100 | 5.281 | 31.385 | 1.00 | 50.00 | C |
| ATOM | 1601 | C | GLU | B | 294 | −0.400 | 5.618 | 29.929 | 1.00 | 50.00 | C |
| ATOM | 1602 | O | GLU | B | 294 | −1.395 | 5.115 | 29.408 | 1.00 | 50.00 | O |
| ATOM | 1603 | CB | GLU | B | 294 | −1.094 | 6.020 | 32.282 | 1.00 | 50.00 | C |
| ATOM | 1604 | CG | GLU | B | 294 | −1.148 | 5.601 | 33.750 | 1.00 | 50.00 | C |
| ATOM | 1605 | CD | GLU | B | 294 | −1.724 | 4.207 | 33.948 | 1.00 | 50.00 | C |
| ATOM | 1606 | OE1 | GLU | B | 294 | −0.958 | 3.221 | 33.878 | 1.00 | 50.00 | O |
| ATOM | 1607 | OE2 | GLU | B | 294 | −2.951 | 4.083 | 34.149 | 1.00 | 50.00 | O |
| ATOM | 1608 | N | GLU | B | 295 | 0.394 | 6.454 | 29.278 | 1.00 | 50.00 | N |
| ATOM | 1609 | CA | GLU | B | 295 | 0.191 | 6.904 | 27.901 | 1.00 | 50.00 | C |
| ATOM | 1610 | C | GLU | B | 295 | −1.059 | 7.772 | 27.806 | 1.00 | 50.00 | C |
| ATOM | 1611 | O | GLU | B | 295 | −2.082 | 7.350 | 27.268 | 1.00 | 50.00 | O |
| ATOM | 1612 | CB | GLU | B | 295 | 0.359 | 5.754 | 26.904 | 1.00 | 50.00 | C |
| ATOM | 1613 | CG | GLU | B | 295 | 1.699 | 5.019 | 26.873 | 1.00 | 50.00 | C |
| ATOM | 1614 | CD | GLU | B | 295 | 2.763 | 5.819 | 26.137 | 1.00 | 50.00 | C |
| ATOM | 1615 | OE1 | GLU | B | 295 | 3.148 | 6.884 | 26.665 | 1.00 | 50.00 | O |
| ATOM | 1616 | OE2 | GLU | B | 295 | 3.186 | 5.425 | 25.029 | 1.00 | 50.00 | O |
| ATOM | 1617 | N | VAL | B | 296 | −0.984 | 8.960 | 28.384 | 1.00 | 50.00 | N |
| ATOM | 1618 | CA | VAL | B | 296 | −2.101 | 9.890 | 28.554 | 1.00 | 50.00 | C |
| ATOM | 1619 | C | VAL | B | 296 | −1.574 | 11.270 | 28.178 | 1.00 | 50.00 | C |
| ATOM | 1620 | O | VAL | B | 296 | −0.450 | 11.644 | 28.510 | 1.00 | 50.00 | O |
| ATOM | 1621 | CB | VAL | B | 296 | −2.685 | 9.870 | 29.970 | 1.00 | 50.00 | C |
| ATOM | 1622 | CG1 | VAL | B | 296 | −3.742 | 10.930 | 30.283 | 1.00 | 50.00 | C |
| ATOM | 1623 | CG2 | VAL | B | 296 | −3.229 | 8.500 | 30.372 | 1.00 | 50.00 | C |
| ATOM | 1624 | N | LEU | B | 297 | −2.431 | 12.070 | 27.565 | 1.00 | 50.00 | N |
| ATOM | 1625 | CA | LEU | B | 297 | −2.182 | 13.493 | 27.337 | 1.00 | 50.00 | C |
| ATOM | 1626 | C | LEU | B | 297 | −3.388 | 14.325 | 27.756 | 1.00 | 50.00 | C |
| ATOM | 1627 | O | LEU | B | 297 | −4.472 | 14.122 | 27.211 | 1.00 | 50.00 | O |
| ATOM | 1628 | CB | LEU | B | 297 | −1.855 | 13.679 | 25.855 | 1.00 | 50.00 | C |
| ATOM | 1629 | CG | LEU | B | 297 | −1.080 | 14.930 | 25.442 | 1.00 | 50.00 | C |
| ATOM | 1630 | CD1 | LEU | B | 297 | −1.708 | 16.250 | 25.882 | 1.00 | 50.00 | C |
| ATOM | 1631 | CD2 | LEU | B | 297 | 0.406 | 14.970 | 25.776 | 1.00 | 50.00 | C |
| ATOM | 1632 | N | ALA | B | 298 | −3.220 | 15.338 | 28.590 | 1.00 | 50.00 | N |
| ATOM | 1633 | CA | ALA | B | 298 | −4.209 | 16.405 | 28.755 | 1.00 | 50.00 | C |
| ATOM | 1634 | C | ALA | B | 298 | −3.909 | 17.638 | 27.909 | 1.00 | 50.00 | C |
| ATOM | 1635 | O | ALA | B | 298 | −2.885 | 18.287 | 28.117 | 1.00 | 50.00 | O |
| ATOM | 1636 | CB | ALA | B | 298 | −4.281 | 16.794 | 30.230 | 1.00 | 50.00 | C |
| ATOM | 1637 | N | TYR | B | 299 | −4.793 | 18.006 | 26.995 | 1.00 | 50.00 | N |
| ATOM | 1638 | CA | TYR | B | 299 | −4.857 | 19.348 | 26.411 | 1.00 | 50.00 | C |
| ATOM | 1639 | C | TYR | B | 299 | −5.483 | 20.358 | 27.369 | 1.00 | 50.00 | C |
| ATOM | 1640 | O | TYR | B | 299 | −6.115 | 19.974 | 28.353 | 1.00 | 50.00 | O |
| ATOM | 1641 | CB | TYR | B | 299 | −5.430 | 19.278 | 24.990 | 1.00 | 50.00 | C |
| ATOM | 1642 | CG | TYR | B | 299 | −6.778 | 19.768 | 24.505 | 1.00 | 50.00 | C |
| ATOM | 1643 | CD1 | TYR | B | 299 | −7.783 | 20.008 | 25.370 | 1.00 | 50.00 | C |
| ATOM | 1644 | CD2 | TYR | B | 299 | −6.995 | 19.970 | 23.188 | 1.00 | 50.00 | C |
| ATOM | 1645 | CE1 | TYR | B | 299 | −8.967 | 20.490 | 24.948 | 1.00 | 50.00 | C |
| ATOM | 1646 | CE2 | TYR | B | 299 | −8.184 | 20.430 | 22.747 | 1.00 | 50.00 | C |
| ATOM | 1647 | CZ | TYR | B | 299 | −9.175 | 20.668 | 23.629 | 1.00 | 50.00 | C |
| ATOM | 1648 | OH | TYR | B | 299 | −10.405 | 21.083 | 23.191 | 1.00 | 50.00 | O |
| ATOM | 1649 | N | VAL | B | 300 | −5.451 | 21.641 | 27.043 | 1.00 | 50.00 | N |
| ATOM | 1650 | CA | VAL | B | 300 | −6.345 | 22.656 | 27.603 | 1.00 | 50.00 | C |
| ATOM | 1651 | C | VAL | B | 300 | −6.751 | 23.569 | 26.452 | 1.00 | 50.00 | C |
| ATOM | 1652 | O | VAL | B | 300 | −5.986 | 24.459 | 26.082 | 1.00 | 50.00 | O |
| ATOM | 1653 | CB | VAL | B | 300 | −5.728 | 23.446 | 28.762 | 1.00 | 50.00 | C |
| ATOM | 1654 | CG1 | VAL | B | 300 | −6.465 | 24.706 | 29.215 | 1.00 | 50.00 | C |
| ATOM | 1655 | CG2 | VAL | B | 300 | −5.575 | 22.630 | 30.043 | 1.00 | 50.00 | C |
| ATOM | 1656 | N | VAL | B | 301 | −7.989 | 23.461 | 25.998 | 1.00 | 50.00 | N |
| ATOM | 1657 | CA | VAL | B | 301 | −8.675 | 24.491 | 25.214 | 1.00 | 50.00 | C |
| ATOM | 1658 | C | VAL | B | 301 | −9.045 | 25.715 | 26.043 | 1.00 | 50.00 | C |
| ATOM | 1659 | O | VAL | B | 301 | −9.153 | 25.701 | 27.269 | 1.00 | 50.00 | O |
| ATOM | 1660 | CB | VAL | B | 301 | −9.880 | 23.916 | 24.468 | 1.00 | 50.00 | C |
| ATOM | 1661 | CG1 | VAL | B | 301 | −10.953 | 23.425 | 25.436 | 1.00 | 50.00 | C |
| ATOM | 1662 | CG2 | VAL | B | 301 | −10.525 | 24.684 | 23.314 | 1.00 | 50.00 | C |
| ATOM | 1663 | N | GLN | B | 302 | −9.278 | 26.777 | 25.292 | 1.00 | 50.00 | N |
| ATOM | 1664 | CA | GLN | B | 302 | −9.634 | 28.098 | 25.804 | 1.00 | 50.00 | C |
| ATOM | 1665 | C | GLN | B | 302 | −10.625 | 28.656 | 24.792 | 1.00 | 50.00 | C |
| ATOM | 1666 | O | GLN | B | 302 | −10.220 | 29.169 | 23.749 | 1.00 | 50.00 | O |
| ATOM | 1667 | CB | GLN | B | 302 | −8.356 | 28.930 | 25.926 | 1.00 | 50.00 | C |
| ATOM | 1668 | CG | GLN | B | 302 | −7.428 | 28.538 | 27.076 | 1.00 | 50.00 | C |
| ATOM | 1669 | CD | GLN | B | 302 | −8.005 | 28.724 | 28.473 | 1.00 | 50.00 | C |
| ATOM | 1670 | OE1 | GLN | B | 302 | −9.021 | 29.381 | 28.696 | 1.00 | 50.00 | O |
| ATOM | 1671 | NE2 | GLN | B | 302 | −7.368 | 28.139 | 29.471 | 1.00 | 50.00 | N |
| ATOM | 1672 | N | LEU | B | 303 | −11.910 | 28.531 | 25.083 | 1.00 | 50.00 | N |
| ATOM | 1673 | CA | LEU | B | 303 | −12.962 | 29.036 | 24.202 | 1.00 | 50.00 | C |
| ATOM | 1674 | C | LEU | B | 303 | −13.343 | 30.475 | 24.532 | 1.00 | 50.00 | C |
| ATOM | 1675 | O | LEU | B | 303 | −13.365 | 30.849 | 25.705 | 1.00 | 50.00 | O |
| ATOM | 1676 | CB | LEU | B | 303 | −14.222 | 28.191 | 24.014 | 1.00 | 50.00 | C |
| ATOM | 1677 | CG | LEU | B | 303 | −14.017 | 26.812 | 23.389 | 1.00 | 50.00 | C |
| ATOM | 1678 | CD1 | LEU | B | 303 | −13.558 | 25.750 | 24.384 | 1.00 | 50.00 | C |
| ATOM | 1679 | CD2 | LEU | B | 303 | −15.316 | 26.339 | 22.742 | 1.00 | 50.00 | C |
| ATOM | 1680 | N | PRO | B | 304 | −13.684 | 31.301 | 23.555 | 1.00 | 50.00 | N |
| ATOM | 1681 | CA | PRO | B | 304 | −14.159 | 32.668 | 23.767 | 1.00 | 50.00 | C |
| ATOM | 1682 | C | PRO | B | 304 | −15.673 | 32.801 | 23.872 | 1.00 | 50.00 | C |
| ATOM | 1683 | O | PRO | B | 304 | −16.401 | 31.820 | 23.724 | 1.00 | 50.00 | O |
| ATOM | 1684 | CB | PRO | B | 304 | −13.620 | 33.393 | 22.537 | 1.00 | 50.00 | C |
| ATOM | 1685 | CG | PRO | B | 304 | −13.808 | 32.299 | 21.493 | 1.00 | 50.00 | C |
| ATOM | 1686 | CD | PRO | B | 304 | −13.604 | 30.938 | 22.142 | 1.00 | 50.00 | C |
| ATOM | 1687 | N | LEU | B | 305 | −16.171 | 33.978 | 24.208 | 1.00 | 50.00 | N |
| ATOM | 1688 | CA | LEU | B | 305 | −17.592 | 34.153 | 24.506 | 1.00 | 50.00 | C |
| ATOM | 1689 | C | LEU | B | 305 | −18.043 | 35.423 | 23.808 | 1.00 | 50.00 | C |
| ATOM | 1690 | O | LEU | B | 305 | −17.949 | 36.549 | 24.296 | 1.00 | 50.00 | O |
| ATOM | 1691 | CB | LEU | B | 305 | −17.932 | 34.296 | 25.986 | 1.00 | 50.00 | C |
| ATOM | 1692 | CG | LEU | B | 305 | −17.880 | 32.994 | 26.779 | 1.00 | 50.00 | C |
| ATOM | 1693 | CD1 | LEU | B | 305 | −16.430 | 32.560 | 26.977 | 1.00 | 50.00 | C |
| ATOM | 1694 | CD2 | LEU | B | 305 | −18.637 | 33.279 | 28.073 | 1.00 | 50.00 | C |
| ATOM | 1695 | N | TYR | B | 306 | −18.592 | 35.133 | 22.643 | 1.00 | 50.00 | N |
| ATOM | 1696 | CA | TYR | B | 306 | −19.035 | 36.183 | 21.735 | 1.00 | 50.00 | C |
| ATOM | 1697 | C | TYR | B | 306 | −20.452 | 36.624 | 22.074 | 1.00 | 50.00 | C |
| ATOM | 1698 | O | TYR | B | 306 | −21.431 | 35.867 | 21.968 | 1.00 | 50.00 | O |
| ATOM | 1699 | CB | TYR | B | 306 | −18.707 | 35.643 | 20.347 | 1.00 | 50.00 | C |
| ATOM | 1700 | CG | TYR | B | 306 | −19.221 | 36.498 | 19.215 | 1.00 | 50.00 | C |
| ATOM | 1701 | CD1 | TYR | B | 306 | −18.606 | 37.682 | 19.030 | 1.00 | 50.00 | C |
| ATOM | 1702 | CD2 | TYR | B | 306 | −20.031 | 36.010 | 18.255 | 1.00 | 50.00 | C |
| ATOM | 1703 | CE1 | TYR | B | 306 | −18.492 | 38.153 | 17.774 | 1.00 | 50.00 | C |
| ATOM | 1704 | CE2 | TYR | B | 306 | −20.158 | 36.703 | 17.105 | 1.00 | 50.00 | C |
| ATOM | 1705 | CZ | TYR | B | 306 | −19.305 | 37.699 | 16.803 | 1.00 | 50.00 | C |
| ATOM | 1706 | OH | TYR | B | 306 | −19.466 | 38.494 | 15.701 | 1.00 | 50.00 | O |
| ATOM | 1707 | N | GLY | B | 307 | −20.475 | 37.877 | 22.493 | 1.00 | 50.00 | N |
| ATOM | 1708 | CA | GLY | B | 307 | −21.692 | 38.665 | 22.665 | 1.00 | 50.00 | C |
| ATOM | 1709 | C | GLY | B | 307 | −21.661 | 39.751 | 21.601 | 1.00 | 50.00 | C |
| ATOM | 1710 | O | GLY | B | 307 | −20.939 | 40.740 | 21.718 | 1.00 | 50.00 | O |
| ATOM | 1711 | N | VAL | B | 308 | −22.484 | 39.557 | 20.585 | 1.00 | 50.00 | N |
| ATOM | 1712 | CA | VAL | B | 308 | −22.759 | 40.551 | 19.547 | 1.00 | 50.00 | C |
| ATOM | 1713 | C | VAL | B | 308 | −23.193 | 41.921 | 20.041 | 1.00 | 50.00 | C |
| ATOM | 1714 | O | VAL | B | 308 | −23.869 | 42.084 | 21.057 | 1.00 | 50.00 | O |
| ATOM | 1715 | CB | VAL | B | 308 | −23.764 | 40.042 | 18.514 | 1.00 | 50.00 | C |
| ATOM | 1716 | CG1 | VAL | B | 308 | −25.143 | 39.743 | 19.102 | 1.00 | 50.00 | C |
| ATOM | 1717 | CG2 | VAL | B | 308 | −23.839 | 40.584 | 17.089 | 1.00 | 50.00 | C |
| ATOM | 1718 | N | ILE | B | 309 | −22.924 | 42.849 | 19.141 | 1.00 | 50.00 | N |
| ATOM | 1719 | CA | ILE | B | 309 | −23.699 | 44.084 | 19.035 | 1.00 | 50.00 | C |
| ATOM | 1720 | C | ILE | B | 309 | −24.866 | 43.784 | 18.100 | 1.00 | 50.00 | C |
| ATOM | 1721 | O | ILE | B | 309 | −24.761 | 43.957 | 16.886 | 1.00 | 50.00 | O |
| ATOM | 1722 | CB | ILE | B | 309 | −22.774 | 45.228 | 18.615 | 1.00 | 50.00 | C |
| ATOM | 1723 | CG1 | ILE | B | 309 | −21.567 | 45.375 | 19.541 | 1.00 | 50.00 | C |
| ATOM | 1724 | CG2 | ILE | B | 309 | −23.564 | 46.536 | 18.595 | 1.00 | 50.00 | C |
| ATOM | 1725 | CD1 | ILE | B | 309 | −20.491 | 46.353 | 19.081 | 1.00 | 50.00 | C |
| ATOM | 1726 | N | ASP | B | 310 | −25.954 | 43.317 | 18.690 | 1.00 | 50.00 | N |
| ATOM | 1727 | CA | ASP | B | 310 | −27.241 | 43.070 | 18.037 | 1.00 | 50.00 | C |
| ATOM | 1728 | C | ASP | B | 310 | −28.005 | 44.351 | 17.726 | 1.00 | 50.00 | C |
| ATOM | 1729 | O | ASP | B | 310 | −29.025 | 44.727 | 18.303 | 1.00 | 50.00 | O |
| ATOM | 1730 | CB | ASP | B | 310 | −28.080 | 42.078 | 18.844 | 1.00 | 50.00 | C |
| ATOM | 1731 | CG | ASP | B | 310 | −28.318 | 42.396 | 20.314 | 1.00 | 50.00 | C |
| ATOM | 1732 | OD1 | ASP | B | 310 | −27.445 | 42.072 | 21.147 | 1.00 | 50.00 | O |
| ATOM | 1733 | OD2 | ASP | B | 310 | −29.361 | 43.001 | 20.645 | 1.00 | 50.00 | O |
| ATOM | 1734 | N | THR | B | 311 | −27.444 | 45.024 | 16.739 | 1.00 | 50.00 | N |
| ATOM | 1735 | CA | THR | B | 311 | −27.961 | 46.216 | 16.071 | 1.00 | 50.00 | C |
| ATOM | 1736 | C | THR | B | 311 | −27.789 | 45.788 | 14.616 | 1.00 | 50.00 | C |
| ATOM | 1737 | O | THR | B | 311 | −26.677 | 45.364 | 14.295 | 1.00 | 50.00 | O |
| ATOM | 1738 | CB | THR | B | 311 | −27.185 | 47.470 | 16.486 | 1.00 | 50.00 | C |
| ATOM | 1739 | OG1 | THR | B | 311 | −25.853 | 47.480 | 15.989 | 1.00 | 50.00 | O |
| ATOM | 1740 | CG2 | THR | B | 311 | −27.084 | 47.742 | 17.986 | 1.00 | 50.00 | C |
| ATOM | 1741 | N | PRO | B | 312 | −28.719 | 45.859 | 13.673 | 1.00 | 50.00 | N |
| ATOM | 1742 | CA | PRO | B | 312 | −28.409 | 45.642 | 12.258 | 1.00 | 50.00 | C |
| ATOM | 1743 | C | PRO | B | 312 | −27.642 | 46.792 | 11.616 | 1.00 | 50.00 | C |
| ATOM | 1744 | O | PRO | B | 312 | −28.069 | 47.449 | 10.667 | 1.00 | 50.00 | O |
| ATOM | 1745 | CB | PRO | B | 312 | −29.766 | 45.281 | 11.660 | 1.00 | 50.00 | C |
| ATOM | 1746 | CG | PRO | B | 312 | −30.794 | 45.991 | 12.536 | 1.00 | 50.00 | C |
| ATOM | 1747 | CD | PRO | B | 312 | −30.121 | 46.209 | 13.890 | 1.00 | 50.00 | C |
| ATOM | 1748 | N | CYS | B | 313 | −26.504 | 47.074 | 12.228 | 1.00 | 50.00 | N |
| ATOM | 1749 | CA | CYS | B | 313 | −25.220 | 47.292 | 11.569 | 1.00 | 50.00 | C |
| ATOM | 1750 | C | CYS | B | 313 | −23.955 | 47.058 | 12.387 | 1.00 | 50.00 | C |
| ATOM | 1751 | O | CYS | B | 313 | −24.040 | 46.918 | 13.606 | 1.00 | 50.00 | O |
| ATOM | 1752 | CB | CYS | B | 313 | −25.258 | 48.726 | 11.059 | 1.00 | 50.00 | C |
| ATOM | 1753 | SG | CYS | B | 313 | −26.464 | 49.726 | 11.951 | 1.00 | 50.00 | S |
| ATOM | 1754 | N | TRP | B | 314 | −22.729 | 47.255 | 11.927 | 1.00 | 50.00 | N |
| ATOM | 1755 | CA | TRP | B | 314 | −22.111 | 46.860 | 10.659 | 1.00 | 50.00 | C |
| ATOM | 1756 | C | TRP | B | 314 | −20.908 | 47.783 | 10.467 | 1.00 | 50.00 | C |
| ATOM | 1757 | O | TRP | B | 314 | −21.111 | 48.989 | 10.333 | 1.00 | 50.00 | O |
| ATOM | 1758 | CB | TRP | B | 314 | −22.893 | 46.583 | 9.367 | 1.00 | 50.00 | C |
| ATOM | 1759 | CG | TRP | B | 314 | −23.898 | 45.459 | 9.193 | 1.00 | 50.00 | C |
| ATOM | 1760 | CD1 | TRP | B | 314 | −23.976 | 44.449 | 10.146 | 1.00 | 50.00 | C |
| ATOM | 1761 | CD2 | TRP | B | 314 | −25.135 | 45.374 | 8.552 | 1.00 | 50.00 | C |
| ATOM | 1762 | NE1 | TRP | B | 314 | −25.108 | 43.665 | 10.064 | 1.00 | 50.00 | N |
| ATOM | 1763 | CE2 | TRP | B | 314 | −25.833 | 44.275 | 9.068 | 1.00 | 50.00 | C |
| ATOM | 1764 | CE3 | TRP | B | 314 | −25.845 | 46.327 | 7.902 | 1.00 | 50.00 | C |
| ATOM | 1765 | CZ2 | TRP | B | 314 | −27.175 | 44.170 | 8.951 | 1.00 | 50.00 | C |
| ATOM | 1766 | CZ3 | TRP | B | 314 | −27.183 | 46.204 | 7.781 | 1.00 | 50.00 | C |
| ATOM | 1767 | CH2 | TRP | B | 314 | −27.838 | 45.145 | 8.300 | 1.00 | 50.00 | C |
| ATOM | 1768 | N | LYS | B | 315 | −19.675 | 47.295 | 10.433 | 1.00 | 50.00 | N |
| ATOM | 1769 | CA | LYS | B | 315 | −18.457 | 47.957 | 9.955 | 1.00 | 50.00 | C |
| ATOM | 1770 | C | LYS | B | 315 | −18.259 | 47.872 | 8.446 | 1.00 | 50.00 | C |
| ATOM | 1771 | O | LYS | B | 315 | −18.376 | 46.788 | 7.879 | 1.00 | 50.00 | O |
| ATOM | 1772 | CB | LYS | B | 315 | −17.180 | 47.340 | 10.529 | 1.00 | 50.00 | C |
| ATOM | 1773 | CG | LYS | B | 315 | −16.979 | 47.579 | 12.022 | 1.00 | 50.00 | C |
| ATOM | 1774 | CD | LYS | B | 315 | −15.722 | 46.980 | 12.647 | 1.00 | 50.00 | C |
| ATOM | 1775 | CE | LYS | B | 315 | −15.722 | 47.262 | 14.147 | 1.00 | 50.00 | C |
| ATOM | 1776 | NZ | LYS | B | 315 | −14.474 | 46.857 | 14.807 | 1.00 | 50.00 | N |
| ATOM | 1777 | N | LEU | B | 316 | −17.964 | 48.976 | 7.780 | 1.00 | 50.00 | N |
| ATOM | 1778 | CA | LEU | B | 316 | −18.037 | 49.084 | 6.321 | 1.00 | 50.00 | C |
| ATOM | 1779 | C | LEU | B | 316 | −16.727 | 49.672 | 5.807 | 1.00 | 50.00 | C |
| ATOM | 1780 | O | LEU | B | 316 | −16.481 | 50.869 | 5.943 | 1.00 | 50.00 | O |
| ATOM | 1781 | CB | LEU | B | 316 | −19.286 | 49.851 | 5.879 | 1.00 | 50.00 | C |
| ATOM | 1782 | CG | LEU | B | 316 | −20.649 | 49.158 | 5.957 | 1.00 | 50.00 | C |
| ATOM | 1783 | CD1 | LEU | B | 316 | −21.238 | 49.028 | 7.359 | 1.00 | 50.00 | C |
| ATOM | 1784 | CD2 | LEU | B | 316 | −21.748 | 49.844 | 5.148 | 1.00 | 50.00 | C |
| ATOM | 1785 | N | HIS | B | 317 | −15.893 | 48.849 | 5.190 | 1.00 | 50.00 | N |
| ATOM | 1786 | CA | HIS | B | 317 | −14.608 | 49.224 | 4.598 | 1.00 | 50.00 | C |
| ATOM | 1787 | C | HIS | B | 317 | −14.780 | 50.035 | 3.318 | 1.00 | 50.00 | C |
| ATOM | 1788 | O | HIS | B | 317 | −15.501 | 49.645 | 2.400 | 1.00 | 50.00 | O |
| ATOM | 1789 | CB | HIS | B | 317 | −13.748 | 47.982 | 4.354 | 1.00 | 50.00 | C |
| ATOM | 1790 | CG | HIS | B | 317 | −12.276 | 48.210 | 4.085 | 1.00 | 50.00 | C |
| ATOM | 1791 | ND1 | HIS | B | 317 | −11.290 | 48.042 | 5.039 | 1.00 | 50.00 | N |
| ATOM | 1792 | CD2 | HIS | B | 317 | −11.714 | 48.615 | 2.877 | 1.00 | 50.00 | C |
| ATOM | 1793 | CE1 | HIS | B | 317 | −10.184 | 48.386 | 4.291 | 1.00 | 50.00 | C |
| ATOM | 1794 | NE2 | HIS | B | 317 | −10.339 | 48.700 | 2.958 | 1.00 | 50.00 | N |
| ATOM | 1795 | N | THR | B | 318 | −14.064 | 51.146 | 3.271 | 1.00 | 50.00 | N |
| ATOM | 1796 | CA | THR | B | 318 | −13.993 | 52.067 | 2.136 | 1.00 | 50.00 | C |
| ATOM | 1797 | C | THR | B | 318 | −12.571 | 52.067 | 1.585 | 1.00 | 50.00 | C |
| ATOM | 1798 | O | THR | B | 318 | −11.592 | 52.169 | 2.323 | 1.00 | 50.00 | O |
| ATOM | 1799 | CB | THR | B | 318 | −14.447 | 53.443 | 2.627 | 1.00 | 50.00 | C |
| ATOM | 1800 | OG1 | THR | B | 318 | −15.832 | 53.417 | 2.940 | 1.00 | 50.00 | O |
| ATOM | 1801 | CG2 | THR | B | 318 | −14.235 | 54.620 | 1.676 | 1.00 | 50.00 | C |
| ATOM | 1802 | N | SER | B | 319 | −12.474 | 52.049 | 0.265 | 1.00 | 50.00 | N |
| ATOM | 1803 | CA | SER | B | 319 | −11.264 | 52.386 | −0.486 | 1.00 | 50.00 | C |
| ATOM | 1804 | C | SER | B | 319 | −11.569 | 53.424 | −1.562 | 1.00 | 50.00 | C |
| ATOM | 1805 | O | SER | B | 319 | −12.517 | 53.185 | −2.309 | 1.00 | 50.00 | O |
| ATOM | 1806 | CB | SER | B | 319 | −10.666 | 51.153 | −1.161 | 1.00 | 50.00 | C |
| ATOM | 1807 | OG | SER | B | 319 | −10.190 | 50.230 | −0.192 | 1.00 | 50.00 | O |
| ATOM | 1808 | N | PRO | B | 320 | −10.829 | 54.505 | −1.768 | 1.00 | 50.00 | N |
| ATOM | 1809 | CA | PRO | B | 320 | −11.053 | 55.437 | −2.873 | 1.00 | 50.00 | C |
| ATOM | 1810 | C | PRO | B | 320 | −10.114 | 55.276 | −4.063 | 1.00 | 50.00 | C |
| ATOM | 1811 | O | PRO | B | 320 | −8.892 | 55.320 | −3.923 | 1.00 | 50.00 | O |
| ATOM | 1812 | CB | PRO | B | 320 | −10.939 | 56.796 | −2.186 | 1.00 | 50.00 | C |
| ATOM | 1813 | CG | PRO | B | 320 | −9.904 | 56.585 | −1.084 | 1.00 | 50.00 | C |
| ATOM | 1814 | CD | PRO | B | 320 | −9.944 | 55.093 | −0.763 | 1.00 | 50.00 | C |
| ATOM | 1815 | N | LEU | B | 321 | −10.708 | 55.177 | −5.241 | 1.00 | 50.00 | N |
| ATOM | 1816 | CA | LEU | B | 321 | −10.008 | 55.343 | −6.514 | 1.00 | 50.00 | C |
| ATOM | 1817 | C | LEU | B | 321 | −9.840 | 56.814 | −6.870 | 1.00 | 50.00 | C |
| ATOM | 1818 | O | LEU | B | 321 | −10.571 | 57.723 | −6.476 | 1.00 | 50.00 | O |
| ATOM | 1819 | CB | LEU | B | 321 | −10.732 | 54.644 | −7.667 | 1.00 | 50.00 | C |
| ATOM | 1820 | CG | LEU | B | 321 | −10.494 | 53.148 | −7.870 | 1.00 | 50.00 | C |
| ATOM | 1821 | CD1 | LEU | B | 321 | −9.040 | 52.855 | −8.231 | 1.00 | 50.00 | C |
| ATOM | 1822 | CD2 | LEU | B | 321 | −10.971 | 52.299 | −6.694 | 1.00 | 50.00 | C |
| ATOM | 1823 | N | CYS | B | 322 | −8.862 | 56.966 | −7.744 | 1.00 | 50.00 | N |
| ATOM | 1824 | CA | CYS | B | 322 | −8.587 | 58.200 | −8.474 | 1.00 | 50.00 | C |
| ATOM | 1825 | C | CYS | B | 322 | −9.389 | 58.353 | −9.765 | 1.00 | 50.00 | C |
| ATOM | 1826 | O | CYS | B | 322 | −8.928 | 57.973 | −10.840 | 1.00 | 50.00 | O |
| ATOM | 1827 | CB | CYS | B | 322 | −7.061 | 58.117 | −8.497 | 1.00 | 50.00 | C |
| ATOM | 1828 | SG | CYS | B | 322 | −6.438 | 58.685 | −6.902 | 1.00 | 50.00 | S |
| ATOM | 1829 | N | THR | B | 323 | −10.530 | 59.022 | −9.676 | 1.00 | 50.00 | N |
| ATOM | 1830 | CA | THR | B | 323 | −11.310 | 59.605 | −10.773 | 1.00 | 50.00 | C |
| ATOM | 1831 | C | THR | B | 323 | −10.988 | 61.026 | −11.226 | 1.00 | 50.00 | C |
| ATOM | 1832 | O | THR | B | 323 | −10.769 | 61.909 | −10.400 | 1.00 | 50.00 | O |
| ATOM | 1833 | CB | THR | B | 323 | −12.758 | 59.654 | −10.280 | 1.00 | 50.00 | C |
| ATOM | 1834 | OG1 | THR | B | 323 | −13.247 | 58.322 | −10.216 | 1.00 | 50.00 | O |
| ATOM | 1835 | CG2 | THR | B | 323 | −13.805 | 60.491 | −11.020 | 1.00 | 50.00 | C |
| ATOM | 1836 | N | THR | B | 324 | −11.285 | 61.451 | −12.443 | 1.00 | 50.00 | N |
| ATOM | 1837 | CA | THR | B | 324 | −11.338 | 62.873 | −12.796 | 1.00 | 50.00 | C |
| ATOM | 1838 | C | THR | B | 324 | −12.424 | 63.696 | −12.106 | 1.00 | 50.00 | C |
| ATOM | 1839 | O | THR | B | 324 | −12.191 | 64.108 | −10.970 | 1.00 | 50.00 | O |
| ATOM | 1840 | CB | THR | B | 324 | −11.084 | 63.011 | −14.297 | 1.00 | 50.00 | C |
| ATOM | 1841 | OG1 | THR | B | 324 | −9.871 | 62.365 | −14.660 | 1.00 | 50.00 | O |
| ATOM | 1842 | CG2 | THR | B | 324 | −10.938 | 64.451 | −14.785 | 1.00 | 50.00 | C |
| ATOM | 1843 | N | ASN | B | 325 | −13.569 | 63.987 | −12.704 | 1.00 | 50.00 | N |
| ATOM | 1844 | CA | ASN | B | 325 | −14.592 | 64.894 | −12.175 | 1.00 | 50.00 | C |
| ATOM | 1845 | C | ASN | B | 325 | −14.082 | 66.298 | −11.857 | 1.00 | 50.00 | C |
| ATOM | 1846 | O | ASN | B | 325 | −13.916 | 67.102 | −12.773 | 1.00 | 50.00 | O |
| ATOM | 1847 | CB | ASN | B | 325 | −15.505 | 64.248 | −11.130 | 1.00 | 50.00 | C |
| ATOM | 1848 | CG | ASN | B | 325 | −16.402 | 63.065 | −11.472 | 1.00 | 50.00 | C |
| ATOM | 1849 | OD1 | ASN | B | 325 | −16.663 | 62.217 | −10.620 | 1.00 | 50.00 | O |
| ATOM | 1850 | ND2 | ASN | B | 325 | −16.955 | 62.974 | −12.668 | 1.00 | 50.00 | N |
| ATOM | 1851 | N | THR | B | 326 | −13.783 | 66.584 | −10.600 | 1.00 | 50.00 | N |
| ATOM | 1852 | CA | THR | B | 326 | −12.936 | 67.695 | −10.161 | 1.00 | 50.00 | C |
| ATOM | 1853 | C | THR | B | 326 | −11.583 | 67.101 | −9.782 | 1.00 | 50.00 | C |
| ATOM | 1854 | O | THR | B | 326 | −11.501 | 66.231 | −8.915 | 1.00 | 50.00 | O |
| ATOM | 1855 | CB | THR | B | 326 | −13.632 | 68.423 | −9.008 | 1.00 | 50.00 | C |
| ATOM | 1856 | OG1 | THR | B | 326 | −14.845 | 69.011 | −9.458 | 1.00 | 50.00 | O |
| ATOM | 1857 | CG2 | THR | B | 326 | −12.825 | 69.538 | −8.347 | 1.00 | 50.00 | C |
| ATOM | 1858 | N | LYS | B | 327 | −10.522 | 67.556 | −10.429 | 1.00 | 50.00 | N |
| ATOM | 1859 | CA | LYS | B | 327 | −9.172 | 67.001 | −10.308 | 1.00 | 50.00 | C |
| ATOM | 1860 | C | LYS | B | 327 | −8.121 | 68.105 | −10.383 | 1.00 | 50.00 | C |
| ATOM | 1861 | O | LYS | B | 327 | −8.360 | 69.167 | −10.958 | 1.00 | 50.00 | O |
| ATOM | 1862 | CB | LYS | B | 327 | −9.008 | 65.970 | −11.427 | 1.00 | 50.00 | C |
| ATOM | 1863 | CG | LYS | B | 327 | −7.865 | 64.968 | −11.273 | 1.00 | 50.00 | C |
| ATOM | 1864 | CD | LYS | B | 327 | −7.601 | 64.043 | −12.460 | 1.00 | 50.00 | C |
| ATOM | 1865 | CE | LYS | B | 327 | −6.535 | 63.014 | −12.097 | 1.00 | 50.00 | C |
| ATOM | 1866 | NZ | LYS | B | 327 | −6.011 | 62.124 | −13.141 | 1.00 | 50.00 | N |
| ATOM | 1867 | N | GLU | B | 328 | −6.931 | 67.867 | −9.854 | 1.00 | 50.00 | N |
| ATOM | 1868 | CA | GLU | B | 328 | −5.747 | 68.696 | −10.092 | 1.00 | 50.00 | C |
| ATOM | 1869 | C | GLU | B | 328 | −4.601 | 67.763 | −10.468 | 1.00 | 50.00 | C |
| ATOM | 1870 | O | GLU | B | 328 | −4.107 | 67.018 | −9.623 | 1.00 | 50.00 | O |
| ATOM | 1871 | CB | GLU | B | 328 | −5.408 | 69.616 | −8.916 | 1.00 | 50.00 | C |
| ATOM | 1872 | CG | GLU | B | 328 | −6.482 | 70.575 | −8.402 | 1.00 | 50.00 | C |
| ATOM | 1873 | CD | GLU | B | 328 | −6.579 | 71.852 | −9.222 | 1.00 | 50.00 | C |
| ATOM | 1874 | OE1 | GLU | B | 328 | −7.263 | 71.852 | −10.269 | 1.00 | 50.00 | O |
| ATOM | 1875 | OE2 | GLU | B | 328 | −5.958 | 72.864 | −8.831 | 1.00 | 50.00 | O |
| ATOM | 1876 | N | GLY | B | 329 | −4.153 | 67.724 | −11.712 | 1.00 | 50.00 | N |
| ATOM | 1877 | CA | GLY | B | 329 | −3.078 | 66.833 | −12.153 | 1.00 | 50.00 | C |
| ATOM | 1878 | C | GLY | B | 329 | −3.409 | 65.346 | −12.194 | 1.00 | 50.00 | C |
| ATOM | 1879 | O | GLY | B | 329 | −4.388 | 64.986 | −12.845 | 1.00 | 50.00 | O |
| ATOM | 1880 | N | SER | B | 330 | −2.630 | 64.514 | −11.521 | 1.00 | 50.00 | N |
| ATOM | 1881 | CA | SER | B | 330 | −3.010 | 63.148 | −11.151 | 1.00 | 50.00 | C |
| ATOM | 1882 | C | SER | B | 330 | −3.685 | 62.965 | −9.794 | 1.00 | 50.00 | C |
| ATOM | 1883 | O | SER | B | 330 | −3.796 | 61.834 | −9.321 | 1.00 | 50.00 | O |
| ATOM | 1884 | CB | SER | B | 330 | −1.797 | 62.223 | −11.246 | 1.00 | 50.00 | C |
| ATOM | 1885 | OG | SER | B | 330 | −1.339 | 62.109 | −12.586 | 1.00 | 50.00 | O |
| ATOM | 1886 | N | ASN | B | 331 | −4.188 | 64.017 | −9.168 | 1.00 | 50.00 | N |
| ATOM | 1887 | CA | ASN | B | 331 | −4.797 | 63.972 | −7.840 | 1.00 | 50.00 | C |
| ATOM | 1888 | C | ASN | B | 331 | −6.303 | 64.162 | −7.947 | 1.00 | 50.00 | C |
| ATOM | 1889 | O | ASN | B | 331 | −6.803 | 65.127 | −8.524 | 1.00 | 50.00 | O |
| ATOM | 1890 | CB | ASN | B | 331 | −4.139 | 64.960 | −6.878 | 1.00 | 50.00 | C |
| ATOM | 1891 | CG | ASN | B | 331 | −4.300 | 66.473 | −6.888 | 1.00 | 50.00 | C |
| ATOM | 1892 | OD1 | ASN | B | 331 | −3.317 | 67.211 | −6.927 | 1.00 | 50.00 | O |
| ATOM | 1893 | ND2 | ASN | B | 331 | −5.514 | 66.987 | −6.812 | 1.00 | 50.00 | N |
| ATOM | 1894 | N | ILE | B | 332 | −7.023 | 63.239 | −7.335 | 1.00 | 50.00 | N |
| ATOM | 1895 | CA | ILE | B | 332 | −8.482 | 63.181 | −7.414 | 1.00 | 50.00 | C |
| ATOM | 1896 | C | ILE | B | 332 | −8.732 | 63.706 | −6.011 | 1.00 | 50.00 | C |
| ATOM | 1897 | O | ILE | B | 332 | −8.566 | 64.917 | −5.869 | 1.00 | 50.00 | O |
| ATOM | 1898 | CB | ILE | B | 332 | −9.068 | 61.859 | −7.910 | 1.00 | 50.00 | C |
| ATOM | 1899 | CG1 | ILE | B | 332 | −8.380 | 61.536 | −9.239 | 1.00 | 50.00 | C |
| ATOM | 1900 | CG2 | ILE | B | 332 | −10.589 | 61.838 | −7.727 | 1.00 | 50.00 | C |
| ATOM | 1901 | CD1 | ILE | B | 332 | −6.909 | 61.135 | −9.343 | 1.00 | 50.00 | C |
| ATOM | 1902 | N | CYS | B | 333 | −9.057 | 63.041 | −4.916 | 1.00 | 50.00 | N |
| ATOM | 1903 | CA | CYS | B | 333 | −9.218 | 61.605 | −4.684 | 1.00 | 50.00 | C |
| ATOM | 1904 | C | CYS | B | 333 | −9.701 | 61.686 | −3.240 | 1.00 | 50.00 | C |
| ATOM | 1905 | O | CYS | B | 333 | −9.074 | 62.264 | −2.352 | 1.00 | 50.00 | O |
| ATOM | 1906 | CB | CYS | B | 333 | −8.024 | 60.654 | −4.748 | 1.00 | 50.00 | C |
| ATOM | 1907 | SG | CYS | B | 333 | −6.952 | 60.491 | −6.191 | 1.00 | 50.00 | S |
| ATOM | 1908 | N | LEU | B | 334 | −10.866 | 61.111 | −2.999 | 1.00 | 50.00 | N |
| ATOM | 1909 | CA | LEU | B | 334 | −11.755 | 61.572 | −1.934 | 1.00 | 50.00 | C |
| ATOM | 1910 | C | LEU | B | 334 | −11.265 | 61.320 | −0.515 | 1.00 | 50.00 | C |
| ATOM | 1911 | O | LEU | B | 334 | −10.562 | 60.348 | −0.240 | 1.00 | 50.00 | O |
| ATOM | 1912 | CB | LEU | B | 334 | −13.125 | 60.942 | −2.178 | 1.00 | 50.00 | C |
| ATOM | 1913 | CG | LEU | B | 334 | −13.904 | 61.688 | −3.259 | 1.00 | 50.00 | C |
| ATOM | 1914 | CD1 | LEU | B | 334 | −13.424 | 61.487 | −4.696 | 1.00 | 50.00 | C |
| ATOM | 1915 | CD2 | LEU | B | 334 | −15.362 | 61.260 | −3.168 | 1.00 | 50.00 | C |
| ATOM | 1916 | N | THR | B | 335 | −11.651 | 62.237 | 0.358 | 1.00 | 50.00 | N |
| ATOM | 1917 | CA | THR | B | 335 | −11.246 | 62.219 | 1.763 | 1.00 | 50.00 | C |
| ATOM | 1918 | C | THR | B | 335 | −12.381 | 61.570 | 2.543 | 1.00 | 50.00 | C |
| ATOM | 1919 | O | THR | B | 335 | −13.383 | 62.199 | 2.882 | 1.00 | 50.00 | O |
| ATOM | 1920 | CB | THR | B | 335 | −10.885 | 63.606 | 2.292 | 1.00 | 50.00 | C |
| ATOM | 1921 | OG1 | THR | B | 335 | −9.780 | 64.112 | 1.557 | 1.00 | 50.00 | O |
| ATOM | 1922 | CG2 | THR | B | 335 | −10.504 | 63.601 | 3.771 | 1.00 | 50.00 | C |
| ATOM | 1923 | N | ARG | B | 336 | −12.154 | 60.299 | 2.826 | 1.00 | 50.00 | N |
| ATOM | 1924 | CA | ARG | B | 336 | −12.990 | 59.499 | 3.717 | 1.00 | 50.00 | C |
| ATOM | 1925 | C | ARG | B | 336 | −12.070 | 58.510 | 4.419 | 1.00 | 50.00 | C |
| ATOM | 1926 | O | ARG | B | 336 | −10.905 | 58.283 | 4.093 | 1.00 | 50.00 | O |
| ATOM | 1927 | CB | ARG | B | 336 | −14.131 | 58.789 | 2.988 | 1.00 | 50.00 | C |
| ATOM | 1928 | CG | ARG | B | 336 | −15.044 | 59.766 | 2.253 | 1.00 | 50.00 | C |
| ATOM | 1929 | CD | ARG | B | 336 | −16.297 | 59.101 | 1.698 | 1.00 | 50.00 | C |
| ATOM | 1930 | NE | ARG | B | 336 | −17.039 | 60.147 | 0.996 | 1.00 | 50.00 | N |
| ATOM | 1931 | CZ | ARG | B | 336 | −18.273 | 59.998 | 0.547 | 1.00 | 50.00 | C |
| ATOM | 1932 | NH1 | ARG | B | 336 | −19.095 | 59.042 | 0.927 | 1.00 | 50.00 | N |
| ATOM | 1933 | NH2 | ARG | B | 336 | −18.732 | 60.726 | −0.450 | 1.00 | 50.00 | N |
| ATOM | 1934 | N | THR | B | 337 | −12.654 | 57.888 | 5.428 | 1.00 | 50.00 | N |
| ATOM | 1935 | CA | THR | B | 337 | −11.947 | 56.932 | 6.277 | 1.00 | 50.00 | C |
| ATOM | 1936 | C | THR | B | 337 | −11.813 | 55.554 | 5.640 | 1.00 | 50.00 | C |
| ATOM | 1937 | O | THR | B | 337 | −12.525 | 55.204 | 4.699 | 1.00 | 50.00 | O |
| ATOM | 1938 | CB | THR | B | 337 | −12.646 | 56.938 | 7.635 | 1.00 | 50.00 | C |
| ATOM | 1939 | OG1 | THR | B | 337 | −13.984 | 56.462 | 7.594 | 1.00 | 50.00 | O |
| ATOM | 1940 | CG2 | THR | B | 337 | −12.550 | 58.254 | 8.405 | 1.00 | 50.00 | C |
| ATOM | 1941 | N | ASP | B | 338 | −10.888 | 54.768 | 6.164 | 1.00 | 50.00 | N |
| ATOM | 1942 | CA | ASP | B | 338 | −10.687 | 53.374 | 5.763 | 1.00 | 50.00 | C |
| ATOM | 1943 | C | ASP | B | 338 | −11.796 | 52.405 | 6.157 | 1.00 | 50.00 | C |
| ATOM | 1944 | O | ASP | B | 338 | −12.080 | 51.501 | 5.372 | 1.00 | 50.00 | O |
| ATOM | 1945 | CB | ASP | B | 338 | −9.331 | 52.869 | 6.257 | 1.00 | 50.00 | C |
| ATOM | 1946 | CG | ASP | B | 338 | −8.108 | 53.522 | 5.628 | 1.00 | 50.00 | C |
| ATOM | 1947 | OD1 | ASP | B | 338 | −7.956 | 53.458 | 4.389 | 1.00 | 50.00 | O |
| ATOM | 1948 | OD2 | ASP | B | 338 | −7.289 | 54.109 | 6.367 | 1.00 | 50.00 | O |
| ATOM | 1949 | N | ARG | B | 339 | −12.385 | 52.531 | 7.335 | 1.00 | 50.00 | N |
| ATOM | 1950 | CA | ARG | B | 339 | −13.447 | 51.661 | 7.842 | 1.00 | 50.00 | C |
| ATOM | 1951 | C | ARG | B | 339 | −14.473 | 52.543 | 8.546 | 1.00 | 50.00 | C |
| ATOM | 1952 | O | ARG | B | 339 | −14.081 | 53.466 | 9.258 | 1.00 | 50.00 | O |
| ATOM | 1953 | CB | ARG | B | 339 | −12.881 | 50.560 | 8.741 | 1.00 | 50.00 | C |
| ATOM | 1954 | CG | ARG | B | 339 | −11.853 | 49.648 | 8.072 | 1.00 | 50.00 | C |
| ATOM | 1955 | CD | ARG | B | 339 | −11.315 | 48.476 | 8.890 | 1.00 | 50.00 | C |
| ATOM | 1956 | NE | ARG | B | 339 | −10.193 | 47.853 | 8.186 | 1.00 | 50.00 | N |
| ATOM | 1957 | CZ | ARG | B | 339 | −9.443 | 46.869 | 8.654 | 1.00 | 50.00 | C |
| ATOM | 1958 | NH1 | ARG | B | 339 | −9.652 | 46.264 | 9.809 | 1.00 | 50.00 | N |
| ATOM | 1959 | NH2 | ARG | B | 339 | −8.420 | 46.463 | 7.926 | 1.00 | 50.00 | N |
| ATOM | 1960 | N | GLY | B | 340 | −15.762 | 52.301 | 8.372 | 1.00 | 50.00 | N |
| ATOM | 1961 | CA | GLY | B | 340 | −16.849 | 53.106 | 8.931 | 1.00 | 50.00 | C |
| ATOM | 1962 | C | GLY | B | 340 | −17.960 | 52.249 | 9.524 | 1.00 | 50.00 | C |
| ATOM | 1963 | O | GLY | B | 340 | −18.736 | 51.671 | 8.765 | 1.00 | 50.00 | O |
| ATOM | 1964 | N | TRP | B | 341 | −18.088 | 52.193 | 10.840 | 1.00 | 50.00 | N |
| ATOM | 1965 | CA | TRP | B | 341 | −19.190 | 51.549 | 11.556 | 1.00 | 50.00 | C |
| ATOM | 1966 | C | TRP | B | 341 | −20.361 | 52.521 | 11.614 | 1.00 | 50.00 | C |
| ATOM | 1967 | O | TRP | B | 341 | −20.180 | 53.719 | 11.828 | 1.00 | 50.00 | O |
| ATOM | 1968 | CB | TRP | B | 341 | −18.799 | 51.073 | 12.957 | 1.00 | 50.00 | C |
| ATOM | 1969 | CG | TRP | B | 341 | −19.803 | 50.249 | 13.736 | 1.00 | 50.00 | C |
| ATOM | 1970 | CD1 | TRP | B | 341 | −20.039 | 48.886 | 13.577 | 1.00 | 50.00 | C |
| ATOM | 1971 | CD2 | TRP | B | 341 | −20.698 | 50.634 | 14.729 | 1.00 | 50.00 | C |
| ATOM | 1972 | NE1 | TRP | B | 341 | −21.042 | 48.402 | 14.396 | 1.00 | 50.00 | N |
| ATOM | 1973 | CE2 | TRP | B | 341 | −21.448 | 49.515 | 15.102 | 1.00 | 50.00 | C |
| ATOM | 1974 | CE3 | TRP | B | 341 | −20.928 | 51.825 | 15.329 | 1.00 | 50.00 | C |
| ATOM | 1975 | CZ2 | TRP | B | 341 | −22.407 | 49.625 | 16.049 | 1.00 | 50.00 | C |
| ATOM | 1976 | CZ3 | TRP | B | 341 | −21.876 | 51.902 | 16.286 | 1.00 | 50.00 | C |
| ATOM | 1977 | CH2 | TRP | B | 341 | −22.599 | 50.820 | 16.643 | 1.00 | 50.00 | C |
| ATOM | 1978 | N | TYR | B | 342 | −21.560 | 51.983 | 11.461 | 1.00 | 50.00 | N |
| ATOM | 1979 | CA | TYR | B | 342 | −22.791 | 52.750 | 11.644 | 1.00 | 50.00 | C |
| ATOM | 1980 | C | TYR | B | 342 | −23.724 | 52.033 | 12.615 | 1.00 | 50.00 | C |
| ATOM | 1981 | O | TYR | B | 342 | −23.693 | 50.806 | 12.698 | 1.00 | 50.00 | O |
| ATOM | 1982 | CB | TYR | B | 342 | −23.283 | 53.309 | 10.304 | 1.00 | 50.00 | C |
| ATOM | 1983 | CG | TYR | B | 342 | −24.499 | 52.834 | 9.536 | 1.00 | 50.00 | C |
| ATOM | 1984 | CD1 | TYR | B | 342 | −24.898 | 51.552 | 9.656 | 1.00 | 50.00 | C |
| ATOM | 1985 | CD2 | TYR | B | 342 | −25.260 | 53.673 | 8.802 | 1.00 | 50.00 | C |
| ATOM | 1986 | CE1 | TYR | B | 342 | −26.130 | 51.178 | 9.255 | 1.00 | 50.00 | C |
| ATOM | 1987 | CE2 | TYR | B | 342 | −26.447 | 53.273 | 8.299 | 1.00 | 50.00 | C |
| ATOM | 1988 | CZ | TYR | B | 342 | −26.899 | 52.029 | 8.549 | 1.00 | 50.00 | C |
| ATOM | 1989 | OH | TYR | B | 342 | −28.134 | 51.577 | 8.166 | 1.00 | 50.00 | O |
| ATOM | 1990 | N | CYS | B | 343 | −24.592 | 52.732 | 13.332 | 1.00 | 50.00 | N |
| ATOM | 1991 | CA | CYS | B | 343 | −25.876 | 52.162 | 13.740 | 1.00 | 50.00 | C |
| ATOM | 1992 | C | CYS | B | 343 | −27.113 | 53.041 | 13.866 | 1.00 | 50.00 | C |
| ATOM | 1993 | O | CYS | B | 343 | −27.045 | 54.263 | 13.996 | 1.00 | 50.00 | O |
| ATOM | 1994 | CB | CYS | B | 343 | −25.790 | 51.022 | 14.755 | 1.00 | 50.00 | C |
| ATOM | 1995 | SG | CYS | B | 343 | −26.392 | 49.531 | 13.944 | 1.00 | 50.00 | S |
| ATOM | 1996 | N | ASP | B | 344 | −28.256 | 52.382 | 13.747 | 1.00 | 50.00 | N |
| ATOM | 1997 | CA | ASP | B | 344 | −29.552 | 52.998 | 13.466 | 1.00 | 50.00 | C |
| ATOM | 1998 | C | ASP | B | 344 | −30.260 | 53.239 | 14.792 | 1.00 | 50.00 | C |
| ATOM | 1999 | O | ASP | B | 344 | −30.938 | 52.370 | 15.340 | 1.00 | 50.00 | O |
| ATOM | 2000 | CB | ASP | B | 344 | −30.372 | 52.104 | 12.534 | 1.00 | 50.00 | C |
| ATOM | 2001 | CG | ASP | B | 344 | −29.828 | 51.904 | 11.126 | 1.00 | 50.00 | C |
| ATOM | 2002 | OD1 | ASP | B | 344 | −29.644 | 52.904 | 10.399 | 1.00 | 50.00 | O |
| ATOM | 2003 | OD2 | ASP | B | 344 | −29.580 | 50.743 | 10.736 | 1.00 | 50.00 | O |
| ATOM | 2004 | N | ASN | B | 345 | −30.069 | 54.443 | 15.304 | 1.00 | 50.00 | N |
| ATOM | 2005 | CA | ASN | B | 345 | −30.714 | 54.930 | 16.524 | 1.00 | 50.00 | C |
| ATOM | 2006 | C | ASN | B | 345 | −31.998 | 55.695 | 16.218 | 1.00 | 50.00 | C |
| ATOM | 2007 | O | ASN | B | 345 | −33.016 | 55.436 | 16.858 | 1.00 | 50.00 | O |
| ATOM | 2008 | CB | ASN | B | 345 | −29.702 | 55.697 | 17.378 | 1.00 | 50.00 | C |
| ATOM | 2009 | CG | ASN | B | 345 | −28.522 | 54.877 | 17.883 | 1.00 | 50.00 | C |
| ATOM | 2010 | OD1 | ASN | B | 345 | −28.673 | 53.988 | 18.719 | 1.00 | 50.00 | O |
| ATOM | 2011 | ND2 | ASN | B | 345 | −27.327 | 55.107 | 17.370 | 1.00 | 50.00 | N |
| ATOM | 2012 | N | ALA | B | 346 | −31.986 | 56.596 | 15.248 | 1.00 | 50.00 | N |
| ATOM | 2013 | CA | ALA | B | 346 | −33.173 | 57.137 | 14.586 | 1.00 | 50.00 | C |
| ATOM | 2014 | C | ALA | B | 346 | −32.774 | 57.536 | 13.170 | 1.00 | 50.00 | C |
| ATOM | 2015 | O | ALA | B | 346 | −33.188 | 56.873 | 12.219 | 1.00 | 50.00 | O |
| ATOM | 2016 | CB | ALA | B | 346 | −33.835 | 58.245 | 15.403 | 1.00 | 50.00 | C |
| ATOM | 2017 | N | GLY | B | 347 | −31.899 | 58.517 | 13.009 | 1.00 | 50.00 | N |
| ATOM | 2018 | CA | GLY | B | 347 | −30.946 | 58.544 | 11.900 | 1.00 | 50.00 | C |
| ATOM | 2019 | C | GLY | B | 347 | −29.836 | 57.503 | 11.976 | 1.00 | 50.00 | C |
| ATOM | 2020 | O | GLY | B | 347 | −29.769 | 56.661 | 12.871 | 1.00 | 50.00 | O |
| ATOM | 2021 | N | SER | B | 348 | −28.952 | 57.582 | 10.996 | 1.00 | 50.00 | N |
| ATOM | 2022 | CA | SER | B | 348 | −27.808 | 56.681 | 10.864 | 1.00 | 50.00 | C |
| ATOM | 2023 | C | SER | B | 348 | −26.567 | 57.345 | 11.450 | 1.00 | 50.00 | C |
| ATOM | 2024 | O | SER | B | 348 | −26.116 | 58.389 | 10.980 | 1.00 | 50.00 | O |
| ATOM | 2025 | CB | SER | B | 348 | −27.631 | 56.355 | 9.382 | 1.00 | 50.00 | C |
| ATOM | 2026 | OG | SER | B | 348 | −28.649 | 55.475 | 8.927 | 1.00 | 50.00 | O |
| ATOM | 2027 | N | VAL | B | 349 | −26.030 | 56.732 | 12.492 | 1.00 | 50.00 | N |
| ATOM | 2028 | CA | VAL | B | 349 | −24.940 | 57.279 | 13.300 | 1.00 | 50.00 | C |
| ATOM | 2029 | C | VAL | B | 349 | −23.659 | 56.633 | 12.786 | 1.00 | 50.00 | C |
| ATOM | 2030 | O | VAL | B | 349 | −23.514 | 55.424 | 12.953 | 1.00 | 50.00 | O |
| ATOM | 2031 | CB | VAL | B | 349 | −25.264 | 57.042 | 14.778 | 1.00 | 50.00 | C |
| ATOM | 2032 | CG1 | VAL | B | 349 | −24.066 | 57.188 | 15.714 | 1.00 | 50.00 | C |
| ATOM | 2033 | CG2 | VAL | B | 349 | −26.347 | 57.998 | 15.279 | 1.00 | 50.00 | C |
| ATOM | 2034 | N | SER | B | 350 | −22.754 | 57.385 | 12.181 | 1.00 | 50.00 | N |
| ATOM | 2035 | CA | SER | B | 350 | −21.753 | 56.853 | 11.255 | 1.00 | 50.00 | C |
| ATOM | 2036 | C | SER | B | 350 | −20.360 | 57.340 | 11.638 | 1.00 | 50.00 | C |
| ATOM | 2037 | O | SER | B | 350 | −19.896 | 58.375 | 11.162 | 1.00 | 50.00 | O |
| ATOM | 2038 | CB | SER | B | 350 | −22.163 | 57.239 | 9.834 | 1.00 | 50.00 | C |
| ATOM | 2039 | OG | SER | B | 350 | −23.340 | 56.562 | 9.418 | 1.00 | 50.00 | O |
| ATOM | 2040 | N | PHE | B | 351 | −19.673 | 56.603 | 12.495 | 1.00 | 50.00 | N |
| ATOM | 2041 | CA | PHE | B | 351 | −18.385 | 56.993 | 13.071 | 1.00 | 50.00 | C |
| ATOM | 2042 | C | PHE | B | 351 | −17.209 | 56.181 | 12.535 | 1.00 | 50.00 | C |
| ATOM | 2043 | O | PHE | B | 351 | −17.424 | 55.163 | 11.877 | 1.00 | 50.00 | O |
| ATOM | 2044 | CB | PHE | B | 351 | −18.552 | 56.967 | 14.593 | 1.00 | 50.00 | C |
| ATOM | 2045 | CG | PHE | B | 351 | −19.437 | 58.031 | 15.210 | 1.00 | 50.00 | C |
| ATOM | 2046 | CD1 | PHE | B | 351 | −19.109 | 59.322 | 14.997 | 1.00 | 50.00 | C |
| ATOM | 2047 | CD2 | PHE | B | 351 | −20.529 | 57.749 | 15.950 | 1.00 | 50.00 | C |
| ATOM | 2048 | CE1 | PHE | B | 351 | −19.854 | 60.321 | 15.511 | 1.00 | 50.00 | C |
| ATOM | 2049 | CE2 | PHE | B | 351 | −21.260 | 58.744 | 16.495 | 1.00 | 50.00 | C |
| ATOM | 2050 | CZ | PHE | B | 351 | −20.937 | 60.031 | 16.258 | 1.00 | 50.00 | C |
| ATOM | 2051 | N | PHE | B | 352 | −15.976 | 56.606 | 12.767 | 1.00 | 50.00 | N |
| ATOM | 2052 | CA | PHE | B | 352 | −14.758 | 55.914 | 12.339 | 1.00 | 50.00 | C |
| ATOM | 2053 | C | PHE | B | 352 | −14.373 | 54.842 | 13.353 | 1.00 | 50.00 | C |
| ATOM | 2054 | O | PHE | B | 352 | −13.743 | 55.222 | 14.338 | 1.00 | 50.00 | O |
| ATOM | 2055 | CB | PHE | B | 352 | −13.515 | 56.768 | 12.070 | 1.00 | 50.00 | C |
| ATOM | 2056 | CG | PHE | B | 352 | −12.223 | 56.071 | 11.696 | 1.00 | 50.00 | C |
| ATOM | 2057 | CD1 | PHE | B | 352 | −12.211 | 55.388 | 10.534 | 1.00 | 50.00 | C |
| ATOM | 2058 | CD2 | PHE | B | 352 | −11.115 | 56.066 | 12.467 | 1.00 | 50.00 | C |
| ATOM | 2059 | CE1 | PHE | B | 352 | −11.107 | 54.734 | 10.120 | 1.00 | 50.00 | C |
| ATOM | 2060 | CE2 | PHE | B | 352 | −10.006 | 55.416 | 12.061 | 1.00 | 50.00 | C |
| ATOM | 2061 | CZ | PHE | B | 352 | −9.999 | 54.755 | 10.886 | 1.00 | 50.00 | C |
| ATOM | 2062 | N | PRO | B | 353 | −14.539 | 53.546 | 13.142 | 1.00 | 50.00 | N |
| ATOM | 2063 | CA | PRO | B | 353 | −13.960 | 52.479 | 13.955 | 1.00 | 50.00 | C |
| ATOM | 2064 | C | PRO | B | 353 | −12.451 | 52.383 | 13.760 | 1.00 | 50.00 | C |
| ATOM | 2065 | O | PRO | B | 353 | −11.913 | 52.244 | 12.662 | 1.00 | 50.00 | O |
| ATOM | 2066 | CB | PRO | B | 353 | −14.687 | 51.227 | 13.465 | 1.00 | 50.00 | C |
| ATOM | 2067 | CG | PRO | B | 353 | −14.986 | 51.512 | 11.996 | 1.00 | 50.00 | C |
| ATOM | 2068 | CD | PRO | B | 353 | −15.394 | 52.978 | 12.105 | 1.00 | 50.00 | C |
| ATOM | 2069 | N | GLN | B | 354 | −11.798 | 52.415 | 14.907 | 1.00 | 50.00 | N |
| ATOM | 2070 | CA | GLN | B | 354 | −10.420 | 51.972 | 15.108 | 1.00 | 50.00 | C |
| ATOM | 2071 | C | GLN | B | 354 | −10.439 | 50.456 | 15.272 | 1.00 | 50.00 | C |
| ATOM | 2072 | O | GLN | B | 354 | −10.543 | 49.923 | 16.376 | 1.00 | 50.00 | O |
| ATOM | 2073 | CB | GLN | B | 354 | −9.851 | 52.707 | 16.323 | 1.00 | 50.00 | C |
| ATOM | 2074 | CG | GLN | B | 354 | −9.697 | 54.222 | 16.190 | 1.00 | 50.00 | C |
| ATOM | 2075 | CD | GLN | B | 354 | −8.559 | 54.701 | 15.300 | 1.00 | 50.00 | C |
| ATOM | 2076 | OE1 | GLN | B | 354 | −7.816 | 53.932 | 14.692 | 1.00 | 50.00 | O |
| ATOM | 2077 | NE2 | GLN | B | 354 | −8.394 | 56.004 | 15.171 | 1.00 | 50.00 | N |
| ATOM | 2078 | N | ALA | B | 355 | −10.331 | 49.771 | 14.145 | 1.00 | 50.00 | N |
| ATOM | 2079 | CA | ALA | B | 355 | −10.171 | 48.318 | 14.078 | 1.00 | 50.00 | C |
| ATOM | 2080 | C | ALA | B | 355 | −8.898 | 47.684 | 14.630 | 1.00 | 50.00 | C |
| ATOM | 2081 | O | ALA | B | 355 | −8.819 | 46.456 | 14.651 | 1.00 | 50.00 | O |
| ATOM | 2082 | CB | ALA | B | 355 | −10.319 | 47.936 | 12.607 | 1.00 | 50.00 | C |
| ATOM | 2083 | N | GLU | B | 356 | −7.941 | 48.455 | 15.121 | 1.00 | 50.00 | N |
| ATOM | 2084 | CA | GLU | B | 356 | −6.809 | 48.001 | 15.930 | 1.00 | 50.00 | C |
| ATOM | 2085 | C | GLU | B | 356 | −7.082 | 47.029 | 17.074 | 1.00 | 50.00 | C |
| ATOM | 2086 | O | GLU | B | 356 | −6.252 | 46.154 | 17.313 | 1.00 | 50.00 | O |
| ATOM | 2087 | CB | GLU | B | 356 | −6.051 | 49.228 | 16.442 | 1.00 | 50.00 | C |
| ATOM | 2088 | CG | GLU | B | 356 | −5.373 | 50.123 | 15.405 | 1.00 | 50.00 | C |
| ATOM | 2089 | CD | GLU | B | 356 | −4.215 | 49.461 | 14.672 | 1.00 | 50.00 | C |
| ATOM | 2090 | OE1 | GLU | B | 356 | −4.449 | 48.827 | 13.620 | 1.00 | 50.00 | O |
| ATOM | 2091 | OE2 | GLU | B | 356 | −3.063 | 49.560 | 15.147 | 1.00 | 50.00 | O |
| ATOM | 2092 | N | THR | B | 357 | −8.221 | 47.129 | 17.742 | 1.00 | 50.00 | N |
| ATOM | 2093 | CA | THR | B | 357 | −8.676 | 46.155 | 18.734 | 1.00 | 50.00 | C |
| ATOM | 2094 | C | THR | B | 357 | −9.471 | 44.985 | 18.159 | 1.00 | 50.00 | C |
| ATOM | 2095 | O | THR | B | 357 | −10.354 | 44.470 | 18.844 | 1.00 | 50.00 | O |
| ATOM | 2096 | CB | THR | B | 357 | −9.385 | 46.977 | 19.814 | 1.00 | 50.00 | C |
| ATOM | 2097 | OG1 | THR | B | 357 | −8.550 | 48.009 | 20.321 | 1.00 | 50.00 | O |
| ATOM | 2098 | CG2 | THR | B | 357 | −9.785 | 46.183 | 21.055 | 1.00 | 50.00 | C |
| ATOM | 2099 | N | CYS | B | 358 | −9.204 | 44.476 | 16.966 | 1.00 | 50.00 | N |
| ATOM | 2100 | CA | CYS | B | 358 | −10.005 | 43.411 | 16.362 | 1.00 | 50.00 | C |
| ATOM | 2101 | C | CYS | B | 358 | −9.197 | 42.480 | 15.465 | 1.00 | 50.00 | C |
| ATOM | 2102 | O | CYS | B | 358 | −8.529 | 42.928 | 14.534 | 1.00 | 50.00 | O |
| ATOM | 2103 | CB | CYS | B | 358 | −11.200 | 43.995 | 15.609 | 1.00 | 50.00 | C |
| ATOM | 2104 | SG | CYS | B | 358 | −12.091 | 45.307 | 16.466 | 1.00 | 50.00 | S |
| ATOM | 2105 | N | LYS | B | 359 | −9.331 | 41.179 | 15.675 | 1.00 | 50.00 | N |
| ATOM | 2106 | CA | LYS | B | 359 | −9.084 | 40.139 | 14.674 | 1.00 | 50.00 | C |
| ATOM | 2107 | C | LYS | B | 359 | −10.266 | 40.129 | 13.711 | 1.00 | 50.00 | C |
| ATOM | 2108 | O | LYS | B | 359 | −11.426 | 40.078 | 14.118 | 1.00 | 50.00 | O |
| ATOM | 2109 | CB | LYS | B | 359 | −8.866 | 38.712 | 15.184 | 1.00 | 50.00 | C |
| ATOM | 2110 | CG | LYS | B | 359 | −7.889 | 38.484 | 16.338 | 1.00 | 50.00 | C |
| ATOM | 2111 | CD | LYS | B | 359 | −8.065 | 37.179 | 17.115 | 1.00 | 50.00 | C |
| ATOM | 2112 | CE | LYS | B | 359 | −7.297 | 37.150 | 18.435 | 1.00 | 50.00 | C |
| ATOM | 2113 | NZ | LYS | B | 359 | −7.543 | 35.929 | 19.217 | 1.00 | 50.00 | N |
| ATOM | 2114 | N | VAL | B | 360 | −9.958 | 40.217 | 12.428 | 1.00 | 50.00 | N |
| ATOM | 2115 | CA | VAL | B | 360 | −10.934 | 40.495 | 11.374 | 1.00 | 50.00 | C |
| ATOM | 2116 | C | VAL | B | 360 | −10.961 | 39.257 | 10.488 | 1.00 | 50.00 | C |
| ATOM | 2117 | O | VAL | B | 360 | −10.008 | 38.936 | 9.778 | 1.00 | 50.00 | O |
| ATOM | 2118 | CB | VAL | B | 360 | −10.625 | 41.788 | 10.615 | 1.00 | 50.00 | C |
| ATOM | 2119 | CG1 | VAL | B | 360 | −11.505 | 42.002 | 9.383 | 1.00 | 50.00 | C |
| ATOM | 2120 | CG2 | VAL | B | 360 | −10.651 | 43.021 | 11.519 | 1.00 | 50.00 | C |
| ATOM | 2121 | N | GLN | B | 361 | −12.131 | 38.643 | 10.462 | 1.00 | 50.00 | N |
| ATOM | 2122 | CA | GLN | B | 361 | −12.540 | 37.706 | 9.417 | 1.00 | 50.00 | C |
| ATOM | 2123 | C | GLN | B | 361 | −13.161 | 38.332 | 8.175 | 1.00 | 50.00 | C |
| ATOM | 2124 | O | GLN | B | 361 | −13.525 | 39.507 | 8.139 | 1.00 | 50.00 | O |
| ATOM | 2125 | CB | GLN | B | 361 | −13.495 | 36.708 | 10.072 | 1.00 | 50.00 | C |
| ATOM | 2126 | CG | GLN | B | 361 | −12.823 | 35.690 | 10.985 | 1.00 | 50.00 | C |
| ATOM | 2127 | CD | GLN | B | 361 | −11.684 | 34.964 | 10.288 | 1.00 | 50.00 | C |
| ATOM | 2128 | OE1 | GLN | B | 361 | −11.881 | 34.361 | 9.234 | 1.00 | 50.00 | O |
| ATOM | 2129 | NE2 | GLN | B | 361 | −10.468 | 35.195 | 10.749 | 1.00 | 50.00 | N |
| ATOM | 2130 | N | SER | B | 362 | −13.322 | 37.482 | 7.174 | 1.00 | 50.00 | N |
| ATOM | 2131 | CA | SER | B | 362 | −14.148 | 37.740 | 5.994 | 1.00 | 50.00 | C |
| ATOM | 2132 | C | SER | B | 362 | −15.567 | 38.193 | 6.322 | 1.00 | 50.00 | C |
| ATOM | 2133 | O | SER | B | 362 | −15.923 | 39.329 | 6.011 | 1.00 | 50.00 | O |
| ATOM | 2134 | CB | SER | B | 362 | −14.124 | 36.506 | 5.092 | 1.00 | 50.00 | C |
| ATOM | 2135 | OG | SER | B | 362 | −12.818 | 36.200 | 4.624 | 1.00 | 50.00 | O |
| ATOM | 2136 | N | ASN | B | 363 | −16.324 | 37.353 | 7.009 | 1.00 | 50.00 | N |
| ATOM | 2137 | CA | ASN | B | 363 | −17.624 | 37.677 | 7.598 | 1.00 | 50.00 | C |
| ATOM | 2138 | C | ASN | B | 363 | −17.641 | 37.715 | 9.124 | 1.00 | 50.00 | C |
| ATOM | 2139 | O | ASN | B | 363 | −18.761 | 37.701 | 9.634 | 1.00 | 50.00 | O |
| ATOM | 2140 | CB | ASN | B | 363 | −18.724 | 36.730 | 7.112 | 1.00 | 50.00 | C |
| ATOM | 2141 | CG | ASN | B | 363 | −18.744 | 36.219 | 5.680 | 1.00 | 50.00 | C |
| ATOM | 2142 | OD1 | ASN | B | 363 | −17.984 | 35.312 | 5.345 | 1.00 | 50.00 | O |
| ATOM | 2143 | ND2 | ASN | B | 363 | −19.595 | 36.774 | 4.838 | 1.00 | 50.00 | N |
| ATOM | 2144 | N | ARG | B | 364 | −16.565 | 37.774 | 9.897 | 1.00 | 50.00 | N |
| ATOM | 2145 | CA | ARG | B | 364 | −16.530 | 38.265 | 11.279 | 1.00 | 50.00 | C |
| ATOM | 2146 | C | ARG | B | 364 | −15.525 | 39.380 | 11.564 | 1.00 | 50.00 | C |
| ATOM | 2147 | O | ARG | B | 364 | −14.676 | 39.715 | 10.739 | 1.00 | 50.00 | O |
| ATOM | 2148 | CB | ARG | B | 364 | −16.893 | 37.347 | 12.448 | 1.00 | 50.00 | C |
| ATOM | 2149 | CG | ARG | B | 364 | −18.258 | 36.676 | 12.317 | 1.00 | 50.00 | C |
| ATOM | 2150 | CD | ARG | B | 364 | −18.782 | 36.018 | 13.591 | 1.00 | 50.00 | C |
| ATOM | 2151 | NE | ARG | B | 364 | −19.957 | 35.182 | 13.358 | 1.00 | 50.00 | N |
| ATOM | 2152 | CZ | ARG | B | 364 | −20.327 | 34.192 | 14.151 | 1.00 | 50.00 | C |
| ATOM | 2153 | NH1 | ARG | B | 364 | −20.058 | 34.095 | 15.437 | 1.00 | 50.00 | N |
| ATOM | 2154 | NH2 | ARG | B | 364 | −21.030 | 33.207 | 13.634 | 1.00 | 50.00 | N |
| ATOM | 2155 | N | VAL | B | 365 | −15.673 | 40.080 | 12.677 | 1.00 | 50.00 | N |
| ATOM | 2156 | CA | VAL | B | 365 | −14.726 | 41.024 | 13.272 | 1.00 | 50.00 | C |
| ATOM | 2157 | C | VAL | B | 365 | −15.032 | 40.758 | 14.741 | 1.00 | 50.00 | C |
| ATOM | 2158 | O | VAL | B | 365 | −15.976 | 41.286 | 15.330 | 1.00 | 50.00 | O |
| ATOM | 2159 | CB | VAL | B | 365 | −14.785 | 42.521 | 12.950 | 1.00 | 50.00 | C |
| ATOM | 2160 | CG1 | VAL | B | 365 | −14.010 | 43.489 | 13.840 | 1.00 | 50.00 | C |
| ATOM | 2161 | CG2 | VAL | B | 365 | −14.407 | 42.913 | 11.523 | 1.00 | 50.00 | C |
| ATOM | 2162 | N | PHE | B | 366 | −14.145 | 39.975 | 15.329 | 1.00 | 50.00 | N |
| ATOM | 2163 | CA | PHE | B | 366 | −14.002 | 39.846 | 16.777 | 1.00 | 50.00 | C |
| ATOM | 2164 | C | PHE | B | 366 | −13.424 | 41.148 | 17.317 | 1.00 | 50.00 | C |
| ATOM | 2165 | O | PHE | B | 366 | −12.321 | 41.514 | 16.916 | 1.00 | 50.00 | O |
| ATOM | 2166 | CB | PHE | B | 366 | −13.245 | 38.537 | 17.026 | 1.00 | 50.00 | C |
| ATOM | 2167 | CG | PHE | B | 366 | −11.939 | 38.242 | 17.736 | 1.00 | 50.00 | C |
| ATOM | 2168 | CD1 | PHE | B | 366 | −10.997 | 39.199 | 17.648 | 1.00 | 50.00 | C |
| ATOM | 2169 | CD2 | PHE | B | 366 | −11.798 | 37.309 | 18.701 | 1.00 | 50.00 | C |
| ATOM | 2170 | CE1 | PHE | B | 366 | −10.160 | 39.443 | 18.674 | 1.00 | 50.00 | C |
| ATOM | 2171 | CE2 | PHE | B | 366 | −10.937 | 37.504 | 19.721 | 1.00 | 50.00 | C |
| ATOM | 2172 | CZ | PHE | B | 366 | −10.149 | 38.599 | 19.725 | 1.00 | 50.00 | C |
| ATOM | 2173 | N | CYS | B | 367 | −14.111 | 41.918 | 18.143 | 1.00 | 50.00 | N |
| ATOM | 2174 | CA | CYS | B | 367 | −13.486 | 43.023 | 18.870 | 1.00 | 50.00 | C |
| ATOM | 2175 | C | CYS | B | 367 | −13.394 | 42.698 | 20.355 | 1.00 | 50.00 | C |
| ATOM | 2176 | O | CYS | B | 367 | −14.277 | 42.086 | 20.953 | 1.00 | 50.00 | O |
| ATOM | 2177 | CB | CYS | B | 367 | −14.188 | 44.351 | 18.600 | 1.00 | 50.00 | C |
| ATOM | 2178 | SG | CYS | B | 367 | −13.988 | 44.814 | 16.873 | 1.00 | 50.00 | S |
| ATOM | 2179 | N | ASP | B | 368 | −12.317 | 43.148 | 20.975 | 1.00 | 50.00 | N |
| ATOM | 2180 | CA | ASP | B | 368 | −12.230 | 43.306 | 22.428 | 1.00 | 50.00 | C |
| ATOM | 2181 | C | ASP | B | 368 | −12.881 | 44.569 | 22.986 | 1.00 | 50.00 | C |
| ATOM | 2182 | O | ASP | B | 368 | −13.345 | 44.551 | 24.126 | 1.00 | 50.00 | O |
| ATOM | 2183 | CB | ASP | B | 368 | −10.755 | 43.165 | 22.809 | 1.00 | 50.00 | C |
| ATOM | 2184 | CG | ASP | B | 368 | −10.090 | 41.822 | 22.540 | 1.00 | 50.00 | C |
| ATOM | 2185 | OD1 | ASP | B | 368 | −10.522 | 40.797 | 23.111 | 1.00 | 50.00 | O |
| ATOM | 2186 | OD2 | ASP | B | 368 | −9.137 | 41.777 | 21.732 | 1.00 | 50.00 | O |
| ATOM | 2187 | N | THR | B | 369 | −12.922 | 45.655 | 22.230 | 1.00 | 50.00 | N |
| ATOM | 2188 | CA | THR | B | 369 | −13.467 | 46.969 | 22.575 | 1.00 | 50.00 | C |
| ATOM | 2189 | C | THR | B | 369 | −13.958 | 47.608 | 21.279 | 1.00 | 50.00 | C |
| ATOM | 2190 | O | THR | B | 369 | −13.547 | 47.251 | 20.176 | 1.00 | 50.00 | O |
| ATOM | 2191 | CB | THR | B | 369 | −12.388 | 47.822 | 23.247 | 1.00 | 50.00 | C |
| ATOM | 2192 | OG1 | THR | B | 369 | −11.864 | 47.176 | 24.398 | 1.00 | 50.00 | O |
| ATOM | 2193 | CG2 | THR | B | 369 | −12.767 | 49.231 | 23.696 | 1.00 | 50.00 | C |
| ATOM | 2194 | N | MET | B | 370 | −14.807 | 48.619 | 21.376 | 1.00 | 50.00 | N |
| ATOM | 2195 | CA | MET | B | 370 | −15.299 | 49.362 | 20.214 | 1.00 | 50.00 | C |
| ATOM | 2196 | C | MET | B | 370 | −14.770 | 50.789 | 20.194 | 1.00 | 50.00 | C |
| ATOM | 2197 | O | MET | B | 370 | −15.437 | 51.751 | 20.573 | 1.00 | 50.00 | O |
| ATOM | 2198 | CB | MET | B | 370 | −16.821 | 49.251 | 20.150 | 1.00 | 50.00 | C |
| ATOM | 2199 | CG | MET | B | 370 | −17.247 | 47.856 | 19.699 | 1.00 | 50.00 | C |
| ATOM | 2200 | SD | MET | B | 370 | −16.589 | 47.416 | 18.081 | 1.00 | 50.00 | S |
| ATOM | 2201 | CE | MET | B | 370 | −17.567 | 48.512 | 17.041 | 1.00 | 50.00 | C |
| ATOM | 2202 | N | ASN | B | 371 | −13.552 | 50.891 | 19.689 | 1.00 | 50.00 | N |
| ATOM | 2203 | CA | ASN | B | 371 | −12.795 | 52.139 | 19.615 | 1.00 | 50.00 | C |
| ATOM | 2204 | C | ASN | B | 371 | −13.205 | 52.925 | 18.369 | 1.00 | 50.00 | C |
| ATOM | 2205 | O | ASN | B | 371 | −13.004 | 52.376 | 17.289 | 1.00 | 50.00 | O |
| ATOM | 2206 | CB | ASN | B | 371 | −11.335 | 51.737 | 19.860 | 1.00 | 50.00 | C |
| ATOM | 2207 | CG | ASN | B | 371 | −10.901 | 51.296 | 21.255 | 1.00 | 50.00 | C |
| ATOM | 2208 | OD1 | ASN | B | 371 | −11.618 | 51.467 | 22.239 | 1.00 | 50.00 | O |
| ATOM | 2209 | ND2 | ASN | B | 371 | −9.722 | 50.751 | 21.491 | 1.00 | 50.00 | N |
| ATOM | 2210 | N | SER | B | 372 | −13.853 | 54.081 | 18.430 | 1.00 | 50.00 | N |
| ATOM | 2211 | CA | SER | B | 372 | −14.355 | 54.906 | 17.325 | 1.00 | 50.00 | C |
| ATOM | 2212 | C | SER | B | 372 | −14.387 | 56.432 | 17.447 | 1.00 | 50.00 | C |
| ATOM | 2213 | O | SER | B | 372 | −14.580 | 56.951 | 18.545 | 1.00 | 50.00 | O |
| ATOM | 2214 | CB | SER | B | 372 | −15.788 | 54.463 | 17.031 | 1.00 | 50.00 | C |
| ATOM | 2215 | OG | SER | B | 372 | −16.269 | 54.996 | 15.805 | 1.00 | 50.00 | O |
| ATOM | 2216 | N | LEU | B | 373 | −14.278 | 57.207 | 16.376 | 1.00 | 50.00 | N |
| ATOM | 2217 | CA | LEU | B | 373 | −14.063 | 58.659 | 16.344 | 1.00 | 50.00 | C |
| ATOM | 2218 | C | LEU | B | 373 | −15.146 | 59.444 | 15.603 | 1.00 | 50.00 | C |
| ATOM | 2219 | O | LEU | B | 373 | −15.724 | 58.911 | 14.657 | 1.00 | 50.00 | O |
| ATOM | 2220 | CB | LEU | B | 373 | −12.711 | 58.968 | 15.691 | 1.00 | 50.00 | C |
| ATOM | 2221 | CG | LEU | B | 373 | −11.410 | 58.814 | 16.481 | 1.00 | 50.00 | C |
| ATOM | 2222 | CD1 | LEU | B | 373 | −11.045 | 57.359 | 16.767 | 1.00 | 50.00 | C |
| ATOM | 2223 | CD2 | LEU | B | 373 | −10.183 | 59.383 | 15.768 | 1.00 | 50.00 | C |
| ATOM | 2224 | N | THR | B | 374 | −15.394 | 60.710 | 15.915 | 1.00 | 50.00 | N |
| ATOM | 2225 | CA | THR | B | 374 | −16.233 | 61.631 | 15.140 | 1.00 | 50.00 | C |
| ATOM | 2226 | C | THR | B | 374 | −15.552 | 62.215 | 13.907 | 1.00 | 50.00 | C |
| ATOM | 2227 | O | THR | B | 374 | −14.367 | 62.548 | 13.928 | 1.00 | 50.00 | O |
| ATOM | 2228 | CB | THR | B | 374 | −16.915 | 62.723 | 15.969 | 1.00 | 50.00 | C |
| ATOM | 2229 | OG1 | THR | B | 374 | −17.653 | 62.136 | 17.032 | 1.00 | 50.00 | O |
| ATOM | 2230 | CG2 | THR | B | 374 | −17.895 | 63.644 | 15.240 | 1.00 | 50.00 | C |
| ATOM | 2231 | N | LEU | B | 375 | −16.314 | 62.321 | 12.828 | 1.00 | 50.00 | N |
| ATOM | 2232 | CA | LEU | B | 375 | −15.844 | 62.716 | 11.499 | 1.00 | 50.00 | C |
| ATOM | 2233 | C | LEU | B | 375 | −16.409 | 64.088 | 11.147 | 1.00 | 50.00 | C |
| ATOM | 2234 | O | LEU | B | 375 | −17.517 | 64.392 | 11.590 | 1.00 | 50.00 | O |
| ATOM | 2235 | CB | LEU | B | 375 | −16.188 | 61.690 | 10.415 | 1.00 | 50.00 | C |
| ATOM | 2236 | CG | LEU | B | 375 | −15.377 | 60.393 | 10.415 | 1.00 | 50.00 | C |
| ATOM | 2237 | CD1 | LEU | B | 375 | −15.609 | 59.631 | 11.716 | 1.00 | 50.00 | C |
| ATOM | 2238 | CD2 | LEU | B | 375 | −15.669 | 59.391 | 9.299 | 1.00 | 50.00 | C |
| ATOM | 2239 | N | PRO | B | 376 | −15.777 | 64.911 | 10.320 | 1.00 | 50.00 | N |
| ATOM | 2240 | CA | PRO | B | 376 | −16.384 | 66.102 | 9.722 | 1.00 | 50.00 | C |
| ATOM | 2241 | C | PRO | B | 376 | −17.827 | 65.961 | 9.254 | 1.00 | 50.00 | C |
| ATOM | 2242 | O | PRO | B | 376 | −18.182 | 64.916 | 8.708 | 1.00 | 50.00 | O |
| ATOM | 2243 | CB | PRO | B | 376 | −15.467 | 66.408 | 8.538 | 1.00 | 50.00 | C |
| ATOM | 2244 | CG | PRO | B | 376 | −14.098 | 65.840 | 8.898 | 1.00 | 50.00 | C |
| ATOM | 2245 | CD | PRO | B | 376 | −14.404 | 64.700 | 9.865 | 1.00 | 50.00 | C |
| ATOM | 2246 | N | SER | B | 377 | −18.657 | 66.968 | 9.472 | 1.00 | 50.00 | N |
| ATOM | 2247 | CA | SER | B | 377 | −20.029 | 67.000 | 8.963 | 1.00 | 50.00 | C |
| ATOM | 2248 | C | SER | B | 377 | −20.168 | 66.639 | 7.489 | 1.00 | 50.00 | C |
| ATOM | 2249 | O | SER | B | 377 | −20.881 | 65.688 | 7.172 | 1.00 | 50.00 | O |
| ATOM | 2250 | CB | SER | B | 377 | −20.695 | 68.339 | 9.284 | 1.00 | 50.00 | C |
| ATOM | 2251 | OG | SER | B | 377 | −20.802 | 68.537 | 10.687 | 1.00 | 50.00 | O |
| ATOM | 2252 | N | GLU | B | 378 | −19.382 | 67.267 | 6.630 | 1.00 | 50.00 | N |
| ATOM | 2253 | CA | GLU | B | 378 | −19.183 | 66.912 | 5.224 | 1.00 | 50.00 | C |
| ATOM | 2254 | C | GLU | B | 378 | −18.982 | 65.432 | 4.922 | 1.00 | 50.00 | C |
| ATOM | 2255 | O | GLU | B | 378 | −19.845 | 64.909 | 4.221 | 1.00 | 50.00 | O |
| ATOM | 2256 | CB | GLU | B | 378 | −18.080 | 67.798 | 4.643 | 1.00 | 50.00 | C |
| ATOM | 2257 | CG | GLU | B | 378 | −18.360 | 69.300 | 4.707 | 1.00 | 50.00 | C |
| ATOM | 2258 | CD | GLU | B | 378 | −19.337 | 69.792 | 3.651 | 1.00 | 50.00 | C |
| ATOM | 2259 | OE1 | GLU | B | 378 | −20.551 | 69.863 | 3.942 | 1.00 | 50.00 | O |
| ATOM | 2260 | OE2 | GLU | B | 378 | −18.906 | 70.097 | 2.519 | 1.00 | 50.00 | O |
| ATOM | 2261 | N | VAL | B | 379 | −18.016 | 64.723 | 5.484 | 1.00 | 50.00 | N |
| ATOM | 2262 | CA | VAL | B | 379 | −17.918 | 63.262 | 5.445 | 1.00 | 50.00 | C |
| ATOM | 2263 | C | VAL | B | 379 | −19.214 | 62.502 | 5.721 | 1.00 | 50.00 | C |
| ATOM | 2264 | O | VAL | B | 379 | −19.617 | 61.699 | 4.880 | 1.00 | 50.00 | O |
| ATOM | 2265 | CB | VAL | B | 379 | −16.763 | 62.790 | 6.334 | 1.00 | 50.00 | C |
| ATOM | 2266 | CG1 | VAL | B | 379 | −16.673 | 61.277 | 6.525 | 1.00 | 50.00 | C |
| ATOM | 2267 | CG2 | VAL | B | 379 | −15.371 | 63.250 | 5.901 | 1.00 | 50.00 | C |
| ATOM | 2268 | N | ASN | B | 380 | −19.850 | 62.718 | 6.861 | 1.00 | 50.00 | N |
| ATOM | 2269 | CA | ASN | B | 380 | −21.144 | 62.131 | 7.216 | 1.00 | 50.00 | C |
| ATOM | 2270 | C | ASN | B | 380 | −22.269 | 62.368 | 6.217 | 1.00 | 50.00 | C |
| ATOM | 2271 | O | ASN | B | 380 | −22.827 | 61.397 | 5.708 | 1.00 | 50.00 | O |
| ATOM | 2272 | CB | ASN | B | 380 | −21.599 | 62.589 | 8.603 | 1.00 | 50.00 | C |
| ATOM | 2273 | CG | ASN | B | 380 | −20.813 | 62.114 | 9.816 | 1.00 | 50.00 | C |
| ATOM | 2274 | OD1 | ASN | B | 380 | −20.458 | 62.891 | 10.701 | 1.00 | 50.00 | O |
| ATOM | 2275 | ND2 | ASN | B | 380 | −20.558 | 60.822 | 9.902 | 1.00 | 50.00 | N |
| ATOM | 2276 | N | LEU | B | 381 | −22.550 | 63.618 | 5.888 | 1.00 | 50.00 | N |
| ATOM | 2277 | CA | LEU | B | 381 | −23.329 | 64.053 | 4.729 | 1.00 | 50.00 | C |
| ATOM | 2278 | C | LEU | B | 381 | −23.100 | 63.231 | 3.465 | 1.00 | 50.00 | C |
| ATOM | 2279 | O | LEU | B | 381 | −23.995 | 62.499 | 3.046 | 1.00 | 50.00 | O |
| ATOM | 2280 | CB | LEU | B | 381 | −23.034 | 65.548 | 4.582 | 1.00 | 50.00 | C |
| ATOM | 2281 | CG | LEU | B | 381 | −23.944 | 66.563 | 5.274 | 1.00 | 50.00 | C |
| ATOM | 2282 | CD1 | LEU | B | 381 | −23.934 | 66.470 | 6.799 | 1.00 | 50.00 | C |
| ATOM | 2283 | CD2 | LEU | B | 381 | −23.553 | 67.981 | 4.860 | 1.00 | 50.00 | C |
| ATOM | 2284 | N | CYS | B | 382 | −21.886 | 63.252 | 2.943 | 1.00 | 50.00 | N |
| ATOM | 2285 | CA | CYS | B | 382 | −21.362 | 62.331 | 1.935 | 1.00 | 50.00 | C |
| ATOM | 2286 | C | CYS | B | 382 | −21.654 | 60.835 | 2.031 | 1.00 | 50.00 | C |
| ATOM | 2287 | O | CYS | B | 382 | −21.783 | 60.197 | 0.988 | 1.00 | 50.00 | O |
| ATOM | 2288 | CB | CYS | B | 382 | −19.873 | 62.675 | 1.912 | 1.00 | 50.00 | C |
| ATOM | 2289 | SG | CYS | B | 382 | −19.378 | 63.205 | 0.270 | 1.00 | 50.00 | S |
| ATOM | 2290 | N | ASN | B | 383 | −21.790 | 60.247 | 3.209 | 1.00 | 50.00 | N |
| ATOM | 2291 | CA | ASN | B | 383 | −22.303 | 58.890 | 3.414 | 1.00 | 50.00 | C |
| ATOM | 2292 | C | ASN | B | 383 | −23.640 | 58.824 | 4.147 | 1.00 | 50.00 | C |
| ATOM | 2293 | O | ASN | B | 383 | −23.814 | 58.058 | 5.094 | 1.00 | 50.00 | O |
| ATOM | 2294 | CB | ASN | B | 383 | −21.237 | 58.063 | 4.137 | 1.00 | 50.00 | C |
| ATOM | 2295 | CG | ASN | B | 383 | −20.065 | 57.507 | 3.342 | 1.00 | 50.00 | C |
| ATOM | 2296 | OD1 | ASN | B | 383 | −18.906 | 57.771 | 3.659 | 1.00 | 50.00 | O |
| ATOM | 2297 | ND1 | ASN | B | 383 | −20.331 | 56.759 | 2.288 | 1.00 | 50.00 | N |
| ATOM | 2298 | N | VAL | B | 384 | −24.639 | 59.572 | 3.706 | 1.00 | 50.00 | N |
| ATOM | 2299 | CA | VAL | B | 384 | −26.007 | 59.526 | 4.224 | 1.00 | 50.00 | C |
| ATOM | 2300 | C | VAL | B | 384 | −26.854 | 59.734 | 2.974 | 1.00 | 50.00 | C |
| ATOM | 2301 | O | VAL | B | 384 | −26.769 | 60.780 | 2.331 | 1.00 | 50.00 | O |
| ATOM | 2302 | CB | VAL | B | 384 | −26.288 | 60.531 | 5.346 | 1.00 | 50.00 | C |
| ATOM | 2303 | CG1 | VAL | B | 384 | −27.766 | 60.710 | 5.691 | 1.00 | 50.00 | C |
| ATOM | 2304 | CG2 | VAL | B | 384 | −25.596 | 60.194 | 6.666 | 1.00 | 50.00 | C |
| ATOM | 2305 | N | ASP | B | 385 | −27.690 | 58.761 | 2.645 | 1.00 | 50.00 | N |
| ATOM | 2306 | CA | ASP | B | 385 | −28.593 | 58.803 | 1.493 | 1.00 | 50.00 | C |
| ATOM | 2307 | C | ASP | B | 385 | −27.831 | 58.799 | 0.170 | 1.00 | 50.00 | C |
| ATOM | 2308 | O | ASP | B | 385 | −26.741 | 58.241 | 0.046 | 1.00 | 50.00 | O |
| ATOM | 2309 | CB | ASP | B | 385 | −29.691 | 59.855 | 1.683 | 1.00 | 50.00 | C |
| ATOM | 2310 | CG | ASP | B | 385 | −30.730 | 59.633 | 2.772 | 1.00 | 50.00 | C |
| ATOM | 2311 | OD1 | ASP | B | 385 | −31.475 | 58.632 | 2.698 | 1.00 | 50.00 | O |
| ATOM | 2312 | OD2 | ASP | B | 385 | −30.834 | 60.477 | 3.687 | 1.00 | 50.00 | O |
| ATOM | 2313 | N | ILE | B | 386 | −28.401 | 59.449 | −0.831 | 1.00 | 50.00 | N |
| ATOM | 2314 | CA | ILE | B | 386 | −27.739 | 59.898 | −2.056 | 1.00 | 50.00 | C |
| ATOM | 2315 | C | ILE | B | 386 | −27.441 | 61.381 | −1.837 | 1.00 | 50.00 | C |
| ATOM | 2316 | O | ILE | B | 386 | −28.027 | 62.220 | −2.521 | 1.00 | 50.00 | O |
| ATOM | 2317 | CB | ILE | B | 386 | −28.676 | 59.494 | −3.201 | 1.00 | 50.00 | C |
| ATOM | 2318 | CG1 | ILE | B | 386 | −28.942 | 57.993 | −3.353 | 1.00 | 50.00 | C |
| ATOM | 2319 | CG2 | ILE | B | 386 | −28.273 | 60.002 | −4.585 | 1.00 | 50.00 | C |
| ATOM | 2320 | CD1 | ILE | B | 386 | −30.006 | 57.569 | −4.365 | 1.00 | 50.00 | C |
| ATOM | 2321 | N | PHE | B | 387 | −26.547 | 61.763 | −0.936 | 1.00 | 50.00 | N |
| ATOM | 2322 | CA | PHE | B | 387 | −26.079 | 63.141 | −0.757 | 1.00 | 50.00 | C |
| ATOM | 2323 | C | PHE | B | 387 | −24.748 | 63.477 | −1.423 | 1.00 | 50.00 | C |
| ATOM | 2324 | O | PHE | B | 387 | −24.659 | 64.550 | −2.020 | 1.00 | 50.00 | O |
| ATOM | 2325 | CB | PHE | B | 387 | −26.080 | 63.559 | 0.714 | 1.00 | 50.00 | C |
| ATOM | 2326 | CG | PHE | B | 387 | −25.773 | 65.019 | 0.979 | 1.00 | 50.00 | C |
| ATOM | 2327 | CD1 | PHE | B | 387 | −26.706 | 65.977 | 0.800 | 1.00 | 50.00 | C |
| ATOM | 2328 | CD2 | PHE | B | 387 | −24.507 | 65.381 | 1.269 | 1.00 | 50.00 | C |
| ATOM | 2329 | CE1 | PHE | B | 387 | −26.380 | 67.281 | 0.910 | 1.00 | 50.00 | C |
| ATOM | 2330 | CE2 | PHE | B | 387 | −24.193 | 66.681 | 1.434 | 1.00 | 50.00 | C |
| ATOM | 2331 | CZ | PHE | B | 387 | −25.120 | 67.637 | 1.230 | 1.00 | 50.00 | C |
| ATOM | 2332 | N | ASN | B | 388 | −23.721 | 62.645 | −1.355 | 1.00 | 50.00 | N |
| ATOM | 2333 | CA | ASN | B | 388 | −22.566 | 62.679 | −2.256 | 1.00 | 50.00 | C |
| ATOM | 2334 | C | ASN | B | 388 | −22.748 | 63.029 | −3.733 | 1.00 | 50.00 | C |
| ATOM | 2335 | O | ASN | B | 388 | −21.886 | 63.750 | −4.234 | 1.00 | 50.00 | O |
| ATOM | 2336 | CB | ASN | B | 388 | −21.746 | 61.405 | −2.043 | 1.00 | 50.00 | C |
| ATOM | 2337 | CG | ASN | B | 388 | −22.363 | 60.059 | −2.400 | 1.00 | 50.00 | C |
| ATOM | 2338 | OD1 | ASN | B | 388 | −22.247 | 59.625 | −3.545 | 1.00 | 50.00 | O |
| ATOM | 2339 | ND2 | ASN | B | 388 | −23.135 | 59.418 | −1.541 | 1.00 | 50.00 | N |
| ATOM | 2340 | N | PRO | B | 389 | −23.778 | 62.634 | −4.468 | 1.00 | 50.00 | N |
| ATOM | 2341 | CA | PRO | B | 389 | −24.071 | 63.041 | −5.846 | 1.00 | 50.00 | C |
| ATOM | 2342 | C | PRO | B | 389 | −24.562 | 64.455 | −6.121 | 1.00 | 50.00 | C |
| ATOM | 2343 | O | PRO | B | 389 | −24.250 | 65.045 | −7.155 | 1.00 | 50.00 | O |
| ATOM | 2344 | CB | PRO | B | 389 | −25.254 | 62.190 | −6.306 | 1.00 | 50.00 | C |
| ATOM | 2345 | CG | PRO | B | 389 | −25.039 | 61.020 | −5.357 | 1.00 | 50.00 | C |
| ATOM | 2346 | CD | PRO | B | 389 | −24.788 | 61.682 | −4.012 | 1.00 | 50.00 | C |
| ATOM | 2347 | N | LYS | B | 390 | −25.360 | 64.942 | −5.186 | 1.00 | 50.00 | N |
| ATOM | 2348 | CA | LYS | B | 390 | −25.766 | 66.342 | −5.096 | 1.00 | 50.00 | C |
| ATOM | 2349 | C | LYS | B | 390 | −24.646 | 67.355 | −4.887 | 1.00 | 50.00 | C |
| ATOM | 2350 | O | LYS | B | 390 | −25.010 | 68.528 | −4.816 | 1.00 | 50.00 | O |
| ATOM | 2351 | CB | LYS | B | 390 | −26.805 | 66.417 | −3.977 | 1.00 | 50.00 | C |
| ATOM | 2352 | CG | LYS | B | 390 | −28.093 | 65.635 | −4.227 | 1.00 | 50.00 | C |
| ATOM | 2353 | CD | LYS | B | 390 | −29.149 | 65.933 | −3.165 | 1.00 | 50.00 | C |
| ATOM | 2354 | CE | LYS | B | 390 | −30.433 | 65.145 | −3.411 | 1.00 | 50.00 | C |
| ATOM | 2355 | NZ | LYS | B | 390 | −31.446 | 65.531 | −2.419 | 1.00 | 50.00 | N |
| ATOM | 2356 | N | TYR | B | 391 | −23.385 | 66.950 | −4.845 | 1.00 | 50.00 | N |
| ATOM | 2357 | CA | TYR | B | 391 | −22.161 | 67.751 | −4.883 | 1.00 | 50.00 | C |
| ATOM | 2358 | C | TYR | B | 391 | −21.156 | 67.440 | −3.779 | 1.00 | 50.00 | C |
| ATOM | 2359 | O | TYR | B | 391 | −19.997 | 67.811 | −3.968 | 1.00 | 50.00 | O |
| ATOM | 2360 | CB | TYR | B | 391 | −22.359 | 69.240 | −5.175 | 1.00 | 50.00 | C |
| ATOM | 2361 | CG | TYR | B | 391 | −21.177 | 70.183 | −5.184 | 1.00 | 50.00 | C |
| ATOM | 2362 | CD1 | TYR | B | 391 | −20.319 | 70.232 | −6.224 | 1.00 | 50.00 | C |
| ATOM | 2363 | CD2 | TYR | B | 391 | −20.932 | 70.905 | −4.072 | 1.00 | 50.00 | C |
| ATOM | 2364 | CE1 | TYR | B | 391 | −19.213 | 71.000 | −6.146 | 1.00 | 50.00 | C |
| ATOM | 2365 | CE2 | TYR | B | 391 | −19.830 | 71.676 | −3.995 | 1.00 | 50.00 | C |
| ATOM | 2366 | CZ | TYR | B | 391 | −18.968 | 71.718 | −5.031 | 1.00 | 50.00 | C |
| ATOM | 2367 | OH | TYR | B | 391 | −17.815 | 72.447 | −4.905 | 1.00 | 50.00 | O |
| ATOM | 2368 | N | ASP | B | 392 | −21.475 | 66.809 | −2.660 | 1.00 | 50.00 | N |
| ATOM | 2369 | CA | ASP | B | 392 | −20.571 | 66.744 | −1.511 | 1.00 | 50.00 | C |
| ATOM | 2370 | C | ASP | B | 392 | −19.339 | 65.879 | −1.755 | 1.00 | 50.00 | C |
| ATOM | 2371 | O | ASP | B | 392 | −19.045 | 65.409 | −2.853 | 1.00 | 50.00 | O |
| ATOM | 2372 | CB | ASP | B | 392 | −21.389 | 66.456 | −0.252 | 1.00 | 50.00 | C |
| ATOM | 2373 | CG | ASP | B | 392 | −20.770 | 66.938 | 1.053 | 1.00 | 50.00 | C |
| ATOM | 2374 | OD1 | ASP | B | 392 | −20.800 | 68.159 | 1.321 | 1.00 | 50.00 | O |
| ATOM | 2375 | OD2 | ASP | B | 392 | −20.255 | 66.104 | 1.827 | 1.00 | 50.00 | O |
| ATOM | 2376 | N | CYS | B | 393 | −18.547 | 65.793 | −0.701 | 1.00 | 50.00 | N |
| ATOM | 2377 | CA | CYS | B | 393 | −17.195 | 65.245 | −0.678 | 1.00 | 50.00 | C |
| ATOM | 2378 | C | CYS | B | 393 | −16.098 | 66.246 | −0.997 | 1.00 | 50.00 | C |
| ATOM | 2379 | O | CYS | B | 393 | −16.254 | 67.183 | −1.779 | 1.00 | 50.00 | O |
| ATOM | 2380 | CB | CYS | B | 393 | −16.962 | 63.864 | −1.288 | 1.00 | 50.00 | C |
| ATOM | 2381 | SG | CYS | B | 393 | −17.400 | 62.924 | 0.176 | 1.00 | 50.00 | S |
| ATOM | 2382 | N | LYS | B | 394 | −15.007 | 65.977 | −0.301 | 1.00 | 50.00 | N |
| ATOM | 2383 | CA | LYS | B | 394 | −13.816 | 66.819 | −0.356 | 1.00 | 50.00 | C |
| ATOM | 2384 | C | LYS | B | 394 | −12.689 | 65.940 | −0.874 | 1.00 | 50.00 | C |
| ATOM | 2385 | O | LYS | B | 394 | −11.930 | 65.260 | −0.185 | 1.00 | 50.00 | O |
| ATOM | 2386 | CB | LYS | B | 394 | −13.517 | 67.496 | 0.978 | 1.00 | 50.00 | C |
| ATOM | 2387 | CG | LYS | B | 394 | −14.630 | 68.490 | 1.302 | 1.00 | 50.00 | C |
| ATOM | 2388 | CD | LYS | B | 394 | −14.238 | 69.349 | 2.499 | 1.00 | 50.00 | C |
| ATOM | 2389 | CE | LYS | B | 394 | −15.393 | 70.297 | 2.805 | 1.00 | 50.00 | C |
| ATOM | 2390 | NZ | LYS | B | 394 | −15.079 | 71.028 | 4.041 | 1.00 | 50.00 | N |
| ATOM | 2391 | N | ILE | B | 395 | −12.642 | 66.080 | −2.185 | 1.00 | 50.00 | N |
| ATOM | 2392 | CA | ILE | B | 395 | −11.576 | 65.644 | −3.082 | 1.00 | 50.00 | C |
| ATOM | 2393 | C | ILE | B | 395 | −10.278 | 66.376 | −2.746 | 1.00 | 50.00 | C |
| ATOM | 2394 | O | ILE | B | 395 | −10.299 | 67.574 | −2.467 | 1.00 | 50.00 | O |
| ATOM | 2395 | CB | ILE | B | 395 | −12.245 | 65.901 | −4.438 | 1.00 | 50.00 | C |
| ATOM | 2396 | CG1 | ILE | B | 395 | −13.435 | 65.003 | −4.790 | 1.00 | 50.00 | C |
| ATOM | 2397 | CG2 | ILE | B | 395 | −11.196 | 65.720 | −5.529 | 1.00 | 50.00 | C |
| ATOM | 2398 | CD1 | ILE | B | 395 | −13.851 | 64.752 | −6.239 | 1.00 | 50.00 | C |
| ATOM | 2399 | N | MET | B | 396 | −9.150 | 65.685 | −2.789 | 1.00 | 50.00 | N |
| ATOM | 2400 | CA | MET | B | 396 | −7.821 | 66.285 | −2.654 | 1.00 | 50.00 | C |
| ATOM | 2401 | C | MET | B | 396 | −6.735 | 65.579 | −3.456 | 1.00 | 50.00 | C |
| ATOM | 2402 | O | MET | B | 396 | −6.062 | 66.244 | −4.241 | 1.00 | 50.00 | O |
| ATOM | 2403 | CB | MET | B | 396 | −7.316 | 66.435 | −1.217 | 1.00 | 50.00 | C |
| ATOM | 2404 | CG | MET | B | 396 | −7.702 | 67.763 | −0.573 | 1.00 | 50.00 | C |
| ATOM | 2405 | SD | MET | B | 396 | −7.042 | 67.843 | 1.099 | 1.00 | 50.00 | S |
| ATOM | 2406 | CE | MET | B | 396 | −7.813 | 69.411 | 1.531 | 1.00 | 50.00 | C |
| ATOM | 2407 | N | THR | B | 397 | −6.456 | 64.319 | −3.173 | 1.00 | 50.00 | N |
| ATOM | 2408 | CA | THR | B | 397 | −5.330 | 63.556 | −3.711 | 1.00 | 50.00 | C |
| ATOM | 2409 | C | THR | B | 397 | −5.433 | 62.207 | −3.018 | 1.00 | 50.00 | C |
| ATOM | 2410 | O | THR | B | 397 | −5.714 | 62.096 | −1.824 | 1.00 | 50.00 | O |
| ATOM | 2411 | CB | THR | B | 397 | −3.905 | 64.103 | −3.595 | 1.00 | 50.00 | C |
| ATOM | 2412 | OG1 | THR | B | 397 | −3.017 | 63.291 | −4.351 | 1.00 | 50.00 | O |
| ATOM | 2413 | CG2 | THR | B | 397 | −3.310 | 64.273 | −2.199 | 1.00 | 50.00 | C |
| ATOM | 2414 | N | SER | B | 398 | −5.115 | 61.171 | −3.777 | 1.00 | 50.00 | N |
| ATOM | 2415 | CA | SER | B | 398 | −4.997 | 59.839 | −3.189 | 1.00 | 50.00 | C |
| ATOM | 2416 | C | SER | B | 398 | −3.678 | 59.865 | −2.431 | 1.00 | 50.00 | C |
| ATOM | 2417 | O | SER | B | 398 | −2.796 | 60.708 | −2.596 | 1.00 | 50.00 | O |
| ATOM | 2418 | CB | SER | B | 398 | −5.145 | 58.706 | −4.203 | 1.00 | 50.00 | C |
| ATOM | 2419 | OG | SER | B | 398 | −4.192 | 58.795 | −5.253 | 1.00 | 50.00 | O |
| ATOM | 2420 | N | LYS | B | 399 | −3.541 | 58.871 | −1.575 | 1.00 | 50.00 | N |
| ATOM | 2421 | CA | LYS | B | 399 | −2.256 | 58.632 | −0.918 | 1.00 | 50.00 | C |
| ATOM | 2422 | C | LYS | B | 399 | −1.177 | 58.107 | −1.862 | 1.00 | 50.00 | C |
| ATOM | 2423 | O | LYS | B | 399 | −0.361 | 57.317 | −1.393 | 1.00 | 50.00 | O |
| ATOM | 2424 | CB | LYS | B | 399 | −2.644 | 57.735 | 0.259 | 1.00 | 50.00 | C |
| ATOM | 2425 | CG | LYS | B | 399 | −3.376 | 58.510 | 1.353 | 1.00 | 50.00 | C |
| ATOM | 2426 | CD | LYS | B | 399 | −3.720 | 57.676 | 2.584 | 1.00 | 50.00 | C |
| ATOM | 2427 | CE | LYS | B | 399 | −4.451 | 58.580 | 3.573 | 1.00 | 50.00 | C |
| ATOM | 2428 | NZ | LYS | B | 399 | −4.692 | 57.868 | 4.836 | 1.00 | 50.00 | N |
| ATOM | 2429 | N | THR | B | 400 | −1.249 | 58.273 | −3.177 | 1.00 | 50.00 | N |
| ATOM | 2430 | CA | THR | B | 400 | −1.078 | 57.152 | −4.103 | 1.00 | 50.00 | C |
| ATOM | 2431 | C | THR | B | 400 | −0.579 | 57.562 | −5.486 | 1.00 | 50.00 | C |
| ATOM | 2432 | O | THR | B | 400 | −1.305 | 58.281 | −6.172 | 1.00 | 50.00 | O |
| ATOM | 2433 | CB | THR | B | 400 | −2.159 | 56.070 | −4.045 | 1.00 | 50.00 | C |
| ATOM | 2434 | OG1 | THR | B | 400 | −3.403 | 56.427 | −4.625 | 1.00 | 50.00 | O |
| ATOM | 2435 | CG2 | THR | B | 400 | −2.490 | 55.577 | −2.637 | 1.00 | 50.00 | C |
| ATOM | 2436 | N | ASP | B | 401 | 0.608 | 57.159 | −5.917 | 1.00 | 50.00 | N |
| ATOM | 2437 | CA | ASP | B | 401 | 1.266 | 57.673 | −7.122 | 1.00 | 50.00 | C |
| ATOM | 2438 | C | ASP | B | 401 | 0.548 | 57.226 | −8.390 | 1.00 | 50.00 | C |
| ATOM | 2439 | O | ASP | B | 401 | 0.079 | 58.079 | −9.142 | 1.00 | 50.00 | O |
| ATOM | 2440 | CB | ASP | B | 401 | 2.774 | 57.439 | −7.246 | 1.00 | 50.00 | C |
| ATOM | 2441 | CG | ASP | B | 401 | 3.473 | 58.253 | −8.326 | 1.00 | 50.00 | C |
| ATOM | 2442 | OD1 | ASP | B | 401 | 3.593 | 59.487 | −8.164 | 1.00 | 50.00 | O |
| ATOM | 2443 | OD2 | ASP | B | 401 | 3.904 | 57.677 | −9.348 | 1.00 | 50.00 | O |
| ATOM | 2444 | N | VAL | B | 402 | 0.507 | 55.926 | −8.631 | 1.00 | 50.00 | N |
| ATOM | 2445 | CA | VAL | B | 402 | −0.129 | 55.327 | −9.803 | 1.00 | 50.00 | C |
| ATOM | 2446 | C | VAL | B | 402 | −0.668 | 53.997 | −9.294 | 1.00 | 50.00 | C |
| ATOM | 2447 | O | VAL | B | 402 | 0.049 | 53.173 | −8.727 | 1.00 | 50.00 | O |
| ATOM | 2448 | CB | VAL | B | 402 | 0.797 | 55.126 | −11.006 | 1.00 | 50.00 | C |
| ATOM | 2449 | CG1 | VAL | B | 402 | 0.179 | 54.320 | −12.149 | 1.00 | 50.00 | C |
| ATOM | 2450 | CG2 | VAL | B | 402 | 1.298 | 56.444 | −11.595 | 1.00 | 50.00 | C |
| ATOM | 2451 | N | SER | B | 403 | −1.929 | 53.806 | −9.637 | 1.00 | 50.00 | N |
| ATOM | 2452 | CA | SER | B | 403 | −2.673 | 52.548 | −9.614 | 1.00 | 50.00 | C |
| ATOM | 2453 | C | SER | B | 403 | −4.094 | 53.045 | −9.849 | 1.00 | 50.00 | C |
| ATOM | 2454 | O | SER | B | 403 | −4.880 | 53.032 | −8.902 | 1.00 | 50.00 | O |
| ATOM | 2455 | CB | SER | B | 403 | −2.547 | 51.738 | −8.322 | 1.00 | 50.00 | C |
| ATOM | 2456 | OG | SER | B | 403 | −3.082 | 50.430 | −8.460 | 1.00 | 50.00 | O |
| ATOM | 2457 | N | SER | B | 404 | −4.467 | 53.530 | −11.025 | 1.00 | 50.00 | N |
| ATOM | 2458 | CA | SER | B | 404 | −5.812 | 54.065 | −11.247 | 1.00 | 50.00 | C |
| ATOM | 2459 | C | SER | B | 404 | −6.769 | 52.935 | −11.609 | 1.00 | 50.00 | C |
| ATOM | 2460 | O | SER | B | 404 | −7.211 | 52.776 | −12.746 | 1.00 | 50.00 | O |
| ATOM | 2461 | CB | SER | B | 404 | −5.782 | 55.208 | −12.260 | 1.00 | 50.00 | C |
| ATOM | 2462 | OG | SER | B | 404 | −5.081 | 56.339 | −11.763 | 1.00 | 50.00 | O |
| ATOM | 2463 | N | SER | B | 405 | −7.007 | 52.101 | −10.610 | 1.00 | 50.00 | N |
| ATOM | 2464 | CA | SER | B | 405 | −7.066 | 50.643 | −10.707 | 1.00 | 50.00 | C |
| ATOM | 2465 | C | SER | B | 405 | −6.690 | 50.019 | −9.368 | 1.00 | 50.00 | C |
| ATOM | 2466 | O | SER | B | 405 | −5.666 | 50.339 | −8.768 | 1.00 | 50.00 | O |
| ATOM | 2467 | CB | SER | B | 405 | −6.401 | 49.926 | −11.882 | 1.00 | 50.00 | C |
| ATOM | 2468 | OG | SER | B | 405 | −4.997 | 49.775 | −11.744 | 1.00 | 50.00 | O |
| ATOM | 2469 | N | VAL | B | 406 | −7.530 | 49.118 | −8.889 | 1.00 | 50.00 | N |
| ATOM | 2470 | CA | VAL | B | 406 | −7.406 | 48.428 | −7.604 | 1.00 | 50.00 | C |
| ATOM | 2471 | C | VAL | B | 406 | −8.025 | 47.054 | −7.829 | 1.00 | 50.00 | C |
| ATOM | 2472 | O | VAL | B | 406 | −9.044 | 46.907 | −8.504 | 1.00 | 50.00 | O |
| ATOM | 2473 | CB | VAL | B | 406 | −8.104 | 49.167 | −6.457 | 1.00 | 50.00 | C |
| ATOM | 2474 | CG1 | VAL | B | 406 | −8.290 | 48.379 | −5.160 | 1.00 | 50.00 | C |
| ATOM | 2475 | CG2 | VAL | B | 406 | −7.404 | 50.473 | −6.084 | 1.00 | 50.00 | C |
| ATOM | 2476 | N | ILE | B | 407 | −7.494 | 46.067 | −7.127 | 1.00 | 50.00 | N |
| ATOM | 2477 | CA | ILE | B | 407 | −8.162 | 44.778 | −6.950 | 1.00 | 50.00 | C |
| ATOM | 2478 | C | ILE | B | 407 | −9.156 | 44.711 | −5.795 | 1.00 | 50.00 | C |
| ATOM | 2479 | O | ILE | B | 407 | −8.941 | 45.277 | −4.724 | 1.00 | 50.00 | O |
| ATOM | 2480 | CB | ILE | B | 407 | −7.119 | 43.658 | −6.964 | 1.00 | 50.00 | C |
| ATOM | 2481 | CG1 | ILE | B | 407 | −7.670 | 42.461 | −7.735 | 1.00 | 50.00 | C |
| ATOM | 2482 | CG2 | ILE | B | 407 | −6.615 | 43.261 | −5.578 | 1.00 | 50.00 | C |
| ATOM | 2483 | CD1 | ILE | B | 407 | −7.685 | 42.714 | −9.242 | 1.00 | 50.00 | C |
| ATOM | 2484 | N | THR | B | 408 | −10.187 | 43.906 | −5.986 | 1.00 | 50.00 | N |
| ATOM | 2485 | CA | THR | B | 408 | −11.226 | 43.617 | −4.997 | 1.00 | 50.00 | C |
| ATOM | 2486 | C | THR | B | 408 | −11.881 | 42.312 | −5.431 | 1.00 | 50.00 | C |
| ATOM | 2487 | O | THR | B | 408 | −12.440 | 42.270 | −6.527 | 1.00 | 50.00 | O |
| ATOM | 2488 | CB | THR | B | 408 | −12.267 | 44.737 | −4.957 | 1.00 | 50.00 | C |
| ATOM | 2489 | OG1 | THR | B | 408 | −11.682 | 45.918 | −4.428 | 1.00 | 50.00 | O |
| ATOM | 2490 | CG2 | THR | B | 408 | −13.540 | 44.453 | −4.162 | 1.00 | 50.00 | C |
| ATOM | 2491 | N | SER | B | 409 | −11.927 | 41.331 | −4.543 | 1.00 | 50.00 | N |
| ATOM | 2492 | CA | SER | B | 409 | −12.766 | 40.135 | −4.649 | 1.00 | 50.00 | C |
| ATOM | 2493 | C | SER | B | 409 | −12.836 | 39.543 | −6.050 | 1.00 | 50.00 | C |
| ATOM | 2494 | O | SER | B | 409 | −13.831 | 39.767 | −6.739 | 1.00 | 50.00 | O |
| ATOM | 2495 | CB | SER | B | 409 | −14.190 | 40.364 | −4.140 | 1.00 | 50.00 | C |
| ATOM | 2496 | OG | SER | B | 409 | −14.246 | 40.663 | −2.753 | 1.00 | 50.00 | O |
| ATOM | 2497 | N | LEU | B | 410 | −11.771 | 38.935 | −6.544 | 1.00 | 50.00 | N |
| ATOM | 2498 | CA | LEU | B | 410 | −11.731 | 38.460 | −7.927 | 1.00 | 50.00 | C |
| ATOM | 2499 | C | LEU | B | 410 | −11.809 | 39.485 | −9.056 | 1.00 | 50.00 | C |
| ATOM | 2500 | O | LEU | B | 410 | −12.321 | 39.182 | −10.132 | 1.00 | 50.00 | O |
| ATOM | 2501 | CB | LEU | B | 410 | −12.605 | 37.211 | −8.060 | 1.00 | 50.00 | C |
| ATOM | 2502 | CG | LEU | B | 410 | −14.127 | 37.198 | −8.214 | 1.00 | 50.00 | C |
| ATOM | 2503 | CD1 | LEU | B | 410 | −14.823 | 38.221 | −9.113 | 1.00 | 50.00 | C |
| ATOM | 2504 | CD2 | LEU | B | 410 | −14.508 | 35.826 | −8.765 | 1.00 | 50.00 | C |
| ATOM | 2505 | N | GLY | B | 411 | −11.380 | 40.730 | −8.934 | 1.00 | 50.00 | N |
| ATOM | 2506 | CA | GLY | B | 411 | −11.866 | 41.764 | −9.847 | 1.00 | 50.00 | C |
| ATOM | 2507 | C | GLY | B | 411 | −11.120 | 43.086 | −9.794 | 1.00 | 50.00 | C |
| ATOM | 2508 | O | GLY | B | 411 | −10.611 | 43.456 | −8.738 | 1.00 | 50.00 | O |
| ATOM | 2509 | N | ALA | B | 412 | −11.086 | 43.794 | −10.910 | 1.00 | 50.00 | N |
| ATOM | 2510 | CA | ALA | B | 412 | −10.262 | 44.994 | −11.045 | 1.00 | 50.00 | C |
| ATOM | 2511 | C | ALA | B | 412 | −11.148 | 46.208 | −11.291 | 1.00 | 50.00 | C |
| ATOM | 2512 | O | ALA | B | 412 | −11.487 | 46.491 | −12.438 | 1.00 | 50.00 | O |
| ATOM | 2513 | CB | ALA | B | 412 | −9.214 | 44.743 | −12.129 | 1.00 | 50.00 | C |
| ATOM | 2514 | N | ILE | B | 413 | −11.489 | 46.951 | −10.250 | 1.00 | 50.00 | N |
| ATOM | 2515 | CA | ILE | B | 413 | −12.125 | 48.267 | −10.344 | 1.00 | 50.00 | C |
| ATOM | 2516 | C | ILE | B | 413 | −11.091 | 49.268 | −10.844 | 1.00 | 50.00 | C |
| ATOM | 2517 | O | ILE | B | 413 | −10.055 | 49.407 | −10.196 | 1.00 | 50.00 | O |
| ATOM | 2518 | CB | ILE | B | 413 | −12.724 | 48.673 | −8.994 | 1.00 | 50.00 | C |
| ATOM | 2519 | CG1 | ILE | B | 413 | −14.007 | 47.900 | −8.679 | 1.00 | 50.00 | C |
| ATOM | 2520 | CG2 | ILE | B | 413 | −12.985 | 50.164 | −8.780 | 1.00 | 50.00 | C |
| ATOM | 2521 | CD1 | ILE | B | 413 | −14.006 | 47.271 | −7.288 | 1.00 | 50.00 | C |
| ATOM | 2522 | N | VAL | B | 414 | −11.363 | 49.954 | −11.941 | 1.00 | 50.00 | N |
| ATOM | 2523 | CA | VAL | B | 414 | −10.365 | 50.767 | −12.637 | 1.00 | 50.00 | C |
| ATOM | 2524 | C | VAL | B | 414 | −10.991 | 52.078 | −13.103 | 1.00 | 50.00 | C |
| ATOM | 2525 | O | VAL | B | 414 | −12.097 | 52.068 | −13.644 | 1.00 | 50.00 | O |
| ATOM | 2526 | CB | VAL | B | 414 | −9.569 | 49.978 | −13.681 | 1.00 | 50.00 | C |
| ATOM | 2527 | CG1 | VAL | B | 414 | −9.171 | 48.530 | −13.394 | 1.00 | 50.00 | C |
| ATOM | 2528 | CG2 | VAL | B | 414 | −10.223 | 49.913 | −15.059 | 1.00 | 50.00 | C |
| ATOM | 2529 | N | SER | B | 415 | −10.277 | 53.188 | −13.004 | 1.00 | 50.00 | N |
| ATOM | 2530 | CA | SER | B | 415 | −10.632 | 54.437 | −13.679 | 1.00 | 50.00 | C |
| ATOM | 2531 | C | SER | B | 415 | −10.236 | 54.371 | −15.150 | 1.00 | 50.00 | C |
| ATOM | 2532 | O | SER | B | 415 | −9.154 | 54.810 | −15.540 | 1.00 | 50.00 | O |
| ATOM | 2533 | CB | SER | B | 415 | −9.996 | 55.634 | −12.971 | 1.00 | 50.00 | C |
| ATOM | 2534 | OG | SER | B | 415 | −10.441 | 55.767 | −11.629 | 1.00 | 50.00 | O |
| ATOM | 2535 | N | CYS | B | 416 | −11.104 | 53.795 | −15.967 | 1.00 | 50.00 | N |
| ATOM | 2536 | CA | CYS | B | 416 | −10.839 | 53.516 | −17.379 | 1.00 | 50.00 | C |
| ATOM | 2537 | C | CYS | B | 416 | −10.623 | 54.742 | −18.255 | 1.00 | 50.00 | C |
| ATOM | 2538 | O | CYS | B | 416 | −9.532 | 54.923 | −18.795 | 1.00 | 50.00 | O |
| ATOM | 2539 | CB | CYS | B | 416 | −11.915 | 52.609 | −17.974 | 1.00 | 50.00 | C |
| ATOM | 2540 | SG | CYS | B | 416 | −11.625 | 50.905 | −17.480 | 1.00 | 50.00 | S |
| ATOM | 2541 | N | TYR | B | 417 | −11.637 | 55.583 | −18.368 | 1.00 | 50.00 | N |
| ATOM | 2542 | CA | TYR | B | 417 | −11.526 | 56.903 | −18.986 | 1.00 | 50.00 | C |
| ATOM | 2543 | C | TYR | B | 417 | −10.686 | 57.921 | −18.221 | 1.00 | 50.00 | C |
| ATOM | 2544 | O | TYR | B | 417 | −9.973 | 58.677 | −18.881 | 1.00 | 50.00 | O |
| ATOM | 2545 | CB | TYR | B | 417 | −12.963 | 57.351 | −19.255 | 1.00 | 50.00 | C |
| ATOM | 2546 | CG | TYR | B | 417 | −13.094 | 58.625 | −20.060 | 1.00 | 50.00 | C |
| ATOM | 2547 | CD1 | TYR | B | 417 | −12.904 | 58.600 | −21.396 | 1.00 | 50.00 | C |
| ATOM | 2548 | CD2 | TYR | B | 417 | −13.262 | 59.806 | −19.431 | 1.00 | 50.00 | C |
| ATOM | 2549 | CE1 | TYR | B | 417 | −12.851 | 59.752 | −22.094 | 1.00 | 50.00 | C |
| ATOM | 2550 | CE2 | TYR | B | 417 | −13.200 | 60.960 | −20.125 | 1.00 | 50.00 | C |
| ATOM | 2551 | CZ | TYR | B | 417 | −12.988 | 60.932 | −21.457 | 1.00 | 50.00 | C |
| ATOM | 2552 | OH | TYR | B | 417 | −12.837 | 62.096 | −22.163 | 1.00 | 50.00 | O |
| ATOM | 2553 | N | GLY | B | 418 | −10.748 | 57.969 | −16.900 | 1.00 | 50.00 | N |
| ATOM | 2554 | CA | GLY | B | 418 | −9.930 | 58.852 | −16.068 | 1.00 | 50.00 | C |
| ATOM | 2555 | C | GLY | B | 418 | −8.429 | 58.827 | −16.323 | 1.00 | 50.00 | C |
| ATOM | 2556 | O | GLY | B | 418 | −7.885 | 59.809 | −16.827 | 1.00 | 50.00 | O |
| ATOM | 2557 | N | LYS | B | 419 | −7.797 | 57.692 | −16.076 | 1.00 | 50.00 | N |
| ATOM | 2558 | CA | LYS | B | 419 | −6.438 | 57.414 | −16.541 | 1.00 | 50.00 | C |
| ATOM | 2559 | C | LYS | B | 419 | −6.247 | 57.337 | −18.053 | 1.00 | 50.00 | C |
| ATOM | 2560 | O | LYS | B | 419 | −5.372 | 58.062 | −18.523 | 1.00 | 50.00 | O |
| ATOM | 2561 | CB | LYS | B | 419 | −5.923 | 56.142 | −15.867 | 1.00 | 50.00 | C |
| ATOM | 2562 | CG | LYS | B | 419 | −4.423 | 55.885 | −16.008 | 1.00 | 50.00 | C |
| ATOM | 2563 | CD | LYS | B | 419 | −3.611 | 56.622 | −14.945 | 1.00 | 50.00 | C |
| ATOM | 2564 | CE | LYS | B | 419 | −2.097 | 56.476 | −15.069 | 1.00 | 50.00 | C |
| ATOM | 2565 | NZ | LYS | B | 419 | −1.473 | 57.143 | −13.917 | 1.00 | 50.00 | N |
| ATOM | 2566 | N | THR | B | 420 | −6.962 | 56.463 | −18.746 | 1.00 | 50.00 | N |
| ATOM | 2567 | CA | THR | B | 420 | −6.680 | 55.889 | −20.066 | 1.00 | 50.00 | C |
| ATOM | 2568 | C | THR | B | 420 | −6.595 | 54.363 | −20.079 | 1.00 | 50.00 | C |
| ATOM | 2569 | O | THR | B | 420 | −6.483 | 53.795 | −21.165 | 1.00 | 50.00 | O |
| ATOM | 2570 | CB | THR | B | 420 | −5.636 | 56.596 | −20.938 | 1.00 | 50.00 | C |
| ATOM | 2571 | OG1 | THR | B | 420 | −6.003 | 57.960 | −21.088 | 1.00 | 50.00 | O |
| ATOM | 2572 | CG2 | THR | B | 420 | −5.373 | 56.131 | −22.369 | 1.00 | 50.00 | C |
| ATOM | 2573 | N | LYS | B | 421 | −6.707 | 53.630 | −18.981 | 1.00 | 50.00 | N |
| ATOM | 2574 | CA | LYS | B | 421 | −6.352 | 52.218 | −18.826 | 1.00 | 50.00 | C |
| ATOM | 2575 | C | LYS | B | 421 | −6.604 | 51.212 | −19.945 | 1.00 | 50.00 | C |
| ATOM | 2576 | O | LYS | B | 421 | −5.649 | 50.623 | −20.449 | 1.00 | 50.00 | O |
| ATOM | 2577 | CB | LYS | B | 421 | −6.902 | 51.714 | −17.489 | 1.00 | 50.00 | C |
| ATOM | 2578 | CG | LYS | B | 421 | −6.289 | 52.213 | −16.181 | 1.00 | 50.00 | C |
| ATOM | 2579 | CD | LYS | B | 421 | −4.904 | 51.658 | −15.861 | 1.00 | 50.00 | C |
| ATOM | 2580 | CE | LYS | B | 421 | −4.339 | 52.235 | −14.564 | 1.00 | 50.00 | C |
| ATOM | 2581 | NZ | LYS | B | 421 | −2.892 | 51.996 | −14.463 | 1.00 | 50.00 | N |
| ATOM | 2582 | N | CYS | B | 422 | −7.836 | 50.875 | −20.290 | 1.00 | 50.00 | N |
| ATOM | 2583 | CA | CYS | B | 422 | −8.268 | 49.489 | −20.111 | 1.00 | 50.00 | C |
| ATOM | 2584 | C | CYS | B | 422 | −8.434 | 48.701 | −21.405 | 1.00 | 50.00 | C |
| ATOM | 2585 | O | CYS | B | 422 | −9.511 | 48.663 | −21.999 | 1.00 | 50.00 | O |
| ATOM | 2586 | CB | CYS | B | 422 | −9.597 | 49.526 | −19.363 | 1.00 | 50.00 | C |
| ATOM | 2587 | SG | CYS | B | 422 | −9.702 | 50.504 | −17.860 | 1.00 | 50.00 | S |
| ATOM | 2588 | N | THR | B | 423 | −7.398 | 47.996 | −21.826 | 1.00 | 50.00 | N |
| ATOM | 2589 | CA | THR | B | 423 | −7.459 | 47.068 | −22.956 | 1.00 | 50.00 | C |
| ATOM | 2590 | C | THR | B | 423 | −7.933 | 45.704 | −22.466 | 1.00 | 50.00 | C |
| ATOM | 2591 | O | THR | B | 423 | −7.280 | 45.086 | −21.626 | 1.00 | 50.00 | O |
| ATOM | 2592 | CB | THR | B | 423 | −6.085 | 47.033 | −23.627 | 1.00 | 50.00 | C |
| ATOM | 2593 | OG1 | THR | B | 423 | −5.813 | 48.299 | −24.210 | 1.00 | 50.00 | O |
| ATOM | 2594 | CG2 | THR | B | 423 | −5.898 | 45.983 | −24.722 | 1.00 | 50.00 | C |
| ATOM | 2595 | N | ALA | B | 424 | −9.012 | 45.192 | −23.034 | 1.00 | 50.00 | N |
| ATOM | 2596 | CA | ALA | B | 424 | −9.403 | 43.791 | −22.877 | 1.00 | 50.00 | C |
| ATOM | 2597 | C | ALA | B | 424 | −8.828 | 42.969 | −24.025 | 1.00 | 50.00 | C |
| ATOM | 2598 | O | ALA | B | 424 | −9.302 | 43.053 | −25.158 | 1.00 | 50.00 | O |
| ATOM | 2599 | CB | ALA | B | 424 | −10.925 | 43.719 | −22.783 | 1.00 | 50.00 | C |
| ATOM | 2600 | N | SER | B | 425 | −7.815 | 42.169 | −23.735 | 1.00 | 50.00 | N |
| ATOM | 2601 | CA | SER | B | 425 | −7.230 | 41.211 | −24.677 | 1.00 | 50.00 | C |
| ATOM | 2602 | C | SER | B | 425 | −8.082 | 40.036 | −25.149 | 1.00 | 50.00 | C |
| ATOM | 2603 | O | SER | B | 425 | −7.625 | 39.322 | −26.040 | 1.00 | 50.00 | O |
| ATOM | 2604 | CB | SER | B | 425 | −5.877 | 40.751 | −24.132 | 1.00 | 50.00 | C |
| ATOM | 2605 | OG | SER | B | 425 | −4.904 | 41.786 | −24.174 | 1.00 | 50.00 | O |
| ATOM | 2606 | N | ASN | B | 426 | −9.289 | 39.811 | −24.651 | 1.00 | 50.00 | N |
| ATOM | 2607 | CA | ASN | B | 426 | −10.197 | 38.765 | −25.125 | 1.00 | 50.00 | C |
| ATOM | 2608 | C | ASN | B | 426 | −10.743 | 39.092 | −26.511 | 1.00 | 50.00 | C |
| ATOM | 2609 | O | ASN | B | 426 | −10.692 | 40.216 | −27.009 | 1.00 | 50.00 | O |
| ATOM | 2610 | CB | ASN | B | 426 | −11.288 | 38.517 | −24.080 | 1.00 | 50.00 | C |
| ATOM | 2611 | CG | ASN | B | 426 | −12.144 | 37.271 | −24.274 | 1.00 | 50.00 | C |
| ATOM | 2612 | OD1 | ASN | B | 426 | −11.721 | 36.150 | −23.996 | 1.00 | 50.00 | O |
| ATOM | 2613 | ND2 | ASN | B | 426 | −13.354 | 37.401 | −24.787 | 1.00 | 50.00 | N |
| ATOM | 2614 | N | LYS | B | 427 | −11.281 | 38.063 | −27.143 | 1.00 | 50.00 | N |
| ATOM | 2615 | CA | LYS | B | 427 | −12.002 | 38.114 | −28.414 | 1.00 | 50.00 | C |
| ATOM | 2616 | C | LYS | B | 427 | −13.034 | 39.209 | −28.664 | 1.00 | 50.00 | C |
| ATOM | 2617 | O | LYS | B | 427 | −12.983 | 39.781 | −29.752 | 1.00 | 50.00 | O |
| ATOM | 2618 | CB | LYS | B | 427 | −12.627 | 36.738 | −28.643 | 1.00 | 50.00 | C |
| ATOM | 2619 | CG | LYS | B | 427 | −11.627 | 35.597 | −28.829 | 1.00 | 50.00 | C |
| ATOM | 2620 | CD | LYS | B | 427 | −12.303 | 34.276 | −29.187 | 1.00 | 50.00 | C |
| ATOM | 2621 | CE | LYS | B | 427 | −11.287 | 33.149 | −29.351 | 1.00 | 50.00 | C |
| ATOM | 2622 | NZ | LYS | B | 427 | −11.960 | 31.905 | −29.751 | 1.00 | 50.00 | N |
| ATOM | 2623 | N | ASN | B | 428 | −13.946 | 39.515 | −27.754 | 1.00 | 50.00 | N |
| ATOM | 2624 | CA | ASN | B | 428 | −15.037 | 40.466 | −27.984 | 1.00 | 50.00 | C |
| ATOM | 2625 | C | ASN | B | 428 | −14.697 | 41.946 | −28.157 | 1.00 | 50.00 | C |
| ATOM | 2626 | O | ASN | B | 428 | −15.600 | 42.780 | −28.108 | 1.00 | 50.00 | O |
| ATOM | 2627 | CB | ASN | B | 428 | −16.084 | 40.244 | −26.888 | 1.00 | 50.00 | C |
| ATOM | 2628 | CG | ASN | B | 428 | −16.811 | 38.910 | −26.775 | 1.00 | 50.00 | C |
| ATOM | 2629 | OD1 | ASN | B | 428 | −16.711 | 38.214 | −25.765 | 1.00 | 50.00 | O |
| ATOM | 2630 | ND2 | ASN | B | 428 | −17.595 | 38.526 | −27.765 | 1.00 | 50.00 | N |
| ATOM | 2631 | N | ARG | B | 429 | −13.453 | 42.356 | −28.355 | 1.00 | 50.00 | N |
| ATOM | 2632 | CA | ARG | B | 429 | −12.843 | 43.486 | −27.650 | 1.00 | 50.00 | C |
| ATOM | 2633 | C | ARG | B | 429 | −11.602 | 44.024 | −28.359 | 1.00 | 50.00 | C |
| ATOM | 2634 | O | ARG | B | 429 | −10.902 | 43.250 | −29.012 | 1.00 | 50.00 | O |
| ATOM | 2635 | CB | ARG | B | 429 | −12.406 | 43.046 | −26.251 | 1.00 | 50.00 | C |
| ATOM | 2636 | CG | ARG | B | 429 | −13.449 | 42.537 | −25.255 | 1.00 | 50.00 | C |
| ATOM | 2637 | CD | ARG | B | 429 | −14.655 | 43.441 | −25.009 | 1.00 | 50.00 | C |
| ATOM | 2638 | NE | ARG | B | 429 | −14.127 | 44.686 | −24.459 | 1.00 | 50.00 | N |
| ATOM | 2639 | CZ | ARG | B | 429 | −14.850 | 45.711 | −24.050 | 1.00 | 50.00 | C |
| ATOM | 2640 | NH1 | ARG | B | 429 | −16.168 | 45.780 | −24.004 | 1.00 | 50.00 | N |
| ATOM | 2641 | NH2 | ARG | B | 429 | −14.128 | 46.735 | −23.646 | 1.00 | 50.00 | N |
| ATOM | 2642 | N | GLY | B | 430 | −11.110 | 45.251 | −28.280 | 1.00 | 50.00 | N |
| ATOM | 2643 | CA | GLY | B | 430 | −11.752 | 46.424 | −27.686 | 1.00 | 50.00 | C |
| ATOM | 2644 | C | GLY | B | 430 | −11.090 | 47.029 | −26.453 | 1.00 | 50.00 | C |
| ATOM | 2645 | O | GLY | B | 430 | −10.545 | 46.308 | −25.618 | 1.00 | 50.00 | O |
| ATOM | 2646 | N | ILE | B | 431 | −11.206 | 48.340 | −26.306 | 1.00 | 50.00 | N |
| ATOM | 2647 | CA | ILE | B | 431 | −10.898 | 49.083 | −25.081 | 1.00 | 50.00 | C |
| ATOM | 2648 | C | ILE | B | 431 | −12.197 | 49.242 | −24.296 | 1.00 | 50.00 | C |
| ATOM | 2649 | O | ILE | B | 431 | −13.217 | 49.652 | −24.850 | 1.00 | 50.00 | O |
| ATOM | 2650 | CB | ILE | B | 431 | −10.204 | 50.428 | −25.329 | 1.00 | 50.00 | C |
| ATOM | 2651 | CG1 | ILE | B | 431 | −8.851 | 50.294 | −26.031 | 1.00 | 50.00 | C |
| ATOM | 2652 | CG2 | ILE | B | 431 | −10.024 | 51.283 | −24.073 | 1.00 | 50.00 | C |
| ATOM | 2653 | CD1 | ILE | B | 431 | −8.117 | 51.576 | −26.423 | 1.00 | 50.00 | C |
| ATOM | 2654 | N | ILE | B | 432 | −12.151 | 49.008 | −22.993 | 1.00 | 50.00 | N |
| ATOM | 2655 | CA | ILE | B | 432 | −13.257 | 49.272 | −22.073 | 1.00 | 50.00 | C |
| ATOM | 2656 | C | ILE | B | 432 | −13.212 | 50.731 | −21.635 | 1.00 | 50.00 | C |
| ATOM | 2657 | O | ILE | B | 432 | −12.365 | 51.125 | −20.835 | 1.00 | 50.00 | O |
| ATOM | 2658 | CB | ILE | B | 432 | −13.399 | 48.239 | −20.951 | 1.00 | 50.00 | C |
| ATOM | 2659 | CG1 | ILE | B | 432 | −14.787 | 48.251 | −20.312 | 1.00 | 50.00 | C |
| ATOM | 2660 | CG2 | ILE | B | 432 | −12.335 | 48.174 | −19.858 | 1.00 | 50.00 | C |
| ATOM | 2661 | CD1 | ILE | B | 432 | −15.646 | 47.045 | −20.682 | 1.00 | 50.00 | C |
| ATOM | 2662 | N | LYS | B | 433 | −14.110 | 51.525 | −22.193 | 1.00 | 50.00 | N |
| ATOM | 2663 | CA | LYS | B | 433 | −14.212 | 52.959 | −21.922 | 1.00 | 50.00 | C |
| ATOM | 2664 | C | LYS | B | 433 | −15.424 | 53.243 | −21.042 | 1.00 | 50.00 | C |
| ATOM | 2665 | O | LYS | B | 433 | −16.440 | 52.549 | −21.075 | 1.00 | 50.00 | O |
| ATOM | 2666 | CB | LYS | B | 433 | −14.268 | 53.736 | −23.237 | 1.00 | 50.00 | C |
| ATOM | 2667 | CG | LYS | B | 433 | −12.979 | 53.686 | −24.057 | 1.00 | 50.00 | C |
| ATOM | 2668 | CD | LYS | B | 433 | −13.016 | 54.573 | −25.300 | 1.00 | 50.00 | C |
| ATOM | 2669 | CE | LYS | B | 433 | −11.747 | 54.439 | −26.139 | 1.00 | 50.00 | C |
| ATOM | 2670 | NZ | LYS | B | 433 | −11.799 | 55.336 | −27.302 | 1.00 | 50.00 | N |
| ATOM | 2671 | N | THR | B | 434 | −15.299 | 54.299 | −20.257 | 1.00 | 50.00 | N |
| ATOM | 2672 | CA | THR | B | 434 | −16.331 | 54.765 | −19.330 | 1.00 | 50.00 | C |
| ATOM | 2673 | C | THR | B | 434 | −16.718 | 56.179 | −19.747 | 1.00 | 50.00 | C |
| ATOM | 2674 | O | THR | B | 434 | −15.891 | 57.089 | −19.760 | 1.00 | 50.00 | O |
| ATOM | 2675 | CB | THR | B | 434 | −15.795 | 54.651 | −17.902 | 1.00 | 50.00 | C |
| ATOM | 2676 | OG1 | THR | B | 434 | −15.619 | 53.275 | −17.600 | 1.00 | 50.00 | O |
| ATOM | 2677 | CG2 | THR | B | 434 | −16.679 | 55.213 | −16.791 | 1.00 | 50.00 | C |
| ATOM | 2678 | N | PHE | B | 435 | −17.996 | 56.401 | −20.008 | 1.00 | 50.00 | N |
| ATOM | 2679 | CA | PHE | B | 435 | −18.564 | 57.744 | −20.132 | 1.00 | 50.00 | C |
| ATOM | 2680 | C | PHE | B | 435 | −18.666 | 58.454 | −18.784 | 1.00 | 50.00 | C |
| ATOM | 2681 | O | PHE | B | 435 | −18.558 | 57.849 | −17.718 | 1.00 | 50.00 | O |
| ATOM | 2682 | CB | PHE | B | 435 | −19.870 | 57.611 | −20.920 | 1.00 | 50.00 | C |
| ATOM | 2683 | CG | PHE | B | 435 | −20.515 | 58.907 | −21.366 | 1.00 | 50.00 | C |
| ATOM | 2684 | CD1 | PHE | B | 435 | −19.924 | 59.669 | −22.309 | 1.00 | 50.00 | C |
| ATOM | 2685 | CD2 | PHE | B | 435 | −21.569 | 59.400 | −20.682 | 1.00 | 50.00 | C |
| ATOM | 2686 | CE1 | PHE | B | 435 | −20.338 | 60.935 | −22.522 | 1.00 | 50.00 | C |
| ATOM | 2687 | CE2 | PHE | B | 435 | −21.982 | 60.668 | −20.885 | 1.00 | 50.00 | C |
| ATOM | 2688 | CZ | PHE | B | 435 | −21.360 | 61.438 | −21.801 | 1.00 | 50.00 | C |
| ATOM | 2689 | N | SER | B | 436 | −18.900 | 59.757 | −18.808 | 1.00 | 50.00 | N |
| ATOM | 2690 | CA | SER | B | 436 | −19.273 | 60.561 | −17.643 | 1.00 | 50.00 | C |
| ATOM | 2691 | C | SER | B | 436 | −20.462 | 60.026 | −16.852 | 1.00 | 50.00 | C |
| ATOM | 2692 | O | SER | B | 436 | −21.379 | 59.438 | −17.426 | 1.00 | 50.00 | O |
| ATOM | 2693 | CB | SER | B | 436 | −19.589 | 61.980 | −18.115 | 1.00 | 50.00 | C |
| ATOM | 2694 | OG | SER | B | 436 | −18.455 | 62.616 | −18.687 | 1.00 | 50.00 | O |
| ATOM | 2695 | N | ASN | B | 437 | −20.427 | 60.206 | −15.541 | 1.00 | 50.00 | N |
| ATOM | 2696 | CA | ASN | B | 437 | −21.350 | 59.596 | −14.581 | 1.00 | 50.00 | C |
| ATOM | 2697 | C | ASN | B | 437 | −20.877 | 58.285 | −13.963 | 1.00 | 50.00 | C |
| ATOM | 2698 | O | ASN | B | 437 | −21.129 | 58.081 | −12.776 | 1.00 | 50.00 | O |
| ATOM | 2699 | CB | ASN | B | 437 | −22.849 | 59.566 | −14.887 | 1.00 | 50.00 | C |
| ATOM | 2700 | CG | ASN | B | 437 | −23.490 | 60.891 | −15.276 | 1.00 | 50.00 | C |
| ATOM | 2701 | OD1 | ASN | B | 437 | −23.974 | 61.061 | −16.394 | 1.00 | 50.00 | O |
| ATOM | 2702 | ND2 | ASN | B | 437 | −23.495 | 61.865 | −14.385 | 1.00 | 50.00 | N |
| ATOM | 2703 | N | GLY | B | 438 | −20.182 | 57.419 | −14.684 | 1.00 | 50.00 | N |
| ATOM | 2704 | CA | GLY | B | 438 | −19.469 | 56.282 | −14.104 | 1.00 | 50.00 | C |
| ATOM | 2705 | C | GLY | B | 438 | −18.493 | 56.526 | −12.958 | 1.00 | 50.00 | C |
| ATOM | 2706 | O | GLY | B | 438 | −18.610 | 55.785 | −11.983 | 1.00 | 50.00 | O |
| ATOM | 2707 | N | CYS | B | 439 | −17.542 | 57.444 | −13.056 | 1.00 | 50.00 | N |
| ATOM | 2708 | CA | CYS | B | 439 | −16.324 | 57.499 | −12.240 | 1.00 | 50.00 | C |
| ATOM | 2709 | C | CYS | B | 439 | −15.311 | 56.424 | −12.624 | 1.00 | 50.00 | C |
| ATOM | 2710 | O | CYS | B | 439 | −14.129 | 56.714 | −12.803 | 1.00 | 50.00 | O |
| ATOM | 2711 | CB | CYS | B | 439 | −16.380 | 57.507 | −10.708 | 1.00 | 50.00 | C |
| ATOM | 2712 | SG | CYS | B | 439 | −16.520 | 58.998 | −9.706 | 1.00 | 50.00 | S |
| ATOM | 2713 | N | ASP | B | 440 | −15.743 | 55.173 | −12.585 | 1.00 | 50.00 | N |
| ATOM | 2714 | CA | ASP | B | 440 | −14.930 | 53.991 | −12.305 | 1.00 | 50.00 | C |
| ATOM | 2715 | C | ASP | B | 440 | −15.709 | 52.816 | −12.883 | 1.00 | 50.00 | C |
| ATOM | 2716 | O | ASP | B | 440 | −16.880 | 52.592 | −12.578 | 1.00 | 50.00 | O |
| ATOM | 2717 | CB | ASP | B | 440 | −14.734 | 53.742 | −10.809 | 1.00 | 50.00 | C |
| ATOM | 2718 | CG | ASP | B | 440 | −13.776 | 54.672 | −10.080 | 1.00 | 50.00 | C |
| ATOM | 2719 | OD1 | ASP | B | 440 | −12.589 | 54.731 | −10.468 | 1.00 | 50.00 | O |
| ATOM | 2720 | OD2 | ASP | B | 440 | −14.187 | 55.334 | −9.103 | 1.00 | 50.00 | O |
| ATOM | 2721 | N | TYR | B | 441 | −15.015 | 52.027 | −13.685 | 1.00 | 50.00 | N |
| ATOM | 2722 | CA | TYR | B | 441 | −15.532 | 50.787 | −14.264 | 1.00 | 50.00 | C |
| ATOM | 2723 | C | TYR | B | 441 | −15.318 | 49.576 | −13.360 | 1.00 | 50.00 | C |
| ATOM | 2724 | O | TYR | B | 441 | −14.404 | 49.552 | −12.536 | 1.00 | 50.00 | O |
| ATOM | 2725 | CB | TYR | B | 441 | −14.860 | 50.672 | −15.633 | 1.00 | 50.00 | C |
| ATOM | 2726 | CG | TYR | B | 441 | −15.509 | 49.646 | −16.537 | 1.00 | 50.00 | C |
| ATOM | 2727 | CD1 | TYR | B | 441 | −16.765 | 49.819 | −16.998 | 1.00 | 50.00 | C |
| ATOM | 2728 | CD2 | TYR | B | 441 | −14.923 | 48.435 | −16.631 | 1.00 | 50.00 | C |
| ATOM | 2729 | CE1 | TYR | B | 441 | −17.468 | 48.767 | −17.463 | 1.00 | 50.00 | C |
| ATOM | 2730 | CE2 | TYR | B | 441 | −15.621 | 47.383 | −17.102 | 1.00 | 50.00 | C |
| ATOM | 2731 | CZ | TYR | B | 441 | −16.905 | 47.543 | −17.480 | 1.00 | 50.00 | C |
| ATOM | 2732 | OH | TYR | B | 441 | −17.660 | 46.472 | −17.877 | 1.00 | 50.00 | O |
| ATOM | 2733 | N | VAL | B | 442 | −16.134 | 48.549 | −13.534 | 1.00 | 50.00 | N |
| ATOM | 2734 | CA | VAL | B | 442 | −16.044 | 47.281 | −12.807 | 1.00 | 50.00 | C |
| ATOM | 2735 | C | VAL | B | 442 | −14.926 | 46.377 | −13.317 | 1.00 | 50.00 | C |
| ATOM | 2736 | O | VAL | B | 442 | −14.025 | 46.095 | −12.529 | 1.00 | 50.00 | O |
| ATOM | 2737 | CB | VAL | B | 442 | −17.420 | 46.622 | −12.663 | 1.00 | 50.00 | C |
| ATOM | 2738 | CG1 | VAL | B | 442 | −17.398 | 45.184 | −12.145 | 1.00 | 50.00 | C |
| ATOM | 2739 | CG2 | VAL | B | 442 | −18.354 | 47.402 | −11.738 | 1.00 | 50.00 | C |
| ATOM | 2740 | N | SER | B | 443 | −14.992 | 45.831 | −14.521 | 1.00 | 50.00 | N |
| ATOM | 2741 | CA | SER | B | 443 | −14.044 | 44.870 | −15.088 | 1.00 | 50.00 | C |
| ATOM | 2742 | C | SER | B | 443 | −13.949 | 43.567 | −14.303 | 1.00 | 50.00 | C |
| ATOM | 2743 | O | SER | B | 443 | −13.035 | 43.391 | −13.498 | 1.00 | 50.00 | O |
| ATOM | 2744 | CB | SER | B | 443 | −12.658 | 45.403 | −15.455 | 1.00 | 50.00 | C |
| ATOM | 2745 | OG | SER | B | 443 | −12.658 | 46.265 | −16.583 | 1.00 | 50.00 | O |
| ATOM | 2746 | N | ASN | B | 444 | −14.892 | 42.668 | −14.529 | 1.00 | 50.00 | N |
| ATOM | 2747 | CA | ASN | B | 444 | −14.922 | 41.348 | −13.896 | 1.00 | 50.00 | C |
| ATOM | 2748 | C | ASN | B | 444 | −14.943 | 40.241 | −14.943 | 1.00 | 50.00 | C |
| ATOM | 2749 | O | ASN | B | 444 | −14.918 | 40.497 | −16.147 | 1.00 | 50.00 | O |
| ATOM | 2750 | CB | ASN | B | 444 | −16.084 | 41.304 | −12.900 | 1.00 | 50.00 | C |
| ATOM | 2751 | CG | ASN | B | 444 | −15.994 | 42.064 | −11.584 | 1.00 | 50.00 | C |
| ATOM | 2752 | OD1 | ASN | B | 444 | −17.008 | 42.358 | −10.954 | 1.00 | 50.00 | O |
| ATOM | 2753 | ND2 | ASN | B | 444 | −14.809 | 42.377 | −11.094 | 1.00 | 50.00 | N |
| ATOM | 2754 | N | LYS | B | 445 | −15.009 | 39.005 | −14.471 | 1.00 | 50.00 | N |
| ATOM | 2755 | CA | LYS | B | 445 | −15.455 | 37.816 | −15.199 | 1.00 | 50.00 | C |
| ATOM | 2756 | C | LYS | B | 445 | −16.325 | 38.064 | −16.428 | 1.00 | 50.00 | C |
| ATOM | 2757 | O | LYS | B | 445 | −15.791 | 38.094 | −17.537 | 1.00 | 50.00 | O |
| ATOM | 2758 | CB | LYS | B | 445 | −16.083 | 36.804 | −14.235 | 1.00 | 50.00 | C |
| ATOM | 2759 | CG | LYS | B | 445 | −15.156 | 35.995 | −13.329 | 1.00 | 50.00 | C |
| ATOM | 2760 | CD | LYS | B | 445 | −15.687 | 34.753 | −12.612 | 1.00 | 50.00 | C |
| ATOM | 2761 | CE | LYS | B | 445 | −14.626 | 33.947 | −11.864 | 1.00 | 50.00 | C |
| ATOM | 2762 | NZ | LYS | B | 445 | −14.970 | 33.048 | −10.753 | 1.00 | 50.00 | N |
| ATOM | 2763 | N | GLY | B | 446 | −17.613 | 38.315 | −16.262 | 1.00 | 50.00 | N |
| ATOM | 2764 | CA | GLY | B | 446 | −18.564 | 38.443 | −17.366 | 1.00 | 50.00 | C |
| ATOM | 2765 | C | GLY | B | 446 | −18.327 | 39.483 | −18.454 | 1.00 | 50.00 | C |
| ATOM | 2766 | O | GLY | B | 446 | −18.556 | 39.181 | −19.625 | 1.00 | 50.00 | O |
| ATOM | 2767 | N | VAL | B | 447 | −17.845 | 40.662 | −18.099 | 1.00 | 50.00 | N |
| ATOM | 2768 | CA | VAL | B | 447 | −17.421 | 41.704 | −19.037 | 1.00 | 50.00 | C |
| ATOM | 2769 | C | VAL | B | 447 | −16.256 | 41.244 | −19.910 | 1.00 | 50.00 | C |
| ATOM | 2770 | O | VAL | B | 447 | −16.403 | 41.111 | −21.125 | 1.00 | 50.00 | O |
| ATOM | 2771 | CB | VAL | B | 447 | −17.168 | 43.010 | −18.278 | 1.00 | 50.00 | C |
| ATOM | 2772 | CG1 | VAL | B | 447 | −16.508 | 44.099 | −19.124 | 1.00 | 50.00 | C |
| ATOM | 2773 | CG2 | VAL | B | 447 | −18.412 | 43.573 | −17.589 | 1.00 | 50.00 | C |
| ATOM | 2774 | N | ASP | B | 448 | −15.100 | 41.086 | −19.288 | 1.00 | 50.00 | N |
| ATOM | 2775 | CA | ASP | B | 448 | −13.799 | 40.922 | −19.934 | 1.00 | 50.00 | C |
| ATOM | 2776 | C | ASP | B | 448 | −13.134 | 39.707 | −19.299 | 1.00 | 50.00 | C |
| ATOM | 2777 | O | ASP | B | 448 | −13.237 | 39.445 | −18.101 | 1.00 | 50.00 | O |
| ATOM | 2778 | CB | ASP | B | 448 | −12.939 | 42.181 | −19.812 | 1.00 | 50.00 | C |
| ATOM | 2779 | CG | ASP | B | 448 | −12.634 | 42.671 | −18.403 | 1.00 | 50.00 | C |
| ATOM | 2780 | OD1 | ASP | B | 448 | −13.527 | 43.276 | −17.773 | 1.00 | 50.00 | O |
| ATOM | 2781 | OD2 | ASP | B | 448 | −11.522 | 42.406 | −17.898 | 1.00 | 50.00 | O |
| ATOM | 2782 | N | THR | B | 449 | −12.398 | 38.980 | −20.120 | 1.00 | 50.00 | N |
| ATOM | 2783 | CA | THR | B | 449 | −11.667 | 37.785 | −19.697 | 1.00 | 50.00 | C |
| ATOM | 2784 | C | THR | B | 449 | −10.185 | 38.004 | −19.413 | 1.00 | 50.00 | C |
| ATOM | 2785 | O | THR | B | 449 | −9.622 | 37.140 | −18.743 | 1.00 | 50.00 | O |
| ATOM | 2786 | CB | THR | B | 449 | −11.989 | 36.703 | −20.729 | 1.00 | 50.00 | C |
| ATOM | 2787 | OG1 | THR | B | 449 | −13.364 | 36.357 | −20.654 | 1.00 | 50.00 | O |
| ATOM | 2788 | CG2 | THR | B | 449 | −11.187 | 35.405 | −20.682 | 1.00 | 50.00 | C |
| ATOM | 2789 | N | VAL | B | 450 | −9.521 | 39.042 | −19.897 | 1.00 | 50.00 | N |
| ATOM | 2790 | CA | VAL | B | 450 | −8.073 | 39.220 | −19.779 | 1.00 | 50.00 | C |
| ATOM | 2791 | C | VAL | B | 450 | −7.824 | 40.726 | −19.813 | 1.00 | 50.00 | C |
| ATOM | 2792 | O | VAL | B | 450 | −7.593 | 41.299 | −20.877 | 1.00 | 50.00 | O |
| ATOM | 2793 | CB | VAL | B | 450 | −7.265 | 38.386 | −20.780 | 1.00 | 50.00 | C |
| ATOM | 2794 | CG1 | VAL | B | 450 | −7.214 | 36.874 | −20.563 | 1.00 | 50.00 | C |
| ATOM | 2795 | CG2 | VAL | B | 450 | −7.714 | 38.527 | −22.233 | 1.00 | 50.00 | C |
| ATOM | 2796 | N | SER | B | 451 | −7.849 | 41.414 | −18.682 | 1.00 | 50.00 | N |
| ATOM | 2797 | CA | SER | B | 451 | −7.499 | 42.832 | −18.578 | 1.00 | 50.00 | C |
| ATOM | 2798 | C | SER | B | 451 | −6.032 | 43.143 | −18.855 | 1.00 | 50.00 | C |
| ATOM | 2799 | O | SER | B | 451 | −5.151 | 42.301 | −18.683 | 1.00 | 50.00 | O |
| ATOM | 2800 | CB | SER | B | 451 | −7.865 | 43.371 | −17.195 | 1.00 | 50.00 | C |
| ATOM | 2801 | OG | SER | B | 451 | −9.267 | 43.448 | −16.990 | 1.00 | 50.00 | O |
| ATOM | 2802 | N | VAL | B | 452 | −5.774 | 44.365 | −19.292 | 1.00 | 50.00 | N |
| ATOM | 2803 | CA | VAL | B | 452 | −4.436 | 44.914 | −19.512 | 1.00 | 50.00 | C |
| ATOM | 2804 | C | VAL | B | 452 | −4.554 | 46.374 | −19.088 | 1.00 | 50.00 | C |
| ATOM | 2805 | O | VAL | B | 452 | −5.244 | 47.150 | −19.749 | 1.00 | 50.00 | O |
| ATOM | 2806 | CB | VAL | B | 452 | −3.894 | 44.713 | −20.931 | 1.00 | 50.00 | C |
| ATOM | 2807 | CG1 | VAL | B | 452 | −2.610 | 45.481 | −21.248 | 1.00 | 50.00 | C |
| ATOM | 2808 | CG2 | VAL | B | 452 | −3.627 | 43.253 | −21.296 | 1.00 | 50.00 | C |
| ATOM | 2809 | N | GLY | B | 453 | −3.867 | 46.768 | −18.028 | 1.00 | 50.00 | N |
| ATOM | 2810 | CA | GLY | B | 453 | −3.879 | 48.147 | −17.540 | 1.00 | 50.00 | C |
| ATOM | 2811 | C | GLY | B | 453 | −2.533 | 48.853 | −17.602 | 1.00 | 50.00 | C |
| ATOM | 2812 | O | GLY | B | 453 | −1.983 | 49.145 | −16.541 | 1.00 | 50.00 | O |
| ATOM | 2813 | N | ASN | B | 454 | −2.046 | 49.152 | −18.797 | 1.00 | 50.00 | N |
| ATOM | 2814 | CA | ASN | B | 454 | −0.827 | 49.923 | −19.050 | 1.00 | 50.00 | C |
| ATOM | 2815 | C | ASN | B | 454 | 0.373 | 49.508 | −18.207 | 1.00 | 50.00 | C |
| ATOM | 2816 | O | ASN | B | 454 | 0.584 | 50.143 | −17.174 | 1.00 | 50.00 | O |
| ATOM | 2817 | CB | ASN | B | 454 | −1.074 | 51.432 | −18.977 | 1.00 | 50.00 | C |
| ATOM | 2818 | CG | ASN | B | 454 | −1.875 | 52.120 | −20.074 | 1.00 | 50.00 | C |
| ATOM | 2819 | OD1 | ASN | B | 454 | −2.840 | 52.833 | −19.805 | 1.00 | 50.00 | O |
| ATOM | 2820 | ND2 | ASN | B | 454 | −1.463 | 51.989 | −21.322 | 1.00 | 50.00 | N |
| ATOM | 2821 | N | THR | B | 455 | 1.081 | 48.459 | −18.598 | 1.00 | 50.00 | N |
| ATOM | 2822 | CA | THR | B | 455 | 2.131 | 47.782 | −17.832 | 1.00 | 50.00 | C |
| ATOM | 2823 | C | THR | B | 455 | 1.778 | 46.312 | −17.622 | 1.00 | 50.00 | C |
| ATOM | 2824 | O | THR | B | 455 | 2.654 | 45.479 | −17.850 | 1.00 | 50.00 | O |
| ATOM | 2825 | CB | THR | B | 455 | 2.754 | 48.511 | −16.637 | 1.00 | 50.00 | C |
| ATOM | 2826 | OG1 | THR | B | 455 | 3.190 | 49.801 | −17.040 | 1.00 | 50.00 | O |
| ATOM | 2827 | CG2 | THR | B | 455 | 3.999 | 47.975 | −15.934 | 1.00 | 50.00 | C |
| ATOM | 2828 | N | LEU | B | 456 | 0.594 | 45.951 | −17.150 | 1.00 | 50.00 | N |
| ATOM | 2829 | CA | LEU | B | 456 | 0.294 | 44.633 | −16.588 | 1.00 | 50.00 | C |
| ATOM | 2830 | C | LEU | B | 456 | −0.774 | 43.929 | −17.410 | 1.00 | 50.00 | C |
| ATOM | 2831 | O | LEU | B | 456 | −1.663 | 44.565 | −17.974 | 1.00 | 50.00 | O |
| ATOM | 2832 | CB | LEU | B | 456 | −0.252 | 44.696 | −15.160 | 1.00 | 50.00 | C |
| ATOM | 2833 | CG | LEU | B | 456 | 0.805 | 44.997 | −14.103 | 1.00 | 50.00 | C |
| ATOM | 2834 | CD1 | LEU | B | 456 | 1.391 | 46.402 | −14.185 | 1.00 | 50.00 | C |
| ATOM | 2835 | CD2 | LEU | B | 456 | 0.368 | 44.696 | −12.675 | 1.00 | 50.00 | C |
| ATOM | 2836 | N | TYR | B | 457 | −0.732 | 42.609 | −17.349 | 1.00 | 50.00 | N |
| ATOM | 2837 | CA | TYR | B | 457 | −1.850 | 41.756 | −17.746 | 1.00 | 50.00 | C |
| ATOM | 2838 | C | TYR | B | 457 | −2.444 | 41.230 | −16.444 | 1.00 | 50.00 | C |
| ATOM | 2839 | O | TYR | B | 457 | −1.765 | 40.671 | −15.583 | 1.00 | 50.00 | O |
| ATOM | 2840 | CB | TYR | B | 457 | −1.356 | 40.798 | −18.834 | 1.00 | 50.00 | C |
| ATOM | 2841 | CG | TYR | B | 457 | −1.449 | 39.296 | −18.672 | 1.00 | 50.00 | C |
| ATOM | 2842 | CD1 | TYR | B | 457 | −0.978 | 38.704 | −17.555 | 1.00 | 50.00 | C |
| ATOM | 2843 | CD2 | TYR | B | 457 | −1.930 | 38.524 | −19.669 | 1.00 | 50.00 | C |
| ATOM | 2844 | CE1 | TYR | B | 457 | −1.104 | 37.375 | −17.374 | 1.00 | 50.00 | C |
| ATOM | 2845 | CE2 | TYR | B | 457 | −1.969 | 37.183 | −19.537 | 1.00 | 50.00 | C |
| ATOM | 2846 | CZ | TYR | B | 457 | −1.542 | 36.609 | −18.393 | 1.00 | 50.00 | C |
| ATOM | 2847 | OH | TYR | B | 457 | −1.622 | 35.253 | −18.222 | 1.00 | 50.00 | O |
| ATOM | 2848 | N | TYR | B | 458 | −3.749 | 41.364 | −16.293 | 1.00 | 50.00 | N |
| ATOM | 2849 | CA | TYR | B | 458 | −4.534 | 40.509 | −15.404 | 1.00 | 50.00 | C |
| ATOM | 2850 | C | TYR | B | 458 | −5.349 | 39.527 | −16.238 | 1.00 | 50.00 | C |
| ATOM | 2851 | O | TYR | B | 458 | −5.943 | 39.914 | −17.243 | 1.00 | 50.00 | O |
| ATOM | 2852 | CB | TYR | B | 458 | −5.325 | 41.449 | −14.493 | 1.00 | 50.00 | C |
| ATOM | 2853 | CG | TYR | B | 458 | −6.038 | 40.707 | −13.387 | 1.00 | 50.00 | C |
| ATOM | 2854 | CD1 | TYR | B | 458 | −5.251 | 40.056 | −12.506 | 1.00 | 50.00 | C |
| ATOM | 2855 | CD2 | TYR | B | 458 | −7.348 | 40.399 | −13.485 | 1.00 | 50.00 | C |
| ATOM | 2856 | CE1 | TYR | B | 458 | −5.740 | 38.989 | −11.847 | 1.00 | 50.00 | C |
| ATOM | 2857 | CE2 | TYR | B | 458 | −7.852 | 39.372 | −12.772 | 1.00 | 50.00 | C |
| ATOM | 2858 | CZ | TYR | B | 458 | −7.028 | 38.631 | −12.008 | 1.00 | 50.00 | C |
| ATOM | 2859 | OH | TYR | B | 458 | −7.480 | 37.481 | −11.423 | 1.00 | 50.00 | O |
| ATOM | 2860 | N | VAL | B | 459 | −5.428 | 38.269 | −15.837 | 1.00 | 50.00 | N |
| ATOM | 2861 | CA | VAL | B | 459 | −6.343 | 37.296 | −16.438 | 1.00 | 50.00 | C |
| ATOM | 2862 | C | VAL | B | 459 | −7.617 | 37.326 | −15.602 | 1.00 | 50.00 | C |
| ATOM | 2863 | O | VAL | B | 459 | −7.596 | 36.920 | −14.442 | 1.00 | 50.00 | O |
| ATOM | 2864 | CB | VAL | B | 459 | −5.669 | 35.933 | −16.612 | 1.00 | 50.00 | C |
| ATOM | 2865 | CG1 | VAL | B | 459 | −6.584 | 34.730 | −16.834 | 1.00 | 50.00 | C |
| ATOM | 2866 | CG2 | VAL | B | 459 | −4.600 | 35.909 | −17.700 | 1.00 | 50.00 | C |
| ATOM | 2867 | N | ASN | B | 460 | −8.752 | 37.761 | −16.122 | 1.00 | 50.00 | N |
| ATOM | 2868 | CA | ASN | B | 460 | −10.045 | 37.515 | −15.481 | 1.00 | 50.00 | C |
| ATOM | 2869 | C | ASN | B | 460 | −10.713 | 36.194 | −15.864 | 1.00 | 50.00 | C |
| ATOM | 2870 | O | ASN | B | 460 | −11.902 | 36.169 | −15.552 | 1.00 | 50.00 | O |
| ATOM | 2871 | CB | ASN | B | 460 | −10.962 | 38.736 | −15.599 | 1.00 | 50.00 | C |
| ATOM | 2872 | CG | ASN | B | 460 | −10.608 | 40.106 | −15.034 | 1.00 | 50.00 | C |
| ATOM | 2873 | OD1 | ASN | B | 460 | −10.165 | 40.989 | −15.766 | 1.00 | 50.00 | O |
| ATOM | 2874 | ND2 | ASN | B | 460 | −10.845 | 40.386 | −13.765 | 1.00 | 50.00 | N |
| ATOM | 2875 | N | LYS | B | 461 | −10.134 | 35.112 | −16.373 | 1.00 | 50.00 | N |
| ATOM | 2876 | CA | LYS | B | 461 | −10.757 | 33.789 | −16.517 | 1.00 | 50.00 | C |
| ATOM | 2877 | C | LYS | B | 461 | −9.835 | 32.581 | −16.660 | 1.00 | 50.00 | C |
| ATOM | 2878 | O | LYS | B | 461 | −8.994 | 32.586 | −17.558 | 1.00 | 50.00 | O |
| ATOM | 2879 | CB | LYS | B | 461 | −11.984 | 33.510 | −17.387 | 1.00 | 50.00 | C |
| ATOM | 2880 | CG | LYS | B | 461 | −13.255 | 34.270 | −17.020 | 1.00 | 50.00 | C |
| ATOM | 2881 | CD | LYS | B | 461 | −14.501 | 33.959 | −17.843 | 1.00 | 50.00 | C |
| ATOM | 2882 | CE | LYS | B | 461 | −15.609 | 34.868 | −17.318 | 1.00 | 50.00 | C |
| ATOM | 2883 | NZ | LYS | B | 461 | −16.726 | 35.026 | −18.260 | 1.00 | 50.00 | N |
| ATOM | 2884 | N | GLN | B | 462 | −10.059 | 31.533 | −15.880 | 1.00 | 50.00 | N |
| ATOM | 2885 | CA | GLN | B | 462 | −9.336 | 30.260 | −15.828 | 1.00 | 50.00 | C |
| ATOM | 2886 | C | GLN | B | 462 | −10.033 | 29.253 | −14.910 | 1.00 | 50.00 | C |
| ATOM | 2887 | O | GLN | B | 462 | −10.647 | 29.629 | −13.912 | 1.00 | 50.00 | O |
| ATOM | 2888 | CB | GLN | B | 462 | −7.883 | 30.445 | −15.382 | 1.00 | 50.00 | C |
| ATOM | 2889 | CG | GLN | B | 462 | −6.780 | 31.166 | −16.150 | 1.00 | 50.00 | C |
| ATOM | 2890 | CD | GLN | B | 462 | −6.582 | 30.695 | −17.582 | 1.00 | 50.00 | C |
| ATOM | 2891 | OE1 | GLN | B | 462 | −6.781 | 29.532 | −17.931 | 1.00 | 50.00 | O |
| ATOM | 2892 | NE2 | GLN | B | 462 | −6.217 | 31.619 | −18.450 | 1.00 | 50.00 | N |
| ATOM | 2893 | N | GLU | B | 463 | −9.904 | 27.955 | −15.149 | 1.00 | 50.00 | N |
| ATOM | 2894 | CA | GLU | B | 463 | −10.269 | 26.863 | −14.239 | 1.00 | 50.00 | C |
| ATOM | 2895 | C | GLU | B | 463 | −9.268 | 25.709 | −14.251 | 1.00 | 50.00 | C |
| ATOM | 2896 | O | GLU | B | 463 | −8.399 | 25.615 | −15.118 | 1.00 | 50.00 | O |
| ATOM | 2897 | CB | GLU | B | 463 | −11.667 | 26.322 | −14.548 | 1.00 | 50.00 | C |
| ATOM | 2898 | CG | GLU | B | 463 | −12.829 | 26.986 | −13.812 | 1.00 | 50.00 | C |
| ATOM | 2899 | CD | GLU | B | 463 | −14.179 | 26.914 | −14.511 | 1.00 | 50.00 | C |
| ATOM | 2900 | OE1 | GLU | B | 463 | −14.252 | 26.634 | −15.728 | 1.00 | 50.00 | O |
| ATOM | 2901 | OE2 | GLU | B | 463 | −15.201 | 27.165 | −13.836 | 1.00 | 50.00 | O |
| ATOM | 2902 | N | GLY | B | 464 | −9.367 | 24.813 | −13.281 | 1.00 | 50.00 | N |
| ATOM | 2903 | CA | GLY | B | 464 | −8.520 | 23.628 | −13.143 | 1.00 | 50.00 | C |
| ATOM | 2904 | C | GLY | B | 464 | −9.188 | 22.544 | −12.308 | 1.00 | 50.00 | C |
| ATOM | 2905 | O | GLY | B | 464 | −9.936 | 22.819 | −11.371 | 1.00 | 50.00 | O |
| ATOM | 2906 | N | LYS | B | 465 | −8.921 | 21.297 | −12.658 | 1.00 | 50.00 | N |
| ATOM | 2907 | CA | LYS | B | 465 | −9.589 | 20.135 | −12.071 | 1.00 | 50.00 | C |
| ATOM | 2908 | C | LYS | B | 465 | −8.611 | 18.971 | −11.974 | 1.00 | 50.00 | C |
| ATOM | 2909 | O | LYS | B | 465 | −7.764 | 18.770 | −12.844 | 1.00 | 50.00 | O |
| ATOM | 2910 | CB | LYS | B | 465 | −10.825 | 19.727 | −12.874 | 1.00 | 50.00 | C |
| ATOM | 2911 | CG | LYS | B | 465 | −11.964 | 20.746 | −12.874 | 1.00 | 50.00 | C |
| ATOM | 2912 | CD | LYS | B | 465 | −13.180 | 20.303 | −13.684 | 1.00 | 50.00 | C |
| ATOM | 2913 | CE | LYS | B | 465 | −14.236 | 21.404 | −13.755 | 1.00 | 50.00 | C |
| ATOM | 2914 | NZ | LYS | B | 465 | −15.408 | 20.956 | −14.522 | 1.00 | 50.00 | N |
| ATOM | 2915 | N | SER | B | 466 | −8.743 | 18.198 | −10.908 | 1.00 | 50.00 | N |
| ATOM | 2916 | CA | SER | B | 466 | −7.854 | 17.080 | −10.589 | 1.00 | 50.00 | C |
| ATOM | 2917 | C | SER | B | 466 | −8.447 | 16.162 | −9.528 | 1.00 | 50.00 | C |
| ATOM | 2918 | O | SER | B | 466 | −9.286 | 16.540 | −8.711 | 1.00 | 50.00 | O |
| ATOM | 2919 | CB | SER | B | 466 | −6.507 | 17.564 | −10.058 | 1.00 | 50.00 | C |
| ATOM | 2920 | OG | SER | B | 466 | −5.750 | 18.092 | −11.137 | 1.00 | 50.00 | O |
| ATOM | 2921 | N | LEU | B | 467 | −7.992 | 14.922 | −9.585 | 1.00 | 50.00 | N |
| ATOM | 2922 | CA | LEU | B | 467 | −8.507 | 13.818 | −8.776 | 1.00 | 50.00 | C |
| ATOM | 2923 | C | LEU | B | 467 | −7.321 | 13.186 | −8.054 | 1.00 | 50.00 | C |
| ATOM | 2924 | O | LEU | B | 467 | −6.331 | 12.812 | −8.683 | 1.00 | 50.00 | O |
| ATOM | 2925 | CB | LEU | B | 467 | −9.270 | 12.853 | −9.685 | 1.00 | 50.00 | C |
| ATOM | 2926 | CG | LEU | B | 467 | −10.649 | 13.249 | −10.217 | 1.00 | 50.00 | C |
| ATOM | 2927 | CD1 | LEU | B | 467 | −10.716 | 14.356 | −11.270 | 1.00 | 50.00 | C |
| ATOM | 2928 | CD2 | LEU | B | 467 | −11.351 | 12.036 | −10.826 | 1.00 | 50.00 | C |
| ATOM | 2929 | N | TYR | B | 468 | −7.415 | 13.058 | −6.740 | 1.00 | 50.00 | N |
| ATOM | 2930 | CA | TYR | B | 468 | −6.413 | 12.403 | −5.896 | 1.00 | 50.00 | C |
| ATOM | 2931 | C | TYR | B | 468 | −6.938 | 11.018 | −5.539 | 1.00 | 50.00 | C |
| ATOM | 2932 | O | TYR | B | 468 | −7.925 | 10.910 | −4.811 | 1.00 | 50.00 | O |
| ATOM | 2933 | CB | TYR | B | 468 | −6.117 | 13.223 | −4.638 | 1.00 | 50.00 | C |
| ATOM | 2934 | CG | TYR | B | 468 | −4.852 | 12.883 | −3.875 | 1.00 | 50.00 | C |
| ATOM | 2935 | CD1 | TYR | B | 468 | −3.864 | 13.800 | −3.830 | 1.00 | 50.00 | C |
| ATOM | 2936 | CD2 | TYR | B | 468 | −4.596 | 11.633 | −3.433 | 1.00 | 50.00 | C |
| ATOM | 2937 | CE1 | TYR | B | 468 | −2.595 | 13.427 | −3.566 | 1.00 | 50.00 | C |
| ATOM | 2938 | CE2 | TYR | B | 468 | −3.333 | 11.259 | −3.146 | 1.00 | 50.00 | C |
| ATOM | 2939 | CZ | TYR | B | 468 | −2.322 | 12.139 | −3.275 | 1.00 | 50.00 | C |
| ATOM | 2940 | OH | TYR | B | 468 | −1.030 | 11.691 | −3.183 | 1.00 | 50.00 | O |
| ATOM | 2941 | N | VAL | B | 469 | −6.290 | 9.969 | −6.021 | 1.00 | 50.00 | N |
| ATOM | 2942 | CA | VAL | B | 469 | −6.756 | 8.589 | −5.874 | 1.00 | 50.00 | C |
| ATOM | 2943 | C | VAL | B | 469 | −5.568 | 7.633 | −5.784 | 1.00 | 50.00 | C |
| ATOM | 2944 | O | VAL | B | 469 | −4.506 | 7.907 | −6.341 | 1.00 | 50.00 | O |
| ATOM | 2945 | CB | VAL | B | 469 | −7.803 | 8.176 | −6.915 | 1.00 | 50.00 | C |
| ATOM | 2946 | CG1 | VAL | B | 469 | −8.895 | 9.192 | −7.249 | 1.00 | 50.00 | C |
| ATOM | 2947 | CG2 | VAL | B | 469 | −7.282 | 7.639 | −8.248 | 1.00 | 50.00 | C |
| ATOM | 2948 | N | LYS | B | 470 | −5.759 | 6.464 | −5.193 | 1.00 | 50.00 | N |
| ATOM | 2949 | CA | LYS | B | 470 | −4.864 | 5.317 | −5.359 | 1.00 | 50.00 | C |
| ATOM | 2950 | C | LYS | B | 470 | −5.508 | 4.120 | −6.051 | 1.00 | 50.00 | C |
| ATOM | 2951 | O | LYS | B | 470 | −6.711 | 3.886 | −5.945 | 1.00 | 50.00 | O |
| ATOM | 2952 | CB | LYS | B | 470 | −4.295 | 4.853 | −4.018 | 1.00 | 50.00 | C |
| ATOM | 2953 | CG | LYS | B | 470 | −2.946 | 4.161 | −4.204 | 1.00 | 50.00 | C |
| ATOM | 2954 | CD | LYS | B | 470 | −2.271 | 3.707 | −2.916 | 1.00 | 50.00 | C |
| ATOM | 2955 | CE | LYS | B | 470 | −0.846 | 3.282 | −3.270 | 1.00 | 50.00 | C |
| ATOM | 2956 | NZ | LYS | B | 470 | 0.004 | 2.948 | −2.121 | 1.00 | 50.00 | N |
| ATOM | 2957 | N | GLY | B | 471 | −4.666 | 3.306 | −6.666 | 1.00 | 50.00 | N |
| ATOM | 2958 | CA | GLY | B | 471 | −4.955 | 1.901 | −6.953 | 1.00 | 50.00 | C |
| ATOM | 2959 | C | GLY | B | 471 | −4.015 | 0.935 | −6.244 | 1.00 | 50.00 | C |
| ATOM | 2960 | O | GLY | B | 471 | −2.824 | 0.915 | −6.556 | 1.00 | 50.00 | O |
| ATOM | 2961 | N | GLU | B | 472 | −4.548 | 0.090 | −5.375 | 1.00 | 50.00 | N |
| ATOM | 2962 | CA | GLU | B | 472 | −3.841 | −1.051 | −4.790 | 1.00 | 50.00 | C |
| ATOM | 2963 | C | GLU | B | 472 | −3.308 | −2.075 | −5.786 | 1.00 | 50.00 | C |
| ATOM | 2964 | O | GLU | B | 472 | −4.103 | −2.512 | −6.617 | 1.00 | 50.00 | O |
| ATOM | 2965 | CB | GLU | B | 472 | −4.614 | −1.662 | −3.618 | 1.00 | 50.00 | C |
| ATOM | 2966 | CG | GLU | B | 472 | −4.751 | −3.168 | −3.390 | 1.00 | 50.00 | C |
| ATOM | 2967 | CD | GLU | B | 472 | −5.741 | −3.855 | −4.319 | 1.00 | 50.00 | C |
| ATOM | 2968 | OE1 | GLU | B | 472 | −5.303 | −4.630 | −5.196 | 1.00 | 50.00 | O |
| ATOM | 2969 | OE2 | GLU | B | 472 | −6.965 | −3.632 | −4.195 | 1.00 | 50.00 | O |
| ATOM | 2970 | N | PRO | B | 473 | −2.071 | −2.542 | −5.681 | 1.00 | 50.00 | N |
| ATOM | 2971 | CA | PRO | B | 473 | −1.630 | −3.754 | −6.370 | 1.00 | 50.00 | C |
| ATOM | 2972 | C | PRO | B | 473 | −1.261 | −4.877 | −5.406 | 1.00 | 50.00 | C |
| ATOM | 2973 | O | PRO | B | 473 | −0.183 | −4.867 | −4.813 | 1.00 | 50.00 | O |
| ATOM | 2974 | CB | PRO | B | 473 | −0.469 | −3.174 | −7.176 | 1.00 | 50.00 | C |
| ATOM | 2975 | CG | PRO | B | 473 | 0.178 | −2.120 | −6.281 | 1.00 | 50.00 | C |
| ATOM | 2976 | CD | PRO | B | 473 | −0.931 | −1.698 | −5.321 | 1.00 | 50.00 | C |
| ATOM | 2977 | N | ILE | B | 474 | −2.128 | −5.866 | −5.267 | 1.00 | 50.00 | N |
| ATOM | 2978 | CA | ILE | B | 474 | −1.849 | −7.095 | −4.520 | 1.00 | 50.00 | C |
| ATOM | 2979 | C | ILE | B | 474 | −0.922 | −8.011 | −5.314 | 1.00 | 50.00 | C |
| ATOM | 2980 | O | ILE | B | 474 | −1.133 | −8.224 | −6.507 | 1.00 | 50.00 | O |
| ATOM | 2981 | CB | ILE | B | 474 | −3.115 | −7.812 | −4.043 | 1.00 | 50.00 | C |
| ATOM | 2982 | CG1 | ILE | B | 474 | −4.024 | −8.346 | −5.152 | 1.00 | 50.00 | C |
| ATOM | 2983 | CG2 | ILE | B | 474 | −3.897 | −6.994 | −3.014 | 1.00 | 50.00 | C |
| ATOM | 2984 | CD1 | ILE | B | 474 | −5.074 | −9.364 | −4.716 | 1.00 | 50.00 | C |
| ATOM | 2985 | N | ILE | B | 475 | 0.089 | −8.571 | −4.668 | 1.00 | 50.00 | N |
| ATOM | 2986 | CA | ILE | B | 475 | 0.992 | −9.556 | −5.266 | 1.00 | 50.00 | C |
| ATOM | 2987 | C | ILE | B | 475 | 0.671 | −10.879 | −4.579 | 1.00 | 50.00 | C |
| ATOM | 2988 | O | ILE | B | 475 | 0.913 | −11.027 | −3.381 | 1.00 | 50.00 | O |
| ATOM | 2989 | CB | ILE | B | 475 | 2.466 | −9.150 | −5.183 | 1.00 | 50.00 | C |
| ATOM | 2990 | CG1 | ILE | B | 475 | 2.795 | −7.830 | −5.884 | 1.00 | 50.00 | C |
| ATOM | 2991 | CG2 | ILE | B | 475 | 3.397 | −10.241 | −5.716 | 1.00 | 50.00 | C |
| ATOM | 2992 | CD1 | ILE | B | 475 | 4.131 | −7.189 | −5.510 | 1.00 | 50.00 | C |
| ATOM | 2993 | N | ASN | B | 476 | 0.189 | −11.850 | −5.339 | 1.00 | 50.00 | N |
| ATOM | 2994 | CA | ASN | B | 476 | −0.050 | −13.212 | −4.862 | 1.00 | 50.00 | C |
| ATOM | 2995 | C | ASN | B | 476 | 0.814 | −14.196 | −5.642 | 1.00 | 50.00 | C |
| ATOM | 2996 | O | ASN | B | 476 | 0.600 | −14.402 | −6.837 | 1.00 | 50.00 | O |
| ATOM | 2997 | CB | ASN | B | 476 | −1.532 | −13.581 | −4.950 | 1.00 | 50.00 | C |
| ATOM | 2998 | CG | ASN | B | 476 | −2.515 | −12.871 | −4.028 | 1.00 | 50.00 | C |
| ATOM | 2999 | OD1 | ASN | B | 476 | −3.619 | −12.516 | −4.436 | 1.00 | 50.00 | O |
| ATOM | 3000 | ND2 | ASN | B | 476 | −2.179 | −12.676 | −2.766 | 1.00 | 50.00 | N |
| ATOM | 3001 | N | PHE | B | 477 | 1.754 | −14.826 | −4.956 | 1.00 | 50.00 | N |
| ATOM | 3002 | CA | PHE | B | 477 | 2.510 | −15.970 | −5.466 | 1.00 | 50.00 | C |
| ATOM | 3003 | C | PHE | B | 477 | 2.004 | −17.234 | −4.779 | 1.00 | 50.00 | C |
| ATOM | 3004 | O | PHE | B | 477 | 2.004 | −17.333 | −3.552 | 1.00 | 50.00 | O |
| ATOM | 3005 | CB | PHE | B | 477 | 4.012 | −15.757 | −5.272 | 1.00 | 50.00 | C |
| ATOM | 3006 | CG | PHE | B | 477 | 4.898 | −16.803 | −5.915 | 1.00 | 50.00 | C |
| ATOM | 3007 | CD1 | PHE | B | 477 | 5.109 | −16.787 | −7.248 | 1.00 | 50.00 | C |
| ATOM | 3008 | CD2 | PHE | B | 477 | 5.419 | −17.806 | −5.176 | 1.00 | 50.00 | C |
| ATOM | 3009 | CE1 | PHE | B | 477 | 5.817 | −17.770 | −7.840 | 1.00 | 50.00 | C |
| ATOM | 3010 | CE2 | PHE | B | 477 | 6.128 | −18.791 | −5.763 | 1.00 | 50.00 | C |
| ATOM | 3011 | CZ | PHE | B | 477 | 6.326 | −18.773 | −7.097 | 1.00 | 50.00 | C |
| ATOM | 3012 | N | TYR | B | 478 | 1.630 | −18.211 | −5.588 | 1.00 | 50.00 | N |
| ATOM | 3013 | CA | TYR | B | 478 | 1.214 | −19.537 | −5.134 | 1.00 | 50.00 | C |
| ATOM | 3014 | C | TYR | B | 478 | 2.142 | −20.588 | −5.732 | 1.00 | 50.00 | C |
| ATOM | 3015 | O | TYR | B | 478 | 2.373 | −20.624 | −6.940 | 1.00 | 50.00 | O |
| ATOM | 3016 | CB | TYR | B | 478 | −0.255 | −19.749 | −5.505 | 1.00 | 50.00 | C |
| ATOM | 3017 | CG | TYR | B | 478 | −0.840 | −21.068 | −5.050 | 1.00 | 50.00 | C |
| ATOM | 3018 | CD1 | TYR | B | 478 | −1.117 | −21.235 | −3.741 | 1.00 | 50.00 | C |
| ATOM | 3019 | CD2 | TYR | B | 478 | −0.971 | −22.110 | −5.898 | 1.00 | 50.00 | C |
| ATOM | 3020 | CE1 | TYR | B | 478 | −1.518 | −22.438 | −3.287 | 1.00 | 50.00 | C |
| ATOM | 3021 | CE2 | TYR | B | 478 | −1.361 | −23.317 | −5.439 | 1.00 | 50.00 | C |
| ATOM | 3022 | CZ | TYR | B | 478 | −1.636 | −23.484 | −4.130 | 1.00 | 50.00 | C |
| ATOM | 3023 | OH | TYR | B | 478 | −2.034 | −24.721 | −3.696 | 1.00 | 50.00 | O |
| ATOM | 3024 | N | ASP | B | 479 | 2.593 | −21.493 | −4.879 | 1.00 | 50.00 | N |
| ATOM | 3025 | CA | ASP | B | 479 | 3.262 | −22.730 | −5.282 | 1.00 | 50.00 | C |
| ATOM | 3026 | C | ASP | B | 479 | 2.327 | −23.920 | −5.098 | 1.00 | 50.00 | C |
| ATOM | 3027 | O | ASP | B | 479 | 1.790 | −24.058 | −3.999 | 1.00 | 50.00 | O |
| ATOM | 3028 | CB | ASP | B | 479 | 4.595 | −22.927 | −4.558 | 1.00 | 50.00 | C |
| ATOM | 3029 | CG | ASP | B | 479 | 4.591 | −23.156 | −3.053 | 1.00 | 50.00 | C |
| ATOM | 3030 | OD1 | ASP | B | 479 | 4.408 | −22.181 | −2.292 | 1.00 | 50.00 | O |
| ATOM | 3031 | OD2 | ASP | B | 479 | 4.754 | −24.317 | −2.618 | 1.00 | 50.00 | O |
| ATOM | 3032 | N | PRO | B | 480 | 2.164 | −24.851 | −6.027 | 1.00 | 50.00 | N |
| ATOM | 3033 | CA | PRO | B | 480 | 1.643 | −26.181 | −5.714 | 1.00 | 50.00 | C |
| ATOM | 3034 | C | PRO | B | 480 | 2.703 | −27.009 | −4.995 | 1.00 | 50.00 | C |
| ATOM | 3035 | O | PRO | B | 480 | 3.675 | −27.483 | −5.582 | 1.00 | 50.00 | O |
| ATOM | 3036 | CB | PRO | B | 480 | 1.205 | −26.678 | −7.090 | 1.00 | 50.00 | C |
| ATOM | 3037 | CG | PRO | B | 480 | 2.173 | −26.043 | −8.084 | 1.00 | 50.00 | C |
| ATOM | 3038 | CD | PRO | B | 480 | 2.513 | −24.701 | −7.440 | 1.00 | 50.00 | C |
| ATOM | 3039 | N | LEU | B | 481 | 2.491 | −27.223 | −3.707 | 1.00 | 50.00 | N |
| ATOM | 3040 | CA | LEU | B | 481 | 2.796 | −28.487 | −3.036 | 1.00 | 50.00 | C |
| ATOM | 3041 | C | LEU | B | 481 | 1.993 | −29.612 | −3.682 | 1.00 | 50.00 | C |
| ATOM | 3042 | O | LEU | B | 481 | 0.764 | −29.624 | −3.611 | 1.00 | 50.00 | O |
| ATOM | 3043 | CB | LEU | B | 481 | 2.477 | −28.367 | −1.544 | 1.00 | 50.00 | C |
| ATOM | 3044 | CG | LEU | B | 481 | 3.507 | −27.671 | −0.654 | 1.00 | 50.00 | C |
| ATOM | 3045 | CD1 | LEU | B | 481 | 2.940 | −27.389 | 0.736 | 1.00 | 50.00 | C |
| ATOM | 3046 | CD2 | LEU | B | 481 | 4.809 | −28.460 | −0.520 | 1.00 | 50.00 | C |
| ATOM | 3047 | N | VAL | B | 482 | 2.689 | −30.484 | −4.393 | 1.00 | 50.00 | N |
| ATOM | 3048 | CA | VAL | B | 482 | 2.080 | −31.477 | −5.278 | 1.00 | 50.00 | C |
| ATOM | 3049 | C | VAL | B | 482 | 2.299 | −32.804 | −4.562 | 1.00 | 50.00 | C |
| ATOM | 3050 | O | VAL | B | 482 | 3.401 | −33.352 | −4.580 | 1.00 | 50.00 | O |
| ATOM | 3051 | CB | VAL | B | 482 | 2.626 | −31.478 | −6.709 | 1.00 | 50.00 | C |
| ATOM | 3052 | CG1 | VAL | B | 482 | 1.908 | −32.460 | −7.634 | 1.00 | 50.00 | C |
| ATOM | 3053 | CG2 | VAL | B | 482 | 2.558 | −30.131 | −7.426 | 1.00 | 50.00 | C |
| ATOM | 3054 | N | PHE | B | 483 | 1.232 | −33.353 | −4.004 | 1.00 | 50.00 | N |
| ATOM | 3055 | CA | PHE | B | 483 | 1.196 | −34.745 | −3.555 | 1.00 | 50.00 | C |
| ATOM | 3056 | C | PHE | B | 483 | 1.443 | −35.700 | −4.722 | 1.00 | 50.00 | C |
| ATOM | 3057 | O | PHE | B | 483 | 0.743 | −35.517 | −5.718 | 1.00 | 50.00 | O |
| ATOM | 3058 | CB | PHE | B | 483 | −0.121 | −35.035 | −2.827 | 1.00 | 50.00 | C |
| ATOM | 3059 | CG | PHE | B | 483 | −0.056 | −36.207 | −1.869 | 1.00 | 50.00 | C |
| ATOM | 3060 | CD1 | PHE | B | 483 | 0.239 | −35.936 | −0.582 | 1.00 | 50.00 | C |
| ATOM | 3061 | CD2 | PHE | B | 483 | 0.243 | −37.433 | −2.348 | 1.00 | 50.00 | C |
| ATOM | 3062 | CE1 | PHE | B | 483 | 1.063 | −36.753 | 0.103 | 1.00 | 50.00 | C |
| ATOM | 3063 | CE2 | PHE | B | 483 | 1.136 | −38.208 | −1.701 | 1.00 | 50.00 | C |
| ATOM | 3064 | CZ | PHE | B | 483 | 1.650 | −37.789 | −0.527 | 1.00 | 50.00 | C |
| ATOM | 3065 | N | PRO | B | 484 | 2.305 | −36.708 | −4.697 | 1.00 | 50.00 | N |
| ATOM | 3066 | CA | PRO | B | 484 | 2.298 | −37.799 | −5.675 | 1.00 | 50.00 | C |
| ATOM | 3067 | C | PRO | B | 484 | 1.100 | −38.743 | −5.603 | 1.00 | 50.00 | C |
| ATOM | 3068 | O | PRO | B | 484 | 1.216 | −39.914 | −5.242 | 1.00 | 50.00 | O |
| ATOM | 3069 | CB | PRO | B | 484 | 3.643 | −38.471 | −5.396 | 1.00 | 50.00 | C |
| ATOM | 3070 | CG | PRO | B | 484 | 3.855 | −38.287 | −3.896 | 1.00 | 50.00 | C |
| ATOM | 3071 | CD | PRO | B | 484 | 3.373 | −36.852 | −3.708 | 1.00 | 50.00 | C |
| ATOM | 3072 | N | SER | B | 485 | −0.076 | −38.234 | −5.932 | 1.00 | 50.00 | N |
| ATOM | 3073 | CA | SER | B | 485 | −1.368 | −38.861 | −5.650 | 1.00 | 50.00 | C |
| ATOM | 3074 | C | SER | B | 485 | −1.759 | −40.016 | −6.564 | 1.00 | 50.00 | C |
| ATOM | 3075 | O | SER | B | 485 | −2.298 | −41.003 | −6.066 | 1.00 | 50.00 | O |
| ATOM | 3076 | CB | SER | B | 485 | −2.456 | −37.786 | −5.619 | 1.00 | 50.00 | C |
| ATOM | 3077 | OG | SER | B | 485 | −2.518 | −37.157 | −4.347 | 1.00 | 50.00 | O |
| ATOM | 3078 | N | ASP | B | 486 | −1.514 | −39.917 | −7.861 | 1.00 | 50.00 | N |
| ATOM | 3079 | CA | ASP | B | 486 | −1.808 | −40.967 | −8.836 | 1.00 | 50.00 | C |
| ATOM | 3080 | C | ASP | B | 486 | −0.944 | −42.220 | −8.728 | 1.00 | 50.00 | C |
| ATOM | 3081 | O | ASP | B | 486 | −1.512 | −43.307 | −8.824 | 1.00 | 50.00 | O |
| ATOM | 3082 | CB | ASP | B | 486 | −1.808 | −40.388 | −10.252 | 1.00 | 50.00 | C |
| ATOM | 3083 | CG | ASP | B | 486 | −2.831 | −39.295 | −10.528 | 1.00 | 50.00 | C |
| ATOM | 3084 | OD1 | ASP | B | 486 | −4.034 | −39.608 | −10.657 | 1.00 | 50.00 | O |
| ATOM | 3085 | OD2 | ASP | B | 486 | −2.442 | −38.109 | −10.597 | 1.00 | 50.00 | O |
| ATOM | 3086 | N | GLU | B | 487 | 0.352 | −42.130 | −8.469 | 1.00 | 50.00 | N |
| ATOM | 3087 | CA | GLU | B | 487 | 1.160 | −43.247 | −7.975 | 1.00 | 50.00 | C |
| ATOM | 3088 | C | GLU | B | 487 | 0.642 | −43.862 | −6.680 | 1.00 | 50.00 | C |
| ATOM | 3089 | O | GLU | B | 487 | 0.436 | −45.075 | −6.667 | 1.00 | 50.00 | O |
| ATOM | 3090 | CB | GLU | B | 487 | 2.646 | −42.901 | −7.858 | 1.00 | 50.00 | C |
| ATOM | 3091 | CG | GLU | B | 487 | 3.603 | −44.060 | −7.570 | 1.00 | 50.00 | C |
| ATOM | 3092 | CD | GLU | B | 487 | 3.918 | −44.358 | −6.110 | 1.00 | 50.00 | C |
| ATOM | 3093 | OE1 | GLU | B | 487 | 3.093 | −44.982 | −5.409 | 1.00 | 50.00 | O |
| ATOM | 3094 | OE2 | GLU | B | 487 | 5.020 | −43.991 | −5.646 | 1.00 | 50.00 | O |
| ATOM | 3095 | N | PHE | B | 488 | 0.442 | −43.088 | −5.625 | 1.00 | 50.00 | N |
| ATOM | 3096 | CA | PHE | B | 488 | −0.141 | −43.552 | −4.366 | 1.00 | 50.00 | C |
| ATOM | 3097 | C | PHE | B | 488 | −1.422 | −44.373 | −4.488 | 1.00 | 50.00 | C |
| ATOM | 3098 | O | PHE | B | 488 | −1.486 | −45.462 | −3.920 | 1.00 | 50.00 | O |
| ATOM | 3099 | CB | PHE | B | 488 | −0.270 | −42.346 | −3.434 | 1.00 | 50.00 | C |
| ATOM | 3100 | CG | PHE | B | 488 | −0.736 | −42.697 | −2.038 | 1.00 | 50.00 | C |
| ATOM | 3101 | CD1 | PHE | B | 488 | 0.135 | −43.138 | −1.107 | 1.00 | 50.00 | C |
| ATOM | 3102 | CD2 | PHE | B | 488 | −2.060 | −42.709 | −1.775 | 1.00 | 50.00 | C |
| ATOM | 3103 | CE1 | PHE | B | 488 | −0.312 | −43.562 | 0.093 | 1.00 | 50.00 | C |
| ATOM | 3104 | CE2 | PHE | B | 488 | −2.510 | −43.173 | −0.593 | 1.00 | 50.00 | C |
| ATOM | 3105 | CZ | PHE | B | 488 | −1.634 | −43.560 | 0.355 | 1.00 | 50.00 | C |
| ATOM | 3106 | N | ASP | B | 489 | −2.416 | −43.878 | −5.207 | 1.00 | 50.00 | N |
| ATOM | 3107 | CA | ASP | B | 489 | −3.666 | −44.582 | −5.498 | 1.00 | 50.00 | C |
| ATOM | 3108 | C | ASP | B | 489 | −3.514 | −45.881 | −6.283 | 1.00 | 50.00 | C |
| ATOM | 3109 | O | ASP | B | 489 | −4.119 | −46.876 | −5.885 | 1.00 | 50.00 | O |
| ATOM | 3110 | CB | ASP | B | 489 | −4.646 | −43.620 | −6.171 | 1.00 | 50.00 | C |
| ATOM | 3111 | CG | ASP | B | 489 | −5.117 | −42.392 | −5.402 | 1.00 | 50.00 | C |
| ATOM | 3112 | OD1 | ASP | B | 489 | −4.963 | −42.322 | −4.164 | 1.00 | 50.00 | O |
| ATOM | 3113 | OD2 | ASP | B | 489 | −5.653 | −41.467 | −6.050 | 1.00 | 50.00 | O |
| ATOM | 3114 | N | ALA | B | 490 | −2.700 | −45.922 | −7.326 | 1.00 | 50.00 | N |
| ATOM | 3115 | CA | ALA | B | 490 | −2.242 | −47.153 | −7.972 | 1.00 | 50.00 | C |
| ATOM | 3116 | C | ALA | B | 490 | −1.523 | −48.182 | −7.107 | 1.00 | 50.00 | C |
| ATOM | 3117 | O | ALA | B | 490 | −1.824 | −49.368 | −7.237 | 1.00 | 50.00 | O |
| ATOM | 3118 | CB | ALA | B | 490 | −1.379 | −46.770 | −9.173 | 1.00 | 50.00 | C |
| ATOM | 3119 | N | SER | B | 491 | −0.620 | −47.778 | −6.227 | 1.00 | 50.00 | N |
| ATOM | 3120 | CA | SER | B | 491 | −0.007 | −48.633 | −5.210 | 1.00 | 50.00 | C |
| ATOM | 3121 | C | SER | B | 491 | −0.954 | −49.205 | −4.161 | 1.00 | 50.00 | C |
| ATOM | 3122 | O | SER | B | 491 | −0.845 | −50.400 | −3.894 | 1.00 | 50.00 | O |
| ATOM | 3123 | CB | SER | B | 491 | 1.160 | −47.904 | −4.543 | 1.00 | 50.00 | C |
| ATOM | 3124 | OG | SER | B | 491 | 2.201 | −47.654 | −5.477 | 1.00 | 50.00 | O |
| ATOM | 3125 | N | ILE | B | 492 | −1.890 | −48.448 | −3.610 | 1.00 | 50.00 | N |
| ATOM | 3126 | CA | ILE | B | 492 | −3.009 | −48.962 | −2.816 | 1.00 | 50.00 | C |
| ATOM | 3127 | C | ILE | B | 492 | −3.881 | −49.975 | −3.552 | 1.00 | 50.00 | C |
| ATOM | 3128 | O | ILE | B | 492 | −4.206 | −50.999 | −2.953 | 1.00 | 50.00 | O |
| ATOM | 3129 | CB | ILE | B | 492 | −3.812 | −47.836 | −2.155 | 1.00 | 50.00 | C |
| ATOM | 3130 | CG1 | ILE | B | 492 | −2.952 | −47.067 | −1.150 | 1.00 | 50.00 | C |
| ATOM | 3131 | CG2 | ILE | B | 492 | −5.137 | −48.282 | −1.534 | 1.00 | 50.00 | C |
| ATOM | 3132 | CD1 | ILE | B | 492 | −3.694 | −46.162 | −0.170 | 1.00 | 50.00 | C |
| ATOM | 3133 | N | SER | B | 493 | −4.292 | −49.728 | −4.785 | 1.00 | 50.00 | N |
| ATOM | 3134 | CA | SER | B | 493 | −4.955 | −50.726 | −5.624 | 1.00 | 50.00 | C |
| ATOM | 3135 | C | SER | B | 493 | −4.175 | −52.012 | −5.882 | 1.00 | 50.00 | C |
| ATOM | 3136 | O | SER | B | 493 | −4.790 | −53.077 | −5.843 | 1.00 | 50.00 | O |
| ATOM | 3137 | CB | SER | B | 493 | −5.423 | −50.038 | −6.906 | 1.00 | 50.00 | C |
| ATOM | 3138 | OG | SER | B | 493 | −6.320 | −50.874 | −7.624 | 1.00 | 50.00 | O |
| ATOM | 3139 | N | GLN | B | 494 | −2.865 | −51.961 | −6.062 | 1.00 | 50.00 | N |
| ATOM | 3140 | CA | GLN | B | 494 | −1.962 | −53.108 | −5.941 | 1.00 | 50.00 | C |
| ATOM | 3141 | C | GLN | B | 494 | −1.960 | −53.831 | −4.596 | 1.00 | 50.00 | C |
| ATOM | 3142 | O | GLN | B | 494 | −1.949 | −55.061 | −4.620 | 1.00 | 50.00 | O |
| ATOM | 3143 | CB | GLN | B | 494 | −0.564 | −52.669 | −6.377 | 1.00 | 50.00 | C |
| ATOM | 3144 | CG | GLN | B | 494 | 0.446 | −53.790 | −6.622 | 1.00 | 50.00 | C |
| ATOM | 3145 | CD | GLN | B | 494 | 1.866 | −53.342 | −6.940 | 1.00 | 50.00 | C |
| ATOM | 3146 | OE1 | GLN | B | 494 | 2.207 | −52.161 | −6.994 | 1.00 | 50.00 | O |
| ATOM | 3147 | NE2 | GLN | B | 494 | 2.753 | −54.298 | −7.149 | 1.00 | 50.00 | N |
| ATOM | 3148 | N | VAL | B | 495 | −2.004 | −53.159 | −3.455 | 1.00 | 50.00 | N |
| ATOM | 3149 | CA | VAL | B | 495 | −2.258 | −53.753 | −2.140 | 1.00 | 50.00 | C |
| ATOM | 3150 | C | VAL | B | 495 | −3.569 | −54.530 | −2.101 | 1.00 | 50.00 | C |
| ATOM | 3151 | O | VAL | B | 495 | −3.520 | −55.736 | −1.864 | 1.00 | 50.00 | O |
| ATOM | 3152 | CB | VAL | B | 495 | −2.176 | −52.778 | −0.961 | 1.00 | 50.00 | C |
| ATOM | 3153 | CG1 | VAL | B | 495 | −2.364 | −53.406 | 0.421 | 1.00 | 50.00 | C |
| ATOM | 3154 | CG2 | VAL | B | 495 | −0.856 | −52.016 | −0.873 | 1.00 | 50.00 | C |
| ATOM | 3155 | N | ASN | B | 496 | −4.702 | −53.889 | −2.338 | 1.00 | 50.00 | N |
| ATOM | 3156 | CA | ASN | B | 496 | −6.017 | −54.525 | −2.425 | 1.00 | 50.00 | C |
| ATOM | 3157 | C | ASN | B | 496 | −6.144 | −55.690 | −3.402 | 1.00 | 50.00 | C |
| ATOM | 3158 | O | ASN | B | 496 | −6.775 | −56.682 | −3.042 | 1.00 | 50.00 | O |
| ATOM | 3159 | CB | ASN | B | 496 | −7.083 | −53.458 | −2.676 | 1.00 | 50.00 | C |
| ATOM | 3160 | CG | ASN | B | 496 | −7.318 | −52.457 | −1.551 | 1.00 | 50.00 | C |
| ATOM | 3161 | OD1 | ASN | B | 496 | −7.607 | −52.823 | −0.413 | 1.00 | 50.00 | O |
| ATOM | 3162 | ND2 | ASN | B | 496 | −7.210 | −51.170 | −1.826 | 1.00 | 50.00 | N |
| ATOM | 3163 | N | GLU | B | 497 | −5.513 | −55.640 | −4.565 | 1.00 | 50.00 | N |
| ATOM | 3164 | CA | GLU | B | 497 | −5.273 | −56.792 | −5.435 | 1.00 | 50.00 | C |
| ATOM | 3165 | C | GLU | B | 497 | −4.526 | −57.951 | −4.782 | 1.00 | 50.00 | C |
| ATOM | 3166 | O | GLU | B | 497 | −5.058 | −59.059 | −4.816 | 1.00 | 50.00 | O |
| ATOM | 3167 | CB | GLU | B | 497 | −4.612 | −56.315 | −6.730 | 1.00 | 50.00 | C |
| ATOM | 3168 | CG | GLU | B | 497 | −4.625 | −57.328 | −7.874 | 1.00 | 50.00 | C |
| ATOM | 3169 | CD | GLU | B | 497 | −3.931 | −56.843 | −9.138 | 1.00 | 50.00 | C |
| ATOM | 3170 | OE1 | GLU | B | 497 | −4.381 | −55.840 | −9.732 | 1.00 | 50.00 | O |
| ATOM | 3171 | OE2 | GLU | B | 497 | −2.934 | −57.470 | −9.557 | 1.00 | 50.00 | O |
| ATOM | 3172 | N | LYS | B | 498 | −3.370 | −57.744 | −4.171 | 1.00 | 50.00 | N |
| ATOM | 3173 | CA | LYS | B | 498 | −2.675 | −58.743 | −3.356 | 1.00 | 50.00 | C |
| ATOM | 3174 | C | LYS | B | 498 | −3.415 | −59.323 | −2.155 | 1.00 | 50.00 | C |
| ATOM | 3175 | O | LYS | B | 498 | −3.251 | −60.514 | −1.891 | 1.00 | 50.00 | O |
| ATOM | 3176 | CB | LYS | B | 498 | −1.291 | −58.237 | −2.945 | 1.00 | 50.00 | C |
| ATOM | 3177 | CG | LYS | B | 498 | −0.248 | −58.067 | −4.050 | 1.00 | 50.00 | C |
| ATOM | 3178 | CD | LYS | B | 498 | 0.212 | −59.378 | −4.683 | 1.00 | 50.00 | C |
| ATOM | 3179 | CE | LYS | B | 498 | 1.074 | −59.194 | −5.930 | 1.00 | 50.00 | C |
| ATOM | 3180 | NZ | LYS | B | 498 | 1.354 | −60.496 | −6.553 | 1.00 | 50.00 | N |
| ATOM | 3181 | N | ILE | B | 499 | −4.240 | −58.559 | −1.456 | 1.00 | 50.00 | N |
| ATOM | 3182 | CA | ILE | B | 499 | −5.197 | −59.049 | −0.460 | 1.00 | 50.00 | C |
| ATOM | 3183 | C | ILE | B | 499 | −6.262 | −59.952 | −1.077 | 1.00 | 50.00 | C |
| ATOM | 3184 | O | ILE | B | 499 | −6.483 | −61.037 | −0.542 | 1.00 | 50.00 | O |
| ATOM | 3185 | CB | ILE | B | 499 | −5.771 | −57.930 | 0.415 | 1.00 | 50.00 | C |
| ATOM | 3186 | CG1 | ILE | B | 499 | −4.724 | −57.029 | 1.076 | 1.00 | 50.00 | C |
| ATOM | 3187 | CG2 | ILE | B | 499 | −6.706 | −58.469 | 1.499 | 1.00 | 50.00 | C |
| ATOM | 3188 | CD1 | ILE | B | 499 | −5.229 | −55.819 | 1.858 | 1.00 | 50.00 | C |
| ATOM | 3189 | N | ASN | B | 500 | −6.930 | −59.569 | −2.154 | 1.00 | 50.00 | N |
| ATOM | 3190 | CA | ASN | B | 500 | −7.867 | −60.423 | −2.888 | 1.00 | 50.00 | C |
| ATOM | 3191 | C | ASN | B | 500 | −7.288 | −61.694 | −3.502 | 1.00 | 50.00 | C |
| ATOM | 3192 | O | ASN | B | 500 | −7.941 | −62.731 | −3.399 | 1.00 | 50.00 | O |
| ATOM | 3193 | CB | ASN | B | 500 | −8.643 | −59.595 | −3.913 | 1.00 | 50.00 | C |
| ATOM | 3194 | CG | ASN | B | 500 | −9.638 | −58.582 | −3.359 | 1.00 | 50.00 | C |
| ATOM | 3195 | OD1 | ASN | B | 500 | −10.414 | −58.868 | −2.449 | 1.00 | 50.00 | O |
| ATOM | 3196 | ND2 | ASN | B | 500 | −9.670 | −57.377 | −3.898 | 1.00 | 50.00 | N |
| ATOM | 3197 | N | GLN | B | 501 | −6.091 | −61.677 | −4.070 | 1.00 | 50.00 | N |
| ATOM | 3198 | CA | GLN | B | 501 | −5.284 | −62.864 | −4.361 | 1.00 | 50.00 | C |
| ATOM | 3199 | C | GLN | B | 501 | −5.015 | −63.793 | −3.180 | 1.00 | 50.00 | C |
| ATOM | 3200 | O | GLN | B | 501 | −5.223 | −64.996 | −3.329 | 1.00 | 50.00 | O |
| ATOM | 3201 | CB | GLN | B | 501 | −3.935 | −62.496 | −4.981 | 1.00 | 50.00 | C |
| ATOM | 3202 | CG | GLN | B | 501 | −3.904 | −61.830 | −6.356 | 1.00 | 50.00 | C |
| ATOM | 3203 | CD | GLN | B | 501 | −2.486 | −61.481 | −6.787 | 1.00 | 50.00 | C |
| ATOM | 3204 | OE1 | GLN | B | 501 | −1.570 | −62.299 | −6.725 | 1.00 | 50.00 | O |
| ATOM | 3205 | NE2 | GLN | B | 501 | −2.243 | −60.261 | −7.230 | 1.00 | 50.00 | N |
| ATOM | 3206 | N | SER | B | 502 | −4.584 | −63.286 | −2.035 | 1.00 | 50.00 | N |
| ATOM | 3207 | CA | SER | B | 502 | −4.494 | −64.024 | −0.773 | 1.00 | 50.00 | C |
| ATOM | 3208 | C | SER | B | 502 | −5.768 | −64.745 | −0.346 | 1.00 | 50.00 | C |
| ATOM | 3209 | O | SER | B | 502 | −5.717 | −65.957 | −0.146 | 1.00 | 50.00 | O |
| ATOM | 3210 | CB | SER | B | 502 | −3.982 | −63.084 | 0.318 | 1.00 | 50.00 | C |
| ATOM | 3211 | OG | SER | B | 502 | −3.728 | −63.771 | 1.534 | 1.00 | 50.00 | O |
| ATOM | 3212 | N | LEU | B | 503 | −6.896 | −64.056 | −0.274 | 1.00 | 50.00 | N |
| ATOM | 3213 | CA | LEU | B | 503 | −8.220 | −64.648 | −0.076 | 1.00 | 50.00 | C |
| ATOM | 3214 | C | LEU | B | 503 | −8.675 | −65.678 | −1.106 | 1.00 | 50.00 | C |
| ATOM | 3215 | O | LEU | B | 503 | −9.323 | −66.642 | −0.701 | 1.00 | 50.00 | O |
| ATOM | 3216 | CB | LEU | B | 503 | −9.262 | −63.542 | 0.106 | 1.00 | 50.00 | C |
| ATOM | 3217 | CG | LEU | B | 503 | −9.102 | −62.565 | 1.273 | 1.00 | 50.00 | C |
| ATOM | 3218 | CD1 | LEU | B | 503 | −10.142 | −61.448 | 1.202 | 1.00 | 50.00 | C |
| ATOM | 3219 | CD2 | LEU | B | 503 | −9.146 | −63.234 | 2.646 | 1.00 | 50.00 | C |
| ATOM | 3220 | N | ALA | B | 504 | −8.343 | −65.542 | −2.380 | 1.00 | 50.00 | N |
| ATOM | 3221 | CA | ALA | B | 504 | −8.513 | −66.578 | −3.401 | 1.00 | 50.00 | C |
| ATOM | 3222 | C | ALA | B | 504 | −7.663 | −67.836 | −3.253 | 1.00 | 50.00 | C |
| ATOM | 3223 | O | ALA | B | 504 | −8.229 | −68.924 | −3.346 | 1.00 | 50.00 | O |
| ATOM | 3224 | CB | ALA | B | 504 | −8.312 | −65.946 | −4.777 | 1.00 | 50.00 | C |
| ATOM | 3225 | N | PHE | B | 505 | −6.366 | −67.746 | −3.001 | 1.00 | 50.00 | N |
| ATOM | 3226 | CA | PHE | B | 505 | −5.530 | −68.879 | −2.598 | 1.00 | 50.00 | C |
| ATOM | 3227 | C | PHE | B | 505 | −5.959 | −69.635 | −1.346 | 1.00 | 50.00 | C |
| ATOM | 3228 | O | PHE | B | 505 | −5.895 | −70.864 | −1.335 | 1.00 | 50.00 | O |
| ATOM | 3229 | CB | PHE | B | 505 | −4.056 | −68.479 | −2.493 | 1.00 | 50.00 | C |
| ATOM | 3230 | CG | PHE | B | 505 | −3.301 | −68.231 | −3.782 | 1.00 | 50.00 | C |
| ATOM | 3231 | CD1 | PHE | B | 505 | −2.598 | −67.092 | −3.952 | 1.00 | 50.00 | C |
| ATOM | 3232 | CD2 | PHE | B | 505 | −3.293 | −69.162 | −4.759 | 1.00 | 50.00 | C |
| ATOM | 3233 | CE1 | PHE | B | 505 | −1.923 | −66.869 | −5.098 | 1.00 | 50.00 | C |
| ATOM | 3234 | CE2 | PHE | B | 505 | −2.632 | −68.940 | −5.913 | 1.00 | 50.00 | C |
| ATOM | 3235 | CZ | PHE | B | 505 | −1.947 | −67.791 | −6.081 | 1.00 | 50.00 | C |
| ATOM | 3236 | N | ILE | B | 506 | −6.433 | −68.938 | −0.326 | 1.00 | 50.00 | N |
| ATOM | 3237 | CA | ILE | B | 506 | −7.150 | −69.516 | 0.810 | 1.00 | 50.00 | C |
| ATOM | 3238 | C | ILE | B | 506 | −8.464 | −70.199 | 0.449 | 1.00 | 50.00 | C |
| ATOM | 3239 | O | ILE | B | 506 | −8.667 | −71.324 | 0.904 | 1.00 | 50.00 | O |
| ATOM | 3240 | CB | ILE | B | 506 | −7.276 | −68.476 | 1.927 | 1.00 | 50.00 | C |
| ATOM | 3241 | CG1 | ILE | B | 506 | −5.947 | −67.984 | 2.503 | 1.00 | 50.00 | C |
| ATOM | 3242 | CG2 | ILE | B | 506 | −8.160 | −68.977 | 3.070 | 1.00 | 50.00 | C |
| ATOM | 3243 | CD1 | ILE | B | 506 | −5.087 | −69.106 | 3.080 | 1.00 | 50.00 | C |
| ATOM | 3244 | N | ARG | B | 507 | −9.353 | −69.567 | −0.300 | 1.00 | 50.00 | N |
| ATOM | 3245 | CA | ARG | B | 507 | −10.562 | −70.196 | −0.835 | 1.00 | 50.00 | C |
| ATOM | 3246 | C | ARG | B | 507 | −10.356 | −71.536 | −1.535 | 1.00 | 50.00 | C |
| ATOM | 3247 | O | ARG | B | 507 | −11.114 | −72.466 | −1.262 | 1.00 | 50.00 | O |
| ATOM | 3248 | CB | ARG | B | 507 | −11.296 | −69.173 | −1.704 | 1.00 | 50.00 | C |
| ATOM | 3249 | CG | ARG | B | 507 | −12.751 | −69.542 | −1.984 | 1.00 | 50.00 | C |
| ATOM | 3250 | CD | ARG | B | 507 | −13.449 | −68.694 | −3.047 | 1.00 | 50.00 | C |
| ATOM | 3251 | NE | ARG | B | 507 | −14.905 | −68.842 | −3.054 | 1.00 | 50.00 | N |
| ATOM | 3252 | CZ | ARG | B | 507 | −15.574 | −69.955 | −3.309 | 1.00 | 50.00 | C |
| ATOM | 3253 | NH1 | ARG | B | 507 | −15.021 | −71.095 | −3.682 | 1.00 | 50.00 | N |
| ATOM | 3254 | NH2 | ARG | B | 507 | −16.886 | −69.934 | −3.167 | 1.00 | 50.00 | N |
| ATOM | 3255 | N | LYS | B | 508 | −9.331 | −71.671 | −2.360 | 1.00 | 50.00 | N |
| ATOM | 3256 | CA | LYS | B | 508 | −8.894 | −72.938 | −2.947 | 1.00 | 50.00 | C |
| ATOM | 3257 | C | LYS | B | 508 | −8.327 | −73.974 | −1.983 | 1.00 | 50.00 | C |
| ATOM | 3258 | O | LYS | B | 508 | −8.672 | −75.149 | −2.108 | 1.00 | 50.00 | O |
| ATOM | 3259 | CB | LYS | B | 508 | −7.893 | −72.675 | −4.071 | 1.00 | 50.00 | C |
| ATOM | 3260 | CG | LYS | B | 508 | −8.455 | −71.892 | −5.256 | 1.00 | 50.00 | C |
| ATOM | 3261 | CD | LYS | B | 508 | −7.512 | −71.887 | −6.457 | 1.00 | 50.00 | C |
| ATOM | 3262 | CE | LYS | B | 508 | −8.132 | −71.171 | −7.654 | 1.00 | 50.00 | C |
| ATOM | 3263 | NZ | LYS | B | 508 | −7.245 | −71.258 | −8.823 | 1.00 | 50.00 | N |
| ATOM | 3264 | N | SER | B | 509 | −7.491 | −73.593 | −1.031 | 1.00 | 50.00 | N |
| ATOM | 3265 | CA | SER | B | 509 | −7.060 | −74.455 | 0.071 | 1.00 | 50.00 | C |
| ATOM | 3266 | C | SER | B | 509 | −8.177 | −75.020 | 0.941 | 1.00 | 50.00 | C |
| ATOM | 3267 | O | SER | B | 509 | −8.220 | −76.236 | 1.123 | 1.00 | 50.00 | O |
| ATOM | 3268 | CB | SER | B | 509 | −6.033 | −73.661 | 0.876 | 1.00 | 50.00 | C |
| ATOM | 3269 | OG | SER | B | 509 | −5.229 | −74.524 | 1.665 | 1.00 | 50.00 | O |
| ATOM | 3270 | N | ASP | B | 510 | −9.110 | −74.200 | 1.399 | 1.00 | 50.00 | N |
| ATOM | 3271 | CA | ASP | B | 510 | −10.368 | −74.616 | 2.020 | 1.00 | 50.00 | C |
| ATOM | 3272 | C | ASP | B | 510 | −11.229 | −75.563 | 1.190 | 1.00 | 50.00 | C |
| ATOM | 3273 | O | ASP | B | 510 | −11.602 | −76.608 | 1.722 | 1.00 | 50.00 | O |
| ATOM | 3274 | CB | ASP | B | 510 | −11.170 | −73.397 | 2.481 | 1.00 | 50.00 | C |
| ATOM | 3275 | CG | ASP | B | 510 | −10.533 | −72.465 | 3.502 | 1.00 | 50.00 | C |
| ATOM | 3276 | OD1 | ASP | B | 510 | −9.958 | −72.947 | 4.502 | 1.00 | 50.00 | O |
| ATOM | 3277 | OD2 | ASP | B | 510 | −10.601 | −71.231 | 3.317 | 1.00 | 50.00 | O |
| ATOM | 3278 | N | GLU | B | 511 | −11.496 | −75.286 | −0.078 | 1.00 | 50.00 | N |
| ATOM | 3279 | CA | GLU | B | 511 | −12.096 | −76.214 | −1.038 | 1.00 | 50.00 | C |
| ATOM | 3280 | C | GLU | B | 511 | −11.505 | −77.620 | −1.081 | 1.00 | 50.00 | C |
| ATOM | 3281 | O | GLU | B | 511 | −12.243 | −78.577 | −0.848 | 1.00 | 50.00 | O |
| ATOM | 3282 | CB | GLU | B | 511 | −12.080 | −75.614 | −2.446 | 1.00 | 50.00 | C |
| ATOM | 3283 | CG | GLU | B | 511 | −13.078 | −74.510 | −2.788 | 1.00 | 50.00 | C |
| ATOM | 3284 | CD | GLU | B | 511 | −12.769 | −73.886 | −4.142 | 1.00 | 50.00 | C |
| ATOM | 3285 | OE1 | GLU | B | 511 | −12.878 | −74.585 | −5.172 | 1.00 | 50.00 | O |
| ATOM | 3286 | OE2 | GLU | B | 511 | −12.393 | −72.694 | −4.190 | 1.00 | 50.00 | O |
| ATOM | 3287 | N | LEU | B | 512 | −10.213 | −77.758 | −1.335 | 1.00 | 50.00 | N |
| ATOM | 3288 | CA | LEU | B | 512 | −9.472 | −79.020 | −1.279 | 1.00 | 50.00 | C |
| ATOM | 3289 | C | LEU | B | 512 | −9.572 | −79.818 | 0.016 | 1.00 | 50.00 | C |
| ATOM | 3290 | O | LEU | B | 512 | −9.803 | −81.026 | −0.037 | 1.00 | 50.00 | O |
| ATOM | 3291 | CB | LEU | B | 512 | −7.993 | −78.786 | −1.591 | 1.00 | 50.00 | C |
| ATOM | 3292 | CG | LEU | B | 512 | −7.606 | −78.306 | −2.990 | 1.00 | 50.00 | C |
| ATOM | 3293 | CD1 | LEU | B | 512 | −6.117 | −77.967 | −3.030 | 1.00 | 50.00 | C |
| ATOM | 3294 | CD2 | LEU | B | 512 | −7.959 | −79.315 | −4.081 | 1.00 | 50.00 | C |
| ATOM | 3295 | N | LEU | B | 513 | −9.436 | −79.164 | 1.158 | 1.00 | 50.00 | N |
| ATOM | 3296 | CA | LEU | B | 513 | −9.731 | −79.722 | 2.477 | 1.00 | 50.00 | C |
| ATOM | 3297 | C | LEU | B | 513 | −11.142 | −80.294 | 2.575 | 1.00 | 50.00 | C |
| ATOM | 3298 | O | LEU | B | 513 | −11.278 | −81.511 | 2.700 | 1.00 | 50.00 | O |
| ATOM | 3299 | CB | LEU | B | 513 | −9.375 | −78.691 | 3.549 | 1.00 | 50.00 | C |
| ATOM | 3300 | CG | LEU | B | 513 | −7.925 | −78.209 | 3.645 | 1.00 | 50.00 | C |
| ATOM | 3301 | CD1 | LEU | B | 513 | −7.861 | −76.973 | 4.539 | 1.00 | 50.00 | C |
| ATOM | 3302 | CD2 | LEU | B | 513 | −6.961 | −79.283 | 4.144 | 1.00 | 50.00 | C |
| ATOM | 3303 | N | HIS | B | 514 | −12.201 | −79.529 | 2.365 | 1.00 | 50.00 | N |
| ATOM | 3304 | CA | HIS | B | 514 | −13.573 | −80.040 | 2.312 | 1.00 | 50.00 | C |
| ATOM | 3305 | C | HIS | B | 514 | −13.923 | −81.148 | 1.321 | 1.00 | 50.00 | C |
| ATOM | 3306 | O | HIS | B | 514 | −14.935 | −81.814 | 1.541 | 1.00 | 50.00 | O |
| ATOM | 3307 | CB | HIS | B | 514 | −14.552 | −78.867 | 2.224 | 1.00 | 50.00 | C |
| ATOM | 3308 | CG | HIS | B | 514 | −14.529 | −77.858 | 3.352 | 1.00 | 50.00 | C |
| ATOM | 3309 | ND1 | HIS | B | 514 | −14.621 | −78.186 | 4.691 | 1.00 | 50.00 | N |
| ATOM | 3310 | CD2 | HIS | B | 514 | −14.293 | −76.496 | 3.187 | 1.00 | 50.00 | C |
| ATOM | 3311 | CE1 | HIS | B | 514 | −14.391 | −76.941 | 5.238 | 1.00 | 50.00 | C |
| ATOM | 3312 | NE2 | HIS | B | 514 | −14.185 | −75.860 | 4.407 | 1.00 | 50.00 | N |
| ATOM | 3313 | N | ASN | B | 515 | −13.124 | −81.431 | 0.304 | 1.00 | 50.00 | N |
| ATOM | 3314 | CA | ASN | B | 515 | −13.266 | −82.586 | −0.583 | 1.00 | 50.00 | C |
| ATOM | 3315 | C | ASN | B | 515 | −12.926 | −83.880 | 0.148 | 1.00 | 50.00 | C |
| ATOM | 3316 | O | ASN | B | 515 | −11.834 | −84.445 | 0.110 | 1.00 | 50.00 | O |
| ATOM | 3317 | CB | ASN | B | 515 | −12.420 | −82.345 | −1.835 | 1.00 | 50.00 | C |
| ATOM | 3318 | CG | ASN | B | 515 | −12.620 | −83.331 | −2.978 | 1.00 | 50.00 | C |
| ATOM | 3319 | OD1 | ASN | B | 515 | −12.112 | −84.451 | −2.953 | 1.00 | 50.00 | O |
| ATOM | 3320 | ND2 | ASN | B | 515 | −13.349 | −82.958 | −4.014 | 1.00 | 50.00 | N |
| ATOM | 3321 | N | THR | C | 29 | 5.374 | 50.147 | 36.915 | 1.00 | 50.00 | N |
| ATOM | 3322 | CA | THR | C | 29 | 4.465 | 51.294 | 37.078 | 1.00 | 50.00 | C |
| ATOM | 3323 | C | THR | C | 29 | 3.607 | 51.223 | 38.335 | 1.00 | 50.00 | C |
| ATOM | 3324 | O | THR | C | 29 | 3.425 | 52.280 | 38.937 | 1.00 | 50.00 | O |
| ATOM | 3325 | CB | THR | C | 29 | 3.665 | 51.574 | 35.804 | 1.00 | 50.00 | C |
| ATOM | 3326 | OG1 | THR | C | 29 | 4.561 | 51.985 | 34.781 | 1.00 | 50.00 | O |
| ATOM | 3327 | CG2 | THR | C | 29 | 2.593 | 52.659 | 35.900 | 1.00 | 50.00 | C |
| ATOM | 3328 | N | GLU | C | 30 | 3.013 | 50.090 | 38.679 | 1.00 | 50.00 | N |
| ATOM | 3329 | CA | GLU | C | 30 | 1.968 | 49.978 | 39.698 | 1.00 | 50.00 | C |
| ATOM | 3330 | C | GLU | C | 30 | 0.597 | 50.348 | 39.139 | 1.00 | 50.00 | C |
| ATOM | 3331 | O | GLU | C | 30 | 0.397 | 50.586 | 37.948 | 1.00 | 50.00 | O |
| ATOM | 3332 | CB | GLU | C | 30 | 2.325 | 50.569 | 41.065 | 1.00 | 50.00 | C |
| ATOM | 3333 | CG | GLU | C | 30 | 3.566 | 50.003 | 41.755 | 1.00 | 50.00 | C |
| ATOM | 3334 | CD | GLU | C | 30 | 3.292 | 48.664 | 42.423 | 1.00 | 50.00 | C |
| ATOM | 3335 | OE1 | GLU | C | 30 | 3.454 | 47.613 | 41.766 | 1.00 | 50.00 | O |
| ATOM | 3336 | OE2 | GLU | C | 30 | 2.886 | 48.656 | 43.605 | 1.00 | 50.00 | O |
| ATOM | 3337 | N | GLU | C | 31 | −0.382 | 50.309 | 40.027 | 1.00 | 50.00 | N |
| ATOM | 3338 | CA | GLU | C | 31 | −1.798 | 50.524 | 39.735 | 1.00 | 50.00 | C |
| ATOM | 3339 | C | GLU | C | 31 | −2.465 | 51.528 | 40.668 | 1.00 | 50.00 | C |
| ATOM | 3340 | O | GLU | C | 31 | −3.683 | 51.474 | 40.838 | 1.00 | 50.00 | O |
| ATOM | 3341 | CB | GLU | C | 31 | −2.474 | 49.158 | 39.857 | 1.00 | 50.00 | C |
| ATOM | 3342 | CG | GLU | C | 31 | −2.032 | 48.025 | 38.931 | 1.00 | 50.00 | C |
| ATOM | 3343 | CD | GLU | C | 31 | −2.191 | 48.200 | 37.429 | 1.00 | 50.00 | C |
| ATOM | 3344 | OE1 | GLU | C | 31 | −1.888 | 49.278 | 36.873 | 1.00 | 50.00 | O |
| ATOM | 3345 | OE2 | GLU | C | 31 | −2.631 | 47.227 | 36.779 | 1.00 | 50.00 | O |
| ATOM | 3346 | N | PHE | C | 32 | −1.760 | 52.522 | 41.184 | 1.00 | 50.00 | N |
| ATOM | 3347 | CA | PHE | C | 32 | −2.409 | 53.754 | 41.631 | 1.00 | 50.00 | C |
| ATOM | 3348 | C | PHE | C | 32 | −3.000 | 54.541 | 40.465 | 1.00 | 50.00 | C |
| ATOM | 3349 | O | PHE | C | 32 | −4.132 | 55.012 | 40.576 | 1.00 | 50.00 | O |
| ATOM | 3350 | CB | PHE | C | 32 | −1.557 | 54.527 | 42.639 | 1.00 | 50.00 | C |
| ATOM | 3351 | CG | PHE | C | 32 | −2.416 | 55.337 | 43.590 | 1.00 | 50.00 | C |
| ATOM | 3352 | CD1 | PHE | C | 32 | −2.560 | 55.106 | 44.911 | 1.00 | 50.00 | C |
| ATOM | 3353 | CD2 | PHE | C | 32 | −3.167 | 56.300 | 43.022 | 1.00 | 50.00 | C |
| ATOM | 3354 | CE1 | PHE | C | 32 | −3.366 | 55.889 | 45.658 | 1.00 | 50.00 | C |
| ATOM | 3355 | CE2 | PHE | C | 32 | −4.037 | 57.037 | 43.741 | 1.00 | 50.00 | C |
| ATOM | 3356 | CZ | PHE | C | 32 | −4.078 | 56.876 | 45.077 | 1.00 | 50.00 | C |
| ATOM | 3357 | N | TYR | C | 33 | −2.335 | 54.621 | 39.324 | 1.00 | 50.00 | N |
| ATOM | 3358 | CA | TYR | C | 33 | −2.947 | 55.016 | 38.056 | 1.00 | 50.00 | C |
| ATOM | 3359 | C | TYR | C | 33 | −4.231 | 54.371 | 37.540 | 1.00 | 50.00 | C |
| ATOM | 3360 | O | TYR | C | 33 | −4.859 | 54.906 | 36.626 | 1.00 | 50.00 | O |
| ATOM | 3361 | CB | TYR | C | 33 | −1.782 | 55.026 | 37.063 | 1.00 | 50.00 | C |
| ATOM | 3362 | CG | TYR | C | 33 | −2.076 | 55.695 | 35.739 | 1.00 | 50.00 | C |
| ATOM | 3363 | CD1 | TYR | C | 33 | −2.805 | 56.828 | 35.761 | 1.00 | 50.00 | C |
| ATOM | 3364 | CD2 | TYR | C | 33 | −1.802 | 55.110 | 34.554 | 1.00 | 50.00 | C |
| ATOM | 3365 | CE1 | TYR | C | 33 | −3.456 | 57.233 | 34.654 | 1.00 | 50.00 | C |
| ATOM | 3366 | CE2 | TYR | C | 33 | −2.253 | 55.668 | 33.413 | 1.00 | 50.00 | C |
| ATOM | 3367 | CZ | TYR | C | 33 | −3.145 | 56.681 | 33.467 | 1.00 | 50.00 | C |
| ATOM | 3368 | OH | TYR | C | 33 | −3.642 | 57.312 | 32.357 | 1.00 | 50.00 | O |
| ATOM | 3369 | N | GLN | C | 34 | −4.711 | 53.298 | 38.144 | 1.00 | 50.00 | N |
| ATOM | 3370 | CA | GLN | C | 34 | −6.089 | 52.825 | 38.002 | 1.00 | 50.00 | C |
| ATOM | 3371 | C | GLN | C | 34 | −7.193 | 53.768 | 38.468 | 1.00 | 50.00 | C |
| ATOM | 3372 | O | GLN | C | 34 | −8.161 | 53.947 | 37.731 | 1.00 | 50.00 | O |
| ATOM | 3373 | CB | GLN | C | 34 | −6.229 | 51.485 | 38.724 | 1.00 | 50.00 | C |
| ATOM | 3374 | CG | GLN | C | 34 | −5.399 | 50.334 | 38.158 | 1.00 | 50.00 | C |
| ATOM | 3375 | CD | GLN | C | 34 | −5.827 | 49.802 | 36.800 | 1.00 | 50.00 | C |
| ATOM | 3376 | OE1 | GLN | C | 34 | −6.895 | 50.079 | 36.257 | 1.00 | 50.00 | O |
| ATOM | 3377 | NE2 | GLN | C | 34 | −4.948 | 49.002 | 36.226 | 1.00 | 50.00 | N |
| ATOM | 3378 | N | SER | C | 35 | −7.047 | 54.367 | 39.640 | 1.00 | 50.00 | N |
| ATOM | 3379 | CA | SER | C | 35 | −7.841 | 55.506 | 40.103 | 1.00 | 50.00 | C |
| ATOM | 3380 | C | SER | C | 35 | −7.796 | 56.809 | 39.314 | 1.00 | 50.00 | C |
| ATOM | 3381 | O | SER | C | 35 | −8.667 | 57.658 | 39.501 | 1.00 | 50.00 | O |
| ATOM | 3382 | CB | SER | C | 35 | −7.369 | 55.904 | 41.501 | 1.00 | 50.00 | C |
| ATOM | 3383 | OG | SER | C | 35 | −7.595 | 54.879 | 42.456 | 1.00 | 50.00 | O |
| ATOM | 3384 | N | THR | C | 36 | −6.782 | 57.009 | 38.488 | 1.00 | 50.00 | N |
| ATOM | 3385 | CA | THR | C | 36 | −6.783 | 58.040 | 37.453 | 1.00 | 50.00 | C |
| ATOM | 3386 | C | THR | C | 36 | −7.305 | 57.557 | 36.102 | 1.00 | 50.00 | C |
| ATOM | 3387 | O | THR | C | 36 | −7.525 | 58.428 | 35.261 | 1.00 | 50.00 | O |
| ATOM | 3388 | CB | THR | C | 36 | −5.363 | 58.607 | 37.473 | 1.00 | 50.00 | C |
| ATOM | 3389 | OG1 | THR | C | 36 | −4.937 | 59.135 | 38.720 | 1.00 | 50.00 | O |
| ATOM | 3390 | CG2 | THR | C | 36 | −5.126 | 59.744 | 36.485 | 1.00 | 50.00 | C |
| ATOM | 3391 | N | CYS | C | 37 | −7.621 | 56.293 | 35.862 | 1.00 | 50.00 | N |
| ATOM | 3392 | CA | CYS | C | 37 | −8.462 | 55.783 | 34.777 | 1.00 | 50.00 | C |
| ATOM | 3393 | C | CYS | C | 37 | −7.800 | 55.074 | 33.602 | 1.00 | 50.00 | C |
| ATOM | 3394 | O | CYS | C | 37 | −8.357 | 54.968 | 32.510 | 1.00 | 50.00 | O |
| ATOM | 3395 | CB | CYS | C | 37 | −9.562 | 56.741 | 34.328 | 1.00 | 50.00 | C |
| ATOM | 3396 | SG | CYS | C | 37 | −10.238 | 57.636 | 35.738 | 1.00 | 50.00 | S |
| ATOM | 3397 | N | SER | C | 38 | −6.644 | 54.490 | 33.864 | 1.00 | 50.00 | N |
| ATOM | 3398 | CA | SER | C | 38 | −5.967 | 53.569 | 32.955 | 1.00 | 50.00 | C |
| ATOM | 3399 | C | SER | C | 38 | −5.651 | 52.252 | 33.645 | 1.00 | 50.00 | C |
| ATOM | 3400 | O | SER | C | 38 | −5.226 | 52.123 | 34.792 | 1.00 | 50.00 | O |
| ATOM | 3401 | CB | SER | C | 38 | −4.592 | 54.091 | 32.558 | 1.00 | 50.00 | C |
| ATOM | 3402 | OG | SER | C | 38 | −4.689 | 55.074 | 31.539 | 1.00 | 50.00 | O |
| ATOM | 3403 | N | ALA | C | 39 | −5.745 | 51.311 | 32.726 | 1.00 | 50.00 | N |
| ATOM | 3404 | CA | ALA | C | 39 | −5.573 | 49.885 | 32.971 | 1.00 | 50.00 | C |
| ATOM | 3405 | C | ALA | C | 39 | −4.253 | 49.479 | 32.332 | 1.00 | 50.00 | C |
| ATOM | 3406 | O | ALA | C | 39 | −4.054 | 49.646 | 31.129 | 1.00 | 50.00 | O |
| ATOM | 3407 | CB | ALA | C | 39 | −6.882 | 49.295 | 32.451 | 1.00 | 50.00 | C |
| ATOM | 3408 | N | VAL | C | 40 | −3.360 | 48.922 | 33.134 | 1.00 | 50.00 | N |
| ATOM | 3409 | CA | VAL | C | 40 | −2.203 | 48.178 | 32.633 | 1.00 | 50.00 | C |
| ATOM | 3410 | C | VAL | C | 40 | −2.780 | 46.849 | 32.161 | 1.00 | 50.00 | C |
| ATOM | 3411 | O | VAL | C | 40 | −3.113 | 45.978 | 32.965 | 1.00 | 50.00 | O |
| ATOM | 3412 | CB | VAL | C | 40 | −1.062 | 48.053 | 33.647 | 1.00 | 50.00 | C |
| ATOM | 3413 | CG1 | VAL | C | 40 | 0.020 | 47.037 | 33.281 | 1.00 | 50.00 | C |
| ATOM | 3414 | CG2 | VAL | C | 40 | −0.369 | 49.387 | 33.922 | 1.00 | 50.00 | C |
| ATOM | 3415 | N | SER | C | 41 | −2.881 | 46.706 | 30.850 | 1.00 | 50.00 | N |
| ATOM | 3416 | CA | SER | C | 41 | −3.150 | 45.414 | 30.220 | 1.00 | 50.00 | C |
| ATOM | 3417 | C | SER | C | 41 | −2.022 | 44.421 | 30.471 | 1.00 | 50.00 | C |
| ATOM | 3418 | O | SER | C | 41 | −2.322 | 43.336 | 30.968 | 1.00 | 50.00 | O |
| ATOM | 3419 | CB | SER | C | 41 | −3.461 | 45.561 | 28.731 | 1.00 | 50.00 | C |
| ATOM | 3420 | OG | SER | C | 41 | −4.590 | 46.385 | 28.482 | 1.00 | 50.00 | O |
| ATOM | 3421 | N | LYS | C | 42 | −0.779 | 44.742 | 30.145 | 1.00 | 50.00 | N |
| ATOM | 3422 | CA | LYS | C | 42 | 0.321 | 43.778 | 30.135 | 1.00 | 50.00 | C |
| ATOM | 3423 | C | LYS | C | 42 | 1.650 | 44.453 | 30.447 | 1.00 | 50.00 | C |
| ATOM | 3424 | O | LYS | C | 42 | 1.943 | 45.551 | 29.976 | 1.00 | 50.00 | O |
| ATOM | 3425 | CB | LYS | C | 42 | 0.399 | 43.024 | 28.806 | 1.00 | 50.00 | C |
| ATOM | 3426 | CG | LYS | C | 42 | −0.787 | 42.081 | 28.610 | 1.00 | 50.00 | C |
| ATOM | 3427 | CD | LYS | C | 42 | −0.752 | 41.170 | 27.386 | 1.00 | 50.00 | C |
| ATOM | 3428 | CE | LYS | C | 42 | −2.047 | 40.362 | 27.341 | 1.00 | 50.00 | C |
| ATOM | 3429 | NZ | LYS | C | 42 | −2.059 | 39.467 | 26.176 | 1.00 | 50.00 | N |
| ATOM | 3430 | N | GLY | C | 43 | 2.478 | 43.754 | 31.205 | 1.00 | 50.00 | N |
| ATOM | 3431 | CA | GLY | C | 43 | 3.895 | 44.076 | 31.359 | 1.00 | 50.00 | C |
| ATOM | 3432 | C | GLY | C | 43 | 4.723 | 42.947 | 30.766 | 1.00 | 50.00 | C |
| ATOM | 3433 | O | GLY | C | 43 | 4.465 | 41.756 | 30.936 | 1.00 | 50.00 | O |
| ATOM | 3434 | N | TYR | C | 44 | 5.737 | 43.383 | 30.041 | 1.00 | 50.00 | N |
| ATOM | 3435 | CA | TYR | C | 44 | 6.617 | 42.519 | 29.258 | 1.00 | 50.00 | C |
| ATOM | 3436 | C | TYR | C | 44 | 8.053 | 43.004 | 29.426 | 1.00 | 50.00 | C |
| ATOM | 3437 | O | TYR | C | 44 | 8.321 | 44.176 | 29.674 | 1.00 | 50.00 | O |
| ATOM | 3438 | CB | TYR | C | 44 | 6.101 | 42.455 | 27.817 | 1.00 | 50.00 | C |
| ATOM | 3439 | CG | TYR | C | 44 | 6.978 | 41.591 | 26.938 | 1.00 | 50.00 | C |
| ATOM | 3440 | CD1 | TYR | C | 44 | 7.202 | 40.340 | 27.392 | 1.00 | 50.00 | C |
| ATOM | 3441 | CD2 | TYR | C | 44 | 7.941 | 42.230 | 26.242 | 1.00 | 50.00 | C |
| ATOM | 3442 | CE1 | TYR | C | 44 | 8.462 | 39.864 | 27.374 | 1.00 | 50.00 | C |
| ATOM | 3443 | CE2 | TYR | C | 44 | 9.116 | 41.616 | 26.002 | 1.00 | 50.00 | C |
| ATOM | 3444 | CZ | TYR | C | 44 | 9.395 | 40.436 | 26.587 | 1.00 | 50.00 | C |
| ATOM | 3445 | OH | TYR | C | 44 | 10.548 | 39.781 | 26.245 | 1.00 | 50.00 | O |
| ATOM | 3446 | N | LEU | C | 45 | 9.002 | 42.109 | 29.224 | 1.00 | 50.00 | N |
| ATOM | 3447 | CA | LEU | C | 45 | 10.444 | 42.315 | 29.087 | 1.00 | 50.00 | C |
| ATOM | 3448 | C | LEU | C | 45 | 10.987 | 43.037 | 27.855 | 1.00 | 50.00 | C |
| ATOM | 3449 | O | LEU | C | 45 | 10.248 | 43.907 | 27.398 | 1.00 | 50.00 | O |
| ATOM | 3450 | CB | LEU | C | 45 | 10.957 | 40.953 | 29.556 | 1.00 | 50.00 | C |
| ATOM | 3451 | CG | LEU | C | 45 | 10.820 | 40.534 | 31.019 | 1.00 | 50.00 | C |
| ATOM | 3452 | CD1 | LEU | C | 45 | 9.429 | 40.331 | 31.620 | 1.00 | 50.00 | C |
| ATOM | 3453 | CD2 | LEU | C | 45 | 11.737 | 39.449 | 31.572 | 1.00 | 50.00 | C |
| ATOM | 3454 | N | SER | C | 46 | 12.161 | 42.795 | 27.288 | 1.00 | 50.00 | N |
| ATOM | 3455 | CA | SER | C | 46 | 12.572 | 43.268 | 25.961 | 1.00 | 50.00 | C |
| ATOM | 3456 | C | SER | C | 46 | 13.950 | 42.770 | 25.545 | 1.00 | 50.00 | C |
| ATOM | 3457 | O | SER | C | 46 | 14.833 | 42.625 | 26.390 | 1.00 | 50.00 | O |
| ATOM | 3458 | CB | SER | C | 46 | 12.709 | 44.778 | 25.763 | 1.00 | 50.00 | C |
| ATOM | 3459 | OG | SER | C | 46 | 11.432 | 45.391 | 25.710 | 1.00 | 50.00 | O |
| ATOM | 3460 | N | ALA | C | 47 | 14.160 | 42.566 | 24.252 | 1.00 | 50.00 | N |
| ATOM | 3461 | CA | ALA | C | 47 | 15.389 | 41.959 | 23.739 | 1.00 | 50.00 | C |
| ATOM | 3462 | C | ALA | C | 47 | 15.538 | 41.815 | 22.228 | 1.00 | 50.00 | C |
| ATOM | 3463 | O | ALA | C | 47 | 14.573 | 41.884 | 21.468 | 1.00 | 50.00 | O |
| ATOM | 3464 | CB | ALA | C | 47 | 15.371 | 40.551 | 24.322 | 1.00 | 50.00 | C |
| ATOM | 3465 | N | LEU | C | 48 | 16.779 | 41.638 | 21.802 | 1.00 | 50.00 | N |
| ATOM | 3466 | CA | LEU | C | 48 | 17.245 | 41.868 | 20.434 | 1.00 | 50.00 | C |
| ATOM | 3467 | C | LEU | C | 48 | 17.711 | 40.564 | 19.799 | 1.00 | 50.00 | C |
| ATOM | 3468 | O | LEU | C | 48 | 18.328 | 39.733 | 20.464 | 1.00 | 50.00 | O |
| ATOM | 3469 | CB | LEU | C | 48 | 18.374 | 42.899 | 20.389 | 1.00 | 50.00 | C |
| ATOM | 3470 | CG | LEU | C | 48 | 17.973 | 44.370 | 20.502 | 1.00 | 50.00 | C |
| ATOM | 3471 | CD1 | LEU | C | 48 | 17.177 | 44.699 | 21.762 | 1.00 | 50.00 | C |
| ATOM | 3472 | CD2 | LEU | C | 48 | 19.191 | 45.291 | 20.527 | 1.00 | 50.00 | C |
| ATOM | 3473 | N | ARG | C | 49 | 17.418 | 40.378 | 18.522 | 1.00 | 50.00 | N |
| ATOM | 3474 | CA | ARG | C | 49 | 17.540 | 39.075 | 17.869 | 1.00 | 50.00 | C |
| ATOM | 3475 | C | ARG | C | 49 | 18.072 | 39.171 | 16.447 | 1.00 | 50.00 | C |
| ATOM | 3476 | O | ARG | C | 49 | 18.004 | 40.204 | 15.782 | 1.00 | 50.00 | O |
| ATOM | 3477 | CB | ARG | C | 49 | 16.227 | 38.295 | 17.802 | 1.00 | 50.00 | C |
| ATOM | 3478 | CG | ARG | C | 49 | 15.654 | 38.101 | 19.200 | 1.00 | 50.00 | C |
| ATOM | 3479 | CD | ARG | C | 49 | 14.621 | 36.984 | 19.265 | 1.00 | 50.00 | C |
| ATOM | 3480 | NE | ARG | C | 49 | 13.914 | 37.156 | 20.532 | 1.00 | 50.00 | N |
| ATOM | 3481 | CZ | ARG | C | 49 | 12.948 | 36.387 | 20.998 | 1.00 | 50.00 | C |
| ATOM | 3482 | NH1 | ARG | C | 49 | 12.599 | 35.323 | 20.312 | 1.00 | 50.00 | N |
| ATOM | 3483 | NH2 | ARG | C | 49 | 12.269 | 36.685 | 22.091 | 1.00 | 50.00 | N |
| ATOM | 3484 | N | THR | C | 50 | 18.552 | 38.022 | 16.003 | 1.00 | 50.00 | N |
| ATOM | 3485 | CA | THR | C | 50 | 19.053 | 37.841 | 14.643 | 1.00 | 50.00 | C |
| ATOM | 3486 | C | THR | C | 50 | 18.714 | 36.418 | 14.221 | 1.00 | 50.00 | C |
| ATOM | 3487 | O | THR | C | 50 | 19.210 | 35.488 | 14.856 | 1.00 | 50.00 | O |
| ATOM | 3488 | CB | THR | C | 50 | 20.570 | 38.036 | 14.640 | 1.00 | 50.00 | C |
| ATOM | 3489 | OG1 | THR | C | 50 | 20.967 | 39.184 | 15.375 | 1.00 | 50.00 | O |
| ATOM | 3490 | CG2 | THR | C | 50 | 21.078 | 38.054 | 13.200 | 1.00 | 50.00 | C |
| ATOM | 3491 | N | GLY | C | 51 | 18.056 | 36.247 | 13.086 | 1.00 | 50.00 | N |
| ATOM | 3492 | CA | GLY | C | 51 | 17.977 | 34.952 | 12.410 | 1.00 | 50.00 | C |
| ATOM | 3493 | C | GLY | C | 51 | 19.218 | 34.560 | 11.618 | 1.00 | 50.00 | C |
| ATOM | 3494 | O | GLY | C | 51 | 19.201 | 34.505 | 10.389 | 1.00 | 50.00 | O |
| ATOM | 3495 | N | TRP | C | 52 | 20.279 | 34.277 | 12.354 | 1.00 | 50.00 | N |
| ATOM | 3496 | CA | TRP | C | 52 | 21.620 | 34.004 | 11.839 | 1.00 | 50.00 | C |
| ATOM | 3497 | C | TRP | C | 52 | 22.010 | 32.537 | 11.990 | 1.00 | 50.00 | C |
| ATOM | 3498 | O | TRP | C | 52 | 22.569 | 32.007 | 11.031 | 1.00 | 50.00 | O |
| ATOM | 3499 | CB | TRP | C | 52 | 22.538 | 34.996 | 12.552 | 1.00 | 50.00 | C |
| ATOM | 3500 | CG | TRP | C | 52 | 24.021 | 34.779 | 12.374 | 1.00 | 50.00 | C |
| ATOM | 3501 | CD1 | TRP | C | 52 | 24.954 | 35.319 | 11.501 | 1.00 | 50.00 | C |
| ATOM | 3502 | CD2 | TRP | C | 52 | 24.736 | 33.806 | 13.045 | 1.00 | 50.00 | C |
| ATOM | 3503 | NE1 | TRP | C | 52 | 26.117 | 34.580 | 11.412 | 1.00 | 50.00 | N |
| ATOM | 3504 | CE2 | TRP | C | 52 | 25.900 | 33.566 | 12.322 | 1.00 | 50.00 | C |
| ATOM | 3505 | CE3 | TRP | C | 52 | 24.500 | 33.165 | 14.207 | 1.00 | 50.00 | C |
| ATOM | 3506 | CZ2 | TRP | C | 52 | 26.685 | 32.538 | 12.697 | 1.00 | 50.00 | C |
| ATOM | 3507 | CZ3 | TRP | C | 52 | 25.377 | 32.242 | 14.642 | 1.00 | 50.00 | C |
| ATOM | 3508 | CH2 | TRP | C | 52 | 26.465 | 31.965 | 13.899 | 1.00 | 50.00 | C |
| ATOM | 3509 | N | TYR | C | 53 | 21.813 | 31.891 | 13.131 | 1.00 | 50.00 | N |
| ATOM | 3510 | CA | TYR | C | 53 | 22.129 | 30.478 | 13.350 | 1.00 | 50.00 | C |
| ATOM | 3511 | C | TYR | C | 53 | 21.313 | 29.634 | 12.377 | 1.00 | 50.00 | C |
| ATOM | 3512 | O | TYR | C | 53 | 20.266 | 30.003 | 11.845 | 1.00 | 50.00 | O |
| ATOM | 3513 | CB | TYR | C | 53 | 22.014 | 29.993 | 14.798 | 1.00 | 50.00 | C |
| ATOM | 3514 | CG | TYR | C | 53 | 22.423 | 28.575 | 15.146 | 1.00 | 50.00 | C |
| ATOM | 3515 | CD1 | TYR | C | 53 | 23.695 | 28.293 | 15.495 | 1.00 | 50.00 | C |
| ATOM | 3516 | CD2 | TYR | C | 53 | 21.549 | 27.557 | 15.007 | 1.00 | 50.00 | C |
| ATOM | 3517 | CE1 | TYR | C | 53 | 24.096 | 27.017 | 15.666 | 1.00 | 50.00 | C |
| ATOM | 3518 | CE2 | TYR | C | 53 | 21.930 | 26.281 | 15.214 | 1.00 | 50.00 | C |
| ATOM | 3519 | CZ | TYR | C | 53 | 23.210 | 26.011 | 15.536 | 1.00 | 50.00 | C |
| ATOM | 3520 | OH | TYR | C | 53 | 23.604 | 24.707 | 15.686 | 1.00 | 50.00 | O |
| ATOM | 3521 | N | THR | C | 54 | 21.918 | 28.500 | 12.079 | 1.00 | 50.00 | N |
| ATOM | 3522 | CA | THR | C | 54 | 21.518 | 27.608 | 10.994 | 1.00 | 50.00 | C |
| ATOM | 3523 | C | THR | C | 54 | 21.958 | 26.207 | 11.402 | 1.00 | 50.00 | C |
| ATOM | 3524 | O | THR | C | 54 | 22.999 | 25.972 | 12.014 | 1.00 | 50.00 | O |
| ATOM | 3525 | CB | THR | C | 54 | 22.066 | 28.113 | 9.657 | 1.00 | 50.00 | C |
| ATOM | 3526 | OG1 | THR | C | 54 | 21.852 | 27.149 | 8.636 | 1.00 | 50.00 | O |
| ATOM | 3527 | CG2 | THR | C | 54 | 23.530 | 28.550 | 9.625 | 1.00 | 50.00 | C |
| ATOM | 3528 | N | SER | C | 55 | 21.115 | 25.264 | 11.018 | 1.00 | 50.00 | N |
| ATOM | 3529 | CA | SER | C | 55 | 21.359 | 23.831 | 11.178 | 1.00 | 50.00 | C |
| ATOM | 3530 | C | SER | C | 55 | 20.614 | 23.036 | 10.111 | 1.00 | 50.00 | C |
| ATOM | 3531 | O | SER | C | 55 | 19.823 | 23.559 | 9.326 | 1.00 | 50.00 | O |
| ATOM | 3532 | CB | SER | C | 55 | 21.028 | 23.403 | 12.607 | 1.00 | 50.00 | C |
| ATOM | 3533 | OG | SER | C | 55 | 22.122 | 23.657 | 13.476 | 1.00 | 50.00 | O |
| ATOM | 3534 | N | VAL | C | 56 | 20.900 | 21.745 | 10.069 | 1.00 | 50.00 | N |
| ATOM | 3535 | CA | VAL | C | 56 | 20.314 | 20.793 | 9.124 | 1.00 | 50.00 | C |
| ATOM | 3536 | C | VAL | C | 56 | 19.972 | 19.517 | 9.889 | 1.00 | 50.00 | C |
| ATOM | 3537 | O | VAL | C | 56 | 20.759 | 18.979 | 10.668 | 1.00 | 50.00 | O |
| ATOM | 3538 | CB | VAL | C | 56 | 21.219 | 20.606 | 7.902 | 1.00 | 50.00 | C |
| ATOM | 3539 | CG1 | VAL | C | 56 | 20.790 | 19.491 | 6.948 | 1.00 | 50.00 | C |
| ATOM | 3540 | CG2 | VAL | C | 56 | 21.325 | 21.872 | 7.052 | 1.00 | 50.00 | C |
| ATOM | 3541 | N | ILE | C | 57 | 18.787 | 19.004 | 9.598 | 1.00 | 50.00 | N |
| ATOM | 3542 | CA | ILE | C | 57 | 18.278 | 17.683 | 9.970 | 1.00 | 50.00 | C |
| ATOM | 3543 | C | ILE | C | 57 | 18.150 | 16.966 | 8.630 | 1.00 | 50.00 | C |
| ATOM | 3544 | O | ILE | C | 57 | 17.421 | 17.403 | 7.740 | 1.00 | 50.00 | O |
| ATOM | 3545 | CB | ILE | C | 57 | 16.959 | 17.720 | 10.751 | 1.00 | 50.00 | C |
| ATOM | 3546 | CG1 | ILE | C | 57 | 17.068 | 18.306 | 12.160 | 1.00 | 50.00 | C |
| ATOM | 3547 | CG2 | ILE | C | 57 | 16.175 | 16.408 | 10.784 | 1.00 | 50.00 | C |
| ATOM | 3548 | CD1 | ILE | C | 57 | 15.755 | 18.409 | 12.937 | 1.00 | 50.00 | C |
| ATOM | 3549 | N | THR | C | 58 | 18.784 | 15.810 | 8.530 | 1.00 | 50.00 | N |
| ATOM | 3550 | CA | THR | C | 58 | 18.663 | 14.902 | 7.390 | 1.00 | 50.00 | C |
| ATOM | 3551 | C | THR | C | 58 | 17.845 | 13.691 | 7.824 | 1.00 | 50.00 | C |
| ATOM | 3552 | O | THR | C | 58 | 18.344 | 12.837 | 8.555 | 1.00 | 50.00 | O |
| ATOM | 3553 | CB | THR | C | 58 | 20.059 | 14.540 | 6.880 | 1.00 | 50.00 | C |
| ATOM | 3554 | OG1 | THR | C | 58 | 20.665 | 15.690 | 6.307 | 1.00 | 50.00 | O |
| ATOM | 3555 | CG2 | THR | C | 58 | 20.098 | 13.433 | 5.827 | 1.00 | 50.00 | C |
| ATOM | 3556 | N | ILE | C | 59 | 16.638 | 13.548 | 7.304 | 1.00 | 50.00 | N |
| ATOM | 3557 | CA | ILE | C | 59 | 15.763 | 12.399 | 7.542 | 1.00 | 50.00 | C |
| ATOM | 3558 | C | ILE | C | 59 | 16.087 | 11.282 | 6.557 | 1.00 | 50.00 | C |
| ATOM | 3559 | O | ILE | C | 59 | 16.365 | 11.479 | 5.375 | 1.00 | 50.00 | O |
| ATOM | 3560 | CB | ILE | C | 59 | 14.290 | 12.809 | 7.583 | 1.00 | 50.00 | C |
| ATOM | 3561 | CG1 | ILE | C | 59 | 14.017 | 13.749 | 8.760 | 1.00 | 50.00 | C |
| ATOM | 3562 | CG2 | ILE | C | 59 | 13.260 | 11.683 | 7.667 | 1.00 | 50.00 | C |
| ATOM | 3563 | CD1 | ILE | C | 59 | 12.871 | 14.707 | 8.466 | 1.00 | 50.00 | C |
| ATOM | 3564 | N | GLU | C | 60 | 16.006 | 10.085 | 7.111 | 1.00 | 50.00 | N |
| ATOM | 3565 | CA | GLU | C | 60 | 16.134 | 8.823 | 6.388 | 1.00 | 50.00 | C |
| ATOM | 3566 | C | GLU | C | 60 | 14.781 | 8.141 | 6.545 | 1.00 | 50.00 | C |
| ATOM | 3567 | O | GLU | C | 60 | 14.348 | 7.726 | 7.620 | 1.00 | 50.00 | O |
| ATOM | 3568 | CB | GLU | C | 60 | 17.287 | 8.013 | 6.979 | 1.00 | 50.00 | C |
| ATOM | 3569 | CG | GLU | C | 60 | 18.707 | 8.559 | 6.850 | 1.00 | 50.00 | C |
| ATOM | 3570 | CD | GLU | C | 60 | 19.309 | 8.587 | 5.455 | 1.00 | 50.00 | C |
| ATOM | 3571 | OE1 | GLU | C | 60 | 19.430 | 9.695 | 4.888 | 1.00 | 50.00 | O |
| ATOM | 3572 | OE2 | GLU | C | 60 | 19.697 | 7.517 | 4.938 | 1.00 | 50.00 | O |
| ATOM | 3573 | N | LEU | C | 61 | 14.091 | 8.082 | 5.420 | 1.00 | 50.00 | N |
| ATOM | 3574 | CA | LEU | C | 61 | 12.723 | 7.577 | 5.303 | 1.00 | 50.00 | C |
| ATOM | 3575 | C | LEU | C | 61 | 12.517 | 6.069 | 5.318 | 1.00 | 50.00 | C |
| ATOM | 3576 | O | LEU | C | 61 | 11.523 | 5.606 | 5.878 | 1.00 | 50.00 | O |
| ATOM | 3577 | CB | LEU | C | 61 | 12.178 | 8.130 | 3.985 | 1.00 | 50.00 | C |
| ATOM | 3578 | CG | LEU | C | 61 | 12.154 | 9.645 | 3.790 | 1.00 | 50.00 | C |
| ATOM | 3579 | CD1 | LEU | C | 61 | 11.906 | 9.938 | 2.311 | 1.00 | 50.00 | C |
| ATOM | 3580 | CD2 | LEU | C | 61 | 11.188 | 10.161 | 4.854 | 1.00 | 50.00 | C |
| ATOM | 3581 | N | SER | C | 62 | 13.413 | 5.330 | 4.686 | 1.00 | 50.00 | N |
| ATOM | 3582 | CA | SER | C | 62 | 13.380 | 3.869 | 4.654 | 1.00 | 50.00 | C |
| ATOM | 3583 | C | SER | C | 62 | 14.164 | 3.379 | 5.866 | 1.00 | 50.00 | C |
| ATOM | 3584 | O | SER | C | 62 | 13.573 | 3.327 | 6.944 | 1.00 | 50.00 | O |
| ATOM | 3585 | CB | SER | C | 62 | 13.819 | 3.408 | 3.265 | 1.00 | 50.00 | C |
| ATOM | 3586 | OG | SER | C | 62 | 12.909 | 3.832 | 2.260 | 1.00 | 50.00 | O |
| ATOM | 3587 | N | ASN | C | 63 | 15.440 | 3.058 | 5.721 | 1.00 | 50.00 | N |
| ATOM | 3588 | CA | ASN | C | 63 | 16.298 | 2.488 | 6.763 | 1.00 | 50.00 | C |
| ATOM | 3589 | C | ASN | C | 63 | 16.165 | 0.995 | 7.032 | 1.00 | 50.00 | C |
| ATOM | 3590 | O | ASN | C | 63 | 17.161 | 0.335 | 7.328 | 1.00 | 50.00 | O |
| ATOM | 3591 | CB | ASN | C | 63 | 16.337 | 3.156 | 8.137 | 1.00 | 50.00 | C |
| ATOM | 3592 | CG | ASN | C | 63 | 16.906 | 4.565 | 8.134 | 1.00 | 50.00 | C |
| ATOM | 3593 | OD1 | ASN | C | 63 | 19.089 | 4.796 | 7.886 | 1.00 | 50.00 | O |
| ATOM | 3594 | ND2 | ASN | C | 63 | 16.044 | 5.520 | 8.422 | 1.00 | 50.00 | N |
| ATOM | 3595 | N | ILE | C | 64 | 14.941 | 0.496 | 7.043 | 1.00 | 50.00 | N |
| ATOM | 3596 | CA | ILE | C | 64 | 14.601 | −0.828 | 7.558 | 1.00 | 50.00 | C |
| ATOM | 3597 | C | ILE | C | 64 | 15.036 | −1.831 | 6.494 | 1.00 | 50.00 | C |
| ATOM | 3598 | O | ILE | C | 64 | 14.647 | −1.764 | 5.328 | 1.00 | 50.00 | O |
| ATOM | 3599 | CB | ILE | C | 64 | 13.132 | −0.805 | 7.992 | 1.00 | 50.00 | C |
| ATOM | 3600 | CG1 | ILE | C | 64 | 12.764 | 0.131 | 9.147 | 1.00 | 50.00 | C |
| ATOM | 3601 | CG2 | ILE | C | 64 | 12.659 | −2.245 | 8.191 | 1.00 | 50.00 | C |
| ATOM | 3602 | CD1 | ILE | C | 64 | 11.282 | 0.317 | 9.470 | 1.00 | 50.00 | C |
| ATOM | 3603 | N | LYS | C | 65 | 15.861 | −2.764 | 6.939 | 1.00 | 50.00 | N |
| ATOM | 3604 | CA | LYS | C | 65 | 16.609 | −3.722 | 6.123 | 1.00 | 50.00 | C |
| ATOM | 3605 | C | LYS | C | 65 | 15.870 | −4.663 | 5.180 | 1.00 | 50.00 | C |
| ATOM | 3606 | O | LYS | C | 65 | 16.466 | −5.088 | 4.191 | 1.00 | 50.00 | O |
| ATOM | 3607 | CB | LYS | C | 65 | 17.472 | −4.595 | 7.036 | 1.00 | 50.00 | C |
| ATOM | 3608 | CG | LYS | C | 65 | 18.470 | −3.833 | 7.906 | 1.00 | 50.00 | C |
| ATOM | 3609 | CD | LYS | C | 65 | 19.322 | −4.731 | 8.800 | 1.00 | 50.00 | C |
| ATOM | 3610 | CE | LYS | C | 65 | 20.345 | −3.883 | 9.552 | 1.00 | 50.00 | C |
| ATOM | 3611 | NZ | LYS | C | 65 | 21.263 | −4.720 | 10.338 | 1.00 | 50.00 | N |
| ATOM | 3612 | N | LYS | C | 66 | 14.639 | −5.040 | 5.482 | 1.00 | 50.00 | N |
| ATOM | 3613 | CA | LYS | C | 66 | 13.854 | −5.986 | 4.688 | 1.00 | 50.00 | C |
| ATOM | 3614 | C | LYS | C | 66 | 14.289 | −7.447 | 4.642 | 1.00 | 50.00 | C |
| ATOM | 3615 | O | LYS | C | 66 | 14.009 | −8.167 | 3.683 | 1.00 | 50.00 | O |
| ATOM | 3616 | CB | LYS | C | 66 | 13.540 | −5.524 | 3.266 | 1.00 | 50.00 | C |
| ATOM | 3617 | CG | LYS | C | 66 | 12.863 | −4.171 | 3.064 | 1.00 | 50.00 | C |
| ATOM | 3618 | CD | LYS | C | 66 | 12.910 | −3.874 | 1.566 | 1.00 | 50.00 | C |
| ATOM | 3619 | CE | LYS | C | 66 | 12.334 | −2.504 | 1.221 | 1.00 | 50.00 | C |
| ATOM | 3620 | NZ | LYS | C | 66 | 12.060 | −2.329 | −0.211 | 1.00 | 50.00 | N |
| ATOM | 3621 | N | ASN | C | 67 | 14.935 | −7.955 | 5.677 | 1.00 | 50.00 | N |
| ATOM | 3622 | CA | ASN | C | 67 | 15.590 | −9.262 | 5.639 | 1.00 | 50.00 | C |
| ATOM | 3623 | C | ASN | C | 67 | 14.572 | −10.372 | 5.881 | 1.00 | 50.00 | C |
| ATOM | 3624 | O | ASN | C | 67 | 13.926 | −10.379 | 6.928 | 1.00 | 50.00 | O |
| ATOM | 3625 | CB | ASN | C | 67 | 16.820 | −9.178 | 6.544 | 1.00 | 50.00 | C |
| ATOM | 3626 | CG | ASN | C | 67 | 16.639 | −9.118 | 8.054 | 1.00 | 50.00 | C |
| ATOM | 3627 | OD1 | ASN | C | 67 | 16.943 | −10.092 | 8.741 | 1.00 | 50.00 | O |
| ATOM | 3628 | ND2 | ASN | C | 67 | 16.156 | −8.020 | 8.607 | 1.00 | 50.00 | N |
| ATOM | 3629 | N | LYS | C | 68 | 14.397 | −11.242 | 4.898 | 1.00 | 50.00 | N |
| ATOM | 3630 | CA | LYS | C | 68 | 13.292 | −12.195 | 4.764 | 1.00 | 50.00 | C |
| ATOM | 3631 | C | LYS | C | 68 | 12.176 | −11.620 | 3.895 | 1.00 | 50.00 | C |
| ATOM | 3632 | O | LYS | C | 68 | 12.270 | −10.508 | 3.377 | 1.00 | 50.00 | O |
| ATOM | 3633 | CB | LYS | C | 68 | 12.873 | −12.931 | 6.040 | 1.00 | 50.00 | C |
| ATOM | 3634 | CG | LYS | C | 68 | 14.016 | −13.663 | 6.741 | 1.00 | 50.00 | C |
| ATOM | 3635 | CD | LYS | C | 68 | 13.707 | −14.232 | 8.125 | 1.00 | 50.00 | C |
| ATOM | 3636 | CE | LYS | C | 68 | 14.967 | −14.869 | 8.710 | 1.00 | 50.00 | C |
| ATOM | 3637 | NZ | LYS | C | 68 | 14.712 | −15.551 | 9.987 | 1.00 | 50.00 | N |
| ATOM | 3638 | N | CYS | C | 69 | 11.092 | −12.330 | 3.626 | 1.00 | 50.00 | N |
| ATOM | 3639 | CA | CYS | C | 69 | 9.745 | −11.950 | 4.055 | 1.00 | 50.00 | C |
| ATOM | 3640 | C | CYS | C | 69 | 9.326 | −10.497 | 3.863 | 1.00 | 50.00 | C |
| ATOM | 3641 | O | CYS | C | 69 | 8.485 | −10.215 | 3.009 | 1.00 | 50.00 | O |
| ATOM | 3642 | CB | CYS | C | 69 | 9.454 | −12.546 | 5.433 | 1.00 | 50.00 | C |
| ATOM | 3643 | SG | CYS | C | 69 | 9.632 | −14.330 | 5.277 | 1.00 | 50.00 | S |
| ATOM | 3644 | N | ASN | C | 70 | 9.867 | −9.620 | 4.693 | 1.00 | 50.00 | N |
| ATOM | 3645 | CA | ASN | C | 70 | 9.657 | −8.171 | 4.716 | 1.00 | 50.00 | C |
| ATOM | 3646 | C | ASN | C | 70 | 9.348 | −7.399 | 3.439 | 1.00 | 50.00 | C |
| ATOM | 3647 | O | ASN | C | 70 | 8.567 | −6.451 | 3.510 | 1.00 | 50.00 | O |
| ATOM | 3648 | CB | ASN | C | 70 | 10.960 | −7.564 | 5.233 | 1.00 | 50.00 | C |
| ATOM | 3649 | CG | ASN | C | 70 | 11.309 | −7.522 | 6.712 | 1.00 | 50.00 | C |
| ATOM | 3650 | OD1 | ASN | C | 70 | 11.752 | −8.492 | 7.324 | 1.00 | 50.00 | O |
| ATOM | 3651 | ND2 | ASN | C | 70 | 11.216 | −6.326 | 7.264 | 1.00 | 50.00 | N |
| ATOM | 3652 | N | GLY | C | 71 | 10.032 | −7.726 | 2.354 | 1.00 | 50.00 | N |
| ATOM | 3653 | CA | GLY | C | 71 | 9.890 | −7.093 | 1.043 | 1.00 | 50.00 | C |
| ATOM | 3654 | C | GLY | C | 71 | 8.495 | −6.663 | 0.613 | 1.00 | 50.00 | C |
| ATOM | 3655 | O | GLY | C | 71 | 8.294 | −5.482 | 0.335 | 1.00 | 50.00 | O |
| ATOM | 3656 | N | THR | C | 72 | 7.555 | −7.588 | 0.712 | 1.00 | 50.00 | N |
| ATOM | 3657 | CA | THR | C | 72 | 6.107 | −7.373 | 0.726 | 1.00 | 50.00 | C |
| ATOM | 3658 | C | THR | C | 72 | 5.602 | −5.990 | 1.122 | 1.00 | 50.00 | C |
| ATOM | 3659 | O | THR | C | 72 | 5.068 | −5.250 | 0.297 | 1.00 | 50.00 | O |
| ATOM | 3660 | CB | THR | C | 72 | 5.582 | −8.561 | 1.538 | 1.00 | 50.00 | C |
| ATOM | 3661 | OG1 | THR | C | 72 | 5.808 | −9.777 | 0.840 | 1.00 | 50.00 | O |
| ATOM | 3662 | CG2 | THR | C | 72 | 4.077 | −8.600 | 1.779 | 1.00 | 50.00 | C |
| ATOM | 3663 | N | ASP | C | 73 | 5.801 | −5.622 | 2.375 | 1.00 | 50.00 | N |
| ATOM | 3664 | CA | ASP | C | 73 | 5.123 | −4.483 | 2.992 | 1.00 | 50.00 | C |
| ATOM | 3665 | C | ASP | C | 73 | 5.882 | −3.175 | 2.863 | 1.00 | 50.00 | C |
| ATOM | 3666 | O | ASP | C | 73 | 5.356 | −2.248 | 2.253 | 1.00 | 50.00 | O |
| ATOM | 3667 | CB | ASP | C | 73 | 4.879 | −4.838 | 4.456 | 1.00 | 50.00 | C |
| ATOM | 3668 | CG | ASP | C | 73 | 3.747 | −5.849 | 4.398 | 1.00 | 50.00 | C |
| ATOM | 3669 | OD1 | ASP | C | 73 | 2.603 | −5.433 | 4.118 | 1.00 | 50.00 | O |
| ATOM | 3670 | OD2 | ASP | C | 73 | 4.017 | −7.053 | 4.597 | 1.00 | 50.00 | O |
| ATOM | 3671 | N | ALA | C | 74 | 7.134 | −3.152 | 3.282 | 1.00 | 50.00 | N |
| ATOM | 3672 | CA | ALA | C | 74 | 8.136 | −2.247 | 2.725 | 1.00 | 50.00 | C |
| ATOM | 3673 | C | ALA | C | 74 | 7.997 | −1.779 | 1.280 | 1.00 | 50.00 | C |
| ATOM | 3674 | O | ALA | C | 74 | 7.996 | −0.566 | 1.078 | 1.00 | 50.00 | O |
| ATOM | 3675 | CB | ALA | C | 74 | 9.419 | −3.023 | 3.004 | 1.00 | 50.00 | C |
| ATOM | 3676 | N | LYS | C | 75 | 7.700 | −2.621 | 0.302 | 1.00 | 50.00 | N |
| ATOM | 3677 | CA | LYS | C | 75 | 7.234 | −2.201 | −1.021 | 1.00 | 50.00 | C |
| ATOM | 3678 | C | LYS | C | 75 | 6.079 | −1.210 | −0.996 | 1.00 | 50.00 | C |
| ATOM | 3679 | O | LYS | C | 75 | 6.359 | −0.048 | −1.284 | 1.00 | 50.00 | O |
| ATOM | 3680 | CB | LYS | C | 75 | 6.860 | −3.336 | −1.973 | 1.00 | 50.00 | C |
| ATOM | 3681 | CG | LYS | C | 75 | 8.068 | −4.114 | −2.486 | 1.00 | 50.00 | C |
| ATOM | 3682 | CD | LYS | C | 75 | 7.623 | −5.328 | −3.297 | 1.00 | 50.00 | C |
| ATOM | 3683 | CE | LYS | C | 75 | 8.812 | −6.255 | −3.536 | 1.00 | 50.00 | C |
| ATOM | 3684 | NZ | LYS | C | 75 | 8.429 | −7.320 | −4.474 | 1.00 | 50.00 | N |
| ATOM | 3685 | N | VAL | C | 76 | 4.888 | −1.583 | −0.558 | 1.00 | 50.00 | N |
| ATOM | 3686 | CA | VAL | C | 76 | 3.817 | −0.661 | −0.176 | 1.00 | 50.00 | C |
| ATOM | 3687 | C | VAL | C | 76 | 4.238 | −0.668 | 0.447 | 1.00 | 50.00 | C |
| ATOM | 3688 | O | VAL | C | 76 | 3.776 | −1.729 | 0.035 | 1.00 | 50.00 | O |
| ATOM | 3689 | CB | VAL | C | 76 | 2.804 | −1.474 | 0.630 | 1.00 | 50.00 | C |
| ATOM | 3690 | CG1 | VAL | C | 76 | 1.709 | −0.528 | 1.110 | 1.00 | 50.00 | C |
| ATOM | 3691 | CG2 | VAL | C | 76 | 2.133 | −2.689 | −0.010 | 1.00 | 50.00 | C |
| ATOM | 3692 | N | LYS | C | 77 | 5.107 | 0.634 | 1.441 | 1.00 | 50.00 | N |
| ATOM | 3693 | CA | LYS | C | 77 | 5.592 | 1.794 | 2.189 | 1.00 | 50.00 | C |
| ATOM | 3694 | C | LYS | C | 77 | 6.397 | 2.775 | 1.355 | 1.00 | 50.00 | C |
| ATOM | 3695 | O | LYS | C | 77 | 5.924 | 3.875 | 1.077 | 1.00 | 50.00 | O |
| ATOM | 3696 | CB | LYS | C | 77 | 6.358 | 1.302 | 3.416 | 1.00 | 50.00 | C |
| ATOM | 3697 | CG | LYS | C | 77 | 5.446 | 0.368 | 4.204 | 1.00 | 50.00 | C |
| ATOM | 3698 | CD | LYS | C | 77 | 5.979 | −0.011 | 5.575 | 1.00 | 50.00 | C |
| ATOM | 3699 | CE | LYS | C | 77 | 4.810 | −0.755 | 6.211 | 1.00 | 50.00 | C |
| ATOM | 3700 | NZ | LYS | C | 77 | 4.922 | −1.074 | 7.634 | 1.00 | 50.00 | N |
| ATOM | 3701 | N | LEU | C | 78 | 7.534 | 2.300 | 0.881 | 1.00 | 50.00 | N |
| ATOM | 3702 | CA | LEU | C | 78 | 8.360 | 2.933 | −0.145 | 1.00 | 50.00 | C |
| ATOM | 3703 | C | LEU | C | 78 | 7.694 | 3.276 | −1.474 | 1.00 | 50.00 | C |
| ATOM | 3704 | O | LEU | C | 78 | 8.198 | 4.159 | −2.167 | 1.00 | 50.00 | O |
| ATOM | 3705 | CB | LEU | C | 78 | 9.531 | 1.967 | −0.325 | 1.00 | 50.00 | C |
| ATOM | 3706 | CG | LEU | C | 78 | 10.561 | 1.803 | 0.796 | 1.00 | 50.00 | C |
| ATOM | 3707 | CD1 | LEU | C | 78 | 10.166 | 1.170 | 2.130 | 1.00 | 50.00 | C |
| ATOM | 3708 | CD2 | LEU | C | 78 | 11.693 | 0.956 | 0.219 | 1.00 | 50.00 | C |
| ATOM | 3709 | N | ILE | C | 79 | 6.550 | 2.693 | −1.803 | 1.00 | 50.00 | N |
| ATOM | 3710 | CA | ILE | C | 79 | 5.565 | 3.219 | −2.750 | 1.00 | 50.00 | C |
| ATOM | 3711 | C | ILE | C | 79 | 5.078 | 4.525 | −2.131 | 1.00 | 50.00 | C |
| ATOM | 3712 | O | ILE | C | 79 | 5.590 | 5.571 | −2.524 | 1.00 | 50.00 | O |
| ATOM | 3713 | CB | ILE | C | 79 | 4.503 | 2.192 | −3.162 | 1.00 | 50.00 | C |
| ATOM | 3714 | CG1 | ILE | C | 79 | 5.049 | 0.961 | −3.889 | 1.00 | 50.00 | C |
| ATOM | 3715 | CG2 | ILE | C | 79 | 3.358 | 2.757 | −4.003 | 1.00 | 50.00 | C |
| ATOM | 3716 | CD1 | ILE | C | 79 | 4.141 | −0.266 | −3.949 | 1.00 | 50.00 | C |
| ATOM | 3717 | N | LYS | C | 80 | 4.186 | 4.516 | −1.156 | 1.00 | 50.00 | N |
| ATOM | 3718 | CA | LYS | C | 80 | 3.538 | 5.691 | −0.570 | 1.00 | 50.00 | C |
| ATOM | 3719 | C | LYS | C | 80 | 4.325 | 6.884 | −0.046 | 1.00 | 50.00 | C |
| ATOM | 3720 | O | LYS | C | 80 | 3.876 | 8.022 | −0.164 | 1.00 | 50.00 | O |
| ATOM | 3721 | CB | LYS | C | 80 | 2.702 | 5.193 | 0.606 | 1.00 | 50.00 | C |
| ATOM | 3722 | CG | LYS | C | 80 | 1.662 | 4.181 | 0.131 | 1.00 | 50.00 | C |
| ATOM | 3723 | CD | LYS | C | 80 | 0.400 | 4.145 | 0.980 | 1.00 | 50.00 | C |
| ATOM | 3724 | CE | LYS | C | 80 | −0.513 | 2.924 | 0.989 | 1.00 | 50.00 | C |
| ATOM | 3725 | NZ | LYS | C | 80 | −1.211 | 2.522 | 2.217 | 1.00 | 50.00 | N |
| ATOM | 3726 | N | GLN | C | 81 | 5.478 | 6.614 | 0.541 | 1.00 | 50.00 | N |
| ATOM | 3727 | CA | GLN | C | 81 | 6.592 | 7.547 | 0.706 | 1.00 | 50.00 | C |
| ATOM | 3728 | C | GLN | C | 81 | 6.924 | 8.412 | −0.505 | 1.00 | 50.00 | C |
| ATOM | 3729 | O | GLN | C | 81 | 7.035 | 9.630 | −0.374 | 1.00 | 50.00 | O |
| ATOM | 3730 | CB | GLN | C | 81 | 7.795 | 6.692 | 1.110 | 1.00 | 50.00 | C |
| ATOM | 3731 | CG | GLN | C | 81 | 7.737 | 5.997 | 2.469 | 1.00 | 50.00 | C |
| ATOM | 3732 | CD | GLN | C | 81 | 7.847 | 6.891 | 3.694 | 1.00 | 50.00 | C |
| ATOM | 3733 | OE1 | GLN | C | 81 | 7.915 | 8.118 | 3.638 | 1.00 | 50.00 | O |
| ATOM | 3734 | NE2 | GLN | C | 81 | 7.793 | 6.263 | 4.855 | 1.00 | 50.00 | N |
| ATOM | 3735 | N | GLU | C | 82 | 7.031 | 7.798 | −1.671 | 1.00 | 50.00 | N |
| ATOM | 3736 | CA | GLU | C | 82 | 7.067 | 8.466 | −2.971 | 1.00 | 50.00 | C |
| ATOM | 3737 | C | GLU | C | 82 | 5.798 | 9.115 | −3.502 | 1.00 | 50.00 | C |
| ATOM | 3738 | O | GLU | C | 82 | 5.888 | 10.180 | −4.111 | 1.00 | 50.00 | O |
| ATOM | 3739 | CB | GLU | C | 82 | 7.672 | 7.507 | −3.999 | 1.00 | 50.00 | C |
| ATOM | 3740 | CG | GLU | C | 82 | 9.176 | 7.309 | −3.818 | 1.00 | 50.00 | C |
| ATOM | 3741 | CD | GLU | C | 82 | 9.840 | 8.669 | −3.981 | 1.00 | 50.00 | C |
| ATOM | 3742 | OE1 | GLU | C | 82 | 10.350 | 9.228 | −2.986 | 1.00 | 50.00 | O |
| ATOM | 3743 | OE2 | GLU | C | 82 | 9.671 | 9.287 | −5.054 | 1.00 | 50.00 | O |
| ATOM | 3744 | N | LEU | C | 83 | 4.625 | 8.552 | −3.273 | 1.00 | 50.00 | N |
| ATOM | 3745 | CA | LEU | C | 83 | 3.353 | 9.187 | −3.617 | 1.00 | 50.00 | C |
| ATOM | 3746 | C | LEU | C | 83 | 3.049 | 10.413 | −2.759 | 1.00 | 50.00 | C |
| ATOM | 3747 | O | LEU | C | 83 | 2.504 | 11.375 | −3.300 | 1.00 | 50.00 | O |
| ATOM | 3748 | CB | LEU | C | 83 | 2.279 | 8.098 | −3.646 | 1.00 | 50.00 | C |
| ATOM | 3749 | CG | LEU | C | 83 | 2.316 | 7.000 | −4.713 | 1.00 | 50.00 | C |
| ATOM | 3750 | CD1 | LEU | C | 83 | 3.579 | 6.143 | −4.749 | 1.00 | 50.00 | C |
| ATOM | 3751 | CD2 | LEU | C | 83 | 1.216 | 5.969 | −4.470 | 1.00 | 50.00 | C |
| ATOM | 3752 | N | ASP | C | 84 | 3.453 | 10.453 | −1.497 | 1.00 | 50.00 | N |
| ATOM | 3753 | CA | ASP | C | 84 | 3.695 | 11.654 | −0.695 | 1.00 | 50.00 | C |
| ATOM | 3754 | C | ASP | C | 84 | 4.719 | 12.545 | −1.391 | 1.00 | 50.00 | C |
| ATOM | 3755 | O | ASP | C | 84 | 4.367 | 13.435 | −2.163 | 1.00 | 50.00 | O |
| ATOM | 3756 | CB | ASP | C | 84 | 4.160 | 11.314 | 0.724 | 1.00 | 50.00 | C |
| ATOM | 3757 | CG | ASP | C | 84 | 3.200 | 10.969 | 1.849 | 1.00 | 50.00 | C |
| ATOM | 3758 | OD1 | ASP | C | 84 | 2.334 | 11.819 | 2.147 | 1.00 | 50.00 | O |
| ATOM | 3759 | OD2 | ASP | C | 84 | 3.311 | 9.875 | 2.445 | 1.00 | 50.00 | O |
| ATOM | 3760 | N | LYS | C | 85 | 5.999 | 12.287 | −1.183 | 1.00 | 50.00 | N |
| ATOM | 3761 | CA | LYS | C | 85 | 7.060 | 13.281 | −1.329 | 1.00 | 50.00 | C |
| ATOM | 3762 | C | LYS | C | 85 | 7.471 | 13.717 | −2.726 | 1.00 | 50.00 | C |
| ATOM | 3763 | O | LYS | C | 85 | 8.139 | 14.746 | −2.816 | 1.00 | 50.00 | O |
| ATOM | 3764 | CB | LYS | C | 85 | 8.333 | 12.694 | −0.724 | 1.00 | 50.00 | C |
| ATOM | 3765 | CG | LYS | C | 85 | 8.204 | 12.668 | 0.796 | 1.00 | 50.00 | C |
| ATOM | 3766 | CD | LYS | C | 85 | 9.563 | 12.283 | 1.367 | 1.00 | 50.00 | C |
| ATOM | 3767 | CE | LYS | C | 85 | 9.442 | 12.433 | 2.880 | 1.00 | 50.00 | C |
| ATOM | 3768 | NZ | LYS | C | 85 | 10.818 | 12.697 | 3.314 | 1.00 | 50.00 | N |
| ATOM | 3769 | N | TYR | C | 86 | 7.125 | 12.969 | −3.759 | 1.00 | 50.00 | N |
| ATOM | 3770 | CA | TYR | C | 86 | 7.395 | 13.334 | −5.148 | 1.00 | 50.00 | C |
| ATOM | 3771 | C | TYR | C | 86 | 7.211 | 14.803 | −5.508 | 1.00 | 50.00 | C |
| ATOM | 3772 | O | TYR | C | 86 | 8.078 | 15.381 | −6.160 | 1.00 | 50.00 | O |
| ATOM | 3773 | CB | TYR | C | 86 | 6.584 | 12.379 | −6.025 | 1.00 | 50.00 | C |
| ATOM | 3774 | CG | TYR | C | 86 | 6.831 | 12.572 | −7.503 | 1.00 | 50.00 | C |
| ATOM | 3775 | CD1 | TYR | C | 86 | 8.011 | 12.223 | −8.056 | 1.00 | 50.00 | C |
| ATOM | 3776 | CD2 | TYR | C | 86 | 5.989 | 13.396 | −8.158 | 1.00 | 50.00 | C |
| ATOM | 3777 | CE1 | TYR | C | 86 | 8.362 | 12.712 | −9.263 | 1.00 | 50.00 | C |
| ATOM | 3778 | CE2 | TYR | C | 86 | 6.371 | 13.902 | −9.346 | 1.00 | 50.00 | C |
| ATOM | 3779 | CZ | TYR | C | 86 | 7.543 | 13.560 | −9.915 | 1.00 | 50.00 | C |
| ATOM | 3780 | OH | TYR | C | 86 | 7.945 | 14.181 | −11.069 | 1.00 | 50.00 | O |
| ATOM | 3781 | N | LYS | C | 87 | 6.116 | 15.411 | −5.091 | 1.00 | 50.00 | N |
| ATOM | 3782 | CA | LYS | C | 87 | 5.826 | 16.815 | −5.373 | 1.00 | 50.00 | C |
| ATOM | 3783 | C | LYS | C | 87 | 6.386 | 17.868 | −4.428 | 1.00 | 50.00 | C |
| ATOM | 3784 | O | LYS | C | 87 | 6.691 | 18.957 | −4.912 | 1.00 | 50.00 | O |
| ATOM | 3785 | CB | LYS | C | 87 | 4.317 | 16.937 | −5.218 | 1.00 | 50.00 | C |
| ATOM | 3786 | CG | LYS | C | 87 | 3.527 | 16.093 | −6.216 | 1.00 | 50.00 | C |
| ATOM | 3787 | CD | LYS | C | 87 | 2.109 | 16.554 | −5.910 | 1.00 | 50.00 | C |
| ATOM | 3788 | CE | LYS | C | 87 | 0.817 | 15.910 | −6.391 | 1.00 | 50.00 | C |
| ATOM | 3789 | NZ | LYS | C | 87 | −0.127 | 16.817 | −5.725 | 1.00 | 50.00 | N |
| ATOM | 3790 | N | ASN | C | 88 | 6.540 | 17.596 | −3.142 | 1.00 | 50.00 | N |
| ATOM | 3791 | CA | ASN | C | 88 | 7.567 | 18.256 | −2.334 | 1.00 | 50.00 | C |
| ATOM | 3792 | C | ASN | C | 88 | 8.933 | 18.320 | −2.998 | 1.00 | 50.00 | C |
| ATOM | 3793 | O | ASN | C | 88 | 9.402 | 19.437 | −3.205 | 1.00 | 50.00 | O |
| ATOM | 3794 | CB | ASN | C | 88 | 7.831 | 17.614 | −0.973 | 1.00 | 50.00 | C |
| ATOM | 3795 | CG | ASN | C | 88 | 6.702 | 17.910 | −0.006 | 1.00 | 50.00 | C |
| ATOM | 3796 | OD1 | ASN | C | 88 | 5.882 | 17.019 | 0.208 | 1.00 | 50.00 | O |
| ATOM | 3797 | ND2 | ASN | C | 88 | 6.640 | 19.156 | 0.429 | 1.00 | 50.00 | N |
| ATOM | 3798 | N | ALA | C | 89 | 9.459 | 17.187 | −3.435 | 1.00 | 50.00 | N |
| ATOM | 3799 | CA | ALA | C | 89 | 10.609 | 17.112 | −4.332 | 1.00 | 50.00 | C |
| ATOM | 3800 | C | ALA | C | 89 | 10.428 | 18.050 | −5.519 | 1.00 | 50.00 | C |
| ATOM | 3801 | O | ALA | C | 89 | 11.007 | 19.130 | −5.426 | 1.00 | 50.00 | O |
| ATOM | 3802 | CB | ALA | C | 89 | 10.931 | 15.653 | −4.654 | 1.00 | 50.00 | C |
| ATOM | 3803 | N | VAL | C | 90 | 9.544 | 17.833 | −6.479 | 1.00 | 50.00 | N |
| ATOM | 3804 | CA | VAL | C | 90 | 9.284 | 18.748 | −7.593 | 1.00 | 50.00 | C |
| ATOM | 3805 | C | VAL | C | 90 | 9.209 | 20.236 | −7.263 | 1.00 | 50.00 | C |
| ATOM | 3806 | O | VAL | C | 90 | 9.932 | 20.988 | −7.915 | 1.00 | 50.00 | O |
| ATOM | 3807 | CB | VAL | C | 90 | 8.090 | 18.322 | −8.450 | 1.00 | 50.00 | C |
| ATOM | 3808 | CG1 | VAL | C | 90 | 7.812 | 19.305 | −9.587 | 1.00 | 50.00 | C |
| ATOM | 3809 | CG2 | VAL | C | 90 | 8.243 | 16.930 | −9.062 | 1.00 | 50.00 | C |
| ATOM | 3810 | N | THR | C | 91 | 8.351 | 20.675 | −6.354 | 1.00 | 50.00 | N |
| ATOM | 3811 | CA | THR | C | 91 | 8.291 | 22.048 | −5.850 | 1.00 | 50.00 | C |
| ATOM | 3812 | C | THR | C | 91 | 9.697 | 22.550 | −5.546 | 1.00 | 50.00 | C |
| ATOM | 3813 | O | THR | C | 91 | 10.179 | 23.345 | −6.351 | 1.00 | 50.00 | O |
| ATOM | 3814 | CB | THR | C | 91 | 7.282 | 22.209 | −4.710 | 1.00 | 50.00 | C |
| ATOM | 3815 | OG1 | THR | C | 91 | 5.965 | 21.995 | −5.196 | 1.00 | 50.00 | O |
| ATOM | 3816 | CG2 | THR | C | 91 | 7.247 | 23.571 | −4.020 | 1.00 | 50.00 | C |
| ATOM | 3817 | N | GLU | C | 92 | 10.385 | 22.008 | −4.554 | 1.00 | 50.00 | N |
| ATOM | 3818 | CA | GLU | C | 92 | 11.815 | 22.207 | −4.311 | 1.00 | 50.00 | C |
| ATOM | 3819 | C | GLU | C | 92 | 12.689 | 22.518 | −5.520 | 1.00 | 50.00 | C |
| ATOM | 3820 | O | GLU | C | 92 | 13.166 | 23.645 | −5.636 | 1.00 | 50.00 | O |
| ATOM | 3821 | CB | GLU | C | 92 | 12.371 | 21.076 | −3.443 | 1.00 | 50.00 | C |
| ATOM | 3822 | CG | GLU | C | 92 | 11.732 | 20.925 | −2.063 | 1.00 | 50.00 | C |
| ATOM | 3823 | CD | GLU | C | 92 | 12.125 | 22.108 | −1.196 | 1.00 | 50.00 | C |
| ATOM | 3824 | OE1 | GLU | C | 92 | 11.247 | 22.818 | −0.659 | 1.00 | 50.00 | O |
| ATOM | 3825 | OE2 | GLU | C | 92 | 13.342 | 22.381 | −1.136 | 1.00 | 50.00 | O |
| ATOM | 3826 | N | LEU | C | 93 | 12.722 | 21.648 | −6.516 | 1.00 | 50.00 | N |
| ATOM | 3827 | CA | LEU | C | 93 | 13.395 | 21.863 | −7.797 | 1.00 | 50.00 | C |
| ATOM | 3828 | C | LEU | C | 93 | 13.022 | 23.181 | −8.469 | 1.00 | 50.00 | C |
| ATOM | 3829 | O | LEU | C | 93 | 13.883 | 24.059 | −8.483 | 1.00 | 50.00 | O |
| ATOM | 3830 | CB | LEU | C | 93 | 13.230 | 20.632 | −8.690 | 1.00 | 50.00 | C |
| ATOM | 3831 | CG | LEU | C | 93 | 13.896 | 19.307 | −8.310 | 1.00 | 50.00 | C |
| ATOM | 3832 | CD1 | LEU | C | 93 | 13.492 | 18.712 | −6.963 | 1.00 | 50.00 | C |
| ATOM | 3833 | CD2 | LEU | C | 93 | 13.532 | 18.238 | −9.339 | 1.00 | 50.00 | C |
| ATOM | 3834 | N | GLN | C | 94 | 11.780 | 23.412 | −8.865 | 1.00 | 50.00 | N |
| ATOM | 3835 | CA | GLN | C | 94 | 11.252 | 24.697 | −9.332 | 1.00 | 50.00 | C |
| ATOM | 3836 | C | GLN | C | 94 | 11.645 | 25.993 | −8.629 | 1.00 | 50.00 | C |
| ATOM | 3837 | O | GLN | C | 94 | 11.908 | 26.999 | −9.287 | 1.00 | 50.00 | O |
| ATOM | 3838 | CB | GLN | C | 94 | 9.726 | 24.607 | −9.405 | 1.00 | 50.00 | C |
| ATOM | 3839 | CG | GLN | C | 94 | 9.134 | 23.614 | −10.404 | 1.00 | 50.00 | C |
| ATOM | 3840 | CD | GLN | C | 94 | 9.367 | 23.946 | −11.871 | 1.00 | 50.00 | C |
| ATOM | 3841 | OE1 | GLN | C | 94 | 9.789 | 25.034 | −12.260 | 1.00 | 50.00 | O |
| ATOM | 3842 | NE2 | GLN | C | 94 | 9.086 | 22.994 | −12.741 | 1.00 | 50.00 | N |
| ATOM | 3843 | N | LEU | C | 95 | 11.695 | 25.972 | −7.307 | 1.00 | 50.00 | N |
| ATOM | 3844 | CA | LEU | C | 95 | 12.127 | 27.068 | −6.439 | 1.00 | 50.00 | C |
| ATOM | 3845 | C | LEU | C | 95 | 13.625 | 27.347 | −6.518 | 1.00 | 50.00 | C |
| ATOM | 3846 | O | LEU | C | 95 | 14.036 | 28.482 | −6.757 | 1.00 | 50.00 | O |
| ATOM | 3847 | CB | LEU | C | 95 | 11.688 | 26.729 | −5.013 | 1.00 | 50.00 | C |
| ATOM | 3848 | CG | LEU | C | 95 | 10.202 | 26.796 | −4.659 | 1.00 | 50.00 | C |
| ATOM | 3849 | CD1 | LEU | C | 95 | 9.203 | 25.934 | −5.423 | 1.00 | 50.00 | C |
| ATOM | 3850 | CD2 | LEU | C | 95 | 9.885 | 26.533 | −3.187 | 1.00 | 50.00 | C |
| ATOM | 3851 | N | LEU | C | 96 | 14.427 | 26.311 | −6.334 | 1.00 | 50.00 | N |
| ATOM | 3852 | CA | LEU | C | 96 | 15.886 | 26.302 | −6.436 | 1.00 | 50.00 | C |
| ATOM | 3853 | C | LEU | C | 96 | 16.479 | 26.619 | −7.806 | 1.00 | 50.00 | C |
| ATOM | 3854 | O | LEU | C | 96 | 17.543 | 27.235 | −7.850 | 1.00 | 50.00 | O |
| ATOM | 3855 | CB | LEU | C | 96 | 16.352 | 24.932 | −5.938 | 1.00 | 50.00 | C |
| ATOM | 3856 | CG | LEU | C | 96 | 16.375 | 24.615 | −4.441 | 1.00 | 50.00 | C |
| ATOM | 3857 | CD1 | LEU | C | 96 | 15.080 | 24.707 | −3.634 | 1.00 | 50.00 | C |
| ATOM | 3858 | CD2 | LEU | C | 96 | 16.931 | 23.206 | −4.239 | 1.00 | 50.00 | C |
| ATOM | 3859 | N | MET | C | 97 | 15.839 | 26.246 | −8.904 | 1.00 | 50.00 | N |
| ATOM | 3860 | CA | MET | C | 97 | 16.156 | 26.682 | −10.266 | 1.00 | 50.00 | C |
| ATOM | 3861 | C | MET | C | 97 | 16.344 | 28.176 | −10.503 | 1.00 | 50.00 | C |
| ATOM | 3862 | O | MET | C | 97 | 17.248 | 28.602 | −11.221 | 1.00 | 50.00 | O |
| ATOM | 3863 | CB | MET | C | 97 | 15.068 | 26.177 | −11.215 | 1.00 | 50.00 | C |
| ATOM | 3864 | CG | MET | C | 97 | 15.045 | 24.671 | −11.466 | 1.00 | 50.00 | C |
| ATOM | 3865 | SD | MET | C | 97 | 16.642 | 24.078 | −12.049 | 1.00 | 50.00 | S |
| ATOM | 3866 | CE | MET | C | 97 | 16.599 | 24.666 | −13.750 | 1.00 | 50.00 | C |
| ATOM | 3867 | N | GLN | C | 98 | 15.468 | 28.959 | −9.899 | 1.00 | 50.00 | N |
| ATOM | 3868 | CA | GLN | C | 98 | 15.461 | 30.418 | −9.955 | 1.00 | 50.00 | C |
| ATOM | 3869 | C | GLN | C | 98 | 16.328 | 31.037 | −8.865 | 1.00 | 50.00 | C |
| ATOM | 3870 | O | GLN | C | 98 | 16.957 | 30.419 | −8.007 | 1.00 | 50.00 | O |
| ATOM | 3871 | CB | GLN | C | 98 | 13.984 | 30.735 | −9.733 | 1.00 | 50.00 | C |
| ATOM | 3872 | CG | GLN | C | 98 | 12.844 | 30.497 | −10.720 | 1.00 | 50.00 | C |
| ATOM | 3873 | CD | GLN | C | 98 | 12.776 | 31.522 | −11.841 | 1.00 | 50.00 | C |
| ATOM | 3874 | OE1 | GLN | C | 98 | 13.543 | 32.479 | −11.936 | 1.00 | 50.00 | O |
| ATOM | 3875 | NE2 | GLN | C | 98 | 11.796 | 31.354 | −12.709 | 1.00 | 50.00 | N |
| ATOM | 3876 | N | LEU | D | 160 | −4.061 | −93.407 | 4.256 | 1.00 | 50.00 | N |
| ATOM | 3877 | CA | LEU | D | 160 | −2.785 | −93.331 | 4.984 | 1.00 | 50.00 | C |
| ATOM | 3878 | C | LEU | D | 160 | −1.748 | −92.606 | 4.126 | 1.00 | 50.00 | C |
| ATOM | 3879 | O | LEU | D | 160 | −1.355 | −91.502 | 4.501 | 1.00 | 50.00 | O |
| ATOM | 3880 | CB | LEU | D | 160 | −2.585 | −94.484 | 5.979 | 1.00 | 50.00 | C |
| ATOM | 3881 | CG | LEU | D | 160 | −2.197 | −95.961 | 5.830 | 1.00 | 50.00 | C |
| ATOM | 3882 | CD1 | LEU | D | 160 | −2.545 | −96.430 | 4.420 | 1.00 | 50.00 | C |
| ATOM | 3883 | CD2 | LEU | D | 160 | −0.960 | −96.736 | 6.292 | 1.00 | 50.00 | C |
| ATOM | 3884 | N | GLU | D | 161 | −1.304 | −93.117 | 2.987 | 1.00 | 50.00 | N |
| ATOM | 3885 | CA | GLU | D | 161 | −0.356 | −92.479 | 2.068 | 1.00 | 50.00 | C |
| ATOM | 3886 | C | GLU | D | 161 | −0.869 | −91.414 | 1.105 | 1.00 | 50.00 | C |
| ATOM | 3887 | O | GLU | D | 161 | −0.332 | −90.309 | 1.043 | 1.00 | 50.00 | O |
| ATOM | 3888 | CB | GLU | D | 161 | 0.328 | −93.516 | 1.174 | 1.00 | 50.00 | C |
| ATOM | 3889 | CG | GLU | D | 161 | 1.303 | −93.119 | 0.062 | 1.00 | 50.00 | C |
| ATOM | 3890 | CD | GLU | D | 161 | 2.509 | −92.209 | 0.251 | 1.00 | 50.00 | C |
| ATOM | 3891 | OE1 | GLU | D | 161 | 3.625 | −92.721 | 0.488 | 1.00 | 50.00 | O |
| ATOM | 3892 | OE2 | GLU | D | 161 | 2.381 | −90.975 | 0.093 | 1.00 | 50.00 | O |
| ATOM | 3893 | N | GLY | D | 162 | −1.854 | −91.730 | 0.279 | 1.00 | 50.00 | N |
| ATOM | 3894 | CA | GLY | D | 162 | −2.661 | −90.728 | −0.422 | 1.00 | 50.00 | C |
| ATOM | 3895 | C | GLY | D | 162 | −3.117 | −89.473 | 0.311 | 1.00 | 50.00 | C |
| ATOM | 3896 | O | GLY | D | 162 | −3.383 | −88.402 | −0.231 | 1.00 | 50.00 | O |
| ATOM | 3897 | N | GLU | D | 163 | −3.189 | −89.701 | 1.608 | 1.00 | 50.00 | N |
| ATOM | 3898 | CA | GLU | D | 163 | −3.436 | −88.731 | 2.663 | 1.00 | 50.00 | C |
| ATOM | 3899 | C | GLU | D | 163 | −2.208 | −88.005 | 3.198 | 1.00 | 50.00 | C |
| ATOM | 3900 | O | GLU | D | 163 | −2.296 | −86.798 | 3.422 | 1.00 | 50.00 | O |
| ATOM | 3901 | CB | GLU | D | 163 | −4.104 | −89.787 | 3.542 | 1.00 | 50.00 | C |
| ATOM | 3902 | CG | GLU | D | 163 | −5.501 | −90.387 | 3.385 | 1.00 | 50.00 | C |
| ATOM | 3903 | CD | GLU | D | 163 | −6.566 | −89.315 | 3.218 | 1.00 | 50.00 | C |
| ATOM | 3904 | OE1 | GLU | D | 163 | −6.604 | −88.784 | 2.089 | 1.00 | 50.00 | O |
| ATOM | 3905 | OE2 | GLU | D | 163 | −7.281 | −88.932 | 4.170 | 1.00 | 50.00 | O |
| ATOM | 3906 | N | VAL | D | 164 | −1.051 | −88.627 | 3.348 | 1.00 | 50.00 | N |
| ATOM | 3907 | CA | VAL | D | 164 | 0.200 | −87.908 | 3.576 | 1.00 | 50.00 | C |
| ATOM | 3908 | C | VAL | D | 164 | 0.742 | −86.839 | 2.630 | 1.00 | 50.00 | C |
| ATOM | 3909 | O | VAL | D | 164 | 1.649 | −86.061 | 2.926 | 1.00 | 50.00 | O |
| ATOM | 3910 | CB | VAL | D | 164 | 1.199 | −89.007 | 3.926 | 1.00 | 50.00 | C |
| ATOM | 3911 | CG1 | VAL | D | 164 | 2.017 | −89.589 | 2.777 | 1.00 | 50.00 | C |
| ATOM | 3912 | CG2 | VAL | D | 164 | 2.200 | −88.314 | 4.837 | 1.00 | 50.00 | C |
| ATOM | 3913 | N | ASN | D | 165 | 0.221 | −86.862 | 1.418 | 1.00 | 50.00 | N |
| ATOM | 3914 | CA | ASN | D | 165 | 0.436 | −85.837 | 0.399 | 1.00 | 50.00 | C |
| ATOM | 3915 | C | ASN | D | 165 | −0.649 | −84.768 | 0.324 | 1.00 | 50.00 | C |
| ATOM | 3916 | O | ASN | D | 165 | −0.381 | −83.726 | −0.275 | 1.00 | 50.00 | O |
| ATOM | 3917 | CB | ASN | D | 165 | 0.685 | −86.631 | −0.883 | 1.00 | 50.00 | C |
| ATOM | 3918 | CG | ASN | D | 165 | −0.521 | −87.327 | −1.502 | 1.00 | 50.00 | C |
| ATOM | 3919 | OD1 | ASN | D | 165 | −1.598 | −86.757 | −1.664 | 1.00 | 50.00 | O |
| ATOM | 3920 | ND2 | ASN | D | 165 | −0.397 | −88.601 | −1.825 | 1.00 | 50.00 | N |
| ATOM | 3921 | N | LYS | D | 166 | −1.838 | −84.972 | 0.873 | 1.00 | 50.00 | N |
| ATOM | 3922 | CA | LYS | D | 166 | −2.926 | −83.993 | 0.901 | 1.00 | 50.00 | C |
| ATOM | 3923 | C | LYS | D | 166 | −2.485 | −82.693 | 1.552 | 1.00 | 50.00 | C |
| ATOM | 3924 | O | LYS | D | 166 | −2.237 | −81.702 | 0.871 | 1.00 | 50.00 | O |
| ATOM | 3925 | CB | LYS | D | 166 | −4.192 | −84.491 | 1.599 | 1.00 | 50.00 | C |
| ATOM | 3926 | CG | LYS | D | 166 | −4.939 | −85.504 | 0.739 | 1.00 | 50.00 | C |
| ATOM | 3927 | CD | LYS | D | 166 | −6.370 | −85.736 | 1.216 | 1.00 | 50.00 | C |
| ATOM | 3928 | CE | LYS | D | 166 | −7.050 | −86.665 | 0.213 | 1.00 | 50.00 | C |
| ATOM | 3929 | NZ | LYS | D | 166 | −8.350 | −87.108 | 0.735 | 1.00 | 50.00 | N |
| ATOM | 3930 | N | ILE | D | 167 | −2.252 | −82.786 | 2.846 | 1.00 | 50.00 | N |
| ATOM | 3931 | CA | ILE | D | 167 | −1.355 | −81.953 | 3.643 | 1.00 | 50.00 | C |
| ATOM | 3932 | C | ILE | D | 167 | −0.082 | −81.305 | 3.135 | 1.00 | 50.00 | C |
| ATOM | 3933 | O | ILE | D | 167 | 0.323 | −80.260 | 3.640 | 1.00 | 50.00 | O |
| ATOM | 3934 | CB | ILE | D | 167 | −1.040 | −82.765 | 4.898 | 1.00 | 50.00 | C |
| ATOM | 3935 | CG1 | ILE | D | 167 | −0.575 | −84.207 | 4.645 | 1.00 | 50.00 | C |
| ATOM | 3936 | CG2 | ILE | D | 167 | −2.330 | −82.390 | 5.626 | 1.00 | 50.00 | C |
| ATOM | 3937 | CD1 | ILE | D | 167 | −0.130 | −85.242 | 5.679 | 1.00 | 50.00 | C |
| ATOM | 3938 | N | LYS | D | 168 | 0.560 | −81.968 | 2.195 | 1.00 | 50.00 | N |
| ATOM | 3939 | CA | LYS | D | 168 | 1.783 | −81.461 | 1.581 | 1.00 | 50.00 | C |
| ATOM | 3940 | C | LYS | D | 168 | 1.450 | −80.313 | 0.637 | 1.00 | 50.00 | C |
| ATOM | 3941 | O | LYS | D | 168 | 1.716 | −79.156 | 0.963 | 1.00 | 50.00 | O |
| ATOM | 3942 | CB | LYS | D | 168 | 2.439 | −82.788 | 1.199 | 1.00 | 50.00 | C |
| ATOM | 3943 | CG | LYS | D | 168 | 3.933 | −82.582 | 0.992 | 1.00 | 50.00 | C |
| ATOM | 3944 | CD | LYS | D | 168 | 4.770 | −83.754 | 0.488 | 1.00 | 50.00 | C |
| ATOM | 3945 | CE | LYS | D | 168 | 5.902 | −83.224 | −0.389 | 1.00 | 50.00 | C |
| ATOM | 3946 | NZ | LYS | D | 168 | 5.351 | −82.593 | −1.599 | 1.00 | 50.00 | N |
| ATOM | 3947 | N | SER | D | 169 | 0.693 | −80.624 | −0.400 | 1.00 | 50.00 | N |
| ATOM | 3948 | CA | SER | D | 169 | −0.151 | −79.668 | −1.118 | 1.00 | 50.00 | C |
| ATOM | 3949 | C | SER | D | 169 | −0.921 | −78.616 | −0.327 | 1.00 | 50.00 | C |
| ATOM | 3950 | O | SER | D | 169 | −0.978 | −77.448 | −0.710 | 1.00 | 50.00 | O |
| ATOM | 3951 | CB | SER | D | 169 | −1.167 | −80.507 | −1.893 | 1.00 | 50.00 | C |
| ATOM | 3952 | OG | SER | D | 169 | −0.551 | −81.357 | −2.850 | 1.00 | 50.00 | O |
| ATOM | 3953 | N | ALA | D | 170 | −1.518 | −79.039 | 0.773 | 1.00 | 50.00 | N |
| ATOM | 3954 | CA | ALA | D | 170 | −2.402 | −78.228 | 1.604 | 1.00 | 50.00 | C |
| ATOM | 3955 | C | ALA | D | 170 | −1.556 | −77.204 | 2.353 | 1.00 | 50.00 | C |
| ATOM | 3956 | O | ALA | D | 170 | −1.692 | −76.011 | 2.086 | 1.00 | 50.00 | O |
| ATOM | 3957 | CB | ALA | D | 170 | −3.289 | −79.257 | 2.307 | 1.00 | 50.00 | C |
| ATOM | 3958 | N | LEU | D | 171 | −0.658 | −77.565 | 3.253 | 1.00 | 50.00 | N |
| ATOM | 3959 | CA | LEU | D | 171 | 0.340 | −76.661 | 3.825 | 1.00 | 50.00 | C |
| ATOM | 3960 | C | LEU | D | 171 | 1.080 | −75.693 | 2.909 | 1.00 | 50.00 | C |
| ATOM | 3961 | O | LEU | D | 171 | 1.251 | −74.534 | 3.283 | 1.00 | 50.00 | O |
| ATOM | 3962 | CB | LEU | D | 171 | 1.324 | −77.678 | 4.394 | 1.00 | 50.00 | C |
| ATOM | 3963 | CG | LEU | D | 171 | 2.238 | −77.305 | 5.555 | 1.00 | 50.00 | C |
| ATOM | 3964 | CD1 | LEU | D | 171 | 3.406 | −78.258 | 5.326 | 1.00 | 50.00 | C |
| ATOM | 3965 | CD2 | LEU | D | 171 | 2.644 | −75.836 | 5.558 | 1.00 | 50.00 | C |
| ATOM | 3966 | N | LEU | D | 172 | 1.515 | −76.099 | 1.728 | 1.00 | 50.00 | N |
| ATOM | 3967 | CA | LEU | D | 172 | 1.944 | −75.178 | 0.676 | 1.00 | 50.00 | C |
| ATOM | 3968 | C | LEU | D | 172 | 1.055 | −73.982 | 0.360 | 1.00 | 50.00 | C |
| ATOM | 3969 | O | LEU | D | 172 | 1.535 | −72.854 | 0.251 | 1.00 | 50.00 | O |
| ATOM | 3970 | CB | LEU | D | 172 | 2.090 | −75.919 | −0.653 | 1.00 | 50.00 | C |
| ATOM | 3971 | CG | LEU | D | 172 | 3.047 | −75.290 | −1.667 | 1.00 | 50.00 | C |
| ATOM | 3972 | CD1 | LEU | D | 172 | 2.274 | −74.503 | −2.720 | 1.00 | 50.00 | C |
| ATOM | 3973 | CD2 | LEU | D | 172 | 4.176 | −74.384 | −1.170 | 1.00 | 50.00 | C |
| ATOM | 3974 | N | SER | D | 173 | −0.222 | −74.260 | 0.158 | 1.00 | 50.00 | N |
| ATOM | 3975 | CA | SER | D | 173 | −1.266 | −73.248 | 0.021 | 1.00 | 50.00 | C |
| ATOM | 3976 | C | SER | D | 173 | −1.364 | −72.268 | 1.182 | 1.00 | 50.00 | C |
| ATOM | 3977 | O | SER | D | 173 | −1.467 | −71.074 | 0.904 | 1.00 | 50.00 | O |
| ATOM | 3978 | CB | SER | D | 173 | −2.600 | −73.960 | −0.192 | 1.00 | 50.00 | C |
| ATOM | 3979 | OG | SER | D | 173 | −2.710 | −74.491 | −1.504 | 1.00 | 50.00 | O |
| ATOM | 3980 | N | THR | D | 174 | −1.219 | −72.711 | 2.421 | 1.00 | 50.00 | N |
| ATOM | 3981 | CA | THR | D | 174 | −0.801 | −71.821 | 3.503 | 1.00 | 50.00 | C |
| ATOM | 3982 | C | THR | D | 174 | 0.481 | −71.038 | 3.245 | 1.00 | 50.00 | C |
| ATOM | 3983 | O | THR | D | 174 | 0.510 | −69.842 | 3.532 | 1.00 | 50.00 | O |
| ATOM | 3984 | CB | THR | D | 174 | −0.542 | −72.477 | 4.857 | 1.00 | 50.00 | C |
| ATOM | 3985 | OG1 | THR | D | 174 | −1.579 | −73.246 | 5.435 | 1.00 | 50.00 | O |
| ATOM | 3986 | CG2 | THR | O | 174 | −0.497 | −71.381 | 5.914 | 1.00 | 50.00 | C |
| ATOM | 3987 | N | ASN | D | 175 | 1.594 | −71.659 | 2.896 | 1.00 | 50.00 | N |
| ATOM | 3988 | CA | ASN | D | 175 | 2.856 | −70.939 | 2.731 | 1.00 | 50.00 | C |
| ATOM | 3989 | C | ASN | D | 175 | 2.786 | −69.704 | 1.843 | 1.00 | 50.00 | C |
| ATOM | 3990 | O | ASN | D | 175 | 2.988 | −68.583 | 2.307 | 1.00 | 50.00 | O |
| ATOM | 3991 | CB | ASN | D | 175 | 3.955 | −71.908 | 2.298 | 1.00 | 50.00 | C |
| ATOM | 3992 | CG | ASN | D | 175 | 4.547 | −72.627 | 3.496 | 1.00 | 50.00 | C |
| ATOM | 3993 | OD1 | ASN | D | 175 | 5.322 | −72.132 | 4.313 | 1.00 | 50.00 | O |
| ATOM | 3994 | ND2 | ASN | D | 175 | 4.153 | −73.882 | 3.546 | 1.00 | 50.00 | N |
| ATOM | 3995 | N | LYS | D | 176 | 2.334 | −69.920 | 0.621 | 1.00 | 50.00 | N |
| ATOM | 3996 | CA | LYS | D | 176 | 1.831 | −68.876 | −0.270 | 1.00 | 50.00 | C |
| ATOM | 3997 | C | LYS | D | 176 | 0.876 | −67.833 | 0.297 | 1.00 | 50.00 | C |
| ATOM | 3998 | O | LYS | D | 176 | 1.047 | −66.647 | 0.017 | 1.00 | 50.00 | O |
| ATOM | 3999 | CB | LYS | D | 176 | 1.144 | −69.597 | −1.428 | 1.00 | 50.00 | C |
| ATOM | 4000 | CG | LYS | D | 176 | 2.088 | −70.440 | −2.282 | 1.00 | 50.00 | C |
| ATOM | 4001 | CD | LYS | D | 176 | 1.350 | −70.929 | −3.525 | 1.00 | 50.00 | C |
| ATOM | 4002 | CE | LYS | O | 176 | 2.276 | −71.702 | −4.459 | 1.00 | 50.00 | C |
| ATOM | 4003 | NZ | LYS | D | 176 | 1.475 | −72.599 | −5.305 | 1.00 | 50.00 | N |
| ATOM | 4004 | N | ALA | D | 177 | −0.094 | −68.247 | 1.095 | 1.00 | 50.00 | N |
| ATOM | 4005 | CA | ALA | D | 177 | −0.938 | −67.362 | 1.895 | 1.00 | 50.00 | C |
| ATOM | 4006 | C | ALA | D | 177 | −0.246 | −66.411 | 2.864 | 1.00 | 50.00 | C |
| ATOM | 4007 | O | ALA | D | 177 | −0.607 | −65.235 | 2.905 | 1.00 | 50.00 | O |
| ATOM | 4008 | CB | ALA | D | 177 | −1.912 | −68.339 | 2.548 | 1.00 | 50.00 | C |
| ATOM | 4009 | N | VAL | D | 178 | 0.785 | −66.850 | 3.568 | 1.00 | 50.00 | N |
| ATOM | 4010 | CA | VAL | D | 178 | 1.722 | −65.979 | 4.277 | 1.00 | 50.00 | C |
| ATOM | 4011 | C | VAL | D | 178 | 2.581 | −65.097 | 3.375 | 1.00 | 50.00 | C |
| ATOM | 4012 | O | VAL | D | 178 | 2.666 | −63.921 | 3.724 | 1.00 | 50.00 | O |
| ATOM | 4013 | CB | VAL | D | 178 | 2.606 | −66.764 | 5.246 | 1.00 | 50.00 | C |
| ATOM | 4014 | CG1 | VAL | D | 178 | 3.375 | −65.795 | 6.141 | 1.00 | 50.00 | C |
| ATOM | 4015 | CG2 | VAL | D | 178 | 1.917 | −67.764 | 6.171 | 1.00 | 50.00 | C |
| ATOM | 4016 | N | VAL | D | 179 | 3.243 | −65.546 | 2.319 | 1.00 | 50.00 | N |
| ATOM | 4017 | CA | VAL | D | 179 | 3.915 | −64.688 | 1.338 | 1.00 | 50.00 | C |
| ATOM | 4018 | C | VAL | D | 179 | 3.074 | −63.523 | 0.834 | 1.00 | 50.00 | C |
| ATOM | 4019 | O | VAL | D | 179 | 3.528 | −62.381 | 0.877 | 1.00 | 50.00 | O |
| ATOM | 4020 | CB | VAL | D | 179 | 4.420 | −65.426 | 0.093 | 1.00 | 50.00 | C |
| ATOM | 4021 | CG1 | VAL | D | 179 | 4.821 | −64.743 | −1.214 | 1.00 | 50.00 | C |
| ATOM | 4022 | CG2 | VAL | D | 179 | 5.645 | −66.253 | 0.460 | 1.00 | 50.00 | C |
| ATOM | 4023 | N | SER | D | 180 | 1.905 | −63.857 | 0.314 | 1.00 | 50.00 | N |
| ATOM | 4024 | CA | SER | D | 180 | 0.915 | −62.922 | −0.217 | 1.00 | 50.00 | C |
| ATOM | 4025 | C | SER | D | 180 | 0.563 | −61.772 | 0.717 | 1.00 | 50.00 | C |
| ATOM | 4026 | O | SER | D | 180 | 0.695 | −60.602 | 0.360 | 1.00 | 50.00 | O |
| ATOM | 4027 | CB | SER | D | 180 | −0.329 | −63.747 | −0.547 | 1.00 | 50.00 | C |
| ATOM | 4028 | OG | SER | D | 180 | −0.130 | −64.584 | −1.678 | 1.00 | 50.00 | O |
| ATOM | 4029 | N | LEU | D | 181 | 0.214 | −62.136 | 1.938 | 1.00 | 50.00 | N |
| ATOM | 4030 | CA | LEU | D | 181 | 0.134 | −61.231 | 3.082 | 1.00 | 50.00 | C |
| ATOM | 4031 | C | LEU | D | 181 | 1.439 | −60.510 | 3.398 | 1.00 | 50.00 | C |
| ATOM | 4032 | O | LEU | D | 181 | 1.374 | −59.292 | 3.547 | 1.00 | 50.00 | O |
| ATOM | 4033 | CB | LEU | D | 181 | −0.475 | −62.136 | 4.153 | 1.00 | 50.00 | C |
| ATOM | 4034 | CG | LEU | D | 181 | −1.153 | −61.478 | 5.352 | 1.00 | 50.00 | C |
| ATOM | 4035 | CD1 | LEU | D | 181 | −2.190 | −60.405 | 5.031 | 1.00 | 50.00 | C |
| ATOM | 4036 | CD2 | LEU | D | 181 | −1.774 | −62.638 | 6.125 | 1.00 | 50.00 | C |
| ATOM | 4037 | N | SER | D | 182 | 2.592 | −61.159 | 3.453 | 1.00 | 50.00 | N |
| ATOM | 4038 | CA | SER | D | 182 | 3.901 | −60.535 | 3.655 | 1.00 | 50.00 | C |
| ATOM | 4039 | C | SER | D | 182 | 4.272 | −59.360 | 2.763 | 1.00 | 50.00 | C |
| ATOM | 4040 | O | SER | D | 182 | 4.550 | −58.262 | 3.243 | 1.00 | 50.00 | O |
| ATOM | 4041 | CB | SER | D | 182 | 5.015 | −61.543 | 3.933 | 1.00 | 50.00 | C |
| ATOM | 4042 | OG | SER | D | 182 | 4.649 | −62.331 | 5.054 | 1.00 | 50.00 | O |
| ATOM | 4043 | N | ASN | D | 183 | 4.139 | −59.594 | 1.471 | 1.00 | 50.00 | N |
| ATOM | 4044 | CA | ASN | D | 183 | 4.055 | −58.556 | 0.444 | 1.00 | 50.00 | C |
| ATOM | 4045 | C | ASN | D | 183 | 2.999 | −57.478 | 0.667 | 1.00 | 50.00 | C |
| ATOM | 4046 | O | ASN | D | 183 | 3.343 | −56.302 | 0.553 | 1.00 | 50.00 | O |
| ATOM | 4047 | CB | ASN | D | 183 | 3.895 | −59.289 | −0.889 | 1.00 | 50.00 | C |
| ATOM | 4048 | CG | ASN | D | 183 | 4.999 | −60.259 | −1.290 | 1.00 | 50.00 | C |
| ATOM | 4049 | OD1 | ASN | D | 183 | 6.178 | −60.056 | −1.004 | 1.00 | 50.00 | O |
| ATOM | 4050 | ND2 | ASN | D | 183 | 4.648 | −61.365 | −1.919 | 1.00 | 50.00 | N |
| ATOM | 4051 | N | GLY | D | 184 | 1.771 | −57.807 | 1.036 | 1.00 | 50.00 | N |
| ATOM | 4052 | CA | GLY | D | 184 | 0.758 | −56.856 | 1.496 | 1.00 | 50.00 | C |
| ATOM | 4053 | C | GLY | D | 184 | 1.092 | −55.899 | 2.634 | 1.00 | 50.00 | C |
| ATOM | 4054 | O | GLY | D | 184 | 0.877 | −54.694 | 2.515 | 1.00 | 50.00 | O |
| ATOM | 4055 | N | VAL | D | 185 | 1.656 | −56.430 | 3.706 | 1.00 | 50.00 | N |
| ATOM | 4056 | CA | VAL | D | 185 | 2.289 | −55.691 | 4.801 | 1.00 | 50.00 | C |
| ATOM | 4057 | C | VAL | D | 185 | 3.466 | −54.832 | 4.353 | 1.00 | 50.00 | C |
| ATOM | 4058 | O | VAL | D | 185 | 3.566 | −53.709 | 4.843 | 1.00 | 50.00 | O |
| ATOM | 4059 | CB | VAL | D | 185 | 2.662 | −56.635 | 5.947 | 1.00 | 50.00 | C |
| ATOM | 4060 | CG1 | VAL | D | 185 | 3.172 | −55.961 | 7.222 | 1.00 | 50.00 | C |
| ATOM | 4061 | CG2 | VAL | D | 185 | 1.493 | −57.544 | 6.325 | 1.00 | 50.00 | C |
| ATOM | 4062 | N | SER | D | 186 | 4.322 | −55.290 | 3.454 | 1.00 | 50.00 | N |
| ATOM | 4063 | CA | SER | D | 186 | 5.471 | −54.557 | 2.918 | 1.00 | 50.00 | C |
| ATOM | 4064 | C | SER | D | 186 | 5.182 | −53.239 | 2.219 | 1.00 | 50.00 | C |
| ATOM | 4065 | O | SER | D | 186 | 5.838 | −52.230 | 2.473 | 1.00 | 50.00 | O |
| ATOM | 4066 | CB | SER | D | 186 | 6.255 | −55.435 | 1.940 | 1.00 | 50.00 | C |
| ATOM | 4067 | OG | SER | D | 186 | 7.636 | −55.105 | 1.943 | 1.00 | 50.00 | O |
| ATOM | 4068 | N | VAL | D | 187 | 4.192 | −53.310 | 1.347 | 1.00 | 50.00 | N |
| ATOM | 4069 | CA | VAL | D | 187 | 3.610 | −52.156 | 0.669 | 1.00 | 50.00 | C |
| ATOM | 4070 | C | VAL | D | 187 | 2.885 | −51.246 | 1.653 | 1.00 | 50.00 | C |
| ATOM | 4071 | O | VAL | D | 187 | 3.146 | −50.045 | 1.616 | 1.00 | 50.00 | O |
| ATOM | 4072 | CB | VAL | D | 187 | 2.765 | −52.636 | −0.511 | 1.00 | 50.00 | C |
| ATOM | 4073 | CG1 | VAL | D | 187 | 2.243 | −51.429 | −1.288 | 1.00 | 50.00 | C |
| ATOM | 4074 | CG2 | VAL | D | 187 | 3.477 | −53.566 | −1.494 | 1.00 | 50.00 | C |
| ATOM | 4075 | N | LEU | D | 188 | 2.048 | −51.744 | 2.548 | 1.00 | 50.00 | N |
| ATOM | 4076 | CA | LEU | D | 188 | 1.535 | −50.969 | 3.678 | 1.00 | 50.00 | C |
| ATOM | 4077 | C | LEU | D | 188 | 2.562 | −50.290 | 4.580 | 1.00 | 50.00 | C |
| ATOM | 4078 | O | LEU | D | 188 | 2.322 | −49.172 | 5.034 | 1.00 | 50.00 | O |
| ATOM | 4079 | CB | LEU | D | 188 | 0.567 | −51.914 | 4.388 | 1.00 | 50.00 | C |
| ATOM | 4080 | CG | LEU | D | 188 | −0.362 | −51.302 | 5.436 | 1.00 | 50.00 | C |
| ATOM | 4081 | CD1 | LEU | D | 188 | −1.284 | −50.202 | 4.914 | 1.00 | 50.00 | C |
| ATOM | 4082 | CD2 | LEU | D | 188 | −1.194 | −52.467 | 5.966 | 1.00 | 50.00 | C |
| ATOM | 4083 | N | THR | D | 189 | 3.706 | −50.919 | 4.801 | 1.00 | 50.00 | N |
| ATOM | 4084 | CA | THR | D | 189 | 4.912 | −50.342 | 5.393 | 1.00 | 50.00 | C |
| ATOM | 4085 | C | THR | D | 189 | 5.522 | −49.247 | 4.534 | 1.00 | 50.00 | C |
| ATOM | 4086 | O | THR | D | 189 | 5.503 | −48.127 | 5.041 | 1.00 | 50.00 | O |
| ATOM | 4087 | CB | THR | D | 189 | 5.941 | −51.386 | 5.825 | 1.00 | 50.00 | C |
| ATOM | 4088 | OG1 | THR | D | 189 | 5.261 | −52.464 | 6.450 | 1.00 | 50.00 | O |
| ATOM | 4089 | CG2 | THR | D | 189 | 6.988 | −50.843 | 6.795 | 1.00 | 50.00 | C |
| ATOM | 4090 | N | SER | D | 190 | 5.942 | −49.521 | 3.310 | 1.00 | 50.00 | N |
| ATOM | 4091 | CA | SER | D | 190 | 6.159 | −48.529 | 2.254 | 1.00 | 50.00 | C |
| ATOM | 4092 | C | SER | D | 190 | 5.256 | −47.298 | 2.241 | 1.00 | 50.00 | C |
| ATOM | 4093 | O | SER | D | 190 | 5.722 | −46.185 | 2.005 | 1.00 | 50.00 | O |
| ATOM | 4094 | CB | SER | D | 190 | 6.417 | −49.247 | 0.930 | 1.00 | 50.00 | C |
| ATOM | 4095 | OG | SER | D | 190 | 5.257 | −49.349 | 0.119 | 1.00 | 50.00 | O |
| ATOM | 4096 | N | LYS | D | 191 | 3.988 | −47.480 | 2.573 | 1.00 | 50.00 | N |
| ATOM | 4097 | CA | LYS | D | 191 | 2.994 | −46.432 | 2.798 | 1.00 | 50.00 | C |
| ATOM | 4098 | C | LYS | D | 191 | 3.128 | −45.669 | 4.112 | 1.00 | 50.00 | C |
| ATOM | 4099 | O | LYS | D | 191 | 3.222 | −44.444 | 4.043 | 1.00 | 50.00 | O |
| ATOM | 4100 | CB | LYS | D | 191 | 1.624 | −47.034 | 2.484 | 1.00 | 50.00 | C |
| ATOM | 4101 | CG | LYS | D | 191 | 1.319 | −47.399 | 1.030 | 1.00 | 50.00 | C |
| ATOM | 4102 | CD | LYS | D | 191 | 1.786 | −46.449 | −0.073 | 1.00 | 50.00 | C |
| ATOM | 4103 | CE | LYS | D | 191 | 3.170 | −46.704 | −0.668 | 1.00 | 50.00 | C |
| ATOM | 4104 | NZ | LYS | D | 191 | 3.550 | −45.660 | −1.631 | 1.00 | 50.00 | N |
| ATOM | 4105 | N | VAL | D | 192 | 3.260 | −46.291 | 5.273 | 1.00 | 50.00 | N |
| ATOM | 4106 | CA | VAL | D | 192 | 3.939 | −45.744 | 6.451 | 1.00 | 50.00 | C |
| ATOM | 4107 | C | VAL | D | 192 | 5.236 | −44.982 | 6.170 | 1.00 | 50.00 | C |
| ATOM | 4108 | O | VAL | D | 192 | 5.247 | −43.810 | 6.544 | 1.00 | 50.00 | O |
| ATOM | 4109 | CB | VAL | D | 192 | 3.835 | −46.772 | 7.585 | 1.00 | 50.00 | C |
| ATOM | 4110 | CG1 | VAL | D | 192 | 5.034 | −47.069 | 8.483 | 1.00 | 50.00 | C |
| ATOM | 4111 | CG2 | VAL | D | 192 | 2.634 | −46.593 | 8.515 | 1.00 | 50.00 | C |
| ATOM | 4112 | N | LEU | D | 193 | 6.245 | −45.458 | 5.453 | 1.00 | 50.00 | N |
| ATOM | 4113 | CA | LEU | D | 193 | 7.435 | −44.714 | 5.020 | 1.00 | 50.00 | C |
| ATOM | 4114 | C | LEU | D | 193 | 7.151 | −43.339 | 4.424 | 1.00 | 50.00 | C |
| ATOM | 4115 | O | LEU | D | 193 | 7.644 | −42.333 | 4.932 | 1.00 | 50.00 | O |
| ATOM | 4116 | CB | LEU | D | 193 | 8.401 | −45.366 | 4.023 | 1.00 | 50.00 | C |
| ATOM | 4117 | CG | LEU | D | 193 | 9.304 | −46.565 | 4.316 | 1.00 | 50.00 | C |
| ATOM | 4118 | CD1 | LEU | D | 193 | 8.512 | −47.851 | 4.536 | 1.00 | 50.00 | C |
| ATOM | 4119 | CD2 | LEU | D | 193 | 10.262 | −46.933 | 3.184 | 1.00 | 50.00 | C |
| ATOM | 4120 | N | ASP | D | 194 | 6.400 | −43.325 | 3.335 | 1.00 | 50.00 | N |
| ATOM | 4121 | CA | ASP | D | 194 | 6.147 | −42.143 | 2.513 | 1.00 | 50.00 | C |
| ATOM | 4122 | C | ASP | D | 194 | 5.241 | −41.137 | 3.204 | 1.00 | 50.00 | C |
| ATOM | 4123 | O | ASP | D | 194 | 5.646 | −39.984 | 3.337 | 1.00 | 50.00 | O |
| ATOM | 4124 | CB | ASP | D | 194 | 5.505 | −42.514 | 1.176 | 1.00 | 50.00 | C |
| ATOM | 4125 | CG | ASP | D | 194 | 6.453 | −43.156 | 0.176 | 1.00 | 50.00 | C |
| ATOM | 4126 | OD1 | ASP | D | 194 | 7.482 | −42.534 | −0.164 | 1.00 | 50.00 | O |
| ATOM | 4127 | OD2 | ASP | D | 194 | 6.173 | −44.288 | −0.275 | 1.00 | 50.00 | O |
| ATOM | 4128 | N | LEU | D | 195 | 4.076 | −41.569 | 3.659 | 1.00 | 50.00 | N |
| ATOM | 4129 | CA | LEU | D | 195 | 3.194 | −40.778 | 4.512 | 1.00 | 50.00 | C |
| ATOM | 4130 | C | LEU | D | 195 | 3.918 | −40.063 | 5.647 | 1.00 | 50.00 | C |
| ATOM | 4131 | O | LEU | D | 195 | 3.889 | −38.834 | 5.679 | 1.00 | 50.00 | O |
| ATOM | 4132 | CB | LEU | D | 195 | 2.038 | −41.712 | 4.873 | 1.00 | 50.00 | C |
| ATOM | 4133 | CG | LEU | D | 195 | 0.787 | −41.018 | 5.405 | 1.00 | 50.00 | C |
| ATOM | 4134 | CD1 | LEU | D | 195 | 1.059 | −40.630 | 6.853 | 1.00 | 50.00 | C |
| ATOM | 4135 | CD2 | LEU | D | 195 | 0.254 | −39.838 | 4.597 | 1.00 | 50.00 | C |
| ATOM | 4136 | N | LYS | D | 196 | 4.612 | −40.760 | 6.532 | 1.00 | 50.00 | N |
| ATOM | 4137 | CA | LYS | D | 196 | 5.455 | −40.149 | 7.559 | 1.00 | 50.00 | C |
| ATOM | 4138 | C | LYS | D | 196 | 6.386 | −39.013 | 7.155 | 1.00 | 50.00 | C |
| ATOM | 4139 | O | LYS | D | 196 | 6.210 | −37.886 | 7.616 | 1.00 | 50.00 | O |
| ATOM | 4140 | CB | LYS | D | 196 | 6.232 | −41.270 | 8.246 | 1.00 | 50.00 | C |
| ATOM | 4141 | CG | LYS | D | 196 | 6.841 | −40.851 | 9.584 | 1.00 | 50.00 | C |
| ATOM | 4142 | CD | LYS | D | 196 | 5.828 | −41.056 | 10.708 | 1.00 | 50.00 | C |
| ATOM | 4143 | CE | LYS | D | 196 | 6.328 | −41.302 | 12.127 | 1.00 | 50.00 | C |
| ATOM | 4144 | NZ | LYS | D | 196 | 5.319 | −41.092 | 13.174 | 1.00 | 50.00 | N |
| ATOM | 4145 | N | ASN | D | 197 | 7.342 | −39.330 | 6.298 | 1.00 | 50.00 | N |
| ATOM | 4146 | CA | ASN | D | 197 | 8.251 | −38.363 | 5.686 | 1.00 | 50.00 | C |
| ATOM | 4147 | C | ASN | D | 197 | 7.587 | −37.253 | 4.880 | 1.00 | 50.00 | C |
| ATOM | 4148 | O | ASN | D | 197 | 8.086 | −36.130 | 4.919 | 1.00 | 50.00 | O |
| ATOM | 4149 | CB | ASN | D | 197 | 9.270 | −39.117 | 4.830 | 1.00 | 50.00 | C |
| ATOM | 4150 | CG | ASN | D | 197 | 10.250 | −40.067 | 5.507 | 1.00 | 50.00 | C |
| ATOM | 4151 | OD1 | ASN | D | 197 | 10.460 | −41.186 | 5.041 | 1.00 | 50.00 | O |
| ATOM | 4152 | ND2 | ASN | D | 197 | 10.899 | −39.672 | 6.587 | 1.00 | 50.00 | N |
| ATOM | 4153 | N | TYR | D | 198 | 6.462 | −37.480 | 4.221 | 1.00 | 50.00 | N |
| ATOM | 4154 | CA | TYR | D | 198 | 5.577 | −36.424 | 3.732 | 1.00 | 50.00 | C |
| ATOM | 4155 | C | TYR | D | 198 | 5.177 | −35.419 | 4.804 | 1.00 | 50.00 | C |
| ATOM | 4156 | O | TYR | D | 198 | 5.461 | −34.238 | 4.617 | 1.00 | 50.00 | O |
| ATOM | 4157 | CB | TYR | D | 198 | 4.344 | −36.978 | 3.016 | 1.00 | 50.00 | C |
| ATOM | 4158 | CG | TYR | D | 198 | 3.524 | −35.881 | 2.372 | 1.00 | 50.00 | C |
| ATOM | 4159 | CD1 | TYR | D | 198 | 3.910 | −35.393 | 1.175 | 1.00 | 50.00 | C |
| ATOM | 4160 | CD2 | TYR | D | 198 | 2.486 | −35.310 | 3.019 | 1.00 | 50.00 | C |
| ATOM | 4161 | CE1 | TYR | D | 198 | 3.223 | −34.387 | 0.597 | 1.00 | 50.00 | C |
| ATOM | 4162 | CE2 | TYR | D | 198 | 1.781 | −34.320 | 2.437 | 1.00 | 50.00 | C |
| ATOM | 4163 | CZ | TYR | D | 198 | 2.135 | −33.882 | 1.213 | 1.00 | 50.00 | C |
| ATOM | 4164 | OH | TYR | D | 198 | 1.266 | −33.102 | 0.497 | 1.00 | 50.00 | O |
| ATOM | 4165 | N | ILE | D | 199 | 4.512 | −35.848 | 5.864 | 1.00 | 50.00 | N |
| ATOM | 4166 | CA | ILE | D | 199 | 4.053 | −34.984 | 6.950 | 1.00 | 50.00 | C |
| ATOM | 4167 | C | ILE | D | 199 | 5.177 | −34.172 | 7.585 | 1.00 | 50.00 | C |
| ATOM | 4168 | O | ILE | D | 199 | 4.971 | −32.973 | 7.762 | 1.00 | 50.00 | O |
| ATOM | 4169 | CB | ILE | D | 199 | 3.194 | −35.833 | 7.890 | 1.00 | 50.00 | C |
| ATOM | 4170 | CG1 | ILE | D | 199 | 1.785 | −36.036 | 7.329 | 1.00 | 50.00 | C |
| ATOM | 4171 | CG2 | ILE | D | 199 | 3.099 | −35.221 | 9.285 | 1.00 | 50.00 | C |
| ATOM | 4172 | CD1 | ILE | D | 199 | 1.574 | −37.176 | 6.340 | 1.00 | 50.00 | C |
| ATOM | 4173 | N | ASP | D | 200 | 6.329 | −34.737 | 7.911 | 1.00 | 50.00 | N |
| ATOM | 4174 | CA | ASP | D | 200 | 7.572 | −33.992 | 8.119 | 1.00 | 50.00 | C |
| ATOM | 4175 | C | ASP | D | 200 | 7.777 | −32.821 | 7.163 | 1.00 | 50.00 | C |
| ATOM | 4176 | O | ASP | D | 200 | 7.550 | −31.673 | 7.541 | 1.00 | 50.00 | O |
| ATOM | 4177 | CB | ASP | D | 200 | 8.765 | −34.942 | 8.004 | 1.00 | 50.00 | C |
| ATOM | 4178 | CG | ASP | D | 200 | 9.075 | −35.899 | 9.140 | 1.00 | 50.00 | C |
| ATOM | 4179 | OD1 | ASP | D | 200 | 9.881 | −35.475 | 9.996 | 1.00 | 50.00 | O |
| ATOM | 4180 | OD2 | ASP | D | 200 | 8.580 | −37.046 | 9.168 | 1.00 | 50.00 | O |
| ATOM | 4181 | N | LYS | D | 201 | 8.049 | −33.110 | 5.901 | 1.00 | 50.00 | N |
| ATOM | 4182 | CA | LYS | D | 201 | 8.141 | −32.145 | 4.805 | 1.00 | 50.00 | C |
| ATOM | 4183 | C | LYS | D | 201 | 6.963 | −31.235 | 4.483 | 1.00 | 50.00 | C |
| ATOM | 4184 | O | LYS | D | 201 | 7.127 | −30.362 | 3.631 | 1.00 | 50.00 | O |
| ATOM | 4185 | CB | LYS | D | 201 | 8.330 | −32.927 | 3.506 | 1.00 | 50.00 | C |
| ATOM | 4186 | CG | LYS | D | 201 | 9.586 | −33.789 | 3.420 | 1.00 | 50.00 | C |
| ATOM | 4187 | CD | LYS | D | 201 | 9.586 | −34.480 | 2.060 | 1.00 | 50.00 | C |
| ATOM | 4188 | CE | LYS | D | 201 | 10.687 | −35.535 | 2.013 | 1.00 | 50.00 | C |
| ATOM | 4189 | NZ | LYS | D | 201 | 10.798 | −36.026 | 0.633 | 1.00 | 50.00 | N |
| ATOM | 4190 | N | GLN | D | 202 | 5.808 | −31.459 | 5.084 | 1.00 | 50.00 | N |
| ATOM | 4191 | CA | GLN | D | 202 | 4.607 | −30.638 | 4.951 | 1.00 | 50.00 | C |
| ATOM | 4192 | C | GLN | D | 202 | 4.173 | −29.815 | 6.156 | 1.00 | 50.00 | C |
| ATOM | 4193 | O | GLN | D | 202 | 3.575 | −28.754 | 5.986 | 1.00 | 50.00 | O |
| ATOM | 4194 | CB | GLN | D | 202 | 3.482 | −31.620 | 4.640 | 1.00 | 50.00 | C |
| ATOM | 4195 | CG | GLN | D | 202 | 2.533 | −30.986 | 3.631 | 1.00 | 50.00 | C |
| ATOM | 4196 | CD | GLN | D | 202 | 2.904 | −30.818 | 2.167 | 1.00 | 50.00 | C |
| ATOM | 4197 | OE1 | GLN | D | 202 | 4.030 | −31.024 | 1.717 | 1.00 | 50.00 | O |
| ATOM | 4198 | NE2 | GLN | D | 202 | 1.856 | −30.558 | 1.408 | 1.00 | 50.00 | N |
| ATOM | 4199 | N | LEU | D | 203 | 4.531 | −30.254 | 7.349 | 1.00 | 50.00 | N |
| ATOM | 4200 | CA | LEU | D | 203 | 4.512 | −29.472 | 8.582 | 1.00 | 50.00 | C |
| ATOM | 4201 | C | LEU | D | 203 | 5.718 | −28.544 | 8.669 | 1.00 | 50.00 | C |
| ATOM | 4202 | O | LEU | D | 203 | 5.450 | −27.394 | 9.007 | 1.00 | 50.00 | O |
| ATOM | 4203 | CB | LEU | D | 203 | 4.391 | −30.486 | 9.718 | 1.00 | 50.00 | C |
| ATOM | 4204 | CG | LEU | D | 203 | 3.032 | −31.165 | 9.906 | 1.00 | 50.00 | C |
| ATOM | 4205 | CD1 | LEU | D | 203 | 2.191 | −31.577 | 8.697 | 1.00 | 50.00 | C |
| ATOM | 4206 | CD2 | LEU | D | 203 | 3.299 | −32.436 | 10.707 | 1.00 | 50.00 | C |
| ATOM | 4207 | N | LEU | D | 204 | 6.936 | −28.909 | 8.301 | 1.00 | 50.00 | N |
| ATOM | 4208 | CA | LEU | D | 204 | 8.088 | −28.034 | 8.075 | 1.00 | 50.00 | C |
| ATOM | 4209 | C | LEU | D | 204 | 8.033 | −26.864 | 7.090 | 1.00 | 50.00 | C |
| ATOM | 4210 | O | LEU | D | 204 | 8.594 | −25.829 | 7.447 | 1.00 | 50.00 | O |
| ATOM | 4211 | CB | LEU | D | 204 | 9.273 | −28.946 | 7.751 | 1.00 | 50.00 | C |
| ATOM | 4212 | CG | LEU | D | 204 | 9.995 | −29.684 | 8.880 | 1.00 | 50.00 | C |
| ATOM | 4213 | CD1 | LEU | D | 204 | 9.187 | −30.474 | 9.909 | 1.00 | 50.00 | C |
| ATOM | 4214 | CD2 | LEU | D | 204 | 11.050 | −30.597 | 8.256 | 1.00 | 50.00 | C |
| ATOM | 4215 | N | PRO | D | 205 | 7.472 | −26.873 | 5.888 | 1.00 | 50.00 | N |
| ATOM | 4216 | CA | PRO | D | 205 | 7.054 | −25.694 | 5.130 | 1.00 | 50.00 | C |
| ATOM | 4217 | C | PRO | D | 205 | 6.037 | −24.871 | 5.911 | 1.00 | 50.00 | C |
| ATOM | 4218 | O | PRO | D | 205 | 6.433 | −23.762 | 6.263 | 1.00 | 50.00 | O |
| ATOM | 4219 | CB | PRO | D | 205 | 6.563 | −26.232 | 3.786 | 1.00 | 50.00 | C |
| ATOM | 4220 | CG | PRO | D | 205 | 5.988 | −27.611 | 4.083 | 1.00 | 50.00 | C |
| ATOM | 4221 | CD | PRO | D | 205 | 6.688 | −27.994 | 5.384 | 1.00 | 50.00 | C |
| ATOM | 4222 | N | ILE | D | 206 | 4.890 | −25.384 | 6.328 | 1.00 | 50.00 | N |
| ATOM | 4223 | CA | ILE | D | 206 | 4.010 | −24.757 | 7.316 | 1.00 | 50.00 | C |
| ATOM | 4224 | C | ILE | D | 206 | 4.687 | −24.134 | 8.536 | 1.00 | 50.00 | C |
| ATOM | 4225 | O | ILE | D | 206 | 4.320 | −23.011 | 8.880 | 1.00 | 50.00 | O |
| ATOM | 4226 | CB | ILE | D | 206 | 2.800 | −25.626 | 7.668 | 1.00 | 50.00 | C |
| ATOM | 4227 | CG1 | ILE | D | 206 | 1.973 | −26.211 | 6.520 | 1.00 | 50.00 | C |
| ATOM | 4228 | CG2 | ILE | D | 206 | 1.849 | −24.789 | 8.522 | 1.00 | 50.00 | C |
| ATOM | 4229 | CD1 | ILE | D | 206 | 1.092 | −27.417 | 6.847 | 1.00 | 50.00 | C |
| ATOM | 4230 | N | VAL | D | 207 | 5.641 | −24.780 | 9.187 | 1.00 | 50.00 | N |
| ATOM | 4231 | CA | VAL | D | 207 | 6.485 | −24.265 | 10.267 | 1.00 | 50.00 | C |
| ATOM | 4232 | C | VAL | D | 207 | 7.066 | −22.895 | 9.920 | 1.00 | 50.00 | C |
| ATOM | 4233 | O | VAL | D | 207 | 6.735 | −21.924 | 10.599 | 1.00 | 50.00 | O |
| ATOM | 4234 | CB | VAL | D | 207 | 7.498 | −25.350 | 10.650 | 1.00 | 50.00 | C |
| ATOM | 4235 | CG1 | VAL | D | 207 | 8.710 | −24.847 | 11.429 | 1.00 | 50.00 | C |
| ATOM | 4236 | CG2 | VAL | D | 207 | 7.103 | −26.579 | 11.466 | 1.00 | 50.00 | C |
| ATOM | 4237 | N | ASN | D | 208 | 7.872 | −22.793 | 8.875 | 1.00 | 50.00 | N |
| ATOM | 4238 | CA | ASN | D | 208 | 8.431 | −21.535 | 8.374 | 1.00 | 50.00 | C |
| ATOM | 4239 | C | ASN | D | 208 | 7.545 | −20.635 | 7.518 | 1.00 | 50.00 | C |
| ATOM | 4240 | O | ASN | D | 208 | 7.791 | −19.430 | 7.490 | 1.00 | 50.00 | O |
| ATOM | 4241 | CB | ASN | D | 208 | 9.747 | −21.809 | 7.644 | 1.00 | 50.00 | C |
| ATOM | 4242 | CG | ASN | D | 208 | 10.951 | −22.253 | 8.464 | 1.00 | 50.00 | C |
| ATOM | 4243 | OD1 | ASN | D | 208 | 11.593 | −23.259 | 8.165 | 1.00 | 50.00 | O |
| ATOM | 4244 | ND2 | ASN | D | 208 | 11.319 | −21.510 | 9.492 | 1.00 | 50.00 | N |
| ATOM | 4245 | N | LYS | D | 209 | 6.522 | −21.126 | 6.839 | 1.00 | 50.00 | N |
| ATOM | 4246 | CA | LYS | D | 209 | 5.396 | −20.342 | 6.328 | 1.00 | 50.00 | C |
| ATOM | 4247 | C | LYS | D | 209 | 4.649 | −19.535 | 7.383 | 1.00 | 50.00 | C |
| ATOM | 4248 | O | LYS | D | 209 | 4.342 | −18.367 | 7.149 | 1.00 | 50.00 | O |
| ATOM | 4249 | CB | LYS | D | 209 | 4.400 | −21.238 | 5.590 | 1.00 | 50.00 | C |
| ATOM | 4250 | CG | LYS | D | 209 | 4.893 | −21.839 | 4.276 | 1.00 | 50.00 | C |
| ATOM | 4251 | CD | LYS | D | 209 | 3.917 | −22.841 | 3.661 | 1.00 | 50.00 | C |
| ATOM | 4252 | CE | LYS | D | 209 | 4.506 | −23.415 | 2.375 | 1.00 | 50.00 | C |
| ATOM | 4253 | NZ | LYS | D | 209 | 3.675 | −24.528 | 1.896 | 1.00 | 50.00 | N |
| ATOM | 4254 | N | GLN | D | 210 | 4.419 | −20.112 | 8.551 | 1.00 | 50.00 | N |
| ATOM | 4255 | CA | GLN | D | 210 | 4.082 | −19.383 | 9.773 | 1.00 | 50.00 | C |
| ATOM | 4256 | C | GLN | D | 210 | 5.090 | −18.339 | 10.242 | 1.00 | 50.00 | C |
| ATOM | 4257 | O | GLN | D | 210 | 4.642 | −17.270 | 10.651 | 1.00 | 50.00 | O |
| ATOM | 4258 | CB | GLN | D | 210 | 3.755 | −20.406 | 10.862 | 1.00 | 50.00 | C |
| ATOM | 4259 | CG | GLN | D | 210 | 2.462 | −20.110 | 11.618 | 1.00 | 50.00 | C |
| ATOM | 4260 | CD | GLN | D | 210 | 2.576 | −18.900 | 12.533 | 1.00 | 50.00 | C |
| ATOM | 4261 | OE1 | GLN | D | 210 | 3.510 | −18.753 | 13.319 | 1.00 | 50.00 | O |
| ATOM | 4262 | NE2 | GLN | D | 210 | 1.628 | −17.989 | 12.427 | 1.00 | 50.00 | N |
| ATOM | 4263 | N | SER | D | 211 | 6.391 | −18.580 | 10.170 | 1.00 | 50.00 | N |
| ATOM | 4264 | CA | SER | D | 211 | 7.428 | −17.582 | 10.435 | 1.00 | 50.00 | C |
| ATOM | 4265 | C | SER | D | 211 | 7.406 | −16.364 | 9.519 | 1.00 | 50.00 | C |
| ATOM | 4266 | O | SER | D | 211 | 7.341 | −15.247 | 10.029 | 1.00 | 50.00 | O |
| ATOM | 4267 | CB | SER | D | 211 | 8.822 | −18.208 | 10.457 | 1.00 | 50.00 | C |
| ATOM | 4268 | OG | SER | D | 211 | 8.921 | −19.236 | 11.433 | 1.00 | 50.00 | O |
| ATOM | 4269 | N | CYS | D | 212 | 7.399 | −16.522 | 8.205 | 1.00 | 50.00 | N |
| ATOM | 4270 | CA | CYS | D | 212 | 7.207 | −15.444 | 7.234 | 1.00 | 50.00 | C |
| ATOM | 4271 | C | CYS | D | 212 | 6.039 | −14.495 | 7.481 | 1.00 | 50.00 | C |
| ATOM | 4272 | O | CYS | D | 212 | 6.264 | −13.287 | 7.531 | 1.00 | 50.00 | O |
| ATOM | 4273 | CB | CYS | D | 212 | 7.167 | −16.089 | 5.849 | 1.00 | 50.00 | C |
| ATOM | 4274 | SG | CYS | D | 212 | 7.896 | −15.039 | 4.583 | 1.00 | 50.00 | S |
| ATOM | 4275 | N | SER | D | 213 | 4.850 | −15.013 | 7.747 | 1.00 | 50.00 | N |
| ATOM | 4276 | CA | SER | D | 213 | 3.738 | −14.279 | 8.353 | 1.00 | 50.00 | C |
| ATOM | 4277 | C | SER | D | 213 | 4.031 | −13.630 | 9.702 | 1.00 | 50.00 | C |
| ATOM | 4278 | O | SER | D | 213 | 4.022 | −12.400 | 9.705 | 1.00 | 50.00 | O |
| ATOM | 4279 | CB | SER | D | 213 | 2.489 | −15.157 | 8.404 | 1.00 | 50.00 | C |
| ATOM | 4280 | OG | SER | D | 213 | 1.968 | −15.365 | 7.099 | 1.00 | 50.00 | O |
| ATOM | 4281 | N | ILE | D | 214 | 4.336 | −14.336 | 10.781 | 1.00 | 50.00 | N |
| ATOM | 4282 | CA | ILE | D | 214 | 4.792 | −13.789 | 12.064 | 1.00 | 50.00 | C |
| ATOM | 4283 | C | ILE | D | 214 | 5.668 | −12.539 | 12.036 | 1.00 | 50.00 | C |
| ATOM | 4284 | O | ILE | D | 214 | 5.454 | −11.569 | 12.763 | 1.00 | 50.00 | O |
| ATOM | 4285 | CB | ILE | D | 214 | 5.304 | −14.877 | 13.019 | 1.00 | 50.00 | C |
| ATOM | 4286 | CG1 | ILE | D | 214 | 4.563 | −14.876 | 14.357 | 1.00 | 50.00 | C |
| ATOM | 4287 | CG2 | ILE | D | 214 | 6.810 | −14.937 | 13.288 | 1.00 | 50.00 | C |
| ATOM | 4288 | CD1 | ILE | D | 214 | 4.763 | −16.092 | 15.259 | 1.00 | 50.00 | C |
| ATOM | 4289 | N | SER | D | 215 | 6.667 | −12.615 | 11.174 | 1.00 | 50.00 | N |
| ATOM | 4290 | CA | SER | D | 215 | 7.692 | −11.603 | 10.925 | 1.00 | 50.00 | C |
| ATOM | 4291 | C | SER | D | 215 | 7.191 | −10.451 | 10.065 | 1.00 | 50.00 | C |
| ATOM | 4292 | O | SER | D | 215 | 7.427 | −9.306 | 10.447 | 1.00 | 50.00 | O |
| ATOM | 4293 | CB | SER | D | 215 | 8.866 | −12.304 | 10.242 | 1.00 | 50.00 | C |
| ATOM | 4294 | OG | SER | D | 215 | 9.231 | −13.481 | 10.948 | 1.00 | 50.00 | O |
| ATOM | 4295 | N | ASN | D | 216 | 6.480 | −10.698 | 8.977 | 1.00 | 50.00 | N |
| ATOM | 4296 | CA | ASN | D | 216 | 5.762 | −9.666 | 8.229 | 1.00 | 50.00 | C |
| ATOM | 4297 | C | ASN | D | 216 | 4.843 | −8.809 | 9.084 | 1.00 | 50.00 | C |
| ATOM | 4298 | O | ASN | D | 216 | 5.205 | −7.652 | 9.294 | 1.00 | 50.00 | O |
| ATOM | 4299 | CB | ASN | D | 216 | 5.082 | −10.164 | 6.953 | 1.00 | 50.00 | C |
| ATOM | 4300 | CG | ASN | D | 216 | 5.989 | −10.690 | 5.853 | 1.00 | 50.00 | C |
| ATOM | 4301 | OD1 | ASN | D | 216 | 5.720 | −11.645 | 5.126 | 1.00 | 50.00 | O |
| ATOM | 4302 | ND2 | ASN | D | 216 | 7.122 | −10.025 | 5.747 | 1.00 | 50.00 | N |
| ATOM | 4303 | N | ILE | D | 217 | 3.798 | −9.377 | 9.660 | 1.00 | 50.00 | N |
| ATOM | 4304 | CA | ILE | D | 217 | 3.065 | −8.837 | 10.806 | 1.00 | 50.00 | C |
| ATOM | 4305 | C | ILE | D | 217 | 3.813 | −7.924 | 11.774 | 1.00 | 50.00 | C |
| ATOM | 4306 | O | ILE | D | 217 | 3.177 | −7.032 | 12.333 | 1.00 | 50.00 | O |
| ATOM | 4307 | CB | ILE | D | 217 | 2.399 | −9.984 | 11.570 | 1.00 | 50.00 | C |
| ATOM | 4308 | CG1 | ILE | D | 217 | 1.531 | −10.905 | 10.713 | 1.00 | 50.00 | C |
| ATOM | 4309 | CG2 | ILE | D | 217 | 1.535 | −9.496 | 12.733 | 1.00 | 50.00 | C |
| ATOM | 4310 | CD1 | ILE | D | 217 | 1.144 | −12.224 | 11.375 | 1.00 | 50.00 | C |
| ATOM | 4311 | N | GLU | D | 218 | 5.094 | −8.132 | 12.026 | 1.00 | 50.00 | N |
| ATOM | 4312 | CA | GLU | D | 218 | 5.960 | −7.181 | 12.722 | 1.00 | 50.00 | C |
| ATOM | 4313 | C | GLU | D | 218 | 6.537 | −5.947 | 12.055 | 1.00 | 50.00 | C |
| ATOM | 4314 | O | GLU | D | 218 | 6.366 | −4.805 | 12.481 | 1.00 | 50.00 | O |
| ATOM | 4315 | CB | GLU | D | 218 | 7.083 | −7.944 | 13.430 | 1.00 | 50.00 | C |
| ATOM | 4316 | CG | GLU | D | 218 | 6.642 | −8.708 | 14.674 | 1.00 | 50.00 | C |
| ATOM | 4317 | CD | GLU | D | 218 | 6.000 | −7.625 | 15.522 | 1.00 | 50.00 | C |
| ATOM | 4318 | OE1 | GLU | D | 218 | 4.778 | −7.517 | 15.288 | 1.00 | 50.00 | O |
| ATOM | 4319 | OE2 | GLU | D | 218 | 6.629 | −6.823 | 16.246 | 1.00 | 50.00 | O |
| ATOM | 4320 | N | THR | D | 219 | 7.288 | −6.281 | 11.024 | 1.00 | 50.00 | N |
| ATOM | 4321 | CA | THR | D | 219 | 7.932 | −5.343 | 10.110 | 1.00 | 50.00 | C |
| ATOM | 4322 | C | THR | D | 219 | 6.921 | −4.319 | 9.610 | 1.00 | 50.00 | C |
| ATOM | 4323 | O | THR | D | 219 | 7.080 | −3.139 | 9.917 | 1.00 | 50.00 | O |
| ATOM | 4324 | CB | THR | D | 219 | 8.631 | −6.223 | 9.074 | 1.00 | 50.00 | C |
| ATOM | 4325 | OG1 | THR | D | 219 | 7.732 | −6.890 | 8.201 | 1.00 | 50.00 | O |
| ATOM | 4326 | CG2 | THR | D | 219 | 9.606 | −7.245 | 9.660 | 1.00 | 50.00 | C |
| ATOM | 4327 | N | VAL | D | 220 | 5.826 | −4.805 | 9.048 | 1.00 | 50.00 | N |
| ATOM | 4328 | CA | VAL | D | 220 | 4.586 | −4.075 | 8.793 | 1.00 | 50.00 | C |
| ATOM | 4329 | C | VAL | D | 220 | 3.989 | −3.072 | 9.772 | 1.00 | 50.00 | C |
| ATOM | 4330 | O | VAL | D | 220 | 3.121 | −2.296 | 9.377 | 1.00 | 50.00 | O |
| ATOM | 4331 | CB | VAL | D | 220 | 3.461 | −5.004 | 8.339 | 1.00 | 50.00 | C |
| ATOM | 4332 | CG1 | VAL | D | 220 | 3.748 | −6.095 | 7.315 | 1.00 | 50.00 | C |
| ATOM | 4333 | CG2 | VAL | D | 220 | 2.374 | −5.608 | 9.225 | 1.00 | 50.00 | C |
| ATOM | 4334 | N | ILE | D | 221 | 4.302 | −3.130 | 11.053 | 1.00 | 50.00 | N |
| ATOM | 4335 | CA | ILE | D | 221 | 3.848 | −2.154 | 12.045 | 1.00 | 50.00 | C |
| ATOM | 4336 | C | ILE | D | 221 | 4.958 | −1.135 | 12.267 | 1.00 | 50.00 | C |
| ATOM | 4337 | O | ILE | D | 221 | 4.779 | 0.079 | 12.188 | 1.00 | 50.00 | O |
| ATOM | 4338 | CB | ILE | D | 221 | 3.344 | −2.862 | 13.307 | 1.00 | 50.00 | C |
| ATOM | 4339 | CG1 | ILE | D | 221 | 1.932 | −3.415 | 13.120 | 1.00 | 50.00 | C |
| ATOM | 4340 | CG2 | ILE | D | 221 | 3.243 | −2.041 | 14.589 | 1.00 | 50.00 | C |
| ATOM | 4341 | CD1 | ILE | D | 221 | 1.847 | −4.377 | 11.941 | 1.00 | 50.00 | C |
| ATOM | 4342 | N | GLU | D | 222 | 6.123 | −1.677 | 12.569 | 1.00 | 50.00 | N |
| ATOM | 4343 | CA | GLU | D | 222 | 7.397 | −0.989 | 12.763 | 1.00 | 50.00 | C |
| ATOM | 4344 | C | GLU | D | 222 | 7.962 | −0.105 | 11.655 | 1.00 | 50.00 | C |
| ATOM | 4345 | O | GLU | D | 222 | 8.872 | 0.705 | 11.824 | 1.00 | 50.00 | O |
| ATOM | 4346 | CB | GLU | D | 222 | 8.269 | −2.191 | 13.133 | 1.00 | 50.00 | C |
| ATOM | 4347 | CG | GLU | D | 222 | 9.331 | −2.024 | 14.215 | 1.00 | 50.00 | C |
| ATOM | 4348 | CD | GLU | D | 222 | 10.091 | −0.713 | 14.155 | 1.00 | 50.00 | C |
| ATOM | 4349 | OE1 | GLU | D | 222 | 9.665 | 0.160 | 14.940 | 1.00 | 50.00 | O |
| ATOM | 4350 | OE2 | GLU | D | 222 | 11.000 | −0.522 | 13.317 | 1.00 | 50.00 | O |
| ATOM | 4351 | N | PHE | D | 223 | 7.369 | −0.220 | 10.483 | 1.00 | 50.00 | N |
| ATOM | 4352 | CA | PHE | D | 223 | 7.521 | 0.632 | 9.306 | 1.00 | 50.00 | C |
| ATOM | 4353 | C | PHE | D | 223 | 6.310 | 1.558 | 9.143 | 1.00 | 50.00 | C |
| ATOM | 4354 | O | PHE | D | 223 | 6.500 | 2.670 | 8.650 | 1.00 | 50.00 | O |
| ATOM | 4355 | CB | PHE | D | 223 | 7.689 | −0.464 | 8.250 | 1.00 | 50.00 | C |
| ATOM | 4356 | CG | PHE | D | 223 | 8.713 | −1.284 | 7.492 | 1.00 | 50.00 | C |
| ATOM | 4357 | CD1 | PHE | D | 223 | 9.697 | −0.723 | 6.759 | 1.00 | 50.00 | C |
| ATOM | 4358 | CD2 | PHE | D | 223 | 8.445 | −2.591 | 7.277 | 1.00 | 50.00 | C |
| ATOM | 4359 | CE1 | PHE | D | 223 | 10.528 | −1.489 | 6.023 | 1.00 | 50.00 | C |
| ATOM | 4360 | CE2 | PHE | D | 223 | 9.303 | −3.387 | 6.610 | 1.00 | 50.00 | C |
| ATOM | 4361 | CZ | PHE | D | 223 | 10.382 | −2.829 | 6.028 | 1.00 | 50.00 | C |
| ATOM | 4362 | N | GLN | D | 224 | 5.076 | 1.210 | 9.480 | 1.00 | 50.00 | N |
| ATOM | 4363 | CA | GLN | D | 224 | 3.853 | 2.018 | 9.438 | 1.00 | 50.00 | C |
| ATOM | 4364 | C | GLN | D | 224 | 3.716 | 3.146 | 10.450 | 1.00 | 50.00 | C |
| ATOM | 4365 | O | GLN | D | 224 | 3.416 | 4.289 | 10.106 | 1.00 | 50.00 | O |
| ATOM | 4366 | CB | GLN | D | 224 | 2.609 | 1.167 | 9.703 | 1.00 | 50.00 | C |
| ATOM | 4367 | CG | GLN | D | 224 | 1.939 | 0.320 | 8.632 | 1.00 | 50.00 | C |
| ATOM | 4368 | CD | GLN | D | 224 | 1.540 | 1.190 | 7.457 | 1.00 | 50.00 | C |
| ATOM | 4369 | OE1 | GLN | D | 224 | 1.453 | 2.417 | 7.428 | 1.00 | 50.00 | O |
| ATOM | 4370 | NE2 | GLN | D | 224 | 1.309 | 0.387 | 6.441 | 1.00 | 50.00 | N |
| ATOM | 4371 | N | GLN | D | 225 | 3.887 | 2.770 | 11.706 | 1.00 | 50.00 | N |
| ATOM | 4372 | CA | GLN | D | 225 | 4.084 | 3.717 | 12.798 | 1.00 | 50.00 | C |
| ATOM | 4373 | C | GLN | D | 225 | 5.315 | 4.602 | 12.623 | 1.00 | 50.00 | C |
| ATOM | 4374 | O | GLN | D | 225 | 5.418 | 5.602 | 13.331 | 1.00 | 50.00 | O |
| ATOM | 4375 | CB | GLN | D | 225 | 4.199 | 2.967 | 14.122 | 1.00 | 50.00 | C |
| ATOM | 4376 | CG | GLN | D | 225 | 3.233 | 2.172 | 14.992 | 1.00 | 50.00 | C |
| ATOM | 4377 | CD | GLN | D | 225 | 1.983 | 2.921 | 15.423 | 1.00 | 50.00 | C |
| ATOM | 4378 | OE1 | GLN | D | 225 | 2.016 | 4.060 | 15.883 | 1.00 | 50.00 | O |
| ATOM | 4379 | NE2 | GLN | D | 225 | 0.864 | 2.226 | 15.391 | 1.00 | 50.00 | N |
| ATOM | 4380 | N | LYS | D | 226 | 6.276 | 4.296 | 11.764 | 1.00 | 50.00 | N |
| ATOM | 4381 | CA | LYS | D | 226 | 7.360 | 5.203 | 11.395 | 1.00 | 50.00 | C |
| ATOM | 4382 | C | LYS | D | 226 | 6.796 | 6.354 | 10.579 | 1.00 | 50.00 | C |
| ATOM | 4383 | O | LYS | D | 226 | 7.031 | 7.506 | 10.938 | 1.00 | 50.00 | O |
| ATOM | 4384 | CB | LYS | D | 226 | 8.534 | 4.559 | 10.655 | 1.00 | 50.00 | C |
| ATOM | 4385 | CG | LYS | D | 226 | 9.680 | 5.516 | 10.321 | 1.00 | 50.00 | C |
| ATOM | 4386 | CD | LYS | D | 226 | 10.416 | 5.915 | 11.598 | 1.00 | 50.00 | C |
| ATOM | 4387 | CE | LYS | D | 226 | 11.456 | 7.032 | 11.580 | 1.00 | 50.00 | C |
| ATOM | 4388 | NZ | LYS | D | 226 | 12.158 | 7.108 | 12.869 | 1.00 | 50.00 | N |
| ATOM | 4389 | N | ASN | D | 227 | 6.077 | 6.029 | 9.520 | 1.00 | 50.00 | N |
| ATOM | 4390 | CA | ASN | D | 227 | 5.464 | 7.024 | 8.649 | 1.00 | 50.00 | C |
| ATOM | 4391 | C | ASN | D | 227 | 4.564 | 8.018 | 9.377 | 1.00 | 50.00 | C |
| ATOM | 4392 | O | ASN | D | 227 | 4.953 | 9.185 | 9.376 | 1.00 | 50.00 | O |
| ATOM | 4393 | CB | ASN | D | 227 | 4.907 | 6.178 | 7.505 | 1.00 | 50.00 | C |
| ATOM | 4394 | CG | ASN | D | 227 | 4.651 | 7.142 | 6.359 | 1.00 | 50.00 | C |
| ATOM | 4395 | OD1 | ASN | D | 227 | 5.433 | 7.512 | 5.485 | 1.00 | 50.00 | O |
| ATOM | 4396 | ND2 | ASN | D | 227 | 3.553 | 7.830 | 6.580 | 1.00 | 50.00 | N |
| ATOM | 4397 | N | ASN | D | 228 | 3.471 | 7.669 | 10.038 | 1.00 | 50.00 | N |
| ATOM | 4398 | CA | ASN | D | 228 | 2.785 | 8.570 | 10.968 | 1.00 | 50.00 | C |
| ATOM | 4399 | C | ASN | D | 228 | 3.647 | 9.388 | 11.928 | 1.00 | 50.00 | C |
| ATOM | 4400 | O | ASN | D | 228 | 3.358 | 10.570 | 12.101 | 1.00 | 50.00 | O |
| ATOM | 4401 | CB | ASN | D | 228 | 1.624 | 7.860 | 11.667 | 1.00 | 50.00 | C |
| ATOM | 4402 | CG | ASN | D | 228 | 1.883 | 6.624 | 12.516 | 1.00 | 50.00 | C |
| ATOM | 4403 | OD1 | ASN | D | 228 | 1.191 | 5.610 | 12.445 | 1.00 | 50.00 | O |
| ATOM | 4404 | ND2 | ASN | D | 228 | 2.888 | 6.713 | 13.366 | 1.00 | 50.00 | N |
| ATOM | 4405 | N | ARG | D | 229 | 4.714 | 8.835 | 12.485 | 1.00 | 50.00 | N |
| ATOM | 4406 | CA | ARG | D | 229 | 5.673 | 9.496 | 13.372 | 1.00 | 50.00 | C |
| ATOM | 4407 | C | ARG | D | 229 | 6.436 | 10.598 | 12.649 | 1.00 | 50.00 | C |
| ATOM | 4408 | O | ARG | D | 229 | 6.243 | 11.772 | 12.957 | 1.00 | 50.00 | O |
| ATOM | 4409 | CB | ARG | D | 229 | 6.613 | 8.454 | 13.981 | 1.00 | 50.00 | C |
| ATOM | 4410 | CG | ARG | D | 229 | 7.371 | 8.862 | 15.243 | 1.00 | 50.00 | C |
| ATOM | 4411 | CD | ARG | D | 229 | 8.652 | 8.152 | 15.676 | 1.00 | 50.00 | C |
| ATOM | 4412 | NE | ARG | D | 229 | 8.473 | 6.711 | 15.524 | 1.00 | 50.00 | N |
| ATOM | 4413 | CZ | ARG | D | 229 | 9.430 | 5.897 | 15.119 | 1.00 | 50.00 | C |
| ATOM | 4414 | NH1 | ARG | D | 229 | 10.714 | 6.021 | 15.396 | 1.00 | 50.00 | N |
| ATOM | 4415 | NH2 | ARG | D | 229 | 9.039 | 4.867 | 14.398 | 1.00 | 50.00 | N |
| ATOM | 4416 | N | LEU | D | 230 | 7.201 | 10.240 | 11.631 | 1.00 | 50.00 | N |
| ATOM | 4417 | CA | LEU | D | 230 | 7.710 | 11.114 | 10.576 | 1.00 | 50.00 | C |
| ATOM | 4418 | C | LEU | D | 230 | 6.852 | 12.276 | 10.094 | 1.00 | 50.00 | C |
| ATOM | 4419 | O | LEU | D | 230 | 7.306 | 13.420 | 10.087 | 1.00 | 50.00 | O |
| ATOM | 4420 | CB | LEU | D | 230 | 8.016 | 10.190 | 9.397 | 1.00 | 50.00 | C |
| ATOM | 4421 | CG | LEU | D | 230 | 9.369 | 10.424 | 8.732 | 1.00 | 50.00 | C |
| ATOM | 4422 | CD1 | LEU | D | 230 | 10.506 | 9.982 | 9.651 | 1.00 | 50.00 | C |
| ATOM | 4423 | CD2 | LEU | D | 230 | 9.359 | 9.552 | 7.479 | 1.00 | 50.00 | C |
| ATOM | 4424 | N | LEU | D | 231 | 5.619 | 11.975 | 9.724 | 1.00 | 50.00 | N |
| ATOM | 4425 | CA | LEU | D | 231 | 4.623 | 12.967 | 9.329 | 1.00 | 50.00 | C |
| ATOM | 4426 | C | LEU | D | 231 | 4.252 | 13.847 | 10.514 | 1.00 | 50.00 | C |
| ATOM | 4427 | O | LEU | D | 231 | 4.589 | 15.021 | 10.386 | 1.00 | 50.00 | O |
| ATOM | 4428 | CB | LEU | D | 231 | 3.451 | 12.269 | 8.644 | 1.00 | 50.00 | C |
| ATOM | 4429 | CG | LEU | D | 231 | 3.690 | 11.857 | 7.191 | 1.00 | 50.00 | C |
| ATOM | 4430 | CD1 | LEU | D | 231 | 4.832 | 10.930 | 6.782 | 1.00 | 50.00 | C |
| ATOM | 4431 | CD2 | LEU | D | 231 | 2.378 | 11.205 | 6.769 | 1.00 | 50.00 | C |
| ATOM | 4432 | N | GLU | D | 232 | 3.780 | 13.376 | 11.658 | 1.00 | 50.00 | N |
| ATOM | 4433 | CA | GLU | D | 232 | 3.721 | 14.139 | 12.908 | 1.00 | 50.00 | C |
| ATOM | 4434 | C | GLU | D | 232 | 4.823 | 15.154 | 13.183 | 1.00 | 50.00 | C |
| ATOM | 4435 | O | GLU | D | 232 | 4.550 | 16.342 | 13.348 | 1.00 | 50.00 | O |
| ATOM | 4436 | CB | GLU | D | 232 | 3.707 | 13.245 | 14.150 | 1.00 | 50.00 | C |
| ATOM | 4437 | CG | GLU | D | 232 | 2.932 | 13.778 | 15.353 | 1.00 | 50.00 | C |
| ATOM | 4438 | CD | GLU | D | 232 | 3.675 | 14.587 | 16.406 | 1.00 | 50.00 | C |
| ATOM | 4439 | OE1 | GLU | D | 232 | 4.576 | 15.390 | 16.081 | 1.00 | 50.00 | O |
| ATOM | 4440 | OE2 | GLU | D | 232 | 3.380 | 14.390 | 17.604 | 1.00 | 50.00 | O |
| ATOM | 4441 | N | ILE | D | 233 | 6.043 | 14.643 | 13.242 | 1.00 | 50.00 | N |
| ATOM | 4442 | CA | ILE | D | 233 | 7.266 | 15.359 | 13.599 | 1.00 | 50.00 | C |
| ATOM | 4443 | C | ILE | D | 233 | 7.404 | 16.452 | 12.548 | 1.00 | 50.00 | C |
| ATOM | 4444 | O | ILE | D | 233 | 7.056 | 17.608 | 12.781 | 1.00 | 50.00 | O |
| ATOM | 4445 | CB | ILE | D | 233 | 8.423 | 14.361 | 13.720 | 1.00 | 50.00 | C |
| ATOM | 4446 | CG1 | ILE | D | 233 | 8.221 | 13.337 | 14.838 | 1.00 | 50.00 | C |
| ATOM | 4447 | CG2 | ILE | D | 233 | 9.756 | 15.093 | 13.869 | 1.00 | 50.00 | C |
| ATOM | 4448 | CD1 | ILE | D | 233 | 9.321 | 12.295 | 15.031 | 1.00 | 50.00 | C |
| ATOM | 4449 | N | THR | D | 234 | 7.768 | 16.008 | 11.359 | 1.00 | 50.00 | N |
| ATOM | 4450 | CA | THR | D | 234 | 8.172 | 16.888 | 10.269 | 1.00 | 50.00 | C |
| ATOM | 4451 | C | THR | D | 234 | 7.036 | 17.080 | 9.267 | 1.00 | 50.00 | C |
| ATOM | 4452 | O | THR | D | 234 | 7.232 | 17.012 | 8.055 | 1.00 | 50.00 | O |
| ATOM | 4453 | CB | THR | D | 234 | 9.491 | 16.207 | 9.901 | 1.00 | 50.00 | C |
| ATOM | 4454 | OG1 | THR | D | 234 | 10.477 | 16.179 | 10.923 | 1.00 | 50.00 | O |
| ATOM | 4455 | CG2 | THR | D | 234 | 10.198 | 17.079 | 8.876 | 1.00 | 50.00 | C |
| ATOM | 4456 | N | ARG | D | 235 | 5.841 | 17.419 | 9.726 | 1.00 | 50.00 | N |
| ATOM | 4457 | CA | ARG | D | 235 | 4.683 | 17.751 | 8.894 | 1.00 | 50.00 | C |
| ATOM | 4458 | C | ARG | D | 235 | 4.852 | 19.045 | 8.102 | 1.00 | 50.00 | C |
| ATOM | 4459 | O | ARG | D | 235 | 4.635 | 18.958 | 6.895 | 1.00 | 50.00 | O |
| ATOM | 4460 | CB | ARG | D | 235 | 3.355 | 17.599 | 9.637 | 1.00 | 50.00 | C |
| ATOM | 4461 | CG | ARG | D | 235 | 3.245 | 18.344 | 10.964 | 1.00 | 50.00 | C |
| ATOM | 4462 | CD | ARG | D | 235 | 1.882 | 18.130 | 11.619 | 1.00 | 50.00 | C |
| ATOM | 4463 | NE | ARG | D | 235 | 1.797 | 18.956 | 12.821 | 1.00 | 50.00 | N |
| ATOM | 4464 | CZ | ARG | D | 235 | 1.106 | 20.079 | 12.898 | 1.00 | 50.00 | C |
| ATOM | 4465 | NH1 | ARG | D | 235 | 0.110 | 20.414 | 12.103 | 1.00 | 50.00 | N |
| ATOM | 4466 | NH2 | ARG | D | 235 | 1.460 | 20.981 | 13.789 | 1.00 | 50.00 | N |
| ATOM | 4467 | N | GLU | D | 236 | 5.290 | 20.161 | 8.667 | 1.00 | 50.00 | N |
| ATOM | 4468 | CA | GLU | D | 236 | 5.771 | 21.366 | 7.982 | 1.00 | 50.00 | C |
| ATOM | 4469 | C | GLU | D | 236 | 6.461 | 21.196 | 6.635 | 1.00 | 50.00 | C |
| ATOM | 4470 | O | GLU | D | 236 | 5.920 | 21.528 | 5.581 | 1.00 | 50.00 | O |
| ATOM | 4471 | CB | GLU | D | 236 | 6.686 | 22.343 | 8.729 | 1.00 | 50.00 | C |
| ATOM | 4472 | CG | GLU | D | 236 | 7.903 | 21.885 | 9.530 | 1.00 | 50.00 | C |
| ATOM | 4473 | CD | GLU | D | 236 | 7.361 | 21.141 | 10.734 | 1.00 | 50.00 | C |
| ATOM | 4474 | OE1 | GLU | D | 236 | 7.324 | 19.893 | 10.768 | 1.00 | 50.00 | O |
| ATOM | 4475 | OE2 | GLU | D | 236 | 6.883 | 21.870 | 11.628 | 1.00 | 50.00 | O |
| ATOM | 4476 | N | PHE | D | 237 | 7.685 | 20.699 | 6.708 | 1.00 | 50.00 | N |
| ATOM | 4477 | CA | PHE | D | 237 | 8.583 | 20.469 | 5.579 | 1.00 | 50.00 | C |
| ATOM | 4478 | C | PHE | D | 237 | 7.937 | 19.625 | 4.485 | 1.00 | 50.00 | C |
| ATOM | 4479 | O | PHE | D | 237 | 8.118 | 19.868 | 3.292 | 1.00 | 50.00 | O |
| ATOM | 4480 | CB | PHE | D | 237 | 9.897 | 19.977 | 6.195 | 1.00 | 50.00 | C |
| ATOM | 4481 | CG | PHE | D | 237 | 10.433 | 18.576 | 5.977 | 1.00 | 50.00 | C |
| ATOM | 4482 | CD1 | PHE | D | 237 | 9.638 | 17.509 | 5.763 | 1.00 | 50.00 | C |
| ATOM | 4483 | CD2 | PHE | D | 237 | 11.736 | 18.320 | 6.202 | 1.00 | 50.00 | C |
| ATOM | 4484 | CE1 | PHE | D | 237 | 10.090 | 16.249 | 5.634 | 1.00 | 50.00 | C |
| ATOM | 4485 | CE2 | PHE | D | 237 | 12.205 | 17.056 | 6.240 | 1.00 | 50.00 | C |
| ATOM | 4486 | CZ | PHE | D | 237 | 11.378 | 16.029 | 5.956 | 1.00 | 50.00 | C |
| ATOM | 4487 | N | SER | D | 238 | 7.201 | 18.625 | 4.943 | 1.00 | 50.00 | N |
| ATOM | 4488 | CA | SER | D | 238 | 6.676 | 17.498 | 4.175 | 1.00 | 50.00 | C |
| ATOM | 4489 | C | SER | D | 238 | 5.456 | 17.926 | 3.375 | 1.00 | 50.00 | C |
| ATOM | 4490 | O | SER | D | 238 | 5.009 | 17.162 | 2.523 | 1.00 | 50.00 | O |
| ATOM | 4491 | CB | SER | D | 238 | 6.346 | 16.308 | 5.076 | 1.00 | 50.00 | C |
| ATOM | 4492 | OG | SER | D | 238 | 5.128 | 16.443 | 5.795 | 1.00 | 50.00 | O |
| ATOM | 4493 | N | VAL | D | 239 | 4.918 | 19.098 | 3.667 | 1.00 | 50.00 | N |
| ATOM | 4494 | CA | VAL | D | 239 | 3.812 | 19.723 | 2.954 | 1.00 | 50.00 | C |
| ATOM | 4495 | C | VAL | D | 239 | 4.232 | 21.013 | 2.252 | 1.00 | 50.00 | C |
| ATOM | 4496 | O | VAL | D | 239 | 5.398 | 21.407 | 2.286 | 1.00 | 50.00 | O |
| ATOM | 4497 | CB | VAL | D | 239 | 2.624 | 19.744 | 3.912 | 1.00 | 50.00 | C |
| ATOM | 4498 | CG1 | VAL | D | 239 | 2.240 | 18.411 | 4.551 | 1.00 | 50.00 | C |
| ATOM | 4499 | CG2 | VAL | D | 239 | 2.820 | 20.865 | 4.926 | 1.00 | 50.00 | C |
| ATOM | 4500 | N | ASN | D | 240 | 3.288 | 21.753 | 1.690 | 1.00 | 50.00 | N |
| ATOM | 4501 | CA | ASN | D | 240 | 3.471 | 23.141 | 1.259 | 1.00 | 50.00 | C |
| ATOM | 4502 | C | ASN | D | 240 | 3.648 | 24.127 | 2.414 | 1.00 | 50.00 | C |
| ATOM | 4503 | O | ASN | D | 240 | 4.442 | 25.044 | 2.211 | 1.00 | 50.00 | O |
| ATOM | 4504 | CB | ASN | D | 240 | 2.494 | 23.753 | 0.249 | 1.00 | 50.00 | C |
| ATOM | 4505 | CG | ASN | D | 240 | 1.949 | 23.210 | −1.065 | 1.00 | 50.00 | C |
| ATOM | 4506 | OD1 | ASN | D | 240 | 0.750 | 23.004 | −1.252 | 1.00 | 50.00 | O |
| ATOM | 4507 | ND2 | ASN | D | 240 | 2.787 | 23.032 | −2.067 | 1.00 | 50.00 | N |
| ATOM | 4508 | N | ALA | D | 241 | 2.976 | 24.062 | 3.555 | 1.00 | 50.00 | N |
| ATOM | 4509 | CA | ALA | D | 241 | 3.104 | 25.029 | 4.649 | 1.00 | 50.00 | C |
| ATOM | 4510 | C | ALA | D | 241 | 4.112 | 24.626 | 5.717 | 1.00 | 50.00 | C |
| ATOM | 4511 | O | ALA | D | 241 | 4.205 | 23.457 | 6.088 | 1.00 | 50.00 | O |
| ATOM | 4512 | CB | ALA | D | 241 | 1.847 | 25.270 | 5.486 | 1.00 | 50.00 | C |
| ATOM | 4513 | N | GLY | D | 242 | 4.671 | 25.634 | 6.366 | 1.00 | 50.00 | N |
| ATOM | 4514 | CA | GLY | D | 242 | 5.177 | 25.515 | 7.732 | 1.00 | 50.00 | C |
| ATOM | 4515 | C | GLY | D | 242 | 4.207 | 25.296 | 8.888 | 1.00 | 50.00 | C |
| ATOM | 4516 | O | GLY | D | 242 | 2.991 | 25.456 | 8.780 | 1.00 | 50.00 | O |
| ATOM | 4517 | N | VAL | D | 243 | 4.847 | 24.943 | 9.990 | 1.00 | 50.00 | N |
| ATOM | 4518 | CA | VAL | D | 243 | 4.370 | 24.281 | 11.208 | 1.00 | 50.00 | C |
| ATOM | 4519 | C | VAL | D | 243 | 5.528 | 24.341 | 12.201 | 1.00 | 50.00 | C |
| ATOM | 4520 | O | VAL | D | 243 | 6.642 | 24.774 | 11.905 | 1.00 | 50.00 | O |
| ATOM | 4521 | CB | VAL | D | 243 | 3.958 | 22.823 | 10.989 | 1.00 | 50.00 | C |
| ATOM | 4522 | CG1 | VAL | D | 243 | 3.848 | 21.764 | 12.085 | 1.00 | 50.00 | C |
| ATOM | 4523 | CG2 | VAL | D | 243 | 2.604 | 22.761 | 10.298 | 1.00 | 50.00 | C |
| ATOM | 4524 | N | THR | D | 244 | 5.334 | 23.795 | 13.392 | 1.00 | 50.00 | N |
| ATOM | 4525 | CA | THR | D | 244 | 6.406 | 23.386 | 14.302 | 1.00 | 50.00 | C |
| ATOM | 4526 | C | THR | D | 244 | 6.258 | 21.958 | 14.838 | 1.00 | 50.00 | C |
| ATOM | 4527 | O | THR | D | 244 | 5.231 | 21.341 | 14.562 | 1.00 | 50.00 | O |
| ATOM | 4528 | CB | THR | D | 244 | 6.747 | 24.626 | 15.136 | 1.00 | 50.00 | C |
| ATOM | 4529 | OG1 | THR | D | 244 | 6.296 | 25.884 | 14.651 | 1.00 | 50.00 | O |
| ATOM | 4530 | CG2 | THR | D | 244 | 8.246 | 24.881 | 15.260 | 1.00 | 50.00 | C |
| ATOM | 4531 | N | THR | D | 245 | 7.187 | 21.233 | 15.451 | 1.00 | 50.00 | N |
| ATOM | 4532 | CA | THR | D | 245 | 7.696 | 19.952 | 14.943 | 1.00 | 50.00 | C |
| ATOM | 4533 | C | THR | D | 245 | 8.433 | 18.881 | 15.747 | 1.00 | 50.00 | C |
| ATOM | 4534 | O | THR | D | 245 | 9.019 | 17.985 | 15.142 | 1.00 | 50.00 | O |
| ATOM | 4535 | CB | THR | D | 245 | 8.483 | 20.271 | 13.669 | 1.00 | 50.00 | C |
| ATOM | 4536 | OG1 | THR | D | 245 | 9.083 | 19.206 | 12.948 | 1.00 | 50.00 | O |
| ATOM | 4537 | CG2 | THR | D | 245 | 9.452 | 21.451 | 13.598 | 1.00 | 50.00 | C |
| ATOM | 4538 | N | PRO | D | 246 | 8.200 | 18.473 | 16.983 | 1.00 | 50.00 | N |
| ATOM | 4539 | CA | PRO | D | 246 | 8.882 | 18.993 | 18.168 | 1.00 | 50.00 | C |
| ATOM | 4540 | C | PRO | D | 246 | 10.082 | 18.278 | 18.773 | 1.00 | 50.00 | C |
| ATOM | 4541 | O | PRO | D | 246 | 10.893 | 18.909 | 19.449 | 1.00 | 50.00 | O |
| ATOM | 4542 | CB | PRO | D | 246 | 7.817 | 18.948 | 19.262 | 1.00 | 50.00 | C |
| ATOM | 4543 | CG | PRO | D | 246 | 6.939 | 17.783 | 18.817 | 1.00 | 50.00 | C |
| ATOM | 4544 | CD | PRO | D | 246 | 6.920 | 17.844 | 17.292 | 1.00 | 50.00 | C |
| ATOM | 4545 | N | VAL | D | 247 | 10.106 | 16.957 | 18.817 | 1.00 | 50.00 | N |
| ATOM | 4546 | CA | VAL | D | 247 | 10.585 | 16.071 | 17.755 | 1.00 | 50.00 | C |
| ATOM | 4547 | C | VAL | D | 247 | 11.402 | 16.534 | 16.550 | 1.00 | 50.00 | C |
| ATOM | 4548 | O | VAL | D | 247 | 11.927 | 15.695 | 15.820 | 1.00 | 50.00 | O |
| ATOM | 4549 | CB | VAL | D | 247 | 9.785 | 14.767 | 17.653 | 1.00 | 50.00 | C |
| ATOM | 4550 | CG1 | VAL | D | 247 | 10.487 | 13.468 | 18.048 | 1.00 | 50.00 | C |
| ATOM | 4551 | CG2 | VAL | D | 247 | 8.378 | 14.666 | 18.244 | 1.00 | 50.00 | C |
| ATOM | 4552 | N | SER | D | 248 | 11.597 | 17.823 | 16.326 | 1.00 | 50.00 | N |
| ATOM | 4553 | CA | SER | D | 248 | 12.601 | 18.403 | 15.434 | 1.00 | 50.00 | C |
| ATOM | 4554 | C | SER | D | 248 | 13.549 | 19.305 | 16.215 | 1.00 | 50.00 | C |
| ATOM | 4555 | O | SER | D | 248 | 14.744 | 19.031 | 16.108 | 1.00 | 50.00 | O |
| ATOM | 4556 | CB | SER | D | 248 | 11.951 | 19.111 | 14.248 | 1.00 | 50.00 | C |
| ATOM | 4557 | OG | SER | D | 248 | 11.471 | 18.171 | 13.298 | 1.00 | 50.00 | O |
| ATOM | 4558 | N | THR | D | 249 | 13.135 | 20.301 | 16.986 | 1.00 | 50.00 | N |
| ATOM | 4559 | CA | THR | D | 249 | 14.027 | 21.103 | 17.828 | 1.00 | 50.00 | C |
| ATOM | 4560 | C | THR | D | 249 | 14.924 | 20.314 | 18.776 | 1.00 | 50.00 | C |
| ATOM | 4561 | O | THR | D | 249 | 16.137 | 20.502 | 18.690 | 1.00 | 50.00 | O |
| ATOM | 4562 | CB | THR | D | 249 | 13.318 | 22.266 | 18.528 | 1.00 | 50.00 | C |
| ATOM | 4563 | OG1 | THR | D | 249 | 12.789 | 23.172 | 17.570 | 1.00 | 50.00 | O |
| ATOM | 4564 | CG2 | THR | D | 249 | 14.195 | 23.106 | 19.455 | 1.00 | 50.00 | C |
| ATOM | 4565 | N | TYR | D | 250 | 14.416 | 19.464 | 19.655 | 1.00 | 50.00 | N |
| ATOM | 4566 | CA | TYR | D | 250 | 15.225 | 18.521 | 20.433 | 1.00 | 50.00 | C |
| ATOM | 4567 | C | TYR | D | 250 | 16.085 | 17.573 | 19.602 | 1.00 | 50.00 | C |
| ATOM | 4568 | O | TYR | D | 250 | 17.193 | 17.279 | 20.049 | 1.00 | 50.00 | O |
| ATOM | 4569 | CB | TYR | D | 250 | 14.334 | 17.840 | 21.475 | 1.00 | 50.00 | C |
| ATOM | 4570 | CG | TYR | D | 250 | 14.760 | 16.753 | 22.445 | 1.00 | 50.00 | C |
| ATOM | 4571 | CD1 | TYR | D | 250 | 14.360 | 16.809 | 23.733 | 1.00 | 50.00 | C |
| ATOM | 4572 | CD2 | TYR | D | 250 | 15.350 | 15.617 | 22.019 | 1.00 | 50.00 | C |
| ATOM | 4573 | CE1 | TYR | D | 250 | 14.610 | 15.801 | 24.593 | 1.00 | 50.00 | C |
| ATOM | 4574 | CE2 | TYR | D | 250 | 15.448 | 14.537 | 22.819 | 1.00 | 50.00 | C |
| ATOM | 4575 | CZ | TYR | D | 250 | 15.148 | 14.656 | 24.127 | 1.00 | 50.00 | C |
| ATOM | 4576 | OH | TYR | D | 250 | 15.551 | 13.658 | 24.974 | 1.00 | 50.00 | O |
| ATOM | 4577 | N | MET | D | 251 | 15.632 | 17.064 | 18.466 | 1.00 | 50.00 | N |
| ATOM | 4578 | CA | MET | D | 251 | 16.315 | 16.055 | 17.652 | 1.00 | 50.00 | C |
| ATOM | 4579 | C | MET | D | 251 | 17.584 | 16.601 | 17.011 | 1.00 | 50.00 | C |
| ATOM | 4580 | O | MET | D | 251 | 18.661 | 16.029 | 17.180 | 1.00 | 50.00 | O |
| ATOM | 4581 | CB | MET | D | 251 | 15.465 | 15.465 | 16.524 | 1.00 | 50.00 | C |
| ATOM | 4582 | CG | MET | D | 251 | 14.513 | 14.293 | 16.750 | 1.00 | 50.00 | C |
| ATOM | 4583 | SD | MET | D | 251 | 15.166 | 12.998 | 17.816 | 1.00 | 50.00 | S |
| ATOM | 4584 | CE | MET | D | 251 | 15.985 | 12.018 | 16.548 | 1.00 | 50.00 | C |
| ATOM | 4585 | N | LEU | D | 252 | 17.452 | 17.729 | 16.332 | 1.00 | 50.00 | N |
| ATOM | 4586 | CA | LEU | D | 252 | 18.506 | 18.732 | 16.188 | 1.00 | 50.00 | C |
| ATOM | 4587 | C | LEU | D | 252 | 19.458 | 18.921 | 17.364 | 1.00 | 50.00 | C |
| ATOM | 4588 | O | LEU | D | 252 | 20.655 | 18.685 | 17.207 | 1.00 | 50.00 | O |
| ATOM | 4589 | CB | LEU | D | 252 | 17.821 | 20.030 | 15.758 | 1.00 | 50.00 | C |
| ATOM | 4590 | CG | LEU | D | 252 | 18.715 | 21.262 | 15.635 | 1.00 | 50.00 | C |
| ATOM | 4591 | CD1 | LEU | D | 252 | 19.895 | 20.966 | 14.711 | 1.00 | 50.00 | C |
| ATOM | 4592 | CD2 | LEU | D | 252 | 17.877 | 22.439 | 15.146 | 1.00 | 50.00 | C |
| ATOM | 4593 | N | THR | D | 253 | 18.946 | 19.314 | 18.518 | 1.00 | 50.00 | N |
| ATOM | 4594 | CA | THR | D | 253 | 19.731 | 19.458 | 19.744 | 1.00 | 50.00 | C |
| ATOM | 4595 | C | THR | D | 253 | 20.318 | 18.189 | 20.357 | 1.00 | 50.00 | C |
| ATOM | 4596 | O | THR | D | 253 | 21.016 | 18.285 | 21.367 | 1.00 | 50.00 | O |
| ATOM | 4597 | CB | THR | D | 253 | 18.847 | 20.281 | 20.682 | 1.00 | 50.00 | C |
| ATOM | 4598 | OG1 | THR | D | 253 | 18.507 | 21.519 | 20.076 | 1.00 | 50.00 | O |
| ATOM | 4599 | CG2 | THR | D | 253 | 19.472 | 20.634 | 22.028 | 1.00 | 50.00 | C |
| ATOM | 4600 | N | ASN | D | 254 | 20.111 | 17.013 | 19.788 | 1.00 | 50.00 | N |
| ATOM | 4601 | CA | ASN | D | 254 | 20.584 | 15.721 | 20.287 | 1.00 | 50.00 | C |
| ATOM | 4602 | C | ASN | D | 254 | 20.237 | 15.406 | 21.738 | 1.00 | 50.00 | C |
| ATOM | 4603 | O | ASN | D | 254 | 21.115 | 15.063 | 22.530 | 1.00 | 50.00 | O |
| ATOM | 4604 | CB | ASN | D | 254 | 22.068 | 15.539 | 19.959 | 1.00 | 50.00 | C |
| ATOM | 4605 | CG | ASN | D | 254 | 22.492 | 15.354 | 18.509 | 1.00 | 50.00 | C |
| ATOM | 4606 | OD1 | ASN | D | 254 | 23.406 | 16.019 | 18.026 | 1.00 | 50.00 | O |
| ATOM | 4607 | ND2 | ASN | D | 254 | 21.891 | 14.420 | 17.794 | 1.00 | 50.00 | N |
| ATOM | 4608 | N | SER | D | 255 | 18.980 | 15.547 | 22.127 | 1.00 | 50.00 | N |
| ATOM | 4609 | CA | SER | D | 255 | 18.550 | 15.424 | 23.520 | 1.00 | 50.00 | C |
| ATOM | 4610 | C | SER | D | 255 | 18.926 | 16.468 | 24.565 | 1.00 | 50.00 | C |
| ATOM | 4611 | O | SER | D | 255 | 18.368 | 16.437 | 25.661 | 1.00 | 50.00 | O |
| ATOM | 4612 | CB | SER | D | 255 | 18.637 | 13.983 | 24.025 | 1.00 | 50.00 | C |
| ATOM | 4613 | OG | SER | D | 255 | 18.047 | 13.051 | 23.129 | 1.00 | 50.00 | O |
| ATOM | 4614 | N | GLU | D | 256 | 19.805 | 17.407 | 24.256 | 1.00 | 50.00 | N |
| ATOM | 4615 | CA | GLU | D | 256 | 20.484 | 18.237 | 25.250 | 1.00 | 50.00 | C |
| ATOM | 4616 | C | GLU | D | 256 | 20.001 | 19.681 | 25.190 | 1.00 | 50.00 | C |
| ATOM | 4617 | O | GLU | D | 256 | 20.706 | 20.555 | 24.687 | 1.00 | 50.00 | O |
| ATOM | 4618 | CB | GLU | D | 256 | 21.983 | 18.038 | 25.018 | 1.00 | 50.00 | C |
| ATOM | 4619 | CG | GLU | D | 256 | 22.567 | 16.696 | 25.462 | 1.00 | 50.00 | C |
| ATOM | 4620 | CD | GLU | D | 256 | 22.518 | 16.479 | 26.967 | 1.00 | 50.00 | C |
| ATOM | 4621 | OE1 | GLU | D | 256 | 21.536 | 15.883 | 27.460 | 1.00 | 50.00 | O |
| ATOM | 4622 | OE2 | GLU | D | 256 | 23.450 | 16.923 | 27.672 | 1.00 | 50.00 | O |
| ATOM | 4623 | N | LEU | D | 257 | 18.799 | 19.953 | 25.674 | 1.00 | 50.00 | N |
| ATOM | 4624 | CA | LEU | D | 257 | 18.086 | 21.217 | 25.464 | 1.00 | 50.00 | C |
| ATOM | 4625 | C | LEU | D | 257 | 18.724 | 22.464 | 26.060 | 1.00 | 50.00 | C |
| ATOM | 4626 | O | LEU | D | 257 | 18.834 | 23.505 | 25.415 | 1.00 | 50.00 | O |
| ATOM | 4627 | CB | LEU | D | 257 | 16.623 | 21.126 | 25.903 | 1.00 | 50.00 | C |
| ATOM | 4628 | CG | LEU | D | 257 | 15.668 | 20.506 | 24.885 | 1.00 | 50.00 | C |
| ATOM | 4629 | CD1 | LEU | D | 257 | 16.169 | 19.159 | 24.372 | 1.00 | 50.00 | C |
| ATOM | 4630 | CD2 | LEU | D | 257 | 14.245 | 20.376 | 25.427 | 1.00 | 50.00 | C |
| ATOM | 4631 | N | LEU | D | 258 | 19.177 | 22.284 | 27.288 | 1.00 | 50.00 | N |
| ATOM | 4632 | CA | LEU | D | 258 | 20.176 | 23.084 | 27.994 | 1.00 | 50.00 | C |
| ATOM | 4633 | C | LEU | D | 258 | 21.343 | 23.560 | 27.132 | 1.00 | 50.00 | C |
| ATOM | 4634 | O | LEU | D | 258 | 21.562 | 24.770 | 27.089 | 1.00 | 50.00 | O |
| ATOM | 4635 | CB | LEU | D | 258 | 20.561 | 22.236 | 29.209 | 1.00 | 50.00 | C |
| ATOM | 4636 | CG | LEU | D | 258 | 19.521 | 21.876 | 30.274 | 1.00 | 50.00 | C |
| ATOM | 4637 | CD1 | LEU | D | 258 | 18.395 | 20.919 | 29.879 | 1.00 | 50.00 | C |
| ATOM | 4638 | CD2 | LEU | D | 258 | 20.210 | 21.267 | 31.493 | 1.00 | 50.00 | C |
| ATOM | 4639 | N | SER | D | 259 | 22.000 | 22.703 | 26.366 | 1.00 | 50.00 | N |
| ATOM | 4640 | CA | SER | D | 259 | 22.941 | 23.084 | 25.310 | 1.00 | 50.00 | C |
| ATOM | 4641 | C | SER | D | 259 | 22.408 | 24.003 | 24.217 | 1.00 | 50.00 | C |
| ATOM | 4642 | O | SER | D | 259 | 22.961 | 25.097 | 24.109 | 1.00 | 50.00 | O |
| ATOM | 4643 | CB | SER | D | 259 | 23.645 | 21.871 | 24.701 | 1.00 | 50.00 | C |
| ATOM | 4644 | OG | SER | D | 259 | 24.345 | 21.115 | 25.679 | 1.00 | 50.00 | O |
| ATOM | 4645 | N | LEU | D | 260 | 21.390 | 23.656 | 23.441 | 1.00 | 50.00 | N |
| ATOM | 4646 | CA | LEU | D | 260 | 20.837 | 24.526 | 22.400 | 1.00 | 50.00 | C |
| ATOM | 4647 | C | LEU | D | 260 | 20.379 | 25.901 | 22.866 | 1.00 | 50.00 | C |
| ATOM | 4648 | O | LEU | D | 260 | 20.762 | 26.883 | 22.231 | 1.00 | 50.00 | O |
| ATOM | 4649 | CB | LEU | D | 260 | 19.688 | 23.932 | 21.583 | 1.00 | 50.00 | C |
| ATOM | 4650 | CG | LEU | D | 260 | 19.154 | 24.734 | 20.392 | 1.00 | 50.00 | C |
| ATOM | 4651 | CD1 | LEU | D | 260 | 20.178 | 24.690 | 19.259 | 1.00 | 50.00 | C |
| ATOM | 4652 | CD2 | LEU | D | 260 | 17.726 | 24.397 | 19.965 | 1.00 | 50.00 | C |
| ATOM | 4653 | N | ILE | D | 261 | 19.558 | 25.979 | 23.900 | 1.00 | 50.00 | N |
| ATOM | 4654 | CA | ILE | D | 261 | 19.122 | 27.239 | 24.501 | 1.00 | 50.00 | C |
| ATOM | 4655 | C | ILE | D | 261 | 20.325 | 28.105 | 24.859 | 1.00 | 50.00 | C |
| ATOM | 4656 | O | ILE | D | 261 | 20.444 | 29.163 | 24.244 | 1.00 | 50.00 | O |
| ATOM | 4657 | CB | ILE | D | 261 | 18.108 | 27.002 | 25.623 | 1.00 | 50.00 | C |
| ATOM | 4658 | CG1 | ILE | D | 261 | 16.837 | 26.246 | 25.227 | 1.00 | 50.00 | C |
| ATOM | 4659 | CG2 | ILE | D | 261 | 17.688 | 28.330 | 26.250 | 1.00 | 50.00 | C |
| ATOM | 4660 | CD1 | ILE | D | 261 | 16.072 | 25.685 | 26.424 | 1.00 | 50.00 | C |
| ATOM | 4661 | N | ASN | D | 262 | 21.277 | 27.692 | 25.680 | 1.00 | 50.00 | N |
| ATOM | 4662 | CA | ASN | D | 262 | 22.545 | 28.401 | 25.868 | 1.00 | 50.00 | C |
| ATOM | 4663 | C | ASN | D | 262 | 23.287 | 28.926 | 24.641 | 1.00 | 50.00 | C |
| ATOM | 4664 | O | ASN | D | 262 | 23.775 | 30.055 | 24.685 | 1.00 | 50.00 | O |
| ATOM | 4665 | CB | ASN | D | 262 | 23.440 | 27.590 | 26.807 | 1.00 | 50.00 | C |
| ATOM | 4666 | CG | ASN | D | 262 | 22.992 | 27.350 | 28.244 | 1.00 | 50.00 | C |
| ATOM | 4667 | OD1 | ASN | D | 262 | 23.175 | 26.261 | 28.786 | 1.00 | 50.00 | O |
| ATOM | 4668 | ND2 | ASN | D | 262 | 22.415 | 28.321 | 28.928 | 1.00 | 50.00 | N |
| ATOM | 4669 | N | ASP | D | 263 | 23.304 | 28.202 | 23.533 | 1.00 | 50.00 | N |
| ATOM | 4670 | CA | ASP | D | 263 | 23.733 | 28.679 | 22.217 | 1.00 | 50.00 | C |
| ATOM | 4671 | C | ASP | D | 263 | 22.859 | 29.741 | 21.555 | 1.00 | 50.00 | C |
| ATOM | 4672 | O | ASP | D | 263 | 23.387 | 30.770 | 21.132 | 1.00 | 50.00 | O |
| ATOM | 4673 | CB | ASP | D | 263 | 23.935 | 27.493 | 21.272 | 1.00 | 50.00 | C |
| ATOM | 4674 | CG | ASP | D | 263 | 25.007 | 26.482 | 21.652 | 1.00 | 50.00 | C |
| ATOM | 4675 | OD1 | ASP | D | 263 | 26.183 | 26.876 | 21.811 | 1.00 | 50.00 | O |
| ATOM | 4676 | OD2 | ASP | D | 263 | 24.683 | 25.283 | 21.798 | 1.00 | 50.00 | O |
| ATOM | 4677 | N | MET | D | 264 | 21.557 | 29.530 | 21.447 | 1.00 | 50.00 | N |
| ATOM | 4678 | CA | MET | D | 264 | 20.567 | 30.458 | 20.898 | 1.00 | 50.00 | C |
| ATOM | 4679 | C | MET | D | 264 | 20.467 | 31.677 | 21.816 | 1.00 | 50.00 | C |
| ATOM | 4680 | O | MET | D | 264 | 21.059 | 32.684 | 21.428 | 1.00 | 50.00 | O |
| ATOM | 4681 | CB | MET | D | 264 | 19.374 | 29.564 | 20.546 | 1.00 | 50.00 | C |
| ATOM | 4682 | CG | MET | D | 264 | 18.148 | 30.126 | 19.828 | 1.00 | 50.00 | C |
| ATOM | 4683 | SD | MET | D | 264 | 16.702 | 29.053 | 19.899 | 1.00 | 50.00 | S |
| ATOM | 4684 | CE | MET | D | 264 | 16.318 | 28.736 | 21.630 | 1.00 | 50.00 | C |
| ATOM | 4685 | N | PRO | D | 265 | 19.879 | 31.665 | 23.005 | 1.00 | 50.00 | N |
| ATOM | 4686 | CA | PRO | D | 265 | 20.085 | 32.691 | 24.035 | 1.00 | 50.00 | C |
| ATOM | 4687 | C | PRO | D | 265 | 20.829 | 32.355 | 25.330 | 1.00 | 50.00 | C |
| ATOM | 4688 | O | PRO | D | 265 | 20.785 | 31.216 | 25.790 | 1.00 | 50.00 | O |
| ATOM | 4689 | CB | PRO | D | 265 | 18.606 | 33.051 | 24.178 | 1.00 | 50.00 | C |
| ATOM | 4690 | CG | PRO | D | 265 | 17.817 | 31.748 | 24.219 | 1.00 | 50.00 | C |
| ATOM | 4691 | CD | PRO | D | 265 | 18.552 | 31.054 | 23.083 | 1.00 | 50.00 | C |
| ATOM | 4692 | N | ILE | D | 266 | 21.472 | 33.288 | 26.021 | 1.00 | 50.00 | N |
| ATOM | 4693 | CA | ILE | D | 266 | 21.940 | 33.229 | 27.413 | 1.00 | 50.00 | C |
| ATOM | 4694 | C | ILE | D | 266 | 20.858 | 32.972 | 28.468 | 1.00 | 50.00 | C |
| ATOM | 4695 | O | ILE | D | 266 | 19.678 | 32.796 | 28.166 | 1.00 | 50.00 | O |
| ATOM | 4696 | CB | ILE | D | 266 | 22.857 | 34.436 | 27.670 | 1.00 | 50.00 | C |
| ATOM | 4697 | CG1 | ILE | D | 266 | 24.008 | 34.716 | 26.698 | 1.00 | 50.00 | C |
| ATOM | 4698 | CG2 | ILE | D | 266 | 23.465 | 34.556 | 29.070 | 1.00 | 50.00 | C |
| ATOM | 4699 | CD1 | ILE | D | 266 | 24.640 | 36.108 | 26.722 | 1.00 | 50.00 | C |
| ATOM | 4700 | N | THR | D | 267 | 21.138 | 32.858 | 29.759 | 1.00 | 50.00 | N |
| ATOM | 4701 | CA | THR | D | 267 | 20.178 | 32.910 | 30.865 | 1.00 | 50.00 | C |
| ATOM | 4702 | C | THR | D | 267 | 19.467 | 34.238 | 31.107 | 1.00 | 50.00 | C |
| ATOM | 4703 | O | THR | D | 267 | 18.245 | 34.200 | 31.243 | 1.00 | 50.00 | O |
| ATOM | 4704 | CB | THR | D | 267 | 20.791 | 32.424 | 32.181 | 1.00 | 50.00 | C |
| ATOM | 4705 | OG1 | THR | D | 267 | 21.201 | 31.071 | 32.038 | 1.00 | 50.00 | O |
| ATOM | 4706 | CG2 | THR | D | 267 | 19.906 | 32.473 | 33.427 | 1.00 | 50.00 | C |
| ATOM | 4707 | N | ASN | D | 268 | 20.147 | 35.362 | 31.279 | 1.00 | 50.00 | N |
| ATOM | 4708 | CA | ASN | D | 268 | 19.541 | 36.692 | 31.377 | 1.00 | 50.00 | C |
| ATOM | 4709 | C | ASN | D | 268 | 18.406 | 37.048 | 30.422 | 1.00 | 50.00 | C |
| ATOM | 4710 | O | ASN | D | 268 | 17.459 | 37.744 | 30.787 | 1.00 | 50.00 | O |
| ATOM | 4711 | CB | ASN | D | 268 | 20.629 | 37.769 | 31.392 | 1.00 | 50.00 | C |
| ATOM | 4712 | CG | ASN | D | 268 | 21.582 | 38.036 | 30.233 | 1.00 | 50.00 | C |
| ATOM | 4713 | OD1 | ASN | D | 268 | 22.342 | 39.003 | 30.258 | 1.00 | 50.00 | O |
| ATOM | 4714 | ND2 | ASN | D | 268 | 21.610 | 37.247 | 29.175 | 1.00 | 50.00 | N |
| ATOM | 4715 | N | ASP | D | 269 | 18.507 | 36.540 | 29.205 | 1.00 | 50.00 | N |
| ATOM | 4716 | CA | ASP | D | 269 | 17.479 | 36.583 | 28.167 | 1.00 | 50.00 | C |
| ATOM | 4717 | C | ASP | D | 269 | 16.287 | 35.647 | 28.342 | 1.00 | 50.00 | C |
| ATOM | 4718 | O | ASP | D | 269 | 15.283 | 35.766 | 27.644 | 1.00 | 50.00 | O |
| ATOM | 4719 | CB | ASP | D | 269 | 18.218 | 36.618 | 26.826 | 1.00 | 50.00 | C |
| ATOM | 4720 | CG | ASP | D | 269 | 19.464 | 35.822 | 26.498 | 1.00 | 50.00 | C |
| ATOM | 4721 | OD1 | ASP | D | 269 | 20.561 | 36.317 | 26.159 | 1.00 | 50.00 | O |
| ATOM | 4722 | OD2 | ASP | D | 269 | 19.240 | 34.601 | 26.601 | 1.00 | 50.00 | O |
| ATOM | 4723 | N | GLN | D | 270 | 16.199 | 34.802 | 29.355 | 1.00 | 50.00 | N |
| ATOM | 4724 | CA | GLN | D | 270 | 14.983 | 34.066 | 29.706 | 1.00 | 50.00 | C |
| ATOM | 4725 | C | GLN | D | 270 | 13.987 | 34.875 | 30.534 | 1.00 | 50.00 | C |
| ATOM | 4726 | O | GLN | D | 270 | 14.302 | 35.984 | 30.965 | 1.00 | 50.00 | O |
| ATOM | 4727 | CB | GLN | D | 270 | 15.397 | 32.701 | 30.258 | 1.00 | 50.00 | C |
| ATOM | 4728 | CG | GLN | D | 270 | 16.424 | 31.866 | 29.493 | 1.00 | 50.00 | C |
| ATOM | 4729 | CD | GLN | D | 270 | 16.030 | 31.273 | 28.149 | 1.00 | 50.00 | C |
| ATOM | 4730 | OE1 | GLN | D | 270 | 14.902 | 30.885 | 27.851 | 1.00 | 50.00 | O |
| ATOM | 4731 | NE2 | GLN | D | 270 | 17.032 | 31.092 | 27.311 | 1.00 | 50.00 | N |
| ATOM | 4732 | N | LYS | D | 271 | 12.768 | 34.377 | 30.688 | 1.00 | 50.00 | N |
| ATOM | 4733 | CA | LYS | D | 271 | 11.552 | 34.956 | 31.270 | 1.00 | 50.00 | C |
| ATOM | 4734 | C | LYS | D | 271 | 10.352 | 34.239 | 31.940 | 1.00 | 50.00 | C |
| ATOM | 4735 | O | LYS | D | 271 | 10.004 | 34.918 | 32.906 | 1.00 | 50.00 | O |
| ATOM | 4736 | CB | LYS | D | 271 | 11.426 | 36.378 | 30.695 | 1.00 | 50.00 | C |
| ATOM | 4737 | CG | LYS | D | 271 | 11.557 | 37.025 | 29.307 | 1.00 | 50.00 | C |
| ATOM | 4738 | CD | LYS | D | 271 | 12.728 | 37.672 | 28.553 | 1.00 | 50.00 | C |
| ATOM | 4739 | CE | LYS | D | 271 | 13.160 | 39.078 | 28.120 | 1.00 | 50.00 | C |
| ATOM | 4740 | NZ | LYS | D | 271 | 13.992 | 40.063 | 28.827 | 1.00 | 50.00 | N |
| ATOM | 4741 | N | LYS | D | 272 | 9.537 | 33.188 | 32.102 | 1.00 | 50.00 | N |
| ATOM | 4742 | CA | LYS | D | 272 | 8.701 | 32.146 | 31.468 | 1.00 | 50.00 | C |
| ATOM | 4743 | C | LYS | D | 272 | 8.976 | 30.703 | 30.999 | 1.00 | 50.00 | C |
| ATOM | 4744 | O | LYS | D | 272 | 8.443 | 29.822 | 31.670 | 1.00 | 50.00 | O |
| ATOM | 4745 | CB | LYS | D | 272 | 7.255 | 32.482 | 31.065 | 1.00 | 50.00 | C |
| ATOM | 4746 | CG | LYS | D | 272 | 6.223 | 31.514 | 30.468 | 1.00 | 50.00 | C |
| ATOM | 4747 | CD | LYS | D | 272 | 4.767 | 31.445 | 30.935 | 1.00 | 50.00 | C |
| ATOM | 4748 | CE | LYS | D | 272 | 3.936 | 30.253 | 30.457 | 1.00 | 50.00 | C |
| ATOM | 4749 | NZ | LYS | D | 272 | 2.501 | 30.388 | 30.754 | 1.00 | 50.00 | N |
| ATOM | 4750 | N | LEU | D | 273 | 9.426 | 29.945 | 30.003 | 1.00 | 50.00 | N |
| ATOM | 4751 | CA | LEU | D | 273 | 9.772 | 30.146 | 28.590 | 1.00 | 50.00 | C |
| ATOM | 4752 | C | LEU | D | 273 | 10.991 | 29.390 | 28.063 | 1.00 | 50.00 | C |
| ATOM | 4753 | O | LEU | D | 273 | 12.106 | 29.902 | 27.953 | 1.00 | 50.00 | O |
| ATOM | 4754 | CB | LEU | D | 273 | 9.862 | 31.546 | 27.982 | 1.00 | 50.00 | C |
| ATOM | 4755 | CG | LEU | D | 273 | 10.456 | 32.590 | 28.922 | 1.00 | 50.00 | C |
| ATOM | 4756 | CD1 | LEU | D | 273 | 11.550 | 32.047 | 29.852 | 1.00 | 50.00 | C |
| ATOM | 4757 | CD2 | LEU | D | 273 | 9.822 | 33.974 | 28.776 | 1.00 | 50.00 | C |
| ATOM | 4758 | N | MET | D | 274 | 10.852 | 28.178 | 27.553 | 1.00 | 50.00 | N |
| ATOM | 4759 | CA | MET | D | 274 | 10.983 | 27.925 | 26.116 | 1.00 | 50.00 | C |
| ATOM | 4760 | C | MET | D | 274 | 9.774 | 28.307 | 25.267 | 1.00 | 50.00 | C |
| ATOM | 4761 | O | MET | D | 274 | 9.943 | 28.568 | 24.077 | 1.00 | 50.00 | O |
| ATOM | 4762 | CB | MET | D | 274 | 11.321 | 26.451 | 25.882 | 1.00 | 50.00 | C |
| ATOM | 4763 | CG | MET | D | 274 | 12.776 | 26.108 | 25.573 | 1.00 | 50.00 | C |
| ATOM | 4764 | SD | MET | D | 274 | 13.110 | 26.195 | 23.805 | 1.00 | 50.00 | S |
| ATOM | 4765 | CE | MET | D | 274 | 13.479 | 24.477 | 23.408 | 1.00 | 50.00 | C |
| ATOM | 4766 | N | SER | D | 275 | 8.579 | 28.307 | 25.836 | 1.00 | 50.00 | N |
| ATOM | 4767 | CA | SER | D | 275 | 7.299 | 28.602 | 25.191 | 1.00 | 50.00 | C |
| ATOM | 4768 | C | SER | D | 275 | 7.180 | 29.843 | 24.318 | 1.00 | 50.00 | C |
| ATOM | 4769 | O | SER | D | 275 | 6.646 | 29.761 | 23.213 | 1.00 | 50.00 | O |
| ATOM | 4770 | CB | SER | D | 275 | 6.200 | 28.725 | 26.246 | 1.00 | 50.00 | C |
| ATOM | 4771 | OG | SER | D | 275 | 6.381 | 29.801 | 27.155 | 1.00 | 50.00 | O |
| ATOM | 4772 | N | ASN | D | 276 | 7.653 | 30.969 | 24.824 | 1.00 | 50.00 | N |
| ATOM | 4773 | CA | ASN | D | 276 | 7.687 | 32.237 | 24.098 | 1.00 | 50.00 | C |
| ATOM | 4774 | C | ASN | D | 276 | 8.510 | 32.111 | 22.826 | 1.00 | 50.00 | C |
| ATOM | 4775 | O | ASN | D | 276 | 7.891 | 32.163 | 21.765 | 1.00 | 50.00 | O |
| ATOM | 4776 | CB | ASN | D | 276 | 8.128 | 33.352 | 25.044 | 1.00 | 50.00 | C |
| ATOM | 4777 | CG | ASN | D | 276 | 7.116 | 33.596 | 26.154 | 1.00 | 50.00 | C |
| ATOM | 4778 | OD1 | ASN | D | 276 | 7.393 | 33.379 | 27.331 | 1.00 | 50.00 | O |
| ATOM | 4779 | ND2 | ASN | D | 276 | 5.856 | 33.901 | 25.909 | 1.00 | 50.00 | N |
| ATOM | 4780 | N | ASN | D | 277 | 9.778 | 31.746 | 22.917 | 1.00 | 50.00 | N |
| ATOM | 4781 | CA | ASN | D | 277 | 10.586 | 31.266 | 21.795 | 1.00 | 50.00 | C |
| ATOM | 4782 | C | ASN | D | 277 | 9.891 | 30.393 | 20.757 | 1.00 | 50.00 | C |
| ATOM | 4783 | O | ASN | D | 277 | 9.742 | 30.878 | 19.637 | 1.00 | 50.00 | O |
| ATOM | 4784 | CB | ASN | D | 277 | 11.927 | 30.764 | 22.329 | 1.00 | 50.00 | C |
| ATOM | 4785 | CG | ASN | D | 277 | 12.790 | 31.734 | 23.127 | 1.00 | 50.00 | C |
| ATOM | 4786 | OD1 | ASN | D | 277 | 13.555 | 31.303 | 23.989 | 1.00 | 50.00 | O |
| ATOM | 4787 | ND2 | ASN | D | 277 | 12.764 | 33.024 | 22.840 | 1.00 | 50.00 | N |
| ATOM | 4788 | N | VAL | D | 278 | 9.330 | 29.252 | 21.127 | 1.00 | 50.00 | N |
| ATOM | 4789 | CA | VAL | D | 278 | 8.419 | 28.443 | 20.312 | 1.00 | 50.00 | C |
| ATOM | 4790 | C | VAL | D | 278 | 7.303 | 29.207 | 19.605 | 1.00 | 50.00 | C |
| ATOM | 4791 | O | VAL | D | 278 | 7.200 | 29.161 | 18.379 | 1.00 | 50.00 | O |
| ATOM | 4792 | CB | VAL | D | 278 | 7.918 | 27.243 | 21.123 | 1.00 | 50.00 | C |
| ATOM | 4793 | CG1 | VAL | D | 278 | 6.848 | 26.399 | 20.432 | 1.00 | 50.00 | C |
| ATOM | 4794 | CG2 | VAL | D | 278 | 9.025 | 26.288 | 21.568 | 1.00 | 50.00 | C |
| ATOM | 4795 | N | GLN | D | 279 | 6.446 | 29.885 | 20.350 | 1.00 | 50.00 | N |
| ATOM | 4796 | CA | GLN | D | 279 | 5.353 | 30.697 | 19.816 | 1.00 | 50.00 | C |
| ATOM | 4797 | C | GLN | D | 279 | 5.712 | 31.930 | 18.994 | 1.00 | 50.00 | C |
| ATOM | 4798 | O | GLN | D | 279 | 4.864 | 32.357 | 18.211 | 1.00 | 50.00 | O |
| ATOM | 4799 | CB | GLN | D | 279 | 4.426 | 31.047 | 20.981 | 1.00 | 50.00 | C |
| ATOM | 4800 | CG | GLN | D | 279 | 3.592 | 29.923 | 21.594 | 1.00 | 50.00 | C |
| ATOM | 4801 | CD | GLN | D | 279 | 2.616 | 29.273 | 20.624 | 1.00 | 50.00 | C |
| ATOM | 4802 | OE1 | GLN | D | 279 | 2.091 | 29.877 | 19.689 | 1.00 | 50.00 | O |
| ATOM | 4803 | NE2 | GLN | D | 279 | 2.357 | 27.996 | 20.828 | 1.00 | 50.00 | N |
| ATOM | 4804 | N | ILE | D | 280 | 6.902 | 32.498 | 19.112 | 1.00 | 50.00 | N |
| ATOM | 4805 | CA | ILE | D | 280 | 7.401 | 33.608 | 18.296 | 1.00 | 50.00 | C |
| ATOM | 4806 | C | ILE | D | 280 | 7.344 | 33.387 | 16.791 | 1.00 | 50.00 | C |
| ATOM | 4807 | O | ILE | D | 280 | 7.174 | 34.387 | 16.095 | 1.00 | 50.00 | O |
| ATOM | 4808 | CB | ILE | D | 280 | 8.787 | 34.085 | 18.743 | 1.00 | 50.00 | C |
| ATOM | 4809 | CG1 | ILE | D | 280 | 8.629 | 34.747 | 20.110 | 1.00 | 50.00 | C |
| ATOM | 4810 | CG2 | ILE | D | 280 | 9.509 | 35.113 | 17.872 | 1.00 | 50.00 | C |
| ATOM | 4811 | CD1 | ILE | D | 280 | 9.818 | 34.905 | 21.047 | 1.00 | 50.00 | C |
| ATOM | 4812 | N | VAL | D | 281 | 7.514 | 32.165 | 16.311 | 1.00 | 50.00 | N |
| ATOM | 4813 | CA | VAL | D | 281 | 7.633 | 31.853 | 14.885 | 1.00 | 50.00 | C |
| ATOM | 4814 | C | VAL | D | 281 | 9.018 | 32.151 | 14.311 | 1.00 | 50.00 | C |
| ATOM | 4815 | O | VAL | D | 281 | 9.212 | 32.127 | 13.096 | 1.00 | 50.00 | O |
| ATOM | 4816 | CB | VAL | D | 281 | 6.350 | 32.276 | 14.159 | 1.00 | 50.00 | C |
| ATOM | 4817 | CG1 | VAL | D | 281 | 6.326 | 32.275 | 12.632 | 1.00 | 50.00 | C |
| ATOM | 4818 | CG2 | VAL | D | 281 | 5.089 | 31.582 | 14.678 | 1.00 | 50.00 | C |
| ATOM | 4819 | N | ARG | D | 282 | 10.040 | 32.347 | 15.129 | 1.00 | 50.00 | N |
| ATOM | 4820 | CA | ARG | D | 282 | 11.440 | 32.555 | 14.758 | 1.00 | 50.00 | C |
| ATOM | 4821 | C | ARG | D | 282 | 12.211 | 31.329 | 14.276 | 1.00 | 50.00 | C |
| ATOM | 4822 | O | ARG | D | 282 | 12.945 | 31.423 | 13.293 | 1.00 | 50.00 | O |
| ATOM | 4823 | CB | ARG | D | 282 | 12.100 | 33.242 | 15.955 | 1.00 | 50.00 | C |
| ATOM | 4824 | CG | ARG | D | 282 | 12.190 | 32.405 | 17.233 | 1.00 | 50.00 | C |
| ATOM | 4825 | CD | ARG | D | 282 | 12.835 | 33.100 | 18.428 | 1.00 | 50.00 | C |
| ATOM | 4826 | NE | ARG | D | 282 | 13.447 | 32.169 | 19.377 | 1.00 | 50.00 | N |
| ATOM | 4827 | CZ | ARG | D | 282 | 14.492 | 32.410 | 20.153 | 1.00 | 50.00 | C |
| ATOM | 4828 | NH1 | ARG | D | 282 | 15.157 | 33.547 | 20.244 | 1.00 | 50.00 | N |
| ATOM | 4829 | NH2 | ARG | D | 282 | 14.932 | 31.427 | 20.913 | 1.00 | 50.00 | N |
| ATOM | 4830 | N | GLN | D | 283 | 12.068 | 30.204 | 14.958 | 1.00 | 50.00 | N |
| ATOM | 4831 | CA | GLN | D | 283 | 12.721 | 28.925 | 14.679 | 1.00 | 50.00 | C |
| ATOM | 4832 | C | GLN | D | 283 | 11.940 | 28.199 | 13.591 | 1.00 | 50.00 | C |
| ATOM | 4833 | O | GLN | D | 283 | 10.968 | 27.495 | 13.864 | 1.00 | 50.00 | O |
| ATOM | 4834 | CB | GLN | D | 283 | 12.900 | 28.097 | 15.954 | 1.00 | 50.00 | C |
| ATOM | 4835 | CG | GLN | D | 283 | 11.700 | 27.712 | 16.820 | 1.00 | 50.00 | C |
| ATOM | 4836 | CD | GLN | D | 283 | 11.118 | 28.878 | 17.605 | 1.00 | 50.00 | C |
| ATOM | 4837 | OE1 | GLN | D | 283 | 11.785 | 29.453 | 18.464 | 1.00 | 50.00 | O |
| ATOM | 4838 | NE2 | GLN | D | 283 | 9.901 | 29.278 | 17.286 | 1.00 | 50.00 | N |
| ATOM | 4839 | N | GLN | D | 284 | 12.330 | 28.419 | 12.346 | 1.00 | 50.00 | N |
| ATOM | 4840 | CA | GLN | D | 284 | 11.609 | 27.904 | 11.183 | 1.00 | 50.00 | C |
| ATOM | 4841 | C | GLN | D | 284 | 12.517 | 27.056 | 10.304 | 1.00 | 50.00 | C |
| ATOM | 4842 | O | GLN | D | 284 | 13.730 | 27.249 | 10.250 | 1.00 | 50.00 | O |
| ATOM | 4843 | CB | GLN | D | 284 | 10.943 | 29.029 | 10.389 | 1.00 | 50.00 | C |
| ATOM | 4844 | CG | GLN | D | 284 | 9.686 | 29.606 | 11.041 | 1.00 | 50.00 | C |
| ATOM | 4845 | CD | GLN | D | 284 | 8.513 | 28.648 | 11.198 | 1.00 | 50.00 | C |
| ATOM | 4846 | OE1 | GLN | D | 284 | 8.434 | 27.571 | 10.609 | 1.00 | 50.00 | O |
| ATOM | 4847 | NE2 | GLN | D | 284 | 7.548 | 29.012 | 12.022 | 1.00 | 50.00 | N |
| ATOM | 4848 | N | SER | D | 285 | 11.893 | 26.109 | 9.622 | 1.00 | 50.00 | N |
| ATOM | 4849 | CA | SER | D | 285 | 12.585 | 25.086 | 8.839 | 1.00 | 50.00 | C |
| ATOM | 4850 | C | SER | D | 285 | 11.876 | 24.822 | 7.518 | 1.00 | 50.00 | C |
| ATOM | 4851 | O | SER | D | 285 | 10.658 | 24.965 | 7.407 | 1.00 | 50.00 | O |
| ATOM | 4852 | CB | SER | D | 285 | 12.710 | 23.779 | 9.620 | 1.00 | 50.00 | C |
| ATOM | 4853 | OG | SER | D | 285 | 13.245 | 23.969 | 10.922 | 1.00 | 50.00 | O |
| ATOM | 4854 | N | TYR | D | 286 | 12.646 | 24.424 | 6.518 | 1.00 | 50.00 | N |
| ATOM | 4855 | CA | TYR | D | 286 | 12.144 | 24.213 | 5.160 | 1.00 | 50.00 | C |
| ATOM | 4856 | C | TYR | D | 286 | 12.901 | 23.066 | 4.507 | 1.00 | 50.00 | C |
| ATOM | 4857 | O | TYR | D | 286 | 14.131 | 23.043 | 4.520 | 1.00 | 50.00 | O |
| ATOM | 4858 | CB | TYR | D | 286 | 12.252 | 25.475 | 4.300 | 1.00 | 50.00 | C |
| ATOM | 4859 | CG | TYR | D | 286 | 11.253 | 26.546 | 4.681 | 1.00 | 50.00 | C |
| ATOM | 4860 | CD1 | TYR | D | 286 | 9.941 | 26.304 | 4.482 | 1.00 | 50.00 | C |
| ATOM | 4861 | CD2 | TYR | D | 286 | 11.631 | 27.629 | 5.392 | 1.00 | 50.00 | C |
| ATOM | 4862 | CE1 | TYR | D | 286 | 9.012 | 27.098 | 5.051 | 1.00 | 50.00 | C |
| ATOM | 4863 | CE2 | TYR | D | 286 | 10.704 | 28.456 | 5.914 | 1.00 | 50.00 | C |
| ATOM | 4864 | CZ | TYR | D | 286 | 9.393 | 28.192 | 5.741 | 1.00 | 50.00 | C |
| ATOM | 4865 | OH | TYR | D | 286 | 8.443 | 29.070 | 6.192 | 1.00 | 50.00 | O |
| ATOM | 4866 | N | SER | D | 287 | 12.182 | 22.203 | 3.808 | 1.00 | 50.00 | N |
| ATOM | 4867 | CA | SER | D | 287 | 12.770 | 21.246 | 2.871 | 1.00 | 50.00 | C |
| ATOM | 4868 | C | SER | D | 287 | 13.669 | 21.927 | 1.844 | 1.00 | 50.00 | C |
| ATOM | 4869 | O | SER | D | 287 | 13.384 | 23.035 | 1.390 | 1.00 | 50.00 | O |
| ATOM | 4870 | CB | SER | D | 287 | 11.663 | 20.429 | 2.204 | 1.00 | 50.00 | C |
| ATOM | 4871 | OG | SER | D | 287 | 10.979 | 19.606 | 3.137 | 1.00 | 50.00 | O |
| ATOM | 4872 | N | ILE | D | 288 | 14.801 | 21.303 | 1.567 | 1.00 | 50.00 | N |
| ATOM | 4873 | CA | ILE | D | 288 | 15.774 | 21.695 | 0.544 | 1.00 | 50.00 | C |
| ATOM | 4874 | C | ILE | D | 288 | 16.108 | 20.582 | −0.446 | 1.00 | 50.00 | C |
| ATOM | 4875 | O | ILE | D | 288 | 16.505 | 20.895 | −1.568 | 1.00 | 50.00 | O |
| ATOM | 4876 | CB | ILE | D | 288 | 17.044 | 22.338 | 1.108 | 1.00 | 50.00 | C |
| ATOM | 4877 | CG1 | ILE | D | 288 | 17.774 | 21.459 | 2.125 | 1.00 | 50.00 | C |
| ATOM | 4878 | CG2 | ILE | D | 288 | 16.790 | 23.758 | 1.615 | 1.00 | 50.00 | C |
| ATOM | 4879 | CD1 | ILE | D | 288 | 19.280 | 21.667 | 2.263 | 1.00 | 50.00 | C |
| ATOM | 4880 | N | MET | D | 289 | 16.045 | 19.317 | −0.060 | 1.00 | 50.00 | N |
| ATOM | 4881 | CA | MET | D | 289 | 16.567 | 18.178 | −0.814 | 1.00 | 50.00 | C |
| ATOM | 4882 | C | MET | D | 289 | 15.627 | 17.003 | −0.584 | 1.00 | 50.00 | C |
| ATOM | 4883 | O | MET | D | 289 | 15.218 | 16.739 | 0.546 | 1.00 | 50.00 | O |
| ATOM | 4884 | CB | MET | D | 289 | 17.955 | 17.734 | −0.351 | 1.00 | 50.00 | C |
| ATOM | 4885 | CG | MET | D | 289 | 19.096 | 18.730 | −0.552 | 1.00 | 50.00 | C |
| ATOM | 4886 | SD | MET | D | 289 | 19.389 | 18.986 | −2.310 | 1.00 | 50.00 | S |
| ATOM | 4887 | CE | MET | D | 289 | 20.169 | 17.429 | −2.767 | 1.00 | 50.00 | C |
| ATOM | 4888 | N | SER | D | 290 | 15.351 | 16.265 | −1.645 | 1.00 | 50.00 | N |
| ATOM | 4889 | CA | SER | D | 290 | 14.561 | 15.036 | −1.596 | 1.00 | 50.00 | C |
| ATOM | 4890 | C | SER | D | 290 | 15.237 | 14.061 | −2.552 | 1.00 | 50.00 | C |
| ATOM | 4891 | O | SER | D | 290 | 15.000 | 14.125 | −3.758 | 1.00 | 50.00 | O |
| ATOM | 4892 | CB | SER | D | 290 | 13.118 | 15.314 | −2.020 | 1.00 | 50.00 | C |
| ATOM | 4893 | OG | SER | D | 290 | 12.389 | 16.161 | −1.145 | 1.00 | 50.00 | O |
| ATOM | 4894 | N | ILE | D | 291 | 16.049 | 13.142 | −2.052 | 1.00 | 50.00 | N |
| ATOM | 4895 | CA | ILE | D | 291 | 16.607 | 12.044 | −2.845 | 1.00 | 50.00 | C |
| ATOM | 4896 | C | ILE | D | 291 | 15.490 | 11.012 | −2.962 | 1.00 | 50.00 | C |
| ATOM | 4897 | O | ILE | D | 291 | 15.204 | 10.288 | −2.009 | 1.00 | 50.00 | O |
| ATOM | 4898 | CB | ILE | D | 291 | 17.922 | 11.471 | −2.306 | 1.00 | 50.00 | C |
| ATOM | 4899 | CG1 | ILE | D | 291 | 19.046 | 12.485 | −2.078 | 1.00 | 50.00 | C |
| ATOM | 4900 | CG2 | ILE | D | 291 | 18.459 | 10.330 | −3.170 | 1.00 | 50.00 | C |
| ATOM | 4901 | CD1 | ILE | D | 291 | 20.189 | 12.047 | −1.165 | 1.00 | 50.00 | C |
| ATOM | 4902 | N | ILE | D | 292 | 14.872 | 10.934 | −4.130 | 1.00 | 50.00 | N |
| ATOM | 4903 | CA | ILE | D | 292 | 13.855 | 9.939 | −4.477 | 1.00 | 50.00 | C |
| ATOM | 4904 | C | ILE | D | 292 | 14.316 | 8.493 | −4.308 | 1.00 | 50.00 | C |
| ATOM | 4905 | O | ILE | D | 292 | 13.574 | 7.682 | −3.756 | 1.00 | 50.00 | O |
| ATOM | 4906 | CB | ILE | D | 292 | 13.241 | 10.281 | −5.840 | 1.00 | 50.00 | C |
| ATOM | 4907 | CG1 | ILE | D | 292 | 12.404 | 11.563 | −5.819 | 1.00 | 50.00 | C |
| ATOM | 4908 | CG2 | ILE | D | 292 | 12.438 | 9.133 | −6.454 | 1.00 | 50.00 | C |
| ATOM | 4909 | CD1 | ILE | D | 292 | 11.919 | 12.114 | −7.158 | 1.00 | 50.00 | C |
| ATOM | 4910 | N | LYS | D | 293 | 15.489 | 8.147 | −4.812 | 1.00 | 50.00 | N |
| ATOM | 4911 | CA | LYS | D | 293 | 16.010 | 6.780 | −4.864 | 1.00 | 50.00 | C |
| ATOM | 4912 | C | LYS | D | 293 | 16.431 | 6.202 | −3.518 | 1.00 | 50.00 | C |
| ATOM | 4913 | O | LYS | D | 293 | 15.895 | 5.172 | −3.109 | 1.00 | 50.00 | O |
| ATOM | 4914 | CB | LYS | D | 293 | 17.162 | 6.748 | −5.869 | 1.00 | 50.00 | C |
| ATOM | 4915 | CG | LYS | D | 293 | 16.759 | 6.992 | −7.322 | 1.00 | 50.00 | C |
| ATOM | 4916 | CD | LYS | D | 293 | 17.959 | 6.908 | −8.262 | 1.00 | 50.00 | C |
| ATOM | 4917 | CE | LYS | D | 293 | 17.561 | 7.206 | −9.706 | 1.00 | 50.00 | C |
| ATOM | 4918 | NZ | LYS | D | 293 | 18.717 | 7.041 | −10.598 | 1.00 | 50.00 | N |
| ATOM | 4919 | N | GLU | D | 294 | 17.349 | 6.856 | −2.824 | 1.00 | 50.00 | N |
| ATOM | 4920 | CA | GLU | D | 294 | 17.705 | 6.550 | −1.438 | 1.00 | 50.00 | C |
| ATOM | 4921 | C | GLU | D | 294 | 16.635 | 6.777 | −0.376 | 1.00 | 50.00 | C |
| ATOM | 4922 | O | GLU | D | 294 | 16.767 | 6.218 | 0.712 | 1.00 | 50.00 | O |
| ATOM | 4923 | CB | GLU | D | 294 | 18.962 | 7.331 | −1.051 | 1.00 | 50.00 | C |
| ATOM | 4924 | CG | GLU | D | 294 | 20.240 | 7.016 | −1.827 | 1.00 | 50.00 | C |
| ATOM | 4925 | CD | GLU | D | 294 | 20.787 | 5.628 | −1.530 | 1.00 | 50.00 | C |
| ATOM | 4926 | OE1 | GLU | D | 294 | 20.359 | 4.656 | −2.188 | 1.00 | 50.00 | O |
| ATOM | 4927 | OE2 | GLU | D | 294 | 21.632 | 5.495 | −0.618 | 1.00 | 50.00 | O |
| ATOM | 4928 | N | GLU | D | 295 | 15.617 | 7.580 | −0.640 | 1.00 | 50.00 | N |
| ATOM | 4929 | CA | GLU | D | 295 | 14.556 | 7.928 | 0.304 | 1.00 | 50.00 | C |
| ATOM | 4930 | C | GLU | D | 295 | 15.119 | 8.759 | 1.452 | 1.00 | 50.00 | C |
| ATOM | 4931 | O | GLU | D | 295 | 15.246 | 8.280 | 2.578 | 1.00 | 50.00 | O |
| ATOM | 4932 | CB | GLU | D | 295 | 13.685 | 6.715 | 0.643 | 1.00 | 50.00 | C |
| ATOM | 4933 | CG | GLU | D | 295 | 12.963 | 6.004 | −0.502 | 1.00 | 50.00 | C |
| ATOM | 4934 | CD | GLU | D | 295 | 11.730 | 6.771 | −0.952 | 1.00 | 50.00 | C |
| ATOM | 4935 | OE1 | GLU | D | 295 | 11.910 | 7.879 | −1.500 | 1.00 | 50.00 | O |
| ATOM | 4936 | OE2 | GLU | D | 295 | 10.590 | 6.306 | −0.731 | 1.00 | 50.00 | O |
| ATOM | 4937 | N | VAL | D | 296 | 15.507 | 9.987 | 1.143 | 1.00 | 50.00 | N |
| ATOM | 4938 | CA | VAL | D | 296 | 16.212 | 10.903 | 2.040 | 1.00 | 50.00 | C |
| ATOM | 4939 | C | VAL | D | 296 | 15.541 | 12.262 | 1.881 | 1.00 | 50.00 | C |
| ATOM | 4940 | O | VAL | D | 296 | 15.189 | 12.683 | 0.780 | 1.00 | 50.00 | O |
| ATOM | 4941 | CB | VAL | D | 296 | 17.716 | 10.972 | 1.758 | 1.00 | 50.00 | C |
| ATOM | 4942 | CG1 | VAL | D | 296 | 18.500 | 12.036 | 2.528 | 1.00 | 50.00 | C |
| ATOM | 4943 | CG2 | VAL | D | 296 | 18.421 | 9.627 | 1.930 | 1.00 | 50.00 | C |
| ATOM | 4944 | N | LEU | D | 297 | 15.454 | 12.996 | 2.978 | 1.00 | 50.00 | N |
| ATOM | 4945 | CA | LEU | D | 297 | 15.063 | 14.405 | 2.972 | 1.00 | 50.00 | C |
| ATOM | 4946 | C | LEU | D | 297 | 16.031 | 15.235 | 3.806 | 1.00 | 50.00 | C |
| ATOM | 4947 | O | LEU | D | 297 | 16.185 | 14.964 | 4.996 | 1.00 | 50.00 | O |
| ATOM | 4948 | CB | LEU | D | 297 | 13.631 | 14.499 | 3.500 | 1.00 | 50.00 | C |
| ATOM | 4949 | CG | LEU | D | 297 | 12.803 | 15.730 | 3.132 | 1.00 | 50.00 | C |
| ATOM | 4950 | CD1 | LEU | D | 297 | 13.449 | 17.067 | 3.489 | 1.00 | 50.00 | C |
| ATOM | 4951 | CD2 | LEU | D | 297 | 12.271 | 15.816 | 1.706 | 1.00 | 50.00 | C |
| ATOM | 4952 | N | ALA | D | 298 | 16.582 | 16.310 | 3.266 | 1.00 | 50.00 | N |
| ATOM | 4953 | CA | ALA | D | 298 | 17.209 | 17.368 | 4.059 | 1.00 | 50.00 | C |
| ATOM | 4954 | C | ALA | D | 298 | 16.279 | 18.546 | 4.329 | 1.00 | 50.00 | C |
| ATOM | 4955 | O | ALA | D | 298 | 15.862 | 19.224 | 3.391 | 1.00 | 50.00 | O |
| ATOM | 4956 | CB | ALA | D | 298 | 18.460 | 17.860 | 3.332 | 1.00 | 50.00 | C |
| ATOM | 4957 | N | TYR | D | 299 | 15.979 | 18.836 | 5.585 | 1.00 | 50.00 | N |
| ATOM | 4958 | CA | TYR | D | 299 | 15.459 | 20.133 | 6.024 | 1.00 | 50.00 | C |
| ATOM | 4959 | C | TYR | D | 299 | 16.551 | 21.198 | 6.079 | 1.00 | 50.00 | C |
| ATOM | 4960 | O | TYR | D | 299 | 17.738 | 20.873 | 6.049 | 1.00 | 50.00 | O |
| ATOM | 4961 | CB | TYR | D | 299 | 14.587 | 19.954 | 7.272 | 1.00 | 50.00 | C |
| ATOM | 4962 | CG | TYR | D | 299 | 14.896 | 20.380 | 8.692 | 1.00 | 50.00 | C |
| ATOM | 4963 | CD1 | TYR | D | 299 | 16.162 | 20.655 | 9.066 | 1.00 | 50.00 | C |
| ATOM | 4964 | CD2 | TYR | D | 299 | 13.910 | 20.491 | 9.607 | 1.00 | 50.00 | C |
| ATOM | 4965 | CE1 | TYR | D | 299 | 16.442 | 21.090 | 10.309 | 1.00 | 50.00 | C |
| ATOM | 4966 | CE2 | TYR | D | 299 | 14.179 | 20.900 | 10.864 | 1.00 | 50.00 | C |
| ATOM | 4967 | CZ | TYR | D | 299 | 15.450 | 21.175 | 11.217 | 1.00 | 50.00 | C |
| ATOM | 4968 | OH | TYR | D | 299 | 15.770 | 21.523 | 12.503 | 1.00 | 50.00 | O |
| ATOM | 4969 | N | VAL | D | 300 | 16.199 | 22.454 | 6.304 | 1.00 | 50.00 | N |
| ATOM | 4970 | CA | VAL | D | 300 | 17.108 | 23.488 | 6.805 | 1.00 | 50.00 | C |
| ATOM | 4971 | C | VAL | D | 300 | 16.326 | 24.309 | 7.825 | 1.00 | 50.00 | C |
| ATOM | 4972 | O | VAL | D | 300 | 15.554 | 25.184 | 7.437 | 1.00 | 50.00 | O |
| ATOM | 4973 | CB | VAL | D | 300 | 17.701 | 24.369 | 5.702 | 1.00 | 50.00 | C |
| ATOM | 4974 | CG1 | VAL | D | 300 | 18.421 | 25.644 | 6.142 | 1.00 | 50.00 | C |
| ATOM | 4975 | CG2 | VAL | D | 300 | 18.729 | 23.650 | 4.832 | 1.00 | 50.00 | C |
| ATOM | 4976 | N | VAL | D | 301 | 16.633 | 24.145 | 9.102 | 1.00 | 50.00 | N |
| ATOM | 4977 | CA | VAL | D | 301 | 16.304 | 25.101 | 10.161 | 1.00 | 50.00 | C |
| ATOM | 4978 | C | VAL | D | 301 | 17.134 | 26.377 | 10.085 | 1.00 | 50.00 | C |
| ATOM | 4979 | O | VAL | D | 301 | 18.218 | 26.450 | 9.507 | 1.00 | 50.00 | O |
| ATOM | 4980 | CB | VAL | D | 301 | 16.377 | 24.455 | 11.546 | 1.00 | 50.00 | C |
| ATOM | 4981 | CG1 | VAL | D | 301 | 17.798 | 24.015 | 11.891 | 1.00 | 50.00 | C |
| ATOM | 4982 | CG2 | VAL | D | 301 | 15.718 | 25.124 | 12.752 | 1.00 | 50.00 | C |
| ATOM | 4983 | N | GLN | D | 302 | 16.578 | 27.378 | 10.744 | 1.00 | 50.00 | N |
| ATOM | 4984 | CA | GLN | D | 302 | 17.134 | 28.725 | 10.838 | 1.00 | 50.00 | C |
| ATOM | 4985 | C | GLN | D | 302 | 16.803 | 29.190 | 12.249 | 1.00 | 50.00 | C |
| ATOM | 4986 | O | GLN | D | 302 | 15.684 | 29.634 | 12.505 | 1.00 | 50.00 | O |
| ATOM | 4987 | CB | GLN | D | 302 | 16.498 | 29.587 | 9.746 | 1.00 | 50.00 | C |
| ATOM | 4988 | CG | GLN | D | 302 | 16.972 | 29.296 | 8.323 | 1.00 | 50.00 | C |
| ATOM | 4989 | CD | GLN | D | 302 | 18.446 | 29.568 | 8.056 | 1.00 | 50.00 | C |
| ATOM | 4990 | OE1 | GLN | D | 302 | 19.156 | 30.218 | 8.822 | 1.00 | 50.00 | O |
| ATOM | 4991 | NE2 | GLN | D | 302 | 18.961 | 29.067 | 6.948 | 1.00 | 50.00 | N |
| ATOM | 4992 | N | LEU | D | 303 | 17.755 | 29.062 | 13.160 | 1.00 | 50.00 | N |
| ATOM | 4993 | CA | LEU | D | 303 | 17.566 | 29.481 | 14.548 | 1.00 | 50.00 | C |
| ATOM | 4994 | C | LEU | D | 303 | 17.982 | 30.932 | 14.771 | 1.00 | 50.00 | C |
| ATOM | 4995 | O | LEU | D | 303 | 18.951 | 31.389 | 14.166 | 1.00 | 50.00 | O |
| ATOM | 4996 | CB | LEU | D | 303 | 18.135 | 28.599 | 15.659 | 1.00 | 50.00 | C |
| ATOM | 4997 | CG | LEU | D | 303 | 17.571 | 27.182 | 15.749 | 1.00 | 50.00 | C |
| ATOM | 4998 | CD1 | LEU | D | 303 | 18.209 | 26.202 | 14.767 | 1.00 | 50.00 | C |
| ATOM | 4999 | CD2 | LEU | D | 303 | 17.759 | 26.639 | 17.163 | 1.00 | 50.00 | C |
| ATOM | 5000 | N | PRO | D | 304 | 17.322 | 31.676 | 15.645 | 1.00 | 50.00 | N |
| ATOM | 5001 | CA | PRO | D | 304 | 17.695 | 33.045 | 16.003 | 1.00 | 50.00 | C |
| ATOM | 5002 | C | PRO | D | 304 | 18.612 | 33.160 | 17.215 | 1.00 | 50.00 | C |
| ATOM | 5003 | O | PRO | D | 304 | 18.945 | 32.158 | 17.847 | 1.00 | 50.00 | O |
| ATOM | 5004 | CB | PRO | D | 304 | 16.338 | 33.695 | 16.259 | 1.00 | 50.00 | C |
| ATOM | 5005 | CG | PRO | D | 304 | 15.613 | 32.528 | 16.919 | 1.00 | 50.00 | C |
| ATOM | 5006 | CD | PRO | D | 304 | 16.107 | 31.220 | 16.318 | 1.00 | 50.00 | C |
| ATOM | 5007 | N | LEU | D | 305 | 19.117 | 34.347 | 17.503 | 1.00 | 50.00 | N |
| ATOM | 5008 | CA | LEU | D | 305 | 20.152 | 34.527 | 18.521 | 1.00 | 50.00 | C |
| ATOM | 5009 | C | LEU | D | 305 | 19.737 | 35.723 | 19.360 | 1.00 | 50.00 | C |
| ATOM | 5010 | O | LEU | D | 305 | 20.035 | 36.886 | 19.087 | 1.00 | 50.00 | O |
| ATOM | 5011 | CB | LEU | D | 305 | 21.561 | 34.787 | 17.997 | 1.00 | 50.00 | C |
| ATOM | 5012 | CG | LEU | D | 305 | 22.272 | 33.551 | 17.452 | 1.00 | 50.00 | C |
| ATOM | 5013 | CD1 | LEU | D | 305 | 21.636 | 33.153 | 16.123 | 1.00 | 50.00 | C |
| ATOM | 5014 | CD2 | LEU | D | 305 | 23.760 | 33.879 | 17.355 | 1.00 | 50.00 | C |
| ATOM | 5015 | N | TYR | D | 306 | 19.062 | 35.336 | 20.426 | 1.00 | 50.00 | N |
| ATOM | 5016 | CA | TYR | D | 306 | 18.481 | 36.306 | 21.346 | 1.00 | 50.00 | C |
| ATOM | 5017 | C | TYR | D | 306 | 19.506 | 36.746 | 22.383 | 1.00 | 50.00 | C |
| ATOM | 5018 | O | TYR | D | 306 | 19.992 | 35.982 | 23.216 | 1.00 | 50.00 | O |
| ATOM | 5019 | CB | TYR | D | 306 | 17.166 | 35.675 | 21.795 | 1.00 | 50.00 | C |
| ATOM | 5020 | CG | TYR | D | 306 | 16.456 | 36.431 | 22.891 | 1.00 | 50.00 | C |
| ATOM | 5021 | CD1 | TYR | D | 306 | 15.899 | 37.610 | 22.553 | 1.00 | 50.00 | C |
| ATOM | 5022 | CD2 | TYR | D | 306 | 16.133 | 35.855 | 24.066 | 1.00 | 50.00 | C |
| ATOM | 5023 | CE1 | TYR | D | 306 | 14.770 | 37.987 | 23.180 | 1.00 | 50.00 | C |
| ATOM | 5024 | CE2 | TYR | D | 306 | 15.211 | 36.455 | 24.846 | 1.00 | 50.00 | C |
| ATOM | 5025 | CZ | TYR | D | 306 | 14.435 | 37.442 | 24.363 | 1.00 | 50.00 | C |
| ATOM | 5026 | OH | TYR | D | 306 | 13.589 | 38.161 | 25.162 | 1.00 | 50.00 | O |
| ATOM | 5027 | N | GLY | D | 307 | 19.794 | 38.029 | 22.258 | 1.00 | 50.00 | N |
| ATOM | 5028 | CA | GLY | D | 307 | 20.578 | 38.797 | 23.220 | 1.00 | 50.00 | C |
| ATOM | 5029 | C | GLY | D | 307 | 19.640 | 39.821 | 23.840 | 1.00 | 50.00 | C |
| ATOM | 5030 | O | GLY | D | 307 | 19.317 | 40.840 | 23.230 | 1.00 | 50.00 | O |
| ATOM | 5031 | N | VAL | D | 308 | 19.248 | 39.540 | 25.070 | 1.00 | 50.00 | N |
| ATOM | 5032 | CA | VAL | D | 308 | 18.477 | 40.448 | 25.919 | 1.00 | 50.00 | C |
| ATOM | 5033 | C | VAL | D | 308 | 19.061 | 41.841 | 26.098 | 1.00 | 50.00 | C |
| ATOM | 5034 | O | VAL | D | 308 | 20.271 | 42.060 | 26.138 | 1.00 | 50.00 | O |
| ATOM | 5035 | CB | VAL | D | 308 | 18.172 | 39.845 | 27.290 | 1.00 | 50.00 | C |
| ATOM | 5036 | CG1 | VAL | D | 308 | 19.424 | 39.554 | 28.117 | 1.00 | 50.00 | C |
| ATOM | 5037 | CG2 | VAL | D | 308 | 16.992 | 40.284 | 28.152 | 1.00 | 50.00 | C |
| ATOM | 5038 | N | ILE | D | 309 | 18.113 | 42.709 | 26.400 | 1.00 | 50.00 | N |
| ATOM | 5039 | CA | ILE | D | 309 | 18.375 | 43.918 | 27.177 | 1.00 | 50.00 | C |
| ATOM | 5040 | C | ILE | D | 309 | 18.243 | 43.527 | 28.645 | 1.00 | 50.00 | C |
| ATOM | 5041 | O | ILE | D | 309 | 17.161 | 43.615 | 29.226 | 1.00 | 50.00 | O |
| ATOM | 5042 | CB | ILE | D | 309 | 17.459 | 45.043 | 26.692 | 1.00 | 50.00 | C |
| ATOM | 5043 | CG1 | ILE | D | 309 | 17.558 | 45.282 | 25.185 | 1.00 | 50.00 | C |
| ATOM | 5044 | CG2 | ILE | D | 309 | 17.804 | 46.331 | 27.437 | 1.00 | 50.00 | C |
| ATOM | 5045 | CD1 | ILE | D | 309 | 16.535 | 46.242 | 24.586 | 1.00 | 50.00 | C |
| ATOM | 5046 | N | ASP | D | 310 | 19.352 | 43.080 | 29.212 | 1.00 | 50.00 | N |
| ATOM | 5047 | CA | ASP | D | 310 | 19.519 | 42.760 | 30.631 | 1.00 | 50.00 | C |
| ATOM | 5048 | C | ASP | D | 310 | 19.613 | 44.000 | 31.513 | 1.00 | 50.00 | C |
| ATOM | 5049 | O | ASP | D | 310 | 20.633 | 44.395 | 32.075 | 1.00 | 50.00 | O |
| ATOM | 5050 | CB | ASP | D | 310 | 20.701 | 41.812 | 30.840 | 1.00 | 50.00 | C |
| ATOM | 5051 | CG | ASP | D | 310 | 22.045 | 42.229 | 30.258 | 1.00 | 50.00 | C |
| ATOM | 5052 | OD1 | ASP | D | 310 | 22.281 | 41.982 | 29.056 | 1.00 | 50.00 | O |
| ATOM | 5053 | OD2 | ASP | D | 310 | 22.862 | 42.836 | 30.984 | 1.00 | 50.00 | O |
| ATOM | 5054 | N | THR | D | 311 | 18.447 | 44.612 | 31.615 | 1.00 | 50.00 | N |
| ATOM | 5055 | CA | THR | D | 311 | 18.112 | 45.741 | 32.479 | 1.00 | 50.00 | C |
| ATOM | 5056 | C | THR | D | 311 | 16.823 | 45.213 | 33.101 | 1.00 | 50.00 | C |
| ATOM | 5057 | O | THR | D | 311 | 15.968 | 44.788 | 32.321 | 1.00 | 50.00 | O |
| ATOM | 5058 | CB | THR | D | 311 | 17.972 | 47.039 | 31.677 | 1.00 | 50.00 | C |
| ATOM | 5059 | OG1 | THR | D | 311 | 16.823 | 47.045 | 30.842 | 1.00 | 50.00 | O |
| ATOM | 5060 | CG2 | THR | D | 311 | 19.154 | 47.420 | 30.787 | 1.00 | 50.00 | C |
| ATOM | 5061 | N | PRO | D | 312 | 16.537 | 45.200 | 34.396 | 1.00 | 50.00 | N |
| ATOM | 5062 | CA | PRO | D | 312 | 15.195 | 44.890 | 34.893 | 1.00 | 50.00 | C |
| ATOM | 5063 | C | PRO | D | 312 | 14.183 | 46.007 | 34.662 | 1.00 | 50.00 | C |
| ATOM | 5064 | O | PRO | D | 312 | 13.592 | 46.588 | 35.571 | 1.00 | 50.00 | O |
| ATOM | 5065 | CB | PRO | D | 312 | 15.453 | 44.462 | 36.335 | 1.00 | 50.00 | C |
| ATOM | 5066 | CG | PRO | D | 312 | 16.712 | 45.213 | 36.759 | 1.00 | 50.00 | C |
| ATOM | 5067 | CD | PRO | D | 312 | 17.467 | 45.538 | 35.472 | 1.00 | 50.00 | C |
| ATOM | 5068 | N | CYS | D | 313 | 14.061 | 46.354 | 33.391 | 1.00 | 50.00 | N |
| ATOM | 5069 | CA | CYS | D | 313 | 12.798 | 46.552 | 32.687 | 1.00 | 50.00 | C |
| ATOM | 5070 | C | CYS | D | 313 | 12.804 | 46.400 | 31.170 | 1.00 | 50.00 | C |
| ATOM | 5071 | O | CYS | D | 313 | 13.877 | 46.344 | 30.571 | 1.00 | 50.00 | O |
| ATOM | 5072 | CB | CYS | D | 313 | 12.324 | 47.946 | 33.074 | 1.00 | 50.00 | C |
| ATOM | 5073 | SG | CYS | D | 313 | 13.682 | 48.980 | 33.653 | 1.00 | 50.00 | S |
| ATOM | 5074 | N | TRP | D | 314 | 11.741 | 46.589 | 30.402 | 1.00 | 50.00 | N |
| ATOM | 5075 | CA | TRP | D | 314 | 10.363 | 46.115 | 30.550 | 1.00 | 50.00 | C |
| ATOM | 5076 | C | TRP | D | 314 | 9.500 | 47.046 | 29.698 | 1.00 | 50.00 | C |
| ATOM | 5077 | O | TRP | D | 314 | 9.441 | 48.235 | 30.006 | 1.00 | 50.00 | O |
| ATOM | 5078 | CB | TRP | D | 314 | 9.722 | 45.731 | 31.891 | 1.00 | 50.00 | C |
| ATOM | 5079 | CG | TRP | D | 314 | 10.180 | 44.578 | 32.765 | 1.00 | 50.00 | C |
| ATOM | 5080 | CD1 | TRP | D | 314 | 11.069 | 43.637 | 32.256 | 1.00 | 50.00 | C |
| ATOM | 5081 | CD2 | TRP | D | 314 | 10.323 | 44.424 | 34.145 | 1.00 | 50.00 | C |
| ATOM | 5082 | NE1 | TRP | D | 314 | 11.658 | 42.828 | 33.206 | 1.00 | 50.00 | N |
| ATOM | 5083 | CE2 | TRP | D | 314 | 11.190 | 43.351 | 34.389 | 1.00 | 50.00 | C |
| ATOM | 5084 | CE3 | TRP | D | 314 | 10.119 | 45.314 | 35.145 | 1.00 | 50.00 | C |
| ATOM | 5085 | CZ2 | TRP | D | 314 | 11.829 | 43.212 | 35.571 | 1.00 | 50.00 | C |
| ATOM | 5086 | CZ3 | TRP | D | 314 | 10.755 | 45.157 | 36.325 | 1.00 | 50.00 | C |
| ATOM | 5087 | CH2 | TRP | D | 314 | 11.598 | 44.125 | 36.534 | 1.00 | 50.00 | C |
| ATOM | 5088 | N | LYS | D | 315 | 8.821 | 46.579 | 28.658 | 1.00 | 50.00 | N |
| ATOM | 5089 | CA | LYS | D | 315 | 7.726 | 47.231 | 27.933 | 1.00 | 50.00 | C |
| ATOM | 5090 | C | LYS | D | 315 | 6.359 | 47.045 | 28.581 | 1.00 | 50.00 | C |
| ATOM | 5091 | O | LYS | D | 315 | 5.999 | 45.921 | 28.927 | 1.00 | 50.00 | O |
| ATOM | 5092 | CB | LYS | D | 315 | 7.543 | 46.681 | 26.517 | 1.00 | 50.00 | C |
| ATOM | 5093 | CG | LYS | D | 315 | 8.672 | 47.029 | 25.553 | 1.00 | 50.00 | C |
| ATOM | 5094 | CD | LYS | D | 315 | 8.535 | 46.504 | 24.126 | 1.00 | 50.00 | C |
| ATOM | 5095 | CE | LYS | D | 315 | 9.769 | 46.898 | 23.319 | 1.00 | 50.00 | C |
| ATOM | 5096 | NZ | LYS | D | 315 | 9.658 | 46.559 | 21.894 | 1.00 | 50.00 | N |
| ATOM | 5097 | N | LEU | D | 316 | 5.587 | 48.105 | 28.758 | 1.00 | 50.00 | N |
| ATOM | 5098 | CA | LEU | D | 316 | 4.402 | 48.108 | 29.617 | 1.00 | 50.00 | C |
| ATOM | 5099 | C | LEU | D | 316 | 3.228 | 48.684 | 28.832 | 1.00 | 50.00 | C |
| ATOM | 5100 | O | LEU | D | 316 | 3.149 | 49.893 | 28.618 | 1.00 | 50.00 | O |
| ATOM | 5101 | CB | LEU | D | 316 | 4.677 | 48.818 | 30.946 | 1.00 | 50.00 | C |
| ATOM | 5102 | CG | LEU | D | 316 | 5.519 | 48.105 | 32.007 | 1.00 | 50.00 | C |
| ATOM | 5103 | CD1 | LEU | D | 316 | 7.020 | 48.064 | 31.731 | 1.00 | 50.00 | C |
| ATOM | 5104 | CD2 | LEU | D | 316 | 5.410 | 48.711 | 33.406 | 1.00 | 50.00 | C |
| ATOM | 5105 | N | HIS | D | 317 | 2.296 | 47.837 | 28.424 | 1.00 | 50.00 | N |
| ATOM | 5106 | CA | HIS | D | 317 | 1.081 | 48.194 | 27.690 | 1.00 | 50.00 | C |
| ATOM | 5107 | C | HIS | D | 317 | 0.064 | 48.909 | 28.573 | 1.00 | 50.00 | C |
| ATOM | 5108 | O | HIS | D | 317 | −0.290 | 48.442 | 29.655 | 1.00 | 50.00 | O |
| ATOM | 5109 | CB | HIS | D | 317 | 0.469 | 46.955 | 27.033 | 1.00 | 50.00 | C |
| ATOM | 5110 | CG | HIS | D | 317 | −0.571 | 47.192 | 25.959 | 1.00 | 50.00 | C |
| ATOM | 5111 | ND1 | HIS | D | 317 | −0.303 | 47.113 | 24.606 | 1.00 | 50.00 | N |
| ATOM | 5112 | CD2 | HIS | D | 317 | −1.908 | 47.521 | 26.167 | 1.00 | 50.00 | C |
| ATOM | 5113 | CE1 | HIS | D | 317 | −1.550 | 47.424 | 24.105 | 1.00 | 50.00 | C |
| ATOM | 5114 | NE2 | HIS | D | 317 | −2.595 | 47.639 | 24.977 | 1.00 | 50.00 | N |
| ATOM | 5115 | N | THR | D | 318 | −0.425 | 50.025 | 28.057 | 1.00 | 50.00 | N |
| ATOM | 5116 | CA | THR | D | 318 | −1.460 | 50.863 | 28.663 | 1.00 | 50.00 | C |
| ATOM | 5117 | C | THR | D | 318 | −2.695 | 50.851 | 27.769 | 1.00 | 50.00 | C |
| ATOM | 5118 | O | THR | D | 318 | −2.615 | 51.038 | 26.555 | 1.00 | 50.00 | O |
| ATOM | 5119 | CB | THR | D | 318 | −0.869 | 52.260 | 28.858 | 1.00 | 50.00 | C |
| ATOM | 5120 | OG1 | THR | D | 318 | 0.147 | 52.225 | 29.851 | 1.00 | 50.00 | O |
| ATOM | 5121 | CG2 | THR | D | 318 | −1.837 | 53.371 | 29.263 | 1.00 | 50.00 | C |
| ATOM | 5122 | N | SER | D | 319 | −3.852 | 50.723 | 28.398 | 1.00 | 50.00 | N |
| ATOM | 5123 | CA | SER | D | 319 | −5.158 | 51.040 | 27.818 | 1.00 | 50.00 | C |
| ATOM | 5124 | C | SER | D | 319 | −5.926 | 52.000 | 28.722 | 1.00 | 50.00 | C |
| ATOM | 5125 | O | SER | D | 319 | −6.012 | 51.693 | 29.911 | 1.00 | 50.00 | O |
| ATOM | 5126 | CB | SER | D | 319 | −6.010 | 49.785 | 27.634 | 1.00 | 50.00 | C |
| ATOM | 5127 | OG | SER | D | 319 | −5.465 | 48.929 | 26.640 | 1.00 | 50.00 | O |
| ATOM | 5128 | N | PRO | D | 320 | −6.557 | 53.080 | 28.279 | 1.00 | 50.00 | N |
| ATOM | 5129 | CA | PRO | D | 320 | −7.401 | 53.923 | 29.126 | 1.00 | 50.00 | C |
| ATOM | 5130 | C | PRO | D | 320 | −8.901 | 53.701 | 28.973 | 1.00 | 50.00 | C |
| ATOM | 5131 | O | PRO | D | 320 | −9.450 | 53.779 | 27.874 | 1.00 | 50.00 | O |
| ATOM | 5132 | CB | PRO | D | 320 | −6.953 | 55.329 | 28.732 | 1.00 | 50.00 | C |
| ATOM | 5133 | CG | PRO | D | 320 | −6.592 | 55.221 | 27.253 | 1.00 | 50.00 | C |
| ATOM | 5134 | CD | PRO | D | 320 | −6.232 | 53.755 | 27.024 | 1.00 | 50.00 | C |
| ATOM | 5135 | N | LEU | D | 321 | −9.563 | 53.512 | 30.103 | 1.00 | 50.00 | N |
| ATOM | 5136 | CA | LEU | D | 321 | −11.019 | 53.598 | 30.210 | 1.00 | 50.00 | C |
| ATOM | 5137 | C | LEU | D | 321 | −11.482 | 55.043 | 30.342 | 1.00 | 50.00 | C |
| ATOM | 5138 | O | LEU | D | 321 | −10.800 | 55.964 | 30.791 | 1.00 | 50.00 | O |
| ATOM | 5139 | CB | LEU | D | 321 | −11.560 | 52.802 | 31.399 | 1.00 | 50.00 | C |
| ATOM | 5140 | CG | LEU | D | 321 | −11.785 | 51.301 | 31.222 | 1.00 | 50.00 | C |
| ATOM | 5141 | CD1 | LEU | D | 321 | −12.858 | 51.004 | 30.177 | 1.00 | 50.00 | C |
| ATOM | 5142 | CD2 | LEU | D | 321 | −10.497 | 50.530 | 30.944 | 1.00 | 50.00 | C |
| ATOM | 5143 | N | CYS | D | 322 | −12.754 | 55.153 | 30.003 | 1.00 | 50.00 | N |
| ATOM | 5144 | CA | CYS | D | 322 | −13.573 | 56.339 | 30.232 | 1.00 | 50.00 | C |
| ATOM | 5145 | C | CYS | D | 322 | −14.222 | 56.390 | 31.613 | 1.00 | 50.00 | C |
| ATOM | 5146 | O | CYS | D | 322 | −15.355 | 55.944 | 31.793 | 1.00 | 50.00 | O |
| ATOM | 5147 | CB | CYS | D | 322 | −14.418 | 56.285 | 28.959 | 1.00 | 50.00 | C |
| ATOM | 5148 | SG | CYS | D | 322 | −13.449 | 56.973 | 27.602 | 1.00 | 50.00 | S |
| ATOM | 5149 | N | THR | D | 323 | −13.556 | 57.044 | 32.554 | 1.00 | 50.00 | N |
| ATOM | 5150 | CA | THR | D | 323 | −14.072 | 57.532 | 33.838 | 1.00 | 50.00 | C |
| ATOM | 5151 | C | THR | D | 323 | −14.702 | 58.921 | 33.898 | 1.00 | 50.00 | C |
| ATOM | 5152 | O | THR | D | 323 | −14.180 | 59.868 | 33.315 | 1.00 | 50.00 | O |
| ATOM | 5153 | CB | THR | D | 323 | −12.866 | 57.591 | 34.779 | 1.00 | 50.00 | C |
| ATOM | 5154 | OG1 | THR | D | 323 | −12.467 | 56.256 | 35.051 | 1.00 | 50.00 | O |
| ATOM | 5155 | CG2 | THR | D | 323 | −12.974 | 58.324 | 36.119 | 1.00 | 50.00 | C |
| ATOM | 5156 | N | THR | D | 324 | −15.579 | 59.247 | 34.834 | 1.00 | 50.00 | N |
| ATOM | 5157 | CA | THR | D | 324 | −15.918 | 60.636 | 35.154 | 1.00 | 50.00 | C |
| ATOM | 5158 | C | THR | D | 324 | −14.788 | 61.486 | 35.732 | 1.00 | 50.00 | C |
| ATOM | 5159 | O | THR | D | 324 | −13.987 | 61.983 | 34.941 | 1.00 | 50.00 | O |
| ATOM | 5160 | CB | THR | D | 324 | −17.312 | 60.669 | 35.782 | 1.00 | 50.00 | C |
| ATOM | 5161 | OG1 | THR | D | 324 | −18.254 | 59.995 | 34.958 | 1.00 | 50.00 | O |
| ATOM | 5162 | CG2 | THR | D | 324 | −17.878 | 62.072 | 35.988 | 1.00 | 50.00 | C |
| ATOM | 5163 | N | ASN | D | 325 | −14.675 | 61.710 | 37.032 | 1.00 | 50.00 | N |
| ATOM | 5164 | CA | ASN | D | 325 | −13.720 | 62.632 | 37.651 | 1.00 | 50.00 | C |
| ATOM | 5165 | C | ASN | D | 325 | −13.800 | 64.065 | 37.129 | 1.00 | 50.00 | C |
| ATOM | 5166 | O | ASN | D | 325 | −14.696 | 64.805 | 37.532 | 1.00 | 50.00 | O |
| ATOM | 5167 | CB | ASN | D | 325 | −12.315 | 62.044 | 37.813 | 1.00 | 50.00 | C |
| ATOM | 5168 | CG | ASN | D | 325 | −12.055 | 60.822 | 38.684 | 1.00 | 50.00 | C |
| ATOM | 5169 | OD1 | ASN | D | 325 | −11.162 | 60.029 | 38.392 | 1.00 | 50.00 | O |
| ATOM | 5170 | ND2 | ASN | D | 325 | −12.748 | 60.638 | 39.794 | 1.00 | 50.00 | N |
| ATOM | 5171 | N | THR | D | 326 | −12.927 | 64.445 | 36.209 | 1.00 | 50.00 | N |
| ATOM | 5172 | CA | THR | D | 326 | −13.080 | 65.600 | 35.323 | 1.00 | 50.00 | C |
| ATOM | 5173 | C | THR | D | 326 | −13.466 | 65.061 | 33.949 | 1.00 | 50.00 | C |
| ATOM | 5174 | O | THR | D | 326 | −12.741 | 64.258 | 33.363 | 1.00 | 50.00 | O |
| ATOM | 5175 | CB | THR | D | 326 | −11.782 | 66.410 | 35.332 | 1.00 | 50.00 | C |
| ATOM | 5176 | OG1 | THR | D | 326 | −11.573 | 66.962 | 36.625 | 1.00 | 50.00 | O |
| ATOM | 5177 | CG2 | THR | D | 326 | −11.718 | 67.576 | 34.348 | 1.00 | 50.00 | C |
| ATOM | 5178 | N | LYS | D | 327 | −14.608 | 65.491 | 33.436 | 1.00 | 50.00 | N |
| ATOM | 5179 | CA | LYS | D | 327 | −15.218 | 64.973 | 32.210 | 1.00 | 50.00 | C |
| ATOM | 5180 | C | LYS | D | 327 | −15.908 | 66.091 | 31.433 | 1.00 | 50.00 | C |
| ATOM | 5181 | O | LYS | D | 327 | −16.310 | 67.104 | 32.005 | 1.00 | 50.00 | O |
| ATOM | 5182 | CB | LYS | D | 327 | −16.194 | 63.871 | 32.626 | 1.00 | 50.00 | C |
| ATOM | 5183 | CG | LYS | D | 327 | −16.641 | 62.904 | 31.531 | 1.00 | 50.00 | C |
| ATOM | 5184 | CD | LYS | D | 327 | −17.733 | 61.903 | 31.904 | 1.00 | 50.00 | C |
| ATOM | 5185 | CE | LYS | D | 327 | −17.959 | 60.923 | 30.757 | 1.00 | 50.00 | C |
| ATOM | 5186 | NZ | LYS | D | 327 | −19.076 | 59.972 | 30.835 | 1.00 | 50.00 | N |
| ATOM | 5187 | N | GLU | D | 328 | −16.103 | 65.915 | 30.136 | 1.00 | 50.00 | N |
| ATOM | 5188 | CA | GLU | D | 328 | −16.988 | 66.749 | 29.319 | 1.00 | 50.00 | C |
| ATOM | 5189 | C | GLU | D | 328 | −17.883 | 65.814 | 28.512 | 1.00 | 50.00 | C |
| ATOM | 5190 | O | GLU | D | 328 | −17.410 | 65.141 | 27.598 | 1.00 | 50.00 | O |
| ATOM | 5191 | CB | GLU | D | 328 | −16.234 | 67.756 | 28.448 | 1.00 | 50.00 | C |
| ATOM | 5192 | CG | GLU | D | 328 | −15.265 | 68.727 | 29.121 | 1.00 | 50.00 | C |
| ATOM | 5193 | CD | GLU | D | 328 | −5.959 | 69.941 | 29.719 | 1.00 | 50.00 | C |
| ATOM | 5194 | OE1 | GLU | D | 328 | −16.462 | 69.853 | 30.860 | 1.00 | 50.00 | O |
| ATOM | 5195 | OE2 | GLU | D | 328 | −16.020 | 70.990 | 29.043 | 1.00 | 50.00 | O |
| ATOM | 5196 | N | GLY | D | 329 | −19.167 | 65.696 | 28.811 | 1.00 | 50.00 | N |
| ATOM | 5197 | CA | GLY | D | 329 | −20.079 | 64.797 | 28.102 | 1.00 | 50.00 | C |
| ATOM | 5198 | C | GLY | D | 329 | −19.860 | 63.305 | 28.319 | 1.00 | 50.00 | C |
| ATOM | 5199 | O | GLY | D | 329 | −19.851 | 62.880 | 29.474 | 1.00 | 50.00 | O |
| ATOM | 5200 | N | SER | D | 330 | −19.683 | 62.537 | 27.254 | 1.00 | 50.00 | N |
| ATOM | 5201 | CA | SER | D | 330 | −19.099 | 61.194 | 27.296 | 1.00 | 50.00 | C |
| ATOM | 5202 | C | SER | D | 330 | −17.587 | 61.095 | 27.115 | 1.00 | 50.00 | C |
| ATOM | 5203 | O | SER | D | 330 | −17.075 | 59.999 | 26.889 | 1.00 | 50.00 | O |
| ATOM | 5204 | CB | SER | D | 330 | −19.794 | 60.289 | 26.280 | 1.00 | 50.00 | C |
| ATOM | 5205 | OG | SER | D | 330 | −21.160 | 60.090 | 26.611 | 1.00 | 50.00 | O |
| ATOM | 5206 | N | ASN | D | 331 | −16.840 | 62.179 | 27.252 | 1.00 | 50.00 | N |
| ATOM | 5207 | CA | ASN | D | 331 | −15.394 | 62.216 | 27.039 | 1.00 | 50.00 | C |
| ATOM | 5208 | C | ASN | D | 331 | −14.671 | 62.368 | 28.369 | 1.00 | 50.00 | C |
| ATOM | 5209 | O | ASN | D | 331 | −14.928 | 63.278 | 29.155 | 1.00 | 50.00 | O |
| ATOM | 5210 | CB | ASN | D | 331 | −14.994 | 63.281 | 26.020 | 1.00 | 50.00 | C |
| ATOM | 5211 | CG | ASN | D | 331 | −14.988 | 64.786 | 26.244 | 1.00 | 50.00 | C |
| ATOM | 5212 | OD1 | ASN | D | 331 | −15.594 | 65.538 | 25.483 | 1.00 | 50.00 | O |
| ATOM | 5213 | ND2 | ASN | D | 331 | −14.287 | 65.281 | 27.248 | 1.00 | 50.00 | N |
| ATOM | 5214 | N | ILE | D | 332 | −13.720 | 61.477 | 28.588 | 1.00 | 50.00 | N |
| ATOM | 5215 | CA | ILE | D | 332 | −12.987 | 61.384 | 29.849 | 1.00 | 50.00 | C |
| ATOM | 5216 | C | ILE | D | 332 | −11.702 | 62.001 | 29.324 | 1.00 | 50.00 | C |
| ATOM | 5217 | O | ILE | D | 332 | −11.732 | 63.222 | 29.176 | 1.00 | 50.00 | O |
| ATOM | 5218 | CB | ILE | D | 332 | −13.018 | 60.018 | 30.535 | 1.00 | 50.00 | C |
| ATOM | 5219 | CG1 | ILE | D | 332 | −14.491 | 59.618 | 30.663 | 1.00 | 50.00 | C |
| ATOM | 5220 | CG2 | ILE | D | 332 | −12.034 | 59.980 | 31.708 | 1.00 | 50.00 | C |
| ATOM | 5221 | CD1 | ILE | D | 332 | −15.360 | 59.238 | 29.466 | 1.00 | 50.00 | C |
| ATOM | 5222 | N | CYS | D | 333 | −10.578 | 61.408 | 28.964 | 1.00 | 50.00 | N |
| ATOM | 5223 | CA | CYS | D | 333 | −10.227 | 59.989 | 28.891 | 1.00 | 50.00 | C |
| ATOM | 5224 | C | CYS | D | 333 | −8.759 | 60.163 | 28.519 | 1.00 | 50.00 | C |
| ATOM | 5225 | O | CYS | D | 333 | −8.384 | 60.815 | 27.544 | 1.00 | 50.00 | O |
| ATOM | 5226 | CB | CYS | D | 333 | −10.883 | 59.062 | 27.868 | 1.00 | 50.00 | C |
| ATOM | 5227 | SG | CYS | D | 333 | −12.667 | 58.816 | 27.745 | 1.00 | 50.00 | S |
| ATOM | 5228 | N | LEU | D | 334 | −7.894 | 59.584 | 29.333 | 1.00 | 50.00 | N |
| ATOM | 5229 | CA | LEU | D | 334 | −6.540 | 60.101 | 29.526 | 1.00 | 50.00 | C |
| ATOM | 5230 | C | LEU | D | 334 | −5.607 | 59.958 | 28.332 | 1.00 | 50.00 | C |
| ATOM | 5231 | O | LEU | D | 334 | −5.714 | 59.022 | 27.540 | 1.00 | 50.00 | O |
| ATOM | 5232 | CB | LEU | D | 334 | −5.967 | 59.430 | 30.772 | 1.00 | 50.00 | C |
| ATOM | 5233 | CG | LEU | D | 334 | −6.486 | 60.083 | 32.052 | 1.00 | 50.00 | C |
| ATOM | 5234 | CD1 | LEU | D | 334 | −7.94 | 259.790 | 32.415 | 1.00 | 50.00 | C |
| ATOM | 5235 | CD2 | LEU | D | 334 | −5.597 | 59.635 | 33.203 | 1.00 | 50.00 | C |
| ATOM | 5236 | N | THR | D | 335 | −4.709 | 60.925 | 28.232 | 1.00 | 50.00 | N |
| ATOM | 5237 | CA | THR | D | 335 | −3.758 | 61.017 | 27.126 | 1.00 | 50.00 | C |
| ATOM | 5238 | C | THR | D | 335 | −2.452 | 60.406 | 27.615 | 1.00 | 50.00 | C |
| ATOM | 5239 | O | THR | D | 335 | −1.650 | 61.041 | 28.298 | 1.00 | 50.00 | O |
| ATOM | 5240 | CB | THR | D | 335 | −3.585 | 62.449 | 26.619 | 1.00 | 50.00 | C |
| ATOM | 5241 | OG1 | THR | D | 335 | −4.832 | 62.924 | 26.132 | 1.00 | 50.00 | O |
| ATOM | 5242 | CG2 | THR | D | 335 | −2.558 | 62.564 | 25.494 | 1.00 | 50.00 | C |
| ATOM | 5243 | N | ARG | D | 336 | −2.279 | 59.161 | 27.206 | 1.00 | 50.00 | N |
| ATOM | 5244 | CA | ARG | D | 336 | −1.035 | 58.412 | 27.376 | 1.00 | 50.00 | C |
| ATOM | 5245 | C | ARG | D | 336 | −0.905 | 57.489 | 26.172 | 1.00 | 50.00 | C |
| ATOM | 5246 | O | ARG | D | 336 | −1.803 | 57.262 | 25.361 | 1.00 | 50.00 | O |
| ATOM | 5247 | CB | ARG | D | 336 | −0.980 | 7.638 | 28.693 | 1.00 | 50.00 | C |
| ATOM | 5248 | CG | ARG | D | 336 | −1.126 | 58.555 | 29.905 | 1.00 | 50.00 | C |
| ATOM | 5249 | CD | ARG | D | 336 | −0.850 | 57.843 | 31.223 | 1.00 | 50.00 | C |
| ATOM | 5250 | NE | ARG | D | 336 | −1.075 | 58.827 | 32.281 | 1.00 | 50.00 | N |
| ATOM | 5251 | CZ | ARG | D | 336 | −0.783 | 58.617 | 33.553 | 1.00 | 50.00 | C |
| ATOM | 5252 | NH1 | ARG | D | 336 | 0.026 | 57.674 | 33.988 | 1.00 | 50.00 | N |
| ATOM | 5253 | NH2 | ARG | D | 336 | −1.411 | 59.258 | 34.517 | 1.00 | 50.00 | N |
| ATOM | 5254 | N | THR | D | 337 | 0.288 | 56.930 | 26.078 | 1.00 | 50.00 | N |
| ATOM | 5255 | CA | THR | D | 337 | 0.660 | 56.051 | 24.971 | 1.00 | 50.00 | C |
| ATOM | 5256 | C | THR | D | 337 | 0.125 | 54.633 | 25.131 | 1.00 | 50.00 | C |
| ATOM | 5257 | O | THR | D | 337 | −0.252 | 54.202 | 26.220 | 1.00 | 50.00 | O |
| ATOM | 5258 | CB | THR | D | 337 | 2.178 | 56.144 | 24.821 | 1.00 | 50.00 | C |
| ATOM | 5259 | OG1 | THR | D | 337 | 2.891 | 55.651 | 25.947 | 1.00 | 50.00 | O |
| ATOM | 5260 | CG2 | THR | D | 337 | 2.702 | 57.513 | 24.391 | 1.00 | 50.00 | C |
| ATOM | 5261 | N | ASP | D | 338 | 0.094 | 53.905 | 24.027 | 1.00 | 50.00 | N |
| ATOM | 5262 | CA | ASP | D | 338 | −0.284 | 52.492 | 24.000 | 1.00 | 50.00 | C |
| ATOM | 5263 | C | ASP | D | 338 | 0.697 | 51.530 | 24.662 | 1.00 | 50.00 | C |
| ATOM | 5264 | O | ASP | D | 338 | 0.239 | 50.568 | 25.276 | 1.00 | 50.00 | O |
| ATOM | 5265 | CB | ASP | D | 338 | −0.587 | 52.050 | 22.567 | 1.00 | 50.00 | C |
| ATOM | 5266 | CG | ASP | D | 338 | −1.812 | 52.681 | 21.921 | 1.00 | 50.00 | C |
| ATOM | 5267 | OD1 | ASP | D | 338 | −2.929 | 52.532 | 22.464 | 1.00 | 50.00 | O |
| ATOM | 5268 | OD2 | ASP | D | 338 | −1.670 | 53.335 | 20.866 | 1.00 | 50.00 | O |
| ATOM | 5269 | N | ARG | D | 339 | 1.998 | 51.728 | 24.522 | 1.00 | 50.00 | N |
| ATOM | 5270 | CA | ARG | D | 339 | 3.044 | 50.875 | 25.088 | 1.00 | 50.00 | C |
| ATOM | 5271 | C | ARG | D | 339 | 4.150 | 51.785 | 25.612 | 1.00 | 50.00 | C |
| ATOM | 5272 | O | ARG | D | 339 | 4.486 | 52.763 | 24.948 | 1.00 | 50.00 | O |
| ATOM | 5273 | CB | ARG | D | 339 | 3.541 | 49.852 | 24.065 | 1.00 | 50.00 | C |
| ATOM | 5274 | CG | ARG | D | 339 | 2.464 | 48.916 | 23.517 | 1.00 | 50.00 | C |
| ATOM | 5275 | CD | ARG | D | 339 | 2.911 | 47.815 | 22.558 | 1.00 | 50.00 | C |
| ATOM | 5276 | NE | ARG | D | 339 | 1.741 | 47.166 | 21.966 | 1.00 | 50.00 | N |
| ATOM | 5277 | CZ | ARG | D | 339 | 1.771 | 46.233 | 21.029 | 1.00 | 50.00 | C |
| ATOM | 5278 | NH1 | ARG | D | 339 | 2.880 | 45.707 | 20.543 | 1.00 | 50.00 | N |
| ATOM | 5279 | NH2 | ARG | D | 339 | 0.623 | 45.797 | 20.545 | 1.00 | 50.00 | N |
| ATOM | 5280 | N | GLY | D | 340 | 4.720 | 51.507 | 26.774 | 1.00 | 50.00 | N |
| ATOM | 5281 | CA | GLY | D | 340 | 5.743 | 52.328 | 27.423 | 1.00 | 50.00 | C |
| ATOM | 5282 | C | GLY | D | 340 | 6.886 | 51.493 | 27.984 | 1.00 | 50.00 | C |
| ATOM | 5283 | O | GLY | D | 340 | 6.702 | 50.849 | 29.016 | 1.00 | 50.00 | O |
| ATOM | 5284 | N | TRP | D | 341 | 8.059 | 51.526 | 27.372 | 1.00 | 50.00 | N |
| ATOM | 5285 | CA | TRP | D | 341 | 9.290 | 50.913 | 27.873 | 1.00 | 50.00 | C |
| ATOM | 5286 | C | TRP | D | 341 | 9.930 | 51.864 | 28.876 | 1.00 | 50.00 | C |
| ATOM | 5287 | O | TRP | D | 341 | 9.950 | 53.078 | 28.674 | 1.00 | 50.00 | O |
| ATOM | 5288 | CB | TRP | D | 341 | 10.273 | 50.545 | 26.758 | 1.00 | 50.00 | C |
| ATOM | 5289 | CG | TRP | D | 341 | 11.512 | 49.757 | 27.131 | 1.00 | 50.00 | C |
| ATOM | 5290 | CD1 | TRP | D | 341 | 11.574 | 48.382 | 27.340 | 1.00 | 50.00 | C |
| ATOM | 5291 | CD2 | TRP | D | 341 | 12.812 | 50.195 | 27.364 | 1.00 | 50.00 | C |
| ATOM | 5292 | NE1 | TRP | D | 341 | 12.830 | 47.938 | 27.709 | 1.00 | 50.00 | N |
| ATOM | 5293 | CE2 | TRP | D | 341 | 13.587 | 49.091 | 27.728 | 1.00 | 50.00 | C |
| ATOM | 5294 | CE3 | TRP | D | 341 | 13.381 | 51.420 | 27.296 | 1.00 | 50.00 | C |
| ATOM | 5295 | CZ2 | TRP | D | 341 | 14.898 | 49.249 | 28.023 | 1.00 | 50.00 | C |
| ATOM | 5296 | CZ3 | TRP | D | 341 | 14.695 | 51.547 | 27.575 | 1.00 | 50.00 | C |
| ATOM | 5297 | CH2 | TRP | D | 341 | 15.441 | 50.479 | 27.927 | 1.00 | 50.00 | C |
| ATOM | 5298 | N | TYR | D | 342 | 10.484 | 51.294 | 29.933 | 1.00 | 50.00 | N |
| ATOM | 5299 | CA | TYR | D | 342 | 11.270 | 52.049 | 30.908 | 1.00 | 50.00 | C |
| ATOM | 5300 | C | TYR | D | 342 | 12.626 | 51.385 | 31.123 | 1.00 | 50.00 | C |
| ATOM | 5301 | O | TYR | D | 342 | 12.739 | 50.168 | 30.985 | 1.00 | 50.00 | O |
| ATOM | 5302 | CB | TYR | D | 342 | 10.387 | 52.505 | 32.076 | 1.00 | 50.00 | C |
| ATOM | 5303 | CG | TYR | D | 342 | 10.427 | 51.955 | 33.487 | 1.00 | 50.00 | C |
| ATOM | 5304 | CD1 | TYR | D | 342 | 10.826 | 50.684 | 33.688 | 1.00 | 50.00 | C |
| ATOM | 5305 | CD2 | TYR | D | 342 | 10.177 | 52.723 | 34.568 | 1.00 | 50.00 | C |
| ATOM | 5306 | CE1 | TYR | D | 342 | 11.196 | 50.269 | 34.916 | 1.00 | 50.00 | C |
| ATOM | 5307 | CE2 | TYR | D | 342 | 10.431 | 52.272 | 35.815 | 1.00 | 50.00 | C |
| ATOM | 5308 | CZ | TYR | D | 342 | 10.976 | 51.051 | 35.989 | 1.00 | 50.00 | C |
| ATOM | 5309 | OH | TYR | D | 342 | 11.401 | 50.567 | 37.198 | 1.00 | 50.00 | O |
| ATOM | 5310 | N | CYS | D | 343 | 13.665 | 52.115 | 31.501 | 1.00 | 50.00 | N |
| ATOM | 5311 | CA | CYS | D | 343 | 14.735 | 51.549 | 32.323 | 1.00 | 50.00 | C |
| ATOM | 5312 | C | CYS | D | 343 | 15.474 | 52.410 | 33.340 | 1.00 | 50.00 | C |
| ATOM | 5313 | O | CYS | D | 343 | 15.487 | 53.639 | 33.280 | 1.00 | 50.00 | O |
| ATOM | 5314 | CB | CYS | D | 343 | 15.594 | 50.487 | 31.636 | 1.00 | 50.00 | C |
| ATOM | 5315 | SG | CYS | D | 343 | 15.317 | 48.929 | 32.497 | 1.00 | 50.00 | S |
| ATOM | 5316 | N | ASP | D | 344 | 16.031 | 51.722 | 34.325 | 1.00 | 50.00 | N |
| ATOM | 5317 | CA | ASP | D | 344 | 16.475 | 52.290 | 35.596 | 1.00 | 50.00 | C |
| ATOM | 5318 | C | ASP | D | 344 | 17.960 | 52.611 | 35.482 | 1.00 | 50.00 | C |
| ATOM | 5319 | O | ASP | D | 344 | 18.830 | 51.769 | 35.703 | 1.00 | 50.00 | O |
| ATOM | 5320 | CB | ASP | D | 344 | 16.191 | 51.317 | 36.742 | 1.00 | 50.00 | C |
| ATOM | 5321 | CG | ASP | D | 344 | 14.727 | 51.030 | 37.043 | 1.00 | 50.00 | C |
| ATOM | 5322 | OD1 | ASP | D | 344 | 13.972 | 51.980 | 37.342 | 1.00 | 50.00 | O |
| ATOM | 5323 | OD2 | ASP | D | 344 | 14.320 | 49.849 | 36.983 | 1.00 | 50.00 | O |
| ATOM | 5324 | N | ASN | D | 345 | 18.224 | 53.852 | 35.111 | 1.00 | 50.00 | N |
| ATOM | 5325 | CA | ASN | D | 345 | 19.573 | 54.412 | 35.016 | 1.00 | 50.00 | C |
| ATOM | 5326 | C | ASN | D | 345 | 19.980 | 55.128 | 36.300 | 1.00 | 50.00 | C |
| ATOM | 5327 | O | ASN | D | 345 | 21.084 | 54.895 | 36.791 | 1.00 | 50.00 | O |
| ATOM | 5328 | CB | ASN | D | 345 | 19.697 | 55.257 | 33.746 | 1.00 | 50.00 | C |
| ATOM | 5329 | CG | ASN | D | 345 | 19.515 | 54.498 | 32.438 | 1.00 | 50.00 | C |
| ATOM | 5330 | OD1 | ASN | D | 345 | 20.340 | 53.667 | 32.061 | 1.00 | 50.00 | O |
| ATOM | 5331 | ND2 | ASN | D | 345 | 18.422 | 54.714 | 31.728 | 1.00 | 50.00 | N |
| ATOM | 5332 | N | ALA | D | 346 | 19.118 | 55.956 | 36.867 | 1.00 | 50.00 | N |
| ATOM | 5333 | CA | ALA | D | 346 | 19.186 | 56.426 | 38.251 | 1.00 | 50.00 | C |
| ATOM | 5334 | C | ALA | D | 346 | 17.764 | 56.733 | 38.708 | 1.00 | 50.00 | C |
| ATOM | 5335 | O | ALA | D | 346 | 17.226 | 55.995 | 39.533 | 1.00 | 50.00 | O |
| ATOM | 5336 | CB | ALA | D | 346 | 20.177 | 57.576 | 38.422 | 1.00 | 50.00 | C |
| ATOM | 5337 | N | GLY | D | 347 | 17.106 | 57.718 | 38.118 | 1.00 | 50.00 | N |
| ATOM | 5338 | CA | GLY | D | 347 | 15.657 | 57.685 | 37.920 | 1.00 | 50.00 | C |
| ATOM | 5339 | C | GLY | D | 347 | 15.169 | 56.676 | 36.888 | 1.00 | 50.00 | C |
| ATOM | 5340 | O | GLY | D | 347 | 15.921 | 55.895 | 36.306 | 1.00 | 50.00 | O |
| ATOM | 5341 | N | SER | D | 348 | 13.865 | 56.712 | 36.674 | 1.00 | 50.00 | N |
| ATOM | 5342 | CA | SER | D | 348 | 13.171 | 55.818 | 35.747 | 1.00 | 50.00 | C |
| ATOM | 5343 | C | SER | D | 348 | 12.939 | 56.540 | 34.425 | 1.00 | 50.00 | C |
| ATOM | 5344 | O | SER | D | 348 | 12.243 | 57.552 | 34.360 | 1.00 | 50.00 | O |
| ATOM | 5345 | CB | SER | D | 348 | 11.864 | 55.389 | 36.409 | 1.00 | 50.00 | C |
| ATOM | 5346 | OG | SER | D | 348 | 12.121 | 54.489 | 37.478 | 1.00 | 50.00 | O |
| ATOM | 5347 | N | VAL | D | 349 | 13.551 | 56.015 | 33.377 | 1.00 | 50.00 | N |
| ATOM | 5348 | CA | VAL | D | 349 | 13.608 | 56.640 | 32.055 | 1.00 | 50.00 | C |
| ATOM | 5349 | C | VAL | D | 349 | 12.512 | 55.985 | 31.224 | 1.00 | 50.00 | C |
| ATOM | 5350 | O | VAL | D | 349 | 12.631 | 54.793 | 30.947 | 1.00 | 50.00 | O |
| ATOM | 5351 | CB | VAL | D | 349 | 15.034 | 56.501 | 31.514 | 1.00 | 50.00 | C |
| ATOM | 5352 | CG1 | VAL | D | 349 | 15.158 | 56.736 | 30.010 | 1.00 | 50.00 | C |
| ATOM | 5353 | CG2 | VAL | D | 349 | 15.997 | 57.467 | 32.203 | 1.00 | 50.00 | C |
| ATOM | 5354 | N | SER | D | 350 | 11.478 | 56.710 | 30.832 | 1.00 | 50.00 | N |
| ATOM | 5355 | CA | SER | D | 350 | 10.180 | 56.135 | 30.477 | 1.00 | 50.00 | C |
| ATOM | 5356 | C | SER | D | 350 | 9.712 | 56.672 | 29.130 | 1.00 | 50.00 | C |
| ATOM | 5357 | O | SER | D | 350 | 9.008 | 57.679 | 29.056 | 1.00 | 50.00 | O |
| ATOM | 5358 | CB | SER | D | 350 | 9.205 | 56.417 | 31.620 | 1.00 | 50.00 | C |
| ATOM | 5359 | OG | SER | D | 350 | 9.573 | 55.726 | 32.805 | 1.00 | 50.00 | O |
| ATOM | 5360 | N | PHE | D | 351 | 10.093 | 56.012 | 28.049 | 1.00 | 50.00 | N |
| ATOM | 5361 | CA | PHE | D | 351 | 9.855 | 56.469 | 26.678 | 1.00 | 50.00 | C |
| ATOM | 5362 | C | PHE | D | 351 | 8.805 | 55.645 | 25.939 | 1.00 | 50.00 | C |
| ATOM | 5363 | O | PHE | D | 351 | 8.418 | 54.580 | 26.418 | 1.00 | 50.00 | O |
| ATOM | 5364 | CB | PHE | D | 351 | 11.220 | 56.546 | 25.989 | 1.00 | 50.00 | C |
| ATOM | 5365 | CG | PHE | D | 351 | 12.167 | 57.633 | 26.455 | 1.00 | 50.00 | C |
| ATOM | 5366 | CD1 | PHE | D | 351 | 11.747 | 58.912 | 26.366 | 1.00 | 50.00 | C |
| ATOM | 5367 | CD2 | PHE | D | 351 | 13.395 | 57.383 | 26.954 | 1.00 | 50.00 | C |
| ATOM | 5368 | CE1 | PHE | D | 351 | 12.533 | 59.930 | 26.768 | 1.00 | 50.00 | C |
| ATOM | 5369 | CE2 | PHE | D | 351 | 14.201 | 58.400 | 27.325 | 1.00 | 50.00 | C |
| ATOM | 5370 | CZ | PHE | D | 351 | 13.763 | 59.673 | 27.254 | 1.00 | 50.00 | C |
| ATOM | 5371 | N | PHE | D | 352 | 8.312 | 56.113 | 24.801 | 1.00 | 50.00 | N |
| ATOM | 5372 | CA | PHE | D | 352 | 7.337 | 55.412 | 23.963 | 1.00 | 50.00 | C |
| ATOM | 5373 | C | PHE | D | 352 | 8.049 | 54.428 | 23.041 | 1.00 | 50.00 | C |
| ATOM | 5374 | O | PHE | D | 352 | 8.559 | 54.889 | 22.020 | 1.00 | 50.00 | O |
| ATOM | 5375 | CB | PHE | D | 352 | 6.388 | 56.265 | 23.114 | 1.00 | 50.00 | C |
| ATOM | 5376 | CG | PHE | D | 352 | 5.403 | 55.560 | 22.205 | 1.00 | 50.00 | C |
| ATOM | 5377 | CD1 | PHE | D | 352 | 4.453 | 54.805 | 22.794 | 1.00 | 50.00 | C |
| ATOM | 5378 | CD2 | PHE | D | 352 | 5.437 | 55.633 | 20.858 | 1.00 | 50.00 | C |
| ATOM | 5379 | CE1 | PHE | D | 352 | 3.531 | 54.150 | 22.062 | 1.00 | 50.00 | C |
| ATOM | 5380 | CE2 | PHE | D | 352 | 4.515 | 54.983 | 20.118 | 1.00 | 50.00 | C |
| ATOM | 5381 | CZ | PHE | D | 352 | 3.560 | 54.245 | 20.718 | 1.00 | 50.00 | C |
| ATOM | 5382 | N | PRO | D | 353 | 8.007 | 53.117 | 23.221 | 1.00 | 50.00 | N |
| ATOM | 5383 | CA | PRO | D | 353 | 8.446 | 52.120 | 22.247 | 1.00 | 50.00 | C |
| ATOM | 5384 | C | PRO | D | 353 | 7.488 | 52.040 | 21.066 | 1.00 | 50.00 | C |
| ATOM | 5385 | O | PRO | D | 353 | 6.276 | 51.871 | 21.191 | 1.00 | 50.00 | O |
| ATOM | 5386 | CB | PRO | D | 353 | 8.464 | 50.821 | 23.052 | 1.00 | 50.00 | C |
| ATOM | 5387 | CG | PRO | D | 353 | 7.371 | 51.001 | 24.101 | 1.00 | 50.00 | C |
| ATOM | 5388 | CD | PRO | D | 353 | 7.612 | 52.463 | 24.463 | 1.00 | 50.00 | C |
| ATOM | 5389 | N | GLN | D | 354 | 8.110 | 52.115 | 19.903 | 1.00 | 50.00 | N |
| ATOM | 5390 | CA | GLN | D | 354 | 7.510 | 51.749 | 18.621 | 1.00 | 50.00 | C |
| ATOM | 5391 | C | GLN | D | 354 | 7.698 | 50.246 | 18.452 | 1.00 | 50.00 | C |
| ATOM | 5392 | O | GLN | D | 354 | 8.694 | 49.779 | 17.898 | 1.00 | 50.00 | O |
| ATOM | 5393 | CB | GLN | D | 354 | 8.151 | 52.586 | 17.513 | 1.00 | 50.00 | C |
| ATOM | 5394 | CG | GLN | D | 354 | 7.872 | 54.088 | 17.562 | 1.00 | 50.00 | C |
| ATOM | 5395 | CD | GLN | D | 354 | 6.482 | 54.530 | 17.125 | 1.00 | 50.00 | C |
| ATOM | 5396 | OE1 | GLN | D | 354 | 5.604 | 53.737 | 16.787 | 1.00 | 50.00 | O |
| ATOM | 5397 | NE2 | GLN | D | 354 | 6.225 | 55.824 | 17.142 | 1.00 | 50.00 | N |
| ATOM | 5398 | N | ALA | D | 355 | 6.723 | 49.498 | 18.943 | 1.00 | 50.00 | N |
| ATOM | 5399 | CA | ALA | D | 355 | 6.636 | 48.047 | 18.769 | 1.00 | 50.00 | C |
| ATOM | 5400 | C | ALA | D | 355 | 6.430 | 47.477 | 17.370 | 1.00 | 50.00 | C |
| ATOM | 5401 | O | ALA | D | 355 | 6.465 | 46.255 | 17.225 | 1.00 | 50.00 | O |
| ATOM | 5402 | CB | ALA | D | 355 | 5.503 | 47.564 | 19.673 | 1.00 | 50.00 | C |
| ATOM | 5403 | N | GLU | D | 356 | 6.278 | 48.299 | 16.344 | 1.00 | 50.00 | N |
| ATOM | 5404 | CA | GLU | D | 356 | 6.363 | 47.927 | 14.931 | 1.00 | 50.00 | C |
| ATOM | 5405 | C | GLU | D | 356 | 7.517 | 47.034 | 14.487 | 1.00 | 50.00 | C |
| ATOM | 5406 | O | GLU | D | 356 | 7.307 | 46.195 | 13.611 | 1.00 | 50.00 | O |
| ATOM | 5407 | CB | GLU | D | 356 | 6.319 | 49.202 | 14.087 | 1.00 | 50.00 | C |
| ATOM | 5408 | CG | GLU | D | 356 | 5.038 | 50.035 | 14.131 | 1.00 | 50.00 | C |
| ATOM | 5409 | CD | GLU | D | 356 | 3.825 | 49.347 | 13.523 | 1.00 | 50.00 | C |
| ATOM | 5410 | OE1 | GLU | D | 356 | 3.103 | 48.636 | 14.255 | 1.00 | 50.00 | O |
| ATOM | 5411 | OE2 | GLU | D | 356 | 3.588 | 49.502 | 12.305 | 1.00 | 50.00 | O |
| ATOM | 5412 | N | THR | D | 357 | 8.690 | 47.156 | 15.086 | 1.00 | 50.00 | N |
| ATOM | 5413 | CA | THR | D | 357 | 9.817 | 46.247 | 14.874 | 1.00 | 50.00 | C |
| ATOM | 5414 | C | THR | D | 357 | 9.826 | 45.021 | 15.786 | 1.00 | 50.00 | C |
| ATOM | 5415 | O | THR | D | 357 | 10.906 | 44.542 | 16.129 | 1.00 | 50.00 | O |
| ATOM | 5416 | CB | THR | D | 357 | 11.072 | 47.122 | 14.934 | 1.00 | 50.00 | C |
| ATOM | 5417 | OG1 | THR | D | 357 | 10.989 | 48.230 | 14.049 | 1.00 | 50.00 | O |
| ATOM | 5418 | CG2 | THR | D | 357 | 12.360 | 46.407 | 14.534 | 1.00 | 50.00 | C |
| ATOM | 5419 | N | CYS | D | 358 | 8.707 | 44.434 | 16.179 | 1.00 | 50.00 | N |
| ATOM | 5420 | CA | CYS | D | 358 | 8.690 | 43.314 | 17.120 | 1.00 | 50.00 | C |
| ATOM | 5421 | C | CYS | D | 358 | 7.544 | 42.338 | 16.880 | 1.00 | 50.00 | C |
| ATOM | 5422 | O | CYS | D | 358 | 6.379 | 42.733 | 16.850 | 1.00 | 50.00 | O |
| ATOM | 5423 | CB | CYS | D | 358 | 8.683 | 43.822 | 18.562 | 1.00 | 50.00 | C |
| ATOM | 5424 | SG | CYS | D | 358 | 9.822 | 45.173 | 18.916 | 1.00 | 50.00 | S |
| ATOM | 5425 | N | LYS | D | 359 | 7.856 | 41.053 | 16.806 | 1.00 | 50.00 | N |
| ATOM | 5426 | CA | LYS | D | 359 | 6.935 | 39.949 | 17.083 | 1.00 | 50.00 | C |
| ATOM | 5427 | C | LYS | D | 359 | 6.772 | 39.846 | 18.596 | 1.00 | 50.00 | C |
| ATOM | 5428 | O | LYS | D | 359 | 7.748 | 39.795 | 19.343 | 1.00 | 50.00 | O |
| ATOM | 5429 | CB | LYS | D | 359 | 7.315 | 38.567 | 16.544 | 1.00 | 50.00 | C |
| ATOM | 5430 | CG | LYS | D | 359 | 7.759 | 38.443 | 15.086 | 1.00 | 50.00 | C |
| ATOM | 5431 | CD | LYS | D | 359 | 8.569 | 37.194 | 14.739 | 1.00 | 50.00 | C |
| ATOM | 5432 | CE | LYS | D | 359 | 9.256 | 37.274 | 13.377 | 1.00 | 50.00 | C |
| ATOM | 5433 | NZ | LYS | D | 359 | 10.104 | 36.106 | 13.092 | 1.00 | 50.00 | N |
| ATOM | 5434 | N | VAL | D | 360 | 5.527 | 39.852 | 19.039 | 1.00 | 50.00 | N |
| ATOM | 5435 | CA | VAL | D | 360 | 5.165 | 40.036 | 20.445 | 1.00 | 50.00 | C |
| ATOM | 5436 | C | VAL | D | 360 | 4.500 | 38.737 | 20.884 | 1.00 | 50.00 | C |
| ATOM | 5437 | O | VAL | D | 360 | 3.401 | 38.386 | 20.455 | 1.00 | 50.00 | O |
| ATOM | 5438 | CB | VAL | D | 360 | 4.300 | 41.279 | 20.670 | 1.00 | 50.00 | C |
| ATOM | 5439 | CG1 | VAL | D | 360 | 3.740 | 41.388 | 22.089 | 1.00 | 50.00 | C |
| ATOM | 5440 | CG2 | VAL | D | 360 | 5.014 | 42.570 | 20.270 | 1.00 | 50.00 | C |
| ATOM | 5441 | N | GLN | D | 361 | 5.148 | 38.099 | 21.842 | 1.00 | 50.00 | N |
| ATOM | 5442 | CA | GLN | D | 361 | 4.543 | 37.082 | 22.701 | 1.00 | 50.00 | C |
| ATOM | 5443 | C | GLN | D | 361 | 3.814 | 37.607 | 23.930 | 1.00 | 50.00 | C |
| ATOM | 5444 | O | GLN | D | 361 | 3.915 | 38.772 | 24.316 | 1.00 | 50.00 | O |
| ATOM | 5445 | CB | GLN | D | 361 | 5.659 | 36.111 | 23.089 | 1.00 | 50.00 | C |
| ATOM | 5446 | CG | GLN | D | 361 | 6.103 | 35.169 | 21.977 | 1.00 | 50.00 | C |
| ATOM | 5447 | CD | GLN | D | 361 | 4.931 | 34.426 | 21.357 | 1.00 | 50.00 | C |
| ATOM | 5448 | OE1 | GLN | D | 361 | 4.183 | 33.747 | 22.059 | 1.00 | 50.00 | O |
| ATOM | 5449 | NE2 | GLN | D | 361 | 4.644 | 34.718 | 20.102 | 1.00 | 50.00 | N |
| ATOM | 5450 | N | SER | D | 362 | 3.113 | 36.682 | 24.566 | 1.00 | 50.00 | N |
| ATOM | 5451 | CA | SER | D | 362 | 2.554 | 36.844 | 25.909 | 1.00 | 50.00 | C |
| ATOM | 5452 | C | SER | D | 362 | 3.576 | 37.284 | 26.952 | 1.00 | 50.00 | C |
| ATOM | 5453 | O | SER | D | 362 | 3.465 | 38.394 | 27.471 | 1.00 | 50.00 | O |
| ATOM | 5454 | CB | SER | D | 362 | 1.830 | 35.555 | 26.299 | 1.00 | 50.00 | C |
| ATOM | 5455 | OG | SER | D | 362 | 0.771 | 35.239 | 25.406 | 1.00 | 50.00 | O |
| ATOM | 5456 | N | ASN | D | 363 | 4.593 | 36.470 | 27.183 | 1.00 | 50.00 | N |
| ATOM | 5457 | CA | ASN | D | 363 | 5.785 | 36.811 | 27.962 | 1.00 | 50.00 | C |
| ATOM | 5458 | C | ASN | D | 363 | 7.061 | 36.969 | 27.139 | 1.00 | 50.00 | C |
| ATOM | 5459 | O | ASN | D | 363 | 8.103 | 36.979 | 27.793 | 1.00 | 50.00 | O |
| ATOM | 5460 | CB | ASN | D | 363 | 6.049 | 35.809 | 29.089 | 1.00 | 50.00 | C |
| ATOM | 5461 | CG | ASN | D | 363 | 4.900 | 35.184 | 29.864 | 1.00 | 50.00 | C |
| ATOM | 5462 | OD1 | ASN | D | 363 | 4.260 | 34.259 | 29.366 | 1.00 | 50.00 | O |
| ATOM | 5463 | ND2 | ASN | D | 363 | 4.626 | 35.672 | 31.059 | 1.00 | 50.00 | N |
| ATOM | 5464 | N | ARG | D | 364 | 7.107 | 37.102 | 25.820 | 1.00 | 50.00 | N |
| ATOM | 5465 | CA | ARG | D | 364 | 8.218 | 37.686 | 25.061 | 1.00 | 50.00 | C |
| ATOM | 5466 | C | ARG | D | 364 | 7.857 | 38.843 | 24.129 | 1.00 | 50.00 | C |
| ATOM | 5467 | O | ARG | D | 364 | 6.688 | 39.153 | 23.903 | 1.00 | 50.00 | O |
| ATOM | 5468 | CB | ARG | D | 364 | 9.430 | 36.832 | 24.685 | 1.00 | 50.00 | C |
| ATOM | 5469 | CG | ARG | D | 364 | 10.115 | 36.148 | 25.866 | 1.00 | 50.00 | C |
| ATOM | 5470 | CD | ARG | D | 364 | 11.504 | 35.590 | 25.567 | 1.00 | 50.00 | C |
| ATOM | 5471 | NE | ARG | D | 364 | 12.007 | 34.727 | 26.633 | 1.00 | 50.00 | N |
| ATOM | 5472 | CZ | ARG | D | 364 | 12.910 | 33.781 | 26.451 | 1.00 | 50.00 | C |
| ATOM | 5473 | NH1 | ARG | D | 364 | 13.848 | 33.777 | 25.527 | 1.00 | 50.00 | N |
| ATOM | 5474 | NH2 | ARG | D | 364 | 12.893 | 32.738 | 27.254 | 1.00 | 50.00 | N |
| ATOM | 5475 | N | VAL | D | 365 | 8.838 | 39.606 | 23.675 | 1.00 | 50.00 | N |
| ATOM | 5476 | CA | VAL | D | 365 | 8.776 | 40.616 | 22.617 | 1.00 | 50.00 | C |
| ATOM | 5477 | C | VAL | D | 365 | 10.186 | 40.451 | 22.065 | 1.00 | 50.00 | C |
| ATOM | 5478 | O | VAL | D | 365 | 11.166 | 40.995 | 22.573 | 1.00 | 50.00 | O |
| ATOM | 5479 | CB | VAL | D | 365 | 8.470 | 42.086 | 22.923 | 1.00 | 50.00 | C |
| ATOM | 5480 | CG1 | VAL | D | 365 | 8.755 | 43.134 | 21.851 | 1.00 | 50.00 | C |
| ATOM | 5481 | CG2 | VAL | D | 365 | 7.056 | 42.396 | 23.410 | 1.00 | 50.00 | C |
| ATOM | 5482 | N | PHE | D | 366 | 10.239 | 39.736 | 20.954 | 1.00 | 50.00 | N |
| ATOM | 5483 | CA | PHE | D | 366 | 11.386 | 39.718 | 20.049 | 1.00 | 50.00 | C |
| ATOM | 5484 | C | PHE | D | 366 | 11.464 | 41.065 | 19.342 | 1.00 | 50.00 | C |
| ATOM | 5485 | O | PHE | D | 366 | 10.509 | 41.424 | 18.657 | 1.00 | 50.00 | O |
| ATOM | 5486 | CB | PHE | D | 366 | 11.254 | 38.445 | 19.206 | 1.00 | 50.00 | C |
| ATOM | 5487 | CG | PHE | D | 366 | 11.146 | 38.223 | 17.711 | 1.00 | 50.00 | C |
| ATOM | 5488 | CD1 | PHE | D | 366 | 10.513 | 39.190 | 17.023 | 1.00 | 50.00 | C |
| ATOM | 5489 | CD2 | PHE | D | 366 | 11.922 | 37.363 | 17.017 | 1.00 | 50.00 | C |
| ATOM | 5490 | CE1 | PHE | D | 366 | 10.904 | 39.522 | 15.777 | 1.00 | 50.00 | C |
| ATOM | 5491 | CE2 | PHE | D | 366 | 12.297 | 37.646 | 15.753 | 1.00 | 50.00 | C |
| ATOM | 5492 | CZ | PHE | D | 366 | 11.818 | 38.754 | 15.152 | 1.00 | 50.00 | C |
| ATOM | 5493 | N | CYS | D | 367 | 12.496 | 41.875 | 19.505 | 1.00 | 50.00 | N |
| ATOM | 5494 | CA | CYS | D | 367 | 12.709 | 43.040 | 18.646 | 1.00 | 50.00 | C |
| ATOM | 5495 | C | CYS | D | 367 | 13.923 | 42.834 | 17.749 | 1.00 | 50.00 | C |
| ATOM | 5496 | O | CYS | D | 367 | 14.935 | 42.258 | 18.146 | 1.00 | 50.00 | O |
| ATOM | 5497 | CB | CYS | D | 367 | 12.798 | 44.330 | 19.457 | 1.00 | 50.00 | C |
| ATOM | 5498 | SG | CYS | D | 367 | 11.222 | 44.670 | 20.255 | 1.00 | 50.00 | S |
| ATOM | 5499 | N | ASP | D | 368 | 13.844 | 43.350 | 16.535 | 1.00 | 50.00 | N |
| ATOM | 5500 | CA | ASP | D | 368 | 15.016 | 43.617 | 15.698 | 1.00 | 50.00 | C |
| ATOM | 5501 | C | ASP | D | 368 | 15.757 | 44.915 | 16.012 | 1.00 | 50.00 | C |
| ATOM | 5502 | O | ASP | D | 368 | 16.969 | 44.973 | 15.803 | 1.00 | 50.00 | O |
| ATOM | 5503 | CB | ASP | D | 368 | 14.619 | 43.509 | 14.225 | 1.00 | 50.00 | C |
| ATOM | 5504 | CG | ASP | D | 368 | 14.052 | 42.176 | 13.754 | 1.00 | 50.00 | C |
| ATOM | 5505 | OD1 | ASP | D | 368 | 14.799 | 41.174 | 13.724 | 1.00 | 50.00 | O |
| ATOM | 5506 | OD2 | ASP | D | 368 | 12.847 | 42.115 | 13.425 | 1.00 | 50.00 | O |
| ATOM | 5507 | N | THR | D | 369 | 15.081 | 45.949 | 16.488 | 1.00 | 50.00 | N |
| ATOM | 5508 | CA | THR | D | 369 | 15.593 | 47.278 | 16.829 | 1.00 | 50.00 | C |
| ATOM | 5509 | C | THR | D | 369 | 14.737 | 47.814 | 17.973 | 1.00 | 50.00 | C |
| ATOM | 5510 | O | THR | D | 369 | 13.626 | 47.355 | 18.241 | 1.00 | 50.00 | O |
| ATOM | 5511 | CB | THR | D | 369 | 15.550 | 48.198 | 15.605 | 1.00 | 50.00 | C |
| ATOM | 5512 | OG1 | THR | D | 369 | 16.321 | 47.653 | 14.544 | 1.00 | 50.00 | O |
| ATOM | 5513 | CG2 | THR | D | 369 | 15.973 | 49.660 | 15.739 | 1.00 | 50.00 | C |
| ATOM | 5514 | N | MET | D | 370 | 15.210 | 48.846 | 18.654 | 1.00 | 50.00 | N |
| ATOM | 5515 | CA | MET | D | 370 | 14.466 | 49.513 | 19.723 | 1.00 | 50.00 | C |
| ATOM | 5516 | C | MET | D | 370 | 14.154 | 50.957 | 19.352 | 1.00 | 50.00 | C |
| ATOM | 5517 | O | MET | D | 370 | 14.779 | 51.893 | 19.850 | 1.00 | 50.00 | O |
| ATOM | 5518 | CB | MET | D | 370 | 15.240 | 49.322 | 21.028 | 1.00 | 50.00 | C |
| ATOM | 5519 | CG | MET | D | 370 | 15.185 | 47.890 | 21.554 | 1.00 | 50.00 | C |
| ATOM | 5520 | SD | MET | D | 370 | 13.499 | 47.343 | 21.876 | 1.00 | 50.00 | S |
| ATOM | 5521 | CE | MET | D | 370 | 13.137 | 48.309 | 23.351 | 1.00 | 50.00 | C |
| ATOM | 5522 | N | ASN | D | 371 | 13.152 | 51.134 | 18.504 | 1.00 | 50.00 | N |
| ATOM | 5523 | CA | ASN | D | 371 | 12.684 | 52.441 | 18.040 | 1.00 | 50.00 | C |
| ATOM | 5524 | C | ASN | D | 371 | 11.827 | 53.114 | 19.112 | 1.00 | 50.00 | C |
| ATOM | 5525 | O | ASN | D | 371 | 10.794 | 52.532 | 19.439 | 1.00 | 50.00 | O |
| ATOM | 5526 | CB | ASN | D | 371 | 12.006 | 52.258 | 16.678 | 1.00 | 50.00 | C |
| ATOM | 5527 | CG | ASN | D | 371 | 12.739 | 52.020 | 15.360 | 1.00 | 50.00 | C |
| ATOM | 5528 | OD1 | ASN | D | 371 | 13.270 | 52.936 | 14.734 | 1.00 | 50.00 | O |
| ATOM | 5529 | ND2 | ASN | D | 371 | 12.683 | 50.873 | 14.709 | 1.00 | 50.00 | N |
| ATOM | 5530 | N | SER | D | 372 | 12.190 | 54.233 | 19.724 | 1.00 | 50.00 | N |
| ATOM | 5531 | CA | SER | D | 372 | 11.455 | 54.900 | 20.804 | 1.00 | 50.00 | C |
| ATOM | 5532 | C | SER | D | 372 | 11.455 | 56.428 | 20.841 | 1.00 | 50.00 | C |
| ATOM | 5533 | O | SER | D | 372 | 12.395 | 57.059 | 20.357 | 1.00 | 50.00 | O |
| ATOM | 5534 | CB | SER | D | 372 | 11.981 | 54.372 | 22.139 | 1.00 | 50.00 | C |
| ATOM | 5535 | OG | SER | D | 372 | 11.174 | 54.770 | 23.238 | 1.00 | 50.00 | O |
| ATOM | 5536 | N | LEU | D | 373 | 10.451 | 57.068 | 21.425 | 1.00 | 50.00 | N |
| ATOM | 5537 | CA | LEU | D | 373 | 10.172 | 58.506 | 21.350 | 1.00 | 50.00 | C |
| ATOM | 5538 | C | LEU | D | 373 | 10.077 | 59.187 | 22.714 | 1.00 | 50.00 | C |
| ATOM | 5539 | O | LEU | D | 373 | 9.618 | 58.562 | 23.670 | 1.00 | 50.00 | O |
| ATOM | 5540 | CB | LEU | D | 373 | 8.872 | 58.729 | 20.570 | 1.00 | 50.00 | C |
| ATOM | 5541 | CG | LEU | D | 373 | 8.870 | 58.650 | 19.042 | 1.00 | 50.00 | C |
| ATOM | 5542 | CD1 | LEU | D | 373 | 9.112 | 57.256 | 18.466 | 1.00 | 50.00 | C |
| ATOM | 5543 | CD2 | LEU | D | 373 | 7.549 | 59.137 | 18.447 | 1.00 | 50.00 | C |
| ATOM | 5544 | N | THR | D | 374 | 10.417 | 60.463 | 22.836 | 1.00 | 50.00 | N |
| ATOM | 5545 | CA | THR | D | 374 | 10.185 | 61.298 | 24.020 | 1.00 | 50.00 | C |
| ATOM | 5546 | C | THR | D | 374 | 8.752 | 61.804 | 24.148 | 1.00 | 50.00 | C |
| ATOM | 5547 | O | THR | D | 374 | 8.108 | 62.164 | 23.163 | 1.00 | 50.00 | O |
| ATOM | 5548 | CB | THR | D | 374 | 11.204 | 62.427 | 24.196 | 1.00 | 50.00 | C |
| ATOM | 5549 | OG1 | THR | D | 374 | 12.521 | 61.896 | 24.199 | 1.00 | 50.00 | O |
| ATOM | 5550 | CG2 | THR | D | 374 | 11.085 | 63.275 | 25.463 | 1.00 | 50.00 | C |
| ATOM | 5551 | N | LEU | D | 375 | 8.259 | 61.807 | 25.377 | 1.00 | 50.00 | N |
| ATOM | 5552 | CA | LEU | D | 375 | 6.870 | 62.114 | 25.725 | 1.00 | 50.00 | C |
| ATOM | 5553 | C | LEU | D | 375 | 6.818 | 63.447 | 26.465 | 1.00 | 50.00 | C |
| ATOM | 5554 | O | LEU | D | 375 | 7.779 | 63.759 | 27.169 | 1.00 | 50.00 | O |
| ATOM | 5555 | CB | LEU | D | 375 | 6.201 | 61.005 | 26.543 | 1.00 | 50.00 | C |
| ATOM | 5556 | CG | LEU | D | 375 | 5.823 | 59.729 | 25.789 | 1.00 | 50.00 | C |
| ATOM | 5557 | CD1 | LEU | D | 375 | 7.074 | 59.058 | 25.232 | 1.00 | 50.00 | C |
| ATOM | 5558 | CD2 | LEU | D | 375 | 5.098 | 58.643 | 26.583 | 1.00 | 50.00 | C |
| ATOM | 5559 | N | PRO | D | 376 | 5.744 | 64.224 | 26.427 | 1.00 | 50.00 | N |
| ATOM | 5560 | CA | PRO | D | 376 | 5.518 | 65.359 | 27.324 | 1.00 | 50.00 | C |
| ATOM | 5561 | C | PRO | D | 376 | 5.923 | 65.159 | 28.780 | 1.00 | 50.00 | C |
| ATOM | 5562 | O | PRO | D | 376 | 5.710 | 64.071 | 29.315 | 1.00 | 50.00 | O |
| ATOM | 5563 | CB | PRO | D | 376 | 4.012 | 65.596 | 27.217 | 1.00 | 50.00 | C |
| ATOM | 5564 | CG | PRO | D | 376 | 3.593 | 65.082 | 25.842 | 1.00 | 50.00 | C |
| ATOM | 5565 | CD | PRO | D | 376 | 4.624 | 64.007 | 25.512 | 1.00 | 50.00 | C |
| ATOM | 5566 | N | SER | D | 377 | 6.511 | 66.161 | 29.413 | 1.00 | 50.00 | N |
| ATOM | 5567 | CA | SER | D | 377 | 6.832 | 66.124 | 30.842 | 1.00 | 50.00 | C |
| ATOM | 5568 | C | SER | D | 377 | 5.689 | 65.656 | 31.736 | 1.00 | 50.00 | C |
| ATOM | 5569 | O | SER | D | 377 | 5.858 | 64.672 | 32.453 | 1.00 | 50.00 | O |
| ATOM | 5570 | CB | SER | D | 377 | 7.408 | 67.456 | 31.319 | 1.00 | 50.00 | C |
| ATOM | 5571 | OG | SER | D | 377 | 8.658 | 67.730 | 30.702 | 1.00 | 50.00 | O |
| ATOM | 5572 | N | GLU | D | 378 | 4.511 | 66.238 | 31.578 | 1.00 | 50.00 | N |
| ATOM | 5573 | CA | GLU | D | 378 | 3.244 | 65.790 | 32.155 | 1.00 | 50.00 | C |
| ATOM | 5574 | C | GLU | D | 378 | 2.955 | 64.296 | 32.069 | 1.00 | 50.00 | C |
| ATOM | 5575 | O | GLU | D | 378 | 2.874 | 63.708 | 33.146 | 1.00 | 50.00 | O |
| ATOM | 5576 | CB | GLU | D | 378 | 2.113 | 66.653 | 31.595 | 1.00 | 50.00 | C |
| ATOM | 5577 | CG | GLU | D | 378 | 2.246 | 68.150 | 31.878 | 1.00 | 50.00 | C |
| ATOM | 5578 | CD | GLU | D | 378 | 1.872 | 68.546 | 33.298 | 1.00 | 50.00 | C |
| ATOM | 5579 | OE1 | GLU | D | 378 | 2.775 | 68.613 | 34.160 | 1.00 | 50.00 | O |
| ATOM | 5580 | OE2 | GLU | D | 378 | 0.674 | 68.779 | 33.568 | 1.00 | 50.00 | O |
| ATOM | 5581 | N | VAL | D | 379 | 2.930 | 63.647 | 30.916 | 1.00 | 50.00 | N |
| ATOM | 5582 | CA | VAL | D | 379 | 2.916 | 62.188 | 30.775 | 1.00 | 50.00 | C |
| ATOM | 5583 | C | VAL | D | 379 | 3.890 | 61.425 | 31.669 | 1.00 | 50.00 | C |
| ATOM | 5584 | O | VAL | D | 379 | 3.446 | 60.556 | 32.419 | 1.00 | 50.00 | O |
| ATOM | 5585 | CB | VAL | D | 379 | 3.054 | 61.804 | 29.299 | 1.00 | 50.00 | C |
| ATOM | 5586 | CG1 | VAL | D | 379 | 3.240 | 60.311 | 29.035 | 1.00 | 50.00 | C |
| ATOM | 5587 | CG2 | VAL | D | 379 | 1.911 | 62.259 | 28.390 | 1.00 | 50.00 | C |
| ATOM | 5588 | N | ASN | D | 380 | 5.181 | 61.709 | 31.595 | 1.00 | 50.00 | N |
| ATOM | 5589 | CA | ASN | D | 380 | 6.212 | 61.123 | 32.453 | 1.00 | 50.00 | C |
| ATOM | 5590 | C | ASN | D | 380 | 5.980 | 61.267 | 33.952 | 1.00 | 50.00 | C |
| ATOM | 5591 | O | ASN | D | 380 | 5.905 | 60.252 | 34.642 | 1.00 | 50.00 | O |
| ATOM | 5592 | CB | ASN | D | 380 | 7.599 | 61.669 | 32.106 | 1.00 | 50.00 | C |
| ATOM | 5593 | CG | ASN | D | 380 | 8.207 | 61.296 | 30.762 | 1.00 | 50.00 | C |
| ATOM | 5594 | OD1 | ASN | D | 380 | 8.717 | 62.140 | 30.026 | 1.00 | 50.00 | O |
| ATOM | 5595 | ND2 | ASN | D | 380 | 8.203 | 60.018 | 30.430 | 1.00 | 50.00 | N |
| ATOM | 5596 | N | LEU | D | 381 | 5.800 | 62.485 | 34.435 | 1.00 | 50.00 | N |
| ATOM | 5597 | CA | LEU | D | 381 | 5.236 | 62.823 | 35.743 | 1.00 | 50.00 | C |
| ATOM | 5598 | C | LEU | D | 381 | 4.096 | 61.917 | 36.196 | 1.00 | 50.00 | C |
| ATOM | 5599 | O | LEU | D | 381 | 4.272 | 61.139 | 37.132 | 1.00 | 50.00 | O |
| ATOM | 5600 | CB | LEU | D | 381 | 4.878 | 64.308 | 35.657 | 1.00 | 50.00 | C |
| ATOM | 5601 | CG | LEU | D | 381 | 5.903 | 65.353 | 36.099 | 1.00 | 50.00 | C |
| ATOM | 5602 | CD1 | LEU | D | 381 | 7.177 | 65.368 | 35.256 | 1.00 | 50.00 | C |
| ATOM | 5603 | CD2 | LEU | D | 381 | 5.273 | 66.744 | 36.074 | 1.00 | 50.00 | C |
| ATOM | 5604 | N | CYS | D | 382 | 2.994 | 61.927 | 35.466 | 1.00 | 50.00 | N |
| ATOM | 5605 | CA | CYS | D | 382 | 1.910 | 60.948 | 35.523 | 1.00 | 50.00 | C |
| ATOM | 5606 | C | CYS | D | 382 | 2.226 | 59.459 | 35.639 | 1.00 | 50.00 | C |
| ATOM | 5607 | O | CYS | D | 382 | 1.468 | 58.752 | 36.301 | 1.00 | 50.00 | O |
| ATOM | 5608 | CB | CYS | D | 382 | 1.068 | 61.322 | 34.303 | 1.00 | 50.00 | C |
| ATOM | 5609 | SG | CYS | D | 382 | −0.598 | 61.775 | 34.798 | 1.00 | 50.00 | S |
| ATOM | 5610 | N | ASN | D | 383 | 3.303 | 58.946 | 35.065 | 1.00 | 50.00 | N |
| ATOM | 5611 | CA | ASN | D | 383 | 3.821 | 57.598 | 35.310 | 1.00 | 50.00 | C |
| ATOM | 5612 | C | ASN | D | 383 | 5.167 | 57.559 | 36.029 | 1.00 | 50.00 | C |
| ATOM | 5613 | O | ASN | D | 383 | 6.092 | 56.859 | 35.618 | 1.00 | 50.00 | O |
| ATOM | 5614 | CB | ASN | D | 383 | 3.886 | 56.842 | 33.981 | 1.00 | 50.00 | C |
| ATOM | 5615 | CG | ASN | D | 383 | 2.614 | 56.247 | 33.397 | 1.00 | 50.00 | C |
| ATOM | 5616 | OD1 | ASN | D | 383 | 2.241 | 56.544 | 32.263 | 1.00 | 50.00 | O |
| ATOM | 5617 | ND2 | ASN | D | 383 | 1.906 | 55.428 | 34.154 | 1.00 | 50.00 | N |
| ATOM | 5618 | N | VAL | D | 384 | 5.307 | 58.254 | 37.146 | 1.00 | 50.00 | N |
| ATOM | 5619 | CA | VAL | D | 384 | 6.489 | 58.217 | 38.009 | 1.00 | 50.00 | C |
| ATOM | 5620 | C | VAL | D | 384 | 5.896 | 58.319 | 39.409 | 1.00 | 50.00 | C |
| ATOM | 5621 | O | VAL | D | 384 | 5.261 | 59.319 | 39.745 | 1.00 | 50.00 | O |
| ATOM | 5622 | CB | VAL | D | 384 | 7.532 | 59.293 | 37.690 | 1.00 | 50.00 | C |
| ATOM | 5623 | CG1 | VAL | D | 384 | 8.620 | 59.466 | 38.750 | 1.00 | 50.00 | C |
| ATOM | 5624 | CG2 | VAL | D | 384 | 8.276 | 59.063 | 36.374 | 1.00 | 50.00 | C |
| ATOM | 5625 | N | ASP | D | 385 | 6.124 | 57.310 | 40.235 | 1.00 | 50.00 | N |
| ATOM | 5626 | CA | ASP | D | 385 | 5.652 | 57.252 | 41.620 | 1.00 | 50.00 | C |
| ATOM | 5627 | C | ASP | D | 385 | 4.129 | 57.170 | 41.699 | 1.00 | 50.00 | C |
| ATOM | 5628 | O | ASP | D | 385 | 3.458 | 56.626 | 40.823 | 1.00 | 50.00 | O |
| ATOM | 5629 | CB | ASP | D | 385 | 6.359 | 58.293 | 42.493 | 1.00 | 50.00 | C |
| ATOM | 5630 | CG | ASP | D | 385 | 7.847 | 58.128 | 42.760 | 1.00 | 50.00 | C |
| ATOM | 5631 | OD1 | ASP | D | 385 | 8.240 | 57.110 | 43.370 | 1.00 | 50.00 | O |
| ATOM | 5632 | OD2 | ASP | D | 385 | 8.629 | 59.033 | 42.397 | 1.00 | 50.00 | O |
| ATOM | 5633 | N | ILE | D | 386 | 3.572 | 57.736 | 42.757 | 1.00 | 50.00 | N |
| ATOM | 5634 | CA | ILE | D | 386 | 2.164 | 58.109 | 42.892 | 1.00 | 50.00 | C |
| ATOM | 5635 | C | ILE | D | 386 | 2.113 | 59.610 | 42.606 | 1.00 | 50.00 | C |
| ATOM | 5636 | O | ILE | D | 386 | 1.819 | 60.387 | 43.514 | 1.00 | 50.00 | O |
| ATOM | 5637 | CB | ILE | D | 386 | 1.737 | 57.607 | 44.278 | 1.00 | 50.00 | C |
| ATOM | 5638 | CG1 | ILE | D | 386 | 1.828 | 56.094 | 44.501 | 1.00 | 50.00 | C |
| ATOM | 5639 | CG2 | ILE | D | 386 | 0.335 | 58.024 | 44.720 | 1.00 | 50.00 | C |
| ATOM | 5640 | CD1 | ILE | D | 386 | 1.582 | 55.579 | 45.918 | 1.00 | 50.00 | C |
| ATOM | 5641 | N | PHE | D | 387 | 2.360 | 60.072 | 41.388 | 1.00 | 50.00 | N |
| ATOM | 5642 | CA | PHE | D | 387 | 2.187 | 61.468 | 40.975 | 1.00 | 50.00 | C |
| ATOM | 5643 | C | PHE | D | 387 | 0.887 | 61.782 | 40.240 | 1.00 | 50.00 | C |
| ATOM | 5644 | O | PHE | D | 387 | 0.287 | 62.811 | 40.549 | 1.00 | 50.00 | O |
| ATOM | 5645 | CB | PHE | D | 387 | 3.397 | 61.989 | 40.199 | 1.00 | 50.00 | C |
| ATOM | 5646 | CG | PHE | D | 387 | 3.381 | 63.470 | 39.879 | 1.00 | 50.00 | C |
| ATOM | 5647 | CD1 | PHE | D | 387 | 3.694 | 64.395 | 40.811 | 1.00 | 50.00 | C |
| ATOM | 5648 | CD2 | PHE | D | 387 | 2.915 | 63.877 | 38.680 | 1.00 | 50.00 | C |
| ATOM | 5649 | CE1 | PHE | D | 387 | 3.545 | 65.709 | 40.549 | 1.00 | 50.00 | C |
| ATOM | 5650 | CE2 | PHE | D | 387 | 2.819 | 65.191 | 38.400 | 1.00 | 50.00 | C |
| ATOM | 5651 | CZ | PHE | D | 387 | 3.108 | 66.112 | 39.339 | 1.00 | 50.00 | C |
| ATOM | 5652 | N | ASN | D | 388 | 0.424 | 60.977 | 39.297 | 1.00 | 50.00 | N |
| ATOM | 5653 | CA | ASN | D | 388 | −0.962 | 60.969 | 38.821 | 1.00 | 50.00 | C |
| ATOM | 5654 | C | ASN | D | 388 | −2.115 | 61.212 | 39.796 | 1.00 | 50.00 | C |
| ATOM | 5655 | O | ASN | D | 388 | −3.040 | 61.913 | 39.386 | 1.00 | 50.00 | O |
| ATOM | 5656 | CB | ASN | D | 388 | −1.170 | 59.729 | 37.951 | 1.00 | 50.00 | C |
| ATOM | 5657 | CG | ASN | D | 388 | −1.067 | 58.350 | 38.589 | 1.00 | 50.00 | C |
| ATOM | 5658 | OD1 | ASN | D | 388 | −2.065 | 57.839 | 39.093 | 1.00 | 50.00 | O |
| ATOM | 5659 | ND2 | ASN | D | 388 | 0.104 | 57.756 | 38.733 | 1.00 | 50.00 | N |
| ATOM | 5660 | N | PRO | D | 389 | −2.148 | 60.746 | 41.037 | 1.00 | 50.00 | N |
| ATOM | 5661 | CA | PRO | D | 389 | −3.161 | 61.048 | 42.053 | 1.00 | 50.00 | C |
| ATOM | 5662 | C | PRO | D | 389 | −3.192 | 62.428 | 42.693 | 1.00 | 50.00 | C |
| ATOM | 5663 | O | PRO | D | 389 | −4.256 | 62.950 | 43.024 | 1.00 | 50.00 | O |
| ATOM | 5664 | CB | PRO | D | 389 | −2.859 | 60.143 | 43.246 | 1.00 | 50.00 | C |
| ATOM | 5665 | CG | PRO | D | 389 | −2.126 | 59.047 | 42.486 | 1.00 | 50.00 | C |
| ATOM | 5666 | CD | PRO | D | 389 | −1.169 | 59.809 | 41.583 | 1.00 | 50.00 | C |
| ATOM | 5667 | N | LYS | D | 390 | −1.998 | 62.964 | 42.881 | 1.00 | 50.00 | N |
| ATOM | 5668 | CA | LYS | D | 390 | −1.769 | 64.359 | 43.249 | 1.00 | 50.00 | C |
| ATOM | 5669 | C | LYS | D | 390 | −2.253 | 65.405 | 42.252 | 1.00 | 50.00 | C |
| ATOM | 5670 | O | LYS | D | 390 | −2.055 | 66.574 | 42.581 | 1.00 | 50.00 | O |
| ATOM | 5671 | CB | LYS | D | 390 | −0.269 | 64.493 | 43.513 | 1.00 | 50.00 | C |
| ATOM | 5672 | CG | LYS | D | 390 | 0.263 | 63.670 | 44.686 | 1.00 | 50.00 | C |
| ATOM | 5673 | CD | LYS | D | 390 | 1.713 | 64.022 | 45.009 | 1.00 | 50.00 | C |
| ATOM | 5674 | CE | LYS | D | 390 | 2.246 | 63.193 | 46.175 | 1.00 | 50.00 | C |
| ATOM | 5675 | NZ | LYS | D | 390 | 3.610 | 63.628 | 46.505 | 1.00 | 50.00 | N |
| ATOM | 5676 | N | TYR | D | 391 | −2.885 | 65.028 | 41.149 | 1.00 | 50.00 | N |
| ATOM | 5677 | CA | TYR | D | 391 | −3.626 | 65.846 | 40.188 | 1.00 | 50.00 | C |
| ATOM | 5678 | C | TYR | D | 391 | −3.237 | 65.632 | 38.729 | 1.00 | 50.00 | C |
| ATOM | 5679 | O | TYR | D | 391 | −4.048 | 66.007 | 37.883 | 1.00 | 50.00 | O |
| ATOM | 5680 | CB | TYR | D | 391 | −3.832 | 67.307 | 40.589 | 1.00 | 50.00 | C |
| ATOM | 5681 | CG | TYR | D | 391 | −4.541 | 68.259 | 39.654 | 1.00 | 50.00 | C |
| ATOM | 5682 | CD1 | TYR | D | 391 | −5.883 | 68.249 | 39.521 | 1.00 | 50.00 | C |
| ATOM | 5683 | CD2 | TYR | D | 391 | −3.791 | 69.071 | 38.879 | 1.00 | 50.00 | C |
| ATOM | 5684 | CE1 | TYR | D | 391 | −6.475 | 69.065 | 38.626 | 1.00 | 50.00 | C |
| ATOM | 5685 | CE2 | TYR | D | 391 | −4.383 | 69.888 | 37.986 | 1.00 | 50.00 | C |
| ATOM | 5686 | CZ | TYR | D | 391 | −5.726 | 69.891 | 37.867 | 1.00 | 50.00 | C |
| ATOM | 5687 | OH | TYR | D | 391 | −6.322 | 70.774 | 37.006 | 1.00 | 50.00 | O |
| ATOM | 5688 | N | ASP | D | 392 | −2.093 | 65.081 | 38.355 | 1.00 | 50.00 | N |
| ATOM | 5689 | CA | ASP | D | 392 | −1.623 | 65.118 | 36.970 | 1.00 | 50.00 | C |
| ATOM | 5690 | C | ASP | D | 392 | −2.460 | 64.265 | 36.022 | 1.00 | 50.00 | C |
| ATOM | 5691 | O | ASP | D | 392 | −3.512 | 63.716 | 36.349 | 1.00 | 50.00 | O |
| ATOM | 5692 | CB | ASP | D | 392 | −0.110 | 64.901 | 36.953 | 1.00 | 50.00 | C |
| ATOM | 5693 | CG | ASP | D | 392 | 0.620 | 65.487 | 35.752 | 1.00 | 50.00 | C |
| ATOM | 5694 | OD1 | ASP | D | 392 | 0.802 | 66.722 | 35.699 | 1.00 | 50.00 | O |
| ATOM | 5695 | OD2 | ASP | D | 392 | 1.027 | 64.720 | 34.853 | 1.00 | 50.00 | O |
| ATOM | 5696 | N | CYS | D | 393 | −2.013 | 64.284 | 34.780 | 1.00 | 50.00 | N |
| ATOM | 5697 | CA | CYS | D | 393 | −2.704 | 63.759 | 33.606 | 1.00 | 50.00 | C |
| ATOM | 5698 | C | CYS | D | 393 | −3.622 | 64.752 | 32.914 | 1.00 | 50.00 | C |
| ATOM | 5699 | O | CYS | D | 393 | −4.239 | 65.628 | 33.520 | 1.00 | 50.00 | O |
| ATOM | 5700 | CB | CYS | D | 393 | −3.263 | 62.340 | 33.665 | 1.00 | 50.00 | C |
| ATOM | 5701 | SG | CYS | D | 393 | −1.745 | 61.514 | 33.181 | 1.00 | 50.00 | S |
| ATOM | 5702 | N | LYS | D | 394 | −3.620 | 64.552 | 31.608 | 1.00 | 50.00 | N |
| ATOM | 5703 | CA | LYS | D | 394 | −4.354 | 65.411 | 30.683 | 1.00 | 50.00 | C |
| ATOM | 5704 | C | LYS | D | 394 | −5.358 | 64.518 | 29.973 | 1.00 | 50.00 | C |
| ATOM | 5705 | O | LYS | D | 394 | −5.163 | 63.902 | 28.926 | 1.00 | 50.00 | O |
| ATOM | 5706 | CB | LYS | D | 394 | −3.429 | 66.183 | 29.746 | 1.00 | 50.00 | C |
| ATOM | 5707 | CG | LYS | D | 394 | −2.599 | 67.174 | 30.558 | 1.00 | 50.00 | C |
| ATOM | 5708 | CD | LYS | D | 394 | −1.852 | 68.123 | 29.628 | 1.00 | 50.00 | C |
| ATOM | 5709 | CE | LYS | D | 394 | −1.012 | 69.068 | 30.481 | 1.00 | 50.00 | C |
| ATOM | 5710 | NZ | LYS | D | 394 | −0.185 | 69.891 | 29.587 | 1.00 | 50.00 | N |
| ATOM | 5711 | N | ILE | D | 395 | −6.487 | 64.565 | 30.654 | 1.00 | 50.00 | N |
| ATOM | 5712 | CA | ILE | D | 395 | −7.798 | 64.086 | 30.225 | 1.00 | 50.00 | C |
| ATOM | 5713 | C | ILE | D | 395 | −8.261 | 64.862 | 28.994 | 1.00 | 50.00 | C |
| ATOM | 5714 | O | ILE | D | 395 | −8.077 | 66.077 | 28.925 | 1.00 | 50.00 | O |
| ATOM | 5715 | CB | ILE | D | 395 | −8.579 | 64.233 | 31.537 | 1.00 | 50.00 | C |
| ATOM | 5716 | CG1 | ILE | D | 395 | −8.179 | 63.291 | 32.676 | 1.00 | 50.00 | C |
| ATOM | 5717 | CG2 | ILE | D | 395 | −10.055 | 63.994 | 31.241 | 1.00 | 50.00 | C |
| ATOM | 5718 | CD1 | ILE | D | 395 | −9.151 | 62.930 | 33.799 | 1.00 | 50.00 | C |
| ATOM | 5719 | N | MET | D | 396 | −8.879 | 64.191 | 28.035 | 1.00 | 50.00 | N |
| ATOM | 5720 | CA | MET | D | 396 | −9.520 | 64.821 | 26.879 | 1.00 | 50.00 | C |
| ATOM | 5721 | C | MET | D | 396 | −10.744 | 64.076 | 26.363 | 1.00 | 50.00 | C |
| ATOM | 5722 | O | MET | D | 396 | −11.803 | 64.692 | 26.267 | 1.00 | 50.00 | O |
| ATOM | 5723 | CB | MET | D | 396 | −8.599 | 65.087 | 25.687 | 1.00 | 50.00 | C |
| ATOM | 5724 | CG | MET | D | 396 | −7.915 | 66.450 | 25.735 | 1.00 | 50.00 | C |
| ATOM | 5725 | SD | MET | D | 396 | −6.880 | 66.663 | 24.278 | 1.00 | 50.00 | S |
| ATOM | 5726 | CE | MET | D | 396 | −6.173 | 68.242 | 24.776 | 1.00 | 50.00 | C |
| ATOM | 5727 | N | THR | D | 397 | −10.596 | 62.846 | 25.901 | 1.00 | 50.00 | N |
| ATOM | 5728 | CA | THR | D | 397 | −11.624 | 62.067 | 25.211 | 1.00 | 50.00 | C |
| ATOM | 5729 | C | THR | D | 397 | −10.937 | 60.761 | 24.841 | 1.00 | 50.00 | C |
| ATOM | 5730 | O | THR | D | 397 | −9.780 | 60.723 | 24.421 | 1.00 | 50.00 | O |
| ATOM | 5731 | CB | THR | D | 397 | −12.335 | 62.655 | 23.988 | 1.00 | 50.00 | C |
| ATOM | 5732 | OG1 | THR | D | 397 | −13.408 | 61.802 | 23.613 | 1.00 | 50.00 | O |
| ATOM | 5733 | CG2 | THR | D | 397 | −11.501 | 62.941 | 22.741 | 1.00 | 50.00 | C |
| ATOM | 5734 | N | SER | D | 398 | −11.701 | 59.684 | 24.928 | 1.00 | 50.00 | N |
| ATOM | 5735 | CA | SER | D | 398 | −11.233 | 58.398 | 24.417 | 1.00 | 50.00 | C |
| ATOM | 5736 | C | SER | D | 398 | −11.337 | 58.524 | 22.904 | 1.00 | 50.00 | C |
| ATOM | 5737 | O | SER | D | 398 | −12.031 | 59.351 | 22.312 | 1.00 | 50.00 | O |
| ATOM | 5738 | CB | SER | D | 398 | −11.942 | 57.181 | 25.007 | 1.00 | 50.00 | C |
| ATOM | 5739 | OG | SER | D | 398 | −13.348 | 57.229 | 24.803 | 1.00 | 50.00 | O |
| ATOM | 5740 | N | LYS | D | 399 | −10.611 | 57.628 | 22.265 | 1.00 | 50.00 | N |
| ATOM | 5741 | CA | LYS | D | 399 | −10.752 | 57.426 | 20.824 | 1.00 | 50.00 | C |
| ATOM | 5742 | C | LYS | D | 399 | −12.083 | 56.811 | 20.400 | 1.00 | 50.00 | C |
| ATOM | 5743 | O | LYS | D | 399 | −12.069 | 56.108 | 19.392 | 1.00 | 50.00 | O |
| ATOM | 5744 | CB | LYS | D | 399 | −9.511 | 56.600 | 20.481 | 1.00 | 50.00 | C |
| ATOM | 5745 | CG | LYS | D | 399 | −8.232 | 57.432 | 20.550 | 1.00 | 50.00 | C |
| ATOM | 5746 | CD | LYS | D | 399 | −6.971 | 56.687 | 20.123 | 1.00 | 50.00 | C |
| ATOM | 5747 | CE | LYS | D | 399 | −5.790 | 57.647 | 20.252 | 1.00 | 50.00 | C |
| ATOM | 5748 | NZ | LYS | D | 399 | −4.565 | 57.026 | 19.731 | 1.00 | 50.00 | N |
| ATOM | 5749 | N | THR | D | 400 | −13.165 | 56.856 | 21.166 | 1.00 | 50.00 | N |
| ATOM | 5750 | CA | THR | D | 400 | −13.984 | 55.669 | 21.419 | 1.00 | 50.00 | C |
| ATOM | 5751 | C | THR | D | 400 | −15.429 | 56.035 | 21.742 | 1.00 | 50.00 | C |
| ATOM | 5752 | O | THR | D | 400 | −15.647 | 56.679 | 22.768 | 1.00 | 50.00 | O |
| ATOM | 5753 | CB | THR | D | 400 | −13.324 | 54.602 | 22.299 | 1.00 | 50.00 | C |
| ATOM | 5754 | OG1 | THR | D | 400 | −13.430 | 54.844 | 23.693 | 1.00 | 50.00 | O |
| ATOM | 5755 | CG2 | THR | D | 400 | −11.838 | 54.349 | 22.047 | 1.00 | 50.00 | C |
| ATOM | 5756 | N | ASP | D | 401 | −16.405 | 55.662 | 20.926 | 1.00 | 50.00 | N |
| ATOM | 5757 | CA | ASP | D | 401 | −17.804 | 56.061 | 21.099 | 1.00 | 50.00 | C |
| ATOM | 5758 | C | ASP | D | 401 | −18.429 | 55.532 | 22.385 | 1.00 | 50.00 | C |
| ATOM | 5759 | O | ASP | D | 401 | −18.836 | 56.331 | 23.228 | 1.00 | 50.00 | O |
| ATOM | 5760 | CB | ASP | D | 401 | −18.714 | 55.804 | 19.895 | 1.00 | 50.00 | C |
| ATOM | 5761 | CG | ASP | D | 401 | −20.028 | 56.573 | 19.917 | 1.00 | 50.00 | C |
| ATOM | 5762 | OD1 | ASP | D | 401 | −20.008 | 57.809 | 19.734 | 1.00 | 50.00 | O |
| ATOM | 5763 | OD2 | ASP | D | 401 | −21.093 | 55.958 | 20.142 | 1.00 | 50.00 | O |
| ATOM | 5764 | N | VAL | D | 402 | −18.532 | 54.219 | 22.510 | 1.00 | 50.00 | N |
| ATOM | 5765 | CA | VAL | D | 402 | −19.114 | 53.534 | 23.664 | 1.00 | 50.00 | C |
| ATOM | 5766 | C | VAL | D | 402 | −18.319 | 52.238 | 23.767 | 1.00 | 50.00 | C |
| ATOM | 5767 | O | VAL | D | 402 | −18.102 | 51.512 | 22.797 | 1.00 | 50.00 | O |
| ATOM | 5768 | CB | VAL | D | 402 | −20.620 | 53.274 | 23.563 | 1.00 | 50.00 | C |
| ATOM | 5769 | CG1 | VAL | D | 402 | −21.198 | 52.384 | 24.664 | 1.00 | 50.00 | C |
| ATOM | 5770 | CG2 | VAL | D | 402 | −21.455 | 54.554 | 23.548 | 1.00 | 50.00 | C |
| ATOM | 5771 | N | SER | D | 403 | −17.977 | 51.962 | 25.024 | 1.00 | 50.00 | N |
| ATOM | 5772 | CA | SER | D | 403 | −17.473 | 50.698 | 25.550 | 1.00 | 50.00 | C |
| ATOM | 5773 | C | SER | D | 403 | −16.922 | 51.158 | 26.896 | 1.00 | 50.00 | C |
| ATOM | 5774 | O | SER | D | 403 | −15.699 | 51.189 | 27.036 | 1.00 | 50.00 | O |
| ATOM | 5775 | CB | SER | D | 403 | −16.448 | 49.962 | 24.683 | 1.00 | 50.00 | C |
| ATOM | 5776 | OG | SER | D | 403 | −16.176 | 48.647 | 25.145 | 1.00 | 50.00 | O |
| ATOM | 5777 | N | SER | D | 404 | −17.717 | 51.564 | 27.876 | 1.00 | 50.00 | N |
| ATOM | 5778 | CA | SER | D | 404 | −17.199 | 52.055 | 29.155 | 1.00 | 50.00 | C |
| ATOM | 5779 | C | SER | D | 404 | −16.929 | 50.888 | 30.098 | 1.00 | 50.00 | C |
| ATOM | 5780 | O | SER | D | 404 | −17.629 | 50.646 | 31.081 | 1.00 | 50.00 | O |
| ATOM | 5781 | CB | SER | D | 404 | −18.118 | 53.118 | 29.756 | 1.00 | 50.00 | C |
| ATOM | 5782 | OG | SER | D | 404 | −18.133 | 54.310 | 28.984 | 1.00 | 50.00 | O |
| ATOM | 5783 | N | SER | D | 405 | −15.925 | 50.116 | 29.717 | 1.00 | 50.00 | N |
| ATOM | 5784 | CA | SER | D | 405 | −15.913 | 48.654 | 29.749 | 1.00 | 50.00 | C |
| ATOM | 5785 | C | SER | D | 405 | −14.971 | 48.128 | 28.673 | 1.00 | 50.00 | C |
| ATOM | 5786 | O | SER | D | 405 | −15.065 | 48.492 | 27.502 | 1.00 | 50.00 | O |
| ATOM | 5787 | CB | SER | D | 405 | −17.232 | 47.880 | 29.765 | 1.00 | 50.00 | C |
| ATOM | 5788 | OG | SER | D | 405 | −17.893 | 47.864 | 28.507 | 1.00 | 50.00 | O |
| ATOM | 5789 | N | VAL | D | 406 | −14.053 | 47.261 | 29.066 | 1.00 | 50.00 | N |
| ATOM | 5790 | CA | VAL | D | 406 | −13.033 | 46.654 | 28.209 | 1.00 | 50.00 | C |
| ATOM | 5791 | C | VAL | D | 406 | −12.798 | 45.259 | 28.779 | 1.00 | 50.00 | C |
| ATOM | 5792 | O | VAL | D | 406 | −12.783 | 45.047 | 29.991 | 1.00 | 50.00 | O |
| ATOM | 5793 | CB | VAL | D | 406 | −11.745 | 47.481 | 28.149 | 1.00 | 50.00 | C |
| ATOM | 5794 | CG1 | VAL | D | 406 | −10.531 | 46.782 | 27.537 | 1.00 | 50.00 | C |
| ATOM | 5795 | CG2 | VAL | D | 406 | −11.904 | 48.800 | 27.395 | 1.00 | 50.00 | C |
| ATOM | 5796 | N | ILE | D | 407 | −12.473 | 44.327 | 27.898 | 1.00 | 50.00 | N |
| ATOM | 5797 | CA | ILE | D | 407 | −11.915 | 43.030 | 28.281 | 1.00 | 50.00 | C |
| ATOM | 5798 | C | ILE | D | 407 | −10.407 | 43.030 | 28.513 | 1.00 | 50.00 | C |
| ATOM | 5799 | O | ILE | D | 407 | −9.645 | 43.672 | 27.791 | 1.00 | 50.00 | O |
| ATOM | 5800 | CB | ILE | D | 407 | −12.402 | 41.954 | 27.307 | 1.00 | 50.00 | C |
| ATOM | 5801 | CG1 | ILE | D | 407 | −12.682 | 40.651 | 28.053 | 1.00 | 50.00 | C |
| ATOM | 5802 | CG2 | ILE | D | 407 | −11.497 | 41.725 | 26.097 | 1.00 | 50.00 | C |
| ATOM | 5803 | CD1 | ILE | D | 407 | −13.934 | 40.711 | 28.927 | 1.00 | 50.00 | C |
| ATOM | 5804 | N | THR | D | 408 | −9.971 | 42.209 | 29.454 | 1.00 | 50.00 | N |
| ATOM | 5805 | CA | THR | D | 408 | −8.564 | 41.965 | 29.768 | 1.00 | 50.00 | C |
| ATOM | 5806 | C | THR | D | 408 | −8.518 | 40.620 | 30.483 | 1.00 | 50.00 | C |
| ATOM | 5807 | O | THR | D | 408 | −9.123 | 40.495 | 31.548 | 1.00 | 50.00 | O |
| ATOM | 5808 | CB | THR | D | 408 | −7.993 | 43.065 | 30.665 | 1.00 | 50.00 | C |
| ATOM | 5809 | OG1 | THR | D | 408 | −7.901 | 44.282 | 29.940 | 1.00 | 50.00 | O |
| ATOM | 5810 | CG2 | THR | D | 408 | −6.620 | 42.805 | 31.281 | 1.00 | 50.00 | C |
| ATOM | 5811 | N | SER | D | 409 | −7.727 | 39.687 | 29.976 | 1.00 | 50.00 | N |
| ATOM | 5812 | CA | SER | D | 409 | −7.318 | 38.465 | 30.673 | 1.00 | 50.00 | C |
| ATOM | 5813 | C | SER | D | 409 | −8.452 | 37.769 | 31.416 | 1.00 | 50.00 | C |
| ATOM | 5814 | O | SER | D | 409 | −8.512 | 37.928 | 32.634 | 1.00 | 50.00 | O |
| ATOM | 5815 | CB | SER | D | 409 | −6.148 | 38.728 | 31.623 | 1.00 | 50.00 | C |
| ATOM | 5816 | OG | SER | D | 409 | −4.964 | 39.176 | 30.980 | 1.00 | 50.00 | O |
| ATOM | 5817 | N | LEU | D | 410 | −9.409 | 37.143 | 30.751 | 1.00 | 50.00 | N |
| ATOM | 5818 | CA | LEU | D | 410 | −10.581 | 36.552 | 31.401 | 1.00 | 50.00 | C |
| ATOM | 5819 | C | LEU | D | 410 | −11.554 | 37.446 | 32.165 | 1.00 | 50.00 | C |
| ATOM | 5820 | O | LEU | D | 410 | −12.177 | 37.005 | 33.130 | 1.00 | 50.00 | O |
| ATOM | 5821 | CB | LEU | D | 410 | −10.177 | 35.271 | 32.137 | 1.00 | 50.00 | C |
| ATOM | 5822 | CG | LEU | D | 410 | −9.451 | 35.260 | 33.485 | 1.00 | 50.00 | C |
| ATOM | 5823 | CD1 | LEU | D | 410 | −9.830 | 36.210 | 34.623 | 1.00 | 50.00 | C |
| ATOM | 5824 | CD2 | LEU | D | 410 | −9.605 | 33.862 | 34.080 | 1.00 | 50.00 | C |
| ATOM | 5825 | N | GLY | D | 411 | −11.723 | 38.716 | 31.838 | 1.00 | 50.00 | N |
| ATOM | 5826 | CA | GLY | D | 411 | −12.208 | 39.677 | 32.829 | 1.00 | 50.00 | C |
| ATOM | 5827 | C | GLY | D | 411 | −12.589 | 41.033 | 32.259 | 1.00 | 50.00 | C |
| ATOM | 5828 | O | GLY | D | 411 | −12.002 | 41.463 | 31.268 | 1.00 | 50.00 | O |
| ATOM | 5829 | N | ALA | D | 412 | −13.538 | 41.709 | 32.884 | 1.00 | 50.00 | N |
| ATOM | 5830 | CA | ALA | D | 412 | −14.138 | 42.918 | 32.320 | 1.00 | 50.00 | C |
| ATOM | 5831 | C | ALA | D | 412 | −13.935 | 44.104 | 33.253 | 1.00 | 50.00 | C |
| ATOM | 5832 | O | ALA | D | 412 | −14.730 | 44.297 | 34.171 | 1.00 | 50.00 | O |
| ATOM | 5833 | CB | ALA | D | 412 | −15.605 | 42.629 | 32.003 | 1.00 | 50.00 | C |
| ATOM | 5834 | N | ILE | D | 413 | −12.925 | 44.924 | 33.010 | 1.00 | 50.00 | N |
| ATOM | 5835 | CA | ILE | D | 413 | −12.729 | 46.218 | 33.668 | 1.00 | 50.00 | C |
| ATOM | 5836 | C | ILE | D | 413 | −13.762 | 47.201 | 33.129 | 1.00 | 50.00 | C |
| ATOM | 5837 | O | ILE | D | 413 | −13.795 | 47.403 | 31.916 | 1.00 | 50.00 | O |
| ATOM | 5838 | CB | ILE | D | 413 | −11.294 | 46.714 | 33.465 | 1.00 | 50.00 | C |
| ATOM | 5839 | CG1 | ILE | D | 413 | −10.291 | 45.944 | 34.326 | 1.00 | 50.00 | C |
| ATOM | 5840 | CG2 | ILE | D | 413 | −11.048 | 48.212 | 33.649 | 1.00 | 50.00 | C |
| ATOM | 5841 | CD1 | ILE | D | 413 | −9.097 | 45.417 | 33.536 | 1.00 | 50.00 | C |
| ATOM | 5842 | N | VAL | D | 414 | −14.560 | 47.809 | 33.991 | 1.00 | 50.00 | N |
| ATOM | 5843 | CA | VAL | D | 414 | −15.724 | 48.594 | 33.578 | 1.00 | 50.00 | C |
| ATOM | 5844 | C | VAL | D | 414 | −15.836 | 49.854 | 34.431 | 1.00 | 50.00 | C |
| ATOM | 5845 | O | VAL | D | 414 | −15.683 | 49.784 | 35.650 | 1.00 | 50.00 | O |
| ATOM | 5846 | CB | VAL | D | 414 | −16.996 | 47.752 | 33.435 | 1.00 | 50.00 | C |
| ATOM | 5847 | CG1 | VAL | D | 414 | −16.904 | 46.340 | 32.856 | 1.00 | 50.00 | C |
| ATOM | 5848 | CG2 | VAL | D | 414 | −17.785 | 47.572 | 34.729 | 1.00 | 50.00 | C |
| ATOM | 5849 | N | SER | D | 415 | −16.201 | 50.982 | 33.841 | 1.00 | 50.00 | N |
| ATOM | 5850 | CA | SER | D | 415 | −16.626 | 52.176 | 34.572 | 1.00 | 50.00 | C |
| ATOM | 5851 | C | SER | D | 415 | −18.068 | 52.018 | 35.042 | 1.00 | 50.00 | C |
| ATOM | 5852 | O | SER | D | 415 | −19.007 | 52.452 | 34.375 | 1.00 | 50.00 | O |
| ATOM | 5853 | CB | SER | D | 415 | −16.443 | 53.423 | 33.706 | 1.00 | 50.00 | C |
| ATOM | 5854 | OG | SER | D | 415 | −15.093 | 53.629 | 33.317 | 1.00 | 50.00 | O |
| ATOM | 5855 | N | CYS | D | 416 | −18.251 | 51.367 | 36.181 | 1.00 | 50.00 | N |
| ATOM | 5856 | CA | CYS | D | 416 | −19.564 | 50.994 | 36.708 | 1.00 | 50.00 | C |
| ATOM | 5857 | C | CYS | D | 416 | −20.477 | 52.157 | 37.070 | 1.00 | 50.00 | C |
| ATOM | 5858 | O | CYS | D | 416 | −21.533 | 52.319 | 36.460 | 1.00 | 50.00 | O |
| ATOM | 5859 | CB | CYS | D | 416 | −19.433 | 50.026 | 37.883 | 1.00 | 50.00 | C |
| ATOM | 5860 | SG | CYS | D | 416 | −19.096 | 48.366 | 37.277 | 1.00 | 50.00 | S |
| ATOM | 5861 | N | TYR | D | 417 | −20.061 | 52.968 | 38.027 | 1.00 | 50.00 | N |
| ATOM | 5862 | CA | TYR | D | 417 | −20.704 | 54.243 | 38.342 | 1.00 | 50.00 | C |
| ATOM | 5863 | C | TYR | D | 417 | −20.571 | 55.330 | 37.279 | 1.00 | 50.00 | C |
| ATOM | 5864 | O | TYR | D | 417 | −21.548 | 56.052 | 37.082 | 1.00 | 50.00 | O |
| ATOM | 5865 | CB | TYR | D | 417 | −20.169 | 54.642 | 39.717 | 1.00 | 50.00 | C |
| ATOM | 5866 | CG | TYR | D | 417 | −20.829 | 55.857 | 40.330 | 1.00 | 50.00 | C |
| ATOM | 5867 | CD1 | TYR | D | 417 | −22.053 | 55.756 | 40.889 | 1.00 | 50.00 | C |
| ATOM | 5868 | CD2 | TYR | D | 417 | −20.249 | 57.069 | 40.204 | 1.00 | 50.00 | C |
| ATOM | 5869 | CE1 | TYR | D | 417 | −22.706 | 56.863 | 41.296 | 1.00 | 50.00 | C |
| ATOM | 5870 | CE2 | TYR | D | 417 | −20.900 | 58.178 | 40.610 | 1.00 | 50.00 | C |
| ATOM | 5871 | CZ | TYR | D | 417 | −22.130 | 58.074 | 41.154 | 1.00 | 50.00 | C |
| ATOM | 5872 | OH | TYR | D | 417 | −22.801 | 59.204 | 41.540 | 1.00 | 50.00 | O |
| ATOM | 5873 | N | GLY | D | 418 | −19.434 | 55.470 | 36.617 | 1.00 | 50.00 | N |
| ATOM | 5874 | CA | GLY | D | 418 | −19.226 | 56.426 | 35.529 | 1.00 | 50.00 | C |
| ATOM | 5875 | C | GLY | D | 418 | −20.257 | 56.413 | 34.408 | 1.00 | 50.00 | C |
| ATOM | 5876 | O | GLY | D | 418 | −21.024 | 57.367 | 34.281 | 1.00 | 50.00 | O |
| ATOM | 5877 | N | LYS | D | 419 | −20.340 | 55.310 | 33.682 | 1.00 | 50.00 | N |
| ATOM | 5878 | CA | LYS | D | 419 | −21.457 | 55.026 | 32.781 | 1.00 | 50.00 | C |
| ATOM | 5879 | C | LYS | D | 419 | −22.821 | 54.846 | 33.441 | 1.00 | 50.00 | C |
| ATOM | 5880 | O | LYS | D | 419 | −23.729 | 55.555 | 33.008 | 1.00 | 50.00 | O |
| ATOM | 5881 | CB | LYS | D | 419 | −21.111 | 53.816 | 31.913 | 1.00 | 50.00 | C |
| ATOM | 5882 | CG | LYS | D | 419 | −22.036 | 53.580 | 30.719 | 1.00 | 50.00 | C |
| ATOM | 5883 | CD | LYS | D | 419 | −21.625 | 54.407 | 29.503 | 1.00 | 50.00 | C |
| ATOM | 5884 | CE | LYS | D | 419 | −22.547 | 54.281 | 28.294 | 1.00 | 50.00 | C |
| ATOM | 5885 | NZ | LYS | D | 419 | −21.958 | 55.039 | 27.181 | 1.00 | 50.00 | N |
| ATOM | 5886 | N | THR | D | 420 | −22.968 | 53.910 | 34.367 | 1.00 | 50.00 | N |
| ATOM | 5887 | CA | THR | D | 420 | −24.198 | 53.250 | 34.818 | 1.00 | 50.00 | C |
| ATOM | 5888 | C | THR | D | 420 | −24.183 | 51.729 | 34.667 | 1.00 | 50.00 | C |
| ATOM | 5889 | O | THR | D | 420 | −25.125 | 51.088 | 35.132 | 1.00 | 50.00 | O |
| ATOM | 5890 | CB | THR | D | 420 | −25.531 | 53.919 | 34.465 | 1.00 | 50.00 | C |
| ATOM | 5891 | OG1 | THR | D | 420 | −25.516 | 55.254 | 34.949 | 1.00 | 50.00 | O |
| ATOM | 5892 | CG2 | THR | D | 420 | −26.850 | 53.356 | 34.990 | 1.00 | 50.00 | C |
| ATOM | 5893 | N | LYS | D | 421 | −23.168 | 51.074 | 34.124 | 1.00 | 50.00 | N |
| ATOM | 5894 | CA | LYS | D | 421 | −23.163 | 49.684 | 33.664 | 1.00 | 50.00 | C |
| ATOM | 5895 | C | LYS | D | 421 | −23.914 | 48.597 | 34.427 | 1.00 | 50.00 | C |
| ATOM | 5896 | O | LYS | D | 421 | −24.828 | 47.993 | 33.868 | 1.00 | 50.00 | O |
| ATOM | 5897 | CB | LYS | D | 421 | −21.720 | 49.266 | 33.370 | 1.00 | 50.00 | C |
| ATOM | 5898 | CG | LYS | D | 421 | −20.985 | 49.868 | 32.175 | 1.00 | 50.00 | C |
| ATOM | 5899 | CD | LYS | D | 421 | −21.446 | 49.364 | 30.810 | 1.00 | 50.00 | C |
| ATOM | 5900 | CE | LYS | D | 421 | −20.698 | 50.043 | 29.664 | 1.00 | 50.00 | C |
| ATOM | 5901 | NZ | LYS | D | 421 | −21.391 | 49.841 | 28.384 | 1.00 | 50.00 | N |
| ATOM | 5902 | N | CYS | D | 422 | −23.514 | 48.213 | 35.628 | 1.00 | 50.00 | N |
| ATOM | 5903 | CA | CYS | D | 422 | −23.062 | 46.835 | 35.817 | 1.00 | 50.00 | C |
| ATOM | 5904 | C | CYS | D | 422 | −24.016 | 45.955 | 36.617 | 1.00 | 50.00 | C |
| ATOM | 5905 | O | CYS | D | 422 | −23.922 | 45.853 | 37.839 | 1.00 | 50.00 | O |
| ATOM | 5906 | CB | CYS | D | 422 | −21.711 | 46.899 | 36.525 | 1.00 | 50.00 | C |
| ATOM | 5907 | SG | CYS | D | 422 | −20.444 | 47.978 | 35.850 | 1.00 | 50.00 | S |
| ATOM | 5908 | N | THR | D | 423 | −24.902 | 45.244 | 35.940 | 1.00 | 50.00 | N |
| ATOM | 5909 | CA | THR | D | 423 | −25.765 | 44.234 | 36.555 | 1.00 | 50.00 | C |
| ATOM | 5910 | C | THR | D | 423 | −25.028 | 42.901 | 36.608 | 1.00 | 50.00 | C |
| ATOM | 5911 | O | THR | D | 423 | −24.652 | 42.358 | 35.571 | 1.00 | 50.00 | O |
| ATOM | 5912 | CB | THR | D | 423 | −27.080 | 44.180 | 35.774 | 1.00 | 50.00 | C |
| ATOM | 5913 | OG1 | THR | D | 423 | −27.784 | 45.400 | 35.955 | 1.00 | 50.00 | O |
| ATOM | 5914 | CG2 | THR | D | 423 | −28.040 | 43.052 | 36.149 | 1.00 | 50.00 | C |
| ATOM | 5915 | N | ALA | D | 424 | −24.888 | 42.331 | 37.794 | 1.00 | 50.00 | N |
| ATOM | 5916 | CA | ALA | D | 424 | −24.474 | 40.938 | 37.962 | 1.00 | 50.00 | C |
| ATOM | 5917 | C | ALA | D | 424 | −25.707 | 40.048 | 38.063 | 1.00 | 50.00 | C |
| ATOM | 5918 | O | ALA | D | 424 | −26.403 | 40.044 | 39.078 | 1.00 | 50.00 | O |
| ATOM | 5919 | CB | ALA | D | 424 | −23.561 | 40.842 | 39.182 | 1.00 | 50.00 | C |
| ATOM | 5920 | N | SER | D | 425 | −25.974 | 39.286 | 37.014 | 1.00 | 50.00 | N |
| ATOM | 5921 | CA | SER | D | 425 | −27.038 | 38.281 | 36.978 | 1.00 | 50.00 | C |
| ATOM | 5922 | C | SER | D | 425 | −26.913 | 37.052 | 37.876 | 1.00 | 50.00 | C |
| ATOM | 5923 | O | SER | D | 425 | −27.872 | 36.283 | 37.930 | 1.00 | 50.00 | O |
| ATOM | 5924 | CB | SER | D | 425 | −27.298 | 37.908 | 35.518 | 1.00 | 50.00 | C |
| ATOM | 5925 | OG | SER | D | 425 | −27.847 | 38.995 | 34.787 | 1.00 | 50.00 | O |
| ATOM | 5926 | N | ASN | D | 426 | −25.829 | 36.839 | 38.607 | 1.00 | 50.00 | N |
| ATOM | 5927 | CA | ASN | D | 426 | −25.678 | 35.744 | 39.567 | 1.00 | 50.00 | C |
| ATOM | 5928 | C | ASN | D | 426 | −26.556 | 35.963 | 40.795 | 1.00 | 50.00 | C |
| ATOM | 5929 | O | ASN | D | 426 | −27.056 | 37.049 | 41.084 | 1.00 | 50.00 | O |
| ATOM | 5930 | CB | ASN | D | 426 | −24.196 | 35.552 | 39.899 | 1.00 | 50.00 | C |
| ATOM | 5931 | CG | ASN | D | 426 | −23.827 | 34.279 | 40.652 | 1.00 | 50.00 | C |
| ATOM | 5932 | OD1 | ASN | D | 426 | −23.768 | 33.189 | 40.085 | 1.00 | 50.00 | O |
| ATOM | 5933 | ND2 | ASN | D | 426 | −23.605 | 34.349 | 41.952 | 1.00 | 50.00 | N |
| ATOM | 5934 | N | LYS | D | 427 | −26.741 | 34.882 | 41.533 | 1.00 | 50.00 | N |
| ATOM | 5935 | CA | LYS | D | 427 | −27.415 | 34.830 | 42.830 | 1.00 | 50.00 | C |
| ATOM | 5936 | C | LYS | D | 427 | −27.115 | 35.883 | 43.891 | 1.00 | 50.00 | C |
| ATOM | 5937 | O | LYS | D | 427 | −28.082 | 36.378 | 44.470 | 1.00 | 50.00 | O |
| ATOM | 5938 | CB | LYS | D | 427 | −27.200 | 33.429 | 43.403 | 1.00 | 50.00 | C |
| ATOM | 5939 | CG | LYS | D | 427 | −27.846 | 32.298 | 42.604 | 1.00 | 50.00 | C |
| ATOM | 5940 | CD | LYS | D | 427 | −27.712 | 30.942 | 43.292 | 1.00 | 50.00 | C |
| ATOM | 5941 | CE | LYS | D | 427 | −28.350 | 29.826 | 42.467 | 1.00 | 50.00 | C |
| ATOM | 5942 | NZ | LYS | D | 427 | −28.256 | 28.544 | 43.181 | 1.00 | 50.00 | N |
| ATOM | 5943 | N | ASN | D | 428 | −25.871 | 36.236 | 44.178 | 1.00 | 50.00 | N |
| ATOM | 5944 | CA | ASN | D | 428 | −25.515 | 37.146 | 45.270 | 1.00 | 50.00 | C |
| ATOM | 5945 | C | ASN | D | 428 | −25.917 | 38.616 | 45.161 | 1.00 | 50.00 | C |
| ATOM | 5946 | O | ASN | D | 428 | −25.424 | 39.434 | 45.937 | 1.00 | 50.00 | O |
| ATOM | 5947 | CB | ASN | D | 428 | −24.016 | 36.982 | 45.540 | 1.00 | 50.00 | C |
| ATOM | 5948 | CG | ASN | D | 428 | −23.460 | 35.645 | 46.013 | 1.00 | 50.00 | C |
| ATOM | 5949 | OD1 | ASN | D | 428 | −22.636 | 35.024 | 45.342 | 1.00 | 50.00 | O |
| ATOM | 5950 | ND2 | ASN | D | 428 | −23.840 | 35.177 | 47.187 | 1.00 | 50.00 | N |
| ATOM | 5951 | N | ARG | D | 429 | −26.782 | 39.036 | 44.249 | 1.00 | 50.00 | N |
| ATOM | 5952 | CA | ARG | D | 429 | −26.580 | 40.224 | 43.416 | 1.00 | 50.00 | C |
| ATOM | 5953 | C | ARG | D | 429 | −27.876 | 40.735 | 42.791 | 1.00 | 50.00 | C |
| ATOM | 5954 | O | ARG | D | 429 | −28.767 | 39.931 | 42.517 | 1.00 | 50.00 | O |
| ATOM | 5955 | CB | ARG | D | 429 | −25.627 | 39.893 | 42.266 | 1.00 | 50.00 | C |
| ATOM | 5956 | CG | ARG | D | 429 | −24.200 | 39.434 | 42.570 | 1.00 | 50.00 | C |
| ATOM | 5957 | CD | ARG | D | 429 | −23.380 | 40.330 | 43.496 | 1.00 | 50.00 | C |
| ATOM | 5958 | NE | ARG | D | 429 | −23.270 | 41.620 | 42.824 | 1.00 | 50.00 | N |
| ATOM | 5959 | CZ | ARG | D | 429 | −22.587 | 42.660 | 43.264 | 1.00 | 50.00 | C |
| ATOM | 5960 | NH1 | ARG | D | 429 | −21.834 | 42.706 | 44.347 | 1.00 | 50.00 | N |
| ATOM | 5961 | NH2 | ARG | D | 429 | −22.697 | 43.723 | 42.496 | 1.00 | 50.00 | N |
| ATOM | 5962 | N | GLY | D | 430 | −28.139 | 41.974 | 42.405 | 1.00 | 50.00 | N |
| ATOM | 5963 | CA | GLY | D | 430 | −27.348 | 43.173 | 42.684 | 1.00 | 50.00 | C |
| ATOM | 5964 | C | GLY | D | 430 | −26.708 | 43.877 | 41.493 | 1.00 | 50.00 | C |
| ATOM | 5965 | O | GLY | D | 430 | −26.272 | 43.228 | 40.543 | 1.00 | 50.00 | O |
| ATOM | 5966 | N | ILE | D | 431 | −26.587 | 45.193 | 41.583 | 1.00 | 50.00 | N |
| ATOM | 5967 | CA | ILE | D | 431 | −25.767 | 46.027 | 40.699 | 1.00 | 50.00 | C |
| ATOM | 5968 | C | ILE | D | 431 | −24.409 | 46.216 | 41.370 | 1.00 | 50.00 | C |
| ATOM | 5969 | O | ILE | D | 431 | −24.335 | 46.566 | 42.548 | 1.00 | 50.00 | O |
| ATOM | 5970 | CB | ILE | D | 431 | −26.417 | 47.365 | 40.327 | 1.00 | 50.00 | C |
| ATOM | 5971 | CG1 | ILE | D | 431 | −27.736 | 47.208 | 39.567 | 1.00 | 50.00 | C |
| ATOM | 5972 | CG2 | ILE | D | 431 | −25.505 | 48.309 | 39.539 | 1.00 | 50.00 | C |
| ATOM | 5973 | CD1 | ILE | D | 431 | −28.526 | 48.473 | 39.236 | 1.00 | 50.00 | C |
| ATOM | 5974 | N | ILE | D | 432 | −23.334 | 46.075 | 40.609 | 1.00 | 50.00 | N |
| ATOM | 5975 | CA | ILE | D | 432 | −21.972 | 46.383 | 41.050 | 1.00 | 50.00 | C |
| ATOM | 5976 | C | ILE | D | 432 | −21.701 | 47.869 | 40.855 | 1.00 | 50.00 | C |
| ATOM | 5977 | O | ILE | D | 432 | −21.513 | 48.336 | 39.732 | 1.00 | 50.00 | O |
| ATOM | 5978 | CB | ILE | D | 432 | −20.909 | 45.428 | 40.499 | 1.00 | 50.00 | C |
| ATOM | 5979 | CG1 | ILE | D | 432 | −19.619 | 45.459 | 41.318 | 1.00 | 50.00 | C |
| ATOM | 5980 | CG2 | ILE | D | 432 | −20.568 | 45.463 | 39.012 | 1.00 | 50.00 | C |
| ATOM | 5981 | CD1 | ILE | D | 432 | −19.422 | 44.218 | 42.183 | 1.00 | 50.00 | C |
| ATOM | 5982 | N | LYS | D | 433 | −21.715 | 48.603 | 41.955 | 1.00 | 50.00 | N |
| ATOM | 5983 | CA | LYS | D | 433 | −21.502 | 50.050 | 41.972 | 1.00 | 50.00 | C |
| ATOM | 5984 | C | LYS | D | 433 | −20.119 | 50.372 | 42.523 | 1.00 | 50.00 | C |
| ATOM | 5985 | O | LYS | D | 433 | −19.557 | 49.658 | 43.354 | 1.00 | 50.00 | O |
| ATOM | 5986 | CB | LYS | D | 433 | −22.610 | 50.731 | 42.777 | 1.00 | 50.00 | C |
| ATOM | 5987 | CG | LYS | D | 433 | −23.996 | 50.648 | 42.140 | 1.00 | 50.00 | C |
| ATOM | 5988 | CD | LYS | D | 433 | −25.058 | 51.444 | 42.895 | 1.00 | 50.00 | C |
| ATOM | 5989 | CE | LYS | D | 433 | −26.446 | 51.276 | 42.280 | 1.00 | 50.00 | C |
| ATOM | 5990 | NZ | LYS | D | 433 | −27.435 | 52.088 | 43.005 | 1.00 | 50.00 | N |
| ATOM | 5991 | N | THR | D | 434 | −19.582 | 51.483 | 42.049 | 1.00 | 50.00 | N |
| ATOM | 5992 | CA | THR | D | 434 | −18.266 | 51.993 | 42.434 | 1.00 | 50.00 | C |
| ATOM | 5993 | C | THR | D | 434 | −18.470 | 53.365 | 43.064 | 1.00 | 50.00 | C |
| ATOM | 5994 | O | THR | D | 434 | −18.976 | 54.289 | 42.428 | 1.00 | 50.00 | O |
| ATOM | 5995 | CB | THR | D | 434 | −17.358 | 51.984 | 41.203 | 1.00 | 50.00 | C |
| ATOM | 5996 | OG1 | THR | D | 434 | −17.133 | 50.635 | 40.819 | 1.00 | 50.00 | O |
| ATOM | 5997 | CG2 | THR | D | 434 | −15.974 | 52.607 | 41.374 | 1.00 | 50.00 | C |
| ATOM | 5998 | N | PHE | D | 435 | −18.002 | 53.543 | 44.289 | 1.00 | 50.00 | N |
| ATOM | 5999 | CA | PHE | D | 435 | −17.862 | 54.862 | 44.907 | 1.00 | 50.00 | C |
| ATOM | 6000 | C | PHE | D | 435 | −16.713 | 55.664 | 44.300 | 1.00 | 50.00 | C |
| ATOM | 6001 | O | PHE | D | 435 | −15.852 | 55.138 | 43.596 | 1.00 | 50.00 | O |
| ATOM | 6002 | CB | PHE | D | 435 | −17.801 | 54.649 | 46.422 | 1.00 | 50.00 | C |
| ATOM | 6003 | CG | PHE | D | 435 | −17.884 | 55.896 | 47.277 | 1.00 | 50.00 | C |
| ATOM | 6004 | CD1 | PHE | D | 435 | −19.033 | 56.602 | 47.336 | 1.00 | 50.00 | C |
| ATOM | 6005 | CD2 | PHE | D | 435 | −16.762 | 56.416 | 47.815 | 1.00 | 50.00 | C |
| ATOM | 6006 | CE1 | PHE | D | 435 | −19.047 | 57.841 | 47.868 | 1.00 | 50.00 | C |
| ATOM | 6007 | CE2 | PHE | D | 435 | −16.767 | 57.658 | 48.341 | 1.00 | 50.00 | C |
| ATOM | 6008 | CZ | PHE | D | 435 | −17.910 | 58.373 | 48.359 | 1.00 | 50.00 | C |
| ATOM | 6009 | N | SER | D | 436 | −16.667 | 56.957 | 44.582 | 1.00 | 50.00 | N |
| ATOM | 6010 | CA | SER | D | 436 | −15.527 | 57.831 | 44.303 | 1.00 | 50.00 | C |
| ATOM | 6011 | C | SER | D | 436 | −14.191 | 57.332 | 44.844 | 1.00 | 50.00 | C |
| ATOM | 6012 | O | SER | D | 436 | −14.143 | 56.688 | 45.892 | 1.00 | 50.00 | O |
| ATOM | 6013 | CB | SER | D | 436 | −15.815 | 59.213 | 44.888 | 1.00 | 50.00 | C |
| ATOM | 6014 | OG | SER | D | 436 | −16.927 | 59.833 | 44.257 | 1.00 | 50.00 | O |
| ATOM | 6015 | N | ASN | D | 437 | −13.122 | 57.606 | 44.113 | 1.00 | 50.00 | N |
| ATOM | 6016 | CA | ASN | D | 437 | −11.786 | 57.048 | 44.330 | 1.00 | 50.00 | C |
| ATOM | 6017 | C | ASN | D | 437 | −11.464 | 55.794 | 43.524 | 1.00 | 50.00 | C |
| ATOM | 6018 | O | ASN | D | 437 | −10.324 | 55.670 | 43.080 | 1.00 | 50.00 | O |
| ATOM | 6019 | CB | ASN | D | 437 | −11.222 | 56.967 | 45.750 | 1.00 | 50.00 | C |
| ATOM | 6020 | CG | ASN | D | 437 | −11.262 | 58.248 | 46.571 | 1.00 | 50.00 | C |
| ATOM | 6021 | OD1 | ASN | D | 437 | −11.941 | 58.330 | 47.594 | 1.00 | 50.00 | O |
| ATOM | 6022 | ND2 | ASN | D | 437 | −10.562 | 59.283 | 46.144 | 1.00 | 50.00 | N |
| ATOM | 6023 | N | GLY | D | 438 | −12.404 | 54.894 | 43.285 | 1.00 | 50.00 | N |
| ATOM | 6024 | CA | GLY | D | 438 | −12.252 | 53.815 | 42.309 | 1.00 | 50.00 | C |
| ATOM | 6025 | C | GLY | D | 438 | −11.837 | 54.160 | 40.883 | 1.00 | 50.00 | C |
| ATOM | 6026 | O | GLY | D | 438 | −10.920 | 53.489 | 40.410 | 1.00 | 50.00 | O |
| ATOM | 6027 | N | CYS | D | 439 | −12.482 | 55.088 | 40.190 | 1.00 | 50.00 | N |
| ATOM | 6028 | CA | CYS | D | 439 | −12.467 | 55.225 | 38.729 | 1.00 | 50.00 | C |
| ATOM | 6029 | C | CYS | D | 439 | −13.289 | 54.144 | 38.031 | 1.00 | 50.00 | C |
| ATOM | 6030 | O | CYS | D | 439 | −14.097 | 54.444 | 37.153 | 1.00 | 50.00 | O |
| ATOM | 6031 | CB | CYS | D | 439 | −11.157 | 55.344 | 37.942 | 1.00 | 50.00 | C |
| ATOM | 6032 | SG | CYS | D | 439 | −10.316 | 56.900 | 37.602 | 1.00 | 50.00 | S |
| ATOM | 6033 | N | ASP | D | 440 | −12.960 | 52.891 | 38.302 | 1.00 | 50.00 | N |
| ATOM | 6034 | CA | ASP | D | 440 | −13.116 | 51.747 | 37.405 | 1.00 | 50.00 | C |
| ATOM | 6035 | C | ASP | D | 440 | −13.120 | 50.518 | 38.305 | 1.00 | 50.00 | C |
| ATOM | 6036 | O | ASP | D | 440 | −12.214 | 50.292 | 39.108 | 1.00 | 50.00 | O |
| ATOM | 6037 | CB | ASP | D | 440 | −11.961 | 51.606 | 36.413 | 1.00 | 50.00 | C |
| ATOM | 6038 | CG | ASP | D | 440 | −11.919 | 52.606 | 35.267 | 1.00 | 50.00 | C |
| ATOM | 6039 | OD1 | ASP | D | 440 | −12.897 | 52.668 | 34.491 | 1.00 | 50.00 | O |
| ATOM | 6040 | OD2 | ASP | D | 440 | −10.906 | 53.322 | 35.114 | 1.00 | 50.00 | O |
| ATOM | 6041 | N | TYR | D | 441 | −14.131 | 49.689 | 38.117 | 1.00 | 50.00 | N |
| ATOM | 6042 | CA | TYR | D | 441 | −14.266 | 48.398 | 38.791 | 1.00 | 50.00 | C |
| ATOM | 6043 | C | TYR | D | 441 | −13.560 | 47.264 | 38.053 | 1.00 | 50.00 | C |
| ATOM | 6044 | O | TYR | D | 441 | −13.368 | 47.317 | 36.839 | 1.00 | 50.00 | O |
| ATOM | 6045 | CB | TYR | D | 441 | −15.769 | 48.178 | 38.971 | 1.00 | 50.00 | C |
| ATOM | 6046 | CG | TYR | D | 441 | −16.096 | 47.085 | 39.964 | 1.00 | 50.00 | C |
| ATOM | 6047 | CD1 | TYR | D | 441 | −15.797 | 47.231 | 41.272 | 1.00 | 50.00 | C |
| ATOM | 6048 | CD2 | TYR | D | 441 | −16.444 | 45.872 | 39.490 | 1.00 | 50.00 | C |
| ATOM | 6049 | CE1 | TYR | D | 441 | −15.771 | 46.157 | 42.086 | 1.00 | 50.00 | C |
| ATOM | 6050 | CE2 | TYR | D | 441 | −16.431 | 44.798 | 40.305 | 1.00 | 50.00 | C |
| ATOM | 6051 | CZ | TYR | D | 441 | −16.072 | 44.938 | 41.597 | 1.00 | 50.00 | C |
| ATOM | 6052 | OH | TYR | D | 441 | −16.083 | 43.887 | 42.474 | 1.00 | 50.00 | O |
| ATOM | 6053 | N | VAL | D | 442 | −13.206 | 46.215 | 38.779 | 1.00 | 50.00 | N |
| ATOM | 6054 | CA | VAL | D | 442 | −12.593 | 45.000 | 38.240 | 1.00 | 50.00 | C |
| ATOM | 6055 | C | VAL | D | 442 | −13.597 | 44.092 | 37.536 | 1.00 | 50.00 | C |
| ATOM | 6056 | O | VAL | D | 442 | −13.417 | 43.879 | 36.338 | 1.00 | 50.00 | O |
| ATOM | 6057 | CB | VAL | D | 442 | −11.694 | 44.323 | 39.280 | 1.00 | 50.00 | C |
| ATOM | 6058 | CG1 | VAL | D | 442 | −11.206 | 42.925 | 38.899 | 1.00 | 50.00 | C |
| ATOM | 6059 | CG2 | VAL | D | 442 | −10.447 | 45.145 | 39.604 | 1.00 | 50.00 | C |
| ATOM | 6060 | N | SER | D | 443 | −14.552 | 43.479 | 38.217 | 1.00 | 50.00 | N |
| ATOM | 6061 | CA | SER | D | 443 | −15.495 | 42.495 | 37.682 | 1.00 | 50.00 | C |
| ATOM | 6062 | C | SER | D | 443 | −14.819 | 41.261 | 37.098 | 1.00 | 50.00 | C |
| ATOM | 6063 | O | SER | D | 443 | −14.643 | 41.158 | 35.884 | 1.00 | 50.00 | O |
| ATOM | 6064 | CB | SER | D | 443 | −16.594 | 43.013 | 36.753 | 1.00 | 50.00 | C |
| ATOM | 6065 | OG | SER | D | 443 | −17.593 | 43.768 | 37.422 | 1.00 | 50.00 | O |
| ATOM | 6066 | N | ASN | D | 444 | −14.431 | 40.339 | 37.962 | 1.00 | 50.00 | N |
| ATOM | 6067 | CA | ASN | D | 444 | −13.834 | 39.065 | 37.561 | 1.00 | 50.00 | C |
| ATOM | 6068 | C | ASN | D | 444 | −14.680 | 37.907 | 38.076 | 1.00 | 50.00 | C |
| ATOM | 6069 | O | ASN | D | 444 | −15.697 | 38.081 | 38.748 | 1.00 | 50.00 | O |
| ATOM | 6070 | CB | ASN | D | 444 | −12.365 | 39.041 | 37.992 | 1.00 | 50.00 | C |
| ATOM | 6071 | CG | ASN | D | 444 | −11.345 | 39.884 | 37.240 | 1.00 | 50.00 | C |
| ATOM | 6072 | OD1 | ASN | D | 444 | −10.279 | 40.201 | 37.765 | 1.00 | 50.00 | O |
| ATOM | 6073 | ND2 | ASN | D | 444 | −11.592 | 40.249 | 35.995 | 1.00 | 50.00 | N |
| ATOM | 6074 | N | LYS | D | 445 | −14.219 | 36.708 | 37.759 | 1.00 | 50.00 | N |
| ATOM | 6075 | CA | LYS | D | 445 | −14.546 | 35.451 | 38.431 | 1.00 | 50.00 | C |
| ATOM | 6076 | C | LYS | D | 445 | −15.041 | 35.566 | 39.871 | 1.00 | 50.00 | C |
| ATOM | 6077 | O | LYS | D | 445 | −16.257 | 35.538 | 40.063 | 1.00 | 50.00 | O |
| ATOM | 6078 | CB | LYS | D | 445 | −13.373 | 34.490 | 38.227 | 1.00 | 50.00 | C |
| ATOM | 6079 | CG | LYS | D | 445 | −13.185 | 33.897 | 36.830 | 1.00 | 50.00 | C |
| ATOM | 6080 | CD | LYS | D | 445 | −12.364 | 32.609 | 36.782 | 1.00 | 50.00 | C |
| ATOM | 6081 | CE | LYS | D | 445 | −12.381 | 31.923 | 35.417 | 1.00 | 50.00 | C |
| ATOM | 6082 | NZ | LYS | D | 445 | −11.239 | 31.024 | 35.197 | 1.00 | 50.00 | N |
| ATOM | 6083 | N | GLY | D | 446 | −14.187 | 35.767 | 40.861 | 1.00 | 50.00 | N |
| ATOM | 6084 | CA | GLY | D | 446 | −14.583 | 35.812 | 42.269 | 1.00 | 50.00 | C |
| ATOM | 6085 | C | GLY | D | 446 | −15.667 | 36.776 | 42.737 | 1.00 | 50.00 | C |
| ATOM | 6086 | O | GLY | D | 446 | −16.505 | 36.375 | 43.544 | 1.00 | 50.00 | O |
| ATOM | 6087 | N | VAL | D | 447 | −15.694 | 37.997 | 42.229 | 1.00 | 50.00 | N |
| ATOM | 6088 | CA | VAL | D | 447 | −16.767 | 38.968 | 42.455 | 1.00 | 50.00 | C |
| ATOM | 6089 | C | VAL | D | 447 | −18.116 | 38.472 | 41.941 | 1.00 | 50.00 | C |
| ATOM | 6090 | O | VAL | D | 447 | −19.041 | 38.258 | 42.724 | 1.00 | 50.00 | O |
| ATOM | 6091 | CB | VAL | D | 447 | −16.353 | 40.338 | 41.906 | 1.00 | 50.00 | C |
| ATOM | 6092 | CG1 | VAL | D | 447 | −17.480 | 41.370 | 41.874 | 1.00 | 50.00 | C |
| ATOM | 6093 | CG2 | VAL | D | 447 | −15.142 | 40.955 | 42.606 | 1.00 | 50.00 | C |
| ATOM | 6094 | N | ASP | D | 448 | −18.229 | 38.374 | 40.627 | 1.00 | 50.00 | N |
| ATOM | 6095 | CA | ASP | D | 448 | −19.476 | 38.191 | 39.886 | 1.00 | 50.00 | C |
| ATOM | 6096 | C | ASP | D | 448 | −19.254 | 37.037 | 38.915 | 1.00 | 50.00 | C |
| ATOM | 6097 | O | ASP | D | 448 | −18.187 | 36.867 | 38.326 | 1.00 | 50.00 | O |
| ATOM | 6098 | CB | ASP | D | 448 | −19.903 | 39.471 | 39.166 | 1.00 | 50.00 | C |
| ATOM | 6099 | CG | ASP | D | 448 | −18.914 | 40.062 | 38.172 | 1.00 | 50.00 | C |
| ATOM | 6100 | OD1 | ASP | D | 448 | −17.936 | 40.705 | 38.609 | 1.00 | 50.00 | O |
| ATOM | 6101 | OD2 | ASP | D | 448 | −19.074 | 39.837 | 36.953 | 1.00 | 50.00 | O |
| ATOM | 6102 | N | THR | D | 449 | −20.303 | 36.260 | 38.712 | 1.00 | 50.00 | N |
| ATOM | 6103 | CA | THR | D | 449 | −20.287 | 35.119 | 37.796 | 1.00 | 50.00 | C |
| ATOM | 6104 | C | THR | D | 449 | −20.864 | 35.397 | 36.413 | 1.00 | 50.00 | C |
| ATOM | 6105 | O | THR | D | 449 | −20.559 | 34.602 | 35.525 | 1.00 | 50.00 | O |
| ATOM | 6106 | CB | THR | D | 449 | −20.921 | 33.945 | 38.544 | 1.00 | 50.00 | C |
| ATOM | 6107 | OG1 | THR | D | 449 | −20.136 | 33.598 | 39.676 | 1.00 | 50.00 | O |
| ATOM | 6108 | CG2 | THR | D | 449 | −21.182 | 32.657 | 37.767 | 1.00 | 50.00 | C |
| ATOM | 6109 | N | VAL | D | 450 | −21.688 | 36.407 | 36.179 | 1.00 | 50.00 | N |
| ATOM | 6110 | CA | VAL | D | 450 | −22.388 | 36.621 | 34.912 | 1.00 | 50.00 | C |
| ATOM | 6111 | C | VAL | D | 450 | −22.631 | 38.124 | 34.802 | 1.00 | 50.00 | C |
| ATOM | 6112 | O | VAL | D | 450 | −23.676 | 38.623 | 35.219 | 1.00 | 50.00 | O |
| ATOM | 6113 | CB | VAL | D | 450 | −23.625 | 35.734 | 34.732 | 1.00 | 50.00 | C |
| ATOM | 6114 | CG1 | VAL | D | 450 | −23.398 | 34.243 | 34.481 | 1.00 | 50.00 | C |
| ATOM | 6115 | CG2 | VAL | D | 450 | −24.603 | 35.760 | 35.905 | 1.00 | 50.00 | C |
| ATOM | 6116 | N | SER | D | 451 | −21.709 | 38.892 | 34.241 | 1.00 | 50.00 | N |
| ATOM | 6117 | CA | SER | D | 451 | −21.887 | 40.320 | 33.968 | 1.00 | 50.00 | C |
| ATOM | 6118 | C | SER | D | 451 | −22.922 | 40.636 | 32.893 | 1.00 | 50.00 | C |
| ATOM | 6119 | O | SER | D | 451 | −23.214 | 39.825 | 32.016 | 1.00 | 50.00 | O |
| ATOM | 6120 | CB | SER | D | 451 | −20.551 | 40.952 | 33.580 | 1.00 | 50.00 | C |
| ATOM | 6121 | OG | SER | D | 451 | −19.635 | 41.009 | 34.663 | 1.00 | 50.00 | O |
| ATOM | 6122 | N | VAL | D | 452 | −23.487 | 41.830 | 32.972 | 1.00 | 50.00 | N |
| ATOM | 6123 | CA | VAL | D | 452 | −24.431 | 42.384 | 32.000 | 1.00 | 50.00 | C |
| ATOM | 6124 | C | VAL | D | 452 | −24.084 | 43.869 | 31.963 | 1.00 | 50.00 | C |
| ATOM | 6125 | O | VAL | D | 452 | −24.299 | 44.576 | 32.947 | 1.00 | 50.00 | O |
| ATOM | 6126 | CB | VAL | D | 452 | −25.901 | 42.095 | 32.321 | 1.00 | 50.00 | C |
| ATOM | 6127 | CG1 | VAL | D | 452 | −26.908 | 42.866 | 31.467 | 1.00 | 50.00 | C |
| ATOM | 6128 | CG2 | VAL | D | 452 | −26.281 | 40.618 | 32.219 | 1.00 | 50.00 | C |
| ATOM | 6129 | N | GLY | D | 453 | −23.590 | 44.355 | 30.836 | 1.00 | 50.00 | N |
| ATOM | 6130 | CA | GLY | D | 453 | −23.246 | 45.766 | 30.664 | 1.00 | 50.00 | C |
| ATOM | 6131 | C | GLY | D | 453 | −24.059 | 46.487 | 29.598 | 1.00 | 50.00 | C |
| ATOM | 6132 | O | GLY | D | 453 | −23.471 | 46.873 | 28.588 | 1.00 | 50.00 | O |
| ATOM | 6133 | N | ASN | D | 454 | −25.346 | 46.692 | 29.829 | 1.00 | 50.00 | N |
| ATOM | 6134 | CA | ASN | D | 454 | −26.251 | 47.457 | 28.967 | 1.00 | 50.00 | C |
| ATOM | 6135 | C | ASN | D | 454 | −26.142 | 47.126 | 27.482 | 1.00 | 50.00 | C |
| ATOM | 6136 | O | ASN | D | 454 | −25.433 | 47.863 | 26.797 | 1.00 | 50.00 | O |
| ATOM | 6137 | CB | ASN | D | 454 | −26.140 | 48.960 | 29.239 | 1.00 | 50.00 | C |
| ATOM | 6138 | CG | ASN | D | 454 | −26.643 | 49.549 | 30.550 | 1.00 | 50.00 | C |
| ATOM | 6139 | OD1 | ASN | D | 454 | −25.921 | 50.260 | 31.248 | 1.00 | 50.00 | O |
| ATOM | 6140 | ND2 | ASN | D | 454 | −27.902 | 49.343 | 30.891 | 1.00 | 50.00 | N |
| ATOM | 6141 | N | THR | D | 455 | −26.747 | 46.044 | 27.017 | 1.00 | 50.00 | N |
| ATOM | 6142 | CA | THR | D | 455 | −26.630 | 45.478 | 25.669 | 1.00 | 50.00 | C |
| ATOM | 6143 | C | THR | D | 455 | −26.183 | 44.019 | 25.717 | 1.00 | 50.00 | C |
| ATOM | 6144 | O | THR | D | 455 | −26.821 | 43.206 | 25.048 | 1.00 | 50.00 | O |
| ATOM | 6145 | CB | THR | D | 455 | −25.989 | 46.303 | 24.547 | 1.00 | 50.00 | C |
| ATOM | 6146 | OG1 | THR | D | 455 | −26.593 | 47.588 | 24.492 | 1.00 | 50.00 | O |
| ATOM | 6147 | CG2 | THR | D | 455 | −26.036 | 45.858 | 23.086 | 1.00 | 50.00 | C |
| ATOM | 6148 | N | LEU | D | 456 | −25.107 | 43.652 | 26.398 | 1.00 | 50.00 | N |
| ATOM | 6149 | CA | LEU | D | 456 | −24.446 | 42.351 | 26.272 | 1.00 | 50.00 | C |
| ATOM | 6150 | C | LEU | D | 456 | −24.479 | 41.596 | 27.595 | 1.00 | 50.00 | C |
| ATOM | 6151 | O | LEU | D | 456 | −24.456 | 42.189 | 28.672 | 1.00 | 50.00 | O |
| ATOM | 6152 | CB | LEU | D | 456 | −22.988 | 42.471 | 25.822 | 1.00 | 50.00 | C |
| ATOM | 6153 | CG | LEU | D | 456 | −22.703 | 42.844 | 24.367 | 1.00 | 50.00 | C |
| ATOM | 6154 | CD1 | LEU | D | 456 | −23.096 | 44.276 | 24.014 | 1.00 | 50.00 | C |
| ATOM | 6155 | CD2 | LEU | D | 456 | −21.218 | 42.723 | 24.041 | 1.00 | 50.00 | C |
| ATOM | 6156 | N | TYR | D | 457 | −24.452 | 40.277 | 27.495 | 1.00 | 50.00 | N |
| ATOM | 6157 | CA | TYR | D | 457 | −24.270 | 39.371 | 28.630 | 1.00 | 50.00 | C |
| ATOM | 6158 | C | TYR | D | 457 | −22.861 | 38.804 | 28.489 | 1.00 | 50.00 | C |
| ATOM | 6159 | O | TYR | D | 457 | −22.509 | 38.173 | 27.494 | 1.00 | 50.00 | O |
| ATOM | 6160 | CB | TYR | D | 457 | −25.473 | 38.429 | 28.743 | 1.00 | 50.00 | C |
| ATOM | 6161 | CG | TYR | D | 457 | −25.360 | 36.931 | 28.540 | 1.00 | 50.00 | C |
| ATOM | 6162 | CD1 | TYR | D | 457 | −24.775 | 36.439 | 27.428 | 1.00 | 50.00 | C |
| ATOM | 6163 | CD2 | TYR | D | 457 | −25.761 | 36.074 | 29.502 | 1.00 | 50.00 | C |
| ATOM | 6164 | CE1 | TYR | D | 457 | −24.436 | 35.137 | 27.352 | 1.00 | 50.00 | C |
| ATOM | 6165 | CE2 | TYR | D | 457 | −25.500 | 34.755 | 29.396 | 1.00 | 50.00 | C |
| ATOM | 6166 | CZ | TYR | D | 457 | −24.822 | 34.290 | 28.327 | 1.00 | 50.00 | C |
| ATOM | 6167 | OH | TYR | D | 457 | −24.441 | 32.977 | 28.235 | 1.00 | 50.00 | O |
| ATOM | 6168 | N | TYR | D | 458 | −22.024 | 39.027 | 29.487 | 1.00 | 50.00 | N |
| ATOM | 6169 | CA | TYR | D | 458 | −20.768 | 38.298 | 29.657 | 1.00 | 50.00 | C |
| ATOM | 6170 | C | TYR | D | 458 | −20.916 | 37.229 | 30.734 | 1.00 | 50.00 | C |
| ATOM | 6171 | O | TYR | D | 458 | −21.460 | 37.509 | 31.801 | 1.00 | 50.00 | O |
| ATOM | 6172 | CB | TYR | D | 458 | −19.675 | 39.345 | 29.888 | 1.00 | 50.00 | C |
| ATOM | 6173 | CG | TYR | D | 458 | −18.278 | 38.780 | 29.765 | 1.00 | 50.00 | C |
| ATOM | 6174 | CD1 | TYR | D | 458 | −17.996 | 38.310 | 28.533 | 1.00 | 50.00 | C |
| ATOM | 6175 | CD2 | TYR | D | 458 | −17.603 | 38.317 | 30.838 | 1.00 | 50.00 | C |
| ATOM | 6176 | CE1 | TYR | D | 458 | −17.218 | 37.220 | 28.406 | 1.00 | 50.00 | C |
| ATOM | 6177 | CE2 | TYR | D | 458 | −16.745 | 37.286 | 30.701 | 1.00 | 50.00 | C |
| ATOM | 6178 | CZ | TYR | D | 458 | −16.607 | 36.710 | 29.491 | 1.00 | 50.00 | C |
| ATOM | 6179 | OH | TYR | D | 458 | −16.130 | 35.443 | 29.309 | 1.00 | 50.00 | O |
| ATOM | 6180 | N | VAL | D | 459 | −20.437 | 36.017 | 30.503 | 1.00 | 50.00 | N |
| ATOM | 6181 | CA | VAL | D | 459 | −20.401 | 34.956 | 31.512 | 1.00 | 50.00 | C |
| ATOM | 6182 | C | VAL | D | 459 | −18.992 | 34.935 | 32.091 | 1.00 | 50.00 | C |
| ATOM | 6183 | O | VAL | D | 459 | −18.051 | 34.639 | 31.357 | 1.00 | 50.00 | O |
| ATOM | 6184 | CB | VAL | D | 459 | −20.842 | 33.605 | 30.940 | 1.00 | 50.00 | C |
| ATOM | 6185 | CG1 | VAL | D | 459 | −20.633 | 32.389 | 31.842 | 1.00 | 50.00 | C |
| ATOM | 6186 | CG2 | VAL | D | 459 | −22.304 | 33.581 | 30.497 | 1.00 | 50.00 | C |
| ATOM | 6187 | N | ASN | D | 460 | −18.834 | 35.181 | 33.381 | 1.00 | 50.00 | N |
| ATOM | 6188 | CA | ASN | D | 460 | −17.578 | 34.975 | 34.104 | 1.00 | 50.00 | C |
| ATOM | 6189 | C | ASN | D | 460 | −17.384 | 33.629 | 34.805 | 1.00 | 50.00 | C |
| ATOM | 6190 | O | ASN | D | 460 | −16.304 | 33.569 | 35.390 | 1.00 | 50.00 | O |
| ATOM | 6191 | CB | ASN | D | 460 | −17.271 | 36.180 | 35.001 | 1.00 | 50.00 | C |
| ATOM | 6192 | CG | ASN | D | 460 | −17.105 | 37.597 | 34.467 | 1.00 | 50.00 | C |
| ATOM | 6193 | OD1 | ASN | D | 460 | −18.012 | 38.420 | 34.580 | 1.00 | 50.00 | O |
| ATOM | 6194 | ND2 | ASN | D | 460 | −15.953 | 37.994 | 33.955 | 1.00 | 50.00 | N |
| ATOM | 6195 | N | LYS | D | 461 | −18.154 | 32.545 | 34.775 | 1.00 | 50.00 | N |
| ATOM | 6196 | CA | LYS | D | 461 | −17.747 | 31.223 | 35.274 | 1.00 | 50.00 | C |
| ATOM | 6197 | C | LYS | D | 461 | −18.260 | 29.948 | 34.608 | 1.00 | 50.00 | C |
| ATOM | 6198 | O | LYS | D | 461 | −19.471 | 29.743 | 34.538 | 1.00 | 50.00 | O |
| ATOM | 6199 | CB | LYS | D | 461 | −17.820 | 30.917 | 36.769 | 1.00 | 50.00 | C |
| ATOM | 6200 | CG | LYS | D | 461 | −16.854 | 31.775 | 37.579 | 1.00 | 50.00 | C |
| ATOM | 6201 | CD | LYS | D | 461 | −16.910 | 31.421 | 39.060 | 1.00 | 50.00 | C |
| ATOM | 6202 | CE | LYS | D | 461 | −15.904 | 32.347 | 39.734 | 1.00 | 50.00 | C |
| ATOM | 6203 | NZ | LYS | D | 461 | −16.130 | 32.421 | 41.183 | 1.00 | 50.00 | N |
| ATOM | 6204 | N | GLN | D | 462 | −17.362 | 29.082 | 34.156 | 1.00 | 50.00 | N |
| ATOM | 6205 | CA | GLN | D | 462 | −17.635 | 27.918 | 33.309 | 1.00 | 50.00 | C |
| ATOM | 6206 | C | GLN | D | 462 | −16.410 | 27.017 | 33.182 | 1.00 | 50.00 | C |
| ATOM | 6207 | O | GLN | D | 462 | −15.263 | 27.461 | 33.128 | 1.00 | 50.00 | O |
| ATOM | 6208 | CB | GLN | D | 462 | −18.071 | 28.171 | 31.863 | 1.00 | 50.00 | C |
| ATOM | 6209 | CG | GLN | D | 462 | −19.405 | 28.816 | 31.490 | 1.00 | 50.00 | C |
| ATOM | 6210 | CD | GLN | D | 462 | −20.659 | 28.204 | 32.095 | 1.00 | 50.00 | C |
| ATOM | 6211 | OE1 | GLN | D | 462 | −20.732 | 27.020 | 32.421 | 1.00 | 50.00 | O |
| ATOM | 6212 | NE2 | GLN | D | 462 | −21.675 | 29.029 | 32.267 | 1.00 | 50.00 | N |
| ATOM | 6213 | N | GLU | D | 463 | −16.696 | 25.735 | 33.019 | 1.00 | 50.00 | N |
| ATOM | 6214 | CA | GLU | D | 463 | −15.738 | 24.655 | 32.782 | 1.00 | 50.00 | C |
| ATOM | 6215 | C | GLU | D | 463 | −16.307 | 23.633 | 31.802 | 1.00 | 50.00 | C |
| ATOM | 6216 | O | GLU | D | 463 | −17.490 | 23.606 | 31.463 | 1.00 | 50.00 | O |
| ATOM | 6217 | CB | GLU | D | 463 | −15.387 | 24.032 | 34.135 | 1.00 | 50.00 | C |
| ATOM | 6218 | CG | GLU | D | 463 | −14.208 | 24.693 | 34.850 | 1.00 | 50.00 | C |
| ATOM | 6219 | CD | GLU | D | 463 | −14.142 | 24.529 | 36.361 | 1.00 | 50.00 | C |
| ATOM | 6220 | OE1 | GLU | D | 463 | −15.143 | 24.147 | 37.006 | 1.00 | 50.00 | O |
| ATOM | 6221 | OE2 | GLU | D | 463 | −13.066 | 24.817 | 36.929 | 1.00 | 50.00 | O |
| ATOM | 6222 | N | GLY | D | 464 | −15.430 | 22.750 | 31.353 | 1.00 | 50.00 | N |
| ATOM | 6223 | CA | GLY | D | 464 | −15.784 | 21.556 | 30.589 | 1.00 | 50.00 | C |
| ATOM | 6224 | C | GLY | D | 464 | −14.688 | 20.508 | 30.727 | 1.00 | 50.00 | C |
| ATOM | 6225 | O | GLY | D | 464 | −13.502 | 20.812 | 30.849 | 1.00 | 50.00 | O |
| ATOM | 6226 | N | LYS | D | 465 | −15.098 | 19.251 | 30.732 | 1.00 | 50.00 | N |
| ATOM | 6227 | CA | LYS | D | 465 | −14.203 | 18.108 | 30.921 | 1.00 | 50.00 | C |
| ATOM | 6228 | C | LYS | D | 465 | −14.629 | 17.000 | 29.965 | 1.00 | 50.00 | C |
| ATOM | 6229 | O | LYS | D | 465 | −15.813 | 16.769 | 29.720 | 1.00 | 50.00 | O |
| ATOM | 6230 | CB | LYS | D | 465 | −14.181 | 17.626 | 32.372 | 1.00 | 50.00 | C |
| ATOM | 6231 | CG | LYS | D | 465 | −13.605 | 18.617 | 33.383 | 1.00 | 50.00 | C |
| ATOM | 6232 | CD | LYS | D | 465 | −13.594 | 18.095 | 34.818 | 1.00 | 50.00 | C |
| ATOM | 6233 | CE | LYS | D | 465 | −13.126 | 19.159 | 35.809 | 1.00 | 50.00 | C |
| ATOM | 6234 | NZ | LYS | D | 465 | −13.105 | 18.633 | 37.182 | 1.00 | 50.00 | N |
| ATOM | 6235 | N | SER | D | 466 | −13.644 | 16.312 | 29.412 | 1.00 | 50.00 | N |
| ATOM | 6236 | CA | SER | D | 466 | −13.832 | 15.281 | 28.391 | 1.00 | 50.00 | C |
| ATOM | 6237 | C | SER | D | 466 | −12.609 | 14.374 | 28.339 | 1.00 | 50.00 | C |
| ATOM | 6238 | O | SER | D | 466 | −11.483 | 14.765 | 28.645 | 1.00 | 50.00 | O |
| ATOM | 6239 | CB | SER | D | 466 | −14.097 | 15.850 | 26.997 | 1.00 | 50.00 | C |
| ATOM | 6240 | OG | SER | D | 466 | −15.411 | 16.375 | 26.878 | 1.00 | 50.00 | O |
| ATOM | 6241 | N | LEU | D | 467 | −12.866 | 13.132 | 27.965 | 1.00 | 50.00 | N |
| ATOM | 6242 | CA | LEU | D | 467 | −11.866 | 12.069 | 27.862 | 1.00 | 50.00 | C |
| ATOM | 6243 | C | LEU | D | 467 | −11.983 | 11.510 | 26.447 | 1.00 | 50.00 | C |
| ATOM | 6244 | O | LEU | D | 467 | −13.050 | 11.078 | 26.013 | 1.00 | 50.00 | O |
| ATOM | 6245 | CB | LEU | D | 467 | −12.074 | 11.024 | 28.960 | 1.00 | 50.00 | C |
| ATOM | 6246 | CG | LEU | D | 467 | −11.747 | 11.354 | 30.419 | 1.00 | 50.00 | C |
| ATOM | 6247 | CD1 | LEU | D | 467 | −12.629 | 12.387 | 31.121 | 1.00 | 50.00 | C |
| ATOM | 6248 | CD2 | LEU | D | 467 | −11.798 | 10.091 | 31.276 | 1.00 | 50.00 | C |
| ATOM | 6249 | N | TYR | D | 468 | −10.882 | 11.524 | 25.713 | 1.00 | 50.00 | N |
| ATOM | 6250 | CA | TYR | D | 468 | −10.743 | 10.963 | 24.369 | 1.00 | 50.00 | C |
| ATOM | 6251 | C | TYR | D | 468 | −10.068 | 9.608 | 24.535 | 1.00 | 50.00 | C |
| ATOM | 6252 | O | TYR | D | 468 | −8.918 | 9.541 | 24.968 | 1.00 | 50.00 | O |
| ATOM | 6253 | CB | TYR | D | 468 | −9.915 | 11.902 | 23.489 | 1.00 | 50.00 | C |
| ATOM | 6254 | CG | TYR | D | 468 | −9.840 | 11.615 | 22.004 | 1.00 | 50.00 | C |
| ATOM | 6255 | CD1 | TYR | D | 468 | −10.280 | 12.549 | 21.135 | 1.00 | 50.00 | C |
| ATOM | 6256 | CD2 | TYR | D | 468 | −9.434 | 10.419 | 21.528 | 1.00 | 50.00 | C |
| ATOM | 6257 | CE1 | TYR | D | 468 | −10.434 | 12.249 | 19.829 | 1.00 | 50.00 | C |
| ATOM | 6258 | CE2 | TYR | D | 468 | −9.643 | 10.096 | 20.237 | 1.00 | 50.00 | C |
| ATOM | 6259 | CZ | TYR | D | 468 | −10.174 | 11.001 | 19.391 | 1.00 | 50.00 | C |
| ATOM | 6260 | OH | TYR | D | 468 | −10.573 | 10.617 | 18.139 | 1.00 | 50.00 | O |
| ATOM | 6261 | N | VAL | D | 469 | −10.751 | 8.526 | 24.201 | 1.00 | 50.00 | N |
| ATOM | 6262 | CA | VAL | D | 469 | −10.283 | 7.170 | 24.491 | 1.00 | 50.00 | C |
| ATOM | 6263 | C | VAL | D | 469 | −10.787 | 6.249 | 23.386 | 1.00 | 50.00 | C |
| ATOM | 6264 | O | VAL | D | 469 | −11.857 | 6.456 | 22.813 | 1.00 | 50.00 | O |
| ATOM | 6265 | CB | VAL | D | 469 | −10.556 | 6.676 | 25.917 | 1.00 | 50.00 | C |
| ATOM | 6266 | CG1 | VAL | D | 469 | −10.596 | 7.726 | 27.028 | 1.00 | 50.00 | C |
| ATOM | 6267 | CG2 | VAL | D | 469 | −11.800 | 5.820 | 26.153 | 1.00 | 50.00 | C |
| ATOM | 6268 | N | LYS | D | 470 | −10.076 | 5.154 | 23.175 | 1.00 | 50.00 | N |
| ATOM | 6269 | CA | LYS | D | 470 | −10.586 | 4.007 | 22.426 | 1.00 | 50.00 | C |
| ATOM | 6270 | C | LYS | D | 470 | −10.825 | 2.767 | 23.280 | 1.00 | 50.00 | C |
| ATOM | 6271 | O | LYS | D | 470 | −10.411 | 2.669 | 24.434 | 1.00 | 50.00 | O |
| ATOM | 6272 | CB | LYS | D | 470 | −9.663 | 3.805 | 21.225 | 1.00 | 50.00 | C |
| ATOM | 6273 | CG | LYS | D | 470 | −10.490 | 3.177 | 20.106 | 1.00 | 50.00 | C |
| ATOM | 6274 | CD | LYS | D | 470 | −9.773 | 3.199 | 18.764 | 1.00 | 50.00 | C |
| ATOM | 6275 | CE | LYS | D | 470 | −10.706 | 2.509 | 17.774 | 1.00 | 50.00 | C |
| ATOM | 6276 | NZ | LYS | D | 470 | −9.745 | 2.249 | 16.700 | 1.00 | 50.00 | N |
| ATOM | 6277 | N | GLY | D | 471 | −11.536 | 1.809 | 22.709 | 1.00 | 50.00 | N |
| ATOM | 6278 | CA | GLY | D | 471 | −11.572 | 0.425 | 23.178 | 1.00 | 50.00 | C |
| ATOM | 6279 | C | GLY | D | 471 | −11.479 | −0.515 | 21.984 | 1.00 | 50.00 | C |
| ATOM | 6280 | O | GLY | D | 471 | −12.431 | −0.579 | 21.207 | 1.00 | 50.00 | O |
| ATOM | 6281 | N | GLU | D | 472 | −10.397 | −1.268 | 21.861 | 1.00 | 50.00 | N |
| ATOM | 6282 | CA | GLU | D | 472 | −10.258 | −2.324 | 20.856 | 1.00 | 50.00 | C |
| ATOM | 6283 | C | GLU | D | 472 | −11.327 | −3.406 | 20.969 | 1.00 | 50.00 | C |
| ATOM | 6284 | O | GLU | D | 472 | −11.541 | −3.897 | 22.078 | 1.00 | 50.00 | O |
| ATOM | 6285 | CB | GLU | D | 472 | −8.857 | −2.937 | 20.802 | 1.00 | 50.00 | C |
| ATOM | 6286 | CG | GLU | D | 472 | −8.592 | −4.437 | 20.653 | 1.00 | 50.00 | C |
| ATOM | 6287 | CD | GLU | D | 472 | −8.676 | −5.207 | 21.962 | 1.00 | 50.00 | C |
| ATOM | 6288 | OE1 | GLU | D | 472 | −9.648 | −5.960 | 22.187 | 1.00 | 50.00 | O |
| ATOM | 6289 | OE2 | GLU | D | 472 | −7.743 | −5.076 | 22.783 | 1.00 | 50.00 | O |
| ATOM | 6290 | N | PRO | D | 473 | −11.893 | −3.879 | 19.870 | 1.00 | 50.00 | N |
| ATOM | 6291 | CA | PRO | D | 473 | −12.628 | −5.144 | 19.841 | 1.00 | 50.00 | C |
| ATOM | 6292 | C | PRO | D | 473 | −11.962 | −6.190 | 18.953 | 1.00 | 50.00 | C |
| ATOM | 6293 | O | PRO | D | 473 | −12.058 | −6.121 | 17.728 | 1.00 | 50.00 | O |
| ATOM | 6294 | CB | PRO | D | 473 | −13.971 | −4.619 | 19.338 | 1.00 | 50.00 | C |
| ATOM | 6295 | CG | PRO | D | 473 | −13.646 | −3.500 | 18.351 | 1.00 | 50.00 | C |
| ATOM | 6296 | CD | PRO | D | 473 | −12.256 | −3.010 | 18.751 | 1.00 | 50.00 | C |
| ATOM | 6297 | N | ILE | D | 474 | −11.317 | −7.182 | 19.546 | 1.00 | 50.00 | N |
| ATOM | 6298 | CA | ILE | D | 474 | −10.790 | −8.347 | 18.832 | 1.00 | 50.00 | C |
| ATOM | 6299 | C | ILE | D | 474 | −11.916 | −9.297 | 18.436 | 1.00 | 50.00 | C |
| ATOM | 6300 | O | ILE | D | 474 | −12.801 | −9.581 | 19.244 | 1.00 | 50.00 | O |
| ATOM | 6301 | CB | ILE | D | 474 | −9.663 | −9.064 | 19.582 | 1.00 | 50.00 | C |
| ATOM | 6302 | CG1 | ILE | D | 474 | −10.068 | −9.712 | 20.907 | 1.00 | 50.00 | C |
| ATOM | 6303 | CG2 | ILE | D | 474 | −8.409 | −8.206 | 19.748 | 1.00 | 50.00 | C |
| ATOM | 6304 | CD1 | ILE | D | 474 | −9.114 | −10.799 | 21.397 | 1.00 | 50.00 | C |
| ATOM | 6305 | N | ILE | D | 475 | −11.885 | −9.810 | 17.217 | 1.00 | 50.00 | N |
| ATOM | 6306 | CA | ILE | D | 475 | −12.831 | −10.820 | 16.737 | 1.00 | 50.00 | C |
| ATOM | 6307 | C | ILE | D | 475 | −12.013 | −12.095 | 16.556 | 1.00 | 50.00 | C |
| ATOM | 6308 | O | ILE | D | 475 | −11.134 | −12.149 | 15.696 | 1.00 | 50.00 | O |
| ATOM | 6309 | CB | ILE | D | 475 | −13.590 | −10.380 | 15.481 | 1.00 | 50.00 | C |
| ATOM | 6310 | CG1 | ILE | D | 475 | −14.420 | −9.108 | 15.664 | 1.00 | 50.00 | C |
| ATOM | 6311 | CG2 | ILE | D | 475 | −14.490 | −11.489 | 14.933 | 1.00 | 50.00 | C |
| ATOM | 6312 | CD1 | ILE | D | 475 | −14.868 | −8.414 | 14.379 | 1.00 | 50.00 | C |
| ATOM | 6313 | N | ASN | D | 476 | −12.341 | −13.129 | 17.315 | 1.00 | 50.00 | N |
| ATOM | 6314 | CA | ASN | D | 476 | −11.743 | −14.457 | 17.179 | 1.00 | 50.00 | C |
| ATOM | 6315 | C | ASN | D | 476 | −12.819 | −15.478 | 16.833 | 1.00 | 50.00 | C |
| ATOM | 6316 | O | ASN | D | 476 | −13.692 | −15.769 | 17.650 | 1.00 | 50.00 | O |
| ATOM | 6317 | CB | ASN | D | 476 | −10.997 | −14.868 | 18.449 | 1.00 | 50.00 | C |
| ATOM | 6318 | CG | ASN | D | 476 | −9.750 | −14.082 | 18.828 | 1.00 | 50.00 | C |
| ATOM | 6319 | OD1 | ASN | D | 476 | −9.601 | −13.672 | 19.978 | 1.00 | 50.00 | O |
| ATOM | 6320 | ND2 | ASN | D | 476 | −8.814 | −13.880 | 17.919 | 1.00 | 50.00 | N |
| ATOM | 6321 | N | PHE | D | 477 | −12.724 | −16.049 | 15.643 | 1.00 | 50.00 | N |
| ATOM | 6322 | CA | PHE | D | 477 | −13.506 | −17.215 | 15.231 | 1.00 | 50.00 | C |
| ATOM | 6323 | C | PHE | D | 477 | −12.591 | −18.434 | 15.213 | 1.00 | 50.00 | C |
| ATOM | 6324 | O | PHE | D | 477 | −11.567 | −18.445 | 14.529 | 1.00 | 50.00 | O |
| ATOM | 6325 | CB | PHE | D | 477 | −14.176 | −16.952 | 13.880 | 1.00 | 50.00 | C |
| ATOM | 6326 | CG | PHE | D | 477 | −15.149 | −18.021 | 13.428 | 1.00 | 50.00 | C |
| ATOM | 6327 | CD1 | PHE | D | 477 | −16.381 | −18.092 | 13.976 | 1.00 | 50.00 | C |
| ATOM | 6328 | CD2 | PHE | D | 477 | −14.768 | −18.957 | 12.534 | 1.00 | 50.00 | C |
| ATOM | 6329 | CE1 | PHE | D | 477 | −17.217 | −19.098 | 13.648 | 1.00 | 50.00 | C |
| ATOM | 6330 | CE2 | PHE | D | 477 | −15.600 | −19.964 | 12.202 | 1.00 | 50.00 | C |
| ATOM | 6331 | CZ | PHE | D | 477 | −16.825 | −20.036 | 12.762 | 1.00 | 50.00 | C |
| ATOM | 6332 | N | TYR | D | 478 | −12.996 | −19.471 | 15.928 | 1.00 | 50.00 | N |
| ATOM | 6333 | CA | TYR | D | 478 | −12.325 | −20.771 | 15.939 | 1.00 | 50.00 | C |
| ATOM | 6334 | C | TYR | D | 478 | −13.294 | −21.834 | 15.432 | 1.00 | 50.00 | C |
| ATOM | 6335 | O | TYR | D | 478 | −14.426 | −21.947 | 15.901 | 1.00 | 50.00 | O |
| ATOM | 6336 | CB | TYR | D | 478 | −11.792 | −21.068 | 17.341 | 1.00 | 50.00 | C |
| ATOM | 6337 | CG | TYR | D | 478 | −11.025 | −22.366 | 17.484 | 1.00 | 50.00 | C |
| ATOM | 6338 | CD1 | TYR | D | 478 | −9.774 | −22.455 | 16.985 | 1.00 | 50.00 | C |
| ATOM | 6339 | CD2 | TYR | D | 478 | −11.611 | −23.465 | 18.005 | 1.00 | 50.00 | C |
| ATOM | 6340 | CE1 | TYR | D | 478 | −9.125 | −23.636 | 16.997 | 1.00 | 50.00 | C |
| ATOM | 6341 | CE2 | TYR | D | 478 | −10.967 | −24.650 | 18.002 | 1.00 | 50.00 | C |
| ATOM | 6342 | CZ | TYR | D | 478 | −9.724 | −24.739 | 17.488 | 1.00 | 50.00 | C |
| ATOM | 6343 | OH | TYR | D | 478 | −9.075 | −25.944 | 17.425 | 1.00 | 50.00 | O |
| ATOM | 6344 | N | ASP | D | 479 | −12.795 | −22.663 | 14.530 | 1.00 | 50.00 | N |
| ATOM | 6345 | CA | ASP | D | 479 | −13.444 | −23.910 | 14.124 | 1.00 | 50.00 | C |
| ATOM | 6346 | C | ASP | D | 479 | −12.723 | −25.101 | 14.746 | 1.00 | 50.00 | C |
| ATOM | 6347 | O | ASP | D | 479 | −11.505 | −25.175 | 14.586 | 1.00 | 50.00 | O |
| ATOM | 6348 | CB | ASP | D | 479 | −13.556 | −24.032 | 12.603 | 1.00 | 50.00 | C |
| ATOM | 6349 | CG | ASP | D | 479 | −12.283 | −24.154 | 11.776 | 1.00 | 50.00 | C |
| ATOM | 6350 | OD1 | ASP | D | 479 | −11.593 | −23.133 | 11.570 | 1.00 | 50.00 | O |
| ATOM | 6351 | OD2 | ASP | D | 479 | −11.952 | −25.278 | 11.339 | 1.00 | 50.00 | O |
| ATOM | 6352 | N | PRO | D | 480 | −13.366 | −26.093 | 15.345 | 1.00 | 50.00 | N |
| ATOM | 6353 | CA | PRO | D | 480 | −12.759 | −27.408 | 15.540 | 1.00 | 50.00 | C |
| ATOM | 6354 | C | PRO | D | 480 | −12.708 | −28.159 | 14.214 | 1.00 | 50.00 | C |
| ATOM | 6355 | O | PRO | D | 480 | −13.706 | −28.652 | 13.690 | 1.00 | 50.00 | O |
| ATOM | 6356 | CB | PRO | D | 480 | −13.646 | −28.012 | 16.627 | 1.00 | 50.00 | C |
| ATOM | 6357 | CG | PRO | D | 480 | −15.036 | −27.430 | 16.383 | 1.00 | 50.00 | C |
| ATOM | 6358 | CD | PRO | D | 480 | −14.747 | −26.039 | 15.822 | 1.00 | 50.00 | C |
| ATOM | 6359 | N | LEU | D | 481 | −11.503 | −28.282 | 13.683 | 1.00 | 50.00 | N |
| ATOM | 6360 | CA | LEU | D | 481 | −11.052 | −29.485 | 12.983 | 1.00 | 50.00 | C |
| ATOM | 6361 | C | LEU | D | 481 | −11.094 | −30.674 | 13.935 | 1.00 | 50.00 | C |
| ATOM | 6362 | O | LEU | D | 481 | −10.342 | −30.733 | 14.908 | 1.00 | 50.00 | O |
| ATOM | 6363 | CB | LEU | D | 481 | −9.646 | −29.230 | 12.437 | 1.00 | 50.00 | C |
| ATOM | 6364 | CG | LEU | D | 481 | −9.563 | −28.390 | 11.162 | 1.00 | 50.00 | C |
| ATOM | 6365 | CD1 | LEU | D | 481 | −8.130 | −27.920 | 10.919 | 1.00 | 50.00 | C |
| ATOM | 6366 | CD2 | LEU | D | 481 | −10.224 | −29.064 | 9.959 | 1.00 | 50.00 | C |
| ATOM | 6367 | N | VAL | D | 482 | −12.064 | −31.543 | 13.702 | 1.00 | 50.00 | N |
| ATOM | 6368 | CA | VAL | D | 482 | −12.443 | −32.601 | 14.638 | 1.00 | 50.00 | C |
| ATOM | 6369 | C | VAL | D | 482 | −11.923 | −33.872 | 13.981 | 1.00 | 50.00 | C |
| ATOM | 6370 | O | VAL | D | 482 | −12.487 | −34.387 | 13.016 | 1.00 | 50.00 | O |
| ATOM | 6371 | CB | VAL | D | 482 | −13.935 | −32.714 | 14.963 | 1.00 | 50.00 | C |
| ATOM | 6372 | CG1 | VAL | D | 482 | −14.242 | −33.783 | 16.012 | 1.00 | 50.00 | C |
| ATOM | 6373 | CG2 | VAL | D | 482 | −14.555 | −31.424 | 15.496 | 1.00 | 50.00 | C |
| ATOM | 6374 | N | PHE | D | 483 | −10.889 | −34.413 | 14.602 | 1.00 | 50.00 | N |
| ATOM | 6375 | CA | PHE | D | 483 | −10.433 | −35.772 | 14.326 | 1.00 | 50.00 | C |
| ATOM | 6376 | C | PHE | D | 483 | −11.479 | −36.830 | 14.667 | 1.00 | 50.00 | C |
| ATOM | 6377 | O | PHE | D | 483 | −12.003 | −36.732 | 15.776 | 1.00 | 50.00 | O |
| ATOM | 6378 | CB | PHE | D | 483 | −9.117 | −35.968 | 15.083 | 1.00 | 50.00 | C |
| ATOM | 6379 | CG | PHE | D | 483 | −8.294 | −37.046 | 14.415 | 1.00 | 50.00 | C |
| ATOM | 6380 | CD1 | PHE | D | 483 | −7.700 | −36.696 | 13.256 | 1.00 | 50.00 | C |
| ATOM | 6381 | CD2 | PHE | D | 483 | −8.585 | −38.344 | 14.640 | 1.00 | 50.00 | C |
| ATOM | 6382 | CE1 | PHE | D | 483 | −7.510 | −37.608 | 12.283 | 1.00 | 50.00 | C |
| ATOM | 6383 | CE2 | PHE | D | 483 | −8.463 | −39.255 | 13.655 | 1.00 | 50.00 | C |
| ATOM | 6384 | CZ | PHE | D | 483 | −8.027 | −38.841 | 12.450 | 1.00 | 50.00 | C |
| ATOM | 6385 | N | PRO | D | 484 | −11.783 | −37.850 | 13.874 | 1.00 | 50.00 | N |
| ATOM | 6386 | CA | PRO | D | 484 | −12.539 | −39.021 | 14.328 | 1.00 | 50.00 | C |
| ATOM | 6387 | C | PRO | D | 484 | −11.774 | −39.963 | 15.254 | 1.00 | 50.00 | C |
| ATOM | 6388 | O | PRO | D | 484 | −11.473 | −41.104 | 14.904 | 1.00 | 50.00 | O |
| ATOM | 6389 | CB | PRO | D | 484 | −12.957 | −39.640 | 12.994 | 1.00 | 50.00 | C |
| ATOM | 6390 | CG | PRO | D | 484 | −11.790 | −39.349 | 12.055 | 1.00 | 50.00 | C |
| ATOM | 6391 | CD | PRO | D | 484 | −11.442 | −37.918 | 12.454 | 1.00 | 50.00 | C |
| ATOM | 6392 | N | SER | D | 485 | −11.437 | −39.492 | 16.445 | 1.00 | 50.00 | N |
| ATOM | 6393 | CA | SER | D | 485 | −10.464 | −40.117 | 17.343 | 1.00 | 50.00 | C |
| ATOM | 6394 | C | SER | D | 485 | −10.950 | −41.353 | 18.090 | 1.00 | 50.00 | C |
| ATOM | 6395 | O | SER | D | 485 | −10.187 | −42.311 | 18.207 | 1.00 | 50.00 | O |
| ATOM | 6396 | CB | SER | D | 485 | −9.902 | −39.082 | 18.319 | 1.00 | 50.00 | C |
| ATOM | 6397 | OG | SER | D | 485 | −8.798 | −38.375 | 17.774 | 1.00 | 50.00 | O |
| ATOM | 6398 | N | ASP | D | 486 | −12.172 | −41.343 | 18.597 | 1.00 | 50.00 | N |
| ATOM | 6399 | CA | ASP | D | 486 | −12.775 | −42.469 | 19.312 | 1.00 | 50.00 | C |
| ATOM | 6400 | C | ASP | D | 486 | −13.092 | −43.689 | 18.453 | 1.00 | 50.00 | C |
| ATOM | 6401 | O | ASP | D | 486 | −12.810 | −44.793 | 18.916 | 1.00 | 50.00 | O |
| ATOM | 6402 | CB | ASP | D | 486 | −13.986 | −41.985 | 20.111 | 1.00 | 50.00 | C |
| ATOM | 6403 | CG | ASP | D | 486 | −13.709 | −40.932 | 21.175 | 1.00 | 50.00 | C |
| ATOM | 6404 | OD1 | ASP | D | 486 | −13.143 | −41.273 | 22.236 | 1.00 | 50.00 | O |
| ATOM | 6405 | OD2 | ASP | D | 486 | −14.034 | −39.746 | 20.948 | 1.00 | 50.00 | O |
| ATOM | 6406 | N | GLU | D | 487 | −13.581 | −43.549 | 17.230 | 1.00 | 50.00 | N |
| ATOM | 6407 | CA | GLU | D | 487 | −13.549 | −44.606 | 16.217 | 1.00 | 50.00 | C |
| ATOM | 6408 | C | GLU | D | 487 | −12.150 | −45.130 | 15.912 | 1.00 | 50.00 | C |
| ATOM | 6409 | O | GLU | D | 487 | −11.965 | −46.342 | 16.007 | 1.00 | 50.00 | O |
| ATOM | 6410 | CB | GLU | D | 487 | −14.280 | −44.224 | 14.929 | 1.00 | 50.00 | C |
| ATOM | 6411 | CG | GLU | D | 487 | −14.505 | −45.340 | 13.907 | 1.00 | 50.00 | C |
| ATOM | 6412 | CD | GLU | D | 487 | −13.442 | −45.527 | 12.832 | 1.00 | 50.00 | C |
| ATOM | 6413 | OE1 | GLU | D | 487 | −12.373 | −46.113 | 13.107 | 1.00 | 50.00 | O |
| ATOM | 6414 | OE2 | GLU | D | 487 | −13.675 | −45.108 | 11.678 | 1.00 | 50.00 | O |
| ATOM | 6415 | N | PHE | D | 488 | −11.197 | −44.284 | 15.553 | 1.00 | 50.00 | N |
| ATOM | 6416 | CA | PHE | D | 488 | −9.802 | −44.664 | 15.331 | 1.00 | 50.00 | C |
| ATOM | 6417 | C | PHE | D | 488 | −9.174 | −45.520 | 16.426 | 1.00 | 50.00 | C |
| ATOM | 6418 | O | PHE | D | 488 | −8.668 | −46.598 | 16.117 | 1.00 | 50.00 | O |
| ATOM | 6419 | CB | PHE | D | 488 | −9.002 | −43.404 | 14.996 | 1.00 | 50.00 | C |
| ATOM | 6420 | CG | PHE | D | 488 | −7.566 | −43.653 | 14.585 | 1.00 | 50.00 | C |
| ATOM | 6421 | CD1 | PHE | D | 488 | −7.280 | −44.092 | 13.342 | 1.00 | 50.00 | C |
| ATOM | 6422 | CD2 | PHE | D | 488 | −6.583 | −43.568 | 15.505 | 1.00 | 50.00 | C |
| ATOM | 6423 | CE1 | PHE | D | 488 | −6.021 | −44.448 | 13.019 | 1.00 | 50.00 | C |
| ATOM | 6424 | CE2 | PHE | D | 488 | −5.317 | −43.897 | 15.179 | 1.00 | 50.00 | C |
| ATOM | 6425 | CZ | PHE | D | 488 | −5.037 | −44.329 | 13.933 | 1.00 | 50.00 | C |
| ATOM | 6426 | N | ASP | D | 489 | −9.239 | −45.094 | 17.677 | 1.00 | 50.00 | N |
| ATOM | 6427 | CA | ASP | D | 489 | −8.781 | −45.855 | 18.841 | 1.00 | 50.00 | C |
| ATOM | 6428 | C | ASP | D | 489 | −9.461 | −47.203 | 19.061 | 1.00 | 50.00 | C |
| ATOM | 6429 | O | ASP | D | 489 | −8.753 | −48.180 | 19.302 | 1.00 | 50.00 | O |
| ATOM | 6430 | CB | ASP | D | 489 | −8.854 | −44.964 | 20.083 | 1.00 | 50.00 | C |
| ATOM | 6431 | CG | ASP | D | 489 | −8.013 | −43.696 | 20.129 | 1.00 | 50.00 | C |
| ATOM | 6432 | OD1 | ASP | D | 489 | −7.066 | −43.533 | 19.329 | 1.00 | 50.00 | O |
| ATOM | 6433 | OD2 | ASP | D | 489 | −8.306 | −42.830 | 20.982 | 1.00 | 50.00 | O |
| ATOM | 6434 | N | ALA | D | 490 | −10.775 | −47.300 | 18.934 | 1.00 | 50.00 | N |
| ATOM | 6435 | CA | ALA | D | 490 | −11.511 | −48.562 | 18.851 | 1.00 | 50.00 | C |
| ATOM | 6436 | C | ALA | D | 490 | −11.152 | −49.523 | 17.722 | 1.00 | 50.00 | C |
| ATOM | 6437 | O | ALA | D | 490 | −11.018 | −50.716 | 17.994 | 1.00 | 50.00 | O |
| ATOM | 6438 | CB | ALA | D | 490 | −13.006 | −48.247 | 18.811 | 1.00 | 50.00 | C |
| ATOM | 6439 | N | SER | D | 491 | −10.975 | −49.057 | 16.496 | 1.00 | 50.00 | N |
| ATOM | 6440 | CA | SER | D | 491 | −10.430 | −49.836 | 15.383 | 1.00 | 50.00 | C |
| ATOM | 6441 | C | SER | D | 491 | −9.020 | −50.381 | 15.576 | 1.00 | 50.00 | C |
| ATOM | 6442 | O | SER | D | 491 | −8.778 | −51.538 | 15.235 | 1.00 | 50.00 | O |
| ATOM | 6443 | CB | SER | D | 491 | −10.499 | −49.031 | 14.086 | 1.00 | 50.00 | C |
| ATOM | 6444 | OG | SER | D | 491 | −11.848 | −48.816 | 13.697 | 1.00 | 50.00 | O |
| ATOM | 6445 | N | ILE | D | 492 | −8.107 | −49.612 | 16.145 | 1.00 | 50.00 | N |
| ATOM | 6446 | CA | ILE | D | 492 | −6.787 | −50.067 | 16.583 | 1.00 | 50.00 | C |
| ATOM | 6447 | C | ILE | D | 492 | −6.825 | −51.141 | 17.665 | 1.00 | 50.00 | C |
| ATOM | 6448 | O | ILE | D | 492 | −6.087 | −52.117 | 17.542 | 1.00 | 50.00 | O |
| ATOM | 6449 | CB | ILE | D | 492 | −5.920 | −48.850 | 16.917 | 1.00 | 50.00 | C |
| ATOM | 6450 | CG1 | ILE | D | 492 | −5.714 | −48.030 | 15.641 | 1.00 | 50.00 | C |
| ATOM | 6451 | CG2 | ILE | D | 492 | −4.642 | −49.234 | 17.666 | 1.00 | 50.00 | C |
| ATOM | 6452 | CD1 | ILE | D | 492 | −4.617 | −46.974 | 15.687 | 1.00 | 50.00 | C |
| ATOM | 6453 | N | SER | D | 493 | −7.611 | −50.977 | 18.716 | 1.00 | 50.00 | N |
| ATOM | 6454 | CA | SER | D | 493 | −7.900 | −52.046 | 19.672 | 1.00 | 50.00 | C |
| ATOM | 6455 | C | SER | D | 493 | −8.485 | −53.326 | 19.084 | 1.00 | 50.00 | C |
| ATOM | 6456 | O | SER | D | 493 | −8.064 | −54.400 | 19.512 | 1.00 | 50.00 | O |
| ATOM | 6457 | CB | SER | D | 493 | −8.754 | −51.451 | 20.791 | 1.00 | 50.00 | C |
| ATOM | 6458 | OG | SER | D | 493 | −8.843 | −52.352 | 21.885 | 1.00 | 50.00 | O |
| ATOM | 6459 | N | GLN | D | 494 | −9.355 | −53.255 | 18.089 | 1.00 | 50.00 | N |
| ATOM | 6460 | CA | GLN | D | 494 | −9.694 | −54.367 | 17.198 | 1.00 | 50.00 | C |
| ATOM | 6461 | C | GLN | D | 494 | −8.539 | −54.990 | 16.420 | 1.00 | 50.00 | C |
| ATOM | 6462 | O | GLN | D | 494 | −8.502 | −56.219 | 16.358 | 1.00 | 50.00 | O |
| ATOM | 6463 | CB | GLN | D | 494 | −10.841 | −53.930 | 16.284 | 1.00 | 50.00 | C |
| ATOM | 6464 | CG | GLN | D | 494 | −11.535 | −55.044 | 15.502 | 1.00 | 50.00 | C |
| ATOM | 6465 | CD | GLN | D | 494 | −12.592 | −54.590 | 14.505 | 1.00 | 50.00 | C |
| ATOM | 6466 | OE1 | GLN | D | 494 | −12.877 | −53.409 | 14.312 | 1.00 | 50.00 | O |
| ATOM | 6467 | NE2 | GLN | D | 494 | −13.202 | −55.541 | 13.822 | 1.00 | 50.00 | N |
| ATOM | 6468 | N | VAL | D | 495 | −7.602 | −54.238 | 15.862 | 1.00 | 50.00 | N |
| ATOM | 6469 | CA | VAL | D | 495 | −6.346 | −54.753 | 15.310 | 1.00 | 50.00 | C |
| ATOM | 6470 | C | VAL | D | 495 | −5.544 | −55.528 | 16.351 | 1.00 | 50.00 | C |
| ATOM | 6471 | O | VAL | D | 495 | −5.332 | −56.720 | 16.135 | 1.00 | 50.00 | O |
| ATOM | 6472 | CB | VAL | D | 495 | −5.494 | −53.712 | 14.576 | 1.00 | 50.00 | C |
| ATOM | 6473 | CG1 | VAL | D | 495 | −4.230 | −54.307 | 13.954 | 1.00 | 50.00 | C |
| ATOM | 6474 | CG2 | VAL | D | 495 | −6.192 | −52.932 | 13.463 | 1.00 | 50.00 | C |
| ATOM | 6475 | N | ASN | D | 496 | −5.133 | −54.923 | 17.455 | 1.00 | 50.00 | N |
| ATOM | 6476 | CA | ASN | D | 496 | −4.460 | −55.591 | 18.570 | 1.00 | 50.00 | C |
| ATOM | 6477 | C | ASN | D | 496 | −5.142 | −56.828 | 19.147 | 1.00 | 50.00 | C |
| ATOM | 6478 | O | ASN | D | 496 | −4.444 | −57.801 | 19.426 | 1.00 | 50.00 | O |
| ATOM | 6479 | CB | ASN | D | 496 | −4.137 | −54.574 | 19.666 | 1.00 | 50.00 | C |
| ATOM | 6480 | CG | ASN | D | 496 | −3.115 | −53.500 | 19.317 | 1.00 | 50.00 | C |
| ATOM | 6481 | OD1 | ASN | D | 496 | −1.982 | −53.792 | 18.936 | 1.00 | 50.00 | O |
| ATOM | 6482 | ND2 | ASN | D | 496 | −3.468 | −52.233 | 19.438 | 1.00 | 50.00 | N |
| ATOM | 6483 | N | GLU | D | 497 | −6.461 | −56.852 | 19.260 | 1.00 | 50.00 | N |
| ATOM | 6484 | CA | GLU | D | 497 | −7.260 | −58.058 | 19.483 | 1.00 | 50.00 | C |
| ATOM | 6485 | C | GLU | D | 497 | −7.073 | −59.160 | 18.444 | 1.00 | 50.00 | C |
| ATOM | 6486 | O | GLU | D | 497 | −6.749 | −60.274 | 18.851 | 1.00 | 50.00 | O |
| ATOM | 6487 | CB | GLU | D | 497 | −8.727 | −57.663 | 19.662 | 1.00 | 50.00 | C |
| ATOM | 6488 | CG | GLU | D | 497 | −9.627 | −58.756 | 20.237 | 1.00 | 50.00 | C |
| ATOM | 6489 | CD | GLU | D | 497 | −11.087 | −58.348 | 20.368 | 1.00 | 50.00 | C |
| ATOM | 6490 | OE1 | GLU | D | 497 | −11.388 | −57.398 | 21.123 | 1.00 | 50.00 | O |
| ATOM | 6491 | OE2 | GLU | D | 497 | −11.951 | −58.983 | 19.726 | 1.00 | 50.00 | O |
| ATOM | 6492 | N | LYS | D | 498 | −7.219 | −58.897 | 17.155 | 1.00 | 50.00 | N |
| ATOM | 6493 | CA | LYS | D | 498 | −6.877 | −59.828 | 16.078 | 1.00 | 50.00 | C |
| ATOM | 6494 | C | LYS | D | 498 | −5.449 | −60.362 | 16.017 | 1.00 | 50.00 | C |
| ATOM | 6495 | O | LYS | D | 498 | −5.266 | −61.522 | 15.650 | 1.00 | 50.00 | O |
| ATOM | 6496 | CB | LYS | D | 498 | −7.292 | −59.256 | 14.721 | 1.00 | 50.00 | C |
| ATOM | 6497 | CG | LYS | D | 498 | −8.791 | −59.138 | 14.446 | 1.00 | 50.00 | C |
| ATOM | 6498 | CD | LYS | D | 498 | −9.507 | −60.482 | 14.329 | 1.00 | 50.00 | C |
| ATOM | 6499 | CE | LYS | D | 498 | −11.030 | −60.368 | 14.298 | 1.00 | 50.00 | C |
| ATOM | 6500 | NZ | LYS | D | 498 | −11.636 | −61.707 | 14.332 | 1.00 | 50.00 | N |
| ATOM | 6501 | N | ILE | D | 499 | −4.452 | −59.579 | 16.396 | 1.00 | 50.00 | N |
| ATOM | 6502 | CA | ILE | D | 499 | −3.068 | −60.014 | 16.594 | 1.00 | 50.00 | C |
| ATOM | 6503 | C | ILE | D | 499 | −2.930 | −60.965 | 17.780 | 1.00 | 50.00 | C |
| ATOM | 6504 | O | ILE | D | 499 | −2.314 | −62.016 | 17.611 | 1.00 | 50.00 | O |
| ATOM | 6505 | CB | ILE | D | 499 | −2.099 | −58.828 | 16.634 | 1.00 | 50.00 | C |
| ATOM | 6506 | CG1 | ILE | D | 499 | −2.247 | −57.893 | 15.431 | 1.00 | 50.00 | C |
| ATOM | 6507 | CG2 | ILE | D | 499 | −0.642 | −59.272 | 16.784 | 1.00 | 50.00 | C |
| ATOM | 6508 | CD1 | ILE | D | 499 | −1.477 | −56.578 | 15.495 | 1.00 | 50.00 | C |
| ATOM | 6509 | N | ASN | D | 500 | −3.464 | −60.654 | 18.951 | 1.00 | 50.00 | N |
| ATOM | 6510 | CA | ASN | D | 500 | −3.534 | −61.570 | 20.091 | 1.00 | 50.00 | C |
| ATOM | 6511 | C | ASN | D | 500 | −4.311 | −62.865 | 19.868 | 1.00 | 50.00 | C |
| ATOM | 6512 | O | ASN | D | 500 | −3.822 | −63.908 | 20.302 | 1.00 | 50.00 | O |
| ATOM | 6513 | CB | ASN | D | 500 | −4.011 | −60.820 | 21.336 | 1.00 | 50.00 | C |
| ATOM | 6514 | CG | ASN | D | 500 | −3.054 | −59.788 | 21.918 | 1.00 | 50.00 | C |
| ATOM | 6515 | OD1 | ASN | D | 500 | −1.855 | −60.023 | 22.058 | 1.00 | 50.00 | O |
| ATOM | 6516 | ND2 | ASN | D | 500 | −3.545 | −58.622 | 22.298 | 1.00 | 50.00 | N |
| ATOM | 6517 | N | GLN | D | 501 | −5.437 | −62.859 | 19.171 | 1.00 | 50.00 | N |
| ATOM | 6518 | CA | GLN | D | 501 | −6.065 | −64.044 | 18.583 | 1.00 | 50.00 | C |
| ATOM | 6519 | C | GLN | D | 501 | −5.178 | −64.882 | 17.667 | 1.00 | 50.00 | C |
| ATOM | 6520 | O | GLN | D | 501 | −5.133 | −66.097 | 17.857 | 1.00 | 50.00 | O |
| ATOM | 6521 | CB | GLN | D | 501 | −7.334 | −63.692 | 17.806 | 1.00 | 50.00 | C |
| ATOM | 6522 | CG | GLN | D | 501 | −8.535 | −63.124 | 18.561 | 1.00 | 50.00 | C |
| ATOM | 6523 | CD | GLN | D | 501 | −9.685 | −62.779 | 17.624 | 1.00 | 50.00 | C |
| ATOM | 6524 | OE1 | GLN | D | 501 | −10.092 | −63.572 | 16.777 | 1.00 | 50.00 | O |
| ATOM | 6525 | NE2 | GLN | D | 501 | −10.246 | −61.588 | 17.727 | 1.00 | 50.00 | N |
| ATOM | 6526 | N | SER | D | 502 | −4.478 | −64.291 | 16.712 | 1.00 | 50.00 | N |
| ATOM | 6527 | CA | SER | D | 502 | −3.447 | −64.948 | 15.906 | 1.00 | 50.00 | C |
| ATOM | 6528 | C | SER | D | 502 | −2.346 | −65.647 | 16.697 | 1.00 | 50.00 | C |
| ATOM | 6529 | O | SER | D | 502 | −2.138 | −66.840 | 16.479 | 1.00 | 50.00 | O |
| ATOM | 6530 | CB | SER | D | 502 | −2.891 | −63.954 | 14.886 | 1.00 | 50.00 | C |
| ATOM | 6531 | OG | SER | D | 502 | −1.988 | −64.588 | 13.992 | 1.00 | 50.00 | O |
| ATOM | 6532 | N | LEU | D | 503 | −1.705 | −64.973 | 17.638 | 1.00 | 50.00 | N |
| ATOM | 6533 | CA | LEU | D | 503 | −0.788 | −65.574 | 18.608 | 1.00 | 50.00 | C |
| ATOM | 6534 | C | LEU | D | 503 | −1.349 | −66.685 | 19.490 | 1.00 | 50.00 | C |
| ATOM | 6535 | O | LEU | D | 503 | −0.611 | −67.634 | 19.754 | 1.00 | 50.00 | O |
| ATOM | 6536 | CB | LEU | D | 503 | −0.123 | −64.480 | 19.445 | 1.00 | 50.00 | C |
| ATOM | 6537 | CG | LEU | D | 503 | 0.715 | −63.416 | 18.733 | 1.00 | 50.00 | C |
| ATOM | 6538 | CD1 | LEU | D | 503 | 1.173 | −62.329 | 19.704 | 1.00 | 50.00 | C |
| ATOM | 6539 | CD2 | LEU | D | 503 | 1.918 | −63.983 | 17.981 | 1.00 | 50.00 | C |
| ATOM | 6540 | N | ALA | D | 504 | −2.602 | −66.633 | 19.915 | 1.00 | 50.00 | N |
| ATOM | 6541 | CA | ALA | D | 504 | −3.312 | −67.742 | 20.555 | 1.00 | 50.00 | C |
| ATOM | 6542 | C | ALA | D | 504 | −3.590 | −68.969 | 19.693 | 1.00 | 50.00 | C |
| ATOM | 6543 | O | ALA | D | 504 | −3.308 | −70.072 | 20.159 | 1.00 | 50.00 | O |
| ATOM | 6544 | CB | ALA | D | 504 | −4.604 | −67.206 | 21.169 | 1.00 | 50.00 | C |
| ATOM | 6545 | N | PHE | D | 505 | −4.088 | −68.836 | 18.474 | 1.00 | 50.00 | N |
| ATOM | 6546 | CA | PHE | D | 505 | −4.161 | −69.924 | 17.496 | 1.00 | 50.00 | C |
| ATOM | 6547 | C | PHE | D | 505 | −2.851 | −70.613 | 17.131 | 1.00 | 50.00 | C |
| ATOM | 6548 | O | PHE | D | 505 | −2.832 | −71.836 | 16.996 | 1.00 | 50.00 | O |
| ATOM | 6549 | CB | PHE | D | 505 | −4.896 | −69.486 | 16.226 | 1.00 | 50.00 | C |
| ATOM | 6550 | CG | PHE | D | 505 | −6.398 | −69.313 | 16.309 | 1.00 | 50.00 | C |
| ATOM | 6551 | CD1 | PHE | D | 505 | −6.979 | −68.175 | 15.876 | 1.00 | 50.00 | C |
| ATOM | 6552 | CD2 | PHE | D | 505 | −7.175 | −70.311 | 16.781 | 1.00 | 50.00 | C |
| ATOM | 6553 | CE1 | PHE | D | 505 | −8.317 | −68.021 | 15.944 | 1.00 | 50.00 | C |
| ATOM | 6554 | CE2 | PHE | D | 505 | −8.512 | −70.158 | 16.867 | 1.00 | 50.00 | C |
| ATOM | 6555 | CZ | PHE | D | 505 | −9.082 | −69.011 | 16.446 | 1.00 | 50.00 | C |
| ATOM | 6556 | N | ILE | D | 506 | −1.763 | −69.869 | 17.017 | 1.00 | 50.00 | N |
| ATOM | 6557 | CA | ILE | D | 506 | −0.402 | −70.399 | 16.932 | 1.00 | 50.00 | C |
| ATOM | 6558 | C | ILE | D | 506 | 0.050 | −71.102 | 18.208 | 1.00 | 50.00 | C |
| ATOM | 6559 | O | ILE | D | 506 | 0.598 | −72.195 | 18.082 | 1.00 | 50.00 | O |
| ATOM | 6560 | CB | ILE | D | 506 | 0.560 | −69.315 | 16.438 | 1.00 | 50.00 | C |
| ATOM | 6561 | CG1 | ILE | D | 506 | 0.187 | −68.729 | 15.073 | 1.00 | 50.00 | C |
| ATOM | 6562 | CG2 | ILE | D | 506 | 2.018 | −69.770 | 16.515 | 1.00 | 50.00 | C |
| ATOM | 6563 | CD1 | ILE | D | 506 | 0.276 | −69.658 | 13.865 | 1.00 | 50.00 | C |
| ATOM | 6564 | N | ARG | D | 507 | −0.109 | −70.527 | 19.390 | 1.00 | 50.00 | N |
| ATOM | 6565 | CA | ARG | D | 507 | 0.127 | −71.207 | 20.665 | 1.00 | 50.00 | C |
| ATOM | 6566 | C | ARG | D | 507 | −0.506 | −72.589 | 20.802 | 1.00 | 50.00 | C |
| ATOM | 6567 | O | ARG | D | 507 | 0.178 | −73.512 | 21.241 | 1.00 | 50.00 | O |
| ATOM | 6568 | CB | ARG | D | 507 | −0.248 | −70.265 | 21.810 | 1.00 | 50.00 | C |
| ATOM | 6569 | CG | ARG | D | 507 | 0.328 | −70.680 | 23.163 | 1.00 | 50.00 | C |
| ATOM | 6570 | CD | ARG | D | 507 | −0.219 | −69.925 | 24.373 | 1.00 | 50.00 | C |
| ATOM | 6571 | NE | ARG | D | 507 | 0.577 | −70.102 | 25.588 | 1.00 | 50.00 | N |
| ATOM | 6572 | CZ | ARG | D | 507 | 0.782 | −71.242 | 26.228 | 1.00 | 50.00 | C |
| ATOM | 6573 | NH1 | ARG | D | 507 | 0.224 | −72.395 | 25.905 | 1.00 | 50.00 | N |
| ATOM | 6574 | NH2 | ARG | D | 507 | 1.614 | −71.237 | 27.252 | 1.00 | 50.00 | N |
| ATOM | 6575 | N | LYS | D | 508 | −1.747 | −72.760 | 20.374 | 1.00 | 50.00 | N |
| ATOM | 6576 | CA | LYS | D | 508 | −2.408 | −74.058 | 20.237 | 1.00 | 50.00 | C |
| ATOM | 6577 | C | LYS | D | 508 | −1.856 | −74.989 | 19.162 | 1.00 | 50.00 | C |
| ATOM | 6578 | O | LYS | D | 508 | −1.712 | −76.177 | 19.450 | 1.00 | 50.00 | O |
| ATOM | 6579 | CB | LYS | D | 508 | −3.912 | −73.860 | 20.044 | 1.00 | 50.00 | C |
| ATOM | 6580 | CG | LYS | D | 508 | −4.630 | −73.179 | 21.208 | 1.00 | 50.00 | C |
| ATOM | 6581 | CD | LYS | D | 508 | −6.150 | −73.237 | 21.077 | 1.00 | 50.00 | C |
| ATOM | 6582 | CE | LYS | D | 508 | −6.848 | −72.618 | 22.286 | 1.00 | 50.00 | C |
| ATOM | 6583 | NZ | LYS | D | 508 | −8.307 | −72.769 | 22.175 | 1.00 | 50.00 | N |
| ATOM | 6584 | N | SER | D | 509 | −1.526 | −74.520 | 17.969 | 1.00 | 50.00 | N |
| ATOM | 6585 | CA | SER | D | 509 | −0.790 | −75.294 | 16.969 | 1.00 | 50.00 | C |
| ATOM | 6586 | C | SER | D | 509 | 0.564 | −75.833 | 17.419 | 1.00 | 50.00 | C |
| ATOM | 6587 | O | SER | D | 509 | 0.788 | −77.035 | 17.289 | 1.00 | 50.00 | O |
| ATOM | 6588 | CB | SER | D | 509 | −0.696 | −74.509 | 15.661 | 1.00 | 50.00 | C |
| ATOM | 6589 | OG | SER | D | 509 | −0.396 | −75.399 | 14.596 | 1.00 | 50.00 | O |
| ATOM | 6590 | N | ASP | D | 510 | 1.419 | −75.012 | 18.008 | 1.00 | 50.00 | N |
| ATOM | 6591 | CA | ASP | D | 510 | 2.637 | −75.427 | 18.706 | 1.00 | 50.00 | C |
| ATOM | 6592 | C | ASP | D | 510 | 2.444 | −76.447 | 19.824 | 1.00 | 50.00 | C |
| ATOM | 6593 | O | ASP | D | 510 | 3.134 | −77.465 | 19.787 | 1.00 | 50.00 | O |
| ATOM | 6594 | CB | ASP | D | 510 | 3.418 | −74.207 | 19.200 | 1.00 | 50.00 | C |
| ATOM | 6595 | CG | ASP | D | 510 | 3.961 | −73.241 | 18.157 | 1.00 | 50.00 | C |
| ATOM | 6596 | OD1 | ASP | D | 510 | 4.665 | −73.679 | 17.222 | 1.00 | 50.00 | O |
| ATOM | 6597 | OD2 | ASP | D | 510 | 3.706 | −72.023 | 18.272 | 1.00 | 50.00 | O |
| ATOM | 6598 | N | GLU | D | 511 | 1.520 | −76.256 | 20.754 | 1.00 | 50.00 | N |
| ATOM | 6599 | CA | GLU | D | 511 | 1.089 | −77.256 | 21.734 | 1.00 | 50.00 | C |
| ATOM | 6600 | C | GLU | D | 511 | 0.806 | −78.655 | 21.196 | 1.00 | 50.00 | C |
| ATOM | 6601 | O | GLU | D | 511 | 1.453 | −79.602 | 21.645 | 1.00 | 50.00 | O |
| ATOM | 6602 | CB | GLU | D | 511 | −0.129 | −76.755 | 22.513 | 1.00 | 50.00 | C |
| ATOM | 6603 | CG | GLU | D | 511 | 0.075 | −75.699 | 23.599 | 1.00 | 50.00 | C |
| ATOM | 6604 | CD | GLU | D | 511 | −1.255 | −75.167 | 24.112 | 1.00 | 50.00 | C |
| ATOM | 6605 | OE1 | GLU | D | 511 | −2.022 | −75.939 | 24.728 | 1.00 | 50.00 | O |
| ATOM | 6606 | OE2 | GLU | D | 511 | −1.559 | −73.975 | 23.890 | 1.00 | 50.00 | O |
| ATOM | 6607 | N | LEU | D | 512 | −0.098 | −78.796 | 20.239 | 1.00 | 50.00 | N |
| ATOM | 6608 | CA | LEU | D | 512 | −0.389 | −80.044 | 19.530 | 1.00 | 50.00 | C |
| ATOM | 6609 | C | LEU | D | 512 | 0.790 | −80.772 | 18.893 | 1.00 | 50.00 | C |
| ATOM | 6610 | O | LEU | D | 512 | 0.921 | −81.984 | 19.058 | 1.00 | 50.00 | O |
| ATOM | 6611 | CB | LEU | D | 512 | −1.466 | −79.796 | 18.471 | 1.00 | 50.00 | C |
| ATOM | 6612 | CG | LEU | D | 512 | −2.872 | −79.401 | 18.925 | 1.00 | 50.00 | C |
| ATOM | 6613 | CD1 | LEU | D | 512 | −3.737 | −79.032 | 17.720 | 1.00 | 50.00 | C |
| ATOM | 6614 | CD2 | LEU | D | 512 | −3.556 | −80.483 | 19.758 | 1.00 | 50.00 | C |
| ATOM | 6615 | N | LEU | D | 513 | 1.648 | −80.054 | 18.189 | 1.00 | 50.00 | N |
| ATOM | 6616 | CA | LEU | D | 513 | 2.899 | −80.558 | 17.620 | 1.00 | 50.00 | C |
| ATOM | 6617 | C | LEU | D | 513 | 3.913 | −81.105 | 18.619 | 1.00 | 50.00 | C |
| ATOM | 6618 | O | LEU | D | 513 | 4.433 | −82.198 | 18.396 | 1.00 | 50.00 | O |
| ATOM | 6619 | CB | LEU | D | 513 | 3.493 | −79.443 | 16.758 | 1.00 | 50.00 | C |
| ATOM | 6620 | CG | LEU | D | 513 | 2.667 | −78.987 | 15.555 | 1.00 | 50.00 | C |
| ATOM | 6621 | CD1 | LEU | D | 513 | 3.213 | −77.661 | 15.035 | 1.00 | 50.00 | C |
| ATOM | 6622 | CD2 | LEU | D | 513 | 2.643 | −79.971 | 14.388 | 1.00 | 50.00 | C |
| ATOM | 6623 | N | HIS | D | 514 | 4.159 | −80.417 | 19.723 | 1.00 | 50.00 | N |
| ATOM | 6624 | CA | HIS | D | 514 | 4.911 | −80.932 | 20.869 | 1.00 | 50.00 | C |
| ATOM | 6625 | C | HIS | D | 514 | 4.316 | −82.107 | 21.640 | 1.00 | 50.00 | C |
| ATOM | 6626 | O | HIS | D | 514 | 5.079 | −82.772 | 22.340 | 1.00 | 50.00 | O |
| ATOM | 6627 | CB | HIS | D | 514 | 5.271 | −79.775 | 21.802 | 1.00 | 50.00 | C |
| ATOM | 6628 | CG | HIS | D | 514 | 6.147 | −78.672 | 21.246 | 1.00 | 50.00 | C |
| ATOM | 6629 | ND1 | HIS | D | 514 | 7.343 | −78.889 | 20.589 | 1.00 | 50.00 | N |
| ATOM | 6630 | CD2 | HIS | D | 514 | 5.820 | −77.319 | 21.251 | 1.00 | 50.00 | C |
| ATOM | 6631 | CE1 | HIS | D | 514 | 7.623 | −77.593 | 20.209 | 1.00 | 50.00 | C |
| ATOM | 6632 | NE2 | HIS | D | 514 | 6.761 | −76.578 | 20.565 | 1.00 | 50.00 | N |
| ATOM | 6633 | N | ASN | D | 515 | 3.037 | −82.430 | 21.520 | 1.00 | 50.00 | N |
| ATOM | 6634 | CA | ASN | D | 515 | 2.415 | −83.623 | 22.098 | 1.00 | 50.00 | C |
| ATOM | 6635 | C | ASN | D | 515 | 2.890 | −84.872 | 21.367 | 1.00 | 50.00 | C |
| ATOM | 6636 | O | ASN | D | 515 | 2.284 | −85.421 | 20.447 | 1.00 | 50.00 | O |
| ATOM | 6637 | CB | ASN | D | 515 | 0.896 | −83.442 | 22.101 | 1.00 | 50.00 | C |
| ATOM | 6638 | CG | ASN | D | 515 | 0.095 | −84.503 | 22.843 | 1.00 | 50.00 | C |
| ATOM | 6639 | OD1 | ASN | D | 515 | −0.113 | −85.609 | 22.346 | 1.00 | 50.00 | O |
| ATOM | 6640 | ND2 | ASN | D | 515 | −0.391 | −84.213 | 24.036 | 1.00 | 50.00 | N |
| ATOM | 6641 | N | THR | E | 29 | 19.566 | 51.742 | −6.326 | 1.00 | 50.00 | N |
| ATOM | 6642 | CA | THR | E | 29 | 20.131 | 52.868 | −5.565 | 1.00 | 50.00 | C |
| ATOM | 6643 | C | THR | E | 29 | 21.650 | 52.824 | −5.452 | 1.00 | 50.00 | C |
| ATOM | 6644 | O | THR | E | 29 | 22.239 | 53.901 | −5.541 | 1.00 | 50.00 | O |
| ATOM | 6645 | CB | THR | E | 29 | 19.424 | 53.066 | −4.222 | 1.00 | 50.00 | C |
| ATOM | 6646 | OG1 | THR | E | 29 | 18.080 | 53.464 | −4.449 | 1.00 | 50.00 | O |
| ATOM | 6647 | CG2 | THR | E | 29 | 20.023 | 54.117 | −3.288 | 1.00 | 50.00 | C |
| ATOM | 6648 | N | GLU | E | 30 | 22.273 | 51.690 | −5.170 | 1.00 | 50.00 | N |
| ATOM | 6649 | CA | GLU | E | 30 | 23.679 | 51.591 | −4.774 | 1.00 | 50.00 | C |
| ATOM | 6650 | C | GLU | E | 30 | 23.867 | 51.890 | −3.291 | 1.00 | 50.00 | C |
| ATOM | 6651 | O | GLU | E | 30 | 22.927 | 52.067 | −2.515 | 1.00 | 50.00 | O |
| ATOM | 6652 | CB | GLU | E | 30 | 24.673 | 52.256 | −5.732 | 1.00 | 50.00 | C |
| ATOM | 6653 | CG | GLU | E | 30 | 24.669 | 51.764 | −7.178 | 1.00 | 50.00 | C |
| ATOM | 6654 | CD | GLU | E | 30 | 25.415 | 50.448 | −7.340 | 1.00 | 50.00 | C |
| ATOM | 6655 | OE1 | GLU | E | 30 | 24.788 | 49.375 | −7.209 | 1.00 | 50.00 | O |
| ATOM | 6656 | OE2 | GLU | E | 30 | 26.643 | 50.480 | −7.573 | 1.00 | 50.00 | O |
| ATOM | 6657 | N | GLU | E | 31 | 25.124 | 51.859 | −2.883 | 1.00 | 50.00 | N |
| ATOM | 6658 | CA | GLU | E | 31 | 25.569 | 52.012 | −1.499 | 1.00 | 50.00 | C |
| ATOM | 6659 | C | GLU | E | 31 | 26.687 | 53.033 | −1.330 | 1.00 | 50.00 | C |
| ATOM | 6660 | O | GLU | E | 31 | 27.440 | 52.946 | −0.361 | 1.00 | 50.00 | O |
| ATOM | 6661 | CB | GLU | E | 31 | 26.040 | 50.630 | −1.044 | 1.00 | 50.00 | C |
| ATOM | 6662 | CG | GLU | E | 31 | 25.042 | 49.473 | −1.029 | 1.00 | 50.00 | C |
| ATOM | 6663 | CD | GLU | E | 31 | 23.814 | 49.574 | −0.138 | 1.00 | 50.00 | C |
| ATOM | 6664 | OE1 | GLU | E | 31 | 23.157 | 50.636 | −0.070 | 1.00 | 50.00 | O |
| ATOM | 6665 | OE2 | GLU | E | 31 | 23.489 | 48.558 | 0.514 | 1.00 | 50.00 | O |
| ATOM | 6666 | N | PHE | E | 32 | 26.763 | 54.074 | −2.146 | 1.00 | 50.00 | N |
| ATOM | 6667 | CA | PHE | E | 32 | 27.447 | 55.300 | −1.740 | 1.00 | 50.00 | C |
| ATOM | 6668 | C | PHE | E | 32 | 26.712 | 56.014 | −0.608 | 1.00 | 50.00 | C |
| ATOM | 6669 | O | PHE | E | 32 | 27.362 | 56.449 | 0.342 | 1.00 | 50.00 | O |
| ATOM | 6670 | CB | PHE | E | 32 | 27.882 | 56.147 | −2.938 | 1.00 | 50.00 | C |
| ATOM | 6671 | CG | PHE | E | 32 | 29.115 | 56.968 | −2.621 | 1.00 | 50.00 | C |
| ATOM | 6672 | CD1 | PHE | E | 32 | 30.339 | 56.792 | −3.161 | 1.00 | 50.00 | C |
| ATOM | 6673 | CD2 | PHE | E | 32 | 28.975 | 57.879 | −1.638 | 1.00 | 50.00 | C |
| ATOM | 6674 | CE1 | PHE | E | 32 | 31.370 | 57.580 | −2.791 | 1.00 | 50.00 | C |
| ATOM | 6675 | CE2 | PHE | E | 32 | 30.014 | 58.616 | −1.201 | 1.00 | 50.00 | C |
| ATOM | 6676 | CZ | PHE | E | 32 | 31.198 | 58.515 | −1.834 | 1.00 | 50.00 | C |
| ATOM | 6677 | N | TYR | E | 33 | 25.391 | 56.067 | −0.615 | 1.00 | 50.00 | N |
| ATOM | 6678 | CA | TYR | E | 33 | 24.587 | 56.385 | 0.565 | 1.00 | 50.00 | C |
| ATOM | 6679 | C | TYR | E | 33 | 24.791 | 55.674 | 1.900 | 1.00 | 50.00 | C |
| ATOM | 6680 | O | TYR | E | 33 | 24.299 | 56.147 | 2.925 | 1.00 | 50.00 | O |
| ATOM | 6681 | CB | TYR | E | 33 | 23.145 | 56.392 | 0.053 | 1.00 | 50.00 | C |
| ATOM | 6682 | CG | TYR | E | 33 | 22.138 | 56.988 | 1.010 | 1.00 | 50.00 | C |
| ATOM | 6683 | CD1 | TYR | E | 33 | 22.490 | 58.112 | 1.665 | 1.00 | 50.00 | C |
| ATOM | 6684 | CD2 | TYR | E | 33 | 21.004 | 56.347 | 1.361 | 1.00 | 50.00 | C |
| ATOM | 6685 | CE1 | TYR | E | 33 | 21.847 | 58.465 | 2.793 | 1.00 | 50.00 | C |
| ATOM | 6686 | CE2 | TYR | E | 33 | 20.228 | 56.849 | 2.342 | 1.00 | 50.00 | C |
| ATOM | 6687 | CZ | TYR | E | 33 | 20.684 | 57.864 | 3.106 | 1.00 | 50.00 | C |
| ATOM | 6688 | OH | TYR | E | 33 | 19.955 | 58.449 | 4.108 | 1.00 | 50.00 | O |
| ATOM | 6689 | N | GLN | E | 34 | 25.573 | 54.611 | 1.962 | 1.00 | 50.00 | N |
| ATOM | 6690 | CA | GLN | E | 34 | 26.140 | 54.081 | 3.203 | 1.00 | 50.00 | C |
| ATOM | 6691 | C | GLN | E | 34 | 27.073 | 55.002 | 3.981 | 1.00 | 50.00 | C |
| ATOM | 6692 | O | GLN | E | 34 | 26.907 | 55.117 | 5.194 | 1.00 | 50.00 | O |
| ATOM | 6693 | CB | GLN | E | 34 | 26.866 | 52.772 | 2.896 | 1.00 | 50.00 | C |
| ATOM | 6694 | CG | GLN | E | 34 | 25.988 | 51.627 | 2.395 | 1.00 | 50.00 | C |
| ATOM | 6695 | CD | GLN | E | 34 | 25.031 | 51.024 | 3.411 | 1.00 | 50.00 | C |
| ATOM | 6696 | OE1 | GLN | E | 34 | 25.078 | 51.246 | 4.620 | 1.00 | 50.00 | O |
| ATOM | 6697 | NE2 | GLN | E | 34 | 24.118 | 50.226 | 2.891 | 1.00 | 50.00 | N |
| ATOM | 6698 | N | SER | E | 35 | 28.009 | 55.653 | 3.307 | 1.00 | 50.00 | N |
| ATOM | 6699 | CA | SER | E | 35 | 28.783 | 56.782 | 3.827 | 1.00 | 50.00 | C |
| ATOM | 6700 | C | SER | E | 35 | 28.050 | 58.050 | 4.246 | 1.00 | 50.00 | C |
| ATOM | 6701 | O | SER | E | 35 | 28.630 | 58.885 | 4.940 | 1.00 | 50.00 | O |
| ATOM | 6702 | CB | SER | E | 35 | 29.762 | 57.251 | 2.751 | 1.00 | 50.00 | C |
| ATOM | 6703 | OG | SER | E | 35 | 30.661 | 56.223 | 2.364 | 1.00 | 50.00 | O |
| ATOM | 6704 | N | THR | E | 36 | 26.820 | 58.233 | 3.800 | 1.00 | 50.00 | N |
| ATOM | 6705 | CA | THR | E | 36 | 25.899 | 59.227 | 4.349 | 1.00 | 50.00 | C |
| ATOM | 6706 | C | THR | E | 36 | 24.979 | 58.678 | 5.437 | 1.00 | 50.00 | C |
| ATOM | 6707 | O | THR | E | 36 | 24.333 | 59.506 | 6.077 | 1.00 | 50.00 | O |
| ATOM | 6708 | CB | THR | E | 36 | 25.239 | 59.844 | 3.115 | 1.00 | 50.00 | C |
| ATOM | 6709 | OG1 | THR | E | 36 | 26.162 | 60.412 | 2.197 | 1.00 | 50.00 | O |
| ATOM | 6710 | CG2 | THR | E | 36 | 24.246 | 60.956 | 3.436 | 1.00 | 50.00 | C |
| ATOM | 6711 | N | CYS | E | 37 | 24.961 | 57.397 | 5.776 | 1.00 | 50.00 | N |
| ATOM | 6712 | CA | CYS | E | 37 | 24.471 | 56.819 | 7.029 | 1.00 | 50.00 | C |
| ATOM | 6713 | C | CYS | E | 37 | 23.147 | 56.064 | 7.008 | 1.00 | 50.00 | C |
| ATOM | 6714 | O | CYS | E | 37 | 22.413 | 55.987 | 7.992 | 1.00 | 50.00 | O |
| ATOM | 6715 | CB | CYS | E | 37 | 24.594 | 57.735 | 8.243 | 1.00 | 50.00 | C |
| ATOM | 6716 | SG | CYS | E | 37 | 26.133 | 58.669 | 8.165 | 1.00 | 50.00 | S |
| ATOM | 6717 | N | SER | E | 38 | 22.903 | 55.417 | 5.882 | 1.00 | 50.00 | N |
| ATOM | 6718 | CA | SER | E | 38 | 21.734 | 54.568 | 5.675 | 1.00 | 50.00 | C |
| ATOM | 6719 | C | SER | E | 38 | 22.129 | 53.323 | 4.889 | 1.00 | 50.00 | C |
| ATOM | 6720 | O | SER | E | 38 | 22.891 | 53.269 | 3.943 | 1.00 | 50.00 | O |
| ATOM | 6721 | CB | SER | E | 38 | 20.716 | 55.263 | 4.782 | 1.00 | 50.00 | C |
| ATOM | 6722 | OG | SER | E | 38 | 19.994 | 56.253 | 5.499 | 1.00 | 50.00 | O |
| ATOM | 6723 | N | ALA | E | 39 | 21.367 | 52.344 | 5.362 | 1.00 | 50.00 | N |
| ATOM | 6724 | CA | ALA | E | 39 | 21.567 | 50.932 | 5.072 | 1.00 | 50.00 | C |
| ATOM | 6725 | C | ALA | E | 39 | 20.366 | 50.522 | 4.232 | 1.00 | 50.00 | C |
| ATOM | 6726 | O | ALA | E | 39 | 19.221 | 50.614 | 4.673 | 1.00 | 50.00 | O |
| ATOM | 6727 | CB | ALA | E | 39 | 21.787 | 50.310 | 6.449 | 1.00 | 50.00 | C |
| ATOM | 6728 | N | VAL | E | 40 | 20.638 | 50.051 | 3.026 | 1.00 | 50.00 | N |
| ATOM | 6729 | CA | VAL | E | 40 | 19.652 | 49.324 | 2.227 | 1.00 | 50.00 | C |
| ATOM | 6730 | C | VAL | E | 40 | 19.564 | 47.955 | 2.890 | 1.00 | 50.00 | C |
| ATOM | 6731 | O | VAL | E | 40 | 20.447 | 47.111 | 2.734 | 1.00 | 50.00 | O |
| ATOM | 6732 | CB | VAL | E | 40 | 19.973 | 49.293 | 0.730 | 1.00 | 50.00 | C |
| ATOM | 6733 | CG1 | VAL | E | 40 | 19.139 | 48.301 | −0.081 | 1.00 | 50.00 | C |
| ATOM | 6734 | CG2 | VAL | E | 40 | 19.839 | 50.665 | 0.070 | 1.00 | 50.00 | C |
| ATOM | 6735 | N | SER | E | 41 | 18.483 | 47.754 | 3.625 | 1.00 | 50.00 | N |
| ATOM | 6736 | CA | SER | E | 41 | 18.100 | 46.428 | 4.111 | 1.00 | 50.00 | C |
| ATOM | 6737 | C | SER | E | 41 | 17.773 | 45.487 | 2.958 | 1.00 | 50.00 | C |
| ATOM | 6738 | O | SER | E | 41 | 18.374 | 44.414 | 2.915 | 1.00 | 50.00 | O |
| ATOM | 6739 | CB | SER | E | 41 | 16.962 | 46.513 | 5.126 | 1.00 | 50.00 | C |
| ATOM | 6740 | OG | SER | E | 41 | 17.308 | 47.289 | 6.265 | 1.00 | 50.00 | O |
| ATOM | 6741 | N | LYS | E | 42 | 16.865 | 45.839 | 2.060 | 1.00 | 50.00 | N |
| ATOM | 6742 | CA | LYS | E | 42 | 16.329 | 44.916 | 1.060 | 1.00 | 50.00 | C |
| ATOM | 6743 | C | LYS | E | 42 | 15.927 | 45.652 | −0.211 | 1.00 | 50.00 | C |
| ATOM | 6744 | O | LYS | E | 42 | 15.347 | 46.736 | −0.174 | 1.00 | 50.00 | O |
| ATOM | 6745 | CB | LYS | E | 42 | 15.153 | 44.108 | 1.612 | 1.00 | 50.00 | C |
| ATOM | 6746 | CG | LYS | E | 42 | 15.591 | 43.119 | 2.691 | 1.00 | 50.00 | C |
| ATOM | 6747 | CD | LYS | E | 42 | 14.532 | 42.157 | 3.220 | 1.00 | 50.00 | C |
| ATOM | 6748 | CE | LYS | E | 42 | 15.156 | 41.306 | 4.324 | 1.00 | 50.00 | C |
| ATOM | 6749 | NZ | LYS | E | 42 | 14.170 | 40.360 | 4.866 | 1.00 | 50.00 | N |
| ATOM | 6750 | N | GLY | E | 43 | 16.193 | 45.019 | −1.340 | 1.00 | 50.00 | N |
| ATOM | 6751 | CA | GLY | E | 43 | 15.619 | 45.397 | −2.630 | 1.00 | 50.00 | C |
| ATOM | 6752 | C | GLY | E | 43 | 14.718 | 44.270 | −3.114 | 1.00 | 50.00 | C |
| ATOM | 6753 | O | GLY | E | 43 | 15.019 | 43.080 | −3.033 | 1.00 | 50.00 | O |
| ATOM | 6754 | N | TYR | E | 44 | 13.578 | 44.709 | −3.614 | 1.00 | 50.00 | N |
| ATOM | 6755 | CA | TYR | E | 44 | 12.479 | 43.842 | −4.032 | 1.00 | 50.00 | C |
| ATOM | 6756 | C | TYR | E | 44 | 11.901 | 44.386 | −5.334 | 1.00 | 50.00 | C |
| ATOM | 6757 | O | TYR | E | 44 | 11.956 | 45.577 | −5.627 | 1.00 | 50.00 | O |
| ATOM | 6758 | CB | TYR | E | 44 | 11.490 | 43.697 | −2.872 | 1.00 | 50.00 | C |
| ATOM | 6759 | CG | TYR | E | 44 | 10.306 | 42.832 | −3.241 | 1.00 | 50.00 | C |
| ATOM | 6760 | CD1 | TYR | E | 44 | 10.609 | 41.610 | −3.727 | 1.00 | 50.00 | C |
| ATOM | 6761 | CD2 | TYR | E | 44 | 9.213 | 43.479 | −3.696 | 1.00 | 50.00 | C |
| ATOM | 6762 | CE1 | TYR | E | 44 | 9.975 | 41.182 | −4.836 | 1.00 | 50.00 | C |
| ATOM | 6763 | CE2 | TYR | E | 44 | 8.432 | 42.901 | −4.630 | 1.00 | 50.00 | C |
| ATOM | 6764 | CZ | TYR | E | 44 | 8.820 | 41.757 | −5.224 | 1.00 | 50.00 | C |
| ATOM | 6765 | OH | TYR | E | 44 | 7.968 | 41.138 | −6.099 | 1.00 | 50.00 | O |
| ATOM | 6766 | N | LEU | E | 45 | 11.274 | 43.516 | −6.105 | 1.00 | 50.00 | N |
| ATOM | 6767 | CA | LEU | E | 45 | 10.434 | 43.767 | −7.276 | 1.00 | 50.00 | C |
| ATOM | 6768 | C | LEU | E | 45 | 9.084 | 44.461 | −7.100 | 1.00 | 50.00 | C |
| ATOM | 6769 | O | LEU | E | 45 | 9.033 | 45.290 | −6.193 | 1.00 | 50.00 | O |
| ATOM | 6770 | CB | LEU | E | 45 | 10.612 | 42.443 | −8.021 | 1.00 | 50.00 | C |
| ATOM | 6771 | CG | LEU | E | 45 | 11.957 | 42.082 | −8.652 | 1.00 | 50.00 | C |
| ATOM | 6772 | CD1 | LEU | E | 45 | 13.174 | 41.857 | −7.755 | 1.00 | 50.00 | C |
| ATOM | 6773 | CD2 | LEU | E | 45 | 12.002 | 41.056 | −9.779 | 1.00 | 50.00 | C |
| ATOM | 6774 | N | SER | E | 46 | 8.015 | 44.235 | −7.850 | 1.00 | 50.00 | N |
| ATOM | 6775 | CA | SER | E | 46 | 6.651 | 44.663 | −7.526 | 1.00 | 50.00 | C |
| ATOM | 6776 | C | SER | E | 46 | 5.625 | 44.184 | −8.544 | 1.00 | 50.00 | C |
| ATOM | 6777 | O | SER | E | 46 | 5.932 | 44.087 | −9.732 | 1.00 | 50.00 | O |
| ATOM | 6778 | CB | SER | E | 46 | 6.367 | 46.165 | −7.477 | 1.00 | 50.00 | C |
| ATOM | 6779 | OG | SER | E | 46 | 6.923 | 46.754 | −6.314 | 1.00 | 50.00 | O |
| ATOM | 6780 | N | ALA | E | 47 | 4.401 | 43.934 | −8.101 | 1.00 | 50.00 | N |
| ATOM | 6781 | CA | ALA | E | 47 | 3.367 | 43.325 | −8.938 | 1.00 | 50.00 | C |
| ATOM | 6782 | C | ALA | E | 47 | 1.986 | 43.137 | −8.320 | 1.00 | 50.00 | C |
| ATOM | 6783 | O | ALA | E | 47 | 1.827 | 43.071 | −7.102 | 1.00 | 50.00 | O |
| ATOM | 6784 | CB | ALA | E | 47 | 3.925 | 41.946 | −9.266 | 1.00 | 50.00 | C |
| ATOM | 6785 | N | LEU | E | 48 | 0.984 | 43.058 | −9.181 | 1.00 | 50.00 | N |
| ATOM | 6786 | CA | LEU | E | 48 | −0.436 | 43.190 | −8.851 | 1.00 | 50.00 | C |
| ATOM | 6787 | C | LEU | E | 48 | −1.158 | 41.865 | −9.057 | 1.00 | 50.00 | C |
| ATOM | 6788 | O | LEU | E | 48 | −0.856 | 41.101 | −9.973 | 1.00 | 50.00 | O |
| ATOM | 6789 | CB | LEU | E | 48 | −1.146 | 44.305 | −9.622 | 1.00 | 50.00 | C |
| ATOM | 6790 | CG | LEU | E | 48 | −0.834 | 45.752 | −9.235 | 1.00 | 50.00 | C |
| ATOM | 6791 | CD1 | LEU | E | 48 | 0.655 | 46.082 | −9.171 | 1.00 | 50.00 | C |
| ATOM | 6792 | CD2 | LEU | E | 48 | −1.488 | 46.811 | −10.120 | 1.00 | 50.00 | C |
| ATOM | 6793 | N | ARG | E | 49 | −2.113 | 41.588 | −8.184 | 1.00 | 50.00 | N |
| ATOM | 6794 | CA | ARG | E | 49 | −2.693 | 40.253 | −8.036 | 1.00 | 50.00 | C |
| ATOM | 6795 | C | ARG | E | 49 | −4.172 | 40.303 | −7.694 | 1.00 | 50.00 | C |
| ATOM | 6796 | O | ARG | E | 49 | −4.716 | 41.333 | −7.299 | 1.00 | 50.00 | O |
| ATOM | 6797 | CB | ARG | E | 49 | −2.066 | 39.442 | −6.903 | 1.00 | 50.00 | C |
| ATOM | 6798 | CG | ARG | E | 49 | −0.568 | 39.286 | −7.129 | 1.00 | 50.00 | C |
| ATOM | 6799 | CD | ARG | E | 49 | −0.020 | 38.123 | −6.315 | 1.00 | 50.00 | C |
| ATOM | 6800 | NE | ARG | E | 49 | 1.427 | 38.322 | −6.318 | 1.00 | 50.00 | N |
| ATOM | 6801 | CZ | ARG | E | 49 | 2.324 | 37.544 | −5.744 | 1.00 | 50.00 | C |
| ATOM | 6802 | NH1 | ARG | E | 49 | 1.915 | 36.441 | −5.157 | 1.00 | 50.00 | N |
| ATOM | 6803 | NH2 | ARG | E | 49 | 3.601 | 37.869 | −5.677 | 1.00 | 50.00 | N |
| ATOM | 6804 | N | THR | E | 50 | −4.772 | 39.130 | −7.813 | 1.00 | 50.00 | N |
| ATOM | 6805 | CA | THR | E | 50 | −6.188 | 38.931 | −7.521 | 1.00 | 50.00 | C |
| ATOM | 6806 | C | THR | E | 50 | −6.406 | 37.460 | −7.195 | 1.00 | 50.00 | C |
| ATOM | 6807 | O | THR | E | 50 | −6.041 | 36.618 | −8.014 | 1.00 | 50.00 | O |
| ATOM | 6808 | CB | THR | E | 50 | −6.940 | 39.240 | −8.814 | 1.00 | 50.00 | C |
| ATOM | 6809 | OG1 | THR | E | 50 | −6.445 | 40.357 | −9.538 | 1.00 | 50.00 | O |
| ATOM | 6810 | CG2 | THR | E | 50 | −8.449 | 39.345 | −8.632 | 1.00 | 50.00 | C |
| ATOM | 6811 | N | GLY | E | 51 | −7.149 | 37.170 | −6.140 | 1.00 | 50.00 | N |
| ATOM | 6812 | CA | GLY | E | 51 | −7.683 | 35.829 | −5.903 | 1.00 | 50.00 | C |
| ATOM | 6813 | C | GLY | E | 51 | −8.953 | 35.498 | −6.674 | 1.00 | 50.00 | C |
| ATOM | 6814 | O | GLY | E | 51 | −10.035 | 35.405 | −6.097 | 1.00 | 50.00 | O |
| ATOM | 6815 | N | TRP | E | 52 | −8.791 | 35.310 | −7.973 | 1.00 | 50.00 | N |
| ATOM | 6816 | CA | TRP | E | 52 | −9.862 | 35.120 | −8.953 | 1.00 | 50.00 | C |
| ATOM | 6817 | C | TRP | E | 52 | −9.966 | 33.699 | −9.492 | 1.00 | 50.00 | C |
| ATOM | 6818 | O | TRP | E | 52 | −11.085 | 33.217 | −9.667 | 1.00 | 50.00 | O |
| ATOM | 6819 | CB | TRP | E | 52 | −9.599 | 36.173 | −10.031 | 1.00 | 50.00 | C |
| ATOM | 6820 | CG | TRP | E | 52 | −10.357 | 36.290 | −11.335 | 1.00 | 50.00 | C |
| ATOM | 6821 | CD1 | TRP | E | 52 | −11.635 | 36.800 | −11.533 | 1.00 | 50.00 | C |
| ATOM | 6822 | CD2 | TRP | E | 52 | −10.170 | 35.421 | −12.400 | 1.00 | 50.00 | C |
| ATOM | 6823 | NE1 | TRP | E | 52 | −12.258 | 36.256 | −12.635 | 1.00 | 50.00 | N |
| ATOM | 6824 | CE2 | TRP | E | 52 | −11.443 | 35.183 | −12.914 | 1.00 | 50.00 | C |
| ATOM | 6825 | CE3 | TRP | E | 52 | −9.221 | 34.479 | −12.569 | 1.00 | 50.00 | C |
| ATOM | 6826 | CZ2 | TRP | E | 52 | −11.780 | 33.928 | −13.284 | 1.00 | 50.00 | C |
| ATOM | 6827 | CZ3 | TRP | E | 52 | −9.547 | 33.264 | −13.052 | 1.00 | 50.00 | C |
| ATOM | 6828 | CH2 | TRP | E | 52 | −10.842 | 32.961 | −13.271 | 1.00 | 50.00 | C |
| ATOM | 6829 | N | TYR | E | 53 | −8.855 | 33.038 | −9.776 | 1.00 | 50.00 | N |
| ATOM | 6830 | CA | TYR | E | 53 | −8.805 | 31.628 | −10.164 | 1.00 | 50.00 | C |
| ATOM | 6831 | C | TYR | E | 53 | −9.289 | 30.776 | −8.999 | 1.00 | 50.00 | C |
| ATOM | 6832 | O | TYR | E | 53 | −9.288 | 31.121 | −7.817 | 1.00 | 50.00 | O |
| ATOM | 6833 | CB | TYR | E | 53 | −7.445 | 31.157 | −10.684 | 1.00 | 50.00 | C |
| ATOM | 6834 | CG | TYR | E | 53 | −7.286 | 29.721 | −11.137 | 1.00 | 50.00 | C |
| ATOM | 6835 | CD1 | TYR | E | 53 | −7.684 | 29.412 | −12.386 | 1.00 | 50.00 | C |
| ATOM | 6836 | CD2 | TYR | E | 53 | −6.850 | 28.720 | −10.345 | 1.00 | 50.00 | C |
| ATOM | 6837 | CE1 | TYR | E | 53 | −7.391 | 28.212 | −12.925 | 1.00 | 50.00 | C |
| ATOM | 6838 | CE2 | TYR | E | 53 | −6.609 | 27.496 | −10.856 | 1.00 | 50.00 | C |
| ATOM | 6839 | CZ | TYR | E | 53 | −6.818 | 27.261 | −12.165 | 1.00 | 50.00 | C |
| ATOM | 6840 | OH | TYR | E | 53 | −6.596 | 26.011 | −12.679 | 1.00 | 50.00 | O |
| ATOM | 6841 | N | THR | E | 54 | −9.774 | 29.635 | −9.452 | 1.00 | 50.00 | N |
| ATOM | 6842 | CA | THR | E | 54 | −10.464 | 28.645 | −8.632 | 1.00 | 50.00 | C |
| ATOM | 6843 | C | THR | E | 54 | −10.242 | 27.299 | −9.311 | 1.00 | 50.00 | C |
| ATOM | 6844 | O | THR | E | 54 | −10.165 | 27.144 | −10.530 | 1.00 | 50.00 | O |
| ATOM | 6845 | CB | THR | E | 54 | −11.919 | 29.072 | −8.436 | 1.00 | 50.00 | C |
| ATOM | 6846 | OG1 | THR | E | 54 | −12.640 | 28.023 | −7.806 | 1.00 | 50.00 | O |
| ATOM | 6847 | CG2 | THR | E | 54 | −12.685 | 29.547 | −9.671 | 1.00 | 50.00 | C |
| ATOM | 6848 | N | SER | E | 55 | −10.155 | 26.307 | −8.443 | 1.00 | 50.00 | N |
| ATOM | 6849 | CA | SER | E | 55 | −10.091 | 24.895 | −8.815 | 1.00 | 50.00 | C |
| ATOM | 6850 | C | SER | E | 55 | −10.659 | 24.023 | −7.700 | 1.00 | 50.00 | C |
| ATOM | 6851 | O | SER | E | 55 | −10.968 | 24.478 | −6.599 | 1.00 | 50.00 | O |
| ATOM | 6852 | CB | SER | E | 55 | −8.657 | 24.529 | −9.195 | 1.00 | 50.00 | C |
| ATOM | 6853 | OG | SER | E | 55 | −8.315 | 25.009 | −10.486 | 1.00 | 50.00 | O |
| ATOM | 6854 | N | VAL | E | 56 | −10.814 | 22.744 | −8.003 | 1.00 | 50.00 | N |
| ATOM | 6855 | CA | VAL | E | 56 | −11.324 | 21.730 | −7.078 | 1.00 | 50.00 | C |
| ATOM | 6856 | C | VAL | E | 56 | −10.457 | 20.481 | −7.222 | 1.00 | 50.00 | C |
| ATOM | 6857 | O | VAL | E | 56 | −10.153 | 20.006 | −8.315 | 1.00 | 50.00 | O |
| ATOM | 6858 | CB | VAL | E | 56 | −12.829 | 21.513 | −7.267 | 1.00 | 50.00 | C |
| ATOM | 6859 | CG1 | VAL | E | 56 | −13.417 | 20.342 | −6.479 | 1.00 | 50.00 | C |
| ATOM | 6860 | CG2 | VAL | E | 56 | −13.648 | 22.742 | −6.872 | 1.00 | 50.00 | C |
| ATOM | 6861 | N | ILE | E | 57 | −10.112 | 19.920 | −6.075 | 1.00 | 50.00 | N |
| ATOM | 6862 | CA | ILE | E | 57 | −9.505 | 18.601 | −5.884 | 1.00 | 50.00 | C |
| ATOM | 6863 | C | ILE | E | 57 | −10.595 | 17.819 | −5.160 | 1.00 | 50.00 | C |
| ATOM | 6864 | O | ILE | E | 57 | −11.012 | 18.169 | −4.056 | 1.00 | 50.00 | O |
| ATOM | 6865 | CB | ILE | E | 57 | −8.174 | 18.609 | −5.124 | 1.00 | 50.00 | C |
| ATOM | 6866 | CG1 | ILE | E | 57 | −7.016 | 19.250 | −5.892 | 1.00 | 50.00 | C |
| ATOM | 6867 | CG2 | ILE | E | 57 | −7.739 | 17.267 | −4.535 | 1.00 | 50.00 | C |
| ATOM | 6868 | CD1 | ILE | E | 57 | −5.692 | 19.335 | −5.132 | 1.00 | 50.00 | C |
| ATOM | 6869 | N | THR | E | 58 | −10.972 | 16.697 | −5.750 | 1.00 | 50.00 | N |
| ATOM | 6870 | CA | THR | E | 58 | −11.828 | 15.690 | −5.124 | 1.00 | 50.00 | C |
| ATOM | 6871 | C | THR | E | 58 | −10.941 | 14.508 | −4.750 | 1.00 | 50.00 | C |
| ATOM | 6872 | O | THR | E | 58 | −10.388 | 13.825 | −5.612 | 1.00 | 50.00 | O |
| ATOM | 6873 | CB | THR | E | 58 | −12.980 | 15.352 | −6.071 | 1.00 | 50.00 | C |
| ATOM | 6874 | OG1 | THR | E | 58 | −13.823 | 16.487 | −6.212 | 1.00 | 50.00 | O |
| ATOM | 6875 | CG2 | THR | E | 58 | −13.869 | 14.195 | −5.617 | 1.00 | 50.00 | C |
| ATOM | 6876 | N | ILE | E | 59 | −10.851 | 14.245 | −3.457 | 1.00 | 50.00 | N |
| ATOM | 6877 | CA | ILE | E | 59 | −10.133 | 13.106 | −2.884 | 1.00 | 50.00 | C |
| ATOM | 6878 | C | ILE | E | 59 | −11.109 | 11.945 | −2.735 | 1.00 | 50.00 | C |
| ATOM | 6879 | O | ILE | E | 59 | −12.270 | 12.079 | −2.352 | 1.00 | 50.00 | O |
| ATOM | 6880 | CB | ILE | E | 59 | −9.458 | 13.476 | −1.561 | 1.00 | 50.00 | C |
| ATOM | 6881 | CG1 | ILE | E | 59 | −8.334 | 14.484 | −1.799 | 1.00 | 50.00 | C |
| ATOM | 6882 | CG2 | ILE | E | 59 | −8.873 | 12.334 | −0.729 | 1.00 | 50.00 | C |
| ATOM | 6883 | CD1 | ILE | E | 59 | −8.095 | 15.375 | −0.588 | 1.00 | 50.00 | C |
| ATOM | 6884 | N | GLU | E | 60 | −10.541 | 10.779 | −2.991 | 1.00 | 50.00 | N |
| ATOM | 6885 | CA | GLU | E | 60 | −11.211 | 9.491 | −2.833 | 1.00 | 50.00 | C |
| ATOM | 6886 | C | GLU | E | 60 | −10.478 | 8.752 | −1.720 | 1.00 | 50.00 | C |
| ATOM | 6887 | O | GLU | E | 60 | −9.348 | 8.288 | −1.865 | 1.00 | 50.00 | O |
| ATOM | 6888 | CB | GLU | E | 60 | −11.165 | 8.779 | −4.184 | 1.00 | 50.00 | C |
| ATOM | 6889 | CG | GLU | E | 60 | −11.937 | 9.402 | −5.346 | 1.00 | 50.00 | C |
| ATOM | 6890 | CD | GLU | E | 60 | −13.448 | 9.322 | −5.197 | 1.00 | 50.00 | C |
| ATOM | 6891 | OE1 | GLU | E | 60 | −14.063 | 10.339 | −4.811 | 1.00 | 50.00 | O |
| ATOM | 6892 | OE2 | GLU | E | 60 | −14.027 | 8.246 | −5.461 | 1.00 | 50.00 | O |
| ATOM | 6893 | N | LEU | E | 61 | −11.135 | 8.681 | −0.575 | 1.00 | 50.00 | N |
| ATOM | 6894 | CA | LEU | E | 61 | −10.593 | 8.106 | 0.657 | 1.00 | 50.00 | C |
| ATOM | 6895 | C | LEU | E | 61 | −10.426 | 6.595 | 0.744 | 1.00 | 50.00 | C |
| ATOM | 6896 | O | LEU | E | 61 | −9.462 | 6.138 | 1.358 | 1.00 | 50.00 | O |
| ATOM | 6897 | CB | LEU | E | 61 | −11.483 | 8.562 | 1.814 | 1.00 | 50.00 | C |
| ATOM | 6898 | CG | LEU | E | 61 | −11.672 | 10.063 | 2.036 | 1.00 | 50.00 | C |
| ATOM | 6899 | CD1 | LEU | E | 61 | −12.827 | 10.277 | 3.013 | 1.00 | 50.00 | C |
| ATOM | 6900 | CD2 | LEU | E | 61 | −10.283 | 10.591 | 2.386 | 1.00 | 50.00 | C |
| ATOM | 6901 | N | SER | E | 62 | −11.351 | 5.841 | 0.174 | 1.00 | 50.00 | N |
| ATOM | 6902 | CA | SER | E | 62 | −11.290 | 4.380 | 0.136 | 1.00 | 50.00 | C |
| ATOM | 6903 | C | SER | E | 62 | −10.533 | 3.976 | −1.124 | 1.00 | 50.00 | C |
| ATOM | 6904 | O | SER | E | 62 | −9.306 | 4.061 | −1.100 | 1.00 | 50.00 | O |
| ATOM | 6905 | CB | SER | E | 62 | −12.704 | 3.852 | 0.377 | 1.00 | 50.00 | C |
| ATOM | 6906 | OG | SER | E | 62 | −13.155 | 4.155 | 1.690 | 1.00 | 50.00 | O |
| ATOM | 6907 | N | ASN | E | 63 | −11.202 | 3.578 | −2.196 | 1.00 | 50.00 | N |
| ATOM | 6908 | CA | ASN | E | 63 | −10.612 | 3.130 | −3.460 | 1.00 | 50.00 | C |
| ATOM | 6909 | C | ASN | E | 63 | −10.232 | 1.659 | −3.567 | 1.00 | 50.00 | C |
| ATOM | 6910 | O | ASN | E | 63 | −10.398 | 1.072 | −4.636 | 1.00 | 50.00 | O |
| ATOM | 6911 | CB | ASN | E | 63 | −9.480 | 3.936 | −4.098 | 1.00 | 50.00 | C |
| ATOM | 6912 | CG | ASN | E | 63 | −9.864 | 5.334 | −4.556 | 1.00 | 50.00 | C |
| ATOM | 6913 | OD1 | ASN | E | 63 | −10.768 | 5.534 | −5.366 | 1.00 | 50.00 | O |
| ATOM | 6914 | ND2 | ASN | E | 63 | −9.159 | 6.325 | −4.044 | 1.00 | 50.00 | N |
| ATOM | 6915 | N | ILE | E | 64 | −9.653 | 1.082 | −2.528 | 1.00 | 50.00 | N |
| ATOM | 6916 | CA | ILE | E | 64 | −9.023 | −0.237 | −2.578 | 1.00 | 50.00 | C |
| ATOM | 6917 | C | ILE | E | 64 | −10.140 | −1.264 | −2.420 | 1.00 | 50.00 | C |
| ATOM | 6918 | O | ILE | E | 64 | −11.007 | −1.175 | −1.551 | 1.00 | 50.00 | O |
| ATOM | 6919 | CB | ILE | E | 64 | −7.901 | −0.305 | −1.540 | 1.00 | 50.00 | C |
| ATOM | 6920 | CG1 | ILE | E | 64 | −6.805 | 0.750 | −1.704 | 1.00 | 50.00 | C |
| ATOM | 6921 | CG2 | ILE | E | 64 | −7.340 | −1.718 | −1.382 | 1.00 | 50.00 | C |
| ATOM | 6922 | CD1 | ILE | E | 64 | −5.991 | 0.996 | −0.439 | 1.00 | 50.00 | C |
| ATOM | 6923 | N | LYS | E | 65 | −10.102 | −2.238 | −3.313 | 1.00 | 50.00 | N |
| ATOM | 6924 | CA | LYS | E | 65 | −11.245 | −3.083 | −3.658 | 1.00 | 50.00 | C |
| ATOM | 6925 | C | LYS | E | 65 | −11.822 | −4.045 | −2.629 | 1.00 | 50.00 | C |
| ATOM | 6926 | O | LYS | E | 65 | −12.983 | −4.432 | −2.759 | 1.00 | 50.00 | O |
| ATOM | 6927 | CB | LYS | E | 65 | −10.863 | −3.886 | −4.902 | 1.00 | 50.00 | C |
| ATOM | 6928 | CG | LYS | E | 65 | −10.805 | −3.020 | −6.158 | 1.00 | 50.00 | C |
| ATOM | 6929 | CD | LYS | E | 65 | −10.490 | −3.824 | −7.417 | 1.00 | 50.00 | C |
| ATOM | 6930 | CE | LYS | E | 65 | −10.431 | −2.889 | −8.622 | 1.00 | 50.00 | C |
| ATOM | 6931 | NZ | LYS | E | 65 | −10.200 | −3.653 | −9.857 | 1.00 | 50.00 | N |
| ATOM | 6932 | N | LYS | E | 66 | −11.029 | −4.480 | −1.665 | 1.00 | 50.00 | N |
| ATOM | 6933 | CA | LYS | E | 66 | −11.398 | −5.530 | −0.716 | 1.00 | 50.00 | C |
| ATOM | 6934 | C | LYS | E | 66 | −11.548 | −6.959 | −1.229 | 1.00 | 50.00 | C |
| ATOM | 6935 | O | LYS | E | 66 | −12.259 | −7.768 | −0.633 | 1.00 | 50.00 | O |
| ATOM | 6936 | CB | LYS | E | 66 | −12.533 | −5.168 | 0.241 | 1.00 | 50.00 | C |
| ATOM | 6937 | CG | LYS | E | 66 | −12.420 | −3.888 | 1.066 | 1.00 | 50.00 | C |
| ATOM | 6938 | CD | LYS | E | 66 | −13.768 | −3.650 | 1.745 | 1.00 | 50.00 | C |
| ATOM | 6939 | CE | LYS | E | 66 | −13.792 | −2.388 | 2.602 | 1.00 | 50.00 | C |
| ATOM | 6940 | NZ | LYS | E | 66 | −15.017 | −2.193 | 3.388 | 1.00 | 50.00 | N |
| ATOM | 6941 | N | ASN | E | 67 | −10.836 | −7.337 | −2.277 | 1.00 | 50.00 | N |
| ATOM | 6942 | CA | ASN | E | 67 | −10.909 | −8.664 | −2.887 | 1.00 | 50.00 | C |
| ATOM | 6943 | C | ASN | E | 67 | −10.352 | −9.752 | −1.974 | 1.00 | 50.00 | C |
| ATOM | 6944 | O | ASN | E | 67 | −9.151 | −9.757 | −1.708 | 1.00 | 50.00 | O |
| ATOM | 6945 | CB | ASN | E | 67 | −10.261 | −8.565 | −4.269 | 1.00 | 50.00 | C |
| ATOM | 6946 | CG | ASN | E | 67 | −8.751 | −8.384 | −4.357 | 1.00 | 50.00 | C |
| ATOM | 6947 | OD1 | ASN | E | 67 | −8.047 | −9.368 | −4.578 | 1.00 | 50.00 | O |
| ATOM | 6948 | ND2 | ASN | E | 67 | −8.220 | −7.192 | −4.152 | 1.00 | 50.00 | N |
| ATOM | 6949 | N | LYS | E | 68 | −11.205 | −10.636 | −1.480 | 1.00 | 50.00 | N |
| ATOM | 6950 | CA | LYS | E | 68 | −10.887 | −11.679 | −0.502 | 1.00 | 50.00 | C |
| ATOM | 6951 | C | LYS | E | 68 | −11.123 | −11.184 | 0.923 | 1.00 | 50.00 | C |
| ATOM | 6952 | O | LYS | E | 68 | −11.670 | −10.104 | 1.147 | 1.00 | 50.00 | O |
| ATOM | 6953 | CB | LYS | E | 68 | −9.602 | −12.465 | −0.783 | 1.00 | 50.00 | C |
| ATOM | 6954 | CG | LYS | E | 68 | −9.521 | −13.080 | −2.180 | 1.00 | 50.00 | C |
| ATOM | 6955 | CD | LYS | E | 68 | −8.136 | −13.541 | −2.629 | 1.00 | 50.00 | C |
| ATOM | 6956 | CE | LYS | E | 68 | −8.197 | −14.063 | −4.064 | 1.00 | 50.00 | C |
| ATOM | 6957 | NZ | LYS | E | 68 | −6.910 | −14.614 | −4.511 | 1.00 | 50.00 | N |
| ATOM | 6958 | N | CYS | E | 69 | −10.802 | −11.930 | 1.968 | 1.00 | 50.00 | N |
| ATOM | 6959 | CA | CYS | E | 69 | −9.764 | −11.569 | 2.934 | 1.00 | 50.00 | C |
| ATOM | 6960 | C | CYS | E | 69 | −9.756 | −10.138 | 3.458 | 1.00 | 50.00 | C |
| ATOM | 6961 | O | CYS | E | 69 | −10.084 | −9.911 | 4.622 | 1.00 | 50.00 | O |
| ATOM | 6962 | CB | CYS | E | 69 | −8.410 | −12.107 | 2.470 | 1.00 | 50.00 | C |
| ATOM | 6963 | SG | CYS | E | 69 | −8.599 | −13.888 | 2.292 | 1.00 | 50.00 | S |
| ATOM | 6964 | N | ASN | E | 70 | −9.330 | −9.214 | 2.611 | 1.00 | 50.00 | N |
| ATOM | 6965 | CA | ASN | E | 70 | −9.243 | −7.772 | 2.844 | 1.00 | 50.00 | C |
| ATOM | 6966 | C | ASN | E | 70 | −10.224 | −7.062 | 3.771 | 1.00 | 50.00 | C |
| ATOM | 6967 | O | ASN | E | 70 | −9.796 | −6.142 | 4.467 | 1.00 | 50.00 | O |
| ATOM | 6968 | CB | ASN | E | 70 | −9.451 | −7.115 | 1.481 | 1.00 | 50.00 | C |
| ATOM | 6969 | CG | ASN | E | 70 | −8.312 | −6.954 | 0.488 | 1.00 | 50.00 | C |
| ATOM | 6970 | OD1 | ASN | E | 70 | −7.926 | −7.863 | −0.246 | 1.00 | 50.00 | O |
| ATOM | 6971 | ND2 | ASN | E | 70 | −7.833 | −5.729 | 0.384 | 1.00 | 50.00 | N |
| ATOM | 6972 | N | GLY | E | 71 | −11.500 | −7.398 | 3.663 | 1.00 | 50.00 | N |
| ATOM | 6973 | CA | GLY | E | 71 | −12.604 | −6.787 | 4.404 | 1.00 | 50.00 | C |
| ATOM | 6974 | C | GLY | E | 71 | −12.329 | −6.325 | 5.826 | 1.00 | 50.00 | C |
| ATOM | 6975 | O | GLY | E | 71 | −12.456 | −5.146 | 6.149 | 1.00 | 50.00 | O |
| ATOM | 6976 | N | THR | E | 72 | −11.807 | −7.270 | 6.586 | 1.00 | 50.00 | N |
| ATOM | 6977 | CA | THR | E | 72 | −11.103 | −7.098 | 7.854 | 1.00 | 50.00 | C |
| ATOM | 6978 | C | THR | E | 72 | −10.425 | −5.768 | 8.176 | 1.00 | 50.00 | C |
| ATOM | 6979 | O | THR | E | 72 | −10.866 | −5.071 | 9.089 | 1.00 | 50.00 | O |
| ATOM | 6980 | CB | THR | E | 72 | −10.260 | −8.375 | 7.794 | 1.00 | 50.00 | C |
| ATOM | 6981 | OG1 | THR | E | 72 | −11.008 | −9.582 | 7.761 | 1.00 | 50.00 | O |
| ATOM | 6982 | CG2 | THR | E | 72 | −9.490 | −8.530 | 9.095 | 1.00 | 50.00 | C |
| ATOM | 6983 | N | ASP | E | 73 | −9.359 | −5.396 | 7.489 | 1.00 | 50.00 | N |
| ATOM | 6984 | CA | ASP | E | 73 | −8.532 | −4.240 | 7.845 | 1.00 | 50.00 | C |
| ATOM | 6985 | C | ASP | E | 73 | −8.969 | −2.926 | 7.221 | 1.00 | 50.00 | C |
| ATOM | 6986 | O | ASP | E | 73 | −9.090 | −1.936 | 7.940 | 1.00 | 50.00 | O |
| ATOM | 6987 | CB | ASP | E | 73 | −7.079 | −4.495 | 7.457 | 1.00 | 50.00 | C |
| ATOM | 6988 | CG | ASP | E | 73 | −6.574 | −5.583 | 8.389 | 1.00 | 50.00 | C |
| ATOM | 6989 | OD1 | ASP | E | 73 | −6.142 | −5.265 | 9.518 | 1.00 | 50.00 | O |
| ATOM | 6990 | OD2 | ASP | E | 73 | −6.590 | −6.760 | 7.968 | 1.00 | 50.00 | O |
| ATOM | 6991 | N | ALA | E | 74 | −9.337 | −2.919 | 5.953 | 1.00 | 50.00 | N |
| ATOM | 6992 | CA | ALA | E | 74 | −10.334 | −1.986 | 5.434 | 1.00 | 50.00 | C |
| ATOM | 6993 | C | ALA | E | 74 | −11.500 | −1.594 | 6.338 | 1.00 | 50.00 | C |
| ATOM | 6994 | O | ALA | E | 74 | −11.705 | −0.393 | 6.505 | 1.00 | 50.00 | O |
| ATOM | 6995 | CB | ALA | E | 74 | −10.752 | −2.673 | 4.138 | 1.00 | 50.00 | C |
| ATOM | 6996 | N | LYS | E | 75 | −12.166 | −2.484 | 7.059 | 1.00 | 50.00 | N |
| ATOM | 6997 | CA | LYS | E | 75 | −13.089 | −2.132 | 8.141 | 1.00 | 50.00 | C |
| ATOM | 6998 | C | LYS | E | 75 | −12.482 | −1.208 | 9.188 | 1.00 | 50.00 | C |
| ATOM | 6999 | O | LYS | E | 75 | −12.900 | −0.052 | 9.194 | 1.00 | 50.00 | O |
| ATOM | 7000 | CB | LYS | E | 75 | −13.745 | −3.304 | 8.869 | 1.00 | 50.00 | C |
| ATOM | 7001 | CG | LYS | E | 75 | −14.778 | −4.053 | 8.033 | 1.00 | 50.00 | C |
| ATOM | 7002 | CD | LYS | E | 75 | −15.238 | −5.325 | 8.741 | 1.00 | 50.00 | C |
| ATOM | 7003 | CE | LYS | E | 75 | −16.010 | −6.222 | 7.776 | 1.00 | 50.00 | C |
| ATOM | 7004 | NZ | LYS | E | 75 | −16.612 | −7.343 | 8.511 | 1.00 | 50.00 | N |
| ATOM | 7005 | N | VAL | E | 76 | −11.472 | −1.622 | 9.936 | 1.00 | 50.00 | N |
| ATOM | 7006 | CA | VAL | E | 76 | −10.603 | −0.753 | 10.732 | 1.00 | 50.00 | C |
| ATOM | 7007 | C | VAL | E | 76 | −10.348 | 0.648 | 10.185 | 1.00 | 50.00 | C |
| ATOM | 7008 | O | VAL | E | 76 | −10.463 | −1.645 | 10.893 | 1.00 | 50.00 | O |
| ATOM | 7009 | CB | VAL | E | 76 | −9.345 | −1.553 | 11.076 | 1.00 | 50.00 | C |
| ATOM | 7010 | CG1 | VAL | E | 76 | −8.332 | −0.678 | 11.806 | 1.00 | 50.00 | C |
| ATOM | 7011 | CG2 | VAL | E | 76 | −9.577 | −2.840 | 11.869 | 1.00 | 50.00 | C |
| ATOM | 7012 | N | LYS | E | 77 | −10.024 | 0.742 | 8.909 | 1.00 | 50.00 | N |
| ATOM | 7013 | CA | LYS | E | 77 | −9.617 | 1.965 | 8.218 | 1.00 | 50.00 | C |
| ATOM | 7014 | C | LYS | E | 77 | −10.770 | 2.918 | 7.955 | 1.00 | 50.00 | C |
| ATOM | 7015 | O | LYS | E | 77 | −10.805 | 3.995 | 8.546 | 1.00 | 50.00 | O |
| ATOM | 7016 | CB | LYS | E | 77 | −8.959 | 1.532 | 6.909 | 1.00 | 50.00 | C |
| ATOM | 7017 | CG | LYS | E | 77 | −7.731 | 0.729 | 7.317 | 1.00 | 50.00 | C |
| ATOM | 7018 | CD | LYS | E | 77 | −6.886 | 0.267 | 6.143 | 1.00 | 50.00 | C |
| ATOM | 7019 | CE | LYS | E | 77 | −5.788 | −0.569 | 6.788 | 1.00 | 50.00 | C |
| ATOM | 7020 | NZ | LYS | E | 77 | −4.571 | −0.691 | 5.984 | 1.00 | 50.00 | N |
| ATOM | 7021 | N | LEU | E | 78 | −11.719 | 2.458 | 7.159 | 1.00 | 50.00 | N |
| ATOM | 7022 | CA | LEU | E | 78 | −13.038 | 3.056 | 6.966 | 1.00 | 50.00 | C |
| ATOM | 7023 | C | LEU | E | 78 | −13.865 | 3.330 | 8.219 | 1.00 | 50.00 | C |
| ATOM | 7024 | O | LEU | E | 78 | −14.727 | 4.207 | 8.170 | 1.00 | 50.00 | O |
| ATOM | 7025 | CB | LEU | E | 78 | −13.750 | 2.110 | 5.998 | 1.00 | 50.00 | C |
| ATOM | 7026 | CG | LEU | E | 78 | −13.294 | 2.014 | 4.540 | 1.00 | 50.00 | C |
| ATOM | 7027 | CD1 | LEU | E | 78 | −11.913 | 1.456 | 4.194 | 1.00 | 50.00 | C |
| ATOM | 7028 | CD2 | LEU | E | 78 | −14.312 | 1.144 | 3.805 | 1.00 | 50.00 | C |
| ATOM | 7029 | N | ILE | E | 79 | −13.573 | 2.695 | 9.345 | 1.00 | 50.00 | N |
| ATOM | 7030 | CA | ILE | E | 79 | −13.905 | 3.153 | 10.695 | 1.00 | 50.00 | C |
| ATOM | 7031 | C | ILE | E | 79 | −13.148 | 4.463 | 10.884 | 1.00 | 50.00 | C |
| ATOM | 7032 | O | ILE | E | 79 | −13.763 | 5.513 | 10.708 | 1.00 | 50.00 | O |
| ATOM | 7033 | CB | ILE | E | 79 | −13.710 | 2.068 | 11.760 | 1.00 | 50.00 | C |
| ATOM | 7034 | CG1 | ILE | E | 79 | −14.585 | 0.826 | 11.576 | 1.00 | 50.00 | C |
| ATOM | 7035 | CG2 | ILE | E | 79 | −13.894 | 2.546 | 13.201 | 1.00 | 50.00 | C |
| ATOM | 7036 | CD1 | ILE | E | 79 | −14.132 | −0.434 | 12.311 | 1.00 | 50.00 | C |
| ATOM | 7037 | N | LYS | E | 80 | −11.859 | 4.455 | 11.176 | 1.00 | 50.00 | N |
| ATOM | 7038 | CA | LYS | E | 80 | −11.043 | 5.616 | 11.538 | 1.00 | 50.00 | C |
| ATOM | 7039 | C | LYS | E | 80 | −10.998 | 6.853 | 10.650 | 1.00 | 50.00 | C |
| ATOM | 7040 | O | LYS | E | 80 | −10.923 | 7.969 | 11.159 | 1.00 | 50.00 | O |
| ATOM | 7041 | CB | LYS | E | 80 | −9.618 | 5.104 | 11.729 | 1.00 | 50.00 | C |
| ATOM | 7042 | CG | LYS | E | 80 | −9.568 | 4.009 | 12.795 | 1.00 | 50.00 | C |
| ATOM | 7043 | CD | LYS | E | 80 | −8.121 | 3.587 | 13.005 | 1.00 | 50.00 | C |
| ATOM | 7044 | CE | LYS | E | 80 | −7.759 | 2.405 | 13.899 | 1.00 | 50.00 | C |
| ATOM | 7045 | NZ | LYS | E | 80 | −6.331 | 2.108 | 14.074 | 1.00 | 50.00 | N |
| ATOM | 7046 | N | GLN | E | 81 | −11.063 | 6.661 | 9.344 | 1.00 | 50.00 | N |
| ATOM | 7047 | CA | GLN | E | 81 | −11.507 | 7.635 | 8.346 | 1.00 | 50.00 | C |
| ATOM | 7048 | C | GLN | E | 81 | −12.739 | 8.458 | 8.707 | 1.00 | 50.00 | C |
| ATOM | 7049 | O | GLN | E | 81 | −12.695 | 9.685 | 8.634 | 1.00 | 50.00 | O |
| ATOM | 7050 | CB | GLN | E | 81 | −11.743 | 6.854 | 7.052 | 1.00 | 50.00 | C |
| ATOM | 7051 | CG | GLN | E | 81 | −10.525 | 6.227 | 6.375 | 1.00 | 50.00 | C |
| ATOM | 7052 | CD | GLN | E | 81 | −9.531 | 7.185 | 5.736 | 1.00 | 50.00 | C |
| ATOM | 7053 | OE1 | GLN | E | 81 | −9.664 | 8.408 | 5.748 | 1.00 | 50.00 | O |
| ATOM | 7054 | NE2 | GLN | E | 81 | −8.457 | 6.625 | 5.210 | 1.00 | 50.00 | N |
| ATOM | 7055 | N | GLU | E | 82 | −13.805 | 7.799 | 9.129 | 1.00 | 50.00 | N |
| ATOM | 7056 | CA | GLU | E | 82 | −14.969 | 8.407 | 9.771 | 1.00 | 50.00 | C |
| ATOM | 7057 | C | GLU | E | 82 | −14.820 | 8.970 | 11.177 | 1.00 | 50.00 | C |
| ATOM | 7058 | O | GLU | E | 82 | −15.404 | 10.014 | 11.461 | 1.00 | 50.00 | O |
| ATOM | 7059 | CB | GLU | E | 82 | −16.143 | 7.429 | 9.700 | 1.00 | 50.00 | C |
| ATOM | 7060 | CG | GLU | E | 82 | −16.730 | 7.298 | 8.295 | 1.00 | 50.00 | C |
| ATOM | 7061 | CD | GLU | E | 82 | −17.231 | 8.673 | 7.876 | 1.00 | 50.00 | C |
| ATOM | 7062 | OE1 | GLU | E | 82 | −16.637 | 9.294 | 6.969 | 1.00 | 50.00 | O |
| ATOM | 7063 | OE2 | GLU | E | 82 | −18.093 | 9.233 | 8.587 | 1.00 | 50.00 | O |
| ATOM | 7064 | N | LEU | E | 83 | −14.044 | 8.355 | 12.052 | 1.00 | 50.00 | N |
| ATOM | 7065 | CA | LEU | E | 83 | −13.716 | 8.917 | 13.362 | 1.00 | 50.00 | C |
| ATOM | 7066 | C | LEU | E | 83 | −12.892 | 10.197 | 13.262 | 1.00 | 50.00 | C |
| ATOM | 7067 | O | LEU | E | 83 | −13.225 | 11.139 | 13.981 | 1.00 | 50.00 | O |
| ATOM | 7068 | CB | LEU | E | 83 | −13.092 | 7.828 | 14.236 | 1.00 | 50.00 | C |
| ATOM | 7069 | CG | LEU | E | 83 | −13.965 | 6.670 | 14.726 | 1.00 | 50.00 | C |
| ATOM | 7070 | CD1 | LEU | E | 83 | −14.660 | 5.855 | 13.638 | 1.00 | 50.00 | C |
| ATOM | 7071 | CD2 | LEU | E | 83 | −13.121 | 5.642 | 15.476 | 1.00 | 50.00 | C |
| ATOM | 7072 | N | ASP | E | 84 | −11.956 | 10.296 | 12.330 | 1.00 | 50.00 | N |
| ATOM | 7073 | CA | ASP | E | 84 | −11.397 | 11.540 | 11.800 | 1.00 | 50.00 | C |
| ATOM | 7074 | C | ASP | E | 84 | −12.484 | 12.463 | 11.257 | 1.00 | 50.00 | C |
| ATOM | 7075 | O | ASP | E | 84 | −12.983 | 13.333 | 11.970 | 1.00 | 50.00 | O |
| ATOM | 7076 | CB | ASP | E | 84 | −10.315 | 11.283 | 10.747 | 1.00 | 50.00 | C |
| ATOM | 7077 | CG | ASP | E | 84 | −8.955 | 10.725 | 11.132 | 1.00 | 50.00 | C |
| ATOM | 7078 | OD1 | ASP | E | 84 | −8.263 | 11.363 | 11.953 | 1.00 | 50.00 | O |
| ATOM | 7079 | OD2 | ASP | E | 84 | −8.556 | 9.671 | 10.590 | 1.00 | 50.00 | O |
| ATOM | 7080 | N | LYS | E | 85 | −12.912 | 12.269 | 10.021 | 1.00 | 50.00 | N |
| ATOM | 7081 | CA | LYS | E | 85 | −13.611 | 13.279 | 9.227 | 1.00 | 50.00 | C |
| ATOM | 7082 | C | LYS | E | 85 | −15.029 | 13.684 | 9.589 | 1.00 | 50.00 | C |
| ATOM | 7083 | O | LYS | E | 85 | −15.442 | 14.743 | 9.118 | 1.00 | 50.00 | O |
| ATOM | 7084 | CB | LYS | E | 85 | −13.731 | 12.769 | 7.793 | 1.00 | 50.00 | C |
| ATOM | 7085 | CG | LYS | E | 85 | −12.359 | 12.813 | 7.130 | 1.00 | 50.00 | C |
| ATOM | 7086 | CD | LYS | E | 85 | −12.540 | 12.507 | 5.648 | 1.00 | 50.00 | C |
| ATOM | 7087 | CE | LYS | E | 85 | −11.171 | 12.719 | 5.011 | 1.00 | 50.00 | C |
| ATOM | 7088 | NZ | LYS | E | 85 | −11.483 | 13.045 | 3.615 | 1.00 | 50.00 | N |
| ATOM | 7089 | N | TYR | E | 86 | −15.756 | 12.883 | 10.350 | 1.00 | 50.00 | N |
| ATOM | 7090 | CA | TYR | E | 86 | −17.105 | 13.210 | 10.809 | 1.00 | 50.00 | C |
| ATOM | 7091 | C | TYR | E | 86 | −17.343 | 14.657 | 11.230 | 1.00 | 50.00 | C |
| ATOM | 7092 | O | TYR | E | 86 | −18.351 | 15.251 | 10.851 | 1.00 | 50.00 | O |
| ATOM | 7093 | CB | TYR | E | 86 | −17.466 | 12.176 | 11.876 | 1.00 | 50.00 | C |
| ATOM | 7094 | CG | TYR | E | 86 | −18.900 | 12.272 | 12.345 | 1.00 | 50.00 | C |
| ATOM | 7095 | CD1 | TYR | E | 86 | −19.923 | 11.825 | 11.586 | 1.00 | 50.00 | C |
| ATOM | 7096 | CD2 | TYR | E | 86 | −19.123 | 13.152 | 13.341 | 1.00 | 50.00 | C |
| ATOM | 7097 | CE1 | TYR | E | 86 | −21.177 | 12.264 | 11.815 | 1.00 | 50.00 | C |
| ATOM | 7098 | CE2 | TYR | E | 86 | −20.363 | 13.656 | 13.476 | 1.00 | 50.00 | C |
| ATOM | 7099 | CZ | TYR | E | 86 | −21.408 | 13.183 | 12.773 | 1.00 | 50.00 | C |
| ATOM | 7100 | OH | TYR | E | 86 | −22.624 | 13.777 | 12.990 | 1.00 | 50.00 | O |
| ATOM | 7101 | N | LYS | E | 87 | −16.432 | 15.232 | 11.992 | 1.00 | 50.00 | N |
| ATOM | 7102 | CA | LYS | E | 87 | −16.532 | 16.601 | 12.493 | 1.00 | 50.00 | C |
| ATOM | 7103 | C | LYS | E | 87 | −16.087 | 17.727 | 11.571 | 1.00 | 50.00 | C |
| ATOM | 7104 | O | LYS | E | 87 | −16.732 | 18.774 | 11.591 | 1.00 | 50.00 | O |
| ATOM | 7105 | CB | LYS | E | 87 | −15.564 | 16.664 | 13.667 | 1.00 | 50.00 | C |
| ATOM | 7106 | CG | LYS | E | 87 | −15.929 | 15.670 | 14.767 | 1.00 | 50.00 | C |
| ATOM | 7107 | CD | LYS | E | 87 | −14.930 | 16.061 | 15.845 | 1.00 | 50.00 | C |
| ATOM | 7108 | CE | LYS | E | 87 | −14.795 | 15.419 | 17.216 | 1.00 | 50.00 | C |
| ATOM | 7109 | NZ | LYS | E | 87 | −13.829 | 16.293 | 17.896 | 1.00 | 50.00 | N |
| ATOM | 7110 | N | ASN | E | 88 | −15.040 | 17.544 | 10.783 | 1.00 | 50.00 | N |
| ATOM | 7111 | CA | ASN | E | 88 | −14.860 | 18.292 | 9.538 | 1.00 | 50.00 | C |
| ATOM | 7112 | C | ASN | E | 88 | −16.100 | 18.338 | 8.660 | 1.00 | 50.00 | C |
| ATOM | 7113 | O | ASN | E | 88 | −16.549 | 19.453 | 8.400 | 1.00 | 50.00 | O |
| ATOM | 7114 | CB | ASN | E | 88 | −13.742 | 17.762 | 8.642 | 1.00 | 50.00 | C |
| ATOM | 7115 | CG | ASN | E | 88 | −12.366 | 17.977 | 9.245 | 1.00 | 50.00 | C |
| ATOM | 7116 | OD1 | ASN | E | 88 | −11.743 | 16.992 | 9.636 | 1.00 | 50.00 | O |
| ATOM | 7117 | ND2 | ASN | E | 88 | −11.986 | 19.234 | 9.382 | 1.00 | 50.00 | N |
| ATOM | 7118 | N | ALA | E | 89 | −16.701 | 17.198 | 8.357 | 1.00 | 50.00 | N |
| ATOM | 7119 | CA | ALA | E | 89 | −18.046 | 17.113 | 7.792 | 1.00 | 50.00 | C |
| ATOM | 7120 | C | ALA | E | 89 | −19.024 | 17.990 | 8.563 | 1.00 | 50.00 | C |
| ATOM | 7121 | O | ALA | E | 89 | −19.263 | 19.091 | 8.071 | 1.00 | 50.00 | O |
| ATOM | 7122 | CB | ALA | E | 89 | −18.461 | −15.653 | 7.619 | 1.00 | 50.00 | C |
| ATOM | 7123 | N | VAL | E | 90 | −19.431 | 17.692 | 9.786 | 1.00 | 50.00 | N |
| ATOM | 7124 | CA | VAL | E | 90 | −20.314 | 18.534 | 10.596 | 1.00 | 50.00 | C |
| ATOM | 7125 | C | VAL | E | 90 | −20.033 | 20.033 | 10.630 | 1.00 | 50.00 | C |
| ATOM | 7126 | O | VAL | E | 90 | −20.981 | 20.781 | 10.392 | 1.00 | 50.00 | O |
| ATOM | 7127 | CB | VAL | E | 90 | −20.460 | 17.978 | 12.014 | 1.00 | 50.00 | C |
| ATOM | 7128 | CG1 | VAL | E | 90 | −21.344 | 18.823 | 12.929 | 1.00 | 50.00 | C |
| ATOM | 7129 | CG2 | VAL | E | 90 | −21.096 | 16.591 | 12.062 | 1.00 | 50.00 | C |
| ATOM | 7130 | N | THR | E | 91 | −18.830 | 20.483 | 10.950 | 1.00 | 50.00 | N |
| ATOM | 7131 | CA | THR | E | 91 | −18.394 | 21.876 | 10.829 | 1.00 | 50.00 | C |
| ATOM | 7132 | C | THR | E | 91 | −18.841 | 22.433 | 9.483 | 1.00 | 50.00 | C |
| ATOM | 7133 | O | THR | E | 91 | −19.808 | 23.193 | 9.508 | 1.00 | 50.00 | O |
| ATOM | 7134 | CB | THR | E | 91 | −16.911 | 22.064 | 11.154 | 1.00 | 50.00 | C |
| ATOM | 7135 | OG1 | THR | E | 91 | −16.684 | 21.792 | 12.530 | 1.00 | 50.00 | O |
| ATOM | 7136 | CG2 | THR | E | 91 | −16.328 | 23.455 | 10.912 | 1.00 | 50.00 | C |
| ATOM | 7137 | N | GLU | E | 92 | −18.301 | 21.978 | 8.364 | 1.00 | 50.00 | N |
| ATOM | 7138 | CA | GLU | E | 92 | −18.803 | 22.243 | 7.015 | 1.00 | 50.00 | C |
| ATOM | 7139 | C | GLU | E | 92 | −20.294 | 22.525 | 6.869 | 1.00 | 50.00 | C |
| ATOM | 7140 | O | GLU | E | 92 | −20.661 | 23.658 | 6.563 | 1.00 | 50.00 | O |
| ATOM | 7141 | CB | GLU | E | 92 | −18.299 | 21.184 | 6.033 | 1.00 | 50.00 | C |
| ATOM | 7142 | CG | GLU | E | 92 | −16.780 | 21.072 | 5.903 | 1.00 | 50.00 | C |
| ATOM | 7143 | CD | GLU | E | 92 | −16.246 | 22.307 | 5.199 | 1.00 | 50.00 | C |
| ATOM | 7144 | OE1 | GLU | E | 92 | −15.358 | 23.006 | 5.735 | 1.00 | 50.00 | O |
| ATOM | 7145 | OE2 | GLU | E | 92 | −16.803 | 22.629 | 4.129 | 1.00 | 50.00 | O |
| ATOM | 7146 | N | LEU | E | 93 | −21.155 | 21.614 | 7.293 | 1.00 | 50.00 | N |
| ATOM | 7147 | CA | LEU | E | 93 | −22.606 | 21.796 | 7.355 | 1.00 | 50.00 | C |
| ATOM | 7148 | C | LEU | E | 93 | −23.034 | 23.067 | 8.082 | 1.00 | 50.00 | C |
| ATOM | 7149 | O | LEU | E | 93 | −23.493 | 23.972 | 7.387 | 1.00 | 50.00 | O |
| ATOM | 7150 | CB | LEU | E | 93 | −23.271 | 20.521 | 7.876 | 1.00 | 50.00 | C |
| ATOM | 7151 | CG | LEU | E | 93 | −23.237 | 19.237 | 7.043 | 1.00 | 50.00 | C |
| ATOM | 7152 | CD1 | LEU | E | 93 | −21.852 | 18.693 | 6.698 | 1.00 | 50.00 | C |
| ATOM | 7153 | CD2 | LEU | E | 93 | −23.927 | 18.111 | 7.812 | 1.00 | 50.00 | C |
| ATOM | 7154 | N | GLN | E | 94 | −22.769 | 23.235 | 9.368 | 1.00 | 50.00 | N |
| ATOM | 7155 | CA | GLN | E | 94 | −22.928 | 24.482 | 10.122 | 1.00 | 50.00 | C |
| ATOM | 7156 | C | GLN | E | 94 | −22.545 | 25.818 | 9.493 | 1.00 | 50.00 | C |
| ATOM | 7157 | O | GLN | E | 94 | −23.256 | 26.809 | 9.655 | 1.00 | 50.00 | O |
| ATOM | 7158 | CB | GLN | E | 94 | −22.220 | 24.341 | 11.472 | 1.00 | 50.00 | C |
| ATOM | 7159 | CG | GLN | E | 94 | −22.766 | 23.292 | 12.439 | 1.00 | 50.00 | C |
| ATOM | 7160 | CD | GLN | E | 94 | −24.159 | 23.586 | 12.977 | 1.00 | 50.00 | C |
| ATOM | 7161 | OE1 | GLN | E | 94 | −24.717 | 24.677 | 12.861 | 1.00 | 50.00 | O |
| ATOM | 7162 | NE2 | GLN | E | 94 | −24.772 | 22.593 | 13.591 | 1.00 | 50.00 | N |
| ATOM | 7163 | N | LEU | E | 95 | −21.438 | 25.841 | 8.769 | 1.00 | 50.00 | N |
| ATOM | 7164 | CA | LEU | E | 95 | −20.922 | 26.978 | 8.007 | 1.00 | 50.00 | C |
| ATOM | 7165 | C | LEU | E | 95 | −21.774 | 27.284 | 6.780 | 1.00 | 50.00 | C |
| ATOM | 7166 | O | LEU | E | 95 | −22.281 | 28.394 | 6.625 | 1.00 | 50.00 | O |
| ATOM | 7167 | CB | LEU | E | 95 | −19.474 | 26.678 | 7.610 | 1.00 | 50.00 | C |
| ATOM | 7168 | CG | LEU | E | 95 | −18.394 | 26.663 | 8.690 | 1.00 | 50.00 | C |
| ATOM | 7169 | CD1 | LEU | E | 95 | −18.561 | 25.803 | 9.938 | 1.00 | 50.00 | C |
| ATOM | 7170 | CD2 | LEU | E | 95 | −16.984 | 26.323 | 8.205 | 1.00 | 50.00 | C |
| ATOM | 7171 | N | LEU | E | 96 | −21.964 | 26.294 | 5.924 | 1.00 | 50.00 | N |
| ATOM | 7172 | CA | LEU | E | 96 | −22.783 | 26.343 | 4.713 | 1.00 | 50.00 | C |
| ATOM | 7173 | C | LEU | E | 96 | −24.270 | 26.631 | 4.900 | 1.00 | 50.00 | C |
| ATOM | 7174 | O | LEU | E | 96 | −24.850 | 27.278 | 4.029 | 1.00 | 50.00 | O |
| ATOM | 7175 | CB | LEU | E | 96 | −22.540 | 25.016 | 3.993 | 1.00 | 50.00 | C |
| ATOM | 7176 | CG | LEU | E | 96 | −21.235 | 24.780 | 3.230 | 1.00 | 50.00 | C |
| ATOM | 7177 | CD1 | LEU | E | 96 | −19.896 | 24.873 | 3.962 | 1.00 | 50.00 | C |
| ATOM | 7178 | CD2 | LEU | E | 96 | −21.294 | 23.400 | 2.577 | 1.00 | 50.00 | C |
| ATOM | 7179 | N | MET | E | 97 | −24.889 | 26.216 | 5.994 | 1.00 | 50.00 | N |
| ATOM | 7180 | CA | MET | E | 97 | −26.228 | 26.626 | 6.425 | 1.00 | 50.00 | C |
| ATOM | 7181 | C | MET | E | 97 | −26.540 | 28.119 | 6.452 | 1.00 | 50.00 | C |
| ATOM | 7182 | O | MET | E | 97 | −27.627 | 28.552 | 6.070 | 1.00 | 50.00 | O |
| ATOM | 7183 | CB | MET | E | 97 | −26.497 | 26.039 | 7.812 | 1.00 | 50.00 | C |
| ATOM | 7184 | CG | MET | E | 97 | −26.669 | 24.523 | 7.878 | 1.00 | 50.00 | C |
| ATOM | 7185 | SD | MET | E | 97 | −27.949 | 23.952 | 6.749 | 1.00 | 50.00 | S |
| ATOM | 7186 | CE | MET | E | 97 | −29.419 | 24.447 | 7.661 | 1.00 | 50.00 | C |
| ATOM | 7187 | N | GLN | E | 98 | −25.577 | 28.894 | 6.917 | 1.00 | 50.00 | N |
| ATOM | 7188 | CA | GLN | E | 98 | −25.630 | 30.349 | 7.040 | 1.00 | 50.00 | C |
| ATOM | 7189 | C | GLN | E | 98 | −25.107 | 31.051 | 5.793 | 1.00 | 50.00 | C |
| ATOM | 7190 | O | GLN | E | 98 | −24.680 | 30.494 | 4.781 | 1.00 | 50.00 | O |
| ATOM | 7191 | CB | GLN | E | 98 | −24.709 | 30.608 | 8.232 | 1.00 | 50.00 | C |
| ATOM | 7192 | CG | GLN | E | 98 | −25.060 | 30.265 | 9.677 | 1.00 | 50.00 | C |
| ATOM | 7193 | CD | GLN | E | 98 | −25.985 | 31.266 | 10.350 | 1.00 | 50.00 | C |
| ATOM | 7194 | OE1 | GLN | E | 98 | −26.438 | 32.262 | 9.786 | 1.00 | 50.00 | O |
| ATOM | 7195 | NE2 | GLN | E | 98 | −26.262 | 31.011 | 11.614 | 1.00 | 50.00 | N |
| ATOM | 7196 | N | LEU | F | 160 | −0.945 | −91.976 | 10.356 | 1.00 | 50.00 | N |
| ATOM | 7197 | CA | LEU | F | 160 | −0.873 | −91.623 | 8.929 | 1.00 | 50.00 | C |
| ATOM | 7198 | C | LEU | F | 160 | −2.203 | −91.512 | 8.183 | 1.00 | 50.00 | C |
| ATOM | 7199 | O | LEU | F | 160 | −2.303 | −90.606 | 7.357 | 1.00 | 50.00 | O |
| ATOM | 7200 | CB | LEU | F | 160 | 0.414 | −92.289 | 8.440 | 1.00 | 50.00 | C |
| ATOM | 7201 | CG | LEU | F | 160 | 0.374 | −93.758 | 8.020 | 1.00 | 50.00 | C |
| ATOM | 7202 | CD1 | LEU | F | 160 | −0.609 | −94.568 | 8.871 | 1.00 | 50.00 | C |
| ATOM | 7203 | CD2 | LEU | F | 160 | 0.303 | −93.606 | 6.503 | 1.00 | 50.00 | C |
| ATOM | 7204 | N | GLU | F | 161 | −3.238 | −92.298 | 8.446 | 1.00 | 50.00 | N |
| ATOM | 7205 | CA | GLU | F | 161 | −4.646 | −91.985 | 8.181 | 1.00 | 50.00 | C |
| ATOM | 7206 | C | GLU | F | 161 | −5.282 | −91.159 | 9.295 | 1.00 | 50.00 | C |
| ATOM | 7207 | O | GLU | F | 161 | −5.965 | −90.164 | 9.055 | 1.00 | 50.00 | O |
| ATOM | 7208 | CB | GLU | F | 161 | −5.569 | −93.174 | 7.900 | 1.00 | 50.00 | C |
| ATOM | 7209 | CG | GLU | F | 161 | −7.070 | −92.937 | 7.726 | 1.00 | 50.00 | C |
| ATOM | 7210 | CD | GLU | F | 161 | −7.483 | −92.033 | 6.574 | 1.00 | 50.00 | C |
| ATOM | 7211 | OE1 | GLU | F | 161 | −7.665 | −92.536 | 5.444 | 1.00 | 50.00 | O |
| ATOM | 7212 | OE2 | GLU | F | 161 | −7.647 | −90.812 | 6.788 | 1.00 | 50.00 | O |
| ATOM | 7213 | N | GLY | F | 162 | −5.153 | −91.577 | 10.543 | 1.00 | 50.00 | N |
| ATOM | 7214 | CA | GLY | F | 162 | −5.265 | −90.662 | 11.677 | 1.00 | 50.00 | C |
| ATOM | 7215 | C | GLY | F | 162 | −4.387 | −89.418 | 11.647 | 1.00 | 50.00 | C |
| ATOM | 7216 | O | GLY | F | 162 | −4.882 | −88.356 | 12.020 | 1.00 | 50.00 | O |
| ATOM | 7217 | N | GLU | F | 163 | −3.156 | −89.526 | 11.176 | 1.00 | 50.00 | N |
| ATOM | 7218 | CA | GLU | F | 163 | −2.276 | −88.375 | 10.985 | 1.00 | 50.00 | C |
| ATOM | 7219 | C | GLU | F | 163 | −2.561 | −87.584 | 9.715 | 1.00 | 50.00 | C |
| ATOM | 7220 | O | GLU | F | 163 | −2.234 | −86.407 | 9.840 | 1.00 | 50.00 | O |
| ATOM | 7221 | CB | GLU | F | 163 | −0.789 | −88.719 | 11.057 | 1.00 | 50.00 | C |
| ATOM | 7222 | CG | GLU | F | 163 | −0.209 | −89.593 | 12.166 | 1.00 | 50.00 | C |
| ATOM | 7223 | CD | GLU | F | 163 | −0.091 | −88.792 | 13.449 | 1.00 | 50.00 | C |
| ATOM | 7224 | OE1 | GLU | F | 163 | −1.062 | −88.893 | 14.228 | 1.00 | 50.00 | O |
| ATOM | 7225 | OE2 | GLU | F | 163 | 0.903 | −88.067 | 13.668 | 1.00 | 50.00 | O |
| ATOM | 7226 | N | VAL | F | 164 | −3.120 | −88.071 | 8.616 | 1.00 | 50.00 | N |
| ATOM | 7227 | CA | VAL | F | 164 | −3.669 | −87.255 | 7.529 | 1.00 | 50.00 | C |
| ATOM | 7228 | C | VAL | F | 164 | −4.731 | −86.224 | 7.906 | 1.00 | 50.00 | C |
| ATOM | 7229 | O | VAL | F | 164 | −4.858 | −85.137 | 7.343 | 1.00 | 50.00 | O |
| ATOM | 7230 | CB | VAL | F | 164 | −4.149 | −87.788 | 6.178 | 1.00 | 50.00 | C |
| ATOM | 7231 | CG1 | VAL | F | 164 | −5.131 | −88.918 | 6.480 | 1.00 | 50.00 | C |
| ATOM | 7232 | CG2 | VAL | F | 164 | −4.641 | −86.775 | 5.135 | 1.00 | 50.00 | C |
| ATOM | 7233 | N | ASN | F | 165 | −5.697 | −86.621 | 8.711 | 1.00 | 50.00 | N |
| ATOM | 7234 | CA | ASN | F | 165 | −6.730 | −85.686 | 9.155 | 1.00 | 50.00 | C |
| ATOM | 7235 | C | ASN | F | 165 | −6.240 | −84.588 | 10.098 | 1.00 | 50.00 | C |
| ATOM | 7236 | O | ASN | F | 165 | −6.920 | −83.567 | 10.194 | 1.00 | 50.00 | O |
| ATOM | 7237 | CB | ASN | F | 165 | −7.916 | −86.587 | 9.498 | 1.00 | 50.00 | C |
| ATOM | 7238 | CG | ASN | F | 165 | −7.781 | −87.393 | 10.783 | 1.00 | 50.00 | C |
| ATOM | 7239 | OD1 | ASN | F | 165 | −7.328 | −86.894 | 11.813 | 1.00 | 50.00 | O |
| ATOM | 7240 | ND2 | ASN | F | 165 | −8.112 | −88.670 | 10.749 | 1.00 | 50.00 | N |
| ATOM | 7241 | N | LYS | F | 166 | −5.078 | −84.712 | 10.724 | 1.00 | 50.00 | N |
| ATOM | 7242 | CA | LYS | F | 166 | −4.489 | −83.794 | 11.701 | 1.00 | 50.00 | C |
| ATOM | 7243 | C | LYS | F | 166 | −4.150 | −82.388 | 11.217 | 1.00 | 50.00 | C |
| ATOM | 7244 | O | LYS | F | 166 | −5.049 | −81.552 | 11.182 | 1.00 | 50.00 | O |
| ATOM | 7245 | CB | LYS | F | 166 | −3.315 | −84.472 | 12.409 | 1.00 | 50.00 | C |
| ATOM | 7246 | CG | LYS | F | 166 | −3.724 | −85.529 | 13.431 | 1.00 | 50.00 | C |
| ATOM | 7247 | CD | LYS | F | 166 | −2.609 | −85.910 | 14.401 | 1.00 | 50.00 | C |
| ATOM | 7248 | CE | LYS | F | 166 | −3.104 | −86.979 | 15.374 | 1.00 | 50.00 | C |
| ATOM | 7249 | NZ | LYS | F | 166 | −2.033 | −87.394 | 16.292 | 1.00 | 50.00 | N |
| ATOM | 7250 | N | ILE | F | 167 | −2.936 | −82.004 | 10.859 | 1.00 | 50.00 | N |
| ATOM | 7251 | CA | ILE | F | 167 | −2.633 | −81.399 | 9.560 | 1.00 | 50.00 | C |
| ATOM | 7252 | C | ILE | F | 167 | −3.639 | −80.851 | 8.544 | 1.00 | 50.00 | C |
| ATOM | 7253 | O | ILE | F | 167 | −3.374 | −79.789 | 7.983 | 1.00 | 50.00 | O |
| ATOM | 7254 | CB | ILE | F | 167 | −1.701 | −82.422 | 8.915 | 1.00 | 50.00 | C |
| ATOM | 7255 | CG1 | ILE | F | 167 | −2.521 | −83.689 | 8.667 | 1.00 | 50.00 | C |
| ATOM | 7256 | CG2 | ILE | F | 167 | −0.233 | −82.736 | 9.196 | 1.00 | 50.00 | C |
| ATOM | 7257 | CD1 | ILE | F | 167 | −1.970 | −84.756 | 7.743 | 1.00 | 50.00 | C |
| ATOM | 7258 | N | LYS | F | 168 | −4.682 | −81.557 | 8.133 | 1.00 | 50.00 | N |
| ATOM | 7259 | CA | LYS | F | 168 | −5.788 | −81.123 | 7.277 | 1.00 | 50.00 | C |
| ATOM | 7260 | C | LYS | F | 168 | −6.661 | −80.142 | 8.053 | 1.00 | 50.00 | C |
| ATOM | 7261 | O | LYS | F | 168 | −6.825 | −79.007 | 7.609 | 1.00 | 50.00 | O |
| ATOM | 7262 | CB | LYS | F | 168 | −6.510 | −82.366 | 6.750 | 1.00 | 50.00 | C |
| ATOM | 7263 | CG | LYS | F | 168 | −7.743 | −82.141 | 5.876 | 1.00 | 50.00 | C |
| ATOM | 7264 | CD | LYS | F | 168 | −8.664 | −83.322 | 5.574 | 1.00 | 50.00 | C |
| ATOM | 7265 | CE | LYS | F | 168 | −9.969 | −82.839 | 4.944 | 1.00 | 50.00 | C |
| ATOM | 7266 | NZ | LYS | F | 168 | −10.875 | −82.113 | 5.847 | 1.00 | 50.00 | N |
| ATOM | 7267 | N | SER | F | 169 | −7.131 | −80.532 | 9.228 | 1.00 | 50.00 | N |
| ATOM | 7268 | CA | SER | F | 169 | −7.493 | −79.633 | 10.324 | 1.00 | 50.00 | C |
| ATOM | 7269 | C | SER | F | 169 | −6.507 | −78.541 | 10.728 | 1.00 | 50.00 | C |
| ATOM | 7270 | O | SER | F | 169 | −6.947 | −77.415 | 10.956 | 1.00 | 50.00 | O |
| ATOM | 7271 | CB | SER | F | 169 | −7.881 | −80.469 | 11.544 | 1.00 | 50.00 | C |
| ATOM | 7272 | OG | SER | F | 169 | −9.056 | −81.232 | 11.310 | 1.00 | 50.00 | O |
| ATOM | 7273 | N | ALA | F | 170 | −5.214 | −78.805 | 10.830 | 1.00 | 50.00 | N |
| ATOM | 7274 | CA | ALA | F | 170 | −4.193 | −77.799 | 11.122 | 1.00 | 50.00 | C |
| ATOM | 7275 | C | ALA | F | 170 | −3.931 | −76.812 | 9.990 | 1.00 | 50.00 | C |
| ATOM | 7276 | O | ALA | F | 170 | −3.769 | −75.627 | 10.277 | 1.00 | 50.00 | O |
| ATOM | 7277 | CB | ALA | F | 170 | −2.935 | −78.516 | 11.605 | 1.00 | 50.00 | C |
| ATOM | 7278 | N | LEU | F | 171 | −3.925 | −77.226 | 8.733 | 1.00 | 50.00 | N |
| ATOM | 7279 | CA | LEU | F | 171 | −3.968 | −76.340 | 7.571 | 1.00 | 50.00 | C |
| ATOM | 7280 | C | LEU | F | 171 | −5.225 | −75.482 | 7.527 | 1.00 | 50.00 | C |
| ATOM | 7281 | O | LEU | F | 171 | −5.116 | −74.272 | 7.343 | 1.00 | 50.00 | O |
| ATOM | 7282 | CB | LEU | F | 171 | −3.899 | −77.109 | 6.253 | 1.00 | 50.00 | C |
| ATOM | 7283 | CG | LEU | F | 171 | −3.430 | −76.278 | 5.059 | 1.00 | 50.00 | C |
| ATOM | 7284 | CD1 | LEU | F | 171 | −4.146 | −76.724 | 3.792 | 1.00 | 50.00 | C |
| ATOM | 7285 | CD2 | LEU | F | 171 | −3.601 | −74.775 | 4.884 | 1.00 | 50.00 | C |
| ATOM | 7286 | N | LEU | F | 172 | −6.405 | −76.048 | 7.718 | 1.00 | 50.00 | N |
| ATOM | 7287 | CA | LEU | F | 172 | −7.632 | −75.283 | 7.933 | 1.00 | 50.00 | C |
| ATOM | 7288 | C | LEU | F | 172 | −7.529 | −74.167 | 8.968 | 1.00 | 50.00 | C |
| ATOM | 7289 | O | LEU | F | 172 | −7.801 | −73.020 | 8.616 | 1.00 | 50.00 | O |
| ATOM | 7290 | CB | LEU | F | 172 | −8.751 | −76.292 | 8.197 | 1.00 | 50.00 | C |
| ATOM | 7291 | CG | LEU | F | 172 | −10.191 | −75.839 | 7.961 | 1.00 | 50.00 | C |
| ATOM | 7292 | CD1 | LEU | F | 172 | −10.655 | −74.863 | 9.038 | 1.00 | 50.00 | C |
| ATOM | 7293 | CD2 | LEU | F | 172 | −10.438 | −75.246 | 6.575 | 1.00 | 50.00 | C |
| ATOM | 7294 | N | SER | F | 173 | −7.075 | −74.453 | 10.178 | 1.00 | 50.00 | N |
| ATOM | 7295 | CA | SER | F | 173 | −6.693 | −73.451 | 11.176 | 1.00 | 50.00 | C |
| ATOM | 7296 | C | SER | F | 173 | −5.775 | −72.315 | 10.739 | 1.00 | 50.00 | C |
| ATOM | 7297 | O | SER | F | 173 | −5.962 | −71.159 | 11.115 | 1.00 | 50.00 | O |
| ATOM | 7298 | CB | SER | F | 173 | −6.058 | −74.156 | 12.375 | 1.00 | 50.00 | C |
| ATOM | 7299 | OG | SER | F | 173 | −7.067 | −74.728 | 13.193 | 1.00 | 50.00 | O |
| ATOM | 7300 | N | THR | F | 174 | −4.779 | −72.665 | 9.944 | 1.00 | 50.00 | N |
| ATOM | 7301 | CA | THR | F | 174 | −3.821 | −71.742 | 9.341 | 1.00 | 50.00 | C |
| ATOM | 7302 | C | THR | F | 174 | −4.331 | −70.967 | 8.129 | 1.00 | 50.00 | C |
| ATOM | 7303 | O | THR | F | 174 | −4.000 | −69.789 | 8.004 | 1.00 | 50.00 | O |
| ATOM | 7304 | CB | THR | F | 174 | −2.639 | −72.666 | 9.053 | 1.00 | 50.00 | C |
| ATOM | 7305 | OG1 | THR | F | 174 | −2.210 | −73.496 | 10.123 | 1.00 | 50.00 | O |
| ATOM | 7306 | CG2 | THR | F | 174 | −1.388 | −71.853 | 8.747 | 1.00 | 50.00 | C |
| ATOM | 7307 | N | ASN | F | 175 | −5.131 | −71.532 | 7.239 | 1.00 | 50.00 | N |
| ATOM | 7308 | CA | ASN | F | 175 | −5.963 | −70.803 | 6.280 | 1.00 | 50.00 | C |
| ATOM | 7309 | C | ASN | F | 175 | −6.802 | −69.671 | 6.863 | 1.00 | 50.00 | C |
| ATOM | 7310 | O | ASN | F | 175 | −6.705 | −68.546 | 6.376 | 1.00 | 50.00 | O |
| ATOM | 7311 | CB | ASN | F | 175 | −6.874 | −71.756 | 5.504 | 1.00 | 50.00 | C |
| ATOM | 7312 | CG | ASN | F | 175 | −6.276 | −72.544 | 4.348 | 1.00 | 50.00 | C |
| ATOM | 7313 | OD1 | ASN | F | 175 | −5.351 | −72.132 | 3.650 | 1.00 | 50.00 | O |
| ATOM | 7314 | ND2 | ASN | F | 175 | −6.804 | −73.734 | 4.128 | 1.00 | 50.00 | N |
| ATOM | 7315 | N | LYS | F | 176 | −7.544 | −69.949 | 7.923 | 1.00 | 50.00 | N |
| ATOM | 7316 | CA | LYS | F | 176 | −8.131 | −68.954 | 8.821 | 1.00 | 50.00 | C |
| ATOM | 7317 | C | LYS | F | 176 | −7.185 | −67.925 | 9.431 | 1.00 | 50.00 | C |
| ATOM | 7318 | O | LYS | F | 176 | −7.562 | −66.755 | 9.477 | 1.00 | 50.00 | O |
| ATOM | 7319 | CB | LYS | F | 176 | −8.867 | −69.669 | 9.955 | 1.00 | 50.00 | C |
| ATOM | 7320 | CG | LYS | F | 176 | −10.077 | −70.517 | 9.568 | 1.00 | 50.00 | C |
| ATOM | 7321 | CD | LYS | F | 176 | −10.772 | −71.077 | 10.808 | 1.00 | 50.00 | C |
| ATOM | 7322 | CE | LYS | F | 176 | −11.975 | −71.951 | 10.458 | 1.00 | 50.00 | C |
| ATOM | 7323 | NZ | LYS | F | 176 | −12.364 | −72.814 | 11.583 | 1.00 | 50.00 | N |
| ATOM | 7324 | N | ALA | F | 177 | −5.994 | −68.297 | 9.874 | 1.00 | 50.00 | N |
| ATOM | 7325 | CA | ALA | F | 177 | −4.926 | −67.364 | 10.236 | 1.00 | 50.00 | C |
| ATOM | 7326 | C | ALA | F | 177 | −4.506 | −66.343 | 9.183 | 1.00 | 50.00 | C |
| ATOM | 7327 | O | ALA | F | 177 | −4.265 | −65.187 | 9.530 | 1.00 | 50.00 | O |
| ATOM | 7328 | CB | ALA | F | 177 | −3.736 | −68.172 | 10.753 | 1.00 | 50.00 | C |
| ATOM | 7329 | N | VAL | F | 178 | −4.476 | −66.722 | 7.916 | 1.00 | 50.00 | N |
| ATOM | 7330 | CA | VAL | F | 178 | −4.281 | −65.827 | 6.774 | 1.00 | 50.00 | C |
| ATOM | 7331 | C | VAL | F | 178 | −5.519 | −64.986 | 6.478 | 1.00 | 50.00 | C |
| ATOM | 7332 | O | VAL | F | 178 | −5.311 | −63.797 | 6.244 | 1.00 | 50.00 | O |
| ATOM | 7333 | CB | VAL | F | 178 | −3.735 | −66.561 | 5.547 | 1.00 | 50.00 | C |
| ATOM | 7334 | CG1 | VAL | F | 178 | −3.509 | −65.614 | 4.366 | 1.00 | 50.00 | C |
| ATOM | 7335 | CG2 | VAL | F | 178 | −2.483 | −67.385 | 5.851 | 1.00 | 50.00 | C |
| ATOM | 7336 | N | VAL | F | 179 | −6.746 | −65.485 | 6.450 | 1.00 | 50.00 | N |
| ATOM | 7337 | CA | VAL | F | 179 | −7.970 | −64.681 | 6.379 | 1.00 | 50.00 | C |
| ATOM | 7338 | C | VAL | F | 179 | −8.026 | −63.574 | 7.427 | 1.00 | 50.00 | C |
| ATOM | 7339 | O | VAL | F | 179 | −8.197 | −62.415 | 7.053 | 1.00 | 50.00 | O |
| ATOM | 7340 | CB | VAL | F | 179 | −9.258 | −65.510 | 6.423 | 1.00 | 50.00 | C |
| ATOM | 7341 | CG1 | VAL | F | 179 | −10.578 | −64.740 | 6.381 | 1.00 | 50.00 | C |
| ATOM | 7342 | CG2 | VAL | F | 179 | −9.356 | −66.560 | 5.320 | 1.00 | 50.00 | C |
| ATOM | 7343 | N | SER | F | 180 | −7.882 | −63.914 | 8.697 | 1.00 | 50.00 | N |
| ATOM | 7344 | CA | SER | F | 180 | −7.787 | −62.976 | 9.816 | 1.00 | 50.00 | C |
| ATOM | 7345 | C | SER | F | 180 | −6.768 | −61.854 | 9.659 | 1.00 | 50.00 | C |
| ATOM | 7346 | O | SER | F | 180 | −7.138 | −60.687 | 9.787 | 1.00 | 50.00 | O |
| ATOM | 7347 | CB | SER | F | 180 | −7.518 | −63.768 | 11.095 | 1.00 | 50.00 | C |
| ATOM | 7348 | OG | SER | F | 180 | −8.633 | −64.576 | 11.444 | 1.00 | 50.00 | O |
| ATOM | 7349 | N | LEU | F | 181 | −5.528 | −62.185 | 9.338 | 1.00 | 50.00 | N |
| ATOM | 7350 | CA | LEU | F | 181 | −4.488 | −61.207 | 9.023 | 1.00 | 50.00 | C |
| ATOM | 7351 | C | LEU | F | 181 | −4.685 | −60.456 | 7.708 | 1.00 | 50.00 | C |
| ATOM | 7352 | O | LEU | F | 181 | −4.459 | −59.247 | 7.728 | 1.00 | 50.00 | O |
| ATOM | 7353 | CB | LEU | F | 181 | −3.158 | −61.910 | 9.302 | 1.00 | 50.00 | C |
| ATOM | 7354 | CG | LEU | F | 181 | −1.909 | −61.029 | 9.243 | 1.00 | 50.00 | C |
| ATOM | 7355 | CD1 | LEU | F | 181 | −1.859 | −60.027 | 10.394 | 1.00 | 50.00 | C |
| ATOM | 7356 | CD2 | LEU | F | 181 | −0.617 | −61.842 | 9.207 | 1.00 | 50.00 | C |
| ATOM | 7357 | N | SER | F | 182 | −5.164 | −61.033 | 6.617 | 1.00 | 50.00 | N |
| ATOM | 7358 | CA | SER | F | 182 | −5.688 | −60.330 | 5.443 | 1.00 | 50.00 | C |
| ATOM | 7359 | C | SER | F | 182 | −6.770 | −59.283 | 5.685 | 1.00 | 50.00 | C |
| ATOM | 7360 | O | SER | F | 182 | −6.655 | −58.180 | 5.153 | 1.00 | 50.00 | O |
| ATOM | 7361 | CB | SER | F | 182 | −6.164 | −61.314 | 4.374 | 1.00 | 50.00 | C |
| ATOM | 7362 | OG | SER | F | 182 | −5.122 | −62.141 | 3.876 | 1.00 | 50.00 | O |
| ATOM | 7363 | N | ASN | F | 183 | −7.776 | −59.587 | 6.489 | 1.00 | 50.00 | N |
| ATOM | 7364 | CA | ASN | F | 183 | −8.741 | −58.626 | 7.026 | 1.00 | 50.00 | C |
| ATOM | 7365 | C | ASN | F | 183 | −8.150 | −57.523 | 7.898 | 1.00 | 50.00 | C |
| ATOM | 7366 | O | ASN | F | 183 | −8.520 | −56.365 | 7.707 | 1.00 | 50.00 | O |
| ATOM | 7367 | CB | ASN | F | 183 | −9.855 | −59.363 | 7.771 | 1.00 | 50.00 | C |
| ATOM | 7368 | CG | ASN | F | 183 | −10.714 | −60.351 | 6.992 | 1.00 | 50.00 | C |
| ATOM | 7369 | OD1 | ASN | F | 183 | −10.953 | −60.216 | 5.793 | 1.00 | 50.00 | O |
| ATOM | 7370 | ND2 | ASN | F | 183 | −11.211 | −61.378 | 7.658 | 1.00 | 50.00 | N |
| ATOM | 7371 | N | GLY | F | 184 | −7.233 | −57.822 | 8.804 | 1.00 | 50.00 | N |
| ATOM | 7372 | CA | GLY | F | 184 | −6.401 | −56.836 | 9.495 | 1.00 | 50.00 | C |
| ATOM | 7373 | C | GLY | F | 184 | −5.571 | −55.871 | 8.658 | 1.00 | 50.00 | C |
| ATOM | 7374 | O | GLY | F | 184 | −5.616 | −54.670 | 8.922 | 1.00 | 50.00 | O |
| ATOM | 7375 | N | VAL | F | 185 | −4.869 | −56.360 | 7.648 | 1.00 | 50.00 | N |
| ATOM | 7376 | CA | VAL | F | 185 | −4.241 | −55.562 | 6.593 | 1.00 | 50.00 | C |
| ATOM | 7377 | C | VAL | F | 185 | −5.234 | −54.741 | 5.776 | 1.00 | 50.00 | C |
| ATOM | 7378 | O | VAL | F | 185 | −4.916 | −53.582 | 5.512 | 1.00 | 50.00 | O |
| ATOM | 7379 | CB | VAL | F | 185 | −3.308 | −56.397 | 5.710 | 1.00 | 50.00 | C |
| ATOM | 7380 | CG1 | VAL | F | 185 | −2.552 | −55.633 | 4.622 | 1.00 | 50.00 | C |
| ATOM | 7381 | CG2 | VAL | F | 185 | −2.213 | −57.133 | 6.481 | 1.00 | 50.00 | C |
| ATOM | 7382 | N | SER | F | 186 | −6.386 | −55.252 | 5.373 | 1.00 | 50.00 | N |
| ATOM | 7383 | CA | SER | F | 186 | −7.447 | −54.495 | 4.705 | 1.00 | 50.00 | C |
| ATOM | 7384 | C | SER | F | 186 | −7.985 | −53.276 | 5.449 | 1.00 | 50.00 | C |
| ATOM | 7385 | O | SER | F | 186 | −8.094 | −52.215 | 4.836 | 1.00 | 50.00 | O |
| ATOM | 7386 | CB | SER | F | 186 | −8.580 | −55.451 | 4.334 | 1.00 | 50.00 | C |
| ATOM | 7387 | OG | SER | F | 186 | −9.432 | −54.868 | 3.358 | 1.00 | 50.00 | O |
| ATOM | 7388 | N | VAL | F | 187 | −8.279 | −53.392 | 6.734 | 1.00 | 50.00 | N |
| ATOM | 7389 | CA | VAL | F | 187 | −8.575 | −52.272 | 7.630 | 1.00 | 50.00 | C |
| ATOM | 7390 | C | VAL | F | 187 | −7.398 | −51.318 | 7.809 | 1.00 | 50.00 | C |
| ATOM | 7391 | O | VAL | F | 187 | −7.611 | −50.109 | 7.741 | 1.00 | 50.00 | O |
| ATOM | 7392 | CB | VAL | F | 187 | −9.164 | −52.736 | 8.965 | 1.00 | 50.00 | C |
| ATOM | 7393 | CG1 | VAL | F | 187 | −9.515 | −51.600 | 9.927 | 1.00 | 50.00 | C |
| ATOM | 7394 | CG2 | VAL | F | 187 | −10.416 | −53.602 | 8.830 | 1.00 | 50.00 | C |
| ATOM | 7395 | N | LEU | F | 188 | −6.182 | −51.787 | 8.035 | 1.00 | 50.00 | N |
| ATOM | 7396 | CA | LEU | F | 188 | −4.979 | −50.954 | 8.035 | 1.00 | 50.00 | C |
| ATOM | 7397 | C | LEU | F | 188 | −4.668 | −50.197 | 6.747 | 1.00 | 50.00 | C |
| ATOM | 7398 | O | LEU | F | 188 | −4.261 | −49.039 | 6.825 | 1.00 | 50.00 | O |
| ATOM | 7399 | CB | LEU | F | 188 | −3.838 | −51.835 | 8.543 | 1.00 | 50.00 | C |
| ATOM | 7400 | CG | LEU | F | 188 | −2.529 | −51.137 | 8.914 | 1.00 | 50.00 | C |
| ATOM | 7401 | CD1 | LEU | F | 188 | −2.667 | −50.101 | 10.027 | 1.00 | 50.00 | C |
| ATOM | 7402 | CD2 | LEU | F | 188 | −1.466 | −52.174 | 9.272 | 1.00 | 50.00 | C |
| ATOM | 7403 | N | THR | F | 189 | −4.906 | −50.781 | 5.582 | 1.00 | 50.00 | N |
| ATOM | 7404 | CA | THR | F | 189 | −5.014 | −50.097 | 4.292 | 1.00 | 50.00 | C |
| ATOM | 7405 | C | THR | F | 189 | −6.121 | −49.049 | 4.270 | 1.00 | 50.00 | C |
| ATOM | 7406 | O | THR | F | 189 | −5.770 | −47.904 | 3.992 | 1.00 | 50.00 | O |
| ATOM | 7407 | CB | THR | F | 189 | −5.115 | −51.086 | 3.129 | 1.00 | 50.00 | C |
| ATOM | 7408 | OG1 | THR | F | 189 | −4.113 | −52.090 | 3.211 | 1.00 | 50.00 | O |
| ATOM | 7409 | CG2 | THR | F | 189 | −4.977 | −50.457 | 1.744 | 1.00 | 50.00 | C |
| ATOM | 7410 | N | SER | F | 190 | −7.374 | −49.346 | 4.579 | 1.00 | 50.00 | N |
| ATOM | 7411 | CA | SER | F | 190 | −8.431 | −48.361 | 4.824 | 1.00 | 50.00 | C |
| ATOM | 7412 | C | SER | F | 190 | −8.129 | −47.169 | 5.727 | 1.00 | 50.00 | C |
| ATOM | 7413 | O | SER | F | 190 | −8.620 | −46.066 | 5.488 | 1.00 | 50.00 | O |
| ATOM | 7414 | CB | SER | F | 190 | −9.742 | −49.062 | 5.180 | 1.00 | 50.00 | C |
| ATOM | 7415 | OG | SER | F | 190 | −9.908 | −49.302 | 6.570 | 1.00 | 50.00 | O |
| ATOM | 7416 | N | LYS | F | 191 | −7.300 | −47.367 | 6.738 | 1.00 | 50.00 | N |
| ATOM | 7417 | CA | LYS | F | 191 | −6.697 | −46.315 | 7.556 | 1.00 | 50.00 | C |
| ATOM | 7418 | C | LYS | F | 191 | −5.646 | −45.477 | 6.835 | 1.00 | 50.00 | C |
| ATOM | 7419 | O | LYS | F | 191 | −5.780 | −44.255 | 6.844 | 1.00 | 50.00 | O |
| ATOM | 7420 | CB | LYS | F | 191 | −6.288 | −46.955 | 8.884 | 1.00 | 50.00 | C |
| ATOM | 7421 | CG | LYS | F | 191 | −7.401 | −47.379 | 9.845 | 1.00 | 50.00 | C |
| ATOM | 7422 | CD | LYS | F | 191 | −8.582 | −46.433 | 10.067 | 1.00 | 50.00 | C |
| ATOM | 7423 | CE | LYS | F | 191 | −9.771 | −46.643 | 9.130 | 1.00 | 50.00 | C |
| ATOM | 7424 | NZ | LYS | F | 191 | −10.838 | −45.656 | 9.347 | 1.00 | 50.00 | N |
| ATOM | 7425 | N | VAL | F | 192 | −4.692 | −46.055 | 6.122 | 1.00 | 50.00 | N |
| ATOM | 7426 | CA | VAL | F | 192 | −3.988 | −45.438 | 4.994 | 1.00 | 50.00 | C |
| ATOM | 7427 | C | VAL | F | 192 | −4.832 | −44.674 | 3.972 | 1.00 | 50.00 | C |
| ATOM | 7428 | O | VAL | F | 192 | −4.414 | −43.564 | 3.646 | 1.00 | 50.00 | O |
| ATOM | 7429 | CB | VAL | F | 192 | −2.946 | −46.444 | 4.491 | 1.00 | 50.00 | C |
| ATOM | 7430 | CG1 | VAL | F | 192 | −2.749 | −46.553 | 2.980 | 1.00 | 50.00 | C |
| ATOM | 7431 | CG2 | VAL | F | 192 | −1.550 | −46.273 | 5.089 | 1.00 | 50.00 | C |
| ATOM | 7432 | N | LEU | F | 193 | −5.975 | −45.126 | 3.475 | 1.00 | 50.00 | N |
| ATOM | 7433 | CA | LEU | F | 193 | −6.914 | −44.346 | 2.663 | 1.00 | 50.00 | C |
| ATOM | 7434 | C | LEU | F | 193 | −7.371 | −43.040 | 3.308 | 1.00 | 50.00 | C |
| ATOM | 7435 | O | LEU | F | 193 | −7.234 | −41.986 | 2.687 | 1.00 | 50.00 | O |
| ATOM | 7436 | CB | LEU | F | 193 | −8.175 | −45.104 | 2.237 | 1.00 | 50.00 | C |
| ATOM | 7437 | CG | LEU | F | 193 | −8.218 | −46.166 | 1.138 | 1.00 | 50.00 | C |
| ATOM | 7438 | CD1 | LEU | F | 193 | −7.230 | −47.318 | 1.300 | 1.00 | 50.00 | C |
| ATOM | 7439 | CD2 | LEU | F | 193 | −9.622 | −46.763 | 1.038 | 1.00 | 50.00 | C |
| ATOM | 7440 | N | ASP | F | 194 | −7.931 | −43.097 | 4.506 | 1.00 | 50.00 | N |
| ATOM | 7441 | CA | ASP | F | 194 | −8.491 | −41.950 | 5.222 | 1.00 | 50.00 | C |
| ATOM | 7442 | C | ASP | F | 194 | −7.445 | −40.972 | 5.738 | 1.00 | 50.00 | C |
| ATOM | 7443 | O | ASP | F | 194 | −7.570 | −39.794 | 5.409 | 1.00 | 50.00 | O |
| ATOM | 7444 | CB | ASP | F | 194 | −9.367 | −42.360 | 6.406 | 1.00 | 50.00 | C |
| ATOM | 7445 | CG | ASP | F | 194 | −10.707 | −42.982 | 6.046 | 1.00 | 50.00 | C |
| ATOM | 7446 | OD1 | ASP | F | 194 | −11.516 | −42.320 | 5.361 | 1.00 | 50.00 | O |
| ATOM | 7447 | OD2 | ASP | F | 194 | −10.964 | −44.134 | 6.458 | 1.00 | 50.00 | O |
| ATOM | 7448 | N | LEU | F | 195 | −6.444 | −41.418 | 6.481 | 1.00 | 50.00 | N |
| ATOM | 7449 | CA | LEU | F | 195 | −5.267 | −40.620 | 6.822 | 1.00 | 50.00 | C |
| ATOM | 7450 | C | LEU | F | 195 | −4.725 | −39.795 | 5.663 | 1.00 | 50.00 | C |
| ATOM | 7451 | O | LEU | F | 195 | −4.733 | −38.568 | 5.744 | 1.00 | 50.00 | O |
| ATOM | 7452 | CB | LEU | F | 195 | −4.203 | −41.449 | 7.545 | 1.00 | 50.00 | C |
| ATOM | 7453 | CG | LEU | F | 195 | −3.075 | −40.683 | 8.237 | 1.00 | 50.00 | C |
| ATOM | 7454 | CD1 | LEU | F | 195 | −2.006 | −40.204 | 7.261 | 1.00 | 50.00 | C |
| ATOM | 7455 | CD2 | LEU | F | 195 | −3.498 | −39.426 | 8.992 | 1.00 | 50.00 | C |
| ATOM | 7456 | N | LYS | F | 196 | −4.347 | −40.453 | 4.581 | 1.00 | 50.00 | N |
| ATOM | 7457 | CA | LYS | F | 196 | −3.924 | −39.779 | 3.356 | 1.00 | 50.00 | C |
| ATOM | 7458 | C | LYS | F | 196 | −4.760 | −38.607 | 2.857 | 1.00 | 50.00 | C |
| ATOM | 7459 | O | LYS | F | 196 | −4.283 | −37.473 | 2.850 | 1.00 | 50.00 | O |
| ATOM | 7460 | CB | LYS | F | 196 | −3.771 | −40.867 | 2.292 | 1.00 | 50.00 | C |
| ATOM | 7461 | CG | LYS | F | 196 | −3.012 | −40.331 | 1.081 | 1.00 | 50.00 | C |
| ATOM | 7462 | CD | LYS | F | 196 | −1.551 | −40.330 | 1.519 | 1.00 | 50.00 | C |
| ATOM | 7463 | CE | LYS | F | 196 | −0.586 | −39.749 | 0.497 | 1.00 | 50.00 | C |
| ATOM | 7464 | NZ | LYS | F | 196 | 0.741 | −40.033 | 1.063 | 1.00 | 50.00 | N |
| ATOM | 7465 | N | ASN | F | 197 | −5.988 | −38.905 | 2.467 | 1.00 | 50.00 | N |
| ATOM | 7466 | CA | ASN | F | 197 | −6.976 | −37.918 | 2.036 | 1.00 | 50.00 | C |
| ATOM | 7467 | C | ASN | F | 197 | −7.286 | −36.827 | 3.054 | 1.00 | 50.00 | C |
| ATOM | 7468 | O | ASN | F | 197 | −7.370 | −35.673 | 2.638 | 1.00 | 50.00 | O |
| ATOM | 7469 | CB | ASN | F | 197 | −8.233 | −38.651 | 1.566 | 1.00 | 50.00 | C |
| ATOM | 7470 | CG | ASN | F | 197 | −8.148 | −39.538 | 0.330 | 1.00 | 50.00 | C |
| ATOM | 7471 | OD1 | ASN | F | 197 | −8.642 | −40.664 | 0.329 | 1.00 | 50.00 | O |
| ATOM | 7472 | ND2 | ASN | F | 197 | −7.557 | −39.080 | −0.759 | 1.00 | 50.00 | N |
| ATOM | 7473 | N | TYR | F | 198 | −7.350 | −37.121 | 4.344 | 1.00 | 50.00 | N |
| ATOM | 7474 | CA | TYR | F | 198 | −7.303 | −36.132 | 5.421 | 1.00 | 50.00 | C |
| ATOM | 7475 | C | TYR | F | 198 | −6.156 | −35.135 | 5.317 | 1.00 | 50.00 | C |
| ATOM | 7476 | O | TYR | F | 198 | −6.480 | −33.955 | 5.195 | 1.00 | 50.00 | O |
| ATOM | 7477 | CB | TYR | F | 198 | −7.346 | −36.789 | 6.802 | 1.00 | 50.00 | C |
| ATOM | 7478 | CG | TYR | F | 198 | −7.555 | −35.828 | 7.953 | 1.00 | 50.00 | C |
| ATOM | 7479 | CD1 | TYR | F | 198 | −8.809 | −35.467 | 8.295 | 1.00 | 50.00 | C |
| ATOM | 7480 | CD2 | TYR | F | 198 | −6.509 | −35.281 | 8.607 | 1.00 | 50.00 | C |
| ATOM | 7481 | CE1 | TYR | F | 198 | −9.018 | −34.627 | 9.328 | 1.00 | 50.00 | C |
| ATOM | 7482 | CE2 | TYR | F | 198 | −6.710 | −34.442 | 9.642 | 1.00 | 50.00 | C |
| ATOM | 7483 | CZ | TYR | F | 198 | −7.969 | −34.156 | 10.031 | 1.00 | 50.00 | C |
| ATOM | 7484 | OH | TYR | F | 198 | −8.196 | −33.407 | 11.156 | 1.00 | 50.00 | O |
| ATOM | 7485 | N | ILE | F | 199 | −4.894 | −35.534 | 5.346 | 1.00 | 50.00 | N |
| ATOM | 7486 | CA | ILE | F | 199 | −3.732 | −34.648 | 5.241 | 1.00 | 50.00 | C |
| ATOM | 7487 | C | ILE | F | 199 | −3.766 | −33.754 | 4.009 | 1.00 | 50.00 | C |
| ATOM | 7488 | O | ILE | F | 199 | −3.600 | −32.547 | 4.170 | 1.00 | 50.00 | O |
| ATOM | 7489 | CB | ILE | F | 199 | −2.418 | −35.414 | 5.423 | 1.00 | 50.00 | C |
| ATOM | 7490 | CG1 | ILE | F | 199 | −2.081 | −35.651 | 6.894 | 1.00 | 50.00 | C |
| ATOM | 7491 | CG2 | ILE | F | 199 | −1.115 | −34.806 | 4.910 | 1.00 | 50.00 | C |
| ATOM | 7492 | CD1 | ILE | F | 199 | −2.895 | −36.822 | 7.426 | 1.00 | 50.00 | C |
| ATOM | 7493 | N | ASP | F | 200 | −4.057 | −34.272 | 2.827 | 1.00 | 50.00 | N |
| ATOM | 7494 | CA | ASP | F | 200 | −4.427 | −33.478 | 1.655 | 1.00 | 50.00 | C |
| ATOM | 7495 | C | ASP | F | 200 | −5.489 | −32.422 | 1.948 | 1.00 | 50.00 | C |
| ATOM | 7496 | O | ASP | F | 200 | −5.133 | −31.247 | 2.027 | 1.00 | 50.00 | O |
| ATOM | 7497 | CB | ASP | F | 200 | −4.758 | −34.423 | 0.498 | 1.00 | 50.00 | C |
| ATOM | 7498 | CG | ASP | F | 200 | −3.647 | −35.358 | 0.042 | 1.00 | 50.00 | C |
| ATOM | 7499 | OD1 | ASP | F | 200 | −2.567 | −34.850 | −0.329 | 1.00 | 50.00 | O |
| ATOM | 7500 | OD2 | ASP | F | 200 | −3.828 | −36.595 | 0.063 | 1.00 | 50.00 | O |
| ATOM | 7501 | N | LYS | F | 201 | −6.714 | −32.780 | 2.297 | 1.00 | 50.00 | N |
| ATOM | 7502 | CA | LYS | F | 201 | −7.734 | −31.870 | 2.825 | 1.00 | 50.00 | C |
| ATOM | 7503 | C | LYS | F | 201 | −7.468 | −30.971 | 4.027 | 1.00 | 50.00 | C |
| ATOM | 7504 | O | LYS | F | 201 | −8.308 | −30.119 | 4.315 | 1.00 | 50.00 | O |
| ATOM | 7505 | CB | LYS | F | 201 | −8.957 | −32.658 | 3.292 | 1.00 | 50.00 | C |
| ATOM | 7506 | CG | LYS | F | 201 | −9.689 | −33.462 | 2.222 | 1.00 | 50.00 | C |
| ATOM | 7507 | CD | LYS | F | 201 | −10.886 | −34.149 | 2.873 | 1.00 | 50.00 | C |
| ATOM | 7508 | CE | LYS | F | 201 | −11.484 | −35.159 | 1.898 | 1.00 | 50.00 | C |
| ATOM | 7509 | NZ | LYS | F | 201 | −12.730 | −35.695 | 2.465 | 1.00 | 50.00 | N |
| ATOM | 7510 | N | GLN | F | 202 | −6.404 | −31.199 | 4.777 | 1.00 | 50.00 | N |
| ATOM | 7511 | CA | GLN | F | 202 | −6.002 | −30.418 | 5.946 | 1.00 | 50.00 | C |
| ATOM | 7512 | C | GLN | F | 202 | −4.788 | −29.509 | 5.814 | 1.00 | 50.00 | C |
| ATOM | 7513 | O | GLN | F | 202 | −4.696 | −28.487 | 6.493 | 1.00 | 50.00 | O |
| ATOM | 7514 | CB | GLN | F | 202 | −5.658 | −31.455 | 7.012 | 1.00 | 50.00 | C |
| ATOM | 7515 | CG | GLN | F | 202 | −6.069 | −30.968 | 8.395 | 1.00 | 50.00 | C |
| ATOM | 7516 | CD | GLN | F | 202 | −7.535 | −30.893 | 8.780 | 1.00 | 50.00 | C |
| ATOM | 7517 | OE1 | GLN | F | 202 | −8.481 | −30.937 | 7.995 | 1.00 | 50.00 | O |
| ATOM | 7518 | NE2 | GLN | F | 202 | −7.651 | −30.888 | 10.094 | 1.00 | 50.00 | N |
| ATOM | 7519 | N | LEU | F | 203 | −3.902 | −29.851 | 4.897 | 1.00 | 50.00 | N |
| ATOM | 7520 | CA | LEU | F | 203 | −2.871 | −28.977 | 4.345 | 1.00 | 50.00 | C |
| ATOM | 7521 | C | LEU | F | 203 | −3.430 | −28.032 | 3.290 | 1.00 | 50.00 | C |
| ATOM | 7522 | O | LEU | F | 203 | −3.050 | −26.872 | 3.418 | 1.00 | 50.00 | O |
| ATOM | 7523 | CB | LEU | F | 203 | −1.798 | −29.910 | 3.791 | 1.00 | 50.00 | C |
| ATOM | 7524 | CG | LEU | F | 203 | −0.924 | −30.637 | 4.815 | 1.00 | 50.00 | C |
| ATOM | 7525 | CD1 | LEU | F | 203 | −1.450 | −31.139 | 6.160 | 1.00 | 50.00 | C |
| ATOM | 7526 | CD2 | LEU | F | 203 | −0.446 | −31.853 | 4.029 | 1.00 | 50.00 | C |
| ATOM | 7527 | N | LEU | F | 204 | −4.323 | −28.391 | 2.381 | 1.00 | 50.00 | N |
| ATOM | 7528 | CA | LEU | F | 204 | −5.109 | −27.487 | 1.536 | 1.00 | 50.00 | C |
| ATOM | 7529 | C | LEU | F | 204 | −5.943 | −26.359 | 2.145 | 1.00 | 50.00 | C |
| ATOM | 7530 | O | LEU | F | 204 | −5.942 | −25.288 | 1.538 | 1.00 | 50.00 | O |
| ATOM | 7531 | CB | LEU | F | 204 | −5.960 | −28.374 | 0.625 | 1.00 | 50.00 | C |
| ATOM | 7532 | CG | LEU | F | 204 | −5.326 | −29.031 | −0.603 | 1.00 | 50.00 | C |
| ATOM | 7533 | CD1 | LEU | F | 204 | −4.007 | −29.791 | −0.465 | 1.00 | 50.00 | C |
| ATOM | 7534 | CD2 | LEU | F | 204 | −6.367 | −29.940 | −1.254 | 1.00 | 50.00 | C |
| ATOM | 7535 | N | PRO | F | 205 | −6.678 | −26.437 | 3.246 | 1.00 | 50.00 | N |
| ATOM | 7536 | CA | PRO | F | 205 | −7.110 | −25.311 | 4.073 | 1.00 | 50.00 | C |
| ATOM | 7537 | C | PRO | F | 205 | −5.919 | −24.530 | 4.617 | 1.00 | 50.00 | C |
| ATOM | 7538 | O | PRO | F | 205 | −5.831 | −23.386 | 4.176 | 1.00 | 50.00 | O |
| ATOM | 7539 | CB | PRO | F | 205 | −8.055 | −25.936 | 5.099 | 1.00 | 50.00 | C |
| ATOM | 7540 | CG | PRO | F | 205 | −7.531 | −27.344 | 5.357 | 1.00 | 50.00 | C |
| ATOM | 7541 | CD | PRO | F | 205 | −6.738 | −27.628 | 4.085 | 1.00 | 50.00 | C |
| ATOM | 7542 | N | ILE | F | 206 | −4.960 | −25.069 | 5.355 | 1.00 | 50.00 | N |
| ATOM | 7543 | CA | ILE | F | 206 | −3.662 | −24.447 | 5.634 | 1.00 | 50.00 | C |
| ATOM | 7544 | C | ILE | F | 206 | −2.987 | −23.736 | 4.462 | 1.00 | 50.00 | C |
| ATOM | 7545 | O | ILE | F | 206 | −2.490 | −22.631 | 4.675 | 1.00 | 50.00 | O |
| ATOM | 7546 | CB | ILE | F | 206 | −2.707 | −25.345 | 6.428 | 1.00 | 50.00 | C |
| ATOM | 7547 | CG1 | ILE | F | 206 | −3.262 | −25.963 | 7.714 | 1.00 | 50.00 | C |
| ATOM | 7548 | CG2 | ILE | F | 206 | −1.407 | −24.624 | 6.787 | 1.00 | 50.00 | C |
| ATOM | 7549 | CD1 | ILE | F | 206 | −2.583 | −27.231 | 8.229 | 1.00 | 50.00 | C |
| ATOM | 7550 | N | VAL | F | 207 | −2.969 | −24.277 | 3.254 | 1.00 | 50.00 | N |
| ATOM | 7551 | CA | VAL | F | 207 | −2.487 | −23.682 | 2.007 | 1.00 | 50.00 | C |
| ATOM | 7552 | C | VAL | F | 207 | −3.114 | −22.307 | 1.783 | 1.00 | 50.00 | C |
| ATOM | 7553 | O | VAL | F | 207 | −2.385 | −21.316 | 1.805 | 1.00 | 50.00 | O |
| ATOM | 7554 | CB | VAL | F | 207 | −2.651 | −24.684 | 0.858 | 1.00 | 50.00 | C |
| ATOM | 7555 | CG1 | VAL | F | 207 | −2.543 | −24.089 | −0.544 | 1.00 | 50.00 | C |
| ATOM | 7556 | CG2 | VAL | F | 207 | −1.721 | −25.894 | 0.759 | 1.00 | 50.00 | C |
| ATOM | 7557 | N | ASN | F | 208 | −4.423 | −22.230 | 1.601 | 1.00 | 50.00 | N |
| ATOM | 7558 | CA | ASN | F | 208 | −5.161 | −20.977 | 1.428 | 1.00 | 50.00 | C |
| ATOM | 7559 | C | ASN | F | 208 | −5.496 | −20.158 | 2.671 | 1.00 | 50.00 | C |
| ATOM | 7560 | O | ASN | F | 208 | −5.678 | −18.948 | 2.542 | 1.00 | 50.00 | O |
| ATOM | 7561 | CB | ASN | F | 208 | −6.418 | −21.238 | 0.597 | 1.00 | 50.00 | C |
| ATOM | 7562 | CG | ASN | F | 208 | −6.261 | −21.639 | −0.864 | 1.00 | 50.00 | C |
| ATOM | 7563 | OD1 | ASN | F | 208 | −6.904 | −22.577 | −1.332 | 1.00 | 50.00 | O |
| ATOM | 7564 | ND2 | ASN | F | 208 | −5.449 | −20.945 | −1.641 | 1.00 | 50.00 | N |
| ATOM | 7565 | N | LYS | F | 209 | −5.550 | −20.724 | 3.866 | 1.00 | 50.00 | N |
| ATOM | 7566 | CA | LYS | F | 209 | −5.434 | −20.017 | 5.143 | 1.00 | 50.00 | C |
| ATOM | 7567 | C | LYS | F | 209 | −4.164 | −19.184 | 5.282 | 1.00 | 50.00 | C |
| ATOM | 7568 | O | LYS | F | 209 | −4.259 | −18.022 | 5.675 | 1.00 | 50.00 | O |
| ATOM | 7569 | CB | LYS | F | 209 | −5.567 | −20.982 | 6.323 | 1.00 | 50.00 | C |
| ATOM | 7570 | CG | LYS | F | 209 | −6.939 | −21.625 | 6.524 | 1.00 | 50.00 | C |
| ATOM | 7571 | CD | LYS | F | 209 | −6.977 | −22.691 | 7.618 | 1.00 | 50.00 | C |
| ATOM | 7572 | CE | LYS | F | 209 | −8.361 | −23.327 | 7.729 | 1.00 | 50.00 | C |
| ATOM | 7573 | NZ | LYS | F | 209 | −8.352 | −24.518 | 8.593 | 1.00 | 50.00 | N |
| ATOM | 7574 | N | GLN | F | 210 | −3.015 | −19.713 | 4.893 | 1.00 | 50.00 | N |
| ATOM | 7575 | CA | GLN | F | 210 | −1.804 | −18.941 | 4.612 | 1.00 | 50.00 | C |
| ATOM | 7576 | C | GLN | F | 210 | −1.935 | −17.839 | 3.565 | 1.00 | 50.00 | C |
| ATOM | 7577 | O | GLN | F | 210 | −1.386 | −16.766 | 3.812 | 1.00 | 50.00 | O |
| ATOM | 7578 | CB | GLN | F | 210 | −0.654 | −19.903 | 4.307 | 1.00 | 50.00 | C |
| ATOM | 7579 | CG | GLN | F | 210 | 0.665 | −19.614 | 5.021 | 1.00 | 50.00 | C |
| ATOM | 7580 | CD | GLN | F | 210 | 1.387 | −18.370 | 4.525 | 1.00 | 50.00 | C |
| ATOM | 7581 | OE1 | GLN | F | 210 | 1.413 | −18.056 | 3.337 | 1.00 | 50.00 | O |
| ATOM | 7582 | NE2 | GLN | F | 210 | 1.982 | −17.626 | 5.437 | 1.00 | 50.00 | N |
| ATOM | 7583 | N | SER | F | 211 | −2.644 | −18.030 | 2.463 | 1.00 | 50.00 | N |
| ATOM | 7584 | CA | SER | F | 211 | −2.951 | −16.982 | 1.486 | 1.00 | 50.00 | C |
| ATOM | 7585 | C | SER | F | 211 | −3.773 | −15.808 | 2.005 | 1.00 | 50.00 | C |
| ATOM | 7586 | O | SER | F | 211 | −3.352 | −14.667 | 1.819 | 1.00 | 50.00 | O |
| ATOM | 7587 | CB | SER | F | 211 | −3.620 | −17.544 | 0.232 | 1.00 | 50.00 | C |
| ATOM | 7588 | OG | SER | F | 211 | −2.799 | −18.490 | −0.438 | 1.00 | 50.00 | O |
| ATOM | 7589 | N | CYS | F | 212 | −4.900 | −16.028 | 2.663 | 1.00 | 50.00 | N |
| ATOM | 7590 | CA | CYS | F | 212 | −5.686 | −14.998 | 3.342 | 1.00 | 50.00 | C |
| ATOM | 7591 | C | CYS | F | 212 | −4.923 | −14.060 | 4.270 | 1.00 | 50.00 | C |
| ATOM | 7592 | O | CYS | F | 212 | −5.024 | −12.845 | 4.105 | 1.00 | 50.00 | O |
| ATOM | 7593 | CB | CYS | F | 212 | −6.845 | −15.707 | 4.043 | 1.00 | 50.00 | C |
| ATOM | 7594 | SG | CYS | F | 212 | −8.329 | −14.691 | 4.102 | 1.00 | 50.00 | S |
| ATOM | 7595 | N | SER | F | 213 | −4.090 | −14.599 | 5.146 | 1.00 | 50.00 | N |
| ATOM | 7596 | CA | SER | F | 213 | −3.044 | −13.867 | 5.860 | 1.00 | 50.00 | C |
| ATOM | 7597 | C | SER | F | 213 | −2.002 | −13.173 | 4.990 | 1.00 | 50.00 | C |
| ATOM | 7598 | O | SER | F | 213 | −1.971 | −11.947 | 5.083 | 1.00 | 50.00 | O |
| ATOM | 7599 | CB | SER | F | 213 | −2.443 | −14.783 | 6.923 | 1.00 | 50.00 | C |
| ATOM | 7600 | OG | SER | F | 213 | −3.390 | −14.989 | 7.962 | 1.00 | 50.00 | O |
| ATOM | 7601 | N | ILE | F | 214 | −1.228 | −13.826 | 4.134 | 1.00 | 50.00 | N |
| ATOM | 7602 | CA | ILE | F | 214 | −0.357 | −13.207 | 3.128 | 1.00 | 50.00 | C |
| ATOM | 7603 | C | ILE | F | 214 | −0.870 | −11.948 | 2.433 | 1.00 | 50.00 | C |
| ATOM | 7604 | O | ILE | F | 214 | −0.177 | −10.935 | 2.344 | 1.00 | 50.00 | O |
| ATOM | 7605 | CB | ILE | F | 214 | 0.264 | −14.234 | 2.172 | 1.00 | 50.00 | C |
| ATOM | 7606 | CG1 | ILE | F | 214 | 1.794 | −14.188 | 2.166 | 1.00 | 50.00 | C |
| ATOM | 7607 | CG2 | ILE | F | 214 | −0.234 | −14.245 | 0.725 | 1.00 | 50.00 | C |
| ATOM | 7608 | CD1 | ILE | F | 214 | 2.520 | −15.345 | 1.481 | 1.00 | 50.00 | C |
| ATOM | 7609 | N | SER | F | 215 | −2.095 | −12.048 | 1.942 | 1.00 | 50.00 | N |
| ATOM | 7610 | CA | SER | F | 215 | −2.845 | −10.989 | 1.270 | 1.00 | 50.00 | C |
| ATOM | 7611 | C | SER | F | 215 | −3.305 | −9.891 | 2.219 | 1.00 | 50.00 | C |
| ATOM | 7612 | O | SER | F | 215 | −3.063 | −8.727 | 1.902 | 1.00 | 50.00 | O |
| ATOM | 7613 | CB | SER | F | 215 | −4.053 | −11.606 | 0.565 | 1.00 | 50.00 | C |
| ATOM | 7614 | OG | SER | F | 215 | −3.693 | −12.649 | −0.328 | 1.00 | 50.00 | O |
| ATOM | 7615 | N | ASN | F | 216 | −3.893 | −10.207 | 3.363 | 1.00 | 50.00 | N |
| ATOM | 7616 | CA | ASN | F | 216 | −4.184 | −9.244 | 4.425 | 1.00 | 50.00 | C |
| ATOM | 7617 | C | ASN | F | 216 | −2.978 | −8.427 | 4.857 | 1.00 | 50.00 | C |
| ATOM | 7618 | O | ASN | F | 216 | −2.981 | −7.241 | 4.533 | 1.00 | 50.00 | O |
| ATOM | 7619 | CB | ASN | F | 216 | −4.952 | −9.821 | 5.616 | 1.00 | 50.00 | C |
| ATOM | 7620 | CG | ASN | F | 216 | −6.359 | −10.329 | 5.341 | 1.00 | 50.00 | C |
| ATOM | 7621 | OD1 | ASN | F | 216 | −6.850 | −11.319 | 5.881 | 1.00 | 50.00 | O |
| ATOM | 7622 | ND2 | ASN | F | 216 | −7.020 | −9.623 | 4.447 | 1.00 | 50.00 | N |
| ATOM | 7623 | N | ILE | F | 217 | −1.950 | −9.049 | 5.412 | 1.00 | 50.00 | N |
| ATOM | 7624 | CA | ILE | F | 217 | −0.594 | −8.508 | 5.521 | 1.00 | 50.00 | C |
| ATOM | 7625 | C | ILE | F | 217 | −0.162 | −7.510 | 4.451 | 1.00 | 50.00 | C |
| ATOM | 7626 | O | ILE | F | 217 | 0.537 | −6.581 | 4.847 | 1.00 | 50.00 | O |
| ATOM | 7627 | CB | ILE | F | 217 | 0.449 | −9.613 | 5.710 | 1.00 | 50.00 | C |
| ATOM | 7628 | CG1 | ILE | F | 217 | 0.202 | −10.596 | 6.856 | 1.00 | 50.00 | C |
| ATOM | 7629 | CG2 | ILE | F | 217 | 1.860 | −9.064 | 5.925 | 1.00 | 50.00 | C |
| ATOM | 7630 | CD1 | ILE | F | 217 | 0.975 | −11.910 | 6.762 | 1.00 | 50.00 | C |
| ATOM | 7631 | N | GLU | F | 218 | −0.528 | −7.611 | 3.183 | 1.00 | 50.00 | N |
| ATOM | 7632 | CA | GLU | F | 218 | −0.325 | −6.554 | 2.192 | 1.00 | 50.00 | C |
| ATOM | 7633 | C | GLU | F | 218 | −1.295 | −5.387 | 2.091 | 1.00 | 50.00 | C |
| ATOM | 7634 | O | GLU | F | 218 | −0.888 | −4.226 | 2.065 | 1.00 | 50.00 | O |
| ATOM | 7635 | CB | GLU | F | 218 | −0.220 | −7.179 | 0.801 | 1.00 | 50.00 | C |
| ATOM | 7636 | CG | GLU | F | 218 | 1.195 | −7.685 | 0.539 | 1.00 | 50.00 | C |
| ATOM | 7637 | CD | GLU | F | 218 | 2.121 | −6.493 | 0.345 | 1.00 | 50.00 | C |
| ATOM | 7638 | OE1 | GLU | F | 218 | 2.555 | −5.887 | 1.348 | 1.00 | 50.00 | O |
| ATOM | 7639 | OE2 | GLU | F | 218 | 2.408 | −6.129 | −0.815 | 1.00 | 50.00 | O |
| ATOM | 7640 | N | THR | F | 219 | −2.558 | −5.737 | 1.929 | 1.00 | 50.00 | N |
| ATOM | 7641 | CA | THR | F | 219 | −3.688 | −4.817 | 1.821 | 1.00 | 50.00 | C |
| ATOM | 7642 | C | THR | F | 219 | −3.739 | −3.849 | 2.997 | 1.00 | 50.00 | C |
| ATOM | 7643 | O | THR | F | 219 | −3.664 | −2.640 | 2.786 | 1.00 | 50.00 | O |
| ATOM | 7644 | CB | THR | F | 219 | −4.936 | −5.677 | 1.619 | 1.00 | 50.00 | C |
| ATOM | 7645 | OG1 | THR | F | 219 | −5.295 | −6.420 | 2.776 | 1.00 | 50.00 | O |
| ATOM | 7646 | CG2 | THR | F | 219 | −4.884 | −6.631 | 0.426 | 1.00 | 50.00 | C |
| ATOM | 7647 | N | VAL | F | 220 | −3.711 | −4.397 | 4.200 | 1.00 | 50.00 | N |
| ATOM | 7648 | CA | VAL | F | 220 | −3.343 | −3.730 | 5.446 | 1.00 | 50.00 | C |
| ATOM | 7649 | C | VAL | F | 220 | −2.196 | −2.732 | 5.528 | 1.00 | 50.00 | C |
| ATOM | 7650 | O | VAL | F | 220 | −2.192 | −1.890 | 6.423 | 1.00 | 50.00 | O |
| ATOM | 7651 | CB | VAL | F | 220 | −3.085 | −4.766 | 6.540 | 1.00 | 50.00 | C |
| ATOM | 7652 | CG1 | VAL | F | 220 | −4.119 | −5.843 | 6.836 | 1.00 | 50.00 | C |
| ATOM | 7653 | CG2 | VAL | F | 220 | −1.748 | −5.450 | 6.803 | 1.00 | 50.00 | C |
| ATOM | 7654 | N | ILE | F | 221 | −1.135 | −2.878 | 4.755 | 1.00 | 50.00 | N |
| ATOM | 7655 | CA | ILE | F | 221 | −0.101 | −1.849 | 4.643 | 1.00 | 50.00 | C |
| ATOM | 7656 | C | ILE | F | 221 | −0.546 | −0.832 | 3.599 | 1.00 | 50.00 | C |
| ATOM | 7657 | O | ILE | F | 221 | −0.400 | 0.365 | 3.843 | 1.00 | 50.00 | O |
| ATOM | 7658 | CB | ILE | F | 221 | 1.312 | −2.429 | 4.544 | 1.00 | 50.00 | C |
| ATOM | 7659 | CG1 | ILE | F | 221 | 1.861 | −2.980 | 5.861 | 1.00 | 50.00 | C |
| ATOM | 7660 | CG2 | ILE | F | 221 | 2.415 | −1.468 | 4.108 | 1.00 | 50.00 | C |
| ATOM | 7661 | CD1 | ILE | F | 221 | 0.907 | −3.969 | 6.517 | 1.00 | 50.00 | C |
| ATOM | 7662 | N | GLU | F | 222 | −1.062 | −1.324 | 2.484 | 1.00 | 50.00 | N |
| ATOM | 7663 | CA | GLU | F | 222 | −1.530 | −0.562 | 1.328 | 1.00 | 50.00 | C |
| ATOM | 7664 | C | GLU | F | 222 | −2.868 | 0.150 | 1.487 | 1.00 | 50.00 | C |
| ATOM | 7665 | O | GLU | F | 222 | −3.460 | 0.730 | 0.581 | 1.00 | 50.00 | O |
| ATOM | 7666 | CB | GLU | F | 222 | −1.448 | −1.491 | 0.115 | 1.00 | 50.00 | C |
| ATOM | 7667 | CG | GLU | F | 222 | −1.109 | −0.918 | −1.261 | 1.00 | 50.00 | C |
| ATOM | 7668 | CD | GLU | F | 222 | −1.857 | 0.351 | −1.633 | 1.00 | 50.00 | C |
| ATOM | 7669 | OE1 | GLU | F | 222 | −1.612 | 1.337 | −0.911 | 1.00 | 50.00 | O |
| ATOM | 7670 | OE2 | GLU | F | 222 | −2.745 | 0.448 | −2.503 | 1.00 | 50.00 | O |
| ATOM | 7671 | N | PHE | F | 223 | −3.305 | 0.349 | 2.714 | 1.00 | 50.00 | N |
| ATOM | 7672 | CA | PHE | F | 223 | −4.452 | 1.160 | 3.115 | 1.00 | 50.00 | C |
| ATOM | 7673 | C | PHE | F | 223 | −4.001 | 2.061 | 4.272 | 1.00 | 50.00 | C |
| ATOM | 7674 | O | PHE | F | 223 | −4.558 | 3.150 | 4.407 | 1.00 | 50.00 | O |
| ATOM | 7675 | CB | PHE | F | 223 | −5.465 | 0.030 | 3.326 | 1.00 | 50.00 | C |
| ATOM | 7676 | CG | PHE | F | 223 | −6.579 | −0.768 | 2.678 | 1.00 | 50.00 | C |
| ATOM | 7677 | CD1 | PHE | F | 223 | −7.685 | −0.188 | 2.167 | 1.00 | 50.00 | C |
| ATOM | 7678 | CD2 | PHE | F | 223 | −6.617 | −2.102 | 2.886 | 1.00 | 50.00 | C |
| ATOM | 7679 | CE1 | PHE | F | 223 | −8.683 | −0.939 | 1.660 | 1.00 | 50.00 | C |
| ATOM | 7680 | CE2 | PHE | F | 223 | −7.568 | −2.875 | 2.326 | 1.00 | 50.00 | C |
| ATOM | 7681 | CZ | PHE | F | 223 | −8.573 | −2.283 | 1.651 | 1.00 | 50.00 | C |
| ATOM | 7682 | N | GLN | F | 224 | −3.020 | 1.736 | 5.105 | 1.00 | 50.00 | N |
| ATOM | 7683 | CA | GLN | F | 224 | −2.401 | 2.498 | 6.193 | 1.00 | 50.00 | C |
| ATOM | 7684 | C | GLN | F | 224 | −1.458 | 3.653 | 5.895 | 1.00 | 50.00 | C |
| ATOM | 7685 | O | GLN | F | 224 | −1.713 | 4.790 | 6.292 | 1.00 | 50.00 | O |
| ATOM | 7686 | CB | GLN | F | 224 | −1.556 | 1.544 | 7.037 | 1.00 | 50.00 | C |
| ATOM | 7687 | CG | GLN | F | 224 | −2.080 | 0.541 | 8.054 | 1.00 | 50.00 | C |
| ATOM | 7688 | CD | GLN | F | 224 | −2.681 | 1.266 | 9.241 | 1.00 | 50.00 | C |
| ATOM | 7689 | OE1 | GLN | F | 224 | −2.566 | 2.461 | 9.513 | 1.00 | 50.00 | O |
| ATOM | 7690 | NE2 | GLN | F | 224 | −3.457 | 0.417 | 9.881 | 1.00 | 50.00 | N |
| ATOM | 7691 | N | GLN | F | 225 | −0.375 | 3.333 | 5.208 | 1.00 | 50.00 | N |
| ATOM | 7692 | CA | GLN | F | 225 | 0.390 | 4.278 | 4.401 | 1.00 | 50.00 | C |
| ATOM | 7693 | C | GLN | F | 225 | −0.472 | 5.272 | 3.616 | 1.00 | 50.00 | C |
| ATOM | 7694 | O | GLN | F | 225 | 0.048 | 6.280 | 3.143 | 1.00 | 50.00 | O |
| ATOM | 7695 | CB | GLN | F | 225 | 1.478 | 3.423 | 3.747 | 1.00 | 50.00 | C |
| ATOM | 7696 | CG | GLN | F | 225 | 2.586 | 2.546 | 4.310 | 1.00 | 50.00 | C |
| ATOM | 7697 | CD | GLN | F | 225 | 3.662 | 3.282 | 5.092 | 1.00 | 50.00 | C |
| ATOM | 7698 | OE1 | GLN | F | 225 | 4.107 | 4.381 | 4.764 | 1.00 | 50.00 | O |
| ATOM | 7699 | NE2 | GLN | F | 225 | 4.163 | 2.607 | 6.107 | 1.00 | 50.00 | N |
| ATOM | 7700 | N | LYS | F | 226 | −1.722 | 5.032 | 3.249 | 1.00 | 50.00 | N |
| ATOM | 7701 | CA | LYS | F | 226 | −2.571 | 5.887 | 2.417 | 1.00 | 50.00 | C |
| ATOM | 7702 | C | LYS | F | 226 | −3.163 | 6.942 | 3.336 | 1.00 | 50.00 | C |
| ATOM | 7703 | O | LYS | F | 226 | −3.005 | 8.115 | 3.002 | 1.00 | 50.00 | O |
| ATOM | 7704 | CB | LYS | F | 226 | −3.690 | 5.191 | 1.639 | 1.00 | 50.00 | C |
| ATOM | 7705 | CG | LYS | F | 226 | −4.600 | 6.094 | 0.802 | 1.00 | 50.00 | C |
| ATOM | 7706 | CD | LYS | F | 226 | −3.938 | 6.564 | −0.491 | 1.00 | 50.00 | C |
| ATOM | 7707 | CE | LYS | F | 226 | −4.544 | 7.745 | −1.244 | 1.00 | 50.00 | C |
| ATOM | 7708 | NZ | LYS | F | 226 | −3.835 | 7.985 | −2.508 | 1.00 | 50.00 | N |
| ATOM | 7709 | N | ASN | F | 227 | −3.793 | 6.549 | 4.430 | 1.00 | 50.00 | N |
| ATOM | 7710 | CA | ASN | F | 227 | −4.255 | 7.466 | 5.468 | 1.00 | 50.00 | C |
| ATOM | 7711 | C | ASN | F | 227 | −3.216 | 8.503 | 5.878 | 1.00 | 50.00 | C |
| ATOM | 7712 | O | ASN | F | 227 | −3.404 | 9.646 | 5.466 | 1.00 | 50.00 | O |
| ATOM | 7713 | CB | ASN | F | 227 | −4.842 | 6.610 | 6.588 | 1.00 | 50.00 | C |
| ATOM | 7714 | CG | ASN | F | 227 | −5.714 | 7.504 | 7.456 | 1.00 | 50.00 | C |
| ATOM | 7715 | OD1 | ASN | F | 227 | −6.902 | 7.753 | 7.263 | 1.00 | 50.00 | O |
| ATOM | 7716 | ND2 | ASN | F | 227 | −5.046 | 8.187 | 8.363 | 1.00 | 50.00 | N |
| ATOM | 7717 | N | ASN | F | 228 | −2.109 | 8.178 | 6.526 | 1.00 | 50.00 | N |
| ATOM | 7718 | CA | ASN | F | 228 | −0.955 | 9.073 | 6.619 | 1.00 | 50.00 | C |
| ATOM | 7719 | C | ASN | F | 228 | −0.582 | 9.915 | 5.402 | 1.00 | 50.00 | C |
| ATOM | 7720 | O | ASN | F | 228 | −0.318 | 11.103 | 5.573 | 1.00 | 50.00 | O |
| ATOM | 7721 | CB | ASN | F | 228 | 0.223 | 8.243 | 7.125 | 1.00 | 50.00 | C |
| ATOM | 7722 | CG | ASN | F | 228 | 0.743 | 7.104 | 6.266 | 1.00 | 50.00 | C |
| ATOM | 7723 | OD1 | ASN | F | 228 | 0.773 | 5.993 | 6.791 | 1.00 | 50.00 | O |
| ATOM | 7724 | ND2 | ASN | F | 228 | 1.302 | 7.404 | 5.106 | 1.00 | 50.00 | N |
| ATOM | 7725 | N | ARG | F | 229 | −0.611 | 9.373 | 4.195 | 1.00 | 50.00 | N |
| ATOM | 7726 | CA | ARG | F | 229 | −0.252 | 10.067 | 2.959 | 1.00 | 50.00 | C |
| ATOM | 7727 | C | ARG | F | 229 | −1.225 | 11.204 | 2.680 | 1.00 | 50.00 | C |
| ATOM | 7728 | O | ARG | F | 229 | −0.855 | 12.365 | 2.839 | 1.00 | 50.00 | O |
| ATOM | 7729 | CB | ARG | F | 229 | −0.119 | 9.093 | 1.788 | 1.00 | 50.00 | C |
| ATOM | 7730 | CG | ARG | F | 229 | 0.595 | 9.633 | 0.550 | 1.00 | 50.00 | C |
| ATOM | 7731 | CD | ARG | F | 229 | 0.270 | 9.038 | −0.815 | 1.00 | 50.00 | C |
| ATOM | 7732 | NE | ARG | F | 229 | 0.319 | 7.581 | −0.746 | 1.00 | 50.00 | N |
| ATOM | 7733 | CZ | ARG | F | 229 | −0.502 | 6.843 | −1.469 | 1.00 | 50.00 | C |
| ATOM | 7734 | NH1 | ARG | F | 229 | −0.990 | 7.137 | −2.657 | 1.00 | 50.00 | N |
| ATOM | 7735 | NH2 | ARG | F | 229 | −0.908 | 5.710 | −0.943 | 1.00 | 50.00 | N |
| ATOM | 7736 | N | LEU | F | 230 | −2.481 | 10.871 | 2.443 | 1.00 | 50.00 | N |
| ATOM | 7737 | CA | LEU | F | 230 | −3.646 | 11.729 | 2.654 | 1.00 | 50.00 | C |
| ATOM | 7738 | C | LEU | F | 230 | −3.631 | 12.797 | 3.739 | 1.00 | 50.00 | C |
| ATOM | 7739 | O | LEU | F | 230 | −3.910 | 13.960 | 3.454 | 1.00 | 50.00 | O |
| ATOM | 7740 | CB | LEU | F | 230 | −4.805 | 10.769 | 2.924 | 1.00 | 50.00 | C |
| ATOM | 7741 | CG | LEU | F | 230 | −6.050 | 11.002 | 2.074 | 1.00 | 50.00 | C |
| ATOM | 7742 | CD1 | LEU | F | 230 | −5.782 | 10.630 | 0.617 | 1.00 | 50.00 | C |
| ATOM | 7743 | CD2 | LEU | F | 230 | −7.098 | 10.058 | 2.658 | 1.00 | 50.00 | C |
| ATOM | 7744 | N | LEU | F | 231 | −3.306 | 12.405 | 4.960 | 1.00 | 50.00 | N |
| ATOM | 7745 | CA | LEU | F | 231 | −3.193 | 13.300 | 6.109 | 1.00 | 50.00 | C |
| ATOM | 7746 | C | LEU | F | 231 | −2.015 | 14.244 | 5.909 | 1.00 | 50.00 | C |
| ATOM | 7747 | O | LEU | F | 231 | −2.350 | 15.409 | 5.710 | 1.00 | 50.00 | O |
| ATOM | 7748 | CB | LEU | F | 231 | −3.170 | 12.503 | 7.411 | 1.00 | 50.00 | C |
| ATOM | 7749 | CG | LEU | F | 231 | −4.527 | 12.012 | 7.914 | 1.00 | 50.00 | C |
| ATOM | 7750 | CD1 | LEU | F | 231 | −5.485 | 11.201 | 7.042 | 1.00 | 50.00 | C |
| ATOM | 7751 | CD2 | LEU | F | 231 | −4.257 | 11.249 | 9.208 | 1.00 | 50.00 | C |
| ATOM | 7752 | N | GLU | F | 232 | −0.768 | 13.822 | 5.769 | 1.00 | 50.00 | N |
| ATOM | 7753 | CA | GLU | F | 232 | 0.337 | 14.618 | 5.230 | 1.00 | 50.00 | C |
| ATOM | 7754 | C | GLU | F | 232 | −0.055 | 15.646 | 4.179 | 1.00 | 50.00 | C |
| ATOM | 7755 | O | GLU | F | 232 | −0.099 | 16.838 | 4.477 | 1.00 | 50.00 | O |
| ATOM | 7756 | CB | GLU | F | 232 | 1.460 | 13.767 | 4.633 | 1.00 | 50.00 | C |
| ATOM | 7757 | CG | GLU | F | 232 | 2.859 | 14.379 | 4.644 | 1.00 | 50.00 | C |
| ATOM | 7758 | CD | GLU | F | 232 | 3.348 | 14.906 | 3.303 | 1.00 | 50.00 | C |
| ATOM | 7759 | OE1 | GLU | F | 232 | 2.639 | 15.685 | 2.631 | 1.00 | 50.00 | O |
| ATOM | 7760 | OE2 | GLU | F | 232 | 4.464 | 14.517 | 2.895 | 1.00 | 50.00 | O |
| ATOM | 7761 | N | ILE | F | 233 | −0.503 | 15.182 | 3.026 | 1.00 | 50.00 | N |
| ATOM | 7762 | CA | ILE | F | 233 | −0.869 | 15.978 | 1.857 | 1.00 | 50.00 | C |
| ATOM | 7763 | C | ILE | F | 233 | −1.898 | 17.019 | 2.274 | 1.00 | 50.00 | C |
| ATOM | 7764 | O | ILE | F | 233 | −1.593 | 18.197 | 2.452 | 1.00 | 50.00 | O |
| ATOM | 7765 | CB | ILE | F | 233 | −1.299 | 14.986 | 0.772 | 1.00 | 50.00 | C |
| ATOM | 7766 | CG1 | ILE | F | 233 | −0.148 | 14.056 | 0.384 | 1.00 | 50.00 | C |
| ATOM | 7767 | CG2 | ILE | F | 233 | −1.865 | 15.749 | −0.424 | 1.00 | 50.00 | C |
| ATOM | 7768 | CD1 | ILE | F | 233 | −0.450 | 13.065 | −0.734 | 1.00 | 50.00 | C |
| ATOM | 7769 | N | THR | F | 234 | −3.094 | 16.518 | 2.522 | 1.00 | 50.00 | N |
| ATOM | 7770 | CA | THR | F | 234 | −4.265 | 17.364 | 2.723 | 1.00 | 50.00 | C |
| ATOM | 7771 | C | THR | F | 234 | −4.576 | 17.510 | 4.210 | 1.00 | 50.00 | C |
| ATOM | 7772 | O | THR | F | 234 | −5.722 | 17.385 | 4.638 | 1.00 | 50.00 | O |
| ATOM | 7773 | CB | THR | F | 234 | −5.227 | 16.756 | 1.702 | 1.00 | 50.00 | C |
| ATOM | 7774 | OG1 | THR | F | 234 | −4.792 | 16.809 | 0.352 | 1.00 | 50.00 | O |
| ATOM | 7775 | CG2 | THR | F | 234 | −6.443 | 17.666 | 1.624 | 1.00 | 50.00 | C |
| ATOM | 7776 | N | ARG | F | 235 | −3.595 | 17.864 | 5.026 | 1.00 | 50.00 | N |
| ATOM | 7777 | CA | ARG | F | 235 | −3.740 | 18.134 | 6.457 | 1.00 | 50.00 | C |
| ATOM | 7778 | C | ARG | F | 235 | −4.551 | 19.386 | 6.780 | 1.00 | 50.00 | C |
| ATOM | 7779 | O | ARG | F | 235 | −5.503 | 19.230 | 7.542 | 1.00 | 50.00 | O |
| ATOM | 7780 | CB | APG | F | 235 | −2.414 | 17.978 | 7.204 | 1.00 | 50.00 | C |
| ATOM | 7781 | CG | ARG | F | 235 | −1.225 | 18.782 | 6.682 | 1.00 | 50.00 | C |
| ATOM | 7782 | CD | ARG | F | 235 | 0.051 | 18.498 | 7.472 | 1.00 | 50.00 | C |
| ATOM | 7783 | NE | ARG | F | 235 | 1.094 | 19.490 | 7.212 | 1.00 | 50.00 | N |
| ATOM | 7784 | CZ | ARG | F | 235 | 1.434 | 20.457 | 8.044 | 1.00 | 50.00 | C |
| ATOM | 7785 | NH1 | ARG | F | 235 | 1.109 | 20.404 | 9.319 | 1.00 | 50.00 | N |
| ATOM | 7786 | NH2 | ARG | F | 235 | 2.030 | 21.556 | 7.623 | 1.00 | 50.00 | N |
| ATOM | 7787 | N | GLU | F | 236 | −4.298 | 20.552 | 6.204 | 1.00 | 50.00 | N |
| ATOM | 7788 | CA | GLU | F | 236 | −5.146 | 21.750 | 6.191 | 1.00 | 50.00 | C |
| ATOM | 7789 | C | GLU | F | 236 | −6.656 | 21.565 | 6.239 | 1.00 | 50.00 | C |
| ATOM | 7790 | O | GLU | F | 236 | −7.312 | 21.875 | 7.233 | 1.00 | 50.00 | O |
| ATOM | 7791 | CB | GLU | F | 236 | −4.966 | 22.812 | 5.097 | 1.00 | 50.00 | C |
| ATOM | 7792 | CG | GLU | F | 236 | −4.866 | 22.463 | 3.614 | 1.00 | 50.00 | C |
| ATOM | 7793 | CD | GLU | F | 236 | −3.651 | 21.565 | 3.496 | 1.00 | 50.00 | C |
| ATOM | 7794 | OE1 | GLU | F | 236 | −3.802 | 20.344 | 3.281 | 1.00 | 50.00 | O |
| ATOM | 7795 | OE2 | GLU | F | 236 | −2.535 | 22.066 | 3.745 | 1.00 | 50.00 | O |
| ATOM | 7796 | N | PHE | F | 237 | −7.174 | 21.088 | 5.119 | 1.00 | 50.00 | N |
| ATOM | 7797 | CA | PHE | F | 237 | −8.590 | 20.821 | 4.881 | 1.00 | 50.00 | C |
| ATOM | 7798 | C | PHE | F | 237 | −9.185 | 19.901 | 5.943 | 1.00 | 50.00 | C |
| ATOM | 7799 | O | PHE | F | 237 | −10.313 | 20.094 | 6.394 | 1.00 | 50.00 | O |
| ATOM | 7800 | CB | PHE | F | 237 | −8.685 | 20.413 | 3.406 | 1.00 | 50.00 | C |
| ATOM | 7801 | CG | PHE | F | 237 | −9.114 | 19.028 | 2.967 | 1.00 | 50.00 | C |
| ATOM | 7802 | CD1 | PHE | F | 237 | −8.888 | 17.926 | 3.709 | 1.00 | 50.00 | C |
| ATOM | 7803 | CD2 | PHE | F | 237 | −9.547 | 18.830 | 1.707 | 1.00 | 50.00 | C |
| ATOM | 7804 | CE1 | PHE | F | 237 | −9.184 | 16.677 | 3.308 | 1.00 | 50.00 | C |
| ATOM | 7805 | CE2 | PHE | F | 237 | −9.714 | 17.586 | 1.213 | 1.00 | 50.00 | C |
| ATOM | 7806 | CZ | PHE | F | 237 | −9.528 | 16.518 | 2.017 | 1.00 | 50.00 | C |
| ATOM | 7807 | N | SER | F | 238 | −8.399 | 18.898 | 6.305 | 1.00 | 50.00 | N |
| ATOM | 7808 | CA | SER | F | 238 | −8.773 | 17.732 | 7.103 | 1.00 | 50.00 | C |
| ATOM | 7809 | C | SER | F | 238 | −8.823 | 18.053 | 8.589 | 1.00 | 50.00 | C |
| ATOM | 7810 | O | SER | F | 238 | −9.210 | 17.195 | 9.381 | 1.00 | 50.00 | O |
| ATOM | 7811 | CB | SER | F | 238 | −7.822 | 16.559 | 6.861 | 1.00 | 50.00 | C |
| ATOM | 7812 | OG | SER | F | 238 | −6.584 | 16.649 | 7.553 | 1.00 | 50.00 | O |
| ATOM | 7813 | N | VAL | F | 239 | −8.366 | 19.242 | 8.943 | 1.00 | 50.00 | N |
| ATOM | 7814 | CA | VAL | F | 239 | −8.334 | 19.778 | 10.297 | 1.00 | 50.00 | C |
| ATOM | 7815 | C | VAL | F | 239 | −9.204 | 21.025 | 10.425 | 1.00 | 50.00 | C |
| ATOM | 7816 | O | VAL | F | 239 | −9.804 | 21.493 | 9.458 | 1.00 | 50.00 | O |
| ATOM | 7817 | CB | VAL | F | 239 | −6.884 | 19.767 | 10.777 | 1.00 | 50.00 | C |
| ATOM | 7818 | CG1 | VAL | F | 239 | −6.180 | 18.412 | 10.715 | 1.00 | 50.00 | C |
| ATOM | 7819 | CG2 | VAL | F | 239 | −6.135 | 20.963 | 10.199 | 1.00 | 50.00 | C |
| ATOM | 7820 | N | ASN | F | 240 | −9.220 | 21.638 | 11.597 | 1.00 | 50.00 | N |
| ATOM | 7821 | CA | ASN | F | 240 | −9.735 | 22.997 | 11.777 | 1.00 | 50.00 | C |
| ATOM | 7822 | C | ASN | F | 240 | −8.853 | 24.071 | 11.142 | 1.00 | 50.00 | C |
| ATOM | 7823 | O | ASN | F | 240 | −9.433 | 25.028 | 10.631 | 1.00 | 50.00 | O |
| ATOM | 7824 | CB | ASN | F | 240 | −10.077 | 23.401 | 13.213 | 1.00 | 50.00 | C |
| ATOM | 7825 | CG | ASN | F | 240 | −10.988 | 22.706 | 14.214 | 1.00 | 50.00 | C |
| ATOM | 7826 | OD1 | ASN | F | 240 | −10.603 | 22.425 | 15.348 | 1.00 | 50.00 | O |
| ATOM | 7827 | ND2 | ASN | F | 240 | −12.233 | 22.443 | 13.867 | 1.00 | 50.00 | N |
| ATOM | 7828 | N | ALA | F | 241 | −7.529 | 24.005 | 11.156 | 1.00 | 50.00 | N |
| ATOM | 7829 | CA | ALA | F | 241 | −6.645 | 25.023 | 10.585 | 1.00 | 50.00 | C |
| ATOM | 7830 | C | ALA | F | 241 | −6.261 | 24.756 | 9.136 | 1.00 | 50.00 | C |
| ATOM | 7831 | O | ALA | F | 241 | −5.916 | 23.635 | 8.763 | 1.00 | 50.00 | O |
| ATOM | 7832 | CB | ALA | F | 241 | −5.304 | 25.122 | 11.311 | 1.00 | 50.00 | C |
| ATOM | 7833 | N | GLY | F | 242 | −6.090 | 25.834 | 8.390 | 1.00 | 50.00 | N |
| ATOM | 7834 | CA | GLY | F | 242 | −5.184 | 25.843 | 7.243 | 1.00 | 50.00 | C |
| ATOM | 7835 | C | GLY | F | 242 | −3.701 | 25.624 | 7.520 | 1.00 | 50.00 | C |
| ATOM | 7836 | O | GLY | F | 242 | −3.181 | 25.644 | 8.635 | 1.00 | 50.00 | O |
| ATOM | 7837 | N | VAL | F | 243 | −3.074 | 25.352 | 6.391 | 1.00 | 50.00 | N |
| ATOM | 7838 | CA | VAL | F | 243 | −1.835 | 24.610 | 6.147 | 1.00 | 50.00 | C |
| ATOM | 7839 | C | VAL | F | 243 | −1.714 | 24.716 | 4.628 | 1.00 | 50.00 | C |
| ATOM | 7840 | O | VAL | F | 243 | −2.562 | 25.189 | 3.870 | 1.00 | 50.00 | O |
| ATOM | 7841 | CB | VAL | F | 243 | −1.820 | 23.141 | 6.582 | 1.00 | 50.00 | C |
| ATOM | 7842 | CG1 | VAL | F | 243 | −0.680 | 22.199 | 6.202 | 1.00 | 50.00 | C |
| ATOM | 7843 | CG2 | VAL | F | 243 | −2.127 | 22.853 | 8.046 | 1.00 | 50.00 | C |
| ATOM | 7844 | N | THR | F | 244 | −0.627 | 24.181 | 4.100 | 1.00 | 50.00 | N |
| ATOM | 7845 | CA | THR | F | 244 | −0.402 | 23.974 | 2.670 | 1.00 | 50.00 | C |
| ATOM | 7846 | C | THR | F | 244 | 0.170 | 22.563 | 2.481 | 1.00 | 50.00 | C |
| ATOM | 7847 | O | THR | F | 244 | 0.520 | 21.904 | 3.459 | 1.00 | 50.00 | O |
| ATOM | 7848 | CB | THR | F | 244 | 0.164 | 25.311 | 2.172 | 1.00 | 50.00 | C |
| ATOM | 7849 | OG1 | THR | F | 244 | −0.101 | 26.471 | 2.952 | 1.00 | 50.00 | O |
| ATOM | 7850 | CG2 | THR | F | 244 | −0.341 | 25.777 | 0.809 | 1.00 | 50.00 | C |
| ATOM | 7851 | N | THR | F | 245 | 0.321 | 21.898 | 1.343 | 1.00 | 50.00 | N |
| ATOM | 7852 | CA | THR | F | 245 | −0.351 | 20.643 | 0.993 | 1.00 | 50.00 | C |
| ATOM | 7853 | C | THR | F | 245 | 0.037 | 19.693 | −0.139 | 1.00 | 50.00 | C |
| ATOM | 7854 | O | THR | F | 245 | −0.719 | 18.740 | −0.317 | 1.00 | 50.00 | O |
| ATOM | 7855 | CB | THR | F | 245 | −1.842 | 20.960 | 0.873 | 1.00 | 50.00 | C |
| ATOM | 7856 | OG1 | THR | F | 245 | −2.754 | 19.902 | 0.616 | 1.00 | 50.00 | O |
| ATOM | 7857 | CG2 | THR | F | 245 | −2.341 | 22.237 | 0.198 | 1.00 | 50.00 | C |
| ATOM | 7858 | N | PRO | F | 246 | 1.247 | 19.360 | −0.555 | 1.00 | 50.00 | N |
| ATOM | 7859 | CA | PRO | F | 246 | 1.977 | 19.935 | −1.686 | 1.00 | 50.00 | C |
| ATOM | 7860 | C | PRO | F | 246 | 2.151 | 19.219 | −3.019 | 1.00 | 50.00 | C |
| ATOM | 7861 | O | PRO | F | 246 | 2.546 | 19.849 | −3.999 | 1.00 | 50.00 | O |
| ATOM | 7862 | CB | PRO | F | 246 | 3.418 | 20.025 | −1.182 | 1.00 | 50.00 | C |
| ATOM | 7863 | CG | PRO | F | 246 | 3.501 | 18.805 | −0.271 | 1.00 | 50.00 | C |
| ATOM | 7864 | CD | PRO | F | 246 | 2.134 | 18.707 | 0.398 | 1.00 | 50.00 | C |
| ATOM | 7865 | N | VAL | F | 247 | 2.081 | 17.904 | −3.117 | 1.00 | 50.00 | N |
| ATOM | 7866 | CA | VAL | F | 247 | 0.955 | 17.080 | −2.678 | 1.00 | 50.00 | C |
| ATOM | 7867 | C | VAL | F | 247 | −0.492 | 17.521 | −2.906 | 1.00 | 50.00 | C |
| ATOM | 7868 | O | VAL | F | 247 | −1.382 | 16.684 | −3.059 | 1.00 | 50.00 | O |
| ATOM | 7869 | CB | VAL | F | 247 | 1.396 | 15.696 | −2.188 | 1.00 | 50.00 | C |
| ATOM | 7870 | CG1 | VAL | F | 247 | 1.512 | 14.490 | −3.123 | 1.00 | 50.00 | C |
| ATOM | 7871 | CG2 | VAL | F | 247 | 2.624 | 15.595 | −1.285 | 1.00 | 50.00 | C |
| ATOM | 7872 | N | SER | F | 248 | −0.765 | 18.796 | −3.131 | 1.00 | 50.00 | N |
| ATOM | 7873 | CA | SER | F | 248 | −2.031 | 19.345 | −3.617 | 1.00 | 50.00 | C |
| ATOM | 7874 | C | SER | F | 248 | −1.814 | 20.300 | −4.785 | 1.00 | 50.00 | C |
| ATOM | 7875 | O | SER | F | 248 | −2.457 | 20.035 | −5.800 | 1.00 | 50.00 | O |
| ATOM | 7876 | CB | SER | F | 248 | −2.818 | 19.977 | −2.471 | 1.00 | 50.00 | C |
| ATOM | 7877 | OG | SER | F | 248 | −3.493 | 18.978 | −1.721 | 1.00 | 50.00 | O |
| ATOM | 7878 | N | THR | F | 249 | −0.977 | 21.327 | −4.745 | 1.00 | 50.00 | N |
| ATOM | 7879 | CA | THR | F | 249 | −0.679 | 22.174 | −5.903 | 1.00 | 50.00 | C |
| ATOM | 7880 | C | THR | F | 249 | −0.281 | 21.427 | −7.173 | 1.00 | 50.00 | C |
| ATOM | 7881 | O | THR | F | 249 | −0.907 | 21.679 | −8.202 | 1.00 | 50.00 | O |
| ATOM | 7882 | CB | THR | F | 249 | 0.267 | 23.337 | −5.593 | 1.00 | 50.00 | C |
| ATOM | 7883 | OG1 | THR | F | 249 | −0.303 | 24.191 | −4.611 | 1.00 | 50.00 | O |
| ATOM | 7884 | CG2 | THR | F | 249 | 0.613 | 24.238 | −6.779 | 1.00 | 50.00 | C |
| ATOM | 7885 | N | TYR | F | 250 | 0.700 | 20.539 | −7.159 | 1.00 | 50.00 | N |
| ATOM | 7886 | CA | TYR | F | 250 | 1.052 | 19.704 | −8.310 | 1.00 | 50.00 | C |
| ATOM | 7887 | C | TYR | F | 250 | −0.031 | 18.766 | −8.840 | 1.00 | 50.00 | C |
| ATOM | 7888 | O | TYR | F | 250 | −0.104 | 18.600 | −10.056 | 1.00 | 50.00 | O |
| ATOM | 7889 | CB | TYR | F | 250 | 2.466 | 19.170 | −8.064 | 1.00 | 50.00 | C |
| ATOM | 7890 | CG | TYR | F | 250 | 3.096 | 18.053 | −8.872 | 1.00 | 50.00 | C |
| ATOM | 7891 | CD1 | TYR | F | 250 | 4.388 | 18.189 | −9.238 | 1.00 | 50.00 | C |
| ATOM | 7892 | CD2 | TYR | F | 250 | 2.456 | 16.920 | −9.232 | 1.00 | 50.00 | C |
| ATOM | 7893 | CE1 | TYR | F | 250 | 4.920 | 17.366 | −10.164 | 1.00 | 50.00 | C |
| ATOM | 7894 | CE2 | TYR | F | 250 | 3.071 | 15.962 | −9.953 | 1.00 | 50.00 | C |
| ATOM | 7895 | CZ | TYR | F | 250 | 4.251 | 16.258 | −10.532 | 1.00 | 50.00 | C |
| ATOM | 7896 | OH | TYR | F | 250 | 4.771 | 15.499 | −11.547 | 1.00 | 50.00 | O |
| ATOM | 7897 | N | MET | F | 251 | −0.840 | 18.127 | −8.009 | 1.00 | 50.00 | N |
| ATOM | 7898 | CA | MET | F | 251 | −1.881 | 17.140 | −8.315 | 1.00 | 50.00 | C |
| ATOM | 7899 | C | MET | F | 251 | −3.020 | 17.859 | −9.023 | 1.00 | 50.00 | C |
| ATOM | 7900 | O | MET | F | 251 | −3.398 | 17.412 | −10.105 | 1.00 | 50.00 | O |
| ATOM | 7901 | CB | MET | F | 251 | −2.609 | 16.389 | −7.192 | 1.00 | 50.00 | C |
| ATOM | 7902 | CG | MET | F | 251 | −2.234 | 15.032 | −6.598 | 1.00 | 50.00 | C |
| ATOM | 7903 | SD | MET | F | 251 | −1.486 | 13.865 | −7.745 | 1.00 | 50.00 | S |
| ATOM | 7904 | CE | MET | F | 251 | −2.849 | 12.697 | −7.632 | 1.00 | 50.00 | C |
| ATOM | 7905 | N | LEU | F | 252 | −3.525 | 18.926 | −8.424 | 1.00 | 50.00 | N |
| ATOM | 7906 | CA | LEU | F | 252 | −4.173 | 20.025 | −9.134 | 1.00 | 50.00 | C |
| ATOM | 7907 | C | LEU | F | 252 | −3.656 | 20.361 | −10.527 | 1.00 | 50.00 | C |
| ATOM | 7908 | O | LEU | F | 252 | −4.408 | 20.338 | −11.500 | 1.00 | 50.00 | O |
| ATOM | 7909 | CB | LEU | F | 252 | −4.263 | 21.242 | −8.215 | 1.00 | 50.00 | C |
| ATOM | 7910 | CG | LEU | F | 252 | −4.895 | 22.474 | −8.859 | 1.00 | 50.00 | C |
| ATOM | 7911 | CD1 | LEU | F | 252 | −6.276 | 22.145 | −9.425 | 1.00 | 50.00 | C |
| ATOM | 7912 | CD2 | LEU | F | 252 | −4.929 | 23.595 | −7.826 | 1.00 | 50.00 | C |
| ATOM | 7913 | N | THR | F | 253 | −2.373 | 20.661 | −10.622 | 1.00 | 50.00 | N |
| ATOM | 7914 | CA | THR | F | 253 | −1.714 | 20.879 | −11.909 | 1.00 | 50.00 | C |
| ATOM | 7915 | C | THR | F | 253 | −1.502 | 19.654 | −12.794 | 1.00 | 50.00 | C |
| ATOM | 7916 | O | THR | F | 253 | −0.960 | 19.802 | −13.889 | 1.00 | 50.00 | O |
| ATOM | 7917 | CB | THR | F | 253 | −0.475 | 21.694 | −11.538 | 1.00 | 50.00 | C |
| ATOM | 7918 | OG1 | THR | F | 253 | −0.844 | 22.875 | −10.843 | 1.00 | 50.00 | O |
| ATOM | 7919 | CG2 | THR | F | 253 | 0.327 | 22.140 | −12.756 | 1.00 | 50.00 | C |
| ATOM | 7920 | N | ASN | F | 254 | −1.937 | 18.463 | −12.411 | 1.00 | 50.00 | N |
| ATOM | 7921 | CA | ASN | F | 254 | −1.730 | 17.197 | −13.116 | 1.00 | 50.00 | C |
| ATOM | 7922 | C | ASN | F | 254 | −0.300 | 16.917 | −13.560 | 1.00 | 50.00 | C |
| ATOM | 7923 | O | ASN | F | 254 | −0.033 | 16.674 | −14.737 | 1.00 | 50.00 | O |
| ATOM | 7924 | CB | ASN | F | 254 | −2.742 | 17.033 | −14.251 | 1.00 | 50.00 | C |
| ATOM | 7925 | CG | ASN | F | 254 | −4.201 | 16.777 | −13.898 | 1.00 | 50.00 | C |
| ATOM | 7926 | OD1 | ASN | F | 254 | −5.101 | 17.452 | −14.395 | 1.00 | 50.00 | O |
| ATOM | 7927 | ND2 | ASN | F | 254 | −4.478 | 15.775 | −13.084 | 1.00 | 50.00 | N |
| ATOM | 7928 | N | SER | F | 255 | 0.643 | 17.000 | −12.637 | 1.00 | 50.00 | N |
| ATOM | 7929 | CA | SER | F | 255 | 2.070 | 16.932 | −12.948 | 1.00 | 50.00 | C |
| ATOM | 7930 | C | SER | F | 255 | 2.800 | 18.042 | −13.694 | 1.00 | 50.00 | C |
| ATOM | 7931 | O | SER | F | 255 | 4.031 | 18.043 | −13.704 | 1.00 | 50.00 | O |
| ATOM | 7932 | CB | SER | F | 255 | 2.473 | 15.525 | −13.393 | 1.00 | 50.00 | C |
| ATOM | 7933 | OG | SER | F | 255 | 2.107 | 14.518 | −12.461 | 1.00 | 50.00 | O |
| ATOM | 7934 | N | GLU | F | 256 | 2.094 | 18.994 | −14.281 | 1.00 | 50.00 | N |
| ATOM | 7935 | CA | GLU | F | 256 | 2.631 | 19.869 | −15.323 | 1.00 | 50.00 | C |
| ATOM | 7936 | C | GLU | F | 256 | 2.795 | 21.299 | −14.822 | 1.00 | 50.00 | C |
| ATOM | 7937 | O | GLU | F | 256 | 1.996 | 22.173 | −15.158 | 1.00 | 50.00 | O |
| ATOM | 7938 | CB | GLU | F | 256 | 1.699 | 19.714 | −16.527 | 1.00 | 50.00 | C |
| ATOM | 7939 | CG | GLU | F | 256 | 1.828 | 18.415 | −17.321 | 1.00 | 50.00 | C |
| ATOM | 7940 | CD | GLU | F | 256 | 3.165 | 18.272 | −18.034 | 1.00 | 50.00 | C |
| ATOM | 7941 | OE1 | GLU | F | 256 | 4.095 | 17.669 | −17.456 | 1.00 | 50.00 | O |
| ATOM | 7942 | OE2 | GLU | F | 256 | 3.304 | 18.783 | −19.166 | 1.00 | 50.00 | O |
| ATOM | 7943 | N | LEU | F | 257 | 3.796 | 21.559 | −13.998 | 1.00 | 50.00 | N |
| ATOM | 7944 | CA | LEU | F | 257 | 3.922 | 22.796 | −13.221 | 1.00 | 50.00 | C |
| ATOM | 7945 | C | LEU | F | 257 | 4.115 | 24.088 | −14.001 | 1.00 | 50.00 | C |
| ATOM | 7946 | O | LEU | F | 257 | 3.491 | 25.110 | −13.721 | 1.00 | 50.00 | O |
| ATOM | 7947 | CB | LEU | F | 257 | 5.001 | 22.685 | −12.143 | 1.00 | 50.00 | C |
| ATOM | 7948 | CG | LEU | F | 257 | 4.547 | 21.996 | −10.858 | 1.00 | 50.00 | C |
| ATOM | 7949 | CD1 | LEU | F | 257 | 3.990 | 20.600 | −11.122 | 1.00 | 50.00 | C |
| ATOM | 7950 | CD2 | LEU | F | 257 | 5.705 | 21.958 | −9.865 | 1.00 | 50.00 | C |
| ATOM | 7951 | N | LEU | F | 258 | 4.962 | 23.972 | −15.009 | 1.00 | 50.00 | N |
| ATOM | 7952 | CA | LEU | F | 258 | 5.064 | 24.836 | −16.183 | 1.00 | 50.00 | C |
| ATOM | 7953 | C | LEU | F | 258 | 3.726 | 25.314 | −16.741 | 1.00 | 50.00 | C |
| ATOM | 7954 | O | LEU | F | 258 | 3.553 | 26.527 | −16.844 | 1.00 | 50.00 | O |
| ATOM | 7955 | CB | LEU | F | 258 | 5.942 | 24.061 | −17.168 | 1.00 | 50.00 | C |
| ATOM | 7956 | CG | LEU | F | 258 | 7.390 | 23.708 | −16.816 | 1.00 | 50.00 | C |
| ATOM | 7957 | CD1 | LEU | F | 258 | 7.611 | 22.674 | −15.712 | 1.00 | 50.00 | C |
| ATOM | 7958 | CD2 | LEU | F | 258 | 8.128 | 23.187 | −18.047 | 1.00 | 50.00 | C |
| ATOM | 7959 | N | SER | F | 259 | 2.754 | 24.445 | −16.975 | 1.00 | 50.00 | N |
| ATOM | 7960 | CA | SER | F | 259 | 1.360 | 24.805 | −17.241 | 1.00 | 50.00 | C |
| ATOM | 7961 | C | SER | F | 259 | 0.663 | 25.662 | −16.188 | 1.00 | 50.00 | C |
| ATOM | 7962 | O | SER | F | 259 | 0.275 | 26.769 | −16.560 | 1.00 | 50.00 | O |
| ATOM | 7963 | CB | SER | F | 259 | 0.503 | 23.589 | −17.592 | 1.00 | 50.00 | C |
| ATOM | 7964 | OG | SER | F | 259 | 1.021 | 22.853 | −18.690 | 1.00 | 50.00 | O |
| ATOM | 7965 | N | LEU | F | 260 | 0.502 | 25.249 | −14.939 | 1.00 | 50.00 | N |
| ATOM | 7966 | CA | LEU | F | 260 | −0.147 | 26.053 | −13.900 | 1.00 | 50.00 | C |
| ATOM | 7967 | C | LEU | F | 260 | 0.456 | 27.431 | −13.665 | 1.00 | 50.00 | C |
| ATOM | 7968 | O | LEU | F | 260 | −0.306 | 28.396 | −13.635 | 1.00 | 50.00 | O |
| ATOM | 7969 | CB | LEU | F | 260 | −0.276 | 25.390 | −12.528 | 1.00 | 50.00 | C |
| ATOM | 7970 | CG | LEU | F | 260 | −1.059 | 26.123 | −11.435 | 1.00 | 50.00 | C |
| ATOM | 7971 | CD1 | LEU | F | 260 | −2.551 | 26.067 | −11.760 | 1.00 | 50.00 | C |
| ATOM | 7972 | CD2 | LEU | F | 260 | −0.712 | 25.717 | −10.003 | 1.00 | 50.00 | C |
| ATOM | 7973 | N | ILE | F | 261 | 1.759 | 27.527 | −13.459 | 1.00 | 50.00 | N |
| ATOM | 7974 | CA | ILE | F | 261 | 2.472 | 28.796 | −13.314 | 1.00 | 50.00 | C |
| ATOM | 7975 | C | ILE | F | 261 | 2.165 | 29.718 | −14.490 | 1.00 | 50.00 | C |
| ATOM | 7976 | O | ILE | F | 261 | 1.547 | 30.749 | −14.235 | 1.00 | 50.00 | O |
| ATOM | 7977 | CB | ILE | F | 261 | 3.954 | 28.575 | −13.004 | 1.00 | 50.00 | C |
| ATOM | 7978 | CG1 | ILE | F | 261 | 4.261 | 27.756 | −11.749 | 1.00 | 50.00 | C |
| ATOM | 7979 | CG2 | ILE | F | 261 | 4.678 | 29.915 | −12.879 | 1.00 | 50.00 | C |
| ATOM | 7980 | CD1 | ILE | F | 261 | 5.690 | 27.217 | −11.716 | 1.00 | 50.00 | C |
| ATOM | 7981 | N | ASN | F | 262 | 2.414 | 29.375 | −15.743 | 1.00 | 50.00 | N |
| ATOM | 7982 | CA | ASN | F | 262 | 1.933 | 30.134 | −16.900 | 1.00 | 50.00 | C |
| ATOM | 7983 | C | ASN | F | 262 | 0.488 | 30.630 | −16.910 | 1.00 | 50.00 | C |
| ATOM | 7984 | O | ASN | F | 262 | 0.260 | 31.776 | −17.296 | 1.00 | 50.00 | O |
| ATOM | 7985 | CB | ASN | F | 262 | 2.323 | 29.397 | −18.182 | 1.00 | 50.00 | C |
| ATOM | 7986 | CG | ASN | F | 262 | 3.798 | 29.206 | −18.515 | 1.00 | 50.00 | C |
| ATOM | 7987 | OD1 | ASN | F | 262 | 4.201 | 28.149 | −18.998 | 1.00 | 50.00 | O |
| ATOM | 7988 | ND2 | ASN | F | 262 | 4.657 | 30.186 | −18.303 | 1.00 | 50.00 | N |
| ATOM | 7989 | N | ASP | F | 263 | −0.468 | 29.858 | −16.416 | 1.00 | 50.00 | N |
| ATOM | 7990 | CA | ASP | F | 263 | −1.834 | 30.291 | −16.114 | 1.00 | 50.00 | C |
| ATOM | 7991 | C | ASP | F | 263 | −2.000 | 31.292 | −14.974 | 1.00 | 50.00 | C |
| ATOM | 7992 | O | ASP | F | 263 | −2.656 | 32.315 | −15.168 | 1.00 | 50.00 | O |
| ATOM | 7993 | CB | ASP | F | 263 | −2.730 | 29.074 | −15.881 | 1.00 | 50.00 | C |
| ATOM | 7994 | CG | ASP | F | 263 | −2.915 | 28.119 | −17.051 | 1.00 | 50.00 | C |
| ATOM | 7995 | OD1 | ASP | F | 263 | −3.380 | 28.560 | −18.125 | 1.00 | 50.00 | O |
| ATOM | 7996 | OD2 | ASP | F | 263 | −2.595 | 26.919 | −16.909 | 1.00 | 50.00 | O |
| ATOM | 7997 | N | MET | F | 264 | −1.437 | 31.033 | −13.804 | 1.00 | 50.00 | N |
| ATOM | 7998 | CA | MET | F | 264 | −1.433 | 31.902 | −12.627 | 1.00 | 50.00 | C |
| ATOM | 7999 | C | MET | F | 264 | −0.613 | 33.153 | −12.944 | 1.00 | 50.00 | C |
| ATOM | 8000 | O | MET | F | 264 | −1.267 | 34.155 | −13.234 | 1.00 | 50.00 | O |
| ATOM | 8001 | CB | MET | F | 264 | −1.124 | 30.948 | −11.469 | 1.00 | 50.00 | C |
| ATOM | 8002 | CG | MET | F | 264 | −1.155 | 31.426 | −10.018 | 1.00 | 50.00 | C |
| ATOM | 8003 | SD | MET | F | 264 | −0.350 | 30.316 | −8.849 | 1.00 | 50.00 | S |
| ATOM | 8004 | CE | MET | F | 264 | 1.350 | 30.067 | −9.387 | 1.00 | 50.00 | C |
| ATOM | 8005 | N | PRO | F | 265 | 0.712 | 33.179 | −13.015 | 1.00 | 50.00 | N |
| ATOM | 8006 | CA | PRO | F | 265 | 1.483 | 34.254 | −13.652 | 1.00 | 50.00 | C |
| ATOM | 8007 | C | PRO | F | 265 | 2.250 | 34.000 | −14.954 | 1.00 | 50.00 | C |
| ATOM | 8008 | O | PRO | F | 265 | 2.691 | 32.876 | −15.187 | 1.00 | 50.00 | O |
| ATOM | 8009 | CB | PRO | F | 265 | 2.318 | 34.571 | −12.411 | 1.00 | 50.00 | C |
| ATOM | 8010 | CG | PRO | F | 265 | 2.778 | 33.248 | −11.813 | 1.00 | 50.00 | C |
| ATOM | 8011 | CD | PRO | F | 265 | 1.444 | 32.528 | −11.928 | 1.00 | 50.00 | C |
| ATOM | 8012 | N | ILE | F | 266 | 2.524 | 34.967 | −15.821 | 1.00 | 50.00 | N |
| ATOM | 8013 | CA | ILE | F | 266 | 3.535 | 34.968 | −16.889 | 1.00 | 50.00 | C |
| ATOM | 8014 | C | ILE | F | 266 | 4.979 | 34.702 | −16.450 | 1.00 | 50.00 | C |
| ATOM | 8015 | O | ILE | F | 266 | 5.298 | 34.517 | −15.276 | 1.00 | 50.00 | O |
| ATOM | 8016 | CB | ILE | F | 266 | 3.368 | 36.208 | −17.783 | 1.00 | 50.00 | C |
| ATOM | 8017 | CG1 | ILE | F | 266 | 1.993 | 36.497 | −18.391 | 1.00 | 50.00 | C |
| ATOM | 8018 | CG2 | ILE | F | 266 | 4.332 | 36.383 | −18.960 | 1.00 | 50.00 | C |
| ATOM | 8019 | CD1 | ILE | F | 266 | 1.748 | 37.879 | −18.997 | 1.00 | 50.00 | C |
| ATOM | 8020 | N | THR | F | 267 | 5.960 | 34.605 | −17.335 | 1.00 | 50.00 | N |
| ATOM | 8021 | CA | THR | F | 267 | 7.396 | 34.730 | −17.068 | 1.00 | 50.00 | C |
| ATOM | 8022 | C | THR | F | 267 | 7.910 | 36.058 | −16.521 | 1.00 | 50.00 | C |
| ATOM | 8023 | O | THR | F | 267 | 8.639 | 35.997 | −15.532 | 1.00 | 50.00 | O |
| ATOM | 8024 | CB | THR | F | 267 | 8.224 | 34.357 | −18.300 | 1.00 | 50.00 | C |
| ATOM | 8025 | OG1 | THR | F | 267 | 7.906 | 33.036 | −18.714 | 1.00 | 50.00 | O |
| ATOM | 8026 | CG2 | THR | F | 267 | 9.744 | 34.425 | −18.152 | 1.00 | 50.00 | C |
| ATOM | 8027 | N | ASN | F | 268 | 7.686 | 37.205 | −17.144 | 1.00 | 50.00 | N |
| ATOM | 8028 | CA | ASN | F | 268 | 8.041 | 38.521 | −16.604 | 1.00 | 50.00 | C |
| ATOM | 8029 | C | ASN | F | 268 | 7.781 | 38.776 | −15.123 | 1.00 | 50.00 | C |
| ATOM | 8030 | O | ASN | F | 268 | 8.559 | 39.446 | −14.445 | 1.00 | 50.00 | O |
| ATOM | 8031 | CB | ASN | F | 268 | 7.494 | 39.643 | −17.491 | 1.00 | 50.00 | C |
| ATOM | 8032 | CG | ASN | F | 268 | 6.012 | 39.897 | −17.740 | 1.00 | 50.00 | C |
| ATOM | 8033 | OD1 | ASN | F | 268 | 5.643 | 40.891 | −18.364 | 1.00 | 50.00 | O |
| ATOM | 8034 | ND2 | ASN | F | 268 | 5.091 | 39.071 | −17.282 | 1.00 | 50.00 | N |
| ATOM | 8035 | N | ASP | F | 269 | 6.683 | 38.222 | −14.637 | 1.00 | 50.00 | N |
| ATOM | 8036 | CA | ASP | F | 269 | 6.289 | 38.186 | −13.230 | 1.00 | 50.00 | C |
| ATOM | 8037 | C | ASP | F | 269 | 7.043 | 37.222 | −12.320 | 1.00 | 50.00 | C |
| ATOM | 8038 | O | ASP | F | 269 | 6.903 | 37.260 | −11.100 | 1.00 | 50.00 | O |
| ATOM | 8039 | CB | ASP | F | 269 | 4.758 | 38.196 | −13.201 | 1.00 | 50.00 | C |
| ATOM | 8040 | CG | ASP | F | 269 | 3.861 | 37.451 | −14.167 | 1.00 | 50.00 | C |
| ATOM | 8041 | OD1 | ASP | F | 269 | 3.029 | 37.981 | −14.937 | 1.00 | 50.00 | O |
| ATOM | 8042 | OD2 | ASP | F | 269 | 4.060 | 36.225 | −14.078 | 1.00 | 50.00 | O |
| ATOM | 8043 | N | GLN | F | 270 | 8.008 | 36.449 | −12.785 | 1.00 | 50.00 | N |
| ATOM | 8044 | CA | GLN | F | 270 | 8.926 | 35.686 | −11.938 | 1.00 | 50.00 | C |
| ATOM | 8045 | C | GLN | F | 270 | 10.114 | 36.502 | −11.430 | 1.00 | 50.00 | C |
| ATOM | 8046 | O | GLN | F | 270 | 10.312 | 37.635 | −11.867 | 1.00 | 50.00 | O |
| ATOM | 8047 | CB | GLN | F | 270 | 9.220 | 34.358 | −12.637 | 1.00 | 50.00 | C |
| ATOM | 8048 | CG | GLN | F | 270 | 8.059 | 33.535 | −13.198 | 1.00 | 50.00 | C |
| ATOM | 8049 | CD | GLN | F | 270 | 7.094 | 32.866 | −12.232 | 1.00 | 50.00 | C |
| ATOM | 8050 | OE1 | GLN | F | 270 | 7.402 | 32.405 | −11.135 | 1.00 | 50.00 | O |
| ATOM | 8051 | NE2 | GLN | F | 270 | 5.870 | 32.699 | −12.695 | 1.00 | 50.00 | N |
| ATOM | 8052 | N | LYS | F | 271 | 10.848 | 35.981 | −10.455 | 1.00 | 50.00 | N |
| ATOM | 8053 | CA | LYS | F | 271 | 11.937 | 36.548 | −9.651 | 1.00 | 50.00 | C |
| ATOM | 8054 | C | LYS | F | 271 | 13.121 | 35.825 | −8.957 | 1.00 | 50.00 | C |
| ATOM | 8055 | O | LYS | F | 271 | 14.138 | 36.486 | −9.166 | 1.00 | 50.00 | O |
| ATOM | 8056 | CB | LYS | F | 271 | 11.465 | 37.944 | −9.208 | 1.00 | 50.00 | C |
| ATOM | 8057 | CG | LYS | F | 271 | 10.175 | 38.529 | −8.611 | 1.00 | 50.00 | C |
| ATOM | 8058 | CD | LYS | F | 271 | 8.923 | 39.170 | −9.225 | 1.00 | 50.00 | C |
| ATOM | 8059 | CE | LYS | F | 271 | 8.296 | 40.567 | −9.299 | 1.00 | 50.00 | C |
| ATOM | 8060 | NZ | LYS | F | 271 | 8.485 | 41.617 | −10.310 | 1.00 | 50.00 | N |
| ATOM | 8061 | N | LYS | F | 272 | 13.675 | 34.786 | −8.317 | 1.00 | 50.00 | N |
| ATOM | 8062 | CA | LYS | F | 272 | 13.564 | 33.747 | −7.269 | 1.00 | 50.00 | C |
| ATOM | 8063 | C | LYS | F | 272 | 13.083 | 32.283 | −7.254 | 1.00 | 50.00 | C |
| ATOM | 8064 | O | LYS | F | 272 | 14.000 | 31.483 | −7.070 | 1.00 | 50.00 | O |
| ATOM | 8065 | CB | LYS | F | 272 | 13.951 | 34.055 | −5.813 | 1.00 | 50.00 | C |
| ATOM | 8066 | CG | LYS | F | 272 | 13.943 | 33.082 | −4.623 | 1.00 | 50.00 | C |
| ATOM | 8067 | CD | LYS | F | 272 | 15.132 | 32.889 | −3.680 | 1.00 | 50.00 | C |
| ATOM | 8068 | CE | LYS | F | 272 | 15.145 | 31.609 | −2.842 | 1.00 | 50.00 | C |
| ATOM | 8069 | NZ | LYS | F | 272 | 16.125 | 31.650 | −1.746 | 1.00 | 50.00 | N |
| ATOM | 8070 | N | LEU | F | 273 | 12.012 | 31.490 | −7.249 | 1.00 | 50.00 | N |
| ATOM | 8071 | CA | LEU | F | 273 | 10.572 | 31.633 | −6.996 | 1.00 | 50.00 | C |
| ATOM | 8072 | C | LEU | F | 273 | 9.583 | 30.881 | −7.889 | 1.00 | 50.00 | C |
| ATOM | 8073 | O | LEU | F | 273 | 8.991 | 31.400 | −8.834 | 1.00 | 50.00 | O |
| ATOM | 8074 | CB | LEU | F | 273 | 9.938 | 33.003 | −6.759 | 1.00 | 50.00 | C |
| ATOM | 8075 | CG | LEU | F | 273 | 10.454 | 34.086 | −7.702 | 1.00 | 50.00 | C |
| ATOM | 8076 | CD1 | LEU | F | 273 | 10.806 | 33.576 | −9.106 | 1.00 | 50.00 | C |
| ATOM | 8077 | CD2 | LEU | F | 273 | 10.648 | 35.426 | −6.990 | 1.00 | 50.00 | C |
| ATOM | 8078 | N | MET | F | 274 | 9.174 | 29.659 | −7.592 | 1.00 | 50.00 | N |
| ATOM | 8079 | CA | MET | F | 274 | 7.840 | 29.406 | −7.037 | 1.00 | 50.00 | C |
| ATOM | 8080 | C | MET | F | 274 | 7.670 | 29.691 | −5.547 | 1.00 | 50.00 | C |
| ATOM | 8081 | O | MET | F | 274 | 6.543 | 29.745 | −5.057 | 1.00 | 50.00 | O |
| ATOM | 8082 | CB | MET | F | 274 | 7.464 | 27.952 | −7.331 | 1.00 | 50.00 | C |
| ATOM | 8083 | CG | MET | F | 274 | 6.487 | 27.609 | −8.453 | 1.00 | 50.00 | C |
| ATOM | 8084 | SD | MET | F | 274 | 4.789 | 27.553 | −7.852 | 1.00 | 50.00 | S |
| ATOM | 8085 | CE | MET | F | 274 | 4.372 | 25.809 | −8.027 | 1.00 | 50.00 | C |
| ATOM | 8086 | N | SER | F | 275 | 8.762 | 29.843 | −4.816 | 1.00 | 50.00 | N |
| ATOM | 8087 | CA | SER | F | 275 | 8.890 | 29.873 | −3.359 | 1.00 | 50.00 | C |
| ATOM | 8088 | C | SER | F | 275 | 8.213 | 31.042 | −2.661 | 1.00 | 50.00 | C |
| ATOM | 8089 | O | SER | F | 275 | 7.517 | 30.854 | −1.665 | 1.00 | 50.00 | O |
| ATOM | 8090 | CB | SER | F | 275 | 10.372 | 29.886 | −2.980 | 1.00 | 50.00 | C |
| ATOM | 8091 | OG | SER | F | 275 | 11.079 | 31.029 | −3.441 | 1.00 | 50.00 | O |
| ATOM | 8092 | N | ASN | F | 276 | 8.415 | 32.223 | −3.218 | 1.00 | 50.00 | N |
| ATOM | 8093 | CA | ASN | F | 276 | 7.679 | 33.437 | −2.873 | 1.00 | 50.00 | C |
| ATOM | 8094 | C | ASN | F | 276 | 6.178 | 33.229 | −3.008 | 1.00 | 50.00 | C |
| ATOM | 8095 | O | ASN | F | 276 | 5.544 | 33.206 | −1.955 | 1.00 | 50.00 | O |
| ATOM | 8096 | CB | ASN | F | 276 | 8.253 | 34.592 | −3.691 | 1.00 | 50.00 | C |
| ATOM | 8097 | CG | ASN | F | 276 | 9.712 | 34.869 | −3.360 | 1.00 | 50.00 | C |
| ATOM | 8098 | OD1 | ASN | F | 276 | 10.591 | 34.747 | −4.208 | 1.00 | 50.00 | O |
| ATOM | 8099 | ND2 | ASN | F | 276 | 10.135 | 35.078 | −2.129 | 1.00 | 50.00 | N |
| ATOM | 8100 | N | ASN | F | 277 | 5.661 | 32.885 | −4.176 | 1.00 | 50.00 | N |
| ATOM | 8101 | CA | ASN | F | 277 | 4.294 | 32.396 | −4.357 | 1.00 | 50.00 | C |
| ATOM | 8102 | C | ASN | F | 277 | 3.742 | 31.457 | −3.291 | 1.00 | 50.00 | C |
| ATOM | 8103 | O | ASN | F | 277 | 2.829 | 31.891 | −2.589 | 1.00 | 50.00 | O |
| ATOM | 8104 | CB | ASN | F | 277 | 4.088 | 31.974 | −5.812 | 1.00 | 50.00 | C |
| ATOM | 8105 | CG | ASN | F | 277 | 4.332 | 33.009 | −6.903 | 1.00 | 50.00 | C |
| ATOM | 8106 | OD1 | ASN | F | 277 | 4.707 | 32.643 | −8.016 | 1.00 | 50.00 | O |
| ATOM | 8107 | ND2 | ASN | F | 277 | 4.068 | 34.283 | −6.670 | 1.00 | 50.00 | N |
| ATOM | 8108 | N | VAL | F | 278 | 4.360 | 30.314 | −3.041 | 1.00 | 50.00 | N |
| ATOM | 8109 | CA | VAL | F | 278 | 4.119 | 29.441 | −1.889 | 1.00 | 50.00 | C |
| ATOM | 8110 | C | VAL | F | 278 | 4.041 | 30.140 | −0.533 | 1.00 | 50.00 | C |
| ATOM | 8111 | O | VAL | F | 278 | 3.030 | 30.038 | 0.161 | 1.00 | 50.00 | O |
| ATOM | 8112 | CB | VAL | F | 278 | 5.094 | 28.260 | −1.909 | 1.00 | 50.00 | C |
| ATOM | 8113 | CG1 | VAL | F | 278 | 5.041 | 27.348 | −0.682 | 1.00 | 50.00 | C |
| ATOM | 8114 | CG2 | VAL | F | 278 | 4.956 | 27.363 | −3.139 | 1.00 | 50.00 | C |
| ATOM | 8115 | N | GLN | F | 279 | 5.097 | 30.825 | −0.128 | 1.00 | 50.00 | N |
| ATOM | 8116 | CA | GLN | F | 279 | 5.160 | 31.582 | 1.122 | 1.00 | 50.00 | C |
| ATOM | 8117 | C | GLN | F | 279 | 4.237 | 32.783 | 1.287 | 1.00 | 50.00 | C |
| ATOM | 8118 | O | GLN | F | 279 | 3.970 | 33.144 | 2.433 | 1.00 | 50.00 | O |
| ATOM | 8119 | CB | GLN | F | 279 | 6.620 | 31.972 | 1.355 | 1.00 | 50.00 | C |
| ATOM | 8120 | CG | GLN | F | 279 | 7.612 | 30.862 | 1.699 | 1.00 | 50.00 | C |
| ATOM | 8121 | CD | GLN | F | 279 | 7.264 | 30.111 | 2.976 | 1.00 | 50.00 | C |
| ATOM | 8122 | OE1 | GLN | F | 279 | 6.729 | 30.637 | 3.951 | 1.00 | 50.00 | O |
| ATOM | 8123 | NE2 | GLN | F | 279 | 7.554 | 28.824 | 2.971 | 1.00 | 50.00 | N |
| ATOM | 8124 | N | ILE | F | 280 | 3.736 | 33.395 | 0.225 | 1.00 | 50.00 | N |
| ATOM | 8125 | CA | ILE | F | 280 | 2.758 | 34.486 | 0.254 | 1.00 | 50.00 | C |
| ATOM | 8126 | C | ILE | F | 280 | 1.485 | 34.195 | 1.036 | 1.00 | 50.00 | C |
| ATOM | 8127 | O | ILE | F | 280 | 0.941 | 35.156 | 1.578 | 1.00 | 50.00 | O |
| ATOM | 8128 | CB | ILE | F | 280 | 2.452 | 35.028 | −1.145 | 1.00 | 50.00 | C |
| ATOM | 8129 | CG1 | ILE | F | 280 | 3.705 | 35.743 | −1.645 | 1.00 | 50.00 | C |
| ATOM | 8130 | CG2 | ILE | F | 280 | 1.317 | 36.041 | −1.290 | 1.00 | 50.00 | C |
| ATOM | 8131 | CD1 | ILE | F | 280 | 3.934 | 35.976 | −3.131 | 1.00 | 50.00 | C |
| ATOM | 8132 | N | VAL | F | 281 | 1.013 | 32.959 | 1.064 | 1.00 | 50.00 | N |
| ATOM | 8133 | CA | VAL | F | 281 | −0.276 | 32.585 | 1.651 | 1.00 | 50.00 | C |
| ATOM | 8134 | C | VAL | F | 281 | −1.468 | 32.904 | 0.749 | 1.00 | 50.00 | C |
| ATOM | 8135 | O | VAL | F | 281 | −2.619 | 32.833 | 1.180 | 1.00 | 50.00 | O |
| ATOM | 8136 | CB | VAL | F | 281 | −0.278 | 32.932 | 3.145 | 1.00 | 50.00 | C |
| ATOM | 8137 | CG1 | VAL | F | 281 | −1.591 | 32.863 | 3.920 | 1.00 | 50.00 | C |
| ATOM | 8138 | CG2 | VAL | F | 281 | 0.814 | 32.221 | 3.945 | 1.00 | 50.00 | C |
| ATOM | 8139 | N | ARG | F | 282 | −1.268 | 33.169 | −0.532 | 1.00 | 50.00 | N |
| ATOM | 8140 | CA | ARG | F | 282 | −2.288 | 33.410 | −1.553 | 1.00 | 50.00 | C |
| ATOM | 8141 | C | ARG | F | 282 | −3.062 | 32.192 | −2.050 | 1.00 | 50.00 | C |
| ATOM | 8142 | O | ARG | F | 282 | −4.281 | 32.267 | −2.198 | 1.00 | 50.00 | O |
| ATOM | 8143 | CB | ARG | F | 282 | −1.583 | 34.172 | −2.676 | 1.00 | 50.00 | C |
| ATOM | 8144 | CG | ARG | F | 282 | −0.498 | 33.397 | −3.427 | 1.00 | 50.00 | C |
| ATOM | 8145 | CD | ARG | F | 282 | 0.201 | 34.160 | −4.547 | 1.00 | 50.00 | C |
| ATOM | 8146 | NE | ARG | F | 282 | 0.737 | 33.296 | −5.599 | 1.00 | 50.00 | N |
| ATOM | 8147 | CZ | ARG | F | 282 | 0.885 | 33.607 | −6.877 | 1.00 | 50.00 | C |
| ATOM | 8148 | NH1 | ARG | F | 282 | 0.605 | 34.768 | −7.441 | 1.00 | 50.00 | N |
| ATOM | 8149 | NH2 | ARG | F | 282 | 1.350 | 32.676 | −7.686 | 1.00 | 50.00 | N |
| ATOM | 8150 | N | GLN | F | 283 | −2.375 | 31.094 | −2.321 | 1.00 | 50.00 | N |
| ATOM | 8151 | CA | GLN | F | 283 | −2.913 | 29.827 | −2.815 | 1.00 | 50.00 | C |
| ATOM | 8152 | C | GLN | F | 283 | −3.454 | 29.027 | −1.636 | 1.00 | 50.00 | C |
| ATOM | 8153 | O | GLN | F | 283 | −2.719 | 28.303 | −0.964 | 1.00 | 50.00 | O |
| ATOM | 8154 | CB | GLN | F | 283 | −1.878 | 29.062 | −3.643 | 1.00 | 50.00 | C |
| ATOM | 8155 | CG | GLN | F | 283 | −0.522 | 28.676 | −3.051 | 1.00 | 50.00 | C |
| ATOM | 8156 | CD | GLN | F | 283 | 0.423 | 29.855 | −2.874 | 1.00 | 50.00 | C |
| ATOM | 8157 | OE1 | GLN | F | 283 | 0.826 | 30.489 | −3.849 | 1.00 | 50.00 | O |
| ATOM | 8158 | NE2 | GLN | F | 283 | 0.741 | 30.200 | −1.640 | 1.00 | 50.00 | N |
| ATOM | 8159 | N | GLN | F | 284 | −4.733 | 29.202 | −1.348 | 1.00 | 50.00 | N |
| ATOM | 8160 | CA | GLN | F | 284 | −5.373 | 28.611 | −0.173 | 1.00 | 50.00 | C |
| ATOM | 8161 | C | GLN | F | 284 | −6.568 | 27.755 | −0.570 | 1.00 | 50.00 | C |
| ATOM | 8162 | O | GLN | F | 284 | −7.225 | 27.993 | −1.582 | 1.00 | 50.00 | O |
| ATOM | 8163 | CB | GLN | F | 284 | −5.757 | 29.677 | 0.854 | 1.00 | 50.00 | C |
| ATOM | 8164 | CG | GLN | F | 284 | −4.581 | 30.239 | 1.652 | 1.00 | 50.00 | C |
| ATOM | 8165 | CD | GLN | F | 284 | −3.840 | 29.251 | 2.543 | 1.00 | 50.00 | C |
| ATOM | 8166 | OE1 | GLN | F | 284 | −4.288 | 28.146 | 2.846 | 1.00 | 50.00 | O |
| ATOM | 8167 | NE2 | GLN | F | 284 | −2.656 | 29.620 | 2.992 | 1.00 | 50.00 | N |
| ATOM | 8168 | N | SER | F | 285 | −6.826 | 26.754 | 0.256 | 1.00 | 50.00 | N |
| ATOM | 8169 | CA | SER | F | 285 | −7.833 | 25.726 | −0.003 | 1.00 | 50.00 | C |
| ATOM | 8170 | C | SER | F | 285 | −8.624 | 25.391 | 1.254 | 1.00 | 50.00 | C |
| ATOM | 8171 | O | SER | F | 285 | −8.122 | 25.496 | 2.373 | 1.00 | 50.00 | O |
| ATOM | 8172 | CB | SER | F | 285 | −7.192 | 24.452 | −0.551 | 1.00 | 50.00 | C |
| ATOM | 8173 | OG | SER | F | 285 | −6.353 | 24.694 | −1.671 | 1.00 | 50.00 | O |
| ATOM | 8174 | N | TYR | F | 286 | −9.865 | 24.975 | 1.062 | 1.00 | 50.00 | N |
| ATOM | 8175 | CA | TYR | F | 286 | −10.792 | 24.688 | 2.157 | 1.00 | 50.00 | C |
| ATOM | 8176 | C | TYR | F | 286 | −11.704 | 23.535 | 1.762 | 1.00 | 50.00 | C |
| ATOM | 8177 | O | TYR | F | 286 | −12.301 | 23.555 | 0.687 | 1.00 | 50.00 | O |
| ATOM | 8178 | CB | TYR | F | 286 | −11.622 | 25.914 | 2.545 | 1.00 | 50.00 | C |
| ATOM | 8179 | CG | TYR | F | 286 | −10.834 | 26.969 | 3.291 | 1.00 | 50.00 | C |
| ATOM | 8180 | CD1 | TYR | F | 286 | −10.391 | 26.677 | 4.531 | 1.00 | 50.00 | C |
| ATOM | 8181 | CD2 | TYR | F | 286 | −10.393 | 28.084 | 2.671 | 1.00 | 50.00 | C |
| ATOM | 8182 | CE1 | TYR | F | 286 | −9.460 | 27.455 | 5.117 | 1.00 | 50.00 | C |
| ATOM | 8183 | CE2 | TYR | F | 286 | −9.491 | 28.886 | 3.274 | 1.00 | 50.00 | C |
| ATOM | 8184 | CZ | TYR | F | 286 | −9.022 | 28.570 | 4.498 | 1.00 | 50.00 | C |
| ATOM | 8185 | OH | TYR | F | 286 | −8.078 | 29.346 | 5.118 | 1.00 | 50.00 | O |
| ATOM | 8186 | N | SER | F | 287 | −11.934 | 22.614 | 2.684 | 1.00 | 50.00 | N |
| ATOM | 8187 | CA | SER | F | 287 | −13.016 | 21.634 | 2.584 | 1.00 | 50.00 | C |
| ATOM | 8188 | C | SER | F | 287 | −14.369 | 22.298 | 2.347 | 1.00 | 50.00 | C |
| ATOM | 8189 | O | SER | F | 287 | −14.645 | 23.376 | 2.874 | 1.00 | 50.00 | O |
| ATOM | 8190 | CB | SER | F | 287 | −13.031 | 20.747 | 3.829 | 1.00 | 50.00 | C |
| ATOM | 8191 | OG | SER | F | 287 | −11.851 | 19.964 | 3.936 | 1.00 | 50.00 | O |
| ATOM | 8192 | N | ILE | F | 288 | −15.159 | 21.701 | 1.472 | 1.00 | 50.00 | N |
| ATOM | 8193 | CA | ILE | F | 288 | −16.538 | 22.079 | 1.154 | 1.00 | 50.00 | C |
| ATOM | 8194 | C | ILE | F | 288 | −17.539 | 20.936 | 1.298 | 1.00 | 50.00 | C |
| ATOM | 8195 | O | ILE | F | 288 | −18.717 | 21.212 | 1.523 | 1.00 | 50.00 | O |
| ATOM | 8196 | CB | ILE | F | 288 | −16.693 | 22.791 | −0.193 | 1.00 | 50.00 | C |
| ATOM | 8197 | CG1 | ILE | F | 288 | −16.154 | 21.984 | −1.375 | 1.00 | 50.00 | C |
| ATOM | 8198 | CG2 | ILE | F | 288 | −16.159 | 24.223 | −0.150 | 1.00 | 50.00 | C |
| ATOM | 8199 | CD1 | ILE | F | 288 | −16.786 | 22.249 | −2.739 | 1.00 | 50.00 | C |
| ATOM | 8200 | N | MET | F | 289 | −17.145 | 19.688 | 1.101 | 1.00 | 50.00 | N |
| ATOM | 8201 | CA | MET | F | 289 | −18.035 | 18.535 | 0.960 | 1.00 | 50.00 | C |
| ATOM | 8202 | C | MET | F | 289 | −17.345 | 17.337 | 1.599 | 1.00 | 50.00 | C |
| ATOM | 8203 | O | MET | F | 289 | −16.158 | 17.104 | 1.372 | 1.00 | 50.00 | O |
| ATOM | 8204 | CB | MET | F | 289 | −18.312 | 18.162 | −0.496 | 1.00 | 50.00 | C |
| ATOM | 8205 | CG | MET | F | 289 | −19.073 | 19.186 | −1.335 | 1.00 | 50.00 | C |
| ATOM | 8206 | SD | MET | F | 289 | −20.750 | 19.374 | −0.708 | 1.00 | 50.00 | S |
| ATOM | 8207 | CE | MET | F | 289 | −21.500 | 17.826 | −1.241 | 1.00 | 50.00 | C |
| ATOM | 8208 | N | SER | F | 290 | −18.113 | 16.547 | 2.330 | 1.00 | 50.00 | N |
| ATOM | 8209 | CA | SER | F | 290 | −17.657 | 15.285 | 2.911 | 1.00 | 50.00 | C |
| ATOM | 8210 | C | SER | F | 290 | −18.809 | 14.301 | 2.748 | 1.00 | 50.00 | C |
| ATOM | 8211 | O | SER | F | 290 | −19.743 | 14.311 | 3.550 | 1.00 | 50.00 | O |
| ATOM | 8212 | CB | SER | F | 290 | −17.304 | 15.465 | 4.387 | 1.00 | 50.00 | C |
| ATOM | 8213 | OG | SER | F | 290 | −16.170 | 16.288 | 4.612 | 1.00 | 50.00 | O |
| ATOM | 8214 | N | ILE | F | 291 | −18.751 | 13.429 | 1.753 | 1.00 | 50.00 | N |
| ATOM | 8215 | CA | ILE | F | 291 | −19.691 | 12.316 | 1.607 | 1.00 | 50.00 | C |
| ATOM | 8216 | C | ILE | F | 291 | −19.213 | 11.246 | 2.582 | 1.00 | 50.00 | C |
| ATOM | 8217 | O | ILE | F | 291 | −18.228 | 10.556 | 2.322 | 1.00 | 50.00 | O |
| ATOM | 8218 | CB | ILE | F | 291 | −19.859 | 11.816 | 0.168 | 1.00 | 50.00 | C |
| ATOM | 8219 | CG1 | ILE | F | 291 | −20.241 | 12.879 | −0.864 | 1.00 | 50.00 | C |
| ATOM | 8220 | CG2 | ILE | F | 291 | −20.851 | 10.656 | 0.071 | 1.00 | 50.00 | C |
| ATOM | 8221 | CD1 | ILE | F | 291 | −20.003 | 12.523 | −2.331 | 1.00 | 50.00 | C |
| ATOM | 8222 | N | ILE | F | 292 | −19.916 | 11.099 | 3.694 | 1.00 | 50.00 | N |
| ATOM | 8223 | CA | ILE | F | 292 | −19.690 | 10.057 | 4.699 | 1.00 | 50.00 | C |
| ATOM | 8224 | C | ILE | F | 292 | −19.741 | 8.636 | 4.141 | 1.00 | 50.00 | C |
| ATOM | 8225 | O | ILE | F | 292 | −18.876 | 7.825 | 4.471 | 1.00 | 50.00 | O |
| ATOM | 8226 | CB | ILE | F | 292 | −20.576 | 10.318 | 5.923 | 1.00 | 50.00 | C |
| ATOM | 8227 | CG1 | ILE | F | 292 | −20.171 | 11.572 | 6.703 | 1.00 | 50.00 | C |
| ATOM | 8228 | CG2 | ILE | F | 292 | −20.683 | 9.118 | 6.865 | 1.00 | 50.00 | C |
| ATOM | 8229 | CD1 | ILE | F | 292 | −21.105 | 12.045 | 7.815 | 1.00 | 50.00 | C |
| ATOM | 8230 | N | LYS | F | 293 | −20.753 | 8.308 | 3.355 | 1.00 | 50.00 | N |
| ATOM | 8231 | CA | LYS | F | 293 | −21.026 | 6.959 | 2.857 | 1.00 | 50.00 | C |
| ATOM | 8232 | C | LYS | F | 293 | −20.053 | 6.457 | 1.797 | 1.00 | 50.00 | C |
| ATOM | 8233 | O | LYS | F | 293 | −19.411 | 5.428 | 2.005 | 1.00 | 50.00 | O |
| ATOM | 8234 | CB | LYS | F | 293 | −22.469 | 6.922 | 2.353 | 1.00 | 50.00 | C |
| ATOM | 8235 | CG | LYS | F | 293 | −23.536 | 7.089 | 3.434 | 1.00 | 50.00 | C |
| ATOM | 8236 | CD | LYS | F | 293 | −24.946 | 7.002 | 2.854 | 1.00 | 50.00 | C |
| ATOM | 8237 | CE | LYS | F | 293 | −26.007 | 7.222 | 3.929 | 1.00 | 50.00 | C |
| ATOM | 8238 | NZ | LYS | F | 293 | −27.352 | 7.057 | 3.359 | 1.00 | 50.00 | N |
| ATOM | 8239 | N | GLU | F | 294 | −19.920 | 7.172 | 0.691 | 1.00 | 50.00 | N |
| ATOM | 8240 | CA | GLU | F | 294 | −18.888 | 6.941 | −0.320 | 1.00 | 50.00 | C |
| ATOM | 8241 | C | GLU | F | 294 | −17.440 | 7.176 | 0.095 | 1.00 | 50.00 | C |
| ATOM | 8242 | O | GLU | F | 294 | −16.550 | 6.668 | −0.586 | 1.00 | 50.00 | O |
| ATOM | 8243 | CB | GLU | F | 294 | −19.191 | 7.781 | −1.562 | 1.00 | 50.00 | C |
| ATOM | 8244 | CG | GLU | F | 294 | −20.492 | 7.475 | −2.303 | 1.00 | 50.00 | C |
| ATOM | 8245 | CD | GLU | F | 294 | −20.476 | 6.121 | −2.996 | 1.00 | 50.00 | C |
| ATOM | 8246 | OE1 | GLU | F | 294 | −20.814 | 5.107 | −2.348 | 1.00 | 50.00 | O |
| ATOM | 8247 | OE2 | GLU | F | 294 | −20.102 | 6.057 | −4.187 | 1.00 | 50.00 | O |
| ATOM | 8248 | N | GLU | F | 295 | −17.179 | 7.933 | 1.149 | 1.00 | 50.00 | N |
| ATOM | 8249 | CA | GLU | F | 295 | −15.839 | 8.286 | 1.616 | 1.00 | 50.00 | C |
| ATOM | 8250 | C | GLU | F | 295 | −15.142 | 9.183 | 0.598 | 1.00 | 50.00 | C |
| ATOM | 8251 | O | GLU | F | 295 | −14.218 | 8.756 | −0.093 | 1.00 | 50.00 | O |
| ATOM | 8252 | CB | GLU | F | 295 | −15.086 | 7.063 | 2.145 | 1.00 | 50.00 | C |
| ATOM | 8253 | CG | GLU | F | 295 | −15.706 | 6.277 | 3.300 | 1.00 | 50.00 | C |
| ATOM | 8254 | CD | GLU | F | 295 | −15.504 | 6.979 | 4.633 | 1.00 | 50.00 | C |
| ATOM | 8255 | OE1 | GLU | F | 295 | −16.096 | 8.066 | 4.806 | 1.00 | 50.00 | O |
| ATOM | 8256 | OE2 | GLU | F | 295 | −14.737 | 6.488 | 5.488 | 1.00 | 50.00 | O |
| ATOM | 8257 | N | VAL | F | 296 | −15.628 | 10.408 | 0.471 | 1.00 | 50.00 | N |
| ATOM | 8258 | CA | VAL | F | 296 | −15.212 | 11.383 | −0.538 | 1.00 | 50.00 | C |
| ATOM | 8259 | C | VAL | F | 296 | −15.042 | 12.710 | 0.194 | 1.00 | 50.00 | C |
| ATOM | 8260 | O | VAL | F | 296 | −15.835 | 13.075 | 1.062 | 1.00 | 50.00 | O |
| ATOM | 8261 | CB | VAL | F | 296 | −16.204 | 11.493 | −1.699 | 1.00 | 50.00 | C |
| ATOM | 8262 | CG1 | VAL | F | 296 | −15.956 | 12.627 | −2.695 | 1.00 | 50.00 | C |
| ATOM | 8263 | CG2 | VAL | F | 296 | −16.358 | 10.193 | −2.487 | 1.00 | 50.00 | C |
| ATOM | 8264 | N | LEU | F | 297 | −14.058 | 13.482 | −0.235 | 1.00 | 50.00 | N |
| ATOM | 8265 | CA | LEU | F | 297 | −13.897 | 14.877 | 0.173 | 1.00 | 50.00 | C |
| ATOM | 8266 | C | LEU | F | 297 | −13.669 | 15.769 | −1.041 | 1.00 | 50.00 | C |
| ATOM | 8267 | O | LEU | F | 297 | −12.720 | 15.542 | −1.791 | 1.00 | 50.00 | O |
| ATOM | 8268 | CB | LEU | F | 297 | −12.737 | 14.950 | 1.166 | 1.00 | 50.00 | C |
| ATOM | 8269 | CG | LEU | F | 297 | −12.676 | 16.137 | 2.128 | 1.00 | 50.00 | C |
| ATOM | 8270 | CD1 | LEU | F | 297 | −12.720 | 17.511 | 1.464 | 1.00 | 50.00 | C |
| ATOM | 8271 | CD2 | LEU | F | 297 | −13.652 | 16.145 | 3.299 | 1.00 | 50.00 | C |
| ATOM | 8272 | N | ALA | F | 298 | −14.433 | 16.840 | −1.184 | 1.00 | 50.00 | N |
| ATOM | 8273 | CA | ALA | F | 298 | −14.090 | 17.959 | −2.062 | 1.00 | 50.00 | C |
| ATOM | 8274 | C | ALA | F | 298 | −13.428 | 19.117 | −1.323 | 1.00 | 50.00 | C |
| ATOM | 8275 | O | ALA | F | 298 | −14.054 | 19.736 | −0.464 | 1.00 | 50.00 | O |
| ATOM | 8276 | CB | ALA | F | 298 | −15.359 | 18.459 | −2.750 | 1.00 | 50.00 | C |
| ATOM | 8277 | N | TYR | F | 299 | −12.195 | 19.456 | −1.665 | 1.00 | 50.00 | N |
| ATOM | 8278 | CA | TYR | F | 299 | −11.590 | 20.755 | −1.361 | 1.00 | 50.00 | C |
| ATOM | 8279 | C | TYR | F | 299 | −12.110 | 21.857 | −2.281 | 1.00 | 50.00 | C |
| ATOM | 8280 | O | TYR | F | 299 | −12.718 | 21.571 | −3.312 | 1.00 | 50.00 | O |
| ATOM | 8281 | CB | TYR | F | 299 | −10.070 | 20.608 | −1.231 | 1.00 | 50.00 | C |
| ATOM | 8282 | CG | TYR | F | 299 | −9.003 | 21.110 | −2.181 | 1.00 | 50.00 | C |
| ATOM | 8283 | CD1 | TYR | F | 299 | −9.305 | 21.442 | −3.452 | 1.00 | 50.00 | C |
| ATOM | 8284 | CD2 | TYR | F | 299 | −7.722 | 21.223 | −1.769 | 1.00 | 50.00 | C |
| ATOM | 8285 | CE1 | TYR | F | 299 | −8.367 | 21.925 | −4.289 | 1.00 | 50.00 | C |
| ATOM | 8286 | CE2 | TYR | F | 299 | −6.767 | 21.681 | −2.603 | 1.00 | 50.00 | C |
| ATOM | 8287 | CZ | TYR | F | 299 | −7.088 | 22.005 | −3.871 | 1.00 | 50.00 | C |
| ATOM | 8288 | OH | TYR | F | 299 | −6.104 | 22.378 | −4.748 | 1.00 | 50.00 | O |
| ATOM | 8289 | N | VAL | F | 300 | −11.774 | 23.110 | −2.019 | 1.00 | 50.00 | N |
| ATOM | 8290 | CA | VAL | F | 300 | −11.812 | 24.195 | −3.001 | 1.00 | 50.00 | C |
| ATOM | 8291 | C | VAL | F | 300 | −10.558 | 25.032 | −2.774 | 1.00 | 50.00 | C |
| ATOM | 8292 | O | VAL | F | 300 | −10.536 | 25.865 | −1.868 | 1.00 | 50.00 | O |
| ATOM | 8293 | CB | VAL | F | 300 | −13.081 | 25.048 | −2.928 | 1.00 | 50.00 | C |
| ATOM | 8294 | CG1 | VAL | F | 300 | −13.081 | 26.365 | −3.706 | 1.00 | 50.00 | C |
| ATOM | 8295 | CG2 | VAL | F | 300 | −14.329 | 24.326 | −3.429 | 1.00 | 50.00 | C |
| ATOM | 8296 | N | VAL | F | 301 | −9.592 | 24.932 | −3.672 | 1.00 | 50.00 | N |
| ATOM | 8297 | CA | VAL | F | 301 | −8.532 | 25.925 | −3.855 | 1.00 | 50.00 | C |
| ATOM | 8298 | C | VAL | F | 301 | −9.037 | 27.218 | −4.485 | 1.00 | 50.00 | C |
| ATOM | 8299 | O | VAL | F | 301 | −10.077 | 27.296 | −5.139 | 1.00 | 50.00 | O |
| ATOM | 8300 | CB | VAL | F | 301 | −7.344 | 25.332 | −4.613 | 1.00 | 50.00 | C |
| ATOM | 8301 | CG1 | VAL | F | 301 | −7.734 | 24.968 | −6.043 | 1.00 | 50.00 | C |
| ATOM | 8302 | CG2 | VAL | F | 301 | −5.983 | 26.027 | −4.580 | 1.00 | 50.00 | C |
| ATOM | 8303 | N | GLN | F | 302 | −8.211 | 28.230 | −4.287 | 1.00 | 50.00 | N |
| ATOM | 8304 | CA | GLN | F | 302 | −8.441 | 29.596 | −4.750 | 1.00 | 50.00 | C |
| ATOM | 8305 | C | GLN | F | 302 | −7.059 | 30.112 | −5.127 | 1.00 | 50.00 | C |
| ATOM | 8306 | O | GLN | F | 302 | −6.303 | 30.532 | −4.251 | 1.00 | 50.00 | O |
| ATOM | 8307 | CB | GLN | F | 302 | −9.090 | 30.391 | −3.616 | 1.00 | 50.00 | C |
| ATOM | 8308 | CG | GLN | F | 302 | −10.554 | 30.054 | −3.337 | 1.00 | 50.00 | C |
| ATOM | 8309 | CD | GLN | F | 302 | −11.522 | 30.367 | −4.471 | 1.00 | 50.00 | C |
| ATOM | 8310 | OE1 | GLN | F | 302 | −11.223 | 31.078 | −5.429 | 1.00 | 50.00 | O |
| ATOM | 8311 | NE2 | GLN | F | 302 | −12.727 | 29.834 | −4.398 | 1.00 | 50.00 | N |
| ATOM | 8312 | N | LEU | F | 303 | −6.720 | 30.055 | −6.405 | 1.00 | 50.00 | N |
| ATOM | 8313 | CA | LEU | F | 303 | −5.422 | 30.534 | −6.878 | 1.00 | 50.00 | C |
| ATOM | 8314 | C | LEU | F | 303 | −5.482 | 32.009 | −7.260 | 1.00 | 50.00 | C |
| ATOM | 8315 | O | LEU | F | 303 | −6.503 | 32.475 | −7.765 | 1.00 | 50.00 | O |
| ATOM | 8316 | CB | LEU | F | 303 | −4.659 | 29.735 | −7.934 | 1.00 | 50.00 | C |
| ATOM | 8317 | CG | LEU | F | 303 | −4.240 | 28.319 | −7.540 | 1.00 | 50.00 | C |
| ATOM | 8318 | CD1 | LEU | F | 303 | −5.365 | 27.288 | −7.549 | 1.00 | 50.00 | C |
| ATOM | 8319 | CD2 | LEU | F | 303 | −3.154 | 27.828 | −8.494 | 1.00 | 50.00 | C |
| ATOM | 8320 | N | PRO | F | 304 | −4.417 | 32.771 | −7.063 | 1.00 | 50.00 | N |
| ATOM | 8321 | CA | PRO | F | 304 | −4.318 | 34.162 | −7.502 | 1.00 | 50.00 | C |
| ATOM | 8322 | C | PRO | F | 304 | −3.684 | 34.328 | −8.877 | 1.00 | 50.00 | C |
| ATOM | 8323 | O | PRO | F | 304 | −3.293 | 33.347 | −9.506 | 1.00 | 50.00 | O |
| ATOM | 8324 | CB | PRO | F | 304 | −3.443 | 34.788 | −6.420 | 1.00 | 50.00 | C |
| ATOM | 8325 | CG | PRO | F | 304 | −2.481 | 33.628 | −6.194 | 1.00 | 50.00 | C |
| ATOM | 8326 | CD | PRO | F | 304 | −3.207 | 32.305 | −6.388 | 1.00 | 50.00 | C |
| ATOM | 8327 | N | LEU | F | 305 | −3.650 | 35.538 | −9.407 | 1.00 | 50.00 | N |
| ATOM | 8328 | CA | LEU | F | 305 | −3.250 | 35.729 | −10.800 | 1.00 | 50.00 | C |
| ATOM | 8329 | C | LEU | F | 305 | −2.403 | 36.987 | −10.861 | 1.00 | 50.00 | C |
| ATOM | 8330 | O | LEU | F | 305 | −2.838 | 38.134 | −10.955 | 1.00 | 50.00 | O |
| ATOM | 8331 | CB | LEU | F | 305 | −4.427 | 35.853 | −11.760 | 1.00 | 50.00 | C |
| ATOM | 8332 | CG | LEU | F | 305 | −5.172 | 34.552 | −12.047 | 1.00 | 50.00 | C |
| ATOM | 8333 | CD1 | LEU | F | 305 | −6.059 | 34.167 | −10.865 | 1.00 | 50.00 | C |
| ATOM | 8334 | CD2 | LEU | F | 305 | −6.085 | 34.915 | −13.211 | 1.00 | 50.00 | C |
| ATOM | 8335 | N | TYR | F | 306 | −1.125 | 36.663 | −10.823 | 1.00 | 50.00 | N |
| ATOM | 8336 | CA | TYR | F | 306 | −0.092 | 37.687 | −10.731 | 1.00 | 50.00 | C |
| ATOM | 8337 | C | TYR | F | 306 | 0.265 | 38.192 | −12.122 | 1.00 | 50.00 | C |
| ATOM | 8338 | O | TYR | F | 306 | 0.805 | 37.493 | −12.979 | 1.00 | 50.00 | O |
| ATOM | 8339 | CB | TYR | F | 306 | 0.967 | 37.047 | −9.839 | 1.00 | 50.00 | C |
| ATOM | 8340 | CG | TYR | F | 306 | 2.244 | 37.844 | −9.732 | 1.00 | 50.00 | C |
| ATOM | 8341 | CD1 | TYR | F | 306 | 2.185 | 38.979 | −9.010 | 1.00 | 50.00 | C |
| ATOM | 8342 | CD2 | TYR | F | 306 | 3.444 | 37.321 | −10.053 | 1.00 | 50.00 | C |
| ATOM | 8343 | CE1 | TYR | F | 306 | 3.268 | 39.336 | −8.295 | 1.00 | 50.00 | C |
| ATOM | 8344 | CE2 | TYR | F | 306 | 4.555 | 37.933 | −9.597 | 1.00 | 50.00 | C |
| ATOM | 8345 | CZ | TYR | F | 306 | 4.481 | 38.848 | −8.613 | 1.00 | 50.00 | C |
| ATOM | 8346 | OH | TYR | F | 306 | 5.560 | 39.561 | −8.165 | 1.00 | 50.00 | O |
| ATOM | 8347 | N | GLY | F | 307 | −0.077 | 39.459 | −12.271 | 1.00 | 50.00 | N |
| ATOM | 8348 | CA | GLY | F | 307 | 0.457 | 40.323 | −13.319 | 1.00 | 50.00 | C |
| ATOM | 8349 | C | GLY | F | 307 | 1.522 | 41.200 | −12.680 | 1.00 | 50.00 | C |
| ATOM | 8350 | O | GLY | F | 307 | 1.251 | 41.963 | −11.754 | 1.00 | 50.00 | O |
| ATOM | 8351 | N | VAL | F | 308 | 2.736 | 41.092 | −13.188 | 1.00 | 50.00 | N |
| ATOM | 8352 | CA | VAL | F | 308 | 3.855 | 41.943 | −12.783 | 1.00 | 50.00 | C |
| ATOM | 8353 | C | VAL | F | 308 | 3.806 | 43.362 | −13.322 | 1.00 | 50.00 | C |
| ATOM | 8354 | O | VAL | F | 308 | 3.592 | 43.573 | −14.515 | 1.00 | 50.00 | O |
| ATOM | 8355 | CB | VAL | F | 308 | 5.207 | 41.391 | −13.231 | 1.00 | 50.00 | C |
| ATOM | 8356 | CG1 | VAL | F | 308 | 5.322 | 41.166 | −14.738 | 1.00 | 50.00 | C |
| ATOM | 8357 | CG2 | VAL | F | 308 | 6.515 | 41.863 | −12.604 | 1.00 | 50.00 | C |
| ATOM | 8358 | N | ILE | F | 309 | 4.338 | 44.245 | −12.495 | 1.00 | 50.00 | N |
| ATOM | 8359 | CA | ILE | F | 309 | 4.912 | 45.501 | −12.975 | 1.00 | 50.00 | C |
| ATOM | 8360 | C | ILE | F | 309 | 6.231 | 45.209 | −13.685 | 1.00 | 50.00 | C |
| ATOM | 8361 | O | ILE | F | 309 | 7.290 | 45.270 | −13.061 | 1.00 | 50.00 | O |
| ATOM | 8362 | CB | ILE | F | 309 | 4.943 | 46.535 | −11.847 | 1.00 | 50.00 | C |
| ATOM | 8363 | CG1 | ILE | F | 309 | 3.623 | 46.705 | −11.093 | 1.00 | 50.00 | C |
| ATOM | 8364 | CG2 | ILE | F | 309 | 5.341 | 47.893 | −12.423 | 1.00 | 50.00 | C |
| ATOM | 8365 | CD1 | ILE | F | 309 | 3.572 | 47.638 | −9.889 | 1.00 | 50.00 | C |
| ATOM | 8366 | N | ASP | F | 310 | 6.153 | 44.861 | −14.961 | 1.00 | 50.00 | N |
| ATOM | 8367 | CA | ASP | F | 310 | 7.288 | 44.595 | −15.848 | 1.00 | 50.00 | C |
| ATOM | 8368 | C | ASP | F | 310 | 7.957 | 45.877 | −16.330 | 1.00 | 50.00 | C |
| ATOM | 8369 | O | ASP | F | 310 | 7.912 | 46.324 | −17.475 | 1.00 | 50.00 | O |
| ATOM | 8370 | CB | ASP | F | 310 | 6.936 | 43.673 | −17.016 | 1.00 | 50.00 | C |
| ATOM | 8371 | CG | ASP | F | 310 | 5.728 | 44.041 | −17.866 | 1.00 | 50.00 | C |
| ATOM | 8372 | OD1 | ASP | F | 310 | 4.613 | 43.581 | −17.537 | 1.00 | 50.00 | O |
| ATOM | 8373 | OD2 | ASP | F | 310 | 5.874 | 44.794 | −18.853 | 1.00 | 50.00 | O |
| ATOM | 8374 | N | THR | F | 311 | 8.617 | 46.458 | −15.346 | 1.00 | 50.00 | N |
| ATOM | 8375 | CA | THR | F | 311 | 9.511 | 47.610 | −15.430 | 1.00 | 50.00 | C |
| ATOM | 8376 | C | THR | F | 311 | 10.696 | 47.056 | −14.645 | 1.00 | 50.00 | C |
| ATOM | 8377 | O | THR | F | 311 | 10.452 | 46.567 | −13.540 | 1.00 | 50.00 | O |
| ATOM | 8378 | CB | THR | F | 311 | 8.856 | 48.863 | −14.840 | 1.00 | 50.00 | C |
| ATOM | 8379 | OG1 | THR | F | 311 | 8.704 | 48.788 | −13.430 | 1.00 | 50.00 | O |
| ATOM | 8380 | CG2 | THR | F | 311 | 7.483 | 49.237 | −15.397 | 1.00 | 50.00 | C |
| ATOM | 8381 | N | PRO | F | 312 | 11.962 | 47.086 | −15.039 | 1.00 | 50.00 | N |
| ATOM | 8382 | CA | PRO | F | 312 | 13.064 | 46.752 | −14.135 | 1.00 | 50.00 | C |
| ATOM | 8383 | C | PRO | F | 312 | 13.341 | 47.823 | −13.086 | 1.00 | 50.00 | C |
| ATOM | 8384 | O | PRO | F | 312 | 14.411 | 48.423 | −12.994 | 1.00 | 50.00 | O |
| ATOM | 8385 | CB | PRO | F | 312 | 14.196 | 46.395 | −15.096 | 1.00 | 50.00 | C |
| ATOM | 8386 | CG | PRO | F | 312 | 13.924 | 47.205 | −16.360 | 1.00 | 50.00 | C |
| ATOM | 8387 | CD | PRO | F | 312 | 12.426 | 47.500 | −16.362 | 1.00 | 50.00 | C |
| ATOM | 8388 | N | CYS | F | 313 | 12.292 | 48.111 | −12.333 | 1.00 | 50.00 | N |
| ATOM | 8389 | CA | CYS | F | 313 | 12.302 | 48.232 | −10.879 | 1.00 | 50.00 | C |
| ATOM | 8390 | C | CYS | F | 313 | 10.984 | 48.012 | −10.145 | 1.00 | 50.00 | C |
| ATOM | 8391 | O | CYS | F | 313 | 9.934 | 47.965 | −10.784 | 1.00 | 50.00 | O |
| ATOM | 8392 | CB | CYS | F | 313 | 12.846 | 49.624 | −10.585 | 1.00 | 50.00 | C |
| ATOM | 8393 | SG | CYS | F | 313 | 12.649 | 50.731 | −11.994 | 1.00 | 50.00 | S |
| ATOM | 8394 | N | TRP | F | 314 | 10.838 | 48.127 | −8.833 | 1.00 | 50.00 | N |
| ATOM | 8395 | CA | TRP | F | 314 | 11.660 | 47.616 | −7.734 | 1.00 | 50.00 | C |
| ATOM | 8396 | C | TRP | F | 314 | 11.327 | 48.478 | −6.516 | 1.00 | 50.00 | C |
| ATOM | 8397 | O | TRP | F | 314 | 11.599 | 49.677 | −6.556 | 1.00 | 50.00 | O |
| ATOM | 8398 | CB | TRP | F | 314 | 13.151 | 47.271 | −7.861 | 1.00 | 50.00 | C |
| ATOM | 8399 | CG | TRP | F | 314 | 13.708 | 46.174 | −8.749 | 1.00 | 50.00 | C |
| ATOM | 8400 | CD1 | TRP | F | 314 | 12.847 | 45.241 | −9.316 | 1.00 | 50.00 | C |
| ATOM | 8401 | CD2 | TRP | F | 314 | 14.838 | 46.086 | −9.564 | 1.00 | 50.00 | C |
| ATOM | 8402 | NE1 | TRP | F | 314 | 13.398 | 44.493 | −10.337 | 1.00 | 50.00 | N |
| ATOM | 8403 | CE2 | TRP | F | 314 | 14.644 | 45.054 | −10.491 | 1.00 | 50.00 | C |
| ATOM | 8404 | CE3 | TRP | F | 314 | 15.787 | 47.013 | −9.838 | 1.00 | 50.00 | C |
| ATOM | 8405 | CZ2 | TRP | F | 314 | 15.354 | 44.989 | −11.637 | 1.00 | 50.00 | C |
| ATOM | 8406 | CZ3 | TRP | F | 314 | 16.499 | 46.930 | −10.981 | 1.00 | 50.00 | C |
| ATOM | 8407 | CH2 | TRP | F | 314 | 16.284 | 45.936 | −11.868 | 1.00 | 50.00 | C |
| ATOM | 8408 | N | LYS | F | 315 | 10.770 | 47.943 | −5.438 | 1.00 | 50.00 | N |
| ATOM | 8409 | CA | LYS | F | 315 | 10.670 | 48.524 | −4.095 | 1.00 | 50.00 | C |
| ATOM | 8410 | C | LYS | F | 315 | 11.916 | 48.320 | −3.240 | 1.00 | 50.00 | C |
| ATOM | 8411 | O | LYS | F | 315 | 12.419 | 47.200 | −3.158 | 1.00 | 50.00 | O |
| ATOM | 8412 | CB | LYS | F | 315 | 9.545 | 47.906 | −3.262 | 1.00 | 50.00 | C |
| ATOM | 8413 | CG | LYS | F | 315 | 8.139 | 48.248 | −3.746 | 1.00 | 50.00 | C |
| ATOM | 8414 | CD | LYS | F | 315 | 6.981 | 47.656 | −2.946 | 1.00 | 50.00 | C |
| ATOM | 8415 | CE | LYS | F | 315 | 5.658 | 48.058 | −3.593 | 1.00 | 50.00 | C |
| ATOM | 8416 | NZ | LYS | F | 315 | 4.484 | 47.653 | −2.809 | 1.00 | 50.00 | N |
| ATOM | 8417 | N | LEU | F | 316 | 12.429 | 49.360 | −2.602 | 1.00 | 50.00 | N |
| ATOM | 8418 | CA | LEU | F | 316 | 13.763 | 49.360 | −1.999 | 1.00 | 50.00 | C |
| ATOM | 8419 | C | LEU | F | 316 | 13.651 | 49.860 | −0.562 | 1.00 | 50.00 | C |
| ATOM | 8420 | O | LEU | F | 316 | 13.478 | 51.055 | −0.325 | 1.00 | 50.00 | O |
| ATOM | 8421 | CB | LEU | F | 316 | 14.765 | 50.137 | −2.857 | 1.00 | 50.00 | C |
| ATOM | 8422 | CG | LEU | F | 316 | 15.283 | 49.501 | −4.150 | 1.00 | 50.00 | C |
| ATOM | 8423 | CD1 | LEU | F | 316 | 14.300 | 49.499 | −5.318 | 1.00 | 50.00 | C |
| ATOM | 8424 | CD2 | LEU | F | 316 | 16.538 | 50.164 | −4.715 | 1.00 | 50.00 | C |
| ATOM | 8425 | N | HIS | F | 317 | 13.777 | 48.964 | 0.404 | 1.00 | 50.00 | N |
| ATOM | 8426 | CA | HIS | F | 317 | 13.735 | 49.247 | 1.840 | 1.00 | 50.00 | C |
| ATOM | 8427 | C | HIS | F | 317 | 14.991 | 49.966 | 2.320 | 1.00 | 50.00 | C |
| ATOM | 8428 | O | HIS | F | 317 | 16.116 | 49.535 | 2.070 | 1.00 | 50.00 | O |
| ATOM | 8429 | CB | HIS | F | 317 | 13.497 | 47.960 | 2.632 | 1.00 | 50.00 | C |
| ATOM | 8430 | CG | HIS | F | 317 | 13.076 | 48.113 | 4.079 | 1.00 | 50.00 | C |
| ATOM | 8431 | ND1 | HIS | F | 317 | 11.770 | 47.986 | 4.511 | 1.00 | 50.00 | N |
| ATOM | 8432 | CD2 | HIS | F | 317 | 13.914 | 48.404 | 5.152 | 1.00 | 50.00 | C |
| ATOM | 8433 | CE1 | HIS | F | 317 | 11.948 | 48.232 | 5.856 | 1.00 | 50.00 | C |
| ATOM | 8434 | NE2 | HIS | F | 317 | 13.219 | 48.447 | 6.343 | 1.00 | 50.00 | N |
| ATOM | 8435 | N | THR | F | 318 | 14.762 | 51.043 | 3.052 | 1.00 | 50.00 | N |
| ATOM | 8436 | CA | THR | F | 318 | 15.784 | 51.874 | 3.690 | 1.00 | 50.00 | C |
| ATOM | 8437 | C | THR | F | 318 | 15.625 | 51.780 | 5.204 | 1.00 | 50.00 | C |
| ATOM | 8438 | O | THR | F | 318 | 14.527 | 51.903 | 5.748 | 1.00 | 50.00 | O |
| ATOM | 8439 | CB | THR | F | 318 | 15.623 | 53.299 | 3.158 | 1.00 | 50.00 | C |
| ATOM | 8440 | OG1 | THR | F | 318 | 15.969 | 53.348 | 1.781 | 1.00 | 50.00 | O |
| ATOM | 8441 | CG2 | THR | F | 318 | 16.433 | 54.392 | 3.854 | 1.00 | 50.00 | C |
| ATOM | 8442 | N | SER | F | 319 | 16.752 | 51.659 | 5.886 | 1.00 | 50.00 | N |
| ATOM | 8443 | CA | SER | F | 319 | 16.885 | 51.899 | 7.324 | 1.00 | 50.00 | C |
| ATOM | 8444 | C | SER | F | 319 | 18.035 | 52.860 | 7.608 | 1.00 | 50.00 | C |
| ATOM | 8445 | O | SER | F | 319 | 19.120 | 52.593 | 7.093 | 1.00 | 50.00 | O |
| ATOM | 8446 | CB | SER | F | 319 | 17.139 | 50.600 | 8.088 | 1.00 | 50.00 | C |
| ATOM | 8447 | OG | SER | F | 319 | 16.009 | 49.745 | 8.007 | 1.00 | 50.00 | O |
| ATOM | 8448 | N | PRO | F | 320 | 17.937 | 53.901 | 8.424 | 1.00 | 50.00 | N |
| ATOM | 8449 | CA | PRO | F | 320 | 19.071 | 54.753 | 8.781 | 1.00 | 50.00 | C |
| ATOM | 8450 | C | PRO | F | 320 | 19.687 | 54.470 | 10.146 | 1.00 | 50.00 | C |
| ATOM | 8451 | O | PRO | F | 320 | 19.003 | 54.478 | 11.170 | 1.00 | 50.00 | O |
| ATOM | 8452 | CB | PRO | F | 320 | 18.471 | 56.152 | 8.668 | 1.00 | 50.00 | C |
| ATOM | 8453 | CG | PRO | F | 320 | 17.011 | 55.988 | 9.080 | 1.00 | 50.00 | C |
| ATOM | 8454 | CD | PRO | F | 320 | 16.670 | 54.526 | 8.801 | 1.00 | 50.00 | C |
| ATOM | 8455 | N | LEU | F | 321 | 20.999 | 54.306 | 10.153 | 1.00 | 50.00 | N |
| ATOM | 8456 | CA | LEU | F | 321 | 21.810 | 54.346 | 11.370 | 1.00 | 50.00 | C |
| ATOM | 8457 | C | LEU | F | 321 | 22.123 | 55.778 | 11.782 | 1.00 | 50.00 | C |
| ATOM | 8458 | O | LEU | F | 321 | 22.156 | 56.741 | 11.016 | 1.00 | 50.00 | O |
| ATOM | 8459 | CB | LEU | F | 321 | 23.128 | 53.583 | 11.212 | 1.00 | 50.00 | C |
| ATOM | 8460 | CG | LEU | F | 321 | 23.116 | 52.069 | 11.419 | 1.00 | 50.00 | C |
| ATOM | 8461 | CD1 | LEU | F | 321 | 22.743 | 51.688 | 12.850 | 1.00 | 50.00 | C |
| ATOM | 8462 | CD2 | LEU | F | 321 | 22.254 | 51.332 | 10.396 | 1.00 | 50.00 | C |
| ATOM | 8463 | N | CYS | F | 322 | 22.454 | 55.830 | 13.060 | 1.00 | 50.00 | N |
| ATOM | 8464 | CA | CYS | F | 322 | 23.032 | 56.996 | 13.719 | 1.00 | 50.00 | C |
| ATOM | 8465 | C | CYS | F | 322 | 24.552 | 57.084 | 13.603 | 1.00 | 50.00 | C |
| ATOM | 8466 | O | CYS | F | 322 | 25.279 | 56.604 | 14.472 | 1.00 | 50.00 | O |
| ATOM | 8467 | CB | CYS | F | 322 | 22.347 | 56.858 | 15.079 | 1.00 | 50.00 | C |
| ATOM | 8468 | SG | CYS | F | 322 | 20.674 | 57.519 | 14.947 | 1.00 | 50.00 | S |
| ATOM | 8469 | N | THR | F | 323 | 25.025 | 57.802 | 12.595 | 1.00 | 50.00 | N |
| ATOM | 8470 | CA | THR | F | 323 | 26.386 | 58.325 | 12.431 | 1.00 | 50.00 | C |
| ATOM | 8471 | C | THR | F | 323 | 26.719 | 59.696 | 13.014 | 1.00 | 50.00 | C |
| ATOM | 8472 | O | THR | F | 323 | 25.932 | 60.631 | 12.895 | 1.00 | 50.00 | O |
| ATOM | 8473 | CB | THR | F | 323 | 26.603 | 58.468 | 10.923 | 1.00 | 50.00 | C |
| ATOM | 8474 | OG1 | THR | F | 323 | 26.633 | 57.165 | 10.359 | 1.00 | 50.00 | O |
| ATOM | 8475 | CG2 | THR | F | 323 | 27.810 | 59.244 | 10.391 | 1.00 | 50.00 | C |
| ATOM | 8476 | N | THR | F | 324 | 27.959 | 60.036 | 13.328 | 1.00 | 50.00 | N |
| ATOM | 8477 | CA | THR | F | 324 | 28.370 | 61.428 | 13.534 | 1.00 | 50.00 | C |
| ATOM | 8478 | C | THR | F | 324 | 28.297 | 62.338 | 12.310 | 1.00 | 50.00 | C |
| ATOM | 8479 | O | THR | F | 324 | 27.203 | 62.824 | 12.027 | 1.00 | 50.00 | O |
| ATOM | 8480 | CB | THR | F | 324 | 29.592 | 61.445 | 14.454 | 1.00 | 50.00 | C |
| ATOM | 8481 | OG1 | THR | F | 324 | 29.311 | 60.731 | 15.650 | 1.00 | 50.00 | O |
| ATOM | 8482 | CG2 | THR | F | 324 | 30.033 | 62.837 | 14.900 | 1.00 | 50.00 | C |
| ATOM | 8483 | N | ASN | F | 325 | 29.368 | 62.623 | 11.586 | 1.00 | 50.00 | N |
| ATOM | 8484 | CA | ASN | F | 325 | 29.414 | 63.603 | 10.497 | 1.00 | 50.00 | C |
| ATOM | 8485 | C | ASN | F | 325 | 28.969 | 65.008 | 10.899 | 1.00 | 50.00 | C |
| ATOM | 8486 | O | ASN | F | 325 | 29.748 | 65.735 | 11.514 | 1.00 | 50.00 | O |
| ATOM | 8487 | CB | ASN | F | 325 | 28.869 | 63.071 | 9.169 | 1.00 | 50.00 | C |
| ATOM | 8488 | CG | ASN | F | 325 | 29.522 | 61.898 | 8.450 | 1.00 | 50.00 | C |
| ATOM | 8489 | OD1 | ASN | F | 325 | 28.840 | 61.122 | 7.781 | 1.00 | 50.00 | O |
| ATOM | 8490 | ND2 | ASN | F | 325 | 30.832 | 61.740 | 8.490 | 1.00 | 50.00 | N |
| ATOM | 8491 | N | THR | F | 326 | 27.729 | 65.375 | 10.618 | 1.00 | 50.00 | N |
| ATOM | 8492 | CA | THR | F | 326 | 27.010 | 66.484 | 11.245 | 1.00 | 50.00 | C |
| ATOM | 8493 | C | THR | F | 326 | 26.022 | 65.874 | 12.234 | 1.00 | 50.00 | C |
| ATOM | 8494 | O | THR | F | 326 | 25.170 | 65.071 | 11.855 | 1.00 | 50.00 | O |
| ATOM | 8495 | CB | THR | F | 326 | 26.351 | 67.327 | 10.151 | 1.00 | 50.00 | C |
| ATOM | 8496 | OG1 | THR | F | 326 | 27.349 | 67.919 | 9.331 | 1.00 | 50.00 | O |
| ATOM | 8497 | CG2 | THR | F | 326 | 25.451 | 68.461 | 10.642 | 1.00 | 50.00 | C |
| ATOM | 8498 | N | LYS | F | 327 | 26.134 | 66.242 | 13.501 | 1.00 | 50.00 | N |
| ATOM | 8499 | CA | LYS | F | 327 | 25.382 | 65.650 | 14.609 | 1.00 | 50.00 | C |
| ATOM | 8500 | C | LYS | F | 327 | 25.025 | 66.707 | 15.650 | 1.00 | 50.00 | C |
| ATOM | 8501 | O | LYS | F | 327 | 25.697 | 67.732 | 15.768 | 1.00 | 50.00 | O |
| ATOM | 8502 | CB | LYS | F | 327 | 26.250 | 64.534 | 15.194 | 1.00 | 50.00 | C |
| ATOM | 8503 | CG | LYS | F | 327 | 25.541 | 63.506 | 16.073 | 1.00 | 50.00 | C |
| ATOM | 8504 | CD | LYS | F | 327 | 26.427 | 62.485 | 16.784 | 1.00 | 50.00 | C |
| ATOM | 8505 | CE | LYS | F | 327 | 25.566 | 61.448 | 17.498 | 1.00 | 50.00 | C |
| ATOM | 8506 | NZ | LYS | F | 327 | 26.210 | 60.464 | 18.380 | 1.00 | 50.00 | N |
| ATOM | 8507 | N | GLU | F | 328 | 23.999 | 66.466 | 16.452 | 1.00 | 50.00 | N |
| ATOM | 8508 | CA | GLU | F | 328 | 23.711 | 67.232 | 17.667 | 1.00 | 50.00 | C |
| ATOM | 8509 | C | GLU | F | 328 | 23.474 | 66.232 | 18.794 | 1.00 | 50.00 | C |
| ATOM | 8510 | O | GLU | F | 328 | 22.460 | 65.536 | 18.800 | 1.00 | 50.00 | O |
| ATOM | 8511 | CB | GLU | F | 328 | 22.558 | 68.224 | 17.495 | 1.00 | 50.00 | C |
| ATOM | 8512 | CG | GLU | F | 328 | 22.641 | 69.257 | 16.372 | 1.00 | 50.00 | C |
| ATOM | 8513 | CD | GLU | F | 328 | 23.478 | 70.472 | 16.742 | 1.00 | 50.00 | C |
| ATOM | 8514 | OE1 | GLU | F | 328 | 24.720 | 70.418 | 16.609 | 1.00 | 50.00 | O |
| ATOM | 8515 | OE2 | GLU | F | 328 | 22.898 | 71.487 | 17.184 | 1.00 | 50.00 | O |
| ATOM | 8516 | N | GLY | F | 329 | 24.373 | 66.083 | 19.754 | 1.00 | 50.00 | N |
| ATOM | 8517 | CA | GLY | F | 329 | 24.229 | 65.124 | 20.850 | 1.00 | 50.00 | C |
| ATOM | 8518 | C | GLY | F | 329 | 24.341 | 63.651 | 20.478 | 1.00 | 50.00 | C |
| ATOM | 8519 | O | GLY | F | 329 | 25.348 | 63.278 | 19.877 | 1.00 | 50.00 | O |
| ATOM | 8520 | N | SER | F | 330 | 23.346 | 62.843 | 20.812 | 1.00 | 50.00 | N |
| ATOM | 8521 | CA | SER | F | 330 | 23.121 | 61.524 | 20.214 | 1.00 | 50.00 | C |
| ATOM | 8522 | C | SER | F | 330 | 22.218 | 61.471 | 18.985 | 1.00 | 50.00 | C |
| ATOM | 8523 | O | SER | F | 330 | 21.794 | 60.384 | 18.594 | 1.00 | 50.00 | O |
| ATOM | 8524 | CB | SER | F | 330 | 22.600 | 60.549 | 21.269 | 1.00 | 50.00 | C |
| ATOM | 8525 | OG | SER | F | 330 | 23.559 | 60.315 | 22.290 | 1.00 | 50.00 | O |
| ATOM | 8526 | N | ASN | F | 331 | 21.939 | 62.585 | 18.327 | 1.00 | 50.00 | N |
| ATOM | 8527 | CA | ASN | F | 331 | 21.039 | 62.660 | 17.178 | 1.00 | 50.00 | C |
| ATOM | 8528 | C | ASN | F | 331 | 21.832 | 62.895 | 15.902 | 1.00 | 50.00 | C |
| ATOM | 8529 | O | ASN | F | 331 | 22.621 | 63.831 | 15.782 | 1.00 | 50.00 | O |
| ATOM | 8530 | CB | ASN | F | 331 | 19.927 | 63.687 | 17.381 | 1.00 | 50.00 | C |
| ATOM | 8531 | CG | ASN | F | 331 | 20.087 | 65.200 | 17.347 | 1.00 | 50.00 | C |
| ATOM | 8532 | OD1 | ASN | F | 331 | 19.714 | 65.895 | 18.291 | 1.00 | 50.00 | O |
| ATOM | 8533 | ND2 | ASN | F | 331 | 20.597 | 65.766 | 16.268 | 1.00 | 50.00 | N |
| ATOM | 8534 | N | ILE | F | 332 | 21.568 | 62.048 | 14.923 | 1.00 | 50.00 | N |
| ATOM | 8535 | CA | ILE | F | 332 | 22.300 | 62.035 | 13.657 | 1.00 | 50.00 | C |
| ATOM | 8536 | C | ILE | F | 332 | 21.191 | 62.671 | 12.836 | 1.00 | 50.00 | C |
| ATOM | 8537 | O | ILE | F | 332 | 21.052 | 63.883 | 12.996 | 1.00 | 50.00 | O |
| ATOM | 8538 | CB | ILE | F | 332 | 22.939 | 60.700 | 13.276 | 1.00 | 50.00 | C |
| ATOM | 8539 | CG1 | ILE | F | 332 | 23.793 | 60.259 | 14.468 | 1.00 | 50.00 | C |
| ATOM | 8540 | CG2 | ILE | F | 332 | 23.467 | 60.746 | 11.839 | 1.00 | 50.00 | C |
| ATOM | 8541 | CD1 | ILE | F | 332 | 23.193 | 59.796 | 15.795 | 1.00 | 50.00 | C |
| ATOM | 8542 | N | CYS | F | 333 | 20.331 | 62.101 | 12.011 | 1.00 | 50.00 | N |
| ATOM | 8543 | CA | CYS | F | 333 | 20.119 | 60.692 | 11.676 | 1.00 | 50.00 | C |
| ATOM | 8544 | C | CYS | F | 333 | 19.072 | 60.897 | 10.586 | 1.00 | 50.00 | C |
| ATOM | 8545 | O | CYS | F | 333 | 18.025 | 61.517 | 10.773 | 1.00 | 50.00 | O |
| ATOM | 8546 | CB | CYS | F | 333 | 19.567 | 59.711 | 12.711 | 1.00 | 50.00 | C |
| ATOM | 8547 | SG | CYS | F | 333 | 20.366 | 59.394 | 14.298 | 1.00 | 50.00 | S |
| ATOM | 8548 | N | LEU | F | 334 | 19.372 | 60.391 | 9.403 | 1.00 | 50.00 | N |
| ATOM | 8549 | CA | LEU | F | 334 | 18.859 | 60.960 | 8.157 | 1.00 | 50.00 | C |
| ATOM | 8550 | C | LEU | F | 334 | 17.363 | 60.796 | 7.929 | 1.00 | 50.00 | C |
| ATOM | 8551 | O | LEU | F | 334 | 16.751 | 59.820 | 8.362 | 1.00 | 50.00 | O |
| ATOM | 8552 | CB | LEU | F | 334 | 19.674 | 60.370 | 7.007 | 1.00 | 50.00 | C |
| ATOM | 8553 | CG | LEU | F | 334 | 21.029 | 61.058 | 6.857 | 1.00 | 50.00 | C |
| ATOM | 8554 | CD1 | LEU | F | 334 | 22.066 | 60.732 | 7.932 | 1.00 | 50.00 | C |
| ATOM | 8555 | CD2 | LEU | F | 334 | 21.608 | 60.697 | 5.496 | 1.00 | 50.00 | C |
| ATOM | 8556 | N | THR | F | 335 | 16.808 | 61.789 | 7.255 | 1.00 | 50.00 | N |
| ATOM | 8557 | CA | THR | F | 335 | 15.373 | 61.863 | 6.981 | 1.00 | 50.00 | C |
| ATOM | 8558 | C | THR | F | 335 | 15.165 | 61.317 | 5.575 | 1.00 | 50.00 | C |
| ATOM | 8559 | O | THR | F | 335 | 15.345 | 62.008 | 4.573 | 1.00 | 50.00 | O |
| ATOM | 8560 | CB | THR | F | 335 | 14.811 | 63.273 | 7.155 | 1.00 | 50.00 | C |
| ATOM | 8561 | OG1 | THR | F | 335 | 14.989 | 63.681 | 8.504 | 1.00 | 50.00 | O |
| ATOM | 8562 | CG2 | THR | F | 335 | 13.324 | 63.373 | 6.821 | 1.00 | 50.00 | C |
| ATOM | 8563 | N | ARG | F | 336 | 14.753 | 60.062 | 5.566 | 1.00 | 50.00 | N |
| ATOM | 8564 | CA | ARG | F | 336 | 14.296 | 59.362 | 4.367 | 1.00 | 50.00 | C |
| ATOM | 8565 | C | ARG | F | 336 | 13.204 | 58.398 | 4.812 | 1.00 | 50.00 | C |
| ATOM | 8566 | O | ARG | F | 336 | 12.952 | 58.105 | 5.980 | 1.00 | 50.00 | O |
| ATOM | 8567 | CB | ARG | F | 336 | 15.425 | 58.645 | 3.626 | 1.00 | 50.00 | C |
| ATOM | 8568 | CG | ARG | F | 336 | 16.532 | 59.604 | 3.197 | 1.00 | 50.00 | C |
| ATOM | 8569 | CD | ARG | F | 336 | 17.548 | 58.956 | 2.265 | 1.00 | 50.00 | C |
| ATOM | 8570 | NE | ARG | F | 336 | 18.546 | 59.981 | 1.963 | 1.00 | 50.00 | N |
| ATOM | 8571 | CZ | ARG | F | 336 | 19.511 | 59.825 | 1.073 | 1.00 | 50.00 | C |
| ATOM | 8572 | NH1 | ARG | F | 336 | 19.523 | 58.900 | 0.137 | 1.00 | 50.00 | N |
| ATOM | 8573 | NH2 | ARG | F | 336 | 20.637 | 60.503 | 1.151 | 1.00 | 50.00 | N |
| ATOM | 8574 | N | THR | F | 337 | 12.540 | 57.881 | 3.794 | 1.00 | 50.00 | N |
| ATOM | 8575 | CA | THR | F | 337 | 11.415 | 56.966 | 3.963 | 1.00 | 50.00 | C |
| ATOM | 8576 | C | THR | F | 337 | 11.848 | 55.538 | 4.272 | 1.00 | 50.00 | C |
| ATOM | 8577 | O | THR | F | 337 | 12.990 | 55.144 | 4.039 | 1.00 | 50.00 | O |
| ATOM | 8578 | CB | THR | F | 337 | 10.561 | 57.108 | 2.704 | 1.00 | 50.00 | C |
| ATOM | 8579 | OG1 | THR | F | 337 | 11.254 | 56.762 | 1.512 | 1.00 | 50.00 | O |
| ATOM | 8580 | CG2 | THR | F | 337 | 9.869 | 58.462 | 2.553 | 1.00 | 50.00 | C |
| ATOM | 8581 | N | ASP | F | 338 | 10.920 | 54.758 | 4.802 | 1.00 | 50.00 | N |
| ATOM | 8582 | CA | ASP | F | 338 | 11.116 | 53.333 | 5.071 | 1.00 | 50.00 | C |
| ATOM | 8583 | C | ASP | F | 338 | 11.226 | 52.435 | 3.843 | 1.00 | 50.00 | C |
| ATOM | 8584 | O | ASP | F | 338 | 12.008 | 51.486 | 3.888 | 1.00 | 50.00 | O |
| ATOM | 8585 | CB | ASP | F | 338 | 10.033 | 52.816 | 6.019 | 1.00 | 50.00 | C |
| ATOM | 8586 | CG | ASP | F | 338 | 10.067 | 53.374 | 7.435 | 1.00 | 50.00 | C |
| ATOM | 8587 | OD1 | ASP | F | 338 | 11.095 | 53.212 | 8.127 | 1.00 | 50.00 | O |
| ATOM | 8588 | OD2 | ASP | F | 338 | 9.065 | 53.985 | 7.868 | 1.00 | 50.00 | O |
| ATOM | 8589 | N | ARG | F | 339 | 10.454 | 52.669 | 2.794 | 1.00 | 50.00 | N |
| ATOM | 8590 | CA | ARG | F | 339 | 10.446 | 51.878 | 1.562 | 1.00 | 50.00 | C |
| ATOM | 8591 | C | ARG | F | 339 | 10.333 | 52.845 | 0.389 | 1.00 | 50.00 | C |
| ATOM | 8592 | O | ARG | F | 339 | 9.568 | 53.804 | 0.476 | 1.00 | 50.00 | O |
| ATOM | 8593 | CB | ARG | F | 339 | 9.333 | 50.827 | 1.585 | 1.00 | 50.00 | C |
| ATOM | 8594 | CG | ARG | F | 339 | 9.413 | 49.831 | 2.742 | 1.00 | 50.00 | C |
| ATOM | 8595 | CD | ARG | F | 339 | 8.382 | 48.705 | 2.772 | 1.00 | 50.00 | C |
| ATOM | 8596 | NE | ARG | F | 339 | 8.464 | 47.991 | 4.046 | 1.00 | 50.00 | N |
| ATOM | 8597 | CZ | ARG | F | 339 | 7.656 | 47.018 | 4.436 | 1.00 | 50.00 | C |
| ATOM | 8598 | NH1 | ARG | F | 339 | 6.694 | 46.509 | 3.687 | 1.00 | 50.00 | N |
| ATOM | 8599 | NH2 | ARG | F | 339 | 7.815 | 46.522 | 5.649 | 1.00 | 50.00 | N |
| ATOM | 8600 | N | GLY | F | 340 | 11.065 | 52.638 | −0.693 | 1.00 | 50.00 | N |
| ATOM | 8601 | CA | GLY | F | 340 | 11.106 | 53.521 | −1.860 | 1.00 | 50.00 | C |
| ATOM | 8602 | C | GLY | F | 340 | 11.043 | 52.752 | −3.173 | 1.00 | 50.00 | C |
| ATOM | 8603 | O | GLY | F | 340 | 12.043 | 52.147 | −3.557 | 1.00 | 50.00 | O |
| ATOM | 8604 | N | TRP | F | 341 | 9.930 | 52.800 | −3.887 | 1.00 | 50.00 | N |
| ATOM | 8605 | CA | TRP | F | 341 | 9.765 | 52.249 | −5.233 | 1.00 | 50.00 | C |
| ATOM | 8606 | C | TRP | F | 341 | 10.297 | 53.262 | −6.237 | 1.00 | 50.00 | C |
| ATOM | 8607 | O | TRP | F | 341 | 10.086 | 54.466 | −6.096 | 1.00 | 50.00 | O |
| ATOM | 8608 | CB | TRP | F | 341 | 8.317 | 51.867 | −5.552 | 1.00 | 50.00 | C |
| ATOM | 8609 | CG | TRP | F | 341 | 8.041 | 51.139 | −6.852 | 1.00 | 50.00 | C |
| ATOM | 8610 | CD1 | TRP | F | 341 | 8.222 | 49.778 | −7.080 | 1.00 | 50.00 | C |
| ATOM | 8611 | CD2 | TRP | F | 341 | 7.588 | 51.631 | −8.071 | 1.00 | 50.00 | C |
| ATOM | 8612 | NE1 | TRP | F | 341 | 7.930 | 49.394 | −8.376 | 1.00 | 50.00 | N |
| ATOM | 8613 | CE2 | TRP | F | 341 | 7.545 | 50.572 | −8.982 | 1.00 | 50.00 | C |
| ATOM | 8614 | CE3 | TRP | F | 341 | 7.220 | 52.871 | −8.468 | 1.00 | 50.00 | C |
| ATOM | 8615 | CZ2 | TRP | F | 341 | 7.150 | 50.789 | −10.257 | 1.00 | 50.00 | C |
| ATOM | 8616 | CZ3 | TRP | F | 341 | 6.807 | 53.055 | −9.739 | 1.00 | 50.00 | C |
| ATOM | 8617 | CH2 | TRP | F | 341 | 6.768 | 52.031 | −10.617 | 1.00 | 50.00 | C |
| ATOM | 8618 | N | TYR | F | 342 | 10.952 | 52.756 | −7.269 | 1.00 | 50.00 | N |
| ATOM | 8619 | CA | TYR | F | 342 | 11.393 | 53.577 | −8.395 | 1.00 | 50.00 | C |
| ATOM | 8620 | C | TYR | F | 342 | 10.919 | 52.972 | −9.712 | 1.00 | 50.00 | C |
| ATOM | 8621 | O | TYR | F | 342 | 10.768 | 51.754 | −9.805 | 1.00 | 50.00 | O |
| ATOM | 8622 | CB | TYR | F | 342 | 12.836 | 54.050 | −8.181 | 1.00 | 50.00 | C |
| ATOM | 8623 | CG | TYR | F | 342 | 14.053 | 53.577 | −8.951 | 1.00 | 50.00 | C |
| ATOM | 8624 | CD1 | TYR | F | 342 | 14.065 | 52.343 | −9.491 | 1.00 | 50.00 | C |
| ATOM | 8625 | CD2 | TYR | F | 342 | 15.096 | 54.395 | −9.206 | 1.00 | 50.00 | C |
| ATOM | 8626 | CE1 | TYR | F | 342 | 14.960 | 52.019 | −10.447 | 1.00 | 50.00 | C |
| ATOM | 8627 | CE2 | TYR | F | 342 | 16.061 | 54.036 | −10.077 | 1.00 | 50.00 | C |
| ATOM | 8628 | CZ | TYR | F | 342 | 15.969 | 52.863 | −10.733 | 1.00 | 50.00 | C |
| ATOM | 8629 | OH | TYR | F | 342 | 16.804 | 52.534 | −11.769 | 1.00 | 50.00 | O |
| ATOM | 8630 | N | CYS | F | 343 | 10.715 | 53.756 | −10.761 | 1.00 | 50.00 | N |
| ATOM | 8631 | CA | CYS | F | 343 | 10.910 | 53.267 | −12.126 | 1.00 | 50.00 | C |
| ATOM | 8632 | C | CYS | F | 343 | 11.406 | 54.197 | −13.226 | 1.00 | 50.00 | C |
| ATOM | 8633 | O | CYS | F | 343 | 11.321 | 55.422 | −13.143 | 1.00 | 50.00 | O |
| ATOM | 8634 | CB | CYS | F | 343 | 9.913 | 52.202 | −12.586 | 1.00 | 50.00 | C |
| ATOM | 8635 | SG | CYS | F | 343 | 10.838 | 50.680 | −12.848 | 1.00 | 50.00 | S |
| ATOM | 8636 | N | ASP | F | 344 | 11.999 | 53.572 | −14.231 | 1.00 | 50.00 | N |
| ATOM | 8637 | CA | ASP | F | 344 | 12.869 | 54.211 | −15.219 | 1.00 | 50.00 | C |
| ATOM | 8638 | C | ASP | F | 344 | 12.024 | 54.577 | −16.432 | 1.00 | 50.00 | C |
| ATOM | 8639 | O | ASP | F | 344 | 11.801 | 53.777 | −17.340 | 1.00 | 50.00 | O |
| ATOM | 8640 | CB | ASP | F | 344 | 14.023 | 53.281 | −15.594 | 1.00 | 50.00 | C |
| ATOM | 8641 | CG | ASP | F | 344 | 15.023 | 52.965 | −14.491 | 1.00 | 50.00 | C |
| ATOM | 8642 | OD1 | ASP | F | 344 | 15.638 | 53.909 | −13.948 | 1.00 | 50.00 | O |
| ATOM | 8643 | OD2 | ASP | F | 344 | 15.200 | 51.773 | −14.158 | 1.00 | 50.00 | O |
| ATOM | 8644 | N | ASN | F | 345 | 11.547 | 55.810 | −16.413 | 1.00 | 50.00 | N |
| ATOM | 8645 | CA | ASN | F | 345 | 10.783 | 56.412 | −17.507 | 1.00 | 50.00 | C |
| ATOM | 8646 | C | ASN | F | 345 | 11.681 | 57.196 | −18.458 | 1.00 | 50.00 | C |
| ATOM | 8647 | O | ASN | F | 345 | 11.566 | 57.022 | −19.670 | 1.00 | 50.00 | O |
| ATOM | 8648 | CB | ASN | F | 345 | 9.601 | 57.202 | −16.942 | 1.00 | 50.00 | C |
| ATOM | 8649 | CG | ASN | F | 345 | 8.573 | 56.379 | −16.176 | 1.00 | 50.00 | C |
| ATOM | 8650 | OD1 | ASN | F | 345 | 7.856 | 55.560 | −16.748 | 1.00 | 50.00 | O |
| ATOM | 8651 | ND2 | ASN | F | 345 | 8.495 | 56.526 | −14.866 | 1.00 | 50.00 | N |
| ATOM | 8652 | N | ALA | F | 346 | 12.583 | 58.019 | −17.948 | 1.00 | 50.00 | N |
| ATOM | 8653 | CA | ALA | F | 346 | 13.741 | 58.551 | −18.667 | 1.00 | 50.00 | C |
| ATOM | 8654 | C | ALA | F | 346 | 14.837 | 58.830 | −17.645 | 1.00 | 50.00 | C |
| ATOM | 8655 | O | ALA | F | 346 | 15.836 | 58.111 | −17.624 | 1.00 | 50.00 | O |
| ATOM | 8656 | CB | ALA | F | 346 | 13.373 | 59.741 | −19.552 | 1.00 | 50.00 | C |
| ATOM | 8657 | N | GLY | F | 347 | 14.630 | 59.765 | −16.731 | 1.00 | 50.00 | N |
| ATOM | 8658 | CA | GLY | F | 347 | 15.179 | 59.675 | −15.379 | 1.00 | 50.00 | C |
| ATOM | 8659 | C | GLY | F | 347 | 14.548 | 58.606 | −14.496 | 1.00 | 50.00 | C |
| ATOM | 8660 | O | GLY | F | 347 | 13.686 | 57.826 | −14.900 | 1.00 | 50.00 | O |
| ATOM | 8661 | N | SER | F | 348 | 15.010 | 58.588 | −13.258 | 1.00 | 50.00 | N |
| ATOM | 8662 | CA | SER | F | 348 | 14.568 | 57.631 | −12.244 | 1.00 | 50.00 | C |
| ATOM | 8663 | C | SER | F | 348 | 13.518 | 58.284 | −11.353 | 1.00 | 50.00 | C |
| ATOM | 8664 | O | SER | F | 348 | 13.783 | 59.269 | −10.665 | 1.00 | 50.00 | O |
| ATOM | 8665 | CB | SER | F | 348 | 15.796 | 57.188 | −11.452 | 1.00 | 50.00 | C |
| ATOM | 8666 | OG | SER | F | 348 | 16.633 | 56.360 | −12.248 | 1.00 | 50.00 | O |
| ATOM | 8667 | N | VAL | F | 349 | 12.317 | 57.731 | −11.391 | 1.00 | 50.00 | N |
| ATOM | 8668 | CA | VAL | F | 349 | 11.127 | 58.296 | −10.754 | 1.00 | 50.00 | C |
| ATOM | 8669 | C | VAL | F | 349 | 10.965 | 57.567 | −9.426 | 1.00 | 50.00 | C |
| ATOM | 8670 | O | VAL | F | 349 | 10.689 | 56.369 | −9.453 | 1.00 | 50.00 | O |
| ATOM | 8671 | CB | VAL | F | 349 | 9.955 | 58.183 | −11.733 | 1.00 | 50.00 | C |
| ATOM | 8672 | CG1 | VAL | F | 349 | 8.583 | 58.355 | −11.085 | 1.00 | 50.00 | C |
| ATOM | 8673 | CG2 | VAL | F | 349 | 10.053 | 59.213 | −12.857 | 1.00 | 50.00 | C |
| ATOM | 8674 | N | SER | F | 350 | 11.127 | 58.238 | −8.297 | 1.00 | 50.00 | N |
| ATOM | 8675 | CA | SER | F | 350 | 11.466 | 57.603 | −7.023 | 1.00 | 50.00 | C |
| ATOM | 8676 | C | SER | F | 350 | 10.517 | 58.067 | −5.925 | 1.00 | 50.00 | C |
| ATOM | 8677 | O | SER | F | 350 | 10.778 | 59.049 | −5.232 | 1.00 | 50.00 | O |
| ATOM | 8678 | CB | SER | F | 350 | 12.935 | 57.901 | −6.718 | 1.00 | 50.00 | C |
| ATOM | 8679 | OG | SER | F | 350 | 13.812 | 57.245 | −7.622 | 1.00 | 50.00 | O |
| ATOM | 8680 | N | PHE | F | 351 | 9.407 | 57.369 | −5.747 | 1.00 | 50.00 | N |
| ATOM | 8681 | CA | PHE | F | 351 | 8.326 | 57.756 | −4.838 | 1.00 | 50.00 | C |
| ATOM | 8682 | C | PHE | F | 351 | 8.221 | 56.864 | −3.605 | 1.00 | 50.00 | C |
| ATOM | 8683 | O | PHE | F | 351 | 8.854 | 55.809 | −3.565 | 1.00 | 50.00 | O |
| ATOM | 8684 | CB | PHE | F | 351 | 7.053 | 57.855 | −5.684 | 1.00 | 50.00 | C |
| ATOM | 8685 | CG | PHE | F | 351 | 6.978 | 58.995 | −6.679 | 1.00 | 50.00 | C |
| ATOM | 8686 | CD1 | PHE | F | 351 | 7.063 | 60.254 | −6.202 | 1.00 | 50.00 | C |
| ATOM | 8687 | CD2 | PHE | F | 351 | 6.834 | 58.810 | −8.008 | 1.00 | 50.00 | C |
| ATOM | 8688 | CE1 | PHE | F | 351 | 6.994 | 61.316 | −7.029 | 1.00 | 50.00 | C |
| ATOM | 8689 | CE2 | PHE | F | 351 | 6.727 | 59.869 | −8.836 | 1.00 | 50.00 | C |
| ATOM | 8690 | CZ | PHE | F | 351 | 6.829 | 61.123 | −8.352 | 1.00 | 50.00 | C |
| ATOM | 8691 | N | PHE | F | 352 | 7.473 | 57.258 | −2.585 | 1.00 | 50.00 | N |
| ATOM | 8692 | CA | PHE | F | 352 | 7.268 | 56.495 | −1.351 | 1.00 | 50.00 | C |
| ATOM | 8693 | C | PHE | F | 352 | 6.140 | 55.484 | −1.523 | 1.00 | 50.00 | C |
| ATOM | 8694 | O | PHE | F | 352 | 4.997 | 55.904 | −1.350 | 1.00 | 50.00 | O |
| ATOM | 8695 | CB | PHE | F | 352 | 6.991 | 57.287 | −0.070 | 1.00 | 50.00 | C |
| ATOM | 8696 | CG | PHE | F | 352 | 6.696 | 56.524 | 1.206 | 1.00 | 50.00 | C |
| ATOM | 8697 | CD1 | PHE | F | 352 | 7.686 | 55.752 | 1.695 | 1.00 | 50.00 | C |
| ATOM | 8698 | CD2 | PHE | F | 352 | 5.505 | 56.537 | 1.839 | 1.00 | 50.00 | C |
| ATOM | 8699 | CE1 | PHE | F | 352 | 7.516 | 55.030 | 2.820 | 1.00 | 50.00 | C |
| ATOM | 8700 | CE2 | PHE | F | 352 | 5.323 | 55.813 | 2.963 | 1.00 | 50.00 | C |
| ATOM | 8701 | CZ | PHE | F | 352 | 6.329 | 55.065 | 3.458 | 1.00 | 50.00 | C |
| ATOM | 8702 | N | PRO | F | 353 | 6.335 | 54.184 | −1.682 | 1.00 | 50.00 | N |
| ATOM | 8703 | CA | PRO | F | 353 | 5.296 | 53.160 | −1.600 | 1.00 | 50.00 | C |
| ATOM | 8704 | C | PRO | F | 353 | 4.779 | 52.996 | −0.176 | 1.00 | 50.00 | C |
| ATOM | 8705 | O | PRO | F | 353 | 5.503 | 52.752 | 0.789 | 1.00 | 50.00 | O |
| ATOM | 8706 | CB | PRO | F | 353 | 5.999 | 51.904 | −2.114 | 1.00 | 50.00 | C |
| ATOM | 8707 | CG | PRO | F | 353 | 7.456 | 52.091 | −1.698 | 1.00 | 50.00 | C |
| ATOM | 8708 | CD | PRO | F | 353 | 7.617 | 53.574 | −2.015 | 1.00 | 50.00 | C |
| ATOM | 8709 | N | GLN | F | 354 | 3.465 | 53.103 | −0.107 | 1.00 | 50.00 | N |
| ATOM | 8710 | CA | GLN | F | 354 | 2.629 | 52.622 | 0.990 | 1.00 | 50.00 | C |
| ATOM | 8711 | C | GLN | F | 354 | 2.413 | 51.126 | 0.792 | 1.00 | 50.00 | C |
| ATOM | 8712 | O | GLN | F | 354 | 1.453 | 50.683 | 0.163 | 1.00 | 50.00 | O |
| ATOM | 8713 | CB | GLN | F | 354 | 1.325 | 53.424 | 1.002 | 1.00 | 50.00 | C |
| ATOM | 8714 | CG | GLN | F | 354 | 1.447 | 54.920 | 1.291 | 1.00 | 50.00 | C |
| ATOM | 8715 | CD | GLN | F | 354 | 1.743 | 55.287 | 2.738 | 1.00 | 50.00 | C |
| ATOM | 8716 | OE1 | GLN | F | 354 | 1.911 | 54.448 | 3.622 | 1.00 | 50.00 | O |
| ATOM | 8717 | NE2 | GLN | F | 354 | 1.843 | 56.570 | 3.034 | 1.00 | 50.00 | N |
| ATOM | 8718 | N | ALA | F | 355 | 3.331 | 50.354 | 1.351 | 1.00 | 50.00 | N |
| ATOM | 8719 | CA | ALA | F | 355 | 3.244 | 48.895 | 1.427 | 1.00 | 50.00 | C |
| ATOM | 8720 | C | ALA | F | 355 | 2.143 | 48.258 | 2.269 | 1.00 | 50.00 | C |
| ATOM | 8721 | O | ALA | F | 355 | 2.025 | 47.033 | 2.247 | 1.00 | 50.00 | O |
| ATOM | 8722 | CB | ALA | F | 355 | 4.599 | 48.410 | 1.936 | 1.00 | 50.00 | C |
| ATOM | 8723 | N | GLU | F | 356 | 1.312 | 49.029 | 2.951 | 1.00 | 50.00 | N |
| ATOM | 8724 | CA | GLU | F | 356 | 0.052 | 48.597 | 3.560 | 1.00 | 50.00 | C |
| ATOM | 8725 | C | GLU | F | 356 | −0.887 | 47.724 | 2.735 | 1.00 | 50.00 | C |
| ATOM | 8726 | O | GLU | F | 356 | −1.527 | 46.841 | 3.305 | 1.00 | 50.00 | O |
| ATOM | 8727 | CB | GLU | F | 356 | −0.689 | 49.831 | 4.079 | 1.00 | 50.00 | C |
| ATOM | 8728 | CG | GLU | F | 356 | −0.035 | 50.621 | 5.212 | 1.00 | 50.00 | C |
| ATOM | 8729 | CD | GLU | F | 356 | 0.053 | 49.865 | 6.529 | 1.00 | 50.00 | C |
| ATOM | 8730 | OE1 | GLU | F | 356 | 1.062 | 49.163 | 6.756 | 1.00 | 50.00 | O |
| ATOM | 8731 | OE2 | GLU | F | 356 | −0.891 | 49.956 | 7.343 | 1.00 | 50.00 | O |
| ATOM | 8732 | N | THR | F | 357 | −0.950 | 47.915 | 1.427 | 1.00 | 50.00 | N |
| ATOM | 8733 | CA | THR | F | 357 | −1.678 | 47.042 | 0.505 | 1.00 | 50.00 | C |
| ATOM | 8734 | C | THR | F | 357 | −0.867 | 45.862 | −0.026 | 1.00 | 50.00 | C |
| ATOM | 8735 | O | THR | F | 357 | −1.098 | 45.441 | −1.159 | 1.00 | 50.00 | O |
| ATOM | 8736 | CB | THR | F | 357 | −2.273 | 47.986 | −0.543 | 1.00 | 50.00 | C |
| ATOM | 8737 | OG1 | THR | F | 357 | −3.046 | 49.019 | 0.052 | 1.00 | 50.00 | O |
| ATOM | 8738 | CG2 | THR | F | 357 | −3.241 | 47.312 | −1.512 | 1.00 | 50.00 | C |
| ATOM | 8739 | N | CYS | F | 358 | 0.042 | 45.249 | 0.718 | 1.00 | 50.00 | N |
| ATOM | 8740 | CA | CYS | F | 358 | 0.888 | 44.170 | 0.209 | 1.00 | 50.00 | C |
| ATOM | 8741 | C | CYS | F | 358 | 1.270 | 43.147 | 1.273 | 1.00 | 50.00 | C |
| ATOM | 8742 | O | CYS | F | 358 | 1.814 | 43.501 | 2.318 | 1.00 | 50.00 | O |
| ATOM | 8743 | CB | CYS | F | 358 | 2.133 | 44.739 | −0.473 | 1.00 | 50.00 | C |
| ATOM | 8744 | SG | CYS | F | 358 | 1.848 | 46.141 | −1.568 | 1.00 | 50.00 | S |
| ATOM | 8745 | N | LYS | F | 359 | 1.081 | 41.871 | 0.973 | 1.00 | 50.00 | N |
| ATOM | 8746 | CA | LYS | F | 359 | 1.806 | 40.751 | 1.580 | 1.00 | 50.00 | C |
| ATOM | 8747 | C | LYS | F | 359 | 3.201 | 40.711 | 0.967 | 1.00 | 50.00 | C |
| ATOM | 8748 | O | LYS | F | 359 | 3.367 | 40.732 | −0.252 | 1.00 | 50.00 | O |
| ATOM | 8749 | CB | LYS | F | 359 | 1.182 | 39.359 | 1.448 | 1.00 | 50.00 | C |
| ATOM | 8750 | CG | LYS | F | 359 | −0.300 | 39.183 | 1.779 | 1.00 | 50.00 | C |
| ATOM | 8751 | CD | LYS | F | 359 | −0.975 | 37.946 | 1.183 | 1.00 | 50.00 | C |
| ATOM | 8752 | CE | LYS | F | 359 | −2.500 | 37.988 | 1.266 | 1.00 | 50.00 | C |
| ATOM | 8753 | NZ | LYS | F | 359 | −3.143 | 36.839 | 0.612 | 1.00 | 50.00 | N |
| ATOM | 8754 | N | VAL | F | 360 | 4.204 | 40.692 | 1.828 | 1.00 | 50.00 | N |
| ATOM | 8755 | CA | VAL | F | 360 | 5.599 | 40.925 | 1.457 | 1.00 | 50.00 | C |
| ATOM | 8756 | C | VAL | F | 360 | 6.337 | 39.624 | 1.752 | 1.00 | 50.00 | C |
| ATOM | 8757 | O | VAL | F | 360 | 6.517 | 39.217 | 2.899 | 1.00 | 50.00 | O |
| ATOM | 8758 | CB | VAL | F | 360 | 6.198 | 42.144 | 2.162 | 1.00 | 50.00 | C |
| ATOM | 8759 | CG1 | VAL | F | 360 | 7.706 | 42.296 | 1.952 | 1.00 | 50.00 | C |
| ATOM | 8760 | CG2 | VAL | F | 360 | 5.470 | 43.441 | 1.810 | 1.00 | 50.00 | C |
| ATOM | 8761 | N | GLN | F | 361 | 6.860 | 39.052 | 0.682 | 1.00 | 50.00 | N |
| ATOM | 8762 | CA | GLN | F | 361 | 7.929 | 38.056 | 0.728 | 1.00 | 50.00 | C |
| ATOM | 8763 | C | GLN | F | 361 | 9.346 | 38.611 | 0.781 | 1.00 | 50.00 | C |
| ATOM | 8764 | O | GLN | F | 361 | 9.605 | 39.792 | 0.555 | 1.00 | 50.00 | O |
| ATOM | 8765 | CB | GLN | F | 361 | 7.734 | 37.144 | −0.483 | 1.00 | 50.00 | C |
| ATOM | 8766 | CG | GLN | F | 361 | 6.568 | 36.170 | −0.365 | 1.00 | 50.00 | C |
| ATOM | 8767 | CD | GLN | F | 361 | 6.631 | 35.360 | 0.920 | 1.00 | 50.00 | C |
| ATOM | 8768 | OE1 | GLN | F | 361 | 7.632 | 34.698 | 1.190 | 1.00 | 50.00 | O |
| ATOM | 8769 | NE2 | GLN | F | 361 | 5.674 | 35.578 | 1.802 | 1.00 | 50.00 | N |
| ATOM | 8770 | N | SER | F | 362 | 10.264 | 37.692 | 1.032 | 1.00 | 50.00 | N |
| ATOM | 8771 | CA | SER | F | 362 | 11.703 | 37.891 | 0.860 | 1.00 | 50.00 | C |
| ATOM | 8772 | C | SER | F | 362 | 12.091 | 38.418 | −0.518 | 1.00 | 50.00 | C |
| ATOM | 8773 | O | SER | F | 362 | 12.587 | 39.540 | −0.617 | 1.00 | 50.00 | O |
| ATOM | 8774 | CB | SER | F | 362 | 12.430 | 36.595 | 1.220 | 1.00 | 50.00 | C |
| ATOM | 8775 | OG | SER | F | 362 | 12.177 | 36.186 | 2.556 | 1.00 | 50.00 | O |
| ATOM | 8776 | N | ASN | F | 363 | 11.788 | 37.661 | −1.560 | 1.00 | 50.00 | N |
| ATOM | 8777 | CA | ASN | F | 363 | 11.875 | 38.079 | −2.960 | 1.00 | 50.00 | C |
| ATOM | 8778 | C | ASN | F | 363 | 10.529 | 38.233 | −3.665 | 1.00 | 50.00 | C |
| ATOM | 8779 | O | ASN | F | 363 | 10.587 | 38.300 | −4.892 | 1.00 | 50.00 | O |
| ATOM | 8780 | CB | ASN | F | 363 | 12.765 | 37.145 | −3.783 | 1.00 | 50.00 | C |
| ATOM | 8781 | CG | ASN | F | 363 | 14.018 | 36.526 | −3.184 | 1.00 | 50.00 | C |
| ATOM | 8782 | OD1 | ASN | F | 363 | 13.921 | 35.572 | −2.414 | 1.00 | 50.00 | O |
| ATOM | 8783 | ND2 | ASN | F | 363 | 15.183 | 37.043 | −3.527 | 1.00 | 50.00 | N |
| ATOM | 8784 | N | ARG | F | 364 | 9.354 | 38.307 | −3.054 | 1.00 | 50.00 | N |
| ATOM | 8785 | CA | ARG | F | 364 | 8.134 | 38.894 | −3.616 | 1.00 | 50.00 | C |
| ATOM | 8786 | C | ARG | F | 364 | 7.477 | 39.989 | −2.778 | 1.00 | 50.00 | C |
| ATOM | 8787 | O | ARG | F | 364 | 7.844 | 40.237 | −1.630 | 1.00 | 50.00 | O |
| ATOM | 8788 | CB | ARG | F | 364 | 7.220 | 38.070 | −4.524 | 1.00 | 50.00 | C |
| ATOM | 8789 | CG | ARG | F | 364 | 7.926 | 37.441 | −5.723 | 1.00 | 50.00 | C |
| ATOM | 8790 | CD | ARG | F | 364 | 7.003 | 36.893 | −6.808 | 1.00 | 50.00 | C |
| ATOM | 8791 | NE | ARG | F | 364 | 7.699 | 36.088 | −7.808 | 1.00 | 50.00 | N |
| ATOM | 8792 | CZ | ARG | F | 364 | 7.110 | 35.181 | −8.567 | 1.00 | 50.00 | C |
| ATOM | 8793 | NH1 | ARG | F | 364 | 5.840 | 35.179 | −8.917 | 1.00 | 50.00 | N |
| ATOM | 8794 | NH2 | ARG | F | 364 | 7.835 | 34.184 | −9.027 | 1.00 | 50.00 | N |
| ATOM | 8795 | N | VAL | F | 365 | 6.586 | 40.772 | −3.365 | 1.00 | 50.00 | N |
| ATOM | 8796 | CA | VAL | F | 365 | 5.674 | 41.725 | −2.730 | 1.00 | 50.00 | C |
| ATOM | 8797 | C | VAL | F | 365 | 4.499 | 41.580 | −3.688 | 1.00 | 50.00 | C |
| ATOM | 8798 | O | VAL | F | 365 | 4.434 | 42.186 | −4.759 | 1.00 | 50.00 | O |
| ATOM | 8799 | CB | VAL | F | 365 | 6.059 | 43.195 | −2.537 | 1.00 | 50.00 | C |
| ATOM | 8800 | CG1 | VAL | F | 365 | 4.964 | 44.200 | −2.190 | 1.00 | 50.00 | C |
| ATOM | 8801 | CG2 | VAL | F | 365 | 7.177 | 43.470 | −1.533 | 1.00 | 50.00 | C |
| ATOM | 8802 | N | PHE | F | 366 | 3.532 | 40.808 | −3.222 | 1.00 | 50.00 | N |
| ATOM | 8803 | CA | PHE | F | 366 | 2.175 | 40.783 | −3.763 | 1.00 | 50.00 | C |
| ATOM | 8804 | C | PHE | F | 366 | 1.500 | 42.102 | −3.409 | 1.00 | 50.00 | C |
| ATOM | 8805 | O | PHE | F | 366 | 1.368 | 42.398 | −2.224 | 1.00 | 50.00 | O |
| ATOM | 8806 | CB | PHE | F | 366 | 1.537 | 39.473 | −3.289 | 1.00 | 50.00 | C |
| ATOM | 8807 | CG | PHE | F | 366 | 0.295 | 39.189 | −2.471 | 1.00 | 50.00 | C |
| ATOM | 8808 | CD1 | PHE | F | 366 | −0.008 | 40.103 | −1.531 | 1.00 | 50.00 | C |
| ATOM | 8809 | CD2 | PHE | F | 366 | −0.675 | 38.328 | −2.844 | 1.00 | 50.00 | C |
| ATOM | 8810 | CE1 | PHE | F | 366 | −1.290 | 40.395 | −1.236 | 1.00 | 50.00 | C |
| ATOM | 8811 | CE2 | PHE | F | 366 | −1.964 | 38.568 | −2.529 | 1.00 | 50.00 | C |
| ATOM | 8812 | CZ | PHE | F | 366 | −2.271 | 39.632 | −1.759 | 1.00 | 50.00 | C |
| ATOM | 8813 | N | CYS | F | 367 | 1.126 | 42.955 | −4.346 | 1.00 | 50.00 | N |
| ATOM | 8814 | CA | CYS | F | 367 | 0.252 | 44.089 | −4.048 | 1.00 | 50.00 | C |
| ATOM | 8815 | C | CYS | F | 367 | −1.130 | 43.863 | −4.644 | 1.00 | 50.00 | C |
| ATOM | 8816 | O | CYS | F | 367 | −1.316 | 43.324 | −5.733 | 1.00 | 50.00 | O |
| ATOM | 8817 | CB | CYS | F | 367 | 0.874 | 45.419 | −4.465 | 1.00 | 50.00 | C |
| ATOM | 8818 | SG | CYS | F | 367 | 2.336 | 45.751 | −3.470 | 1.00 | 50.00 | S |
| ATOM | 8819 | N | ASP | F | 368 | −2.131 | 44.322 | −3.913 | 1.00 | 50.00 | N |
| ATOM | 8820 | CA | ASP | F | 368 | −3.466 | 44.600 | −4.444 | 1.00 | 50.00 | C |
| ATOM | 8821 | C | ASP | F | 368 | −3.591 | 45.918 | −5.206 | 1.00 | 50.00 | C |
| ATOM | 8822 | O | ASP | F | 368 | −4.398 | 46.007 | −6.131 | 1.00 | 50.00 | O |
| ATOM | 8823 | CB | ASP | F | 368 | −4.409 | 44.445 | −3.249 | 1.00 | 50.00 | C |
| ATOM | 8824 | CG | ASP | F | 368 | −4.552 | 43.063 | −2.623 | 1.00 | 50.00 | C |
| ATOM | 8825 | OD1 | ASP | F | 368 | −4.839 | 42.078 | −3.337 | 1.00 | 50.00 | O |
| ATOM | 8826 | OD2 | ASP | F | 368 | −4.347 | 42.944 | −1.396 | 1.00 | 50.00 | O |
| ATOM | 8827 | N | THR | F | 369 | −2.817 | 46.934 | −4.858 | 1.00 | 50.00 | N |
| ATOM | 8828 | CA | THR | F | 369 | −2.805 | 48.290 | −5.408 | 1.00 | 50.00 | C |
| ATOM | 8829 | C | THR | F | 369 | −1.393 | 48.840 | −5.232 | 1.00 | 50.00 | C |
| ATOM | 8830 | O | THR | F | 369 | −0.616 | 48.393 | −4.388 | 1.00 | 50.00 | O |
| ATOM | 8831 | CB | THR | F | 369 | −3.810 | 49.168 | −4.659 | 1.00 | 50.00 | C |
| ATOM | 8832 | OG1 | THR | F | 369 | −5.103 | 48.581 | −4.674 | 1.00 | 50.00 | O |
| ATOM | 8833 | CG2 | THR | F | 369 | −3.973 | 50.617 | −5.112 | 1.00 | 50.00 | C |
| ATOM | 8834 | N | MET | F | 370 | −1.047 | 49.886 | −5.964 | 1.00 | 50.00 | N |
| ATOM | 8835 | CA | MET | F | 370 | 0.242 | 50.567 | −5.827 | 1.00 | 50.00 | C |
| ATOM | 8836 | C | MET | F | 370 | 0.087 | 51.971 | −5.263 | 1.00 | 50.00 | C |
| ATOM | 8837 | O | MET | F | 370 | 0.114 | 52.994 | −5.946 | 1.00 | 50.00 | O |
| ATOM | 8838 | CB | MET | F | 370 | 1.007 | 50.511 | −7.147 | 1.00 | 50.00 | C |
| ATOM | 8839 | CG | MET | F | 370 | 1.513 | 49.093 | −7.391 | 1.00 | 50.00 | C |
| ATOM | 8840 | SD | MET | F | 370 | 2.636 | 48.508 | −6.111 | 1.00 | 50.00 | S |
| ATOM | 8841 | CE | MET | F | 370 | 4.042 | 49.575 | −6.462 | 1.00 | 50.00 | C |
| ATOM | 8842 | N | ASN | F | 371 | −0.027 | 51.967 | −3.946 | 1.00 | 50.00 | N |
| ATOM | 8843 | CA | ASN | F | 371 | −0.281 | 53.160 | −3.143 | 1.00 | 50.00 | C |
| ATOM | 8844 | C | ASN | F | 371 | 1.040 | 53.882 | −2.873 | 1.00 | 50.00 | C |
| ATOM | 8845 | O | ASN | F | 371 | 1.843 | 53.257 | −2.184 | 1.00 | 50.00 | O |
| ATOM | 8846 | CB | ASN | F | 371 | −1.206 | 52.638 | −2.036 | 1.00 | 50.00 | C |
| ATOM | 8847 | CG | ASN | F | 371 | −2.677 | 52.398 | −2.365 | 1.00 | 50.00 | C |
| ATOM | 8848 | OD1 | ASN | F | 371 | −3.188 | 52.820 | −3.402 | 1.00 | 50.00 | O |
| ATOM | 8849 | ND2 | ASN | F | 371 | −3.491 | 51.809 | −1.509 | 1.00 | 50.00 | N |
| ATOM | 8850 | N | SER | F | 372 | 1.393 | 55.034 | −3.428 | 1.00 | 50.00 | N |
| ATOM | 8851 | CA | SER | F | 372 | 2.632 | 55.798 | −3.233 | 1.00 | 50.00 | C |
| ATOM | 8852 | C | SER | F | 372 | 2.626 | 57.328 | −3.126 | 1.00 | 50.00 | C |
| ATOM | 8853 | O | SER | F | 372 | 1.630 | 57.970 | −3.452 | 1.00 | 50.00 | O |
| ATOM | 8854 | CB | SER | F | 372 | 3.567 | 55.353 | −4.358 | 1.00 | 50.00 | C |
| ATOM | 8855 | OG | SER | F | 372 | 4.880 | 55.882 | −4.242 | 1.00 | 50.00 | O |
| ATOM | 8856 | N | LEU | F | 373 | 3.635 | 58.051 | −2.655 | 1.00 | 50.00 | N |
| ATOM | 8857 | CA | LEU | F | 373 | 3.615 | 59.485 | −2.338 | 1.00 | 50.00 | C |
| ATOM | 8858 | C | LEU | F | 373 | 4.777 | 60.270 | −2.945 | 1.00 | 50.00 | C |
| ATOM | 8859 | O | LEU | F | 373 | 5.860 | 59.705 | −3.093 | 1.00 | 50.00 | O |
| ATOM | 8860 | CB | LEU | F | 373 | 3.592 | 59.734 | −0.826 | 1.00 | 50.00 | C |
| ATOM | 8861 | CG | LEU | F | 373 | 2.297 | 59.582 | −0.027 | 1.00 | 50.00 | C |
| ATOM | 8862 | CD1 | LEU | F | 373 | 1.890 | 58.122 | 0.145 | 1.00 | 50.00 | C |
| ATOM | 8863 | CD2 | LEU | F | 373 | 2.361 | 60.106 | 1.408 | 1.00 | 50.00 | C |
| ATOM | 8864 | N | THR | F | 374 | 4.648 | 61.560 | −3.227 | 1.00 | 50.00 | N |
| ATOM | 8865 | CA | THR | F | 374 | 5.749 | 62.469 | −3.565 | 1.00 | 50.00 | C |
| ATOM | 8866 | C | THR | F | 374 | 6.560 | 62.934 | −2.360 | 1.00 | 50.00 | C |
| ATOM | 8867 | O | THR | F | 374 | 6.015 | 63.227 | −1.296 | 1.00 | 50.00 | O |
| ATOM | 8868 | CB | THR | F | 374 | 5.362 | 63.644 | −4.468 | 1.00 | 50.00 | C |
| ATOM | 8869 | OG1 | THR | F | 374 | 4.708 | 63.162 | −5.633 | 1.00 | 50.00 | O |
| ATOM | 8870 | CG2 | THR | F | 374 | 6.501 | 64.541 | −4.955 | 1.00 | 50.00 | C |
| ATOM | 8871 | N | LEU | F | 375 | 7.871 | 62.980 | −2.540 | 1.00 | 50.00 | N |
| ATOM | 8872 | CA | LEU | F | 375 | 8.855 | 63.255 | −1.492 | 1.00 | 50.00 | C |
| ATOM | 8873 | C | LEU | F | 375 | 9.493 | 64.617 | −1.745 | 1.00 | 50.00 | C |
| ATOM | 8874 | O | LEU | F | 375 | 9.620 | 64.994 | −2.910 | 1.00 | 50.00 | O |
| ATOM | 8875 | CB | LEU | F | 375 | 9.922 | 62.163 | −1.373 | 1.00 | 50.00 | C |
| ATOM | 8876 | CG | LEU | F | 375 | 9.485 | 60.843 | −0.737 | 1.00 | 50.00 | C |
| ATOM | 8877 | CD1 | LEU | F | 375 | 8.397 | 60.191 | −1.583 | 1.00 | 50.00 | C |
| ATOM | 8878 | CD2 | LEU | F | 375 | 10.557 | 59.769 | −0.555 | 1.00 | 50.00 | C |
| ATOM | 8879 | N | PRO | F | 376 | 9.977 | 65.354 | −0.753 | 1.00 | 50.00 | N |
| ATOM | 8880 | CA | PRO | F | 376 | 10.845 | 66.518 | −0.942 | 1.00 | 50.00 | C |
| ATOM | 8881 | C | PRO | F | 376 | 11.912 | 66.395 | −2.023 | 1.00 | 50.00 | C |
| ATOM | 8882 | O | PRO | F | 376 | 12.507 | 65.326 | −2.161 | 1.00 | 50.00 | O |
| ATOM | 8883 | CB | PRO | F | 376 | 11.493 | 66.700 | 0.429 | 1.00 | 50.00 | C |
| ATOM | 8884 | CG | PRO | F | 376 | 10.519 | 66.111 | 1.446 | 1.00 | 50.00 | C |
| ATOM | 8885 | CD | PRO | F | 376 | 9.743 | 65.059 | 0.660 | 1.00 | 50.00 | C |
| ATOM | 8886 | N | SER | F | 377 | 12.146 | 67.444 | −2.794 | 1.00 | 50.00 | N |
| ATOM | 8887 | CA | SER | F | 377 | 13.227 | 67.488 | −3.782 | 1.00 | 50.00 | C |
| ATOM | 8888 | C | SER | F | 377 | 14.581 | 67.021 | −3.260 | 1.00 | 50.00 | C |
| ATOM | 8889 | O | SER | F | 377 | 15.140 | 66.075 | −3.812 | 1.00 | 50.00 | O |
| ATOM | 8890 | CB | SER | F | 377 | 13.317 | 68.869 | −4.430 | 1.00 | 50.00 | C |
| ATOM | 8891 | OG | SER | F | 377 | 12.134 | 69.180 | −5.151 | 1.00 | 50.00 | O |
| ATOM | 8892 | N | GLU | F | 378 | 15.017 | 67.554 | −2.130 | 1.00 | 50.00 | N |
| ATOM | 8893 | CA | GLU | F | 378 | 16.156 | 67.088 | −1.339 | 1.00 | 50.00 | C |
| ATOM | 8894 | C | GLU | F | 378 | 16.257 | 65.583 | −1.123 | 1.00 | 50.00 | C |
| ATOM | 8895 | O | GLU | F | 378 | 17.244 | 65.041 | −1.615 | 1.00 | 50.00 | O |
| ATOM | 8896 | CB | GLU | F | 378 | 16.212 | 67.887 | −0.036 | 1.00 | 50.00 | C |
| ATOM | 8897 | CG | GLU | F | 378 | 16.359 | 69.398 | −0.214 | 1.00 | 50.00 | C |
| ATOM | 8898 | CD | GLU | F | 378 | 17.768 | 69.844 | −0.572 | 1.00 | 50.00 | C |
| ATOM | 8899 | OE1 | GLU | F | 378 | 18.068 | 69.980 | −1.778 | 1.00 | 50.00 | O |
| ATOM | 8900 | OE2 | GLU | F | 378 | 18.593 | 70.048 | 0.346 | 1.00 | 50.00 | O |
| ATOM | 8901 | N | VAL | F | 379 | 15.283 | 64.883 | −0.563 | 1.00 | 50.00 | N |
| ATOM | 8902 | CA | VAL | F | 379 | 15.199 | 63.420 | −0.557 | 1.00 | 50.00 | C |
| ATOM | 8903 | C | VAL | F | 379 | 15.509 | 62.730 | −1.883 | 1.00 | 50.00 | C |
| ATOM | 8904 | O | VAL | F | 379 | 16.399 | 61.881 | −1.914 | 1.00 | 50.00 | O |
| ATOM | 8905 | CB | VAL | F | 379 | 13.859 | 62.975 | 0.036 | 1.00 | 50.00 | C |
| ATOM | 8906 | CG1 | VAL | F | 379 | 13.572 | 61.478 | −0.072 | 1.00 | 50.00 | C |
| ATOM | 8907 | CG2 | VAL | F | 379 | 13.629 | 63.349 | 1.500 | 1.00 | 50.00 | C |
| ATOM | 8908 | N | ASN | F | 380 | 14.797 | 63.054 | −2.952 | 1.00 | 50.00 | N |
| ATOM | 8909 | CA | ASN | F | 380 | 15.043 | 62.542 | −4.301 | 1.00 | 50.00 | C |
| ATOM | 8910 | C | ASN | F | 380 | 16.456 | 62.745 | −4.833 | 1.00 | 50.00 | C |
| ATOM | 8911 | O | ASN | F | 380 | 17.114 | 61.759 | −5.162 | 1.00 | 50.00 | O |
| ATOM | 8912 | CB | ASN | F | 380 | 14.042 | 63.119 | −5.304 | 1.00 | 50.00 | C |
| ATOM | 8913 | CG | ASN | F | 380 | 12.582 | 62.707 | −5.187 | 1.00 | 50.00 | C |
| ATOM | 8914 | OD1 | ASN | F | 380 | 11.672 | 63.535 | −5.222 | 1.00 | 50.00 | O |
| ATOM | 8915 | ND2 | ASN | F | 380 | 12.324 | 61.417 | −5.085 | 1.00 | 50.00 | N |
| ATOM | 8916 | N | LEU | F | 381 | 16.939 | 63.976 | −4.852 | 1.00 | 50.00 | N |
| ATOM | 8917 | CA | LEU | F | 381 | 18.346 | 64.351 | −4.992 | 1.00 | 50.00 | C |
| ATOM | 8918 | C | LEU | F | 381 | 19.324 | 63.429 | −4.273 | 1.00 | 50.00 | C |
| ATOM | 8919 | O | LEU | F | 381 | 20.067 | 62.700 | −4.927 | 1.00 | 50.00 | O |
| ATOM | 8920 | CB | LEU | F | 381 | 18.417 | 65.819 | −4.564 | 1.00 | 50.00 | C |
| ATOM | 8921 | CG | LEU | F | 381 | 18.269 | 66.916 | −5.618 | 1.00 | 50.00 | C |
| ATOM | 8922 | CD1 | LEU | F | 381 | 16.905 | 66.937 | −6.306 | 1.00 | 50.00 | C |
| ATOM | 8923 | CD2 | LEU | F | 381 | 18.530 | 68.283 | −4.988 | 1.00 | 50.00 | C |
| ATOM | 8924 | N | CYS | F | 382 | 19.236 | 63.369 | −2.955 | 1.00 | 50.00 | N |
| ATOM | 8925 | CA | CYS | F | 382 | 19.844 | 62.357 | −2.092 | 1.00 | 50.00 | C |
| ATOM | 8926 | C | CYS | F | 382 | 19.817 | 60.885 | −2.496 | 1.00 | 50.00 | C |
| ATOM | 8927 | O | CYS | F | 382 | 20.777 | 60.179 | −2.192 | 1.00 | 50.00 | O |
| ATOM | 8928 | CB | CYS | F | 382 | 19.198 | 62.653 | −0.738 | 1.00 | 50.00 | C |
| ATOM | 8929 | SG | CYS | F | 382 | 20.449 | 63.067 | 0.483 | 1.00 | 50.00 | S |
| ATOM | 8930 | N | ASN | F | 383 | 18.800 | 60.388 | −3.181 | 1.00 | 50.00 | N |
| ATOM | 8931 | CA | ASN | F | 383 | 18.784 | 59.070 | −3.822 | 1.00 | 50.00 | C |
| ATOM | 8932 | C | ASN | F | 383 | 18.744 | 59.109 | −5.348 | 1.00 | 50.00 | C |
| ATOM | 8933 | O | ASN | F | 383 | 17.945 | 58.423 | −5.984 | 1.00 | 50.00 | O |
| ATOM | 8934 | CB | ASN | F | 383 | 17.612 | 58.259 | −3.265 | 1.00 | 50.00 | C |
| ATOM | 8935 | CG | ASN | F | 383 | 17.746 | 57.589 | −1.906 | 1.00 | 50.00 | C |
| ATOM | 8936 | OD1 | ASN | F | 383 | 16.945 | 57.826 | −1.004 | 1.00 | 50.00 | O |
| ATOM | 8937 | ND2 | ASN | F | 383 | 18.767 | 56.773 | −1.716 | 1.00 | 50.00 | N |
| ATOM | 8938 | N | VAL | F | 384 | 19.629 | 59.857 | −5.985 | 1.00 | 50.00 | N |
| ATOM | 8939 | CA | VAL | F | 384 | 19.792 | 59.899 | −7.440 | 1.00 | 50.00 | C |
| ATOM | 8940 | C | VAL | F | 384 | 21.299 | 60.042 | −7.612 | 1.00 | 50.00 | C |
| ATOM | 8941 | O | VAL | F | 384 | 21.884 | 61.034 | −7.176 | 1.00 | 50.00 | O |
| ATOM | 8942 | CB | VAL | F | 384 | 18.974 | 60.994 | −8.131 | 1.00 | 50.00 | C |
| ATOM | 8943 | CG1 | VAL | F | 384 | 19.351 | 61.251 | −9.591 | 1.00 | 50.00 | C |
| ATOM | 8944 | CG2 | VAL | F | 384 | 17.468 | 60.731 | −8.138 | 1.00 | 50.00 | C |
| ATOM | 8945 | N | ASP | F | 385 | 21.925 | 59.079 | −8.271 | 1.00 | 50.00 | N |
| ATOM | 8946 | CA | ASP | F | 385 | 23.362 | 59.066 | −8.548 | 1.00 | 50.00 | C |
| ATOM | 8947 | C | ASP | F | 385 | 24.188 | 58.936 | −7.271 | 1.00 | 50.00 | C |
| ATOM | 8948 | O | ASP | F | 385 | 23.772 | 58.331 | −6.283 | 1.00 | 50.00 | O |
| ATOM | 8949 | CB | ASP | F | 385 | 23.747 | 60.168 | −9.541 | 1.00 | 50.00 | C |
| ATOM | 8950 | CG | ASP | F | 385 | 23.244 | 60.065 | −10.972 | 1.00 | 50.00 | C |
| ATOM | 8951 | OD1 | ASP | F | 385 | 23.600 | 59.089 | −11.668 | 1.00 | 50.00 | O |
| ATOM | 8952 | OD2 | ASP | F | 385 | 22.521 | 60.979 | −11.425 | 1.00 | 50.00 | O |
| ATOM | 8953 | N | ILE | F | 386 | 25.370 | 59.529 | −7.281 | 1.00 | 50.00 | N |
| ATOM | 8954 | CA | ILE | F | 386 | 26.179 | 59.860 | −6.108 | 1.00 | 50.00 | C |
| ATOM | 8955 | C | ILE | F | 386 | 25.923 | 61.344 | −5.845 | 1.00 | 50.00 | C |
| ATOM | 8956 | O | ILE | F | 386 | 26.840 | 62.150 | −5.999 | 1.00 | 50.00 | O |
| ATOM | 8957 | CB | ILE | F | 386 | 27.603 | 59.405 | −6.449 | 1.00 | 50.00 | C |
| ATOM | 8958 | CG1 | ILE | F | 386 | 27.785 | 57.908 | −6.716 | 1.00 | 50.00 | C |
| ATOM | 8959 | CG2 | ILE | F | 386 | 28.674 | 59.793 | −5.430 | 1.00 | 50.00 | C |
| ATOM | 8960 | CD1 | ILE | F | 386 | 29.148 | 57.448 | −7.230 | 1.00 | 50.00 | C |
| ATOM | 8961 | N | PHE | F | 387 | 24.733 | 61.760 | −5.433 | 1.00 | 50.00 | N |
| ATOM | 8962 | CA | PHE | F | 387 | 24.430 | 63.130 | −5.007 | 1.00 | 50.00 | C |
| ATOM | 8963 | C | PHE | F | 387 | 24.431 | 63.367 | −3.500 | 1.00 | 50.00 | C |
| ATOM | 8964 | O | PHE | F | 387 | 24.974 | 64.388 | −3.079 | 1.00 | 50.00 | O |
| ATOM | 8965 | CB | PHE | F | 387 | 23.144 | 63.656 | −5.646 | 1.00 | 50.00 | C |
| ATOM | 8966 | CG | PHE | F | 387 | 22.842 | 65.120 | −5.397 | 1.00 | 50.00 | C |
| ATOM | 8967 | CD1 | PHE | F | 387 | 23.476 | 66.095 | −6.082 | 1.00 | 50.00 | C |
| ATOM | 8968 | CD2 | PHE | F | 387 | 22.024 | 65.457 | −4.379 | 1.00 | 50.00 | C |
| ATOM | 8969 | CE1 | PHE | F | 387 | 23.295 | 67.391 | −5.755 | 1.00 | 50.00 | C |
| ATOM | 8970 | CE2 | PHE | F | 387 | 21.800 | 66.754 | −4.089 | 1.00 | 50.00 | C |
| ATOM | 8971 | CZ | PHE | F | 387 | 22.452 | 67.724 | −4.757 | 1.00 | 50.00 | C |
| ATOM | 8972 | N | ASN | F | 388 | 23.860 | 62.506 | −2.673 | 1.00 | 50.00 | N |
| ATOM | 8973 | CA | ASN | F | 388 | 24.135 | 62.429 | −1.236 | 1.00 | 50.00 | C |
| ATOM | 8974 | C | ASN | F | 388 | 25.547 | 62.675 | −0.705 | 1.00 | 50.00 | C |
| ATOM | 8975 | O | ASN | F | 388 | 25.635 | 63.324 | 0.337 | 1.00 | 50.00 | O |
| ATOM | 8976 | CB | ASN | F | 388 | 23.510 | 61.144 | −0.691 | 1.00 | 50.00 | C |
| ATOM | 8977 | CG | ASN | F | 388 | 24.043 | 59.799 | −1.168 | 1.00 | 50.00 | C |
| ATOM | 8978 | OD1 | ASN | F | 388 | 24.987 | 59.278 | −0.578 | 1.00 | 50.00 | O |
| ATOM | 8979 | ND2 | ASN | F | 388 | 23.597 | 59.252 | −2.285 | 1.00 | 50.00 | N |
| ATOM | 8980 | N | PRO | F | 389 | 26.651 | 62.263 | −1.313 | 1.00 | 50.00 | N |
| ATOM | 8981 | CA | PRO | F | 389 | 28.029 | 62.577 | −0.922 | 1.00 | 50.00 | C |
| ATOM | 8982 | C | PRO | F | 389 | 28.569 | 63.982 | −1.140 | 1.00 | 50.00 | C |
| ATOM | 8983 | O | PRO | F | 389 | 29.373 | 64.482 | −0.354 | 1.00 | 50.00 | O |
| ATOM | 8984 | CB | PRO | F | 389 | 28.935 | 61.736 | −1.821 | 1.00 | 50.00 | C |
| ATOM | 8985 | CG | PRO | F | 389 | 27.935 | 60.634 | −2.138 | 1.00 | 50.00 | C |
| ATOM | 8986 | CD | PRO | F | 389 | 26.660 | 61.387 | −2.482 | 1.00 | 50.00 | C |
| ATOM | 8987 | N | LYS | F | 390 | 28.128 | 64.566 | −2.241 | 1.00 | 50.00 | N |
| ATOM | 8988 | CA | LYS | F | 390 | 28.303 | 65.983 | −2.550 | 1.00 | 50.00 | C |
| ATOM | 8989 | C | LYS | F | 390 | 27.655 | 66.967 | −1.583 | 1.00 | 50.00 | C |
| ATOM | 8990 | O | LYS | F | 390 | 27.815 | 68.155 | −1.859 | 1.00 | 50.00 | O |
| ATOM | 8991 | CB | LYS | F | 390 | 27.785 | 66.179 | −3.975 | 1.00 | 50.00 | C |
| ATOM | 8992 | CG | LYS | F | 390 | 28.557 | 65.427 | −5.059 | 1.00 | 50.00 | C |
| ATOM | 8993 | CD | LYS | F | 390 | 28.110 | 65.842 | −6.459 | 1.00 | 50.00 | C |
| ATOM | 8994 | CE | LYS | F | 390 | 28.876 | 65.084 | −7.540 | 1.00 | 50.00 | C |
| ATOM | 8995 | NZ | LYS | F | 390 | 28.476 | 65.580 | −8.865 | 1.00 | 50.00 | N |
| ATOM | 8996 | N | TYR | F | 391 | 27.020 | 66.520 | −0.508 | 1.00 | 50.00 | N |
| ATOM | 8997 | CA | TYR | F | 391 | 26.535 | 67.270 | 0.651 | 1.00 | 50.00 | C |
| ATOM | 8998 | C | TYR | F | 391 | 25.081 | 67.005 | 1.024 | 1.00 | 50.00 | C |
| ATOM | 8999 | O | TYR | F | 391 | 24.740 | 67.315 | 2.166 | 1.00 | 50.00 | O |
| ATOM | 9000 | CB | TYR | F | 391 | 26.955 | 68.741 | 0.705 | 1.00 | 50.00 | C |
| ATOM | 9001 | CG | TYR | F | 391 | 26.474 | 69.627 | 1.831 | 1.00 | 50.00 | C |
| ATOM | 9002 | CD1 | TYR | F | 391 | 27.026 | 69.564 | 3.060 | 1.00 | 50.00 | C |
| ATOM | 9003 | CD2 | TYR | F | 391 | 25.406 | 70.422 | 1.610 | 1.00 | 50.00 | C |
| ATOM | 9004 | CE1 | TYR | F | 391 | 26.516 | 70.304 | 4.064 | 1.00 | 50.00 | C |
| ATOM | 9005 | CE2 | TYR | F | 391 | 24.896 | 71.162 | 2.614 | 1.00 | 50.00 | C |
| ATOM | 9006 | CZ | TYR | F | 391 | 25.454 | 71.105 | 3.840 | 1.00 | 50.00 | C |
| ATOM | 9007 | OH | TYR | F | 391 | 24.963 | 71.886 | 4.852 | 1.00 | 50.00 | O |
| ATOM | 9008 | N | ASP | F | 392 | 24.200 | 66.476 | 0.189 | 1.00 | 50.00 | N |
| ATOM | 9009 | CA | ASP | F | 392 | 22.764 | 66.467 | 0.469 | 1.00 | 50.00 | C |
| ATOM | 9010 | C | ASP | F | 392 | 22.375 | 65.545 | 1.620 | 1.00 | 50.00 | C |
| ATOM | 9011 | O | ASP | F | 392 | 23.194 | 64.976 | 2.342 | 1.00 | 50.00 | O |
| ATOM | 9012 | CB | ASP | F | 392 | 22.003 | 66.300 | −0.847 | 1.00 | 50.00 | C |
| ATOM | 9013 | CG | ASP | F | 392 | 20.586 | 66.856 | −0.856 | 1.00 | 50.00 | C |
| ATOM | 9014 | OD1 | ASP | F | 392 | 20.423 | 68.094 | −0.925 | 1.00 | 50.00 | O |
| ATOM | 9015 | OD2 | ASP | F | 392 | 19.621 | 66.064 | −0.804 | 1.00 | 50.00 | O |
| ATOM | 9016 | N | CYS | F | 393 | 21.075 | 65.521 | 1.848 | 1.00 | 50.00 | N |
| ATOM | 9017 | CA | CYS | F | 393 | 20.411 | 64.919 | 3.001 | 1.00 | 50.00 | C |
| ATOM | 9018 | C | CYS | F | 393 | 20.242 | 65.850 | 4.188 | 1.00 | 50.00 | C |
| ATOM | 9019 | O | CYS | F | 393 | 21.054 | 66.731 | 4.469 | 1.00 | 50.00 | O |
| ATOM | 9020 | CB | CYS | F | 393 | 20.779 | 63.489 | 3.388 | 1.00 | 50.00 | C |
| ATOM | 9021 | SG | CYS | F | 393 | 19.634 | 62.678 | 2.269 | 1.00 | 50.00 | S |
| ATOM | 9022 | N | LYS | F | 394 | 19.112 | 65.592 | 4.824 | 1.00 | 50.00 | N |
| ATOM | 9023 | CA | LYS | F | 394 | 18.656 | 66.385 | 5.962 | 1.00 | 50.00 | C |
| ATOM | 9024 | C | LYS | F | 394 | 18.557 | 65.430 | 7.140 | 1.00 | 50.00 | C |
| ATOM | 9025 | O | LYS | F | 394 | 17.565 | 64.774 | 7.456 | 1.00 | 50.00 | O |
| ATOM | 9026 | CB | LYS | F | 394 | 17.368 | 67.146 | 5.663 | 1.00 | 50.00 | C |
| ATOM | 9027 | CG | LYS | F | 394 | 17.638 | 68.200 | 4.592 | 1.00 | 50.00 | C |
| ATOM | 9028 | CD | LYS | F | 394 | 16.440 | 69.133 | 4.453 | 1.00 | 50.00 | C |
| ATOM | 9029 | CE | LYS | F | 394 | 16.744 | 70.143 | 3.351 | 1.00 | 50.00 | C |
| ATOM | 9030 | NZ | LYS | F | 394 | 15.540 | 70.952 | 3.118 | 1.00 | 50.00 | N |
| ATOM | 9031 | N | ILE | F | 395 | 19.706 | 65.473 | 7.789 | 1.00 | 50.00 | N |
| ATOM | 9032 | CA | ILE | F | 395 | 19.994 | 64.926 | 9.111 | 1.00 | 50.00 | C |
| ATOM | 9033 | C | ILE | F | 395 | 19.138 | 65.623 | 10.167 | 1.00 | 50.00 | C |
| ATOM | 9034 | O | ILE | F | 395 | 18.960 | 66.840 | 10.110 | 1.00 | 50.00 | O |
| ATOM | 9035 | CB | ILE | F | 395 | 21.517 | 65.105 | 9.147 | 1.00 | 50.00 | C |
| ATOM | 9036 | CG1 | ILE | F | 395 | 22.328 | 64.230 | 8.187 | 1.00 | 50.00 | C |
| ATOM | 9037 | CG2 | ILE | F | 395 | 21.999 | 64.803 | 10.561 | 1.00 | 50.00 | C |
| ATOM | 9038 | CD1 | ILE | F | 395 | 23.793 | 63.886 | 8.456 | 1.00 | 50.00 | C |
| ATOM | 9039 | N | MET | F | 396 | 18.626 | 64.883 | 11.138 | 1.00 | 50.00 | N |
| ATOM | 9040 | CA | MET | F | 396 | 17.925 | 65.428 | 12.303 | 1.00 | 50.00 | C |
| ATOM | 9041 | C | MET | F | 396 | 18.090 | 64.601 | 13.571 | 1.00 | 50.00 | C |
| ATOM | 9042 | O | MET | F | 396 | 18.533 | 65.167 | 14.569 | 1.00 | 50.00 | O |
| ATOM | 9043 | CB | MET | F | 396 | 16.431 | 65.689 | 12.103 | 1.00 | 50.00 | C |
| ATOM | 9044 | CG | MET | F | 396 | 16.109 | 67.076 | 11.555 | 1.00 | 50.00 | C |
| ATOM | 9045 | SD | MET | F | 396 | 14.327 | 67.265 | 11.388 | 1.00 | 50.00 | S |
| ATOM | 9046 | CE | MET | F | 396 | 14.373 | 68.887 | 10.610 | 1.00 | 50.00 | C |
| ATOM | 9047 | N | THR | F | 397 | 17.629 | 63.361 | 13.609 | 1.00 | 50.00 | N |
| ATOM | 9048 | CA | THR | F | 397 | 17.556 | 62.514 | 14.800 | 1.00 | 50.00 | C |
| ATOM | 9049 | C | THR | F | 397 | 16.915 | 61.203 | 14.365 | 1.00 | 50.00 | C |
| ATOM | 9050 | O | THR | F | 397 | 15.978 | 61.179 | 13.567 | 1.00 | 50.00 | O |
| ATOM | 9051 | CB | THR | F | 397 | 16.851 | 63.044 | 16.052 | 1.00 | 50.00 | C |
| ATOM | 9052 | OG1 | THR | F | 397 | 17.087 | 62.153 | 17.134 | 1.00 | 50.00 | O |
| ATOM | 9053 | CG2 | THR | F | 397 | 15.351 | 63.313 | 15.965 | 1.00 | 50.00 | C |
| ATOM | 9054 | N | SER | F | 398 | 17.390 | 60.109 | 14.938 | 1.00 | 50.00 | N |
| ATOM | 9055 | CA | SER | F | 398 | 16.762 | 58.806 | 14.726 | 1.00 | 50.00 | C |
| ATOM | 9056 | C | SER | F | 398 | 15.509 | 58.782 | 15.588 | 1.00 | 50.00 | C |
| ATOM | 9057 | O | SER | F | 398 | 15.288 | 59.560 | 16.516 | 1.00 | 50.00 | O |
| ATOM | 9058 | CB | SER | F | 398 | 17.671 | 57.600 | 14.957 | 1.00 | 50.00 | C |
| ATOM | 9059 | OG | SER | F | 398 | 18.173 | 57.545 | 16.284 | 1.00 | 50.00 | O |
| ATOM | 9060 | N | LYS | F | 399 | 14.669 | 57.822 | 15.247 | 1.00 | 50.00 | N |
| ATOM | 9061 | CA | LYS | F | 399 | 13.440 | 57.570 | 15.997 | 1.00 | 50.00 | C |
| ATOM | 9062 | C | LYS | F | 399 | 13.608 | 56.887 | 17.350 | 1.00 | 50.00 | C |
| ATOM | 9063 | O | LYS | F | 399 | 12.632 | 56.297 | 17.810 | 1.00 | 50.00 | O |
| ATOM | 9064 | CB | LYS | F | 399 | 12.571 | 56.804 | 14.999 | 1.00 | 50.00 | C |
| ATOM | 9065 | CG | LYS | F | 399 | 12.009 | 57.738 | 13.930 | 1.00 | 50.00 | C |
| ATOM | 9066 | CD | LYS | F | 399 | 10.999 | 57.072 | 13.000 | 1.00 | 50.00 | C |
| ATOM | 9067 | CE | LYS | F | 399 | 10.511 | 58.108 | 11.990 | 1.00 | 50.00 | C |
| ATOM | 9068 | NZ | LYS | F | 399 | 9.456 | 57.531 | 11.146 | 1.00 | 50.00 | N |
| ATOM | 9069 | N | THR | F | 400 | 14.783 | 56.869 | 17.962 | 1.00 | 50.00 | N |
| ATOM | 9070 | CA | THR | F | 400 | 15.324 | 55.670 | 18.602 | 1.00 | 50.00 | C |
| ATOM | 9071 | C | THR | F | 400 | 16.364 | 56.105 | 19.629 | 1.00 | 50.00 | C |
| ATOM | 9072 | O | THR | F | 400 | 17.306 | 56.809 | 19.265 | 1.00 | 50.00 | O |
| ATOM | 9073 | CB | THR | F | 400 | 15.881 | 54.646 | 17.608 | 1.00 | 50.00 | C |
| ATOM | 9074 | OG1 | THR | F | 400 | 17.181 | 54.970 | 17.138 | 1.00 | 50.00 | O |
| ATOM | 9075 | CG2 | THR | F | 400 | 15.097 | 54.387 | 16.322 | 1.00 | 50.00 | C |
| ATOM | 9076 | N | ASP | F | 401 | 16.252 | 55.694 | 20.882 | 1.00 | 50.00 | N |
| ATOM | 9077 | CA | ASP | F | 401 | 17.162 | 56.124 | 21.946 | 1.00 | 50.00 | C |
| ATOM | 9078 | C | ASP | F | 401 | 18.606 | 55.663 | 21.774 | 1.00 | 50.00 | C |
| ATOM | 9079 | O | ASP | F | 401 | 19.507 | 56.499 | 21.724 | 1.00 | 50.00 | O |
| ATOM | 9080 | CB | ASP | F | 401 | 16.625 | 55.806 | 23.343 | 1.00 | 50.00 | C |
| ATOM | 9081 | CG | ASP | F | 401 | 17.311 | 56.561 | 24.473 | 1.00 | 50.00 | C |
| ATOM | 9082 | OD1 | ASP | F | 401 | 17.124 | 57.793 | 24.579 | 1.00 | 50.00 | O |
| ATOM | 9083 | OD2 | ASP | F | 401 | 18.063 | 55.938 | 25.254 | 1.00 | 50.00 | O |
| ATOM | 9084 | N | VAL | F | 402 | 18.820 | 54.358 | 21.732 | 1.00 | 50.00 | N |
| ATOM | 9085 | CA | VAL | F | 402 | 20.135 | 53.724 | 21.628 | 1.00 | 50.00 | C |
| ATOM | 9086 | C | VAL | F | 402 | 19.868 | 52.458 | 20.822 | 1.00 | 50.00 | C |
| ATOM | 9087 | O | VAL | F | 402 | 18.945 | 51.687 | 21.083 | 1.00 | 50.00 | O |
| ATOM | 9088 | CB | VAL | F | 402 | 20.805 | 53.408 | 22.969 | 1.00 | 50.00 | C |
| ATOM | 9089 | CG1 | VAL | F | 402 | 22.067 | 52.549 | 22.881 | 1.00 | 50.00 | C |
| ATOM | 9090 | CG2 | VAL | F | 402 | 21.178 | 54.658 | 23.764 | 1.00 | 50.00 | C |
| ATOM | 9091 | N | SER | F | 403 | 20.784 | 52.257 | 19.892 | 1.00 | 50.00 | N |
| ATOM | 9092 | CA | SER | F | 403 | 21.022 | 51.040 | 19.116 | 1.00 | 50.00 | C |
| ATOM | 9093 | C | SER | F | 403 | 21.917 | 51.577 | 18.003 | 1.00 | 50.00 | C |
| ATOM | 9094 | O | SER | F | 403 | 21.439 | 51.652 | 16.871 | 1.00 | 50.00 | O |
| ATOM | 9095 | CB | SER | F | 403 | 19.769 | 50.324 | 18.605 | 1.00 | 50.00 | C |
| ATOM | 9096 | OG | SER | F | 403 | 20.061 | 49.052 | 18.047 | 1.00 | 50.00 | O |
| ATOM | 9097 | N | SER | F | 404 | 23.153 | 51.998 | 18.233 | 1.00 | 50.00 | N |
| ATOM | 9098 | CA | SER | F | 404 | 24.003 | 52.552 | 17.176 | 1.00 | 50.00 | C |
| ATOM | 9099 | C | SER | F | 404 | 24.707 | 51.432 | 16.417 | 1.00 | 50.00 | C |
| ATOM | 9100 | O | SER | F | 404 | 25.912 | 51.207 | 16.524 | 1.00 | 50.00 | O |
| ATOM | 9101 | CB | SER | F | 404 | 24.972 | 53.602 | 17.715 | 1.00 | 50.00 | C |
| ATOM | 9102 | OG | SER | F | 404 | 24.312 | 54.787 | 18.137 | 1.00 | 50.00 | O |
| ATOM | 9103 | N | SER | F | 405 | 23.895 | 50.679 | 15.695 | 1.00 | 50.00 | N |
| ATOM | 9104 | CA | SER | F | 405 | 23.952 | 49.221 | 15.592 | 1.00 | 50.00 | C |
| ATOM | 9105 | C | SER | F | 405 | 22.562 | 48.685 | 15.275 | 1.00 | 50.00 | C |
| ATOM | 9106 | O | SER | F | 405 | 21.586 | 48.990 | 15.959 | 1.00 | 50.00 | O |
| ATOM | 9107 | CB | SER | F | 405 | 24.634 | 48.404 | 16.690 | 1.00 | 50.00 | C |
| ATOM | 9108 | OG | SER | F | 405 | 23.860 | 48.294 | 17.875 | 1.00 | 50.00 | O |
| ATOM | 9109 | N | VAL | F | 406 | 22.462 | 47.876 | 14.234 | 1.00 | 50.00 | N |
| ATOM | 9110 | CA | VAL | F | 406 | 21.224 | 47.264 | 13.750 | 1.00 | 50.00 | C |
| ATOM | 9111 | C | VAL | F | 406 | 21.636 | 45.909 | 13.186 | 1.00 | 50.00 | C |
| ATOM | 9112 | O | VAL | F | 406 | 22.681 | 45.752 | 12.554 | 1.00 | 50.00 | O |
| ATOM | 9113 | CB | VAL | F | 406 | 20.495 | 48.130 | 12.718 | 1.00 | 50.00 | C |
| ATOM | 9114 | CG1 | VAL | F | 406 | 19.378 | 47.442 | 11.934 | 1.00 | 50.00 | C |
| ATOM | 9115 | CG2 | VAL | F | 406 | 19.868 | 49.390 | 13.313 | 1.00 | 50.00 | C |
| ATOM | 9116 | N | ILE | F | 407 | 20.731 | 44.951 | 13.300 | 1.00 | 50.00 | N |
| ATOM | 9117 | CA | ILE | F | 407 | 20.807 | 43.691 | 12.561 | 1.00 | 50.00 | C |
| ATOM | 9118 | C | ILE | F | 407 | 20.275 | 43.758 | 11.133 | 1.00 | 50.00 | C |
| ATOM | 9119 | O | ILE | F | 407 | 19.256 | 44.391 | 10.861 | 1.00 | 50.00 | O |
| ATOM | 9120 | CB | ILE | F | 407 | 20.177 | 42.580 | 13.404 | 1.00 | 50.00 | C |
| ATOM | 9121 | CG1 | ILE | F | 407 | 20.965 | 41.289 | 13.193 | 1.00 | 50.00 | C |
| ATOM | 9122 | CG2 | ILE | F | 407 | 18.674 | 42.378 | 13.206 | 1.00 | 50.00 | C |
| ATOM | 9123 | CD1 | ILE | F | 407 | 22.313 | 41.223 | 13.910 | 1.00 | 50.00 | C |
| ATOM | 9124 | N | THR | F | 408 | 20.893 | 43.001 | 10.242 | 1.00 | 50.00 | N |
| ATOM | 9125 | CA | THR | F | 408 | 20.457 | 42.814 | 8.858 | 1.00 | 50.00 | C |
| ATOM | 9126 | C | THR | F | 408 | 21.074 | 41.505 | 8.384 | 1.00 | 50.00 | C |
| ATOM | 9127 | O | THR | F | 408 | 22.301 | 41.398 | 8.379 | 1.00 | 50.00 | O |
| ATOM | 9128 | CB | THR | F | 408 | 20.917 | 43.971 | 7.969 | 1.00 | 50.00 | C |
| ATOM | 9129 | OG1 | THR | F | 408 | 20.204 | 45.152 | 8.306 | 1.00 | 50.00 | O |
| ATOM | 9130 | CG2 | THR | F | 408 | 20.791 | 43.779 | 6.459 | 1.00 | 50.00 | C |
| ATOM | 9131 | N | SER | F | 409 | 20.254 | 40.596 | 7.880 | 1.00 | 50.00 | N |
| ATOM | 9132 | CA | SER | F | 409 | 20.666 | 39.421 | 7.109 | 1.00 | 50.00 | C |
| ATOM | 9133 | C | SER | F | 409 | 21.908 | 38.726 | 7.649 | 1.00 | 50.00 | C |
| ATOM | 9134 | O | SER | F | 409 | 22.981 | 38.920 | 7.080 | 1.00 | 50.00 | O |
| ATOM | 9135 | CB | SER | F | 409 | 20.871 | 39.743 | 5.628 | 1.00 | 50.00 | C |
| ATOM | 9136 | OG | SER | F | 409 | 19.682 | 40.166 | 4.978 | 1.00 | 50.00 | O |
| ATOM | 9137 | N | LEU | F | 410 | 21.836 | 38.059 | 8.789 | 1.00 | 50.00 | N |
| ATOM | 9138 | CA | LEU | F | 410 | 23.012 | 37.447 | 9.408 | 1.00 | 50.00 | C |
| ATOM | 9139 | C | LEU | F | 410 | 24.140 | 38.365 | 9.872 | 1.00 | 50.00 | C |
| ATOM | 9140 | O | LEU | F | 410 | 25.312 | 37.998 | 9.798 | 1.00 | 50.00 | O |
| ATOM | 9141 | CB | LEU | F | 410 | 23.402 | 36.208 | 8.596 | 1.00 | 50.00 | C |
| ATOM | 9142 | CG | LEU | F | 410 | 24.262 | 36.239 | 7.329 | 1.00 | 50.00 | C |
| ATOM | 9143 | CD1 | LEU | F | 410 | 25.418 | 37.220 | 7.126 | 1.00 | 50.00 | C |
| ATOM | 9144 | CD2 | LEU | F | 410 | 24.901 | 34.861 | 7.172 | 1.00 | 50.00 | C |
| ATOM | 9145 | N | GLY | F | 411 | 23.898 | 39.568 | 10.366 | 1.00 | 50.00 | N |
| ATOM | 9146 | CA | GLY | F | 411 | 24.962 | 40.571 | 10.388 | 1.00 | 50.00 | C |
| ATOM | 9147 | C | GLY | F | 411 | 24.615 | 41.857 | 11.120 | 1.00 | 50.00 | C |
| ATOM | 9148 | O | GLY | F | 411 | 23.445 | 42.229 | 11.182 | 1.00 | 50.00 | O |
| ATOM | 9149 | N | ALA | F | 412 | 25.624 | 42.535 | 11.638 | 1.00 | 50.00 | N |
| ATOM | 9150 | CA | ALA | F | 412 | 25.420 | 43.693 | 12.508 | 1.00 | 50.00 | C |
| ATOM | 9151 | C | ALA | F | 412 | 26.110 | 44.915 | 11.919 | 1.00 | 50.00 | C |
| ATOM | 9152 | O | ALA | F | 412 | 27.300 | 45.116 | 12.157 | 1.00 | 50.00 | O |
| ATOM | 9153 | CB | ALA | F | 412 | 25.887 | 43.327 | 13.916 | 1.00 | 50.00 | C |
| ATOM | 9154 | N | ILE | F | 413 | 25.381 | 45.751 | 11.197 | 1.00 | 50.00 | N |
| ATOM | 9155 | CA | ILE | F | 413 | 25.824 | 47.079 | 10.762 | 1.00 | 50.00 | C |
| ATOM | 9156 | C | ILE | F | 413 | 25.845 | 48.002 | 11.974 | 1.00 | 50.00 | C |
| ATOM | 9157 | O | ILE | F | 413 | 24.803 | 48.152 | 12.611 | 1.00 | 50.00 | O |
| ATOM | 9158 | CB | ILE | F | 413 | 24.922 | 47.609 | 9.643 | 1.00 | 50.00 | C |
| ATOM | 9159 | CG1 | ILE | F | 413 | 25.189 | 46.912 | 8.308 | 1.00 | 50.00 | C |
| ATOM | 9160 | CG2 | ILE | F | 413 | 24.925 | 49.121 | 9.414 | 1.00 | 50.00 | C |
| ATOM | 9161 | CD1 | ILE | F | 413 | 23.922 | 46.390 | 7.636 | 1.00 | 50.00 | C |
| ATOM | 9162 | N | VAL | F | 414 | 26.977 | 48.617 | 12.274 | 1.00 | 50.00 | N |
| ATOM | 9163 | CA | VAL | F | 414 | 27.177 | 49.342 | 13.530 | 1.00 | 50.00 | C |
| ATOM | 9164 | C | VAL | F | 414 | 27.944 | 50.634 | 13.271 | 1.00 | 50.00 | C |
| ATOM | 9165 | O | VAL | F | 414 | 28.928 | 50.622 | 12.532 | 1.00 | 50.00 | O |
| ATOM | 9166 | CB | VAL | F | 414 | 27.701 | 48.450 | 14.661 | 1.00 | 50.00 | C |
| ATOM | 9167 | CG1 | VAL | F | 414 | 27.184 | 47.017 | 14.793 | 1.00 | 50.00 | C |
| ATOM | 9168 | CG2 | VAL | F | 414 | 29.220 | 48.301 | 14.696 | 1.00 | 50.00 | C |
| ATOM | 9169 | N | SER | F | 415 | 27.588 | 51.722 | 13.935 | 1.00 | 50.00 | N |
| ATOM | 9170 | CA | SER | F | 415 | 28.413 | 52.929 | 14.005 | 1.00 | 50.00 | C |
| ATOM | 9171 | C | SER | F | 415 | 29.536 | 52.740 | 15.020 | 1.00 | 50.00 | C |
| ATOM | 9172 | O | SER | F | 415 | 29.410 | 53.111 | 16.187 | 1.00 | 50.00 | O |
| ATOM | 9173 | CB | SER | F | 415 | 27.557 | 54.155 | 14.324 | 1.00 | 50.00 | C |
| ATOM | 9174 | OG | SER | F | 415 | 26.575 | 54.401 | 13.329 | 1.00 | 50.00 | O |
| ATOM | 9175 | N | CYS | F | 416 | 30.629 | 52.135 | 14.581 | 1.00 | 50.00 | N |
| ATOM | 9176 | CA | CYS | F | 416 | 31.747 | 51.741 | 15.439 | 1.00 | 50.00 | C |
| ATOM | 9177 | C | CYS | F | 416 | 32.488 | 52.889 | 16.111 | 1.00 | 50.00 | C |
| ATOM | 9178 | O | CYS | F | 416 | 32.478 | 52.989 | 17.337 | 1.00 | 50.00 | O |
| ATOM | 9179 | CB | CYS | F | 416 | 32.722 | 50.833 | 14.692 | 1.00 | 50.00 | C |
| ATOM | 9180 | SG | CYS | F | 416 | 32.065 | 49.160 | 14.613 | 1.00 | 50.00 | S |
| ATOM | 9181 | N | TYR | F | 417 | 33.095 | 53.755 | 15.318 | 1.00 | 50.00 | N |
| ATOM | 9182 | CA | TYR | F | 417 | 33.659 | 55.020 | 15.787 | 1.00 | 50.00 | C |
| ATOM | 9183 | C | TYR | F | 417 | 32.647 | 56.062 | 16.252 | 1.00 | 50.00 | C |
| ATOM | 9184 | O | TYR | F | 417 | 32.945 | 56.742 | 17.233 | 1.00 | 50.00 | O |
| ATOM | 9185 | CB | TYR | F | 417 | 34.580 | 55.496 | 14.663 | 1.00 | 50.00 | C |
| ATOM | 9186 | CG | TYR | F | 417 | 35.407 | 56.719 | 14.993 | 1.00 | 50.00 | C |
| ATOM | 9187 | CD1 | TYR | F | 417 | 36.511 | 56.612 | 15.762 | 1.00 | 50.00 | C |
| ATOM | 9188 | CD2 | TYR | F | 417 | 34.970 | 57.939 | 14.616 | 1.00 | 50.00 | C |
| ATOM | 9189 | CE1 | TYR | F | 417 | 37.164 | 57.719 | 16.167 | 1.00 | 50.00 | C |
| ATOM | 9190 | CE2 | TYR | F | 417 | 35.616 | 59.048 | 15.029 | 1.00 | 50.00 | C |
| ATOM | 9191 | CZ | TYR | F | 417 | 36.715 | 58.937 | 15.801 | 1.00 | 50.00 | C |
| ATOM | 9192 | OH | TYR | F | 417 | 37.370 | 60.074 | 16.197 | 1.00 | 50.00 | O |
| ATOM | 9193 | N | GLY | F | 418 | 31.505 | 56.212 | 15.599 | 1.00 | 50.00 | N |
| ATOM | 9194 | CA | GLY | F | 418 | 30.437 | 57.126 | 16.007 | 1.00 | 50.00 | C |
| ATOM | 9195 | C | GLY | F | 418 | 29.977 | 57.028 | 17.456 | 1.00 | 50.00 | C |
| ATOM | 9196 | O | GLY | F | 418 | 30.226 | 57.950 | 18.232 | 1.00 | 50.00 | O |
| ATOM | 9197 | N | LYS | F | 419 | 29.414 | 55.892 | 17.831 | 1.00 | 50.00 | N |
| ATOM | 9198 | CA | LYS | F | 419 | 29.193 | 55.531 | 19.231 | 1.00 | 50.00 | C |
| ATOM | 9199 | C | LYS | F | 419 | 30.444 | 55.335 | 20.081 | 1.00 | 50.00 | C |
| ATOM | 9200 | O | LYS | F | 419 | 30.500 | 55.991 | 21.121 | 1.00 | 50.00 | O |
| ATOM | 9201 | CB | LYS | F | 419 | 28.293 | 54.297 | 19.296 | 1.00 | 50.00 | C |
| ATOM | 9202 | CG | LYS | F | 419 | 27.718 | 53.976 | 20.676 | 1.00 | 50.00 | C |
| ATOM | 9203 | CD | LYS | F | 419 | 26.439 | 54.761 | 20.962 | 1.00 | 50.00 | C |
| ATOM | 9204 | CE | LYS | F | 419 | 25.851 | 54.551 | 22.354 | 1.00 | 50.00 | C |
| ATOM | 9205 | NZ | LYS | F | 419 | 24.576 | 55.279 | 22.433 | 1.00 | 50.00 | N |
| ATOM | 9206 | N | THR | F | 420 | 31.345 | 54.440 | 19.703 | 1.00 | 50.00 | N |
| ATOM | 9207 | CA | THR | F | 420 | 32.358 | 53.758 | 20.516 | 1.00 | 50.00 | C |
| ATOM | 9208 | C | THR | F | 420 | 32.253 | 52.233 | 20.490 | 1.00 | 50.00 | C |
| ATOM | 9209 | O | THR | F | 420 | 33.140 | 51.580 | 21.039 | 1.00 | 50.00 | O |
| ATOM | 9210 | CB | THR | F | 420 | 32.687 | 54.352 | 21.889 | 1.00 | 50.00 | C |
| ATOM | 9211 | OG1 | THR | F | 420 | 33.057 | 55.716 | 21.742 | 1.00 | 50.00 | O |
| ATOM | 9212 | CG2 | THR | F | 420 | 33.817 | 53.770 | 22.736 | 1.00 | 50.00 | C |
| ATOM | 9213 | N | LYS | F | 421 | 31.291 | 51.590 | 19.846 | 1.00 | 50.00 | N |
| ATOM | 9214 | CA | LYS | F | 421 | 30.920 | 50.181 | 19.996 | 1.00 | 50.00 | C |
| ATOM | 9215 | C | LYS | F | 421 | 31.978 | 49.103 | 20.213 | 1.00 | 50.00 | C |
| ATOM | 9216 | O | LYS | F | 421 | 31.958 | 48.444 | 21.252 | 1.00 | 50.00 | O |
| ATOM | 9217 | CB | LYS | F | 421 | 29.958 | 49.799 | 18.869 | 1.00 | 50.00 | C |
| ATOM | 9218 | CG | LYS | F | 421 | 28.542 | 50.372 | 18.853 | 1.00 | 50.00 | C |
| ATOM | 9219 | CD | LYS | F | 421 | 27.596 | 49.791 | 19.902 | 1.00 | 50.00 | C |
| ATOM | 9220 | CE | LYS | F | 421 | 26.216 | 50.443 | 19.855 | 1.00 | 50.00 | C |
| ATOM | 9221 | NZ | LYS | F | 421 | 25.452 | 50.160 | 21.078 | 1.00 | 50.00 | N |
| ATOM | 9222 | N | CYS | F | 422 | 32.830 | 48.785 | 19.252 | 1.00 | 50.00 | N |
| ATOM | 9223 | CA | CYS | F | 422 | 32.799 | 47.433 | 18.696 | 1.00 | 50.00 | C |
| ATOM | 9224 | C | CYS | F | 422 | 33.984 | 46.557 | 19.083 | 1.00 | 50.00 | C |
| ATOM | 9225 | O | CYS | F | 422 | 35.001 | 46.513 | 18.393 | 1.00 | 50.00 | O |
| ATOM | 9226 | CB | CYS | F | 422 | 32.742 | 47.573 | 17.177 | 1.00 | 50.00 | C |
| ATOM | 9227 | SG | CYS | F | 422 | 31.504 | 48.663 | 16.467 | 1.00 | 50.00 | S |
| ATOM | 9228 | N | THR | F | 423 | 33.850 | 45.786 | 20.149 | 1.00 | 50.00 | N |
| ATOM | 9229 | CA | THR | F | 423 | 34.833 | 44.776 | 20.543 | 1.00 | 50.00 | C |
| ATOM | 9230 | C | THR | F | 423 | 34.543 | 43.472 | 19.809 | 1.00 | 50.00 | C |
| ATOM | 9231 | O | THR | F | 423 | 33.467 | 42.897 | 19.966 | 1.00 | 50.00 | O |
| ATOM | 9232 | CB | THR | F | 423 | 34.807 | 44.640 | 22.067 | 1.00 | 50.00 | C |
| ATOM | 9233 | OG1 | THR | F | 423 | 35.274 | 45.845 | 22.656 | 1.00 | 50.00 | O |
| ATOM | 9234 | CG2 | THR | F | 423 | 35.643 | 43.504 | 22.654 | 1.00 | 50.00 | C |
| ATOM | 9235 | N | ALA | F | 424 | 35.516 | 42.958 | 19.074 | 1.00 | 50.00 | N |
| ATOM | 9236 | CA | ALA | F | 424 | 35.486 | 41.588 | 18.562 | 1.00 | 50.00 | C |
| ATOM | 9237 | C | ALA | F | 424 | 36.202 | 40.662 | 19.540 | 1.00 | 50.00 | C |
| ATOM | 9238 | O | ALA | F | 424 | 37.429 | 40.678 | 19.639 | 1.00 | 50.00 | O |
| ATOM | 9239 | CB | ALA | F | 424 | 36.097 | 41.578 | 17.162 | 1.00 | 50.00 | C |
| ATOM | 9240 | N | SER | F | 425 | 35.440 | 39.850 | 20.254 | 1.00 | 50.00 | N |
| ATOM | 9241 | CA | SER | F | 425 | 35.958 | 38.804 | 21.139 | 1.00 | 50.00 | C |
| ATOM | 9242 | C | SER | F | 425 | 36.708 | 37.628 | 20.517 | 1.00 | 50.00 | C |
| ATOM | 9243 | O | SER | F | 425 | 37.254 | 36.832 | 21.281 | 1.00 | 50.00 | O |
| ATOM | 9244 | CB | SER | F | 425 | 34.828 | 38.342 | 22.060 | 1.00 | 50.00 | C |
| ATOM | 9245 | OG | SER | F | 425 | 34.459 | 39.345 | 22.996 | 1.00 | 50.00 | O |
| ATOM | 9246 | N | ASN | F | 426 | 36.807 | 37.484 | 19.204 | 1.00 | 50.00 | N |
| ATOM | 9247 | CA | ASN | F | 426 | 37.589 | 36.439 | 18.541 | 1.00 | 50.00 | C |
| ATOM | 9248 | C | ASN | F | 426 | 39.086 | 36.681 | 18.706 | 1.00 | 50.00 | C |
| ATOM | 9249 | O | ASN | F | 426 | 39.562 | 37.762 | 19.052 | 1.00 | 50.00 | O |
| ATOM | 9250 | CB | ASN | F | 426 | 37.145 | 36.310 | 17.082 | 1.00 | 50.00 | C |
| ATOM | 9251 | CG | ASN | F | 426 | 37.644 | 35.085 | 16.323 | 1.00 | 50.00 | C |
| ATOM | 9252 | OD1 | ASN | F | 426 | 37.144 | 33.975 | 16.494 | 1.00 | 50.00 | O |
| ATOM | 9253 | ND2 | ASN | F | 426 | 38.662 | 35.220 | 15.493 | 1.00 | 50.00 | N |
| ATOM | 9254 | N | LYS | F | 427 | 39.842 | 35.629 | 18.445 | 1.00 | 50.00 | N |
| ATOM | 9255 | CA | LYS | F | 427 | 41.304 | 35.611 | 18.386 | 1.00 | 50.00 | C |
| ATOM | 9256 | C | LYS | F | 427 | 42.053 | 36.719 | 17.656 | 1.00 | 50.00 | C |
| ATOM | 9257 | O | LYS | F | 427 | 43.025 | 37.206 | 18.233 | 1.00 | 50.00 | O |
| ATOM | 9258 | CB | LYS | F | 427 | 41.725 | 34.244 | 17.844 | 1.00 | 50.00 | C |
| ATOM | 9259 | CG | LYS | F | 427 | 41.377 | 33.058 | 18.742 | 1.00 | 50.00 | C |
| ATOM | 9260 | CD | LYS | F | 427 | 41.938 | 31.739 | 18.215 | 1.00 | 50.00 | C |
| ATOM | 9261 | CE | LYS | F | 427 | 41.564 | 30.567 | 19.119 | 1.00 | 50.00 | C |
| ATOM | 9262 | NZ | LYS | F | 427 | 42.164 | 29.321 | 18.618 | 1.00 | 50.00 | N |
| ATOM | 9263 | N | ASN | F | 428 | 41.678 | 37.127 | 16.453 | 1.00 | 50.00 | N |
| ATOM | 9264 | CA | ASN | F | 428 | 42.430 | 38.095 | 15.650 | 1.00 | 50.00 | C |
| ATOM | 9265 | C | ASN | F | 428 | 42.503 | 39.545 | 16.129 | 1.00 | 50.00 | C |
| ATOM | 9266 | O | ASN | F | 428 | 42.913 | 40.413 | 15.359 | 1.00 | 50.00 | O |
| ATOM | 9267 | CB | ASN | F | 428 | 41.924 | 37.994 | 14.208 | 1.00 | 50.00 | C |
| ATOM | 9268 | CG | ASN | F | 428 | 42.088 | 36.699 | 13.422 | 1.00 | 50.00 | C |
| ATOM | 9269 | OD1 | ASN | F | 428 | 41.111 | 36.077 | 13.007 | 1.00 | 50.00 | O |
| ATOM | 9270 | ND2 | ASN | F | 428 | 43.306 | 36.271 | 13.148 | 1.00 | 50.00 | N |
| ATOM | 9271 | N | ARG | F | 429 | 42.131 | 39.894 | 17.351 | 1.00 | 50.00 | N |
| ATOM | 9272 | CA | ARG | F | 429 | 41.283 | 41.050 | 17.649 | 1.00 | 50.00 | C |
| ATOM | 9273 | C | ARG | F | 429 | 41.372 | 41.489 | 19.109 | 1.00 | 50.00 | C |
| ATOM | 9274 | O | ARG | F | 429 | 41.594 | 40.644 | 19.976 | 1.00 | 50.00 | O |
| ATOM | 9275 | CB | ARG | F | 429 | 39.819 | 40.702 | 17.374 | 1.00 | 50.00 | C |
| ATOM | 9276 | CG | ARG | F | 429 | 39.385 | 40.306 | 15.962 | 1.00 | 50.00 | C |
| ATOM | 9277 | CD | ARG | F | 429 | 39.763 | 41.270 | 14.838 | 1.00 | 50.00 | C |
| ATOM | 9278 | NE | ARG | F | 429 | 39.095 | 42.532 | 15.140 | 1.00 | 50.00 | N |
| ATOM | 9279 | CZ | ARG | F | 429 | 39.112 | 43.610 | 14.380 | 1.00 | 50.00 | C |
| ATOM | 9280 | NH1 | ARG | F | 429 | 39.677 | 43.730 | 13.192 | 1.00 | 50.00 | N |
| ATOM | 9281 | NH2 | ARG | F | 429 | 38.475 | 44.635 | 14.906 | 1.00 | 50.00 | N |
| ATOM | 9282 | N | GLY | F | 430 | 41.139 | 42.700 | 19.592 | 1.00 | 50.00 | N |
| ATOM | 9283 | CA | GLY | F | 430 | 40.963 | 43.937 | 18.830 | 1.00 | 50.00 | C |
| ATOM | 9284 | C | GLY | F | 430 | 39.596 | 44.608 | 18.899 | 1.00 | 50.00 | C |
| ATOM | 9285 | O | GLY | F | 430 | 38.569 | 43.933 | 18.958 | 1.00 | 50.00 | O |
| ATOM | 9286 | N | ILE | F | 431 | 39.585 | 45.930 | 18.815 | 1.00 | 50.00 | N |
| ATOM | 9287 | CA | ILE | F | 431 | 38.393 | 46.751 | 18.583 | 1.00 | 50.00 | C |
| ATOM | 9288 | C | ILE | F | 431 | 38.297 | 47.015 | 17.083 | 1.00 | 50.00 | C |
| ATOM | 9289 | O | ILE | F | 431 | 39.276 | 47.420 | 16.456 | 1.00 | 50.00 | O |
| ATOM | 9290 | CB | ILE | F | 431 | 38.364 | 48.047 | 19.402 | 1.00 | 50.00 | C |
| ATOM | 9291 | CG1 | ILE | F | 431 | 38.364 | 47.813 | 20.914 | 1.00 | 50.00 | C |
| ATOM | 9292 | CG2 | ILE | F | 431 | 37.209 | 48.987 | 19.050 | 1.00 | 50.00 | C |
| ATOM | 9293 | CD1 | ILE | F | 431 | 38.440 | 49.034 | 21.829 | 1.00 | 50.00 | C |
| ATOM | 9294 | N | ILE | F | 432 | 37.108 | 46.877 | 16.518 | 1.00 | 50.00 | N |
| ATOM | 9295 | CA | ILE | F | 432 | 36.809 | 47.250 | 15.134 | 1.00 | 50.00 | C |
| ATOM | 9296 | C | ILE | F | 432 | 36.473 | 48.734 | 15.073 | 1.00 | 50.00 | C |
| ATOM | 9297 | O | ILE | F | 432 | 35.395 | 49.157 | 15.490 | 1.00 | 50.00 | O |
| ATOM | 9298 | CB | ILE | F | 432 | 35.825 | 46.309 | 14.432 | 1.00 | 50.00 | C |
| ATOM | 9299 | CG1 | ILE | F | 432 | 35.892 | 46.419 | 12.908 | 1.00 | 50.00 | C |
| ATOM | 9300 | CG2 | ILE | F | 432 | 34.366 | 46.289 | 14.878 | 1.00 | 50.00 | C |
| ATOM | 9301 | CD1 | ILE | F | 432 | 36.549 | 45.220 | 12.230 | 1.00 | 50.00 | C |
| ATOM | 9302 | N | LYS | F | 433 | 37.418 | 49.516 | 14.580 | 1.00 | 50.00 | N |
| ATOM | 9303 | CA | LYS | F | 433 | 37.296 | 50.969 | 14.463 | 1.00 | 50.00 | C |
| ATOM | 9304 | C | LYS | F | 433 | 37.081 | 51.364 | 13.007 | 1.00 | 50.00 | C |
| ATOM | 9305 | O | LYS | F | 433 | 37.537 | 50.707 | 12.071 | 1.00 | 50.00 | O |
| ATOM | 9306 | CB | LYS | F | 433 | 38.529 | 51.647 | 15.061 | 1.00 | 50.00 | C |
| ATOM | 9307 | CG | LYS | F | 433 | 38.666 | 51.489 | 16.575 | 1.00 | 50.00 | C |
| ATOM | 9308 | CD | LYS | F | 433 | 39.830 | 52.282 | 17.164 | 1.00 | 50.00 | C |
| ATOM | 9309 | CE | LYS | F | 433 | 39.989 | 52.040 | 18.663 | 1.00 | 50.00 | C |
| ATOM | 9310 | NZ | LYS | F | 433 | 41.092 | 52.848 | 19.204 | 1.00 | 50.00 | N |
| ATOM | 9311 | N | THR | F | 434 | 36.381 | 52.473 | 12.835 | 1.00 | 50.00 | N |
| ATOM | 9312 | CA | THR | F | 434 | 36.049 | 53.043 | 11.529 | 1.00 | 50.00 | C |
| ATOM | 9313 | C | THR | F | 434 | 36.667 | 54.435 | 11.465 | 1.00 | 50.00 | C |
| ATOM | 9314 | O | THR | F | 434 | 36.347 | 55.312 | 12.266 | 1.00 | 50.00 | O |
| ATOM | 9315 | CB | THR | F | 434 | 34.530 | 53.013 | 11.352 | 1.00 | 50.00 | C |
| ATOM | 9316 | OG1 | THR | F | 434 | 34.119 | 51.655 | 11.289 | 1.00 | 50.00 | O |
| ATOM | 9317 | CG2 | THR | F | 434 | 33.978 | 53.682 | 10.095 | 1.00 | 50.00 | C |
| ATOM | 9318 | N | PHE | F | 435 | 37.494 | 54.681 | 10.462 | 1.00 | 50.00 | N |
| ATOM | 9319 | CA | PHE | F | 435 | 37.933 | 56.030 | 10.101 | 1.00 | 50.00 | C |
| ATOM | 9320 | C | PHE | F | 435 | 36.818 | 56.843 | 9.447 | 1.00 | 50.00 | C |
| ATOM | 9321 | O | PHE | F | 435 | 35.791 | 56.316 | 9.020 | 1.00 | 50.00 | O |
| ATOM | 9322 | CB | PHE | F | 435 | 39.222 | 55.884 | 9.289 | 1.00 | 50.00 | C |
| ATOM | 9323 | CG | PHE | F | 435 | 39.979 | 57.165 | 9.003 | 1.00 | 50.00 | C |
| ATOM | 9324 | CD1 | PHE | F | 435 | 40.585 | 57.833 | 10.007 | 1.00 | 50.00 | C |
| ATOM | 9325 | CD2 | PHE | F | 435 | 39.878 | 57.746 | 7.789 | 1.00 | 50.00 | C |
| ATOM | 9326 | CE1 | PHE | F | 435 | 41.027 | 59.093 | 9.819 | 1.00 | 50.00 | C |
| ATOM | 9327 | CE2 | PHE | F | 435 | 40.311 | 59.008 | 7.598 | 1.00 | 50.00 | C |
| ATOM | 9328 | CZ | PHE | F | 435 | 40.879 | 59.685 | 8.617 | 1.00 | 50.00 | C |
| ATOM | 9329 | N | SER | F | 436 | 37.013 | 58.147 | 9.335 | 1.00 | 50.00 | N |
| ATOM | 9330 | CA | SER | F | 436 | 36.189 | 59.049 | 8.529 | 1.00 | 50.00 | C |
| ATOM | 9331 | C | SER | F | 436 | 36.003 | 58.618 | 7.078 | 1.00 | 50.00 | C |
| ATOM | 9332 | O | SER | F | 436 | 36.902 | 58.024 | 6.484 | 1.00 | 50.00 | O |
| ATOM | 9333 | CB | SER | F | 436 | 36.824 | 60.439 | 8.555 | 1.00 | 50.00 | C |
| ATOM | 9334 | OG | SER | F | 436 | 36.853 | 60.990 | 9.864 | 1.00 | 50.00 | O |
| ATOM | 9335 | N | ASN | F | 437 | 34.831 | 58.893 | 6.527 | 1.00 | 50.00 | N |
| ATOM | 9336 | CA | ASN | F | 437 | 34.369 | 58.391 | 5.231 | 1.00 | 50.00 | C |
| ATOM | 9337 | C | ASN | F | 437 | 33.537 | 57.114 | 5.283 | 1.00 | 50.00 | C |
| ATOM | 9338 | O | ASN | F | 437 | 32.590 | 57.008 | 4.505 | 1.00 | 50.00 | O |
| ATOM | 9339 | CB | ASN | F | 437 | 35.324 | 58.392 | 4.035 | 1.00 | 50.00 | C |
| ATOM | 9340 | CG | ASN | F | 437 | 36.029 | 59.707 | 3.729 | 1.00 | 50.00 | C |
| ATOM | 9341 | OD1 | ASN | F | 437 | 37.252 | 59.810 | 3.816 | 1.00 | 50.00 | O |
| ATOM | 9342 | ND2 | ASN | F | 437 | 35.288 | 60.745 | 3.386 | 1.00 | 50.00 | N |
| ATOM | 9343 | N | GLY | F | 438 | 33.814 | 56.173 | 6.172 | 1.00 | 50.00 | N |
| ATOM | 9344 | CA | GLY | F | 438 | 32.915 | 55.058 | 6.467 | 1.00 | 50.00 | C |
| ATOM | 9345 | C | GLY | F | 438 | 31.464 | 55.354 | 6.829 | 1.00 | 50.00 | C |
| ATOM | 9346 | O | GLY | F | 438 | 30.613 | 54.693 | 6.234 | 1.00 | 50.00 | O |
| ATOM | 9347 | N | CYS | F | 439 | 31.161 | 56.228 | 7.779 | 1.00 | 50.00 | N |
| ATOM | 9348 | CA | CYS | F | 439 | 29.884 | 56.300 | 8.496 | 1.00 | 50.00 | C |
| ATOM | 9349 | C | CYS | F | 439 | 29.709 | 55.161 | 9.497 | 1.00 | 50.00 | C |
| ATOM | 9350 | O | CYS | F | 439 | 29.341 | 55.393 | 10.649 | 1.00 | 50.00 | O |
| ATOM | 9351 | CB | CYS | F | 439 | 28.547 | 56.432 | 7.758 | 1.00 | 50.00 | C |
| ATOM | 9352 | SG | CYS | F | 439 | 27.802 | 58.000 | 7.274 | 1.00 | 50.00 | S |
| ATOM | 9353 | N | ASP | F | 440 | 29.810 | 53.933 | 9.013 | 1.00 | 50.00 | N |
| ATOM | 9354 | CA | ASP | F | 440 | 29.135 | 52.745 | 9.532 | 1.00 | 50.00 | C |
| ATOM | 9355 | C | ASP | F | 440 | 29.946 | 51.558 | 9.027 | 1.00 | 50.00 | C |
| ATOM | 9356 | O | ASP | F | 440 | 30.201 | 51.399 | 7.833 | 1.00 | 50.00 | O |
| ATOM | 9357 | CB | ASP | F | 440 | 27.704 | 52.598 | 9.012 | 1.00 | 50.00 | C |
| ATOM | 9358 | CG | ASP | F | 440 | 26.665 | 53.543 | 9.596 | 1.00 | 50.00 | C |
| ATOM | 9359 | OD1 | ASP | F | 440 | 26.470 | 53.529 | 10.830 | 1.00 | 50.00 | O |
| ATOM | 9360 | OD2 | ASP | F | 440 | 26.016 | 54.289 | 8.831 | 1.00 | 50.00 | O |
| ATOM | 9361 | N | TYR | F | 441 | 30.299 | 50.688 | 9.957 | 1.00 | 50.00 | N |
| ATOM | 9362 | CA | TYR | F | 441 | 30.986 | 49.426 | 9.681 | 1.00 | 50.00 | C |
| ATOM | 9363 | C | TYR | F | 441 | 30.025 | 48.281 | 9.376 | 1.00 | 50.00 | C |
| ATOM | 9364 | O | TYR | F | 441 | 28.874 | 48.286 | 9.811 | 1.00 | 50.00 | O |
| ATOM | 9365 | CB | TYR | F | 441 | 31.885 | 49.181 | 10.895 | 1.00 | 50.00 | C |
| ATOM | 9366 | CG | TYR | F | 441 | 32.935 | 48.117 | 10.662 | 1.00 | 50.00 | C |
| ATOM | 9367 | CD1 | TYR | F | 441 | 33.923 | 48.311 | 9.764 | 1.00 | 50.00 | C |
| ATOM | 9368 | CD2 | TYR | F | 441 | 32.698 | 46.878 | 11.138 | 1.00 | 50.00 | C |
| ATOM | 9369 | CE1 | TYR | F | 441 | 34.603 | 47.260 | 9.265 | 1.00 | 50.00 | C |
| ATOM | 9370 | CE2 | TYR | F | 441 | 33.386 | 45.826 | 10.649 | 1.00 | 50.00 | C |
| ATOM | 9371 | CZ | TYR | F | 441 | 34.305 | 46.014 | 9.682 | 1.00 | 50.00 | C |
| ATOM | 9372 | OH | TYR | F | 441 | 34.925 | 44.954 | 9.076 | 1.00 | 50.00 | O |
| ATOM | 9373 | N | VAL | F | 442 | 30.502 | 47.277 | 8.658 | 1.00 | 50.00 | N |
| ATOM | 9374 | CA | VAL | F | 442 | 29.752 | 46.063 | 8.326 | 1.00 | 50.00 | C |
| ATOM | 9375 | C | VAL | F | 442 | 29.654 | 45.087 | 9.495 | 1.00 | 50.00 | C |
| ATOM | 9376 | O | VAL | F | 442 | 28.529 | 44.831 | 9.920 | 1.00 | 50.00 | O |
| ATOM | 9377 | CB | VAL | F | 442 | 30.220 | 45.465 | 6.995 | 1.00 | 50.00 | C |
| ATOM | 9378 | CG1 | VAL | F | 442 | 29.677 | 44.070 | 6.686 | 1.00 | 50.00 | C |
| ATOM | 9379 | CG2 | VAL | F | 442 | 29.864 | 46.344 | 5.797 | 1.00 | 50.00 | C |
| ATOM | 9380 | N | SER | F | 443 | 30.729 | 44.465 | 9.950 | 1.00 | 50.00 | N |
| ATOM | 9381 | CA | SER | F | 443 | 30.756 | 43.431 | 10.986 | 1.00 | 50.00 | C |
| ATOM | 9382 | C | SER | F | 443 | 29.939 | 42.197 | 10.626 | 1.00 | 50.00 | C |
| ATOM | 9383 | O | SER | F | 443 | 28.800 | 42.048 | 11.070 | 1.00 | 50.00 | O |
| ATOM | 9384 | CB | SER | F | 443 | 30.495 | 43.878 | 12.425 | 1.00 | 50.00 | C |
| ATOM | 9385 | OG | SER | F | 443 | 31.600 | 44.539 | 13.023 | 1.00 | 50.00 | O |
| ATOM | 9386 | N | ASN | F | 444 | 30.518 | 41.329 | 9.814 | 1.00 | 50.00 | N |
| ATOM | 9387 | CA | ASN | F | 444 | 29.902 | 40.060 | 9.426 | 1.00 | 50.00 | C |
| ATOM | 9388 | C | ASN | F | 444 | 30.795 | 38.898 | 9.840 | 1.00 | 50.00 | C |
| ATOM | 9389 | O | ASN | F | 444 | 31.880 | 39.066 | 10.397 | 1.00 | 50.00 | O |
| ATOM | 9390 | CB | ASN | F | 444 | 29.548 | 40.108 | 7.937 | 1.00 | 50.00 | C |
| ATOM | 9391 | CG | ASN | F | 444 | 28.370 | 40.952 | 7.468 | 1.00 | 50.00 | C |
| ATOM | 9392 | OD1 | ASN | F | 444 | 28.291 | 41.332 | 6.301 | 1.00 | 50.00 | O |
| ATOM | 9393 | ND2 | ASN | F | 444 | 27.404 | 41.249 | 8.317 | 1.00 | 50.00 | N |
| ATOM | 9394 | N | LYS | F | 445 | 30.316 | 37.704 | 9.533 | 1.00 | 50.00 | N |
| ATOM | 9395 | CA | LYS | F | 445 | 31.089 | 36.466 | 9.418 | 1.00 | 50.00 | C |
| ATOM | 9396 | C | LYS | F | 445 | 32.583 | 36.624 | 9.150 | 1.00 | 50.00 | C |
| ATOM | 9397 | O | LYS | F | 445 | 33.351 | 36.562 | 10.110 | 1.00 | 50.00 | O |
| ATOM | 9398 | CB | LYS | F | 445 | 30.357 | 35.542 | 8.442 | 1.00 | 50.00 | C |
| ATOM | 9399 | CG | LYS | F | 445 | 29.064 | 34.886 | 8.928 | 1.00 | 50.00 | C |
| ATOM | 9400 | CD | LYS | F | 445 | 28.649 | 33.628 | 8.165 | 1.00 | 50.00 | C |
| ATOM | 9401 | CE | LYS | F | 445 | 27.485 | 32.882 | 8.815 | 1.00 | 50.00 | C |
| ATOM | 9402 | NZ | LYS | F | 445 | 26.745 | 32.003 | 7.897 | 1.00 | 50.00 | N |
| ATOM | 9403 | N | GLY | F | 446 | 33.018 | 36.898 | 7.931 | 1.00 | 50.00 | N |
| ATOM | 9404 | CA | GLY | F | 446 | 34.436 | 36.991 | 7.584 | 1.00 | 50.00 | C |
| ATOM | 9405 | C | GLY | F | 446 | 35.359 | 37.933 | 8.347 | 1.00 | 50.00 | C |
| ATOM | 9406 | O | GLY | F | 446 | 36.486 | 37.542 | 8.651 | 1.00 | 50.00 | O |
| ATOM | 9407 | N | VAL | F | 447 | 34.901 | 39.125 | 8.692 | 1.00 | 50.00 | N |
| ATOM | 9408 | CA | VAL | F | 447 | 35.612 | 40.075 | 9.551 | 1.00 | 50.00 | C |
| ATOM | 9409 | C | VAL | F | 447 | 35.843 | 39.521 | 10.954 | 1.00 | 50.00 | C |
| ATOM | 9410 | O | VAL | F | 447 | 36.984 | 39.312 | 11.362 | 1.00 | 50.00 | O |
| ATOM | 9411 | CB | VAL | F | 447 | 34.902 | 41.432 | 9.516 | 1.00 | 50.00 | C |
| ATOM | 9412 | CG1 | VAL | F | 447 | 35.416 | 42.435 | 10.550 | 1.00 | 50.00 | C |
| ATOM | 9413 | CG2 | VAL | F | 447 | 34.890 | 42.087 | 8.134 | 1.00 | 50.00 | C |
| ATOM | 9414 | N | ASP | F | 448 | 34.758 | 39.366 | 11.695 | 1.00 | 50.00 | N |
| ATOM | 9415 | CA | ASP | F | 448 | 34.732 | 39.103 | 13.133 | 1.00 | 50.00 | C |
| ATOM | 9416 | C | ASP | F | 448 | 33.798 | 37.921 | 13.356 | 1.00 | 50.00 | C |
| ATOM | 9417 | O | ASP | F | 448 | 32.763 | 37.764 | 12.708 | 1.00 | 50.00 | O |
| ATOM | 9418 | CB | ASP | F | 448 | 34.296 | 40.337 | 13.926 | 1.00 | 50.00 | C |
| ATOM | 9419 | CG | ASP | F | 448 | 32.931 | 40.922 | 13.592 | 1.00 | 50.00 | C |
| ATOM | 9420 | OD1 | ASP | F | 448 | 32.812 | 41.606 | 12.553 | 1.00 | 50.00 | O |
| ATOM | 9421 | OD2 | ASP | F | 448 | 31.960 | 40.656 | 14.332 | 1.00 | 50.00 | O |
| ATOM | 9422 | N | THR | F | 449 | 34.148 | 37.100 | 14.330 | 1.00 | 50.00 | N |
| ATOM | 9423 | CA | THR | F | 449 | 33.366 | 35.921 | 14.705 | 1.00 | 50.00 | C |
| ATOM | 9424 | C | THR | F | 449 | 32.428 | 36.122 | 15.891 | 1.00 | 50.00 | C |
| ATOM | 9425 | O | THR | F | 449 | 31.482 | 35.342 | 15.989 | 1.00 | 50.00 | O |
| ATOM | 9426 | CB | THR | F | 449 | 34.366 | 34.768 | 14.813 | 1.00 | 50.00 | C |
| ATOM | 9427 | OG1 | THR | F | 449 | 34.905 | 34.483 | 13.530 | 1.00 | 50.00 | O |
| ATOM | 9428 | CG2 | THR | F | 449 | 33.882 | 33.442 | 15.394 | 1.00 | 50.00 | C |
| ATOM | 9429 | N | VAL | F | 450 | 32.639 | 37.070 | 16.790 | 1.00 | 50.00 | N |
| ATOM | 9430 | CA | VAL | F | 450 | 31.882 | 37.210 | 18.035 | 1.00 | 50.00 | C |
| ATOM | 9431 | C | VAL | F | 450 | 31.873 | 38.698 | 18.375 | 1.00 | 50.00 | C |
| ATOM | 9432 | O | VAL | F | 450 | 32.751 | 39.184 | 19.088 | 1.00 | 50.00 | O |
| ATOM | 9433 | CB | VAL | F | 450 | 32.370 | 36.278 | 19.150 | 1.00 | 50.00 | C |
| ATOM | 9434 | CG1 | VAL | F | 450 | 32.071 | 34.787 | 18.996 | 1.00 | 50.00 | C |
| ATOM | 9435 | CG2 | VAL | F | 450 | 33.874 | 36.326 | 19.415 | 1.00 | 50.00 | C |
| ATOM | 9436 | N | SER | F | 451 | 30.902 | 39.465 | 17.904 | 1.00 | 50.00 | N |
| ATOM | 9437 | CA | SER | F | 451 | 30.726 | 40.875 | 18.258 | 1.00 | 50.00 | C |
| ATOM | 9438 | C | SER | F | 451 | 30.310 | 41.114 | 19.706 | 1.00 | 50.00 | C |
| ATOM | 9439 | O | SER | F | 451 | 29.703 | 40.260 | 20.352 | 1.00 | 50.00 | O |
| ATOM | 9440 | CB | SER | F | 451 | 29.697 | 41.528 | 17.335 | 1.00 | 50.00 | C |
| ATOM | 9441 | OG | SER | F | 451 | 30.161 | 41.658 | 15.999 | 1.00 | 50.00 | O |
| ATOM | 9442 | N | VAL | F | 452 | 30.643 | 42.288 | 20.217 | 1.00 | 50.00 | N |
| ATOM | 9443 | CA | VAL | F | 452 | 30.258 | 42.771 | 21.543 | 1.00 | 50.00 | C |
| ATOM | 9444 | C | VAL | F | 452 | 30.023 | 44.264 | 21.336 | 1.00 | 50.00 | C |
| ATOM | 9445 | O | VAL | F | 452 | 30.968 | 45.005 | 21.070 | 1.00 | 50.00 | O |
| ATOM | 9446 | CB | VAL | F | 452 | 31.274 | 42.448 | 22.644 | 1.00 | 50.00 | C |
| ATOM | 9447 | CG1 | VAL | F | 452 | 31.016 | 43.145 | 23.980 | 1.00 | 50.00 | C |
| ATOM | 9448 | CG2 | VAL | F | 452 | 31.407 | 40.956 | 22.947 | 1.00 | 50.00 | C |
| ATOM | 9449 | N | GLY | F | 453 | 28.790 | 44.718 | 21.492 | 1.00 | 50.00 | N |
| ATOM | 9450 | CA | GLY | F | 453 | 28.440 | 46.131 | 21.353 | 1.00 | 50.00 | C |
| ATOM | 9451 | C | GLY | F | 453 | 27.900 | 46.774 | 22.623 | 1.00 | 50.00 | C |
| ATOM | 9452 | O | GLY | F | 453 | 26.723 | 47.133 | 22.633 | 1.00 | 50.00 | O |
| ATOM | 9453 | N | ASN | F | 454 | 28.735 | 46.946 | 23.637 | 1.00 | 50.00 | N |
| ATOM | 9454 | CA | ASN | F | 454 | 28.418 | 47.640 | 24.887 | 1.00 | 50.00 | C |
| ATOM | 9455 | C | ASN | F | 454 | 27.084 | 47.244 | 25.512 | 1.00 | 50.00 | C |
| ATOM | 9456 | O | ASN | F | 454 | 26.123 | 47.981 | 25.292 | 1.00 | 50.00 | O |
| ATOM | 9457 | CB | ASN | F | 454 | 28.567 | 49.156 | 24.733 | 1.00 | 50.00 | C |
| ATOM | 9458 | CG | ASN | F | 454 | 29.941 | 49.786 | 24.550 | 1.00 | 50.00 | C |
| ATOM | 9459 | OD1 | ASN | F | 454 | 30.172 | 50.552 | 23.615 | 1.00 | 50.00 | O |
| ATOM | 9460 | ND2 | ASN | F | 454 | 30.867 | 49.553 | 25.461 | 1.00 | 50.00 | N |
| ATOM | 9461 | N | THR | F | 455 | 27.001 | 46.112 | 26.195 | 1.00 | 50.00 | N |
| ATOM | 9462 | CA | THR | F | 455 | 25.786 | 45.491 | 26.731 | 1.00 | 50.00 | C |
| ATOM | 9463 | C | THR | F | 455 | 25.625 | 44.056 | 26.234 | 1.00 | 50.00 | C |
| ATOM | 9464 | O | THR | F | 455 | 25.383 | 43.189 | 27.072 | 1.00 | 50.00 | O |
| ATOM | 9465 | CB | THR | F | 455 | 24.483 | 46.294 | 26.794 | 1.00 | 50.00 | C |
| ATOM | 9466 | OG1 | THR | F | 455 | 24.721 | 47.534 | 27.445 | 1.00 | 50.00 | O |
| ATOM | 9467 | CG2 | THR | F | 455 | 23.245 | 45.776 | 27.524 | 1.00 | 50.00 | C |
| ATOM | 9468 | N | LEU | F | 456 | 25.674 | 43.766 | 24.942 | 1.00 | 50.00 | N |
| ATOM | 9469 | CA | LEU | F | 456 | 25.258 | 42.489 | 24.355 | 1.00 | 50.00 | C |
| ATOM | 9470 | C | LEU | F | 456 | 26.445 | 41.795 | 23.696 | 1.00 | 50.00 | C |
| ATOM | 9471 | O | LEU | F | 456 | 27.358 | 42.438 | 23.179 | 1.00 | 50.00 | O |
| ATOM | 9472 | CB | LEU | F | 456 | 24.138 | 42.642 | 23.323 | 1.00 | 50.00 | C |
| ATOM | 9473 | CG | LEU | F | 456 | 22.718 | 42.966 | 23.791 | 1.00 | 50.00 | C |
| ATOM | 9474 | CD1 | LEU | F | 456 | 22.601 | 44.355 | 24.415 | 1.00 | 50.00 | C |
| ATOM | 9475 | CD2 | LEU | F | 456 | 21.681 | 42.910 | 22.672 | 1.00 | 50.00 | C |
| ATOM | 9476 | N | TYR | F | 457 | 26.387 | 40.474 | 23.655 | 1.00 | 50.00 | N |
| ATOM | 9477 | CA | TYR | F | 457 | 27.295 | 39.631 | 22.875 | 1.00 | 50.00 | C |
| ATOM | 9478 | C | TYR | F | 457 | 26.471 | 39.107 | 21.703 | 1.00 | 50.00 | C |
| ATOM | 9479 | O | TYR | F | 457 | 25.427 | 38.479 | 21.879 | 1.00 | 50.00 | O |
| ATOM | 9480 | CB | TYR | F | 457 | 28.024 | 38.653 | 23.803 | 1.00 | 50.00 | C |
| ATOM | 9481 | CG | TYR | F | 457 | 27.847 | 37.151 | 23.712 | 1.00 | 50.00 | C |
| ATOM | 9482 | CD1 | TYR | F | 457 | 26.606 | 36.620 | 23.701 | 1.00 | 50.00 | C |
| ATOM | 9483 | CD2 | TYR | F | 457 | 28.908 | 36.330 | 23.567 | 1.00 | 50.00 | C |
| ATOM | 9484 | CE1 | TYR | F | 457 | 26.414 | 35.324 | 23.388 | 1.00 | 50.00 | C |
| ATOM | 9485 | CE2 | TYR | F | 457 | 28.729 | 35.015 | 23.323 | 1.00 | 50.00 | C |
| ATOM | 9486 | CZ | TYR | F | 457 | 27.481 | 34.517 | 23.216 | 1.00 | 50.00 | C |
| ATOM | 9487 | OH | TYR | F | 457 | 27.271 | 33.215 | 22.846 | 1.00 | 50.00 | O |
| ATOM | 9488 | N | TYR | F | 458 | 26.913 | 39.349 | 20.481 | 1.00 | 50.00 | N |
| ATOM | 9489 | CA | TYR | F | 458 | 26.422 | 38.643 | 19.298 | 1.00 | 50.00 | C |
| ATOM | 9490 | C | TYR | F | 458 | 27.453 | 37.626 | 18.822 | 1.00 | 50.00 | C |
| ATOM | 9491 | O | TYR | F | 458 | 28.646 | 37.923 | 18.773 | 1.00 | 50.00 | O |
| ATOM | 9492 | CB | TYR | F | 458 | 26.011 | 39.697 | 18.268 | 1.00 | 50.00 | C |
| ATOM | 9493 | CG | TYR | F | 458 | 25.278 | 39.117 | 17.079 | 1.00 | 50.00 | C |
| ATOM | 9494 | CD1 | TYR | F | 458 | 24.061 | 38.578 | 17.298 | 1.00 | 50.00 | C |
| ATOM | 9495 | CD2 | TYR | F | 458 | 25.934 | 38.858 | 15.929 | 1.00 | 50.00 | C |
| ATOM | 9496 | CE1 | TYR | F | 458 | 23.543 | 37.706 | 16.413 | 1.00 | 50.00 | C |
| ATOM | 9497 | CE2 | TYR | F | 458 | 25.410 | 37.999 | 15.031 | 1.00 | 50.00 | C |
| ATOM | 9498 | CZ | TYR | F | 458 | 24.229 | 37.407 | 15.293 | 1.00 | 50.00 | C |
| ATOM | 9499 | OH | TYR | F | 458 | 23.935 | 36.205 | 14.713 | 1.00 | 50.00 | O |
| ATOM | 9500 | N | VAL | F | 459 | 27.010 | 36.441 | 18.433 | 1.00 | 50.00 | N |
| ATOM | 9501 | CA | VAL | F | 459 | 27.854 | 35.417 | 17.814 | 1.00 | 50.00 | C |
| ATOM | 9502 | C | VAL | F | 459 | 27.617 | 35.480 | 16.310 | 1.00 | 50.00 | C |
| ATOM | 9503 | O | VAL | F | 459 | 26.552 | 35.090 | 15.830 | 1.00 | 50.00 | O |
| ATOM | 9504 | CB | VAL | F | 459 | 27.597 | 34.037 | 18.423 | 1.00 | 50.00 | C |
| ATOM | 9505 | CG1 | VAL | F | 459 | 28.283 | 32.877 | 17.704 | 1.00 | 50.00 | C |
| ATOM | 9506 | CG2 | VAL | F | 459 | 28.023 | 33.941 | 19.888 | 1.00 | 50.00 | C |
| ATOM | 9507 | N | ASN | F | 460 | 28.629 | 35.898 | 15.568 | 1.00 | 50.00 | N |
| ATOM | 9508 | CA | ASN | F | 460 | 28.661 | 35.793 | 14.109 | 1.00 | 50.00 | C |
| ATOM | 9509 | C | ASN | F | 460 | 29.138 | 34.460 | 13.532 | 1.00 | 50.00 | C |
| ATOM | 9510 | O | ASN | F | 460 | 29.083 | 34.435 | 12.304 | 1.00 | 50.00 | O |
| ATOM | 9511 | CB | ASN | F | 460 | 29.378 | 37.003 | 13.501 | 1.00 | 50.00 | C |
| ATOM | 9512 | CG | ASN | F | 460 | 28.864 | 38.428 | 13.671 | 1.00 | 50.00 | C |
| ATOM | 9513 | OD1 | ASN | F | 460 | 29.410 | 39.206 | 14.452 | 1.00 | 50.00 | O |
| ATOM | 9514 | ND2 | ASN | F | 460 | 27.867 | 38.870 | 12.926 | 1.00 | 50.00 | N |
| ATOM | 9515 | N | LYS | F | 461 | 29.507 | 33.367 | 14.191 | 1.00 | 50.00 | N |
| ATOM | 9516 | CA | LYS | F | 461 | 29.802 | 32.073 | 13.562 | 1.00 | 50.00 | C |
| ATOM | 9517 | C | LYS | F | 461 | 29.524 | 30.771 | 14.307 | 1.00 | 50.00 | C |
| ATOM | 9518 | O | LYS | F | 461 | 30.025 | 30.592 | 15.416 | 1.00 | 50.00 | O |
| ATOM | 9519 | CB | LYS | F | 461 | 31.168 | 31.851 | 12.918 | 1.00 | 50.00 | C |
| ATOM | 9520 | CG | LYS | F | 461 | 31.393 | 32.744 | 11.702 | 1.00 | 50.00 | C |
| ATOM | 9521 | CD | LYS | F | 461 | 32.743 | 32.428 | 11.067 | 1.00 | 50.00 | C |
| ATOM | 9522 | CE | LYS | F | 461 | 32.878 | 33.387 | 9.890 | 1.00 | 50.00 | C |
| ATOM | 9523 | NZ | LYS | F | 461 | 34.254 | 33.470 | 9.383 | 1.00 | 50.00 | N |
| ATOM | 9524 | N | GLN | F | 462 | 28.780 | 29.866 | 13.684 | 1.00 | 50.00 | N |
| ATOM | 9525 | CA | GLN | F | 462 | 28.241 | 28.641 | 14.278 | 1.00 | 50.00 | C |
| ATOM | 9526 | C | GLN | F | 462 | 27.568 | 27.756 | 13.232 | 1.00 | 50.00 | C |
| ATOM | 9527 | O | GLN | F | 462 | 26.925 | 28.219 | 12.290 | 1.00 | 50.00 | O |
| ATOM | 9528 | CB | GLN | F | 462 | 27.184 | 28.825 | 15.369 | 1.00 | 50.00 | C |
| ATOM | 9529 | CG | GLN | F | 462 | 27.491 | 29.467 | 16.719 | 1.00 | 50.00 | C |
| ATOM | 9530 | CD | GLN | F | 462 | 28.650 | 28.851 | 17.492 | 1.00 | 50.00 | C |
| ATOM | 9531 | OE1 | GLN | F | 462 | 28.989 | 27.677 | 17.353 | 1.00 | 50.00 | O |
| ATOM | 9532 | NE2 | GLN | F | 462 | 29.314 | 29.641 | 18.314 | 1.00 | 50.00 | N |
| ATOM | 9533 | N | GLU | F | 463 | 27.637 | 26.459 | 13.487 | 1.00 | 50.00 | N |
| ATOM | 9534 | CA | GLU | F | 463 | 27.000 | 25.395 | 12.710 | 1.00 | 50.00 | C |
| ATOM | 9535 | C | GLU | F | 463 | 26.450 | 24.305 | 13.624 | 1.00 | 50.00 | C |
| ATOM | 9536 | O | GLU | F | 463 | 26.741 | 24.215 | 14.817 | 1.00 | 50.00 | O |
| ATOM | 9537 | CB | GLU | F | 463 | 28.024 | 24.856 | 11.708 | 1.00 | 50.00 | C |
| ATOM | 9538 | CG | GLU | F | 463 | 28.047 | 25.590 | 10.367 | 1.00 | 50.00 | C |
| ATOM | 9539 | CD | GLU | F | 463 | 29.326 | 25.491 | 9.550 | 1.00 | 50.00 | C |
| ATOM | 9540 | OE1 | GLU | F | 463 | 30.392 | 25.103 | 10.077 | 1.00 | 50.00 | O |
| ATOM | 9541 | OE2 | GLU | F | 463 | 29.277 | 25.838 | 8.350 | 1.00 | 50.00 | O |
| ATOM | 9542 | N | GLY | F | 464 | 25.638 | 23.444 | 13.032 | 1.00 | 50.00 | N |
| ATOM | 9543 | CA | GLY | F | 464 | 25.117 | 22.234 | 13.667 | 1.00 | 50.00 | C |
| ATOM | 9544 | C | GLY | F | 464 | 24.702 | 21.225 | 12.604 | 1.00 | 50.00 | C |
| ATOM | 9545 | O | GLY | F | 464 | 24.118 | 21.578 | 11.580 | 1.00 | 50.00 | O |
| ATOM | 9546 | N | LYS | F | 465 | 25.015 | 19.965 | 12.858 | 1.00 | 50.00 | N |
| ATOM | 9547 | CA | LYS | F | 465 | 24.762 | 18.858 | 11.935 | 1.00 | 50.00 | C |
| ATOM | 9548 | C | LYS | F | 465 | 24.161 | 17.691 | 12.711 | 1.00 | 50.00 | C |
| ATOM | 9549 | O | LYS | F | 465 | 24.552 | 17.397 | 13.840 | 1.00 | 50.00 | O |
| ATOM | 9550 | CB | LYS | F | 465 | 26.028 | 18.433 | 11.190 | 1.00 | 50.00 | C |
| ATOM | 9551 | CG | LYS | F | 465 | 26.607 | 19.482 | 10.242 | 1.00 | 50.00 | C |
| ATOM | 9552 | CD | LYS | F | 465 | 27.858 | 19.019 | 9.498 | 1.00 | 50.00 | C |
| ATOM | 9553 | CE | LYS | F | 465 | 28.465 | 20.138 | 8.655 | 1.00 | 50.00 | C |
| ATOM | 9554 | NZ | LYS | F | 465 | 29.657 | 19.673 | 7.931 | 1.00 | 50.00 | N |
| ATOM | 9555 | N | SER | F | 466 | 23.198 | 17.025 | 12.095 | 1.00 | 50.00 | N |
| ATOM | 9556 | CA | SER | F | 466 | 22.438 | 15.931 | 12.701 | 1.00 | 50.00 | C |
| ATOM | 9557 | C | SER | F | 466 | 21.783 | 15.078 | 11.622 | 1.00 | 50.00 | C |
| ATOM | 9558 | O | SER | F | 466 | 21.418 | 15.536 | 10.539 | 1.00 | 50.00 | O |
| ATOM | 9559 | CB | SER | F | 466 | 21.365 | 16.421 | 13.675 | 1.00 | 50.00 | C |
| ATOM | 9560 | OG | SER | F | 466 | 21.930 | 16.900 | 14.886 | 1.00 | 50.00 | O |
| ATOM | 9561 | N | LEU | F | 467 | 21.649 | 13.803 | 11.949 | 1.00 | 50.00 | N |
| ATOM | 9562 | CA | LEU | F | 467 | 21.096 | 12.770 | 11.073 | 1.00 | 50.00 | C |
| ATOM | 9563 | C | LEU | F | 467 | 19.932 | 12.140 | 11.832 | 1.00 | 50.00 | C |
| ATOM | 9564 | O | LEU | F | 467 | 20.078 | 11.671 | 12.961 | 1.00 | 50.00 | O |
| ATOM | 9565 | CB | LEU | F | 467 | 22.178 | 11.759 | 10.686 | 1.00 | 50.00 | C |
| ATOM | 9566 | CG | LEU | F | 467 | 23.283 | 12.158 | 9.705 | 1.00 | 50.00 | C |
| ATOM | 9567 | CD1 | LEU | F | 467 | 24.301 | 13.191 | 10.188 | 1.00 | 50.00 | C |
| ATOM | 9568 | CD2 | LEU | F | 467 | 24.079 | 10.929 | 9.269 | 1.00 | 50.00 | C |
| ATOM | 9569 | N | TYR | F | 468 | 18.765 | 12.131 | 11.210 | 1.00 | 50.00 | N |
| ATOM | 9570 | CA | TYR | F | 468 | 17.539 | 11.514 | 11.719 | 1.00 | 50.00 | C |
| ATOM | 9571 | C | TYR | F | 468 | 17.378 | 10.201 | 10.964 | 1.00 | 50.00 | C |
| ATOM | 9572 | O | TYR | F | 468 | 17.170 | 10.200 | 9.751 | 1.00 | 50.00 | O |
| ATOM | 9573 | CB | TYR | F | 468 | 16.339 | 12.443 | 11.517 | 1.00 | 50.00 | C |
| ATOM | 9574 | CG | TYR | F | 468 | 15.026 | 12.068 | 12.172 | 1.00 | 50.00 | C |
| ATOM | 9575 | CD1 | TYR | F | 468 | 14.441 | 12.952 | 13.008 | 1.00 | 50.00 | C |
| ATOM | 9576 | CD2 | TYR | F | 468 | 14.460 | 10.854 | 12.009 | 1.00 | 50.00 | C |
| ATOM | 9577 | CE1 | TYR | F | 468 | 13.372 | 12.590 | 13.746 | 1.00 | 50.00 | C |
| ATOM | 9578 | CE2 | TYR | F | 468 | 13.445 | 10.460 | 12.805 | 1.00 | 50.00 | C |
| ATOM | 9579 | CZ | TYR | F | 468 | 12.922 | 11.320 | 13.700 | 1.00 | 50.00 | C |
| ATOM | 9580 | OH | TYR | F | 468 | 12.030 | 10.878 | 14.641 | 1.00 | 50.00 | O |
| ATOM | 9581 | N | VAL | F | 469 | 17.461 | 9.088 | 11.673 | 1.00 | 50.00 | N |
| ATOM | 9582 | CA | VAL | F | 469 | 17.485 | 7.753 | 11.074 | 1.00 | 50.00 | C |
| ATOM | 9583 | C | VAL | F | 469 | 16.813 | 6.786 | 12.044 | 1.00 | 50.00 | C |
| ATOM | 9584 | O | VAL | F | 469 | 16.844 | 6.964 | 13.261 | 1.00 | 50.00 | O |
| ATOM | 9585 | CB | VAL | F | 469 | 18.858 | 7.302 | 10.563 | 1.00 | 50.00 | C |
| ATOM | 9586 | CG1 | VAL | F | 469 | 19.799 | 8.377 | 10.019 | 1.00 | 50.00 | C |
| ATOM | 9587 | CG2 | VAL | F | 469 | 19.724 | 6.474 | 11.513 | 1.00 | 50.00 | C |
| ATOM | 9588 | N | LYS | F | 470 | 16.316 | 5.683 | 11.510 | 1.00 | 50.00 | N |
| ATOM | 9589 | CA | LYS | F | 470 | 15.966 | 4.502 | 12.299 | 1.00 | 50.00 | C |
| ATOM | 9590 | C | LYS | F | 470 | 16.820 | 3.278 | 11.989 | 1.00 | 50.00 | C |
| ATOM | 9591 | O | LYS | F | 470 | 17.551 | 3.216 | 11.001 | 1.00 | 50.00 | O |
| ATOM | 9592 | CB | LYS | F | 470 | 14.465 | 4.273 | 12.125 | 1.00 | 50.00 | C |
| ATOM | 9593 | CG | LYS | F | 470 | 13.928 | 3.558 | 13.363 | 1.00 | 50.00 | C |
| ATOM | 9594 | CD | LYS | F | 470 | 12.407 | 3.542 | 13.311 | 1.00 | 50.00 | C |
| ATOM | 9595 | CE | LYS | F | 470 | 11.820 | 2.655 | 14.403 | 1.00 | 50.00 | C |
| ATOM | 9596 | NZ | LYS | F | 470 | 10.504 | 2.413 | 13.800 | 1.00 | 50.00 | N |
| ATOM | 9597 | N | GLY | F | 471 | 16.728 | 2.287 | 12.860 | 1.00 | 50.00 | N |
| ATOM | 9598 | CA | GLY | F | 471 | 17.147 | 0.914 | 12.584 | 1.00 | 50.00 | C |
| ATOM | 9599 | C | GLY | F | 471 | 16.071 | −0.059 | 13.047 | 1.00 | 50.00 | C |
| ATOM | 9600 | O | GLY | F | 471 | 15.861 | −0.179 | 14.254 | 1.00 | 50.00 | O |
| ATOM | 9601 | N | GLU | F | 472 | 15.448 | −0.784 | 12.132 | 1.00 | 50.00 | N |
| ATOM | 9602 | CA | GLU | F | 472 | 14.534 | −1.882 | 12.454 | 1.00 | 50.00 | C |
| ATOM | 9603 | C | GLU | F | 472 | 15.186 | −2.987 | 13.279 | 1.00 | 50.00 | C |
| ATOM | 9604 | O | GLU | F | 472 | 16.273 | −3.426 | 12.904 | 1.00 | 50.00 | O |
| ATOM | 9605 | CB | GLU | F | 472 | 13.822 | −2.462 | 11.229 | 1.00 | 50.00 | C |
| ATOM | 9606 | CG | GLU | F | 472 | 13.619 | −3.959 | 10.990 | 1.00 | 50.00 | C |
| ATOM | 9607 | CD | GLU | F | 472 | 14.847 | −4.661 | 10.428 | 1.00 | 50.00 | C |
| ATOM | 9608 | OE1 | GLU | F | 472 | 15.478 | −5.474 | 11.139 | 1.00 | 50.00 | O |
| ATOM | 9609 | OE2 | GLU | F | 472 | 15.199 | −4.407 | 9.257 | 1.00 | 50.00 | O |
| ATOM | 9610 | N | PRO | F | 473 | 14.514 | −3.535 | 14.280 | 1.00 | 50.00 | N |
| ATOM | 9611 | CA | PRO | F | 473 | 14.884 | −4.822 | 14.867 | 1.00 | 50.00 | C |
| ATOM | 9612 | C | PRO | F | 473 | 13.815 | −5.893 | 14.672 | 1.00 | 50.00 | C |
| ATOM | 9613 | O | PRO | F | 473 | 12.791 | −5.882 | 15.355 | 1.00 | 50.00 | O |
| ATOM | 9614 | CB | PRO | F | 473 | 15.099 | −4.366 | 16.309 | 1.00 | 50.00 | C |
| ATOM | 9615 | CG | PRO | F | 473 | 14.057 | −3.281 | 16.571 | 1.00 | 50.00 | C |
| ATOM | 9616 | CD | PRO | F | 473 | 13.703 | −2.729 | 15.193 | 1.00 | 50.00 | C |
| ATOM | 9617 | N | ILE | F | 474 | 14.049 | −6.841 | 13.778 | 1.00 | 50.00 | N |
| ATOM | 9618 | CA | ILE | F | 474 | 13.199 | −8.022 | 13.606 | 1.00 | 50.00 | C |
| ATOM | 9619 | C | ILE | F | 474 | 13.425 | −9.027 | 14.731 | 1.00 | 50.00 | C |
| ATOM | 9620 | O | ILE | F | 474 | 14.569 | −9.316 | 15.081 | 1.00 | 50.00 | O |
| ATOM | 9621 | CB | ILE | F | 474 | 13.316 | −8.672 | 12.224 | 1.00 | 50.00 | C |
| ATOM | 9622 | CG1 | ILE | F | 474 | 14.672 | −9.285 | 11.872 | 1.00 | 50.00 | C |
| ATOM | 9623 | CG2 | ILE | F | 474 | 12.826 | −7.750 | 11.109 | 1.00 | 50.00 | C |
| ATOM | 9624 | CD1 | ILE | F | 474 | 14.644 | −10.288 | 10.721 | 1.00 | 50.00 | C |
| ATOM | 9625 | N | ILE | F | 475 | 12.356 | −9.578 | 15.284 | 1.00 | 50.00 | N |
| ATOM | 9626 | CA | ILE | F | 475 | 12.421 | −10.636 | 16.293 | 1.00 | 50.00 | C |
| ATOM | 9627 | C | ILE | F | 475 | 11.896 | −11.894 | 15.609 | 1.00 | 50.00 | C |
| ATOM | 9628 | O | ILE | F | 475 | 10.717 | −11.966 | 15.261 | 1.00 | 50.00 | O |
| ATOM | 9629 | CB | ILE | F | 475 | 11.690 | −10.276 | 17.591 | 1.00 | 50.00 | C |
| ATOM | 9630 | CG1 | ILE | F | 475 | 12.233 | −9.024 | 18.285 | 1.00 | 50.00 | C |
| ATOM | 9631 | CG2 | ILE | F | 475 | 11.683 | −11.436 | 18.588 | 1.00 | 50.00 | C |
| ATOM | 9632 | CD1 | ILE | F | 475 | 11.324 | −8.404 | 19.345 | 1.00 | 50.00 | C |
| ATOM | 9633 | N | ASN | F | 476 | 12.749 | −12.897 | 15.475 | 1.00 | 50.00 | N |
| ATOM | 9634 | CA | ASN | F | 476 | 12.384 | −14.211 | 14.945 | 1.00 | 50.00 | C |
| ATOM | 9635 | C | ASN | F | 476 | 12.612 | −15.276 | 16.011 | 1.00 | 50.00 | C |
| ATOM | 9636 | O | ASN | F | 476 | 13.754 | −15.567 | 16.364 | 1.00 | 50.00 | O |
| ATOM | 9637 | CB | ASN | F | 476 | 13.169 | −14.536 | 13.673 | 1.00 | 50.00 | C |
| ATOM | 9638 | CG | ASN | F | 476 | 12.937 | −13.669 | 12.444 | 1.00 | 50.00 | C |
| ATOM | 9639 | OD1 | ASN | F | 476 | 13.877 | −13.275 | 11.755 | 1.00 | 50.00 | O |
| ATOM | 9640 | ND2 | ASN | F | 476 | 11.696 | −13.366 | 12.115 | 1.00 | 50.00 | N |
| ATOM | 9641 | N | PHE | F | 477 | 11.532 | −15.878 | 16.484 | 1.00 | 50.00 | N |
| ATOM | 9642 | CA | PHE | F | 477 | 11.574 | −17.081 | 17.315 | 1.00 | 50.00 | C |
| ATOM | 9643 | C | PHE | F | 477 | 11.137 | −18.273 | 16.470 | 1.00 | 50.00 | C |
| ATOM | 9644 | O | PHE | F | 477 | 10.051 | −18.284 | 15.892 | 1.00 | 50.00 | O |
| ATOM | 9645 | CB | PHE | F | 477 | 10.723 | −16.911 | 18.575 | 1.00 | 50.00 | C |
| ATOM | 9646 | CG | PHE | F | 477 | 10.840 | −18.037 | 19.581 | 1.00 | 50.00 | C |
| ATOM | 9647 | CD1 | PHE | F | 477 | 11.931 | −18.131 | 20.370 | 1.00 | 50.00 | C |
| ATOM | 9648 | CD2 | PHE | F | 477 | 9.899 | −19.002 | 19.638 | 1.00 | 50.00 | C |
| ATOM | 9649 | CE1 | PHE | F | 477 | 12.088 | −19.183 | 21.198 | 1.00 | 50.00 | C |
| ATOM | 9650 | CE2 | PHE | F | 477 | 10.050 | −20.056 | 20.465 | 1.00 | 50.00 | C |
| ATOM | 9651 | CZ | PHE | F | 477 | 11.146 | −20.147 | 21.244 | 1.00 | 50.00 | C |
| ATOM | 9652 | N | TYR | F | 478 | 11.981 | −19.291 | 16.462 | 1.00 | 50.00 | N |
| ATOM | 9653 | CA | TYR | F | 478 | 11.720 | −20.566 | 15.796 | 1.00 | 50.00 | C |
| ATOM | 9654 | C | TYR | F | 478 | 11.756 | −21.691 | 16.823 | 1.00 | 50.00 | C |
| ATOM | 9655 | O | TYR | F | 478 | 12.713 | −21.828 | 17.585 | 1.00 | 50.00 | O |
| ATOM | 9656 | CB | TYR | F | 478 | 12.748 | −20.743 | 14.677 | 1.00 | 50.00 | C |
| ATOM | 9657 | CG | TYR | F | 478 | 12.567 | −22.002 | 13.860 | 1.00 | 50.00 | C |
| ATOM | 9658 | CD1 | TYR | F | 478 | 11.546 | −22.033 | 12.979 | 1.00 | 50.00 | C |
| ATOM | 9659 | CD2 | TYR | F | 478 | 13.319 | −23.106 | 14.052 | 1.00 | 50.00 | C |
| ATOM | 9660 | CE1 | TYR | F | 478 | 11.298 | −23.159 | 12.284 | 1.00 | 50.00 | C |
| ATOM | 9661 | CE2 | TYR | F | 478 | 13.050 | −24.239 | 13.372 | 1.00 | 50.00 | C |
| ATOM | 9662 | CZ | TYR | F | 478 | 12.039 | −24.268 | 12.480 | 1.00 | 50.00 | C |
| ATOM | 9663 | OH | TYR | F | 478 | 11.790 | −25.421 | 11.783 | 1.00 | 50.00 | O |
| ATOM | 9664 | N | ASP | F | 479 | 10.747 | −22.544 | 16.761 | 1.00 | 50.00 | N |
| ATOM | 9665 | CA | ASP | F | 479 | 10.736 | −23.836 | 17.447 | 1.00 | 50.00 | C |
| ATOM | 9666 | C | ASP | F | 479 | 10.957 | −24.961 | 16.442 | 1.00 | 50.00 | C |
| ATOM | 9667 | O | ASP | F | 479 | 10.222 | −24.998 | 15.456 | 1.00 | 50.00 | O |
| ATOM | 9668 | CB | ASP | F | 479 | 9.469 | −24.035 | 18.281 | 1.00 | 50.00 | C |
| ATOM | 9669 | CG | ASP | F | 479 | 8.126 | −24.151 | 17.573 | 1.00 | 50.00 | C |
| ATOM | 9670 | OD1 | ASP | F | 479 | 7.583 | −23.117 | 17.129 | 1.00 | 50.00 | O |
| ATOM | 9671 | OD2 | ASP | F | 479 | 7.607 | −25.281 | 17.443 | 1.00 | 50.00 | O |
| ATOM | 9672 | N | PRO | F | 480 | 11.823 | −25.943 | 16.650 | 1.00 | 50.00 | N |
| ATOM | 9673 | CA | PRO | F | 480 | 11.714 | −27.233 | 15.970 | 1.00 | 50.00 | C |
| ATOM | 9674 | C | PRO | F | 480 | 10.555 | −28.025 | 16.564 | 1.00 | 50.00 | C |
| ATOM | 9675 | O | PRO | F | 480 | 10.618 | −28.587 | 17.657 | 1.00 | 50.00 | O |
| ATOM | 9676 | CB | PRO | F | 480 | 13.110 | −27.819 | 16.167 | 1.00 | 50.00 | C |
| ATOM | 9677 | CG | PRO | F | 480 | 13.574 | −27.296 | 17.524 | 1.00 | 50.00 | C |
| ATOM | 9678 | CD | PRO | F | 480 | 12.915 | −25.922 | 17.622 | 1.00 | 50.00 | C |
| ATOM | 9679 | N | LEU | F | 481 | 9.482 | −28.102 | 15.796 | 1.00 | 50.00 | N |
| ATOM | 9680 | CA | LEU | F | 481 | 8.677 | −29.315 | 15.658 | 1.00 | 50.00 | C |
| ATOM | 9681 | C | LEU | F | 481 | 9.551 | −30.472 | 15.182 | 1.00 | 50.00 | C |
| ATOM | 9682 | O | LEU | F | 481 | 10.016 | −30.481 | 14.042 | 1.00 | 50.00 | O |
| ATOM | 9683 | CB | LEU | F | 481 | 7.529 | −28.975 | 14.706 | 1.00 | 50.00 | C |
| ATOM | 9684 | CG | LEU | F | 481 | 6.285 | −28.293 | 15.279 | 1.00 | 50.00 | C |
| ATOM | 9685 | CD1 | LEU | F | 481 | 5.236 | −27.953 | 14.220 | 1.00 | 50.00 | C |
| ATOM | 9686 | CD2 | LEU | F | 481 | 5.633 | −29.065 | 16.425 | 1.00 | 50.00 | C |
| ATOM | 9687 | N | VAL | F | 482 | 9.864 | −31.366 | 16.106 | 1.00 | 50.00 | N |
| ATOM | 9688 | CA | VAL | F | 482 | 10.875 | −32.409 | 15.928 | 1.00 | 50.00 | C |
| ATOM | 9689 | C | VAL | F | 482 | 10.054 | −33.684 | 15.804 | 1.00 | 50.00 | C |
| ATOM | 9690 | O | VAL | F | 482 | 9.507 | −34.218 | 16.769 | 1.00 | 50.00 | O |
| ATOM | 9691 | CB | VAL | F | 482 | 11.925 | −32.571 | 17.031 | 1.00 | 50.00 | C |
| ATOM | 9692 | CG1 | VAL | F | 482 | 12.995 | −33.613 | 16.703 | 1.00 | 50.00 | C |
| ATOM | 9693 | CG2 | VAL | F | 482 | 12.683 | −31.287 | 17.360 | 1.00 | 50.00 | C |
| ATOM | 9694 | N | PHE | F | 483 | 10.075 | −34.205 | 14.591 | 1.00 | 50.00 | N |
| ATOM | 9695 | CA | PHE | F | 483 | 9.570 | −35.545 | 14.304 | 1.00 | 50.00 | C |
| ATOM | 9696 | C | PHE | F | 483 | 10.460 | −36.630 | 14.907 | 1.00 | 50.00 | C |
| ATOM | 9697 | O | PHE | F | 483 | 11.671 | −36.512 | 14.717 | 1.00 | 50.00 | O |
| ATOM | 9698 | CB | PHE | F | 483 | 9.454 | −35.612 | 12.780 | 1.00 | 50.00 | C |
| ATOM | 9699 | CG | PHE | F | 483 | 8.393 | −36.570 | 12.284 | 1.00 | 50.00 | C |
| ATOM | 9700 | CD1 | PHE | F | 483 | 7.223 | −36.148 | 11.761 | 1.00 | 50.00 | C |
| ATOM | 9701 | CD2 | PHE | F | 483 | 8.502 | −37.846 | 12.705 | 1.00 | 50.00 | C |
| ATOM | 9702 | CE1 | PHE | F | 483 | 6.140 | −36.950 | 11.806 | 1.00 | 50.00 | C |
| ATOM | 9703 | CE2 | PHE | F | 483 | 7.371 | −38.502 | 13.022 | 1.00 | 50.00 | C |
| ATOM | 9704 | CZ | PHE | F | 483 | 6.184 | −38.073 | 12.548 | 1.00 | 50.00 | C |
| ATOM | 9705 | N | PRO | F | 484 | 9.992 | −37.698 | 15.540 | 1.00 | 50.00 | N |
| ATOM | 9706 | CA | PRO | F | 484 | 10.793 | −38.892 | 15.820 | 1.00 | 50.00 | C |
| ATOM | 9707 | C | PRO | F | 484 | 11.181 | −39.748 | 14.616 | 1.00 | 50.00 | C |
| ATOM | 9708 | O | PRO | F | 484 | 10.752 | −40.892 | 14.468 | 1.00 | 50.00 | O |
| ATOM | 9709 | CB | PRO | F | 484 | 9.906 | −39.610 | 16.839 | 1.00 | 50.00 | C |
| ATOM | 9710 | CG | PRO | F | 484 | 8.483 | −39.290 | 16.391 | 1.00 | 50.00 | C |
| ATOM | 9711 | CD | PRO | F | 484 | 8.625 | −37.810 | 16.050 | 1.00 | 50.00 | C |
| ATOM | 9712 | N | SER | F | 485 | 12.012 | −39.206 | 13.739 | 1.00 | 50.00 | N |
| ATOM | 9713 | CA | SER | F | 485 | 12.342 | −39.773 | 12.431 | 1.00 | 50.00 | C |
| ATOM | 9714 | C | SER | F | 485 | 13.287 | −40.969 | 12.423 | 1.00 | 50.00 | C |
| ATOM | 9715 | O | SER | F | 485 | 13.057 | −41.890 | 11.640 | 1.00 | 50.00 | O |
| ATOM | 9716 | CB | SER | F | 485 | 12.865 | −38.649 | 11.536 | 1.00 | 50.00 | C |
| ATOM | 9717 | OG | SER | F | 485 | 11.793 | −37.869 | 11.026 | 1.00 | 50.00 | O |
| ATOM | 9718 | N | ASP | F | 486 | 14.326 | −40.981 | 13.243 | 1.00 | 50.00 | N |
| ATOM | 9719 | CA | ASP | F | 486 | 15.286 | −42.081 | 13.340 | 1.00 | 50.00 | C |
| ATOM | 9720 | C | ASP | F | 486 | 14.761 | −43.358 | 13.989 | 1.00 | 50.00 | C |
| ATOM | 9721 | O | ASP | F | 486 | 15.041 | −44.424 | 13.443 | 1.00 | 50.00 | O |
| ATOM | 9722 | CB | ASP | F | 486 | 16.578 | −41.614 | 14.012 | 1.00 | 50.00 | C |
| ATOM | 9723 | CG | ASP | F | 486 | 17.344 | −40.510 | 13.295 | 1.00 | 50.00 | C |
| ATOM | 9724 | OD1 | ASP | F | 486 | 17.992 | −40.791 | 12.264 | 1.00 | 50.00 | O |
| ATOM | 9725 | OD2 | ASP | F | 486 | 17.289 | −39.347 | 13.749 | 1.00 | 50.00 | O |
| ATOM | 9726 | N | GLU | F | 487 | 13.983 | −43.304 | 15.061 | 1.00 | 50.00 | N |
| ATOM | 9727 | CA | GLU | F | 487 | 13.142 | −44.417 | 15.508 | 1.00 | 50.00 | C |
| ATOM | 9728 | C | GLU | F | 487 | 12.213 | −44.952 | 14.426 | 1.00 | 50.00 | C |
| ATOM | 9729 | O | GLU | F | 487 | 12.231 | −46.157 | 14.180 | 1.00 | 50.00 | O |
| ATOM | 9730 | CB | GLU | F | 487 | 12.336 | −44.111 | 16.772 | 1.00 | 50.00 | C |
| ATOM | 9731 | CG | GLU | F | 487 | 11.572 | −45.277 | 17.402 | 1.00 | 50.00 | C |
| ATOM | 9732 | CD | GLU | F | 487 | 10.118 | −45.465 | 16.989 | 1.00 | 50.00 | C |
| ATOM | 9733 | OE1 | GLU | F | 487 | 9.847 | −45.990 | 15.888 | 1.00 | 50.00 | O |
| ATOM | 9734 | OE2 | GLU | F | 487 | 9.218 | −45.109 | 17.780 | 1.00 | 50.00 | O |
| ATOM | 9735 | N | PHE | F | 488 | 11.416 | −44.097 | 13.806 | 1.00 | 50.00 | N |
| ATOM | 9736 | CA | PHE | F | 488 | 10.547 | −44.484 | 12.697 | 1.00 | 50.00 | C |
| ATOM | 9737 | C | PHE | F | 488 | 11.202 | −45.270 | 11.567 | 1.00 | 50.00 | C |
| ATOM | 9738 | O | PHE | F | 488 | 10.699 | −46.337 | 11.220 | 1.00 | 50.00 | O |
| ATOM | 9739 | CB | PHE | F | 488 | 9.840 | −43.213 | 12.228 | 1.00 | 50.00 | C |
| ATOM | 9740 | CG | PHE | F | 488 | 8.834 | −43.589 | 11.165 | 1.00 | 50.00 | C |
| ATOM | 9741 | CD1 | PHE | F | 488 | 7.666 | −44.160 | 11.524 | 1.00 | 50.00 | C |
| ATOM | 9742 | CD2 | PHE | F | 488 | 9.144 | −43.451 | 9.860 | 1.00 | 50.00 | C |
| ATOM | 9743 | CE1 | PHE | F | 488 | 6.708 | −44.372 | 10.602 | 1.00 | 50.00 | C |
| ATOM | 9744 | CE2 | PHE | F | 488 | 8.214 | −43.724 | 8.924 | 1.00 | 50.00 | C |
| ATOM | 9745 | CZ | PHE | F | 488 | 6.983 | −44.118 | 9.308 | 1.00 | 50.00 | C |
| ATOM | 9746 | N | ASP | F | 489 | 12.260 | −44.739 | 10.976 | 1.00 | 50.00 | N |
| ATOM | 9747 | CA | ASP | F | 489 | 13.037 | −45.408 | 9.932 | 1.00 | 50.00 | C |
| ATOM | 9748 | C | ASP | F | 489 | 13.603 | −46.768 | 10.331 | 1.00 | 50.00 | C |
| ATOM | 9749 | O | ASP | F | 489 | 13.483 | −47.708 | 9.546 | 1.00 | 50.00 | O |
| ATOM | 9750 | CB | ASP | F | 489 | 14.111 | −44.448 | 9.419 | 1.00 | 50.00 | C |
| ATOM | 9751 | CG | ASP | F | 489 | 13.686 | −43.153 | 8.741 | 1.00 | 50.00 | C |
| ATOM | 9752 | OD1 | ASP | F | 489 | 12.515 | −43.006 | 8.327 | 1.00 | 50.00 | O |
| ATOM | 9753 | OD2 | ASP | F | 489 | 14.541 | −42.249 | 8.618 | 1.00 | 50.00 | O |
| ATOM | 9754 | N | ALA | F | 490 | 14.132 | −46.914 | 11.536 | 1.00 | 50.00 | N |
| ATOM | 9755 | CA | ALA | F | 490 | 14.432 | −48.200 | 12.167 | 1.00 | 50.00 | C |
| ATOM | 9756 | C | ALA | F | 490 | 13.285 | −49.182 | 12.383 | 1.00 | 50.00 | C |
| ATOM | 9757 | O | ALA | F | 490 | 13.466 | −50.355 | 12.058 | 1.00 | 50.00 | O |
| ATOM | 9758 | CB | ALA | F | 490 | 15.137 | −47.921 | 13.493 | 1.00 | 50.00 | C |
| ATOM | 9759 | N | SER | F | 491 | 12.138 | −48.766 | 12.897 | 1.00 | 50.00 | N |
| ATOM | 9760 | CA | SER | F | 491 | 10.922 | −49.577 | 12.979 | 1.00 | 50.00 | C |
| ATOM | 9761 | C | SER | F | 491 | 10.394 | −50.112 | 11.653 | 1.00 | 50.00 | C |
| ATOM | 9762 | O | SER | F | 491 | 10.050 | −51.289 | 11.567 | 1.00 | 50.00 | O |
| ATOM | 9763 | CB | SER | F | 491 | 9.794 | −48.822 | 13.681 | 1.00 | 50.00 | C |
| ATOM | 9764 | OG | SER | F | 491 | 10.082 | −48.641 | 15.060 | 1.00 | 50.00 | O |
| ATOM | 9765 | N | ILE | F | 492 | 10.367 | −49.294 | 10.615 | 1.00 | 50.00 | N |
| ATOM | 9766 | CA | ILE | F | 492 | 10.150 | −49.711 | 9.230 | 1.00 | 50.00 | C |
| ATOM | 9767 | C | ILE | F | 492 | 11.126 | −50.760 | 8.709 | 1.00 | 50.00 | C |
| ATOM | 9768 | O | ILE | F | 492 | 10.674 | −51.725 | 8.095 | 1.00 | 50.00 | O |
| ATOM | 9769 | CB | ILE | F | 492 | 10.094 | −48.434 | 8.390 | 1.00 | 50.00 | C |
| ATOM | 9770 | CG1 | ILE | F | 492 | 8.798 | −47.686 | 8.706 | 1.00 | 50.00 | C |
| ATOM | 9771 | CG2 | ILE | F | 492 | 10.362 | −48.701 | 6.909 | 1.00 | 50.00 | C |
| ATOM | 9772 | CD1 | ILE | F | 492 | 8.336 | −46.629 | 7.711 | 1.00 | 50.00 | C |
| ATOM | 9773 | N | SER | F | 493 | 12.423 | −50.536 | 8.831 | 1.00 | 50.00 | N |
| ATOM | 9774 | CA | SER | F | 493 | 13.437 | −51.533 | 8.488 | 1.00 | 50.00 | C |
| ATOM | 9775 | C | SER | F | 493 | 13.301 | −52.869 | 9.212 | 1.00 | 50.00 | C |
| ATOM | 9776 | O | SER | F | 493 | 13.481 | −53.898 | 8.564 | 1.00 | 50.00 | O |
| ATOM | 9777 | CB | SER | F | 493 | 14.811 | −50.886 | 8.654 | 1.00 | 50.00 | C |
| ATOM | 9778 | OG | SER | F | 493 | 15.816 | −51.728 | 8.108 | 1.00 | 50.00 | O |
| ATOM | 9779 | N | GLN | F | 494 | 12.902 | −52.882 | 10.475 | 1.00 | 50.00 | N |
| ATOM | 9780 | CA | GLN | F | 494 | 12.337 | −54.044 | 11.161 | 1.00 | 50.00 | C |
| ATOM | 9781 | C | GLN | F | 494 | 11.124 | −54.700 | 10.508 | 1.00 | 50.00 | C |
| ATOM | 9782 | O | GLN | F | 494 | 11.104 | −55.928 | 10.439 | 1.00 | 50.00 | O |
| ATOM | 9783 | CB | GLN | F | 494 | 12.058 | −53.661 | 12.615 | 1.00 | 50.00 | C |
| ATOM | 9784 | CG | GLN | F | 494 | 11.701 | −54.818 | 13.547 | 1.00 | 50.00 | C |
| ATOM | 9785 | CD | GLN | F | 494 | 11.317 | −54.429 | 14.967 | 1.00 | 50.00 | C |
| ATOM | 9786 | OE1 | GLN | F | 494 | 11.247 | −53.264 | 15.358 | 1.00 | 50.00 | O |
| ATOM | 9787 | NE2 | GLN | F | 494 | 11.032 | −55.428 | 15.782 | 1.00 | 50.00 | N |
| ATOM | 9788 | N | VAL | F | 495 | 10.132 | −53.961 | 10.038 | 1.00 | 50.00 | N |
| ATOM | 9789 | CA | VAL | F | 495 | 9.022 | −54.487 | 9.241 | 1.00 | 50.00 | C |
| ATOM | 9790 | C | VAL | F | 495 | 9.485 | −55.115 | 7.929 | 1.00 | 50.00 | C |
| ATOM | 9791 | O | VAL | F | 495 | 9.207 | −56.298 | 7.741 | 1.00 | 50.00 | O |
| ATOM | 9792 | CB | VAL | F | 495 | 7.911 | −53.453 | 9.044 | 1.00 | 50.00 | C |
| ATOM | 9793 | CG1 | VAL | F | 495 | 6.719 | −54.047 | 8.295 | 1.00 | 50.00 | C |
| ATOM | 9794 | CG2 | VAL | F | 495 | 7.318 | −52.723 | 10.249 | 1.00 | 50.00 | C |
| ATOM | 9795 | N | ASN | F | 496 | 10.179 | −54.416 | 7.043 | 1.00 | 50.00 | N |
| ATOM | 9796 | CA | ASN | F | 496 | 10.823 | −54.980 | 5.855 | 1.00 | 50.00 | C |
| ATOM | 9797 | C | ASN | F | 496 | 11.701 | −56.210 | 6.069 | 1.00 | 50.00 | C |
| ATOM | 9798 | O | ASN | F | 496 | 11.618 | −57.140 | 5.267 | 1.00 | 50.00 | O |
| ATOM | 9799 | CB | ASN | F | 496 | 11.575 | −53.895 | 5.082 | 1.00 | 50.00 | C |
| ATOM | 9800 | CG | ASN | F | 496 | 10.733 | −52.807 | 4.428 | 1.00 | 50.00 | C |
| ATOM | 9801 | OD1 | ASN | F | 496 | 9.849 | −53.079 | 3.617 | 1.00 | 50.00 | O |
| ATOM | 9802 | ND2 | ASN | F | 496 | 10.986 | −51.547 | 4.733 | 1.00 | 50.00 | N |
| ATOM | 9803 | N | GLU | F | 497 | 12.462 | −56.277 | 7.150 | 1.00 | 50.00 | N |
| ATOM | 9804 | CA | GLU | F | 497 | 13.084 | −57.496 | 7.668 | 1.00 | 50.00 | C |
| ATOM | 9805 | C | GLU | F | 497 | 12.124 | −58.639 | 7.982 | 1.00 | 50.00 | C |
| ATOM | 9806 | O | GLU | F | 497 | 12.334 | −59.716 | 7.427 | 1.00 | 50.00 | O |
| ATOM | 9807 | CB | GLU | F | 497 | 13.967 | −57.137 | 8.864 | 1.00 | 50.00 | C |
| ATOM | 9808 | CG | GLU | F | 497 | 14.930 | −58.236 | 9.310 | 1.00 | 50.00 | C |
| ATOM | 9809 | CD | GLU | F | 497 | 15.751 | −57.868 | 10.537 | 1.00 | 50.00 | C |
| ATOM | 9810 | OE1 | GLU | F | 497 | 16.532 | −56.894 | 10.477 | 1.00 | 50.00 | O |
| ATOM | 9811 | OE2 | GLU | F | 497 | 15.628 | −58.557 | 11.573 | 1.00 | 50.00 | O |
| ATOM | 9812 | N | LYS | F | 498 | 11.096 | −58.458 | 8.796 | 1.00 | 50.00 | N |
| ATOM | 9813 | CA | LYS | F | 498 | 10.030 | −59.441 | 9.011 | 1.00 | 50.00 | C |
| ATOM | 9814 | C | LYS | F | 498 | 9.299 | −60.023 | 7.807 | 1.00 | 50.00 | C |
| ATOM | 9815 | O | LYS | F | 498 | 8.840 | −61.165 | 7.842 | 1.00 | 50.00 | O |
| ATOM | 9816 | CB | LYS | F | 498 | 9.004 | −58.915 | 10.017 | 1.00 | 50.00 | C |
| ATOM | 9817 | CG | LYS | F | 498 | 9.466 | −58.843 | 11.472 | 1.00 | 50.00 | C |
| ATOM | 9818 | CD | LYS | F | 498 | 9.738 | −60.216 | 12.085 | 1.00 | 50.00 | C |
| ATOM | 9819 | CE | LYS | F | 498 | 10.476 | −60.154 | 13.419 | 1.00 | 50.00 | C |
| ATOM | 9820 | NZ | LYS | F | 498 | 10.840 | −61.514 | 13.844 | 1.00 | 50.00 | N |
| ATOM | 9821 | N | ILE | F | 499 | 9.215 | −59.250 | 6.739 | 1.00 | 50.00 | N |
| ATOM | 9822 | CA | ILE | F | 499 | 8.639 | −59.641 | 5.453 | 1.00 | 50.00 | C |
| ATOM | 9823 | C | ILE | F | 499 | 9.617 | −60.495 | 4.656 | 1.00 | 50.00 | C |
| ATOM | 9824 | O | ILE | F | 499 | 9.203 | −61.551 | 4.181 | 1.00 | 50.00 | O |
| ATOM | 9825 | CB | ILE | F | 499 | 8.172 | −58.379 | 4.726 | 1.00 | 50.00 | C |
| ATOM | 9826 | CG1 | ILE | F | 499 | 7.133 | −57.667 | 5.596 | 1.00 | 50.00 | C |
| ATOM | 9827 | CG2 | ILE | F | 499 | 7.718 | −58.677 | 3.297 | 1.00 | 50.00 | C |
| ATOM | 9828 | CD1 | ILE | F | 499 | 6.850 | −56.241 | 5.148 | 1.00 | 50.00 | C |
| ATOM | 9829 | N | ASN | F | 500 | 10.856 | −60.070 | 4.470 | 1.00 | 50.00 | N |
| ATOM | 9830 | CA | ASN | F | 500 | 11.924 | −60.910 | 3.926 | 1.00 | 50.00 | C |
| ATOM | 9831 | C | ASN | F | 500 | 12.171 | −62.220 | 4.668 | 1.00 | 50.00 | C |
| ATOM | 9832 | O | ASN | F | 500 | 12.320 | −63.238 | 3.993 | 1.00 | 50.00 | O |
| ATOM | 9833 | CB | ASN | F | 500 | 13.193 | −60.071 | 3.767 | 1.00 | 50.00 | C |
| ATOM | 9834 | CG | ASN | F | 500 | 13.160 | −58.972 | 2.713 | 1.00 | 50.00 | C |
| ATOM | 9835 | OD1 | ASN | F | 500 | 12.676 | −59.157 | 1.597 | 1.00 | 50.00 | O |
| ATOM | 9836 | ND2 | ASN | F | 500 | 13.686 | −57.800 | 3.017 | 1.00 | 50.00 | N |
| ATOM | 9837 | N | GLN | F | 501 | 12.129 | −62.255 | 5.991 | 1.00 | 50.00 | N |
| ATOM | 9838 | CA | GLN | F | 501 | 11.968 | −63.469 | 6.795 | 1.00 | 50.00 | C |
| ATOM | 9839 | C | GLN | F | 501 | 10.778 | −64.354 | 6.444 | 1.00 | 50.00 | C |
| ATOM | 9840 | O | GLN | F | 501 | 10.970 | −65.557 | 6.271 | 1.00 | 50.00 | O |
| ATOM | 9841 | CB | GLN | F | 501 | 11.850 | −63.175 | 8.291 | 1.00 | 50.00 | C |
| ATOM | 9842 | CG | GLN | F | 501 | 13.070 | −62.604 | 9.011 | 1.00 | 50.00 | C |
| ATOM | 9843 | CD | GLN | F | 501 | 12.778 | −62.344 | 10.483 | 1.00 | 50.00 | C |
| ATOM | 9844 | OE1 | GLN | F | 501 | 12.247 | −63.190 | 11.201 | 1.00 | 50.00 | O |
| ATOM | 9845 | NE2 | GLN | F | 501 | 13.102 | −61.165 | 10.981 | 1.00 | 50.00 | N |
| ATOM | 9846 | N | SER | F | 502 | 9.578 | −63.805 | 6.340 | 1.00 | 50.00 | N |
| ATOM | 9847 | CA | SER | F | 502 | 8.413 | −64.514 | 5.815 | 1.00 | 50.00 | C |
| ATOM | 9848 | C | SER | F | 502 | 8.602 | −65.101 | 4.421 | 1.00 | 50.00 | C |
| ATOM | 9849 | O | SER | F | 502 | 8.504 | −66.321 | 4.305 | 1.00 | 50.00 | O |
| ATOM | 9850 | CB | SER | F | 502 | 7.193 | −63.601 | 5.893 | 1.00 | 50.00 | C |
| ATOM | 9851 | OG | SER | F | 502 | 6.087 | −64.373 | 5.451 | 1.00 | 50.00 | O |
| ATOM | 9852 | N | LEU | F | 503 | 8.942 | −64.314 | 3.412 | 1.00 | 50.00 | N |
| ATOM | 9853 | CA | LEU | F | 503 | 9.369 | −64.789 | 2.095 | 1.00 | 50.00 | C |
| ATOM | 9854 | C | LEU | F | 503 | 10.411 | −65.904 | 2.061 | 1.00 | 50.00 | C |
| ATOM | 9855 | O | LEU | F | 503 | 10.266 | −66.817 | 1.249 | 1.00 | 50.00 | O |
| ATOM | 9856 | CB | LEU | F | 503 | 9.778 | −63.607 | 1.213 | 1.00 | 50.00 | C |
| ATOM | 9857 | CG | LEU | F | 503 | 8.767 | −62.490 | 0.943 | 1.00 | 50.00 | C |
| ATOM | 9858 | CD1 | LEU | F | 503 | 9.343 | −61.375 | 0.072 | 1.00 | 50.00 | C |
| ATOM | 9859 | CD2 | LEU | F | 503 | 7.495 | −63.018 | 0.285 | 1.00 | 50.00 | C |
| ATOM | 9860 | N | ALA | F | 504 | 11.392 | −65.889 | 2.949 | 1.00 | 50.00 | N |
| ATOM | 9861 | CA | ALA | F | 504 | 12.309 | −66.999 | 3.210 | 1.00 | 50.00 | C |
| ATOM | 9862 | C | ALA | F | 504 | 11.725 | −68.263 | 3.831 | 1.00 | 50.00 | C |
| ATOM | 9863 | O | ALA | F | 504 | 11.995 | −69.332 | 3.284 | 1.00 | 50.00 | O |
| ATOM | 9864 | CB | ALA | F | 504 | 13.463 | −66.464 | 4.056 | 1.00 | 50.00 | C |
| ATOM | 9865 | N | PHE | F | 505 | 10.938 | −68.211 | 4.894 | 1.00 | 50.00 | N |
| ATOM | 9866 | CA | PHE | F | 505 | 10.156 | −69.349 | 5.386 | 1.00 | 50.00 | C |
| ATOM | 9867 | C | PHE | F | 505 | 9.251 | −70.070 | 4.394 | 1.00 | 50.00 | C |
| ATOM | 9868 | O | PHE | F | 505 | 9.096 | −71.289 | 4.456 | 1.00 | 50.00 | O |
| ATOM | 9869 | CB | PHE | F | 505 | 9.353 | −68.984 | 6.638 | 1.00 | 50.00 | C |
| ATOM | 9870 | CG | PHE | F | 505 | 10.119 | −68.853 | 7.937 | 1.00 | 50.00 | C |
| ATOM | 9871 | CD1 | PHE | F | 505 | 9.996 | −67.751 | 8.705 | 1.00 | 50.00 | C |
| ATOM | 9872 | CD2 | PHE | F | 505 | 10.930 | −69.853 | 8.342 | 1.00 | 50.00 | C |
| ATOM | 9873 | CE1 | PHE | F | 505 | 10.711 | −67.629 | 9.842 | 1.00 | 50.00 | C |
| ATOM | 9874 | CE2 | PHE | F | 505 | 11.662 | −69.729 | 9.467 | 1.00 | 50.00 | C |
| ATOM | 9875 | CZ | PHE | F | 505 | 11.552 | −68.614 | 10.217 | 1.00 | 50.00 | C |
| ATOM | 9876 | N | ILE | F | 506 | 8.687 | −69.320 | 3.464 | 1.00 | 50.00 | N |
| ATOM | 9877 | CA | ILE | F | 506 | 7.882 | −69.821 | 2.351 | 1.00 | 50.00 | C |
| ATOM | 9878 | C | ILE | F | 506 | 8.737 | −70.423 | 1.244 | 1.00 | 50.00 | C |
| ATOM | 9879 | O | ILE | F | 506 | 8.401 | −71.519 | 0.797 | 1.00 | 50.00 | O |
| ATOM | 9880 | CB | ILE | F | 506 | 6.892 | −68.750 | 1.887 | 1.00 | 50.00 | C |
| ATOM | 9881 | CG1 | ILE | F | 506 | 6.095 | −68.162 | 3.058 | 1.00 | 50.00 | C |
| ATOM | 9882 | CG2 | ILE | F | 506 | 6.165 | −69.279 | 0.647 | 1.00 | 50.00 | C |
| ATOM | 9883 | CD1 | ILE | F | 506 | 5.413 | −69.198 | 3.942 | 1.00 | 50.00 | C |
| ATOM | 9884 | N | ARG | F | 507 | 9.773 | −69.744 | 0.780 | 1.00 | 50.00 | N |
| ATOM | 9885 | CA | ARG | F | 507 | 10.796 | −70.339 | −0.080 | 1.00 | 50.00 | C |
| ATOM | 9886 | C | ARG | F | 507 | 11.291 | −71.718 | 0.345 | 1.00 | 50.00 | C |
| ATOM | 9887 | O | ARG | F | 507 | 11.325 | −72.614 | −0.497 | 1.00 | 50.00 | O |
| ATOM | 9888 | CB | ARG | F | 507 | 11.921 | −69.316 | −0.238 | 1.00 | 50.00 | C |
| ATOM | 9889 | CG | ARG | F | 507 | 12.818 | −69.642 | −1.430 | 1.00 | 50.00 | C |
| ATOM | 9890 | CD | ARG | F | 507 | 14.124 | −68.852 | −1.494 | 1.00 | 50.00 | C |
| ATOM | 9891 | NE | ARG | F | 507 | 14.788 | −68.958 | −2.794 | 1.00 | 50.00 | N |
| ATOM | 9892 | CZ | ARG | F | 507 | 15.247 | −70.070 | −3.345 | 1.00 | 50.00 | C |
| ATOM | 9893 | NH1 | ARG | F | 507 | 15.261 | −71.252 | −2.755 | 1.00 | 50.00 | N |
| ATOM | 9894 | NH2 | ARG | F | 507 | 15.708 | −70.001 | −4.580 | 1.00 | 50.00 | N |
| ATOM | 9895 | N | LYS | F | 508 | 11.576 | −71.921 | 1.621 | 1.00 | 50.00 | N |
| ATOM | 9896 | CA | LYS | F | 508 | 11.861 | −73.231 | 2.208 | 1.00 | 50.00 | C |
| ATOM | 9897 | C | LYS | F | 508 | 10.745 | −74.267 | 2.175 | 1.00 | 50.00 | C |
| ATOM | 9898 | O | LYS | F | 508 | 11.039 | −75.433 | 1.917 | 1.00 | 50.00 | O |
| ATOM | 9899 | CB | LYS | F | 508 | 12.328 | −73.083 | 3.655 | 1.00 | 50.00 | C |
| ATOM | 9900 | CG | LYS | F | 508 | 13.669 | −72.364 | 3.778 | 1.00 | 50.00 | C |
| ATOM | 9901 | CD | LYS | F | 508 | 14.274 | −72.506 | 5.172 | 1.00 | 50.00 | C |
| ATOM | 9902 | CE | LYS | F | 508 | 15.659 | −71.864 | 5.210 | 1.00 | 50.00 | C |
| ATOM | 9903 | NZ | LYS | F | 508 | 16.279 | −72.081 | 6.525 | 1.00 | 50.00 | N |
| ATOM | 9904 | N | SER | F | 509 | 9.501 | −73.905 | 2.436 | 1.00 | 50.00 | N |
| ATOM | 9905 | CA | SER | F | 509 | 8.386 | −74.832 | 2.247 | 1.00 | 50.00 | C |
| ATOM | 9906 | C | SER | F | 509 | 8.026 | −75.180 | 0.808 | 1.00 | 50.00 | C |
| ATOM | 9907 | O | SER | F | 509 | 7.850 | −76.370 | 0.551 | 1.00 | 50.00 | O |
| ATOM | 9908 | CB | SER | F | 509 | 7.152 | −74.307 | 2.970 | 1.00 | 50.00 | C |
| ATOM | 9909 | OG | SER | F | 509 | 6.291 | −75.425 | 3.132 | 1.00 | 50.00 | O |
| ATOM | 9910 | N | ASP | F | 510 | 7.955 | −74.237 | −0.119 | 1.00 | 50.00 | N |
| ATOM | 9911 | CA | ASP | F | 510 | 7.959 | −74.506 | −1.559 | 1.00 | 50.00 | C |
| ATOM | 9912 | C | ASP | F | 510 | 9.042 | −75.483 | −2.009 | 1.00 | 50.00 | C |
| ATOM | 9913 | O | ASP | F | 510 | 8.691 | −76.489 | −2.623 | 1.00 | 50.00 | O |
| ATOM | 9914 | CB | ASP | F | 510 | 7.978 | −73.229 | −2.401 | 1.00 | 50.00 | C |
| ATOM | 9915 | CG | ASP | F | 510 | 6.851 | −72.209 | −2.321 | 1.00 | 50.00 | C |
| ATOM | 9916 | OD1 | ASP | F | 510 | 5.768 | −72.425 | −2.908 | 1.00 | 50.00 | O |
| ATOM | 9917 | OD2 | ASP | F | 510 | 7.065 | −71.126 | −1.735 | 1.00 | 50.00 | O |
| ATOM | 9918 | N | GLU | F | 511 | 10.295 | −75.290 | −1.623 | 1.00 | 50.00 | N |
| ATOM | 9919 | CA | GLU | F | 511 | 11.379 | −76.268 | −1.735 | 1.00 | 50.00 | C |
| ATOM | 9920 | C | GLU | F | 511 | 11.089 | −77.686 | −1.254 | 1.00 | 50.00 | C |
| ATOM | 9921 | O | GLU | F | 511 | 11.154 | −78.594 | −2.082 | 1.00 | 50.00 | O |
| ATOM | 9922 | CB | GLU | F | 511 | 12.640 | −75.760 | −1.033 | 1.00 | 50.00 | C |
| ATOM | 9923 | CG | GLU | F | 511 | 13.452 | −74.643 | −1.685 | 1.00 | 50.00 | C |
| ATOM | 9924 | CD | GLU | F | 511 | 14.527 | −74.134 | −0.736 | 1.00 | 50.00 | C |
| ATOM | 9925 | OE1 | GLU | F | 511 | 15.449 | −74.906 | −0.393 | 1.00 | 50.00 | O |
| ATOM | 9926 | OE2 | GLU | F | 511 | 14.449 | −72.965 | −0.299 | 1.00 | 50.00 | O |
| ATOM | 9927 | N | LEU | F | 512 | 10.764 | −77.898 | 0.012 | 1.00 | 50.00 | N |
| ATOM | 9928 | CA | LEU | F | 512 | 10.348 | −79.188 | 0.567 | 1.00 | 50.00 | C |
| ATOM | 9929 | C | LEU | F | 512 | 9.299 | −79.987 | −0.198 | 1.00 | 50.00 | C |
| ATOM | 9930 | O | LEU | F | 512 | 9.360 | −81.213 | −0.279 | 1.00 | 50.00 | O |
| ATOM | 9931 | CB | LEU | F | 512 | 9.867 | −79.025 | 2.011 | 1.00 | 50.00 | C |
| ATOM | 9932 | CG | LEU | F | 512 | 10.907 | −78.666 | 3.072 | 1.00 | 50.00 | C |
| ATOM | 9933 | CD1 | LEU | F | 512 | 10.242 | −78.387 | 4.419 | 1.00 | 50.00 | C |
| ATOM | 9934 | CD2 | LEU | F | 512 | 11.984 | −79.739 | 3.224 | 1.00 | 50.00 | C |
| ATOM | 9935 | N | LEU | F | 513 | 8.333 | −79.280 | −0.756 | 1.00 | 50.00 | N |
| ATOM | 9936 | CA | LEU | F | 513 | 7.212 | −79.856 | −1.496 | 1.00 | 50.00 | C |
| ATOM | 9937 | C | LEU | F | 513 | 7.479 | −80.238 | −2.946 | 1.00 | 50.00 | C |
| ATOM | 9938 | O | LEU | F | 513 | 7.071 | −81.309 | −3.394 | 1.00 | 50.00 | O |
| ATOM | 9939 | CB | LEU | F | 513 | 6.079 | −78.838 | −1.410 | 1.00 | 50.00 | C |
| ATOM | 9940 | CG | LEU | F | 513 | 5.645 | −78.525 | 0.023 | 1.00 | 50.00 | C |
| ATOM | 9941 | CD1 | LEU | F | 513 | 4.845 | −77.239 | −0.105 | 1.00 | 50.00 | C |
| ATOM | 9942 | CD2 | LEU | F | 513 | 4.709 | −79.553 | 0.644 | 1.00 | 50.00 | C |
| ATOM | 9943 | N | HIS | F | 514 | 8.214 | −79.418 | −3.677 | 1.00 | 50.00 | N |
| ATOM | 9944 | CA | HIS | F | 514 | 8.874 | −79.837 | −4.913 | 1.00 | 50.00 | C |
| ATOM | 9945 | C | HIS | F | 514 | 9.867 | −80.992 | −4.801 | 1.00 | 50.00 | C |
| ATOM | 9946 | O | HIS | F | 514 | 10.126 | −81.619 | −5.828 | 1.00 | 50.00 | O |
| ATOM | 9947 | CB | HIS | F | 514 | 9.435 | −78.591 | −5.601 | 1.00 | 50.00 | C |
| ATOM | 9948 | CG | HIS | F | 514 | 8.456 | −77.504 | −5.993 | 1.00 | 50.00 | C |
| ATOM | 9949 | ND1 | HIS | F | 514 | 7.279 | −77.726 | −6.682 | 1.00 | 50.00 | N |
| ATOM | 9950 | CD2 | HIS | F | 514 | 8.559 | −76.169 | −5.609 | 1.00 | 50.00 | C |
| ATOM | 9951 | CE1 | HIS | F | 514 | 6.744 | −76.456 | −6.623 | 1.00 | 50.00 | C |
| ATOM | 9952 | NE2 | HIS | F | 514 | 7.446 | −75.450 | −5.995 | 1.00 | 50.00 | N |
| ATOM | 9953 | N | ASN | F | 515 | 10.360 | −81.361 | −3.628 | 1.00 | 50.00 | N |
| ATOM | 9954 | CA | ASN | F | 515 | 11.135 | −82.577 | −3.380 | 1.00 | 50.00 | C |
| ATOM | 9955 | C | ASN | F | 515 | 10.247 | −83.812 | −3.472 | 1.00 | 50.00 | C |
| ATOM | 9956 | O | ASN | F | 515 | 9.752 | −84.399 | −2.511 | 1.00 | 50.00 | O |
| ATOM | 9957 | CB | ASN | F | 515 | 11.863 | −82.447 | −2.041 | 1.00 | 50.00 | C |
| ATOM | 9958 | CG | ASN | F | 515 | 12.921 | −83.501 | −1.749 | 1.00 | 50.00 | C |
| ATOM | 9959 | OD1 | ASN | F | 515 | 12.612 | −84.631 | −1.373 | 1.00 | 50.00 | O |
| ATOM | 9960 | ND2 | ASN | F | 515 | 14.192 | −83.173 | −1.894 | 1.00 | 50.00 | N |
| END | | | | | | | | | | | |

TABLE 3
Summary of mutagenesis experiments used to characterise Site A.

| Mutation^{a} | Protein site^{b} | Activity^{c} | Comp. 1^{d} | Comp. 2^{d} |
|---|---|---|---|---|
| S319T | Site A | Inactive | N/A | N/A |
| S319L | Site A | Active | Resistant | Resistant |
| N371Q | Site A | Active | NT | Weak R |
| V4061 | Site A | Very Active | NT | Sensitive |
| G51A | Site A | Inactive | N/A | N/A |
| G51V | Site A | Inactive | N/A | N/A |
| F366W | Site A | Weakly Active | Weak R. | NT |
| D368E | Site A | V. Weakly Active | Sensitive | NT |
| S362T | Site A | Active | Sensitive | NT |
| E92D | Other | Inactive | N/A | N/A |
| V281F | Other | Inactive | N/A | N/A |
| A241L | Other | V. Weakly Active | Sensitive | NT |
| T50L | Other | Inactive | N/A | N/A |
| T326L | Other | Active | Resistant | Sensitive |
| G51V/E92D | Sites A, | V. Weakly Active | Sensitive | NT |
| | other | | | |
| S362T/S398L | Site A | Inactive | N/A | N/A |
| N317Q/S398L | Site A | Weakly Active | NT | Sensitive |

^{a}Site-specific mutation made in RSV-F sequence.
^{b}Position of the mutation either in Site A itself or elsewhere within the RSV-F structure.
^{c}Activity of the recombinant RSV-F mutant in the standard syncytia formation assay detailed herein.
^{d}Resistance or sensitivity of recombinant RSV-F protein carrying the specific mutation in the standard syncytia formation assay.
N/A: not active.
NT: not tested.

TABLE 4
Selection and Characterisation of Drug Resistant RSV A (Long strain).

| COMPOUND 2 | | Mutations observed | Mutations observed virus |
|---|---|---|---|
| concentration | | in original | grown up from selected |
| used for selection | Isolate | selected virus | virus for assay purposes |
| 0.1 μg/ml | 1 | L138I, S319F & H515N | E487D & H515N |
| | 2 | L138I & H515N | E487D & H515N |
| | 3 | K399N & T523I | K399N & T523I |
| 1 μg/ml | 4 | D489Y | D489Y |
| 10 μg/ml | 5 | L138I | L1381 |
| | 6 | D489Y | D489Y |
| | 7 | D486V | In progress |
| | 8 | R136S & L141F | In progress |
| | 9 | D489Y | In progress |

Cross resistance data:

| | EC50 (ng/ml) | | |
|---|---|---|---|
| | RSV Long | RSV Long mut | RSV Long mut |
| Drug | wild type | L138I | D489Y |
| COMPOUND 2 | <5 | >10,000 | >10,000 |
| COMPOUND 1 | <5 | >10,000 | >10,000 |
| Ribavirin* | 5536 | —1897 | |

*_(1-D-ribofuranosyl-1H-1,2,4-triazole-3-carboxamide)

### EXAMPLE 2

The following provides test examples of the application of the present invention.

### TEST EXAMPLE 1

Computational Prediction of the Binding Orientation of Fusion Inhibitors

Three known fusion inhibitors that act against RSV-F, ie COMPOUNDS 1-3 (FIG. 2) were computationally docked into the RSV-F model to clarify the interactions occurring between the drugs and the protein. Docking analysis of RSV-F was carried out using the program AutoDock 3.0.3 running on an SGI O2 workstation.

The algorithm employed by AutoDock is described in detail in the AutoDock User Guide distributed with the software and references therein (Morris, G. M. et al., supra), all of which are to be regarded as incorporated herein by reference. In brief, the region of space around the protein target selected for analysis is divided into a grid of points spaced a user-definable distance apart. At each of these points, the potential energy of every type of atom in the ligand being docked is calculated and stored in a potential map. During the actual docking process, the ligand is inserted into the assigned region of space and translated, rotated and allowed to alter user-selected torsion angles and the energy of the ligand rapidly calculated by reference to the stored maps of potentials, rather than direct calculation during the docking itself. After a number of steps of optimisation determined by the user, the best conformation are saved and the process repeated. After the desired number of conformations are generated they are grouped into “clusters” of similar structures, defined by a user selected RMS (Root Mean Square deviation in atomic position) tolerance.

Each of the three inhibitors was manually constructed in the Builder module of InsightII (Accelrys, Inc., Burlington, Mass., USA). After geometry optimisation within InsightII the ligands were saved as Accelrys CAR format files. These were converted to PDB format using the AutoDock utility program CARTOPDBQ. Rotatable bonds in the molecule were defined using the AutoDock utility program AUTOTORS to generate an AutoDock ligand input file given the extension PDBQT, containing atom charge information and definitions of rotatable bonds as well as the position and type of each atom. The minimised RSV-F model in Accelrys CAR format was converted to an AutoDock PDBQ file using the CARTOPDBQ utility. AutoDock solvation parameters were then added to the PDBQ format file using the AutoDock utility program ADDSOL, creating an AutoDock macromolecule input file given the extension PDBQS, containing atom charge and simple solvation model information as well as the position and type of each atom. Parameter files for generating the appropriate atom type grids for each ligand were then created using the AutoDock MKGPF3 program. This takes a ligand in PDBQT format and a macromolecule in PDBQS format and generates a grid parameter file (GPF) containing the required information to define the various energy grids needed to match the ligand with the target macromolecule. The GPF also contains positional information, defining the extent (in number of grid points), size (in angstrom per grid point) and precise position (as a set of XYZ coordinates in the same reference frame as the target macromolecule) of the chosen docking site. As the three ligands being analysed all contain the same set of atom types (C, N, O, and H) only a single GPF was required. The target site in RSV-F was selected as the center point in Site A, determined by averaging the coordinates of residues contributing to the site. A grid size of 0.375 Å, with 60 grid points in each dimension was used to fully define the grid position. The grid files were then generated using the AutoDock utility program AUTOGRID3, which takes as input a GPF file and the associated macromolecular PDBQS file and produces a set of grid maps (MAP files) defining the interaction energy at every point within the grid, one for each atom sort and one for electrostatic interactions.

A docking parameter file (DPF) was created for each ligand using the program MKDPF3. This takes a ligand in PDBQT format and a macromolecule in PDBQS format and generates a DPF which contains the required information for carrying out the docking analysis, including the names of various input and output files and the desired details of the docking process. The docking process was set to use the Genetic Algorithm option, with a total of 100 docked structures determined. Docking was carried out using the AUTODOCK3 program, which takes the DPF file and the associated PDBQT, PDBQS and MAP files and carries out the actual docking process, generating a docking log (DLG file) containing complete details of the docking process, the energies and final coordinates of the docked ligand structures.

The 100 docked structures for each ligand were clustered by AutoDock, as part of the docking process, into groups of structures with an RMSD less than a user-defined tolerance value. The clusters were then ranked on the energy of the best dock within each duster. FIG. 7 shows the best docking duster for each of the three ligands, clearly defining the orientation of each compound in Site A and the nature of the contacts that exist between the protein and the ligands.

### TEST EXAMPLE 2

Structure-Activity Relationship (SAR) Analysis by Characterization of Drug Binding Orientation

Detailed analyses of the contacts arising between RSV-F inhibitors and the protein can be determined by careful examination of the docked conformation of the inhibitor to the model. Correlation of the contacts between the protein atoms and regions of the drug with traditional SAR arising from activity in an in vitro assay can be made, allowing rationalisation of the observed SAR properties of the compounds and prediction of other SAR information. For this procedure, a model of the ligand—protein complex must be prepared in some manner such as that described above in Test Example 1. Interactions between the protein and ligand can then be measured in a variety of ways including, but not limited to, the following.

Firstly, the distances between each atom of the ligand and those atoms of the protein within a distance corresponding to effective physical contact, for example 3 Å, can be measured with a simple computer program written to calculate the distances between all atoms in two bound molecules and produce a list of adjacent atoms as output Secondly, the nature of the interactions between the ligand and protein in the complex can be defined by a graphical representation such as that generated by the program LIGPLOT (Wallace, A C et al., Protein Eng. 2:127-34, 1995). Thirdly, the interactions between the molecules can be examined visually using a computer with appropriate visual display hardware, preferably including the ability to display three-dimensional images using an appropriate stereo-viewing adaptor, and molecular graphics software such as Sybyl (Tripos Associates, St. Louis, Mo., USA), InsightII (Accelrys, Inc., Burlington, Mass., USA), VMD (Humphrey, W et al., J. Molec. Graphics. 14:33-38,1996), Vida (OpenEye, Santa Fe, N. Mex., USA) or the like which can calculate and display important contacts such as hydrogen bonds between the protein and ligand.

On the basis of one or more of these methods for analysing contacts between the ligand and protein details of the types of interactions that stabilise the complex, for example hydrogen bonds, favourable ionic interactions and the burial of exposed hydrophobic surfaces, or that destabilise the complex, for example repulsive ionic interactions, steric hindrance and restriction of mobile groups can be determined. Modifications of the chemistry of the ligand can then be proposed to increase the stabilising interactions and/or decrease the unfavourable interactions. These modified molecules can also be subjected to the same process of docking and examination independently, to ascertain whether the proposed modifications are likely to have the desired effect or instead cause the ligand to bind to the protein in a different, and potentially undesirable, manner.

### TEST EXAMPLE 3

De novo Design of Novel Fusion Inhibitors by Property-Based Analysis of Drug Binding Sites

A structure for the site of action of known inhibitors of RSV-F activity allows the de novo design of novel compounds with complementary properties to the site. This allows new fusion inhibitors to be devised without recourse to extensive in vitro screening of libraries of compounds. This process can be carried out with, for example, the program LUDI (Accelrys, Inc., Burlington, Mass., USA) or LeapFrog (Tripos Associates, St. Louis, Mo., USA) or other similar programs written for the purpose of de novo drug design.

LUDI is described in detail in the original paper (Bohm, H.-J. supra) and the supplied manual (Accelrys), all of which is to be regarded as incorporated herein by reference. In brief, LUDI functions by fitting small molecular fragments from an in silico library to optimal positions within a defined site on the surface of a target protein. These separate fragments are then joined by appropriate chemical linkers, again selected from an in silico library of potential linking chemistries. Alternately, new fragments can be added to existing fragments in such a way as to allow the two fragments to fuse into a single larger entity without the need for an additional linker.

Site A of RSV-F can be used as a target for LUDI to design novel fusion inhibitors. For instance, a LUDI run can be initiated by selecting a point within the site of interest, for example the gamma-2 carbon of residue Valine 406 in the RSV-F model, as the centre point for a sphere of user-defied radius within which LUDI identifies appropriate positions for fragments from the library. A series of parameters can then be set by the user including the scoring function used to rank the fragment positions, the number of bonds allowed to rotate during conformational searches of flexible ligands, the minimum and maximum values for various interactions such as hydrogen bonds and contact surface areas and the like. A set of initial fragment hits can then be generated, ranked by the scoring function selected by the user. After this, the structure of the protein containing each of these initial hits can be used as the basis for additional rounds of LUDI screening, building a larger ligand at each step. When a molecule of appropriate size for a lead candidate has been achieved (eg 500 to 1000 Daltons), the molecule can be saved and provided to appropriately skilled chemists as a synthetic target.

### TEST EXAMPLE 4

Screening in Silico Compound Libraries for Novel Fusion Inhibitors

Selection of compounds from an in silico library with complementarity to a site on a target protein can be carried out using a variety of software such as Cerius2 (available from Accelrys, Inc., Burlington, Mass., USA), FlexX (available from Tripos Associates, St. Louis, Mo., USA), FRED (available from OpenEye, Santa Fe, N. Mex., USA), or other similar programs written for the purpose of in silico screening. Libraries of small molecules can be obtained from a number of small molecule supply companies such as Maybridge (Tintagel, Cornwall, UK), Chembridge (San Diego, Calif., USA), InterBioscreen (Chernogolovka, Russia) or SPECS (Rijswijk, The Netherlands). These libraries provide a source of potential ligands which can be screened against the target site.

Site A of RSV-F can be employed for the purposes of in silico screening to identify potential inhibitors of RSV-F activity. For example, in Cerius2, the screening process involves creating a series of “queries” in a sphere centred on a position selected by the user. These queries are generated using the Query Builder subprogram which examines the volume of space contained within the sphere to locate and cluster particular properties, such as hydrogen bond donors and acceptors and lipophilic areas. A set of volume exclusions are also prepared to prevent selected ligands overlapping with atoms from the protein. The property clusters produced by Query Builder are used for screening the library, such that compounds selected from the library must satisfy a user-defined number of the interactions apparent in the property clusters. The compounds selected in this manner can then be scored using a number of scoring functions to rank their fit to the protein site. Compounds selected in this way can then be obtained from their respective suppliers and tested for anti-fusion activity in in vitro assays of RSV-F activity.

Identification of antigenic regions likely to give rise to neutralizing antibodies Another use for the RSV-F structure model of the present invention is the rational selection of regions within the RSV-F protein that are likely to give rise to RSV-binding and/or RSV-neutralising antibodies if employed as antigens. Visual inspection of the structure model allows regions of protein that are both exposed and associated with the putative mechanism of action of the protein to be identified. Peptides corresponding to these regions of the protein can then be synthesised and employed as antigenic agents for the generation of monoclonal antibodies by routine methods. Elicited monoclonal antibodies may be tested using, for example, an appropriate ELISA assay to determine whether the monoclonal antibodies bind to RSV. Further, the capacity of elicited monoclonal antibodies to neutralise RSV may be assessed by conducting standard virus neutralisation tests in vivo using susceptible animals, or otherwise, in tissue cultures.

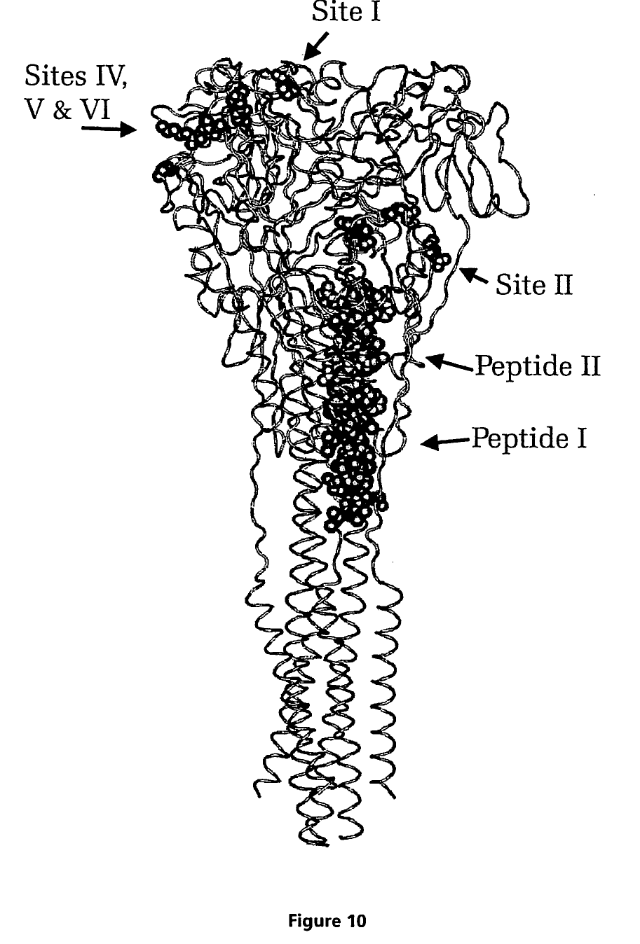

Antigenic sites on the RSV-F protein have been found to duster in immunogenic hotspots (Collins, P. L. et al., p. 1313-135I In B. N. Fields, D. M. Knipe, P. M. Howely (ed.) Fields Virology, 3rd Edition, Lippincott-Raven, Phil., 1996; Lopez, J. A. et al., J. Gen. Virol. 77:649-660, 1996), and the positions of some of these sites investigated by electron microscopy of RSV-F-antibody complexes (Calder, L. J. et al, Virology 271:122-131, 2000). Several of these were mapped on the RSV-F structure model (FIG. 10). The EM studies of antibody-bound RSV-F identified the approximate position for five different antigenic determinants on the protein; site I towards the top of the head, site II at the base of the neck and sites IV, V and VI overlapping on the side of the head (see FIG. 4 in Calder etal., supra). Antibody binding at site I is abrogated by the mutation of residue 389, which is found at the top of the head region, adjacent to the central channel. Site II is characterised by a duster of antibody escape mutations including residues 262, 268, 272 and 275 which form part of a sequence identified as one of the major determinants of RSV antigenticity in vivo. This section of the RSV-F protein forms a helix-turn-helix structure in the structure model that is fully exposed on the edge of one of the radial channels. The other three binding sites investigated by EM overlap and are identified by escape mutants at residues 429, 432, 433, 436 and 437. These residues form an exposed strand on the side of the head region of the RSV-F structure model (FIG. 10). The position of the antibody binding sites, both relative to each other and to the RSV-F structure, is identical in the RSV-F structure model and the experimental EM data.

A discrepancy is apparent between the RSV-F structure model and the predicted location of another immunogenic region comprising residues 205 to 237 (FIG. 10, Peptides I and II). These residues are almost totally concealed within the neck region of the RSV-F structure model, with only approximately 1000 Å² of a total potential surface of 6500 Å² exposed. Two overlapping peptides, ie Peptides I and II (FIG. 10), from this region react with neutralising sera from RSV infected animals and induce neutralising antibodies to RSV (Collins et al., supra). All of the surface exposed in the structure model originates in residues from the first peptide (residues 205-221, FIG. 10, dark grey) with residues in the second peptide (222-237, FIG. 10, light grey) completely buried. As these residues are in an area of high confidence in the structure model, this observation implies that antibodies specific to these regions interact with a different conformation of RSV-F, such as the hypothesised pre-fusogenic form of the protein or some intermediate that appears during the conversion from the pre-fusogenic to the fusogenic form.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for identifying a candidate anti-viral agent, said method comprising the steps of:
(i) generating a three-dimensional structure model of RSV-F protein or a portion thereof, wherein said structure model comprises the three-dimensional structure of a target site to which an anti-viral agent may interact and thereby inhibit the activity of said RSV-F protein; and
(ii) identifying a candidate anti-viral agent by designing or selecting a compound or chemical complex with a three-dimensional structure enabling interaction with said target site.

2. The method of claim 1, further comprising a step of assessing the deformation of energy of a candidate anti-viral agent when brought from a free state to a target site-interacting state.

3. The method of claim 2, wherein the candidate anti-viral agent has a deformation of energy of not greater than 10 kcal/mole.

4. The method of claim 1 or 2, wherein in addition to, or in the alternative to, the step of assessing the deformation of a candidate anti-viral agent, the method comprises a step assessing the enthalpy of the interaction of a candidate anti-viral agent with the target site.

5. The method of claim 4, wherein the candidate anti-viral agent has a neutral or favourable contribution to the enthalpy of the interaction.

6. A method for screening compounds and/or chemical complexes for a candidate anti-viral agent, said method comprising the steps of:
(i) generating a three-dimensional structure model of RSV-F protein or a portion thereof, wherein said structure model comprises the three-dimensional structure of a target site to which an anti-viral agent may interact and thereby inhibit the activity of said RSV-F protein; and
(ii) screening said compounds and/or chemical complexes to identify any compound(s) or chemical complex(es) having a three-dimensional structure which enables interaction with said target site.

7. The method of claim 6, wherein the screened compounds and/or chemical complexes belong to a library or database of suitable compounds and/or chemical complexes.

8. The method of any one of claims 1-7, wherein the candidate anti-viral agent shows inhibitory activity against RSV.

9. The method of any one of claims 1-7, wherein the candidate anti-viral agent shows inhibitory activity against viruses with fusion proteins showing at least 20% homology to RSV-F protein.

10. The method of claim 9, wherein the candidate anti-viral agent shows inhibitory activity against a virus belonging to the Paramyxoviridae family of viruses.

11. The method of any one of claims 1-10, wherein the three-dimensional structure model comprises, at least, the three-dimensional structure of a target site comprising all or part of each of the following amino acids of RSV-F protein; Tyr33, Cys37, Ser38, Ala39, Val40, Ser41, Lys42, Gly43, Leu48, Arg49, Thr50, Lys315, Leu316, His317, Thr318, Ser319, Pro320, Leu321, Cys322, Thr323, Ser330, Asn331, Ile332, Cys333, Leu334, Thr335, Arg336, Thr337, Asp338, Arg339, Phe352, Pro353, Gln354, Ala355, Glu356, Thr357, Cys358, Phe366, Cys367, Asp368, Thr369, Met370, Asn371, Ser372, Leu373, Lys394, Ile395, Met396, Thr397, Ser398, Lys399, Thr400, Asp401, Val402, Ser403, Ser404, Ser405, Val406, Ile407, Thr408, Ser409, Leu410, Gly411, Ala412, Ile413, Val414, Ser415, Lys419, Lys421 and Asp440 (as derived from the amino add sequence shown as SEQ ID NO:1), or the corresponding amino acids of F protein from variant RSV strains.

12. The method of any one of claims 1-11, wherein the three-dimensional structure model is generated using at least the atomic coordinate data of Table 1.

13. The method of any one of claims 1-11, wherein the three-dimensional structure model is generated from the atomic coordinate data of Table 2.

14. The method of any one of claims 1-13, wherein the method is performed in silico.

15. A method for modifying a candidate anti-viral agent to provide an agent with improved anti-viral activity, said method comprising the steps of:
i) generating a three-dimensional structure model of RSV-F protein or a portion thereof, wherein said structure model comprises the three-dimensional structure of a target site to which the candidate anti-viral agent interacts and thereby inhibits the activity of said RSV-F protein; and
(ii) modifying the candidate anti-viral agent to provide an agent with a three-dimensional structure more favourable to interaction with said target site or an agent which may otherwise interact more strongly with the target site than the candidate anti-viral agent.

16. The method of claim 15, wherein the step of modifying the candidate anti-viral agent is achieved by substituting one or more functional groups.

17. The method of claim 15 or 16, wherein the modified anti-viral agent shows inhibitory activity against RSV.

18. The method of claim 15 or 16, wherein the modified anti-viral agent shows inhibitory activity against viruses with fusion proteins showing at least 20% homology to RSV-F protein.

19. The method of claim 18, wherein the modified anti-viral agent shows inhibitory activity against a virus belonging to the Paramyxoviridae family of viruses.

20. The method of any one of claims 15-19, wherein the three-dimensional structure model comprises, at least, the three-dimensional structure of a target site comprising all or part of each of the following amino acids of RSV-F protein; Tyr33, Cys37, Ser38, Ala39, Val40, Ser41, Lys42, Gly43, Leu48, Arg49, Thr50, Lys315, Leu316, His317, Thr318, Ser319, Pro320, Leu321, Cys322, Thr323, Ser330, Asn331, Ile332, Cys333, Leu334, Thr335, Arg336, Thr337, Asp338, Arg339, Phe352, Pro353, Gln354, Ala355, Glu356, Thr357, Cys358, Phe366, Cys367, Asp 368, Thr369, Met370, Asn371, Ser372, Leu373, Lys394, Ile395, Met396, Thr397, Ser398, Lys399, Thr400, Asp401, Val402, Ser403, Ser404, Ser405, Val406, Ile407, Thr408, Ser409, Leu410, Gly411, Ala412, Ile413, Val414, Ser415, Lys419, Lys421 and Asp440 (as derived from the amino acid sequence shown as SEQ ID NO:1), or the corresponding amino acids of F protein from variant RSV strains.

21. The method of any one of claims 15-20, wherein the three-dimensional structure model is generated using at least the atomic coordinate data of Table 1.

22. The method of any one of claims 15-20, wherein the three-dimensional structure model is generated from the atomic coordinate data of Table 2.

23. The method of any one of claims 15-22, wherein the method is performed in silico.

24. A computer for producing a three-dimensional structure model of RSV-F protein or a portion thereof, said structure model comprising the three-dimensional structure of a target site to which an anti-viral agent may interact and thereby inhibit the activity of said RSV-F protein, wherein said computer comprises:
(i) a machine-readable data storage medium comprising the atomic coordinate data of the following amino acids of RSV-F protein; Tyr33, Cys37, Ser38, Ala39, Val40, Ser41, Lys42, Gly43, Leu48, Arg4⁹, Thr50, Lys315, Leu316, His317, Thr318, Ser319, Pro320, Leu321, Cys322, Thr323, Ser330, Asn331, Ile332, Cys333, Leu334, Thr335, Arg336, Thr337, Asp338, Arg339, Phe352, Pro353, Gln354, Ala355, Glu356, Thr357, Cys358, Phe366, Cys367, Asp368, Thr369, Met370, Asn371, Ser372, Leu373, Lys394, Ile395, Met396, Thr397, Ser398, Lys399, Thr400, Asp401, Val402, Ser403, Ser404, Ser405, Val406, Ile407, Thr408, Ser409, Leu410, Gly411, Ala412, Ile413, Val414, Ser415, Lys419, Lys421 and Asp440 (as derived from the amino acid sequence shown as SEQ ID NO:1), or the corresponding amino acids of F protein from variant RSV strains;
(ii) a working memory for storing instructions for processing said atomic coordinate data contained on the machine-readable data storage medium;
(iii) a central processing unit coupled to said working memory and to said machine-readable data storage medium for processing said atomic coordinate data to generate said three-dimensional structure model; and
(iv) a display coupled to said central processing unit for displaying a representation of said three-dimensional structure model.

25. The computer of claim 24, further comprising:
(v) means for receiving and storing atomic coordinate data for a range of chemical components and substituents, wherein the central processing unit is capable of interacting with said receiving and storing means and selects from said range of chemical components and substituents suitable chemical components and substituents to assemble a compound or chemical complex which, based upon a three-dimensional structure generated by said central processing unit, a representation of which may be provided on said display simultaneously with the representation of said three-dimensional structural model of RSV-F protein or a portion thereof, is capable of interaction with said target site; and/or
(vi) means for receiving and storing atomic coordinate data for a range of compounds and/or chemical complexes, wherein the central processing unit is capable of interacting with said receiving and storing means to generate a three-dimensional structure for a compound or chemical complex selected from the range of compounds and/or chemical complexes, provide a representation of said three-dimensional structure on said display simultaneously with the representation of said three-dimensional structural model of RSV-F protein or a portion thereof, and thereby enable an assessment of whether said selected compound or chemical complex is capable of interaction with said target site.

26. The computer of claim 24 or 25, wherein the machine-readable data storage medium comprises at least the atomic coordinate data of Table 1.

27. The computer of claim 24 or 25, wherein the machine-readable data storage medium comprises the atomic coordinate data of Table 2.

28. A machine-readable data storage medium comprising the atomic coordinate data of the following amino acids of RSV-F protein; Tyr33, Cys37, Ser38, Ala39, Val40, Ser41, Lys42, Gly43, Leu48, Arg49, Thr50, Lys315, Leu316, His317, Thr318, Ser319, Pro320, Leu321, Cys322, Thr323, Ser330, Asn331, Ile332, Cys333, Leu334, Thr335, Arg336, Thr337, Asp338, Arg339, Phe352, Pro353, Gln354, Ala355, Glu356, Thr357, Cys358, Phe366, Cys367, Asp368, Thr369, Met370, Asn371, Ser372, Leu373, Lys394, Ile395, Met396, Thr397, Ser398, Lys399, Thr400, Asp401, Val402, Ser403, Ser404, Ser405, Val406, Ile407, Thr408, Ser409, Leu410, Gly411, Ala412, Ile413, Val414, Ser415, Lys419, Lys421 and Asp440 (as derived from the amino acid sequence shown as SEQ ID NO:1), or the corresponding amino acids of F protein from variant RSV strains.

29. The machine-readable data storage medium of claim 28, comprising at least the atomic coordinate data of Table 1.

30. The machine-readable data storage medium of claim 28, comprising the atomic coordinate data of Table 2.

31. A method for producing an anti-viral agent, said method comprising the steps of:
(i) generating a three-dimensional structure model of RSV-F protein or a portion thereof, wherein said structure model comprises the three-dimensional structure of a target site to which an anti-viral agent may interact and thereby inhibit the activity of said RSV-F protein;
(ii) identifying an anti-viral agent by designing or selecting a compound or chemical complex with a three-dimensional structure enabling interaction with said target site; and
(iii) synthesising or isolating said identified anti-viral agent.

32. A method for producing an anti-viral agent, said method comprising the steps of:
(i) generating a three-dimensional structure model of RSV-F protein or a portion thereof, wherein said structure model comprises the three-dimensional structure of a target site to which an anti-viral agent may interact and thereby inhibit the activity of said RSV-F protein;
(ii) screening compounds and/or chemical complexes to identify any compound(s) or chemical complex(es) having a three-dimensional structure which enables interaction with said target site; and
(iii) synthesising or isolating said identified compound(s) or chemical complex(es) to provide said anti-viral agent.

33. The method of claim 31 or 32, wherein the anti-viral agent shows inhibitory activity against RSV.

34. The method of claim 32 or 33, wherein the anti-viral agent shows inhibitory activity against viruses with fusion proteins showing at least 20% homology to RSV-F protein.

35. The method of claim 34, wherein the anti-viral agent shows inhibitory activity against a virus belonging to the Paramyxoviridae family of viruses.

36. The method of any one of claims 32-35, wherein the three-dimensional structure model generated in step (i) comprises, at least, the three-dimensional structure of a target site comprising all or part of each of the following amino acids of RSV-F protein; Tyr33, Cys37, Ser38, Ala39, Val40, Ser41, Lys42, Gly43, Leu48, Arg49, Thr50, Lys315, Leu316, His317, Thr318, Ser319, Pro320, Leu321, Cys322, Thr323, Ser330, Asn331, Ile332, Cys333, Leu334, Thr335, Arg336, Thr337, Asp338, Arg339, Phe352, Pro353, Gln354, Ala355, Glu356, Thr357, Cys358, Phe366, Cys367, Asp368, Thr369, Met370, Asn371, Ser372, Leu373, Lys394, Ile395, Met396, Thr397, Ser398, Lys399, Thr400, Asp401, Val402, Ser403, Ser404, Ser405, Val406, Ile407, Thr408, Ser409, Leu410, Gly411, Ala412, Ile4113, Val414, Ser415, Lys419, Lys421 and Asp440 (as derived from the amino acid sequence shown as SEQ ID NO:1), or the corresponding amino acids of F protein from variant RSV strains.

37. The method of any one of claims 32-36, wherein the three-dimensional structure model generated in step (i) is generated using at least the atomic coordinate data of Table 1.

38. The method of any one of claims 32-36, wherein the three-dimensional structure model is generated from the atomic coordinate data of Table 2.

39. A candidate anti-viral agent identified in accordance with the method of any one of claims 1-14.

40. An anti-viral agent identified in accordance with the method of any one of claims 15-23 and 31-38.

41. A medicament for treating a viral infection in a subject, comprising the agent of claim 39 or 40.

42. The use of the agent of claim 39 or 40 in the preparation of a medicament for treating a viral infection in a subject.

43. A method of treating a viral infection in a subject, said method comprising administering the medicament of claim 41 to said subject.

44. A method of identifying a region within the RSV-F protein comprising an antigenic determinant which may be used to elicit antibodies capable of binding RSV, said method comprising the steps of:
(i) generating a three-dimensional structure model of RSV-F protein; and
(ii) identifying from said structure model a surface-located region of the RSV-F protein which may include an antigenic determinant.

45. The method of claim 44, wherein the three-dimensional structure model is generated from the atomic coordinate data of Table 2.

46. The method of claim 44 or 45, wherein the identified surface-located region of RSV-F protein comprises 5 to 100 amino adds.

47. The method of any one of claims 44-46, further comprising the steps of:
(iii) producing and isolating antibodies elicited by said surface-located region of RSV-F protein; and
iv) assessing said antibodies for RSV binding capacity.

48. The method of any one of claims 44116, further comprising the steps of:
(iii) producing and isolating antibodies elicited by said surface-located region of RSV-F protein; and
(iv) assessing said antibodies for RSV neutralising capacity.

49. An antibody or fragment thereof which binds RSV, wherein said antibody has been elicited by a surface-located region of RSV-F protein identified by the method of claim 47.

50. An antibody or fragment thereof which binds RSV, wherein said antibody has been elicited by a surface-located region of RSV-F protein identified by the method of claim 48.

51. A method of detecting RSV in a sample, the method comprising exposing said sample to the antibody or fragment thereof of claim 49, and subsequently detecting binding of said antibody or fragment thereof to RSV or RSV-F protein.

52. A medicament for treating or preventing viral infection in a subject, comprising the antibody or fragment thereof of claim 50 optionally in combination with a suitable carrier.

53. The use of the antibody or fragment thereof of claim 50 in the preparation of a medicament for treating or preventing viral infection in a subject.

53. A method of treating or preventing a viral infection in a subject, said method comprising administering the medicament of claim 52 to said subject.

54. A vaccine agent comprising a peptide or polypeptide which comprises an amino acid sequence which corresponds to a surface-located region of RSV-F protein identified by the method of claim 48, optionally in combination with a suitable adjuvant or carrier.

55. The vaccine agent of claim 54, wherein the surface-located region of RSV-F protein comprises 5 to 100 amino acids.

56. A recombinant viral vector comprising a nucleotide sequence encoding an amino acid sequence which corresponds to a surface-located region of RSV-F protein identified by the method of claim 48, optionally in combination with a suitable adjuvant or carrier.

57. The viral vector of claim 56, wherein the surface-located region of RSV-F protein comprises 5 to 100 amino acids.

58. A method of treating or preventing a viral infection in a subject, said method comprising administering the vaccine agent of claim 54 or 55 or the viral vector of claim 56 or 57.


SEQUENCE LISTING (3 sequences)

SEQ ID NO: 1 - protein, 574 aa, respiratory syncytial virus
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 2 - protein, 90 aa, respiratory syncytial virus
LEGEVNKIKSALLSTNKAVVSLSNGVSVLTSKVLDLKNYIDKQLLPIVNKPLVFPSDEFDASISQVNEKINQSLAFIRKSDELLHNVNAG

SEQ ID NO: 3 - protein, 553 aa, Newcastle disease virus
MGSRSSTRIPVPLMLTVRVMLALSCVCPTSALDGRPLAAAGIVVTGDKAVNIYTSSQTGSIIIKLLPNMPKDKEACAKAPLEAYNRTLTTLLTPLGDSIRRIQESVTTSGGGKQGRLIGAIIGGVALGVATAAQITAASALIQANQNAANILRLKESIAATNEAVHEVTNGLSQLAVAVGKMQQFVNDQFNKTAQELDCIKITQQVGVELNLYLTELTTVFGPQITSPALTQLTIQALYNLAGGNMDYLLTKLGVGNNQLSSLISSGLITGNPILYDSQTQLLGIQVTLPSVGNLNNMRATYLETLSVSTTKGFASALVPKVVTQVGSVIEELDTSYCIETDLDLYCTRIVTFPMSPGIYSCLSGNTSACMYSKTEGALTTPYMTLKGSVIANCKMTTCRCADPPGIISQNYGEAVSLIDRQSCNILSLDGITLRLSGEFDATYQKNISIQDSQVIVTGNLDISTELGNVNNSISNALDKLEESNSKLDKVNVKLTSTSALITYIVLTVISLVCGILSLVLACYLMYKQKAQQKTLLWLGNNTLDQMRATTKM